(19) United States
(12) Patent Application Publication
(10) Pub. No.: US 2026/0364102 A1
(43) Pub. Date: Nov. 19, 2026

(54) IN VIVO NICKASE-BASED EDITING OF THE LPA GENE FOR TREATMENT OF CARDIOVASCULAR DISEASE

(71) Applicant: Verve Therapeutics, Inc., Boston, MA (US)

(72) Inventors: Peter Thomas Hallock, Boston, MA (US); Liam Cornell, Sommerville, MA (US); Sara Cristina de Almeida Pinto Garcia, Brookline, MA (US); Srinidhi Sridhar, Quincy, MA (US); Caroline Summers, Concord, MA (US); Kui Wang, Wellesley, MA (US); Christopher J. Cheng, North Reading, MA (US)

(21) Appl. No.: 19/166,792

(22) PCT Filed: Mar. 20, 2024
(86) PCT No.: PCT/US2024/020769

§ 371 Date: Sep. 18, 2025

Related U.S. Application Data
(60) Provisional application No. 63/453,207, filed on Mar. 20, 2023.
(60) Provisional application No. 63/554,838, filed on Feb. 16, 2024.

(51) Int. Cl. A61K 31/7105; A61K 9/51; A61K 38/46; C12N 9/22; C12N 15/11; C12N 15/88

(52) U.S. Cl. CPC A61K 31/7105; A61K 9/5123; A61K 9/5146; A61K 38/465; C12N 9/226; C12N 15/11; C12N 15/88; C12N 2310/20

(57) ABSTRACT

Provided herein are gene editing systems and compositions directed to effectuate in vivo edits in the LPA gene. Treatment or prevention of cardiovascular disease through disruption of the production of apo(a) through genetic editing and the reduction of the blood lipoprotein(a) [Lp(a)] concentration is disclosed herein. Disclosed are nickase-based gene editing systems designed to effectuate the installation of insertions and/or deletions (indel variants) and/or non-synonymous variants in the coding sequence of LPA. The nickase-based gene editing systems generally comprise one or more mRNAs that encode one or more nickases and a plurality of guide oligonucleotides (e.g., gRNAs) and may be delivered in vivo to a mammalian subject in need thereof via a suitable delivery system, such as lipid nanoparticles (LNPs) (with or without GalNAc targeting moieties) intravenously, or otherwise, administered to a patient as potentially a once-and-done therapeutic. The manufacturing, use, and formulation of the gene editing systems and compositions are also disclosed.

### RELATED APPLICATIONS

This application claims the benefit of U.S. Provisional Patent Application No. 63/453,207, filed on Mar. 20, 2023, and U.S. Provisional Patent Application No. 63/554,838, filed on Feb. 16, 2024, which applications are hereby incorporated herein by reference in their respective entireties.

### BACKGROUND

Lipoprotein(a) [Lp(a)] is a low-density lipoprotein (LDL) particle comprising apolipoprotein(a) [apo(a)] covalently linked to apolipoprotein B-100 (apoB-100), which is the primary protein component of LDL particles. The apo(a) protein is encoded by the LPA gene, which is specifically expressed in hepatocytes in the liver of humans and certain non-human primates and secreted into the bloodstream where it is a constituent component of Lp(a). LDL cholesterol (the quantified aggregate cholesterol content of LDL particles circulating in the bloodstream) is a well-established casual risk factor for atherosclerotic cardiovascular disease (ASCVD), which remains a leading cause of death worldwide notwithstanding the existence of approved chronic care drugs, including statins, ezetimibe, and proprotein convertase subtilisin/kexin type 9 (PCSK9) inhibitors. Blood Lp(a) concentration has been established to be a specific causal risk factor for ASCVD, and there is no approved therapy that specifically targets LPA, apo(a), or Lp(a). Furthermore, unlike LDL cholesterol, Lp(a) has been established to be a causal risk factor for calcific aortic valve disease, a distinct type of cardiovascular disease characterized by the stiffening or thickening of the aortic valves.

### SUMMARY

The present application discloses novel gene editing technology, including gene editor mRNA, guide oligonucleotides (e.g., guide RNA or “gRNA”), gene editing and delivery systems, and components, formulations, and pharmaceutical compositions thereof, which, alone and/or in combination, constitute separate aspects of the inventive subject matter disclosed herein. The disclosed gene editing technology is capable of in vitro and in vivo editing of the LPA gene, including specifically LPA genes found in human hepatocytes of the liver. Various embodiments are disclosed that are directed to inactivating the LPA gene via the introduction of loss-of-function LPA variants that disrupt the production of apo(a) and/or Lp(a) protein thereby reducing the blood apo(a) and Lp(a) concentration to the substantial benefit of patients, such as those with existing cardiovascular disease or at risk of developing cardiovascular disease.

Accordingly, the gene editing and delivery systems, their respective components and elements, and the manufacture and/or use of those systems and components, described herein, alone and/or in any combination constitute individual aspects of the inventive subject matter disclosed herein. Thus, the use of the compositions and methods disclosed herein to disrupt the production of apo(a) and/or Lp(a) protein and/or prevent and/or treat cardiovascular disease constitutes separate aspects of the inventive subject matter disclosed herein. The location, nature and/or degree of the disruption of the LPA gene and/or commensurate disruption of the production of apo(a) and Lp(a) further constitute aspects of the inventive subject matter disclosed herein.

In some embodiments, the invention provides pharmaceutical composition for in vivo editing of an LPA gene in a mammalian subject comprising an engineered, non-naturally occurring gene editing system and a delivery system. In embodiments, the gene editing system includes one or more polynucleotides (e.g., mRNAs) encoding one or more CRISPR Cas nickases, a first guide oligonucleotide (e.g., gRNA), and a second guide oligonucleotide (e.g., gRNA). The first guide oligonucleotide comprises a first spacer sequence and a first scaffold region. The first spacer sequence is complementary to a first strand of the LPA gene at a first target sequence. The first scaffold region serves as a binding scaffold for at least one of the one or more Cas nickases. The second guide oligonucleotide comprises a second spacer sequence and a second scaffold region. The second spacer sequence is complementary to a second strand of the LPA gene at a second target sequence. The second scaffold region serves as a binding scaffold for at least one of the one or more Cas nickases. The delivery system is engineered to deliver the one or more polynucleotides encoding one or more CRISPR Cas nickases, the first guide oligonucleotide, and/or the second guide oligonucleotide individually and/or collectively to the liver. The first guide oligonucleotide and the at least one of the one or more Cas nickases are engineered to cause the at least one of the one or more Cas nickases to nick one of the first or second strands of the LPA gene at a first location of human chromosome 6 from position 160,664,275 to 160,531,482. The second guide oligonucleotide and the at least one of the one or more Cas nickases are engineered to cause the at least one of the Cas nickases to nick the other of the first or second strands of the LPA gene at a second location of chromosome 6 from position 160,664,275 to 160,531,482.

In some embodiments, expression of the edited LPA gene results in lowered production or lack of production of apo(a) protein in cells in which the LPA gene is edited. In some embodiments, expression of the edited LPA gene results in lowered production or lack of production of apo(a) protein in hepatocytes. In some embodiments, the hepatocytes are primary hepatocytes.

In some embodiments, expression of the edited LPA gene results in lowered amounts of LPA RNA, such as mRNA or pre-mRNA, in a cell in which the LPA gene is edited. Without intending to be bound by theory, it is believed that transcription of the edited LPA gene may, in some embodiments, result in nonsense-mediated decay of the LPA RNA.

In some embodiments, expression of the edited LPA gene results in a lowering or a lack of production of apo(a) protein or the production of non-functional apo(a) protein, resulting in a reduced blood Lp(a) concentration. In some embodiments, the blood Lp(a) concentration is reduced to an extent to treat ASCVD and/or calcific aortic valve disease. In embodiments, Lp(a) concentration in plasma is reduced. In embodiments, blood concentration in serum is reduced.

Embodiments of the invention are directed to pharmaceutical compositions comprising one or more components (e.g., mRNA, gRNAs) of the LPA gene editing system, including pharmaceutical compositions thereof that allow for in vivo delivery. In an illustrative embodiment, the delivery system for the LPA gene editing system comprises lipid nanoparticles (LNPs) comprising: (a) one or more ionizable lipids, (b) cholesterol, (c) one or more PEG-lipids, (d) a phospholipid; and optionally including a targeting moiety, such as a GalNAc lipid.

In embodiments, the Cas nickase is a Cas9 nickase. In some embodiments, the Cas9 nickase, when in operative interaction with the first or second guide oligonucleotide (e.g., gRNA), is engineered to nick the opposite strand of the LPA gene to which the operative guide is hybridized. In some embodiments, the Cas9 nickase, when in operative interaction with the first or second guide oligonucleotide, is engineered to nick the same strand of the LPA gene to which the operative guide oligonucleotide (e.g., gRNA) is hybridized.

In illustrative embodiments, the Cas nickase comprises a Streptococcus pyogenes Cas9 nickase bearing a D10A mutation encoded within the polynucleotide (e.g., mRNA).

In some embodiments, the first guide oligonucleotide (e.g., gRNA) has a first spacer sequence that is substantially identical to a first protospacer sequence adjacent to a first protospacer-adjacent motif (PAM) sequence on one strand of the LPA gene, and the second guide oligonucleotide (e.g., gRNA) has a second spacer sequence that is substantially identical to a second protospacer sequence adjacent to a second protospacer-adjacent motif (PAM) sequence on the other strand of the LPA gene, and wherein the Cas9 nickase bearing a D10A mutation, in operation with the first and second guide oligonucleotides, makes two nicks on opposing strands (e.g., antisense and sense strands) of the LPA gene between the first and second protospacer-adjacent motifs (PAMs) in a “PAM-out” configuration (see, e.g., FIG. 1A).

In some embodiments, the first guide oligonucleotide (e.g., gRNA) has a first spacer sequence that is substantially identical to a first protospacer sequence adjacent to a first protospacer-adjacent motif (PAM) sequence on one strand of the LPA gene, and the second guide oligonucleotide (e.g., gRNA) has a second spacer sequence that is substantially identical to a second protospacer sequence adjacent to a second protospacer-adjacent motif (PAM) sequence on the other strand of the LPA gene, and wherein the Cas9 nickase bearing a D10A mutation, in operation with the first and second guide oligonucleotides, makes two nicks on opposing strands (e.g., antisense and sense strands) of the LPA gene outside the first and second protospacer-adjacent motifs (PAMs) in a “PAM-in” configuration (see, e.g., FIG. 1C).

In some embodiments, the Cas nickase comprises Streptococcus pyogenes Cas9 nickase bearing a H840A mutation encoded within the polynucleotide (e.g., mRNA).

In some embodiments, the first guide oligonucleotide (e.g., gRNA) has a first spacer sequence that is substantially identical to a first protospacer sequence adjacent to a first protospacer-adjacent motif (PAM) sequence on one strand of the LPA gene, and the second guide oligonucleotide (e.g., gRNA) has a second spacer sequence that is substantially identical to a second protospacer sequence adjacent to a second protospacer-adjacent motif (PAM) sequence on the other strand of the LPA gene, and wherein the Cas9 nickase bearing a H840A mutation, in operation with the first and second guide oligonucleotides, makes two nicks on the first and second protospacer sequences on opposing strands (e.g., antisense and sense strands) of the LPA gene between the first and second protospacer-adjacent motifs (PAMs) in a “PAM-out” configuration (see, e.g., FIG. 1B).

In some embodiments, the first guide oligonucleotide (e.g., gRNA) has a first spacer sequence that is substantially identical to a first protospacer sequence adjacent to a first protospacer-adjacent motif (PAM) sequence on one strand of the LPA gene, and the second guide oligonucleotide (e.g., gRNA) has a second spacer sequence that is substantially identical to a second protospacer sequence adjacent to a second protospacer-adjacent motif (PAM) sequence on the other strand of the LPA gene, and wherein the Cas9 nickase bearing a H840A mutation, in operation with the first and second guide oligonucleotides, makes two nicks on the first and second protospacer sequences on opposing strands (e.g., sense and antisense strands) of the LPA gene outside the first and second protospacer-adjacent motifs (PAMs) in a “PAM-in” configuration (see, e.g., FIG. 1D).

In some embodiments, the Cas9 nickase in operation with the first and second guide nucleotides nicks the DNA to generate nicks on opposing strands resulting in 5′ overhangs (e.g., as shown in FIGS. 1A and 1D). In some embodiments, the Cas9 nickase in operation with the first and second guide nucleotides nicks the DNA to generate nicks on opposing strands resulting in 3′ overhangs (e.g., as shown in FIGS. 1B and 1C). The overhangs may have any length suitable to allow non-homologous end-joining. The length of the overhangs is defined by the distance between the nicks in DNA strands. It will be understood that the length of the overhangs suitable to allow non-homologous end-joining may vary depending on a number of factors. In some embodiments, the overhangs have a length from 1 to 200 nucleotides, such as 10 to 150 nucleotides, 15 to 100 nucleotides, or 20 to 50 nucleotides. In some embodiments, the overhangs have a length of 10 nucleotides or greater, such as 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or greater. In some embodiments, the overhangs have a length of 200 nucleotides or less, such as 190, 180, 170, 160, 150, 140, 130, 120, 110, 100, 90, 80, 70, 60, or 50 nucleotides or less. In some embodiments, the overhangs have a length of 20 to 50 nucleotides, such as 23 to 45, 30 to 40, 31 to 40, 32 to 40, 33 to 40, 31 to 39, 32 to 39, 33 to 39, or 34 to 38 nucleotides.

In some embodiments, the sequences of the first and second scaffold regions of the first and second guide oligonucleotides are the same. In some embodiments, the sequences of the first and second scaffold regions are different.

In embodiments, the in vivo LPA gene editing system comprises one polynucleotide (e.g., mRNA) encoding one CRISPR Cas9 nickase. In some embodiments, the in vivo LPA gene editing system further comprises one or more polynucleotides (e.g., mRNA), each encoding a CRISPR Cas nickase.

In embodiments, the in vivo LPA gene editing system comprises a Cas nickase that is engineered to be directed to protospacer sequences within or in proximity to exon 20 of the LPA gene, which spans from chromosome 6, position 160,599,659, to chromosome 6, position 160,599,500, by the first and second guide oligonucleotides (e.g., gRNAs). In some such embodiments, the gene editing system is configured to effect indel variants and/or non-synonymous variants in the LPA gene.

In embodiments, the in vivo LPA gene editing system comprises a Cas nickase that is engineered to be directed to protospacer sequences within or in proximity to exon 23 of the LPA gene, which spans from chromosome 6, position 160,591,101, to chromosome 6, position 160,590,944, by the first and second guide oligonucleotides (e.g., gRNAs). In some such embodiments, the gene editing system is configured to effect indel variants and/or non-synonymous variants in the LPA gene.

In embodiments, the in vivo LPA gene editing system comprises a Cas nickase that is engineered to be directed to protospacer sequences within or in proximity to exon 25 of the LPA gene, which spans from chromosome 6, position 160,586,630, to chromosome 6, position 160,586,449, by the first and second guide oligonucleotides (e.g., gRNAs). In some such embodiments, the gene editing system is configured to effect indel variants and/or non-synonymous variants in the LPA gene.

In embodiments, the in vivo LPA gene editing system comprises a Cas nickase that is engineered to be directed to protospacer sequences within or in proximity to exon 19 of the LPA gene, which spans from chromosome 6, position 160,601,098, to chromosome 6, position 160,600,917, by the first and second guide oligonucleotides (e.g., gRNAs). In some such embodiments, the gene editing system is configured to effect indel variants and/or non-synonymous variants in the LPA gene.

In embodiments, the in vivo LPA gene editing system comprises a Cas nickase that is engineered to be directed to protospacer sequences within or in proximity to exon 31 of the LPA gene, which spans from chromosome 6, position 160,548,659, to chromosome 6, position 160,548,478, by the first and second guide oligonucleotides (e.g., gRNAs). In some such embodiments, the gene editing system is configured to effect indel variants and/or non-synonymous variants in the LPA gene.

In embodiments, a plurality of different guide oligonucleotides (e.g., gRNAs) are used to effectuate the desired editing of the LPA gene. In some embodiments, the different guide oligonucleotides (e.g., gRNAs) are delivered in vivo to the target human liver cells/tissues simultaneously and operate to effectuate the editing of the LPA gene of human liver cells. In some embodiments, the different guide oligonucleotides (e.g., gRNAs) are delivered in vitro to the target human liver cells/tissues (e.g., HuH-7 cells) simultaneously and operate to effectuate the editing of the LPA gene of human liver cells. In some embodiments, one or more or all of the guide oligonucleotides (e.g., gRNAs) are delivered to the target liver tissue and liver cells via lipid nanoparticles (LNPs), which may comprise an ionizable lipid, cholesterol, PEG-lipid, and a phospholipid and may also include a targeting moiety, such as a GalNAc lipid. In some embodiments, the phospholipid comprises distearoylphosphatidylcholine (DSPC). The same or different LNPs may also serve to deliver mRNA encoding the gene editor, namely a Cas nickase (e.g., a Cas9 nickase). In embodiments, the mRNA and/or guide oligonucleotides (e.g., gRNAs) are selected from those specified herein. In embodiments, the Cas nickase (gene editor) and guide oligonucleotides (e.g., gRNAs) may be selected from gene editing systems and configurations illustrated in FIGS. 1A-ID.

In some embodiments, the in vivo gene editing system comprises mRNA that encodes a Cas nickase (gene editor) and comprises one or more guide oligonucleotides (e.g., gRNAs). A first guide oligonucleotide may comprise a first spacer sequence and a first scaffold region. A second guide oligonucleotide may comprise a second spacer sequence and a scaffold region that is different than or the same as the first scaffold region. The first spacer sequence may correspond with a first protospacer sequence and be designed to be complementary or otherwise hybridize to the strand complementary to the first protospacer to facilitate a nick by the nickase (e.g., Cas nickase) on one or the other DNA strand. The second spacer sequence may correspond to a second protospacer sequence that is located on the opposite strand to the first protospacer sequence and is in operational proximity to the first protospacer sequence, wherein the second spacer sequence is complementary or otherwise designed to hybridize to the strand complementary to the second protospacer to facilitate a nick by the nickase on the strand that does not receive a nick via the action of the nickase in connection with the first guide oligonucleotide.

In embodiments, the present invention provides isolated oligonucleotides (e.g., gRNAs) or nucleic acids encoding the same. The isolated oligonucleotides (e.g., gRNAs) each comprise (i) a spacer sequence comprising about 15 to about 26 nucleotides, such as about 17 to about 23 nucleotides, that is identical to or substantially identical to a targeted protospacer sequence adjacent to a protospacer-adjacent motif (PAM) sequence within an LPA gene; and (ii) a scaffold region. The isolated oligonucleotides serve as guide nucleic acids to direct the gene editor to effect the edit in the LPA gene. In embodiments, the edit or edits are configured to modify the LPA gene to effect indel variants or non-synonymous variants.

In some embodiments, the first protospacer is on the sense strand and the second protospacer is on the antisense strand of the LPA gene. In some embodiments, the first protospacer is on the antisense strand and the second protospacer is on the sense strand of the LPA gene.

In some embodiments, the mRNA that encodes a Cas nickase (gene editor) comprises: (a) a 5′ untranslated region (UTR); (b) a 3′ UTR region; (c) a poly(A) tail proximate to the 3′ UTR as compared to the 5′ UTR, said poly(A) tail comprising a chain of 80-150 nucleotides that comprise adenine nucleotides; and (d) a gene editor coding region encoding an impaired CRISPR Cas endonuclease domain, said gene editor coding region extending between the 5′ UTR and the 3′ UTR. In some embodiments, the gene editor coding region also encodes a polymerase domain.

In embodiments, the mRNA has 90% or greater sequence identity to any of the mRNA sequences listed in Table 1. For example, the mRNA may have 91% or greater, 92% or greater, 93% or greater, 94% or greater, 95% or greater, 96% or greater, 97% or greater, 98% or greater, or 99% or greater sequence identity to any of the mRNA sequences listed in Table 1.

In embodiments, the Cas nickase is selected from: a Streptococcus pyogenes Cas9 variant, a Staphylococcus aureus Cas9 variant, or a Cas12a/Cpf1 variant.

In embodiments, the present disclosure describes a method of effecting a modification in an LPA gene to result in loss of function. The method comprises delivering in vivo to a human liver cell, or administering to a mammalian subject, a pharmaceutical composition. The pharmaceutical composition comprises a polynucleotide (e.g., mRNA) encoding the Cas nickase, the first and second guide oligonucleotides (e.g., gRNAs), and the delivery system. In some embodiments, the delivery system is engineered to deliver the polynucleotide encoding the Cas nickase, the first guide oligonucleotide, and/or the second guide oligonucleotide individually or collectively to the liver. In some embodiments, the delivery system comprises an LNP. In other embodiments, the method comprises delivering in vitro to a human liver cell (e.g., HuH-7 cell), or administering to a mammalian subject, a pharmaceutical composition.

In embodiments, the LNP is formulated with an mRNA: guide oligonucleotide (e.g., gRNA) weight ratio of 1:1+/−5%, 10%, 15%, 20%, 30%, 40%, 50% 60%, 70%, 80%, 90% or 100% of the mRNA or guide oligonucleotide or any therapeutically effective ratio. A weight ratio of 1:1+/−100% includes weight ratios of 1:2 to 2:1. A 1:1 weight ratio of mRNA: guide oligonucleotide refers to the cumulative weight ratios of all mRNAs and all guide oligonucleotides. For example, if a single mRNA is included in the formulation and two guide oligonucleotides are included in the formulation, the weight ratio of the mRNA to the guide oligonucleotide will be the weight of the single mRNA relative to the combined weight of the two gRNAs in the formulation.

In embodiments, the LNP has an N/P ratio from about 4 to about 7, about 4, about 4.5, about 5, about 5.5, or about 6, about 6.5 or about 7 with each ratio +/−5-20%.

In embodiments, the buffer solution containing the LNP has a pH of about 7.5+/−1.5 and comprises tris and/or sucrose.

In embodiments, the LNP has a mean diameter of about 70 nm+/−20 nm, 70 nm+/−10 nm, 70 nm+/−5 nm; 60 nm+/−20 nm, 60 nm+/−10 nm, 60 nm+/−5 nm; 50 nm+/−20 nm, 50 nm+/−10 nm, 50 nm+/−5 nm; 45 nm+/−20 nm, 45 nm+/−10 nm, 45 nm+/−5 nm.

In embodiments, the present disclosure describes a method for in vivo editing of an LPA gene in a mammalian subject in need thereof comprising administering to the subject a pharmaceutical composition. The pharmaceutical composition comprises:
(i) a polynucleotide (e.g., mRNA) encoding a CRISPR Cas nickase,
(ii) a first guide oligonucleotide (e.g., gRNA) comprising a first spacer sequence and a scaffold region; and
(iii) a second guide oligonucleotide (e.g., gRNA) comprising a second spacer sequence and a scaffold region, and
(iv) a delivery system that is engineered to deliver the mRNA, the first gRNA, and/or the second gRNA individually and/or collectively to the liver, whereby in operation, the Cas nickase nicks each of the first and second strands of the LPA gene, in locations from chromosome 6, position 160,664,275, to chromosome 6, position 160,531,482.

In some embodiments, the mammalian subject is a human.

In embodiments, the subject has an elevated blood Lp(a) concentration. In embodiments, the subject has an inversely associated apo(a) concentration.

In embodiments, the subject has cardiovascular disease associated with an elevated blood Lp(a) concentration. In embodiments, the subject has cardiovascular disease associated with an inversely associated blood apo(a) concentration.

In embodiments, the in vivo editing of the LPA gene results in reduction of the blood Lp(a) concentration. In embodiments, the in vivo editing of the LPA gene results in an inversely associated blood apo(a) concentration.

In some embodiments, the invention provides a method for inactivating the LPA gene in vivo in a mammalian subject to treat and prevent cardiovascular disease comprising the step of: administering to the subject the pharmaceutical composition of the invention.

In some embodiments, the invention provides a method for reducing blood Lp(a) concentration or inversely associated apo(a) concentration in a mammalian subject to treat and prevent cardiovascular disease comprising the step of: administering to the subject a pharmaceutical composition of the invention.

In some embodiments, the invention provides a method for treating and/or preventing cardiovascular disease associated with the LPA gene in a mammalian subject comprising the step of: administering to the subject a pharmaceutical composition of the invention.

In some embodiments, the invention provides a gene editing system for editing the LPA gene. The gene editing system is produced by expressing in a cell one or more exogenous polynucleotides (e.g., mRNA) encoding one or more CRISPR Cas nickases and introducing first and second gRNAs into the cell. The first guide oligonucleotide (e.g., gRNA) comprises (i) a first spacer sequence that is complementary to a first strand of the LPA gene at a first target sequence and (ii) a first scaffold region that serves as a binding scaffold for at least one of the one or more Cas nickases. The second guide oligonucleotide (e.g., gRNA) comprises (i) a second spacer sequence that is complementary to a second strand of the LPA gene at a second target sequence and (ii) a second scaffold region that serves as a binding scaffold for at least one of the one or more Cas nickases. The first gRNA and the at least one of the one or more Cas nickases are engineered to cause the at least one of the one or more Cas nickases to nick one of the first or second strands of the LPA gene at a first location of chromosome 6 from position 160,664,275 to 160,531,482. The second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.

In some embodiments, the invention provides a gene editing system comprising (i) a means for expressing one or more CRISPR Cas nickases in a cell; and (ii) a means for directing the one or more Cas nickases to first and second locations in the LPA gene and to cause the one or more Cas nickases to introduce a nick in a first strand of the LPA gene and to introduce a nick in a second strand of the LPA gene.

In some embodiments, the invention provides a pharmaceutical composition for in vivo editing of an LPA gene, in a mammalian subject, comprising an engineered, non-naturally occurring gene editing system and a population of lipid nanoparticles that collectively encapsulate the gene editing system. The gene editing system comprises (i) a nickase or one or more polynucleotides (mRNAs) encoding a nickase; (ii) a first guide oligonucleotide (gRNA) comprising a first spacer sequence that includes a region that is complementary to a first strand of the LPA gene at a first target sequence and a first scaffold region that serves as a binding scaffold for the nickase; and (ii) a second guide oligonucleotide (gRNA) comprising a second spacer sequence that includes a region that is complementary to a second strand of the LPA gene at a second target sequence and a second scaffold region that serves as a binding scaffold for the nickase. The first and second strands are opposing strands. The first spacer has a sequence that is at least about 75%, at least about 80%, at least about 85%, at least about 90%, at least about 95% similar to or identical to, or is identical to the guide 1 protospacer listed in Table 2 or Table 5, and wherein the second spacer has a sequence that is at least about 75%, at least about 80%, at least about 85%, at least about 90%, at least about 95% similar to or identical to, or is identical to the guide 2 protospacer listed in Table 2 or Table 5.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE FIGURES

FIGS. 1A-1D are schematic drawings illustrating nickase-based editing systems and specifically dual nickase editing systems and configurations. Components of the gene editor and guide oligonucleotides (labeled as gRNAs) are identified and operationally described. In each of the configurations, the editing system effects single nicks on opposing DNA strands, which recruit DNA repair enzymes to facilitate non-homologous end-joining (NHEJ) repair to cause the edit (e.g., cause an indel variant or non-synonymous variant). FIG. 1A illustrates one system and configuration that employs a nickase Cas9 protein with mutation of the RuvC domain, exemplified by Streptococcus pyogenes (S. pyogenes) Cas9 with a D10A mutation, with two guide oligonucleotides. The first guide oligonucleotide has a spacer sequence that is identical to a first protospacer sequence on a bottom strand, and the second guide oligonucleotide has a spacer sequence that is identical to a second protospacer sequence on a top strand, with the 5′ end of the first spacer sequence and the 5′ end of the second spacer sequence being proximate to one another as compared to the 3′ ends of the spacers. The Cas9 nickase bearing a D10A mutation, in operation with the first and second guide oligonucleotides, makes two nicks on respective target sequences on the top and bottom strands of the gene (e.g., LPA) between the first and second protospacer-adjacent motifs (PAMs) in a “PAM-out” configuration, in which the protospacer-adjacent motifs (PAMs) for the two protospacer sequences are separated by the length of the region spanned by the protospacer sequences. That is, the two PAMs are distal relative to one another and flank the two protospacer sequences in a “PAM-out” configuration. FIG. 1B illustrates one system and configuration that employs a nickase Cas9 protein with mutation of the HNH domain, exemplified by S. pyogenes Cas9 with an H840A mutation, with two guide oligonucleotides. The first guide oligonucleotide has a spacer sequence that is identical to a first protospacer sequence on the bottom strand, and the second guide oligonucleotide has a spacer sequence that is identical to a second protospacer sequence on the top strand, with the 5′ end of the first spacer sequence and the 5′ end of the second spacer sequence being proximate to one another as compared to the 3′ ends of the spacers. The Cas9 nickase bearing a H840A mutation, in operation with the first and second guide oligonucleotides, makes a first nicks on the first protospacer sequence and a second nick on the second protospacer sequences on the respective bottom and top strands of the gene (e.g., LPA) between the first and second protospacer-adjacent motifs (PAMs) in a “PAM-out” configuration. Thus, as in FIG. 1A, the two PAMs depicted in FIG. 1B are distal relative to one another and flank the two protospacer sequences in a “PAM-out” configuration. FIG. 1C illustrates one system and configuration that employs a nickase Cas9 protein with mutation of the RuvC domain, exemplified by S. pyogenes Cas9 with a D10A mutation, with two guide oligonucleotides. The first guide oligonucleotide has a spacer sequence that is identical to a first protospacer sequence on the top strand, and the second guide oligonucleotide has a spacer sequence that is identical to a second protospacer sequence on the bottom strand, with the 3′ end of the first spacer sequence and the 3′ end of the second spacer sequence being proximate to one another as compared to the 5′ ends of the spacers. The Cas9 nickase bearing a D10A mutation, in operation with the first and second guide oligonucleotides, makes two nicks on respective target bottom and top strands of the LPA gene outside the first and second protospacer-adjacent motifs (PAMs) in a “PAM-in” configuration, in which the PAMs for the two protospacer sequences are proximate relative to one another and are flanked by the protospacer sequences. FIG. 1D illustrates one system and configuration that employs a nickase Cas9 protein with mutation of the HNH domain, exemplified by S. pyogenes Cas9 with an H840A mutation, with two guide oligonucleotides. The first guide oligonucleotide has a spacer sequence that is identical to a first protospacer sequence on the top strand, and the second guide oligonucleotide has a spacer sequence that is identical to a second protospacer sequence on the bottom strand, with the 3′ end of the first spacer sequence and the 3′ end of the second spacer sequence being proximate to one another as compared to the 5′ ends of the spacers. The Cas9 nickase bearing a H840A mutation, in operation with the first and second guide oligonucleotides, makes a first nick on the first protospacer sequence and a second nick on the second protospacer sequence on respective top and bottom strands of the LPA gene outside the first and second protospacer-adjacent motifs (PAMs) in a “PAM-in” configuration, in which the PAMs for the two protospacer sequences are proximate relative to one another and are flanked by the protospacer sequences. The arrows in the diagrams of FIGS. 1A-1D indicate the location of the nicks. It should be understood that the D10A nickase is representative of nickases that nick the target strand, while the H840A nickase is representative of nickases that nick the non-target strand (e.g., the strand containing the protospacer). Accordingly, it is contemplated that other nickases may be used in connection with the nickase-based editing systems described herein.

FIG. 2 is a plot of the percentages of LPA alleles edited in primary human hepatocytes (editing %, also referred to as editing efficiency) versus selected guide RNA pairs (first and second gRNAs) at relatively higher and lower doses, summarizing the LPA editing efficiencies in primary human hepatocytes using a gene editing system that comprises a Cas9 nickase with the specified pairs of first and second guide RNAs. Specific guide RNA pairs are ranked by editing efficiency at the higher 2500 ng/ml total RNA dose. The lower total RNA dose is 312.5 ng/ml. The Cas9 nickase is encoded within an mRNA (MS029) that was transfected into the primary human hepatocytes in a 1:1 total mRNA:total gRNA weight ratio.

FIG. 3 shows dose response curves in human hepatocellular carcinoma immortalized cells (HuH-7) for five (5) pairs of first and second guide RNAs using a gene editing system that comprises a dual nickase Cas9 system as described in connection with FIG. 2. The Cas9 nickase is encoded within an mRNA (MS029) that was transfected into the HuH-7 cells in a 1:1 total mRNA: total gRNA weight ratio. As illustrated in FIG. 3, each of the 5 guide oligonucleotide pairs showed an escalating dose response with increasing concentrations.

FIG. 4A is a schematic drawing illustrating the LPA genetic sequence to which spacers of guides GA1183, GA1184, GA1264, and GA1266 correspond. The corresponding amino acid sequence is provided below the genetic sequence and is numbered. Guide pairs (GA1183/GA1184, GA1264/GA1184, GA1266/GA1184) for dual nicking of the LPA gene are shown. The guide pairs were formulated into lipid nanoparticles (LNPs) with SpCas9-D10A nickase mRNA (MS029) and tested for editing efficiency, with LNP 1 corresponding to guide pair GA1183/GA1184, LNP 2 corresponding to guide pair GA1264/GA1184, and LNP 3 corresponding to guide pair GA1266/GA1184. Results are shown in FIG. 4B.

FIG. 4B is a plot illustrating editing efficiency (editing %) of primary human hepatocyte (PHH) cells incubated with LNPs (LNP 1, LNP 2, LNP 3 as described in the brief description of FIG. 4A) in a dose responsive manner ranging from 0 to 40,000 ng/mL of total RNA (guides and mRNA). As illustrated in FIG. 4B, the gene editing system (guide pairs and mRNA encoding SpCas9-D10A nickase) resulted in an escalating response with increasing concentrations.

FIG. 5 is a plot showing percent of apo(a) protein secreted from an HuH-7 reporter cell line exposed to varying concentrations of LNP ranging from 0 to 5000 ng/mL (total RNA). The LNP corresponds to LNP 1 as described in the brief description of FIGS. 4A-B and includes the GA1183/GA1184 guide pair and mRNA encoding the SpCas9-D10A nickase (MS029). Apo(a) protein concentration was assessed using a validated Lp(a) ELISA kit from Mercodia. The HuH-7 reporter cell line was generated by (i) infection with lentiviruses containing an expression cassette comprising an LPA open reading frame (ORF) followed by an internal ribosome entry site (IRES) and puromycin N-acetyltransferase (puro), driven by a cytomegalovirus (CMV) promoter, and (ii) selection with puromycin. As illustrated in FIG. 5, the relative percent reduction in secreted apo(a) protein from the reporter cell line was reduced in a LNP dose-dependent manner.

FIGS. 6A and 6B are graphs of LPA gene editing efficiency (indel %) in livers harvested from transgenic mice expressing the human LPA gene fourteen days after administration of a gene editing system (guide GA1296, guide GA1295, and MS029 mRNA). The gene editing system was formulated into lipid nanoparticles (LNPs) and delivered to the transgenic mice via retro-orbital injection at various concentrations. Concentrations shown in FIGS. 6A and 6B correspond to mg total RNA (guide pair and mRNA) per kilogram (based on mouse weight). As illustrated in FIGS. 6A and 6B, gene editing efficiency increased in a dose-dependent manner with increasing dose of the gene editing system.

FIG. 7 is a graph of percent change in plasma apo(a) protein level (measured as a percent change from baseline) in transgenic mice expressing the human LPA gene fourteen days after administration of a gene editing system (guide GA1296, guide GA1295, and MS029 mRNA). The gene editing system was formulated into lipid nanoparticles (LNPs) and delivered to the transgenic mice via retro-orbital injection at various concentrations. Concentrations shown in FIG. 7 correspond to mg total RNA (guide pair and mRNA) per kilogram (based on mouse weight). Plasma apo(a) concentrations were determined at baseline (−7 days) and 14 days following administration of the gene editing system. As illustrated in FIG. 7, percent reduction in plasma apo(a) protein levels increased in a dose-dependent manner with increasing dose of the gene editing system.

FIG. 8A is a graph of change in plasma apo(a) protein levels (measured as a percent change from baseline) in transgenic mice expressing the human LPA gene 7 and 14 days after administration of a gene editing system (guide GA1296, guide GA1295, and MS029 mRNA). The gene editing system was formulated into lipid nanoparticles (LNPs) and delivered to the transgenic mice via retro-orbital injection at various concentrations. Concentrations shown in FIG. 8A correspond to mg total RNA (guide pair and mRNA) per kilogram (based on mouse weight). Plasma apo(a) concentrations were determined at baseline (−7 days) and 7 days and 14 days following administration of the gene editing system. As illustrated in FIG. 8A, percent reduction in plasma apo(a) protein levels increased in a dose-dependent manner with increasing dose of the gene editing system.

FIG. 8B is a graph of change in plasma apo(a) protein levels (measured as a percent change from baseline) in transgenic mice expressing the human LPA gene 14 days after administration of various gene editing systems (guide oligonucleotide pairs and MS029 mRNA). The gene editing system was formulated into lipid nanoparticles (LNPs) and delivered to the transgenic mice via retro-orbital injection at a 0.5 mg/kg dose. The dose corresponds to mg total RNA (guide pair and mRNA) per kilogram (based on mouse weight). Plasma apo(a) concentrations were determined at baseline (−7 days) and 14 days following administration of the gene editing systems.

FIG. 9 is a diagrammatic representation showing the locations at which the spacers of selected gRNAs described herein are complementary to the LPA gene and thereby are capable of hybridizing thereto. Due to the repetitive nature of some sequences of the LPA gene (e.g., the kringle type IV repeats), the spacers of some of the selected gRNAs are complementary to more than one location in the LPA gene.

### DETAILED DESCRIPTION OF ILLUSTRATIVE EMBODIMENTS

Provided herein, among other things, are compounds and compositions for LPA gene modification or editing and methods of using the same. In embodiments, the methods result in reduction of blood Lp(a) concentrations by inactivating the LPA gene. In embodiments, editing of the LPA gene results in indel variants and non-synonymous variants in the LPA sequence. Compositions and methods directed to editing the LPA gene using an editing system that can install the edit, such as a Cas nickase (e.g., a Cas9 nicakse) and two guide oligonucleotides (e.g., gRNAs), are disclosed.

For convenience, this Detailed Description is arranged in the following sections:
I. DEFINITIONS
II. APOLIPOPROTEIN(A) PROTEIN AND LPA GENE
III. GENE EDITING/GENE MODIFICATION
IV. GUIDE NUCLEIC ACIDS AND TARGETED DNA SEQUENCES
V. GENE EDITOR SYSTEMS
VI. THERAPEUTIC APPLICATIONS
VII. PHARMACEUTICAL COMPOSITIONS
   A. Lipid Nanoparticle (LNP) Compositions
      1. Amino Lipids
         a) Formula (I)
         b) Formula (Ia)
         c) Variations of Formula (I) and (Ia)
      2. LNP Compositions Comprising Different Amino Lipids
      3. Additional Amino Lipid Embodiments
      4. PEG-Lipids
      5. Phospholipid
      6. Cholesterol
      7. GalNAc-Lipid
      8. Phosphate charge neutralizer
      9. Antioxidants
      10. Other Lipids
      11. LNP Formulation
      12. Payload
VIII. KITS
IX. DOSING
X. MEANS
XI. EXAMPLES
XII. OTHER EMBODIMENTS

### I. DEFINITIONS

The following presents definitions of some terms presented throughout this disclosure. In some instances, terms are defined in areas of this specification other than in this “Definitions” section.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use. The use of the term “or” implies that the disjunctive sense is contemplated. That is, while “A, B, or C” may mean “A, B, and/or C” or “A, B, C, or any combination thereof,” “A, B, or C” will also include “A or B or C, but not (A and B), (A and C), (B and C), and (A, B, and C).”

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated, the term “about” means within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

An article, composition, method, or the like that comprises one or more elements may consist of the one or more elements or may consist essentially of the one or more elements. As used in this specification and claim(s), “consisting of” (and any form of consisting of, such as “consists of” and “consist of”) means including and limited to. As used in this specification and claim(s), an article, composition, method, or the like “consisting essentially of” (and any form of consisting essentially of, such as “consists essentially of” and “consist essentially of”) means the article, composition, method, or the like includes the specified enumerated elements; such as components, compounds, materials, steps, or the like, and may include additional elements that do not materially affect the basic and novel characteristics of the article, composition, method, or the like.

Reference in the specification to “some embodiments,” “an embodiment,” “one embodiment,” “one or more embodiments,” “embodiments” or “other embodiments” means that a particular feature, structure, or characteristic described in connection with the embodiments is included in at least one or more embodiments, but not necessarily all embodiments, of the present disclosure. To the extent that the disclosure describes aspects, components, or elements associated with a particular embodiment in more detail or breadth, it is contemplated that the aspects, components, or elements associated with such embodiment should be understood to encompass the additional detail and breadth described in the disclosure.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine (“A”), thymine (“T”), guanine (“G”), cytosine (“C”), uracil (“U”), inosine (“I”), and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages or modified sugar residues, or non-canonical/chemically-modified nucleobases and combinations thereof, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide (e.g., gRNA) or polynucleotide (e.g., mRNA, genomic DNA), with fragments containing up to 150 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, for example, antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases. It should be understood that the terms “polynucleotide” and “oligonucleotide” can also include polymers or oligomers comprising both deoxy and ribonucleotide combinations or variants thereof in combination with backbone modifications, such as those described herein.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The nucleic acid described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) prevents nuclease recognition and degradation. Backbone modifications can also include peptide bonds instead of phosphorous in the backbone structure (e.g., N-(2-aminoethyl)-glycine units linked by peptide bonds in a peptide nucleic acid) or linking groups including carbamate, amides, and linear and cyclic hydrocarbon groups. Oligonucleotides with modified backbones are reviewed in Micklefield, Curr. Med. Chem., 8 (10): 1157-79, 2001 and Lyer et al., Curr. Opin. Mol. Ther., 1 (3): 344-358, 1999. Nucleic acid molecules described herein may contain a sugar moiety that comprises ribose or deoxyribose, as present in naturally occurring nucleotides, or a modified sugar moiety or sugar analog. Modified sugar moieties include, but are not limited to, 2′-O-methyl, 2′-O-methoxyethyl, 2′-O-aminoethyl, 2′-Fluoro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, and bicyclic modified sugars. 2′-O-methyl or 2′-0-methoxyethyl modifications may be included to promote the “A-form” or “RNA-like” conformation in oligonucleotides, increase binding affinity to RNA, and enhance nuclease resistance. Modified sugar moieties can also include an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

The present disclosure encompasses man-made, isolated, or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples of proteins include hormones, enzymes, antibodies, and any fragments thereof. In some cases, a protein can be a portion of the protein, for example, a domain, a subdomain, or a motif of the protein. In some cases, a protein can be a variant (or mutant) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material are well known in the art and include, but are not limited to, Western blotting, flow cytometry, enzyme-linked immunosorbent assay (ELISA), radioimmunoassay (RIA), and various proteomics techniques, such as mass spectrometry. An exemplary method to measure or detect a polypeptide is an immunoassay, such as an ELISA. This type of protein quantitation can be based on an antibody capable of capturing a specific antigen, and a second antibody capable of detecting the captured antigen.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs; and the like.

“A subject in need thereof” refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In one or more embodiments, the subject has an elevated blood Lp(a) concentration.

“Administering” and its grammatical equivalents as used herein can refer to providing one or more drug substances (e.g., mRNA that encodes the editor proteins, guide oligonucleotides), drug products (e.g., LNPs that encapsulate the drug substances for delivery to the target cells/tissue), or pharmaceutical compositions thereof as described herein to a subject or a patient. By way of example and without limitation, “administering” can be performed by intravenous (i.v.) injection, subcutaneous (s.c.) injection, intradermal (i.d.) injection, intraperitoneal (i.p.) injection, intramuscular (i.m.) injection, intravascular injection, intracerebroventricular (i.e.v.) injection, intrathecal (i.t.) injection, infusion (inf.), oral routes (p.o.), topical (top.) administration, or rectal (p. r.) administration. One or more such routes can be employed.

The term “parenteral” as used herein includes subcutaneous, intracutaneous, intravenous, intramuscular, intraarticular, intraarterial, intrasynovial, intrasternal, intracerebroventricular, intrathecal, intralesional, and intracranial injection or infusion techniques. Parenteral administration can be, for example, by bolus injection or by gradual perfusion over time. In addition, it can be administered to the subject via injectable depot routes of administration such as using 1-, 3-, or 6-month depot injectable or biodegradable materials and methods.

The terms “treat,” “treating,” or “treatment,” and their grammatical equivalents as used herein, can include alleviating, abating, or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. “Treating” may refer to administration of a composition comprising a nanoparticle, such as a lipid nanoparticle (LNP), to a subject before or after the onset, or suspected onset, of a disease or condition. “Treating” includes the concepts of “alleviating,” which refers to lessening the frequency of occurrence or recurrence, or the severity, of any symptoms or other ill effects related to a disease or condition and/or the side effects associated with the disease or condition. The term “treating” also encompasses the concept of “managing,” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention.” It is appreciated that, although not precluded, treating a disorder or condition does not require that the disorder, condition, or symptoms associated therewith be completely eliminated.

As used herein, the terms “prevent,” “preventing,” “prevention,” and the like refer to reducing the probability of developing a disease or condition in a subject, who does not have but is at risk of or susceptible to developing a disease or condition.

The term “ameliorate” as used herein can refer to decrease, suppress, attenuate, diminish, arrest, or stabilize the development or progression of a disease.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence.

The term “therapeutic agent” or “drug substance” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids such as guide oligonucleotides and mRNA.

The term “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier or diluent, that does not abrogate the biological activity or properties of the compound and is relatively nontoxic, i.e., the material may be administered to a subject without causing undesirable biological effects or interacting in a deleterious manner with any of the components of the pharmaceutical composition in which it is contained.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985).

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “complementary” is used throughout this application to describe two related nucleic acid sequences that may form a double-stranded complex of a first 5′ to 3′ “top” strand and a second 3′ to 5′ “bottom” strand. A “spacer” sequence of a guide nucleic acid is considered to be “complementary” to a sequence of a target nucleic acid. In the context of a guide oligonucleotide, a sequence may be considered sufficiently “complementary” to a target sequence if it is capable of sufficiently hybridizing to the intended DNA strand so that it may be used to guide an editor protein to the target sequence to cause an intended edit. Thus, a guide oligonucleotide is complementary if, for example, it is capable of sufficiently hybridizing to the intended DNA strand so that it operationally positions the editor in the desired location to facilitate the intended edit.

As used herein, a nucleic acid sequence that is “substantially identical” to another nucleic acid sequence is a nucleotide sequence that has 70% or more sequence identity to the other nucleic acid sequence. In some embodiments, a nucleic acid sequence that is “substantially identical” to another nucleic acid sequence has 75% or more, 80% or more, 85% or more, 90% or more, 91% or more, 92% or more, 93% or more, 94% or more, 95% or more, 96% or more, 97% or more, 98% or more, or 99% or more sequence identity to the other nucleic acid sequence.

For purposes of percent sequence identity between an RNA sequence and a DNA sequence, uracil bases in the RNA are to be considered identical to thymine bases in the DNA.

As used herein “sequence identity” refers to the extent to which two optimally aligned nucleic acid sequences are invariant throughout a window of alignment of components, e.g., nucleotides. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part 1 (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) nucleic acid (or its complementary strand) as compared to a test (“subject”) nucleic acid (or its complementary strand) when the two sequences are optimally aligned. Percent sequence identity may be determined, when the compared sequences are aligned for maximum correspondence, as measured using a sequence comparison algorithm described below and as known in the art, or by visual inspection.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). Alignment of sequences may be analyzed using a Burrows-Wheeler transform such as BOWTIE open-source software available from https://github.com/BenLangmead/bowtie. An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100.

“Percent identity” may also be determined using BLASTX version 2.0 for translated nucleotide sequences and BLASTN version 2.0 for polynucleotide sequences. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands.

In addition to calculating percent sequence identity, the BLAST algorithm also performs a statistical analysis of the similarity between two sequences (see, e.g., Karlin & Altschul, Proc. Natl. Acad. Sci. U.S.A 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001.

Sequence “identity” (or, e.g., “or identicality” or “identical to”), as used herein, refers to the amount of nucleotide or amino acid which match exactly between two different sequences. When comparing RNA and DNA sequences Uracil and Thymine bases are considered to be the same base. Gaps are not counted, and the measurement is typically in relation to the shorter of the two sequences.

For example, for nucleotide sequences: A: AAGGCTT; B: AAGGC; and C: AAGGCAT, identity of sequence A compared to reference sequence B ((Identity(A, B)) is 100% (5 identical nucleotides/min(length(A), length(B))); Identity of sequence B to reference sequence C is 100%; but identity of sequence A to reference sequence C is 85% ((6 identical nucleotides/7).

Sequence “similarity” (or, e.g., “similar” sequences), as used herein, can be described as an optimal matching problem that finds the minimal number of edit operations (inserts, deletes, and substitutions) in order to transform a first sequence into an exact copy of a second sequence being aligned (edit distance). If the lengths of the first and second sequences are different, similarity is determined relative to the length of the shortest sequence. That is, sequence similarity is [1-(number of edit operations/length of the shortest sequence)]. Using this, the percentage sequence similarity for nucleotide sequences A: AAGGCTT; B: AAGGC; and C: AAGGCAT, similarity of sequence A and sequence B is 60%, similarity of sequence B and sequence C is 60%, and similarity of sequence A and sequence C is 86%.

It should be understood that comparisons of sequences may be determined including or excluding chemical modifications. Thus, when comparing a modified oligonucleotide to a reference unmodified oligonucleotide, percent sequence identity is determined based on the sequences of nucleobases in the modified oligonucleotide, where modified nucleobases are considered equivalent to unmodified nucleobases from which the modified nucleobases are derived. For example, 2,4,-Dichlorotoluene, 2,4-Dibromotholuene, and 2,4-Diiodotolume are considered equivalent to Thymine for purposes of comparing sequence identity of a modified oligonucleotide to a reference unmodified oligonucleotide. As another example, 4-methylbenzimidazole and 9-methylimidazo are considered equivalent to Adenine for purposes of comparing sequence identity of a modified oligonucleotide to a reference unmodified oligonucleotide. If the modified oligonucleotide comprises modifications to the linkage moiety (e.g., other than phosphate linker) and/or to the sugar (e.g., 2′ modified ribose or dideoxyribose), then the sequence of the nucleobases of the modified oligonucleotides is compared to the sequence of the nucleobases in the reference unmodified sequence to determine sequence identity. For example, for nucleotide sequences: A: mA*AGGCmT*mT; B: AAGGCTT; and C: AAGGCAT, identity of sequence A to reference sequence B is 100% (7 identical nucleotides/7); identity of sequence A to reference sequence C is 85% (6 identical nucleotides/7). The percentage sequence similarity of the examples above are: Similarity of sequences A and B is 100% and sequences A and C is 86%.

When chemical modifications are included when determining identicality, sequence identity is determined based on the exact modifications of each nucleotide of the reference modified oligonucleotide (e.g., based on any modifications to the linker, the sugar moiety, and the nucleobase). Accordingly, an unmodified (or differently modified) nucleotide of an oligonucleotide is not considered identical to a corresponding modified oligonucleotide of a reference modified oligonucleotide. Thus, for example, when the following two sequences are compared for purposes of identicality: A: AAGGCTTC; B: mA*AGGCmT*mT, identity of sequence A to reference sequence B is 57% (4 identical nucleotides/7), when chemical modifications are not excluded; and identity of sequence A to reference sequence B is 100% (7 identical nucleotides/7), when chemical modifications are excluded.

Comparison of any modified sequence can be determined with or without chemical modifications being considered if so stated, for example, in a claim as such.

As used herein, a spacer sequence of a guide nucleic acid is considered to be “homologous” to a protospacer sequence of a target nucleic acid if a gene editor system comprising a guide oligonucleotide having the spacer sequence is capable of making a modification (e.g., nick) to the target nucleic acid (e.g., nick on sense strand or antisense strand of the target nucleic acid). A spacer sequence that is homologous to a protospacer sequence may be identical or substantially identical to the protospacer sequence.

In some embodiments, a first nucleotide sequence that is homologous to a second nucleotide sequence may hybridize to the complementary sequence of the second nucleotide sequence under stringent conditions or highly stringent conditions. “Stringent hybridization conditions” and “stringent hybridization wash conditions” in the context of nucleic acid hybridization are sequence-dependent and are different under different environmental parameters. An extensive guide to the hybridization of nucleic acids is found in Tijssen, Laboratory Techniques in Biochemistry and Molecular Biology-Hybridization with Nucleic Acid Probes part 1 chapter 2 “Overview of principles of hybridization and the strategy of nucleic acid probe assays” Elsevier, New York (1993). Generally, highly stringent hybridization and wash conditions are selected to be about 5 °C lower than the thermal melting point Tm for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42 °C, with the hybridization being carried out overnight. An example of highly stringent wash conditions is 0.15M NaCl at 72 °C for about 15 minutes. An example of stringent wash conditions is a 0.2×SSC wash at 65 °C for 15 minutes (see, Sambrook and Russel, Molecular Cloning: A Laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1×SSC at 45 °C for 15 minutes. An example of a low stringency wash for a duplex of, e.g., more than 100 nucleotides, is 4-6×SSC at 40 °C for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and a temperature typically at least about 30 °C. Stringent conditions can also be achieved with the addition of destabilizing agents such as formamide.

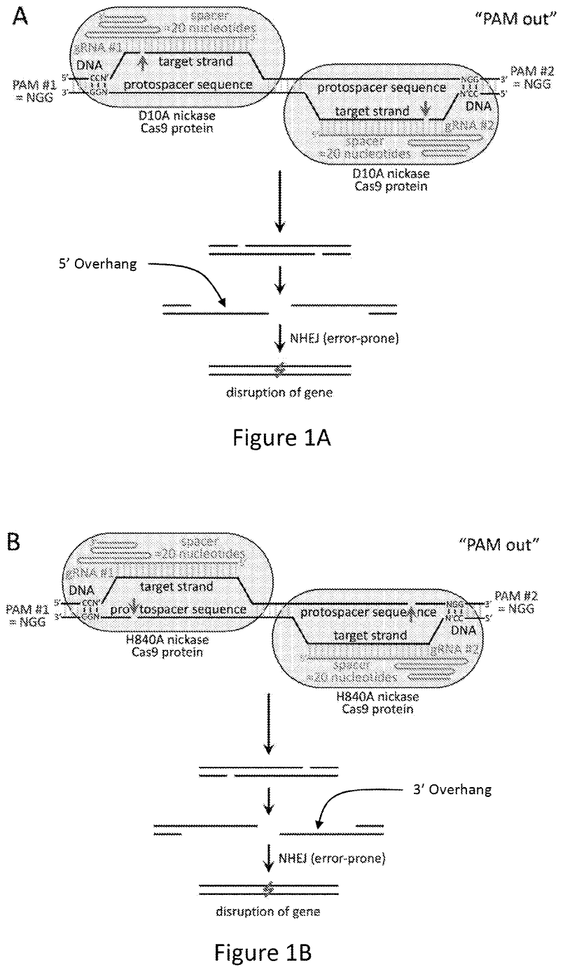

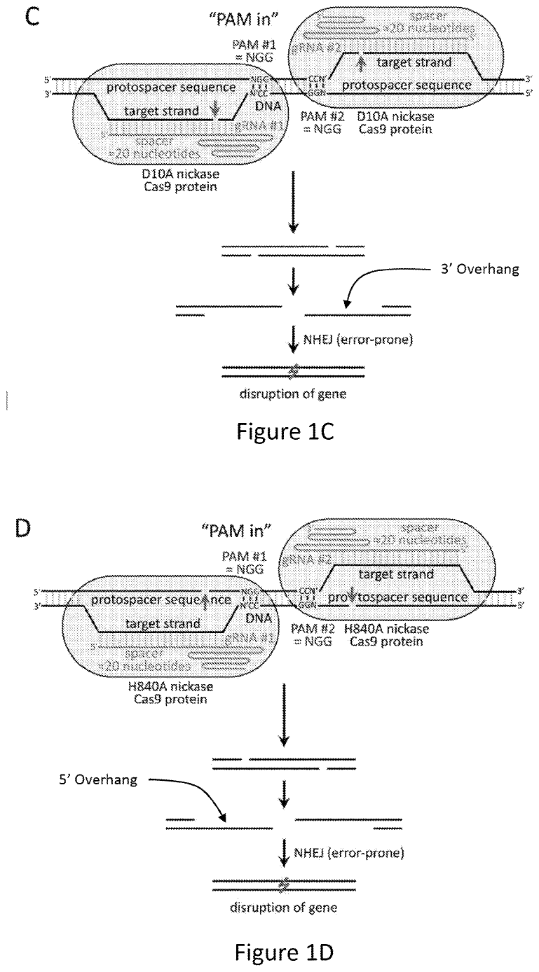

The term “effect” or “effectuate” and their grammatical equivalents are used throughout this disclosure to refer to an action made that will induce creation of an intended result. The entire process by which the result is produced may not necessarily be performed by the system used to effect the edit. Cellular processing may, for example, play a role in completing the intended result. However, the intended result would not occur without the system. That is, the system is necessary for the intended result to be effected. For example, a dual nickase in vivo editor system such as those illustrated in FIGS. 1A-1D that include a Cas9 nickase and two guide oligonucleotides may effect an edit in a gene without performing final processing steps that result in the edit being fully incorporated in both strands of the gene. Cellular DNA repair enzymes may perform the final processing steps. While all the steps needed to complete the edit would not be performed by the dual nickase editor system, the system would still be said to have effected the edit.

As used herein “inactivating the LPA gene” and grammatical equivalents thereof refer to edits to the LPA gene by a gene editing system that result in decreased expression of apo(a), expression of a non-functional variant of apo(a), and/or expression of an apo(a) variant having reduced function relative to wild-type apo(a). The edits to the LPA gene effected by the gene editing systems described herein may result in indel and/or non-synonymous variants. The indel and/or non-synonymous variants of the LPA gene may result in production of pre-mRNA that is degraded via, for example, nonsense-mediated decay. The indel and/or non-synonymous variants of the LPA gene may result in production of apo(a) variants having reduced function, and in some cases no function, relative to wild-type apo(a).

In several places throughout the application, guidance is provided through examples, which examples, including the particular aspects thereof, can be used in various combinations and be the subject of claims. In each instance, the recited elements serve only as a representative group and should not be interpreted as an exclusive list. It is to be understood that the particular examples, materials, amounts, and procedures are to be interpreted broadly in accordance with the scope and spirit of the invention as set forth herein, and it is contemplated that the various aspects set forth in the examples and the disclosure may be combined and set forth in patentably distinct claims.

For any method disclosed herein that includes discrete steps, the steps may be conducted in any feasible order, and, as appropriate, any combination of two or more steps may be conducted simultaneously.

### II. APOLIPOPROTEIN(A) PROTEIN AND LPA GENE

The LPA gene is highly similar to the PLG (plasminogen) gene, from which it evolved in primates, and the protein products of the two genes have structural commonalities. They each have kringle type IV (KIV) and kringle type V (KV) domains and protease-like domains. Apo(a) comprises 10 types of KIV domains (KIV₁ through KIV₁₀), with a variable number of repeat KIV₂ domains ranging from 2 to more than 40, and single domains of the other 9 types, a single KV domain, and an inactive protease-like domain. Blood Lp(a) concentrations are largely genetically determined and vary inversely with the number of KIV₂ repeats, as LPA alleles with fewer repeats result in smaller apo(a) isoforms that are more quickly produced and processed in hepatocytes. There is broad inter-individual variation in blood Lp(a) concentrations, with one in five individuals having elevated concentrations that signify substantially increased risk of cardiovascular disease. Conversely, individuals with very low blood Lp(a) concentrations, caused by naturally occurring null variants in LPA, are protected against ASCVD without any major adverse consequences.

Apolipoprotein(a) [apo(a)] is a defining component of Lp(a). Apo(a) is understood to be primarily synthesized in the hepatocyte cells of the liver. Apo(a) comprises single copies of the KIV₁ domain and KIV₃ through KIV₁₀ domains, a variable number of repeat KIV₂ domains ranging from 2 to more than 40, a single KV domain, and an inactive protease-like domain. The wild-type apo(a) protein that is annotated in the reference human genome (GRCh38/hg38, NCBI) has six repeat KIV₂ domains, is 2040 amino acids in length, and has the amino acid sequence below. It should be understood that the sequence below represents one of many isoforms of apo(a) present in the human population.

MEHKEVVLLLLLFLKSAAPEQSHVVQDCYHGDGQSYRGTYSTTVTGRTCQ
AWSSMTPHQHNRTTENYPNAGLIMNYCRNPDAVAAPYCYTRDPGVRWEYC
NLTQCSDAEGTAVAPPTVTPVPSLEAPSEQAPTEQRPGVQECYHGNGQSY
RGTYSTTVTGRTCQAWSSMTPHSHSRTPEYYPNAGLIMNYCRNPDAVAAP
YCYTRDPGVRWEYCNLTQCSDAEGTAVAPPTVTPVPSLEAPSEQAPTEQR
PGVQECYHGNGQSYRGTYSTTVTGRTCQAWSSMTPHSHSRTPEYYPNAGL
IMNYCRNPDAVAAPYCYTRDPGVRWEYCNLTQCSDAEGTAVAPPTVTPVP
SLEAPSEQAPTEQRPGVQECYHGNGQSYRGTYSTTVTGRTCQAWSSMTPH
SHSRTPEYYPNAGLIMNYCRNPDAVAAPYCYTRDPGVRWEYCNLTQCSDA
EGTAVAPPTVTPVPSLEAPSEQAPTEQRPGVQECYHGNGQSYRGTYSTTV
TGRTCQAWSSMTPHSHSRTPEYYPNAGLIMNYCRNPDAVAAPYCYTRDPG
VRWEYCNLTQCSDAEGTAVAPPTVTPVPSLEAPSEQAPTEQRPGVQECYH
GNGQSYRGTYSTTVTGRTCQAWSSMTPHSHSRTPEYYPNAGLIMNYCRNP
DAVAAPYCYTRDPGVRWEYCNLTQCSDAEGTAVAPPTVTPVPSLEAPSEQ
APTEQRPGVQECYHGNGQSYRGTYSTTVTGRTCQAWSSMTPHSHSRTPEY
YPNAGLIMNYCRNPDAVAAPYCYTRDPGVRWEYCNLTQCSDAEGTAVAPP
TVTPVPSLEAPSEQAPTEQRPGVQECYHGNGQSYRGTYSTTVTGRTCQAW
SSMTPHSHSRTPEYYPNAGLIMNYCRNPDPVAAPYCYTRDPSVRWEYCNL
TQCSDAEGTAVAPPTITPIPSLEAPSEQAPTEQRPGVQECYHGNGQSYQG
TYFITVTGRTCQAWSSMTPHSHSRTPAYYPNAGLIKNYCRNPDPVAAPWC
YTTDPSVRWEYCNLTRCSDAEWTAFVPPNVILAPSLEAFFEQALTEETPG
VQDCYYHYGQSYRGTYSTTVTGRTCQAWSSMTPHQHSRTPENYPNAGLTR
NYCRNPDAEIRPWCYTMDPSVRWEYCNLTQCLVTESSVLATLTVVPDPST
EASSEEAPTEQSPGVQDCYHGDGQSYRGSFSTTVTGRTCQSWSSMTPHWH
QRTTEYYPNGGLTRNYCRNPDAEISPWCYTMDPNVRWEYCNLTQCPVTES
SVLATSTAVSEQAPTEQSPTVQDCYHGDGQSYRGSFSTTVTGRTCQSWSS
MTPHWHQRTTEYYPNGGLTRNYCRNPDAEIRPWCYTMDPSVRWEYCNLTQ
CPVMESTLLTTPTVVPVPSTELPSEEAPTENSTGVQDCYRGDGQSYRGTL
STTITGRTCQSWSSMTPHWHRRIPLYYPNAGLTRNYCRNPDAEIRPWCYT
MDPSVRWEYCNLTRCPVTESSVLTTPTVAPVPSTEAPSEQAPPEKSPVVQ
DCYHGDGRSYRGISSTTVTGRTCQSWSSMIPHWHQRTPENYPNAGLTENY
CRNPDSGKQPWCYTTDPCVRWEYCNLTQCSETESGVLETPTVVPVPSMEA
HSEAAPTEQTPVVRQCYHGNGQSYRGTFSTTVTGRTCQSWSSMTPHRHQR
TPENYPNDGLTMNYCRNPDADTGPWCFTMDPSIRWEYCNLTRCSDTEGTV
VAPPTVIQVPSLGPPSEQDCMFGNGKGYRGKKATTVTGTPCQEWAAQEPH
RHSTFIPGTNKWAGLEKNYCRNPDGDINGPWCYTMNPRKLFDYCDIPLCA
SSSFDCGKPQVEPKKCPGSIVGGCVAHPHSWPWQVSLRTRFGKHFCGGTL
ISPEWVLTAAHCLKKSSRPSSYKVILGAHQEVNLESHVQEIEVSRLFLEP
TQADIALLKLSRPAVITDKVMPACLPSPDYMVTARTECYITGWGETQGTF
GTGLLKEAQLLVIENEVCNHYKYICAEHLARGTDSCQGDSGGPLVCFEKD
KYILQGVTSWGLGCARPNKPGVYARVSRFVTWIEGMMRNN.

It should be understood that the foregoing sequence employs standard conventional nomenclature well known in the art with each letter denoting an amino acid.

| Letter | 3 Letter | Amino Acid |
|---|---|---|
| A | Ala | alanine |
| B | Asx | aspartic acid or asparagine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| V | Val | valine |
| W | Trp | tryptophan |
| Y | Tyr | tyrosine |
| Z | Glx | glutamic acid or glutamine |

The LPA gene that is annotated in the reference human genome (GRCh38/hg38, NCBI) is composed of 39 exons and is located on chromosome 6 at 6q25.3 from nucleotide 160,664,275 to nucleotide 160,531,482.

The full sequence of the human LPA gene is available at UniProtKB—P08519 (APOA_HUMAN).

Elevated blood Lp(a) concentrations are a well-established casual risk factor for ASCVD and for calcific aortic valve disease. Individuals with very low blood Lp(a) concentrations, caused by naturally occurring null variants in LPA, are protected against ASCVD without any major adverse consequences.

The inventors show below that editing to introduce indel or non-synonymous variants in the coding sequence of the LPA gene using a gene editing approach is possible, providing a one-time treatment approach for patients with elevated blood Lp(a) concentrations.

Any subject having an elevated blood Lp(a) concentration may be treated using the gene editing approach described herein. Subjects having elevated blood Lp(a) concentrations may be identified through laboratory testing, genetic screening, or the like, or a combination thereof.

In embodiments, a method includes identifying a subject having an elevated blood Lp(a) concentration and administering to the subject a composition comprising components capable of introducing indel or non-synonymous variants in the coding sequence of the LPA gene. Administering the composition to the subject may comprise administering a therapeutically effective amount of the composition to the subject. The effected edits of the LPA gene may result in reduced functionality of the apo(a) protein. The effected edits of the LPA gene may result in a reduction of the blood Lp(a) concentration.

The compositions described herein that include a gene editing system include one or more guide nucleic acids designed to target one or more protospacer regions on the LPA gene.

### III. GENE EDITING/GENE MODIFICATION

The term “gene editing” or “gene modification” and their grammatical equivalents as used herein refer to genetic engineering in which one or more nucleotides are modified, inserted, replaced, or removed from a genome. Gene editing can be performed using man-made non-naturally occurring gene editing systems that comprise one or more nucleases, which may be derived from naturally existing nucleases or artificially engineered. Gene modification can include introducing a double-strand break, a nonsense variant, a frameshift variant, a splice site alteration, or an inversion in a polynucleotide sequence, e.g., a target polynucleotide sequence. Gene modification can also be accomplished using other editing systems, such as dual nickase editor systems such as those illustrated and described in FIGS. 1A-1D.

Any suitable gene editing system may be employed to effect the editing in the LPA gene. The gene editing system may include a gene editor and a suitable guide nucleic acid for use with the gene editor. For purposes of the present disclosure, a “gene editor system” that includes a nucleic acid capable of being translated, or transcribed and translated, into a gene editor protein or protein component of a gene editor is considered to be a gene editor system that includes a gene editor. The nucleic acid may be, for example, a plasmid DNA or an mRNA. The mRNA may be mature mRNA or pre-mRNA that may be processed, e.g., within a cell, to mature mRNA, which then may be translated, e.g., within a cell, to produce the gene editor. For purposes of the present disclosure, a “gene editor system” that includes a nucleic acid capable of being transcribed to produce a gRNA is considered to be a gene editor system that comprises the gRNA. The nucleic acid may be, for example, a plasmid DNA. In embodiments, the gene editor is a Cas9 variant or related gene editor. In embodiments, the gene editor is a nickase such as described in FIGS. 1A-1D.

### A. Cas9 and Related Nucleic Acid-Directed Editing Proteins

A ribonucleoprotein complex from S. pyogenes capable of introducing double-strand breaks in double-stranded DNA was published by Jennifer Doudna's group in 2012 (Jinek et al. Science 2012 Aug. 17; 337(6096):816-21). In this context, the term “ribonucleoprotein” is used to refer to a non-covalent assembly of RNA and protein. Clustered regularly interspaced short palindromic repeats (CRISPR) and a CRISPR-associated protein (Cas) was described. It was shown that the CRISPR regions can be transcribed into short sequences of RNA (crRNA), which can then form a hairpin with another short RNA sequence called a trans-activating CRISPR RNA (tracrRNA) to form a guide RNA (gRNA). Operationally, the crRNA comprises a nucleotide sequence (known as a “spacer” or “spacer sequence”) that is complementary to the target strand of the DNA, whereas the tracrRNA serves as a binding scaffold for the Cas protein. The Cas protein then may complex with the gRNA, and the ribonucleoprotein is targeted to a sequence substantially identical to the crRNA sequence. The sequence that is substantially identical to the crRNA is known as the “protospacer” sequence, and it is directly adjacent to a “protospacer-adjacent motif” (PAM). For S. pyogenes Cas9, the PAM is an “NGG” sequence, wherein N represents any standard nucleotide, and G represents guanine.

The Cas protein contains two nuclease domains capable of cutting the phosphate backbone of a nucleic acid, termed the HNH domain and the RuvC domain. The HNH domain cuts the backbone of the strand complementary to the gRNA, referred to as the “target strand”, while the RuvC domain cuts the backbone of the opposite strand, also referred to as the “non-target strand” or “displaced strand” (i.e., the strand that contains the protospacer). When the gRNA-Cas complex binds a sequence of double-stranded DNA, each nuclease domain cuts the backbone of one strand of the DNA, causing a double-strand break (DSB). The DSB may be repaired by the cell in several ways, including creation of an insertion or deletion (indel), homology-directed repair (HDR), microhomology-mediated end joining (MMEJ), or mismatch repair (MMR). Multiple Cas-based systems have been engineered to exploit each of these repair pathways. Guide RNA design has been further characterized and engineered to improve editing efficiency and yield different editing results.

Other variants of Cas9 and Cas-type proteins have also been characterized. Additionally, the amino acid sequence of each nuclease domains of the Cas9 may be mutated to impair the nuclease activity of the Cas9 protein to yield a “nickase” Cas9 protein (nCas9) that introduces a single-strand cut in a targeted DNA sequence, rather than a double-strand break. “D10A” mutation of the RuvC domain results in a Cas9 protein that will cut only the strand complementary to the gRNA (i.e., the target strand). Such nickases based on the “D10A” mutation of the RuvC domain of the Cas9 protein are used in base editing systems to nick the target strand, which is the strand opposite the strand that contains the protospacer. “H840A” mutation of the HNH domain results in a Cas9 protein that will cut only the displaced strand (i.e., the strand that contains the protospacer sequence). Such nickases based on the “H840A” mutation of the HNH domain of the Cas9 protein are used in template-based editing systems to nick the non-target strand or protospacer sequence. Mutations that impair both nuclease domains yield a catalytically dead Cas9 (also called “dCas9”), which is still capable of binding to a gRNA and targeting a region of a gene but does not inherently alter the target region. A variety of modified and unmodified Cas proteins have been described (see, e.g., Cong et al., Science 339, 819-823 (2013); Mali et al., Science 339, 823-826 (2013); Hwang et al., Nature Biotechnology 31, 227-229 (2013); Jinek et al., eLife 2, e00471 (2013); Dicarlo et al., Nucleic Acids Research (2013); and Jiang et al., Nature Biotechnology 31, 233-239 (2013)). Cas9 and Cas-type proteins include, but are not limited to, SpCas9 (e.g., dCas9 and nCas9), SaCas9 (e.g., SaCas9d, SaCas9d, SaKKH Cas9), NmeCas9, CasX, CasY, Cas12a/Cpf1, C2c1, C2c2, C2c3, and Argonaute. A gene editor described herein may include a Cas9 or Cas-related domain or protein or suitable variants of the domain or protein.

### B. Dual Nickase Editor Systems

A dual nickase gene editing system comprises one or more nickases, a first guide oligonucleotide, and a second guide oligonucleotide. At least one of the one or more nickases is configured to interact with the first guide oligonucleotide. In interaction with the first guide oligonucleotide, the nickase is engineered to nick one of a first or second strand of the LPA gene. At least one of the one or more nickases is configured to interact with the second guide oligonucleotide. In interaction with the second guide oligonucleotide, the nickase is engineered to nick the other of the first or second strands of the LPA gene. The nickase(s) that interacts with the first and second guide oligonucleotides may be the same or different. The resulting nicks on opposing strands in the LPA gene may activate a cellular DNA repair mechanism, which may result in an indel variant or non-synonymous variant in the LPA gene. Accordingly, the dual nickase system may effect the indel variant or the non-synonymous variant.

FIGS. 1A-ID illustrate dual nickase editing systems that comprise a single nickase editor. The nickase editor is a protein or protein complex capable of effecting indel variants or non-synonymous variants into a targeted DNA site using two guide nucleic acids. The nickase editor may comprise a nickase Cas9.

A dual nickase editor system (the nickase protein and two guide nucleic acids) may be capable of locating a specific target in a gene or genome, nicking both strands at separate positions at a targeted DNA site. The nickase protein may be catalytically inactivated or impaired such that it nicks or cuts at most one strand of a double-stranded nucleic acid target. The “D10A” mutation of the RuvC domain of the Cas9 protein described above and the “H840A” mutation of the HNH domain of the Cas9 protein described above are examples of an impaired nuclease that is capable of being used in a dual nickase editor system.

Any suitable dual nickase editor system may be used to effect editing in the LPA gene of a cell or subject. A cell or subject may be treated with an assembled lipid nanoparticle (LNP) encapsulating or otherwise comprising the nickase protein or a nucleic acid, such as an mRNA, encoding the nickase protein and two guide nucleic acids to effect the editing.

The dual nickase editing process may occur as shown in FIG. 1A or FIG. 1C. The targeted DNA site is engaged by the Cas9-D10A protein via the spacer region of the first guide nucleic acid, which is substantially identical to a protospacer sequence on one strand of the targeted DNA site. The Cas9-D10A domain nicks the target strand of the first guide nucleic acid (i.e., the strand hybridized to the first spacer sequence). Separately, the targeted DNA site is engaged by the Cas9-D10A protein via the spacer region of the second guide nucleic acid, which is substantially identical to a protospacer sequence on the other strand of the targeted DNA site. The Cas9-D10A domain nicks the target strand of the second guide nucleic acid (i.e., the strand hybridized to the second spacer sequence). The orientation of the protospacer sequences relative to each other dictates whether the two nick sites are some distance apart (FIG. 1A) or close together (FIG. 1C). The two nicks on opposing DNA strands are subsequently repaired by the cell via non-homologous end-joining and may yield indel or non-synonymous variants.

The dual nickase editing process may occur as shown in FIG. 1B or FIG. 1D. The targeted DNA site is engaged by the Cas9-H840A protein via the spacer region of the first guide nucleic acid, which is substantially identical to a protospacer sequence on one strand of the targeted DNA site. The Cas9-H840A domain nicks the non-target strand of the first guide nucleic acid (i.e., the strand with the first protospacer sequence). Separately, the targeted DNA site is engaged by the Cas9-H840A protein via the spacer region of the second guide nucleic acid, which is substantially identical to a protospacer sequence on the other strand of the targeted DNA site. The Cas9-H840A domain nicks the non-target strand of the second guide nucleic acid (i.e., the strand with the second protospacer sequence). The orientation of the protospacer sequences relative to each other dictates whether the two nick sites are close together (FIG. 1B) or some distance apart (FIG. 1D). The two nicks on opposing DNA strands are subsequently repaired by the cell via non-homologous end-joining and may yield indel or non-synonymous variants.

In some embodiments, the Cas9 nickase (or any other suitable nickase) in operation with the first and second guide nucleotides nicks opposing strands of the DNA to generate 5′ overhangs (e.g., as shown in FIGS. 1A and 1D). In some embodiments, the Cas9 nickase (or any other suitable nickase) in operation with the first and second guide nucleotides nicks opposing strands of the DNA to generate 3′ overhangs (e.g., as shown in FIGS. 1B and 1C). The overhangs may have any length suitable to allow non-homologous end-joining. It will be understood that the length of the overhangs suitable to allow non-homologous end-joining may vary based on, among other things, the composition of the overhangs. In some embodiments, the overhangs have a length from 1 to 200 nucleotides, such as 10 to 150 nucleotides, 15 to 100 nucleotides, or 20 to 50 nucleotides. In some embodiments, the overhangs have a length of 10 nucleotides or greater, such as 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or greater. In some embodiments, the overhangs have a length of 200 nucleotides or less, such as 190, 180, 170, 160, 150, 140, 130, 120, 110, 100, 90, 80, 70, 60, or 50 nucleotides or less. In some embodiments, the overhangs have a length of 20 to 50 nucleotides, such as 23 to 45, 30 to 40, 31 to 40, 32 to 40, 33 to 40, 31 to 39, 32 to 39, 33 to 39, or 34 to 38 nucleotides.

One implementation of a dual nickase editor system is the D10A mutation of the RuvC domain of the Streptococcus pyogenes Cas9 protein (SpCas9-D10A). The protein and cDNA sequences of SpCas9-D10A are shown in Table 1. Also shown in Table 1 is a chemically modified mRNA sequence (MS029) that has been engineered to express SpCas9-D10A in cells, including with respect to editing in the LPA gene as is further described herein.

One implementation of a dual nickase editor system is the H840A mutation of the RuvC domain of the Streptococcus pyogenes Cas9 protein (SpCas9-H840A). The protein and cDNA sequences of SpCas9-H840A are shown in Table 1. Also shown in Table 1 is a chemically modified mRNA sequence that has been engineered to express SpCas9-H840A in cells, including with respect to editing in the LPA gene as is further described herein.

TABLE 1
Nickase SpCas9 protein, cDNA, and mRNA sequences
SpCas9-D10A

| | | |
|---|---|---|
| Full | Protein | MDKKYSIGLAIGTNSVGWAVITDEYKVPSKKFKVLGNTDRHSIKKNLIGALLFDS |
| Sequence | | GETAEATRLKRTARRRYTRRKNRICYLQEIFSNEMAKVDDSFFHRLEESFLVEED |
| | | KKHERHPIFGNIVDEVAYHEKYPTIYHLRKKLVDSTDKADLRLIYLALAHMIKFR |
| | | GHFLIEGDLNPDNSDVDKLFIQLVQTYNQLFEENPINASGVDAKAILSARLSKSR |
| | | RLENLIAQLPGEKKNGLFGNLIALSLGLTPNFKSNFDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTFDNGSIPHQIHLGELHAILRRQEDFYPFLKDNREKIE |
| | | KILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLKRRRYTGW |
| | | GRLSRKLINGIRDKQSGKTILDFLKSDGFANRNFMQLIHDDSLTFKEDIQKAQV |
| | | SGQGDSLHEHIANLAGSPAIKKGILQTVKVVDELVKVMGRHKPENIVIEMARE |
| | | NQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPS |
| | | EEVVKKMKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYKVREINN |
| | | YHHAHDAYLNAVVGTALIKKYPKLESEFVYGDYKVYDVRKMIAKSEQEIGKATA |
| | | KYFFYSNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFDSPTVAYSV |
| | | LVVAKVEKGKSKKLKSVKELLGITIMERSSFEKNPIDFLEAKGYKEVKKDLIIKLPK |
| | | YSLFELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAENII |
| | | HLFTLTNLGAPAAFKYFDTTIDRKRYTSTKEVLDATLIHQSITGLYETRIDLSQLG |
| | | GDEGADKRTADGSEFESPKKKRKV* |
| | DNA | AGGACTCCTCCCCATCCTCTCCCTCTGTCCCTCTGTCCCTCTGACCCTGCACT |
| | | GTCCCAGCACCATGGACAAGAAGTACAGCATCGGCCTGGCCATCGGCACC |
| | | AACAGCGTGGGCTGGGCCGTGATCACCGACGAGTACAAGGTGCCCAGCAA |
| | | GAAGTTCAAGGTGCTGGGCAACACCGACCGCCACAGCATCAAGAAGAACC |
| | | TGATCGGCGCCCTGCTGTTCGACAGCGGCGAGACAGCCGAGGCCACCCGC |
| | | CTGAAGCGCACCGCCCGCCGCCGCTACACCCGCCGCAAGAACCGCATCTGC |
| | | TACCTGCAGGAGATCTTCAGCAACGAGATGGCCAAGGTGGACGACAGCTT |
| | | CTTCCACCGCCTGGAGGAGAGCTTCCTGGTGGAGGAGGACAAGAAGCACG |
| | | AGCGCCACCCCATCTTCGGCAACATCGTGGACGAGGTGGCCTACCACGAG |
| | | AAGTACCCCACCATCTACCACCTGCGCAAGAAGCTGGTGGACAGCACCGAC |
| | | AAGGCCGACCTGCGCCTGATCTACCTGGCCCTGGCCCACATGATCAAGTTC |
| | | CGCGGCCACTTCCTGATCGAGGGCGACCTGAACCCCGACAACAGCGACGT |
| | | GGACAAGCTGTTCATCCAGCTGGTGCAGACCTACAACCAGCTGTTCGAGGA |
| | | GAACCCCATCAACGCCAGCGGCGTGGACGCCAAGGCCATCCTGAGCGCCC |
| | | GCCTGAGCAAGAGCCGCCGCCTGGAGAACCTGATCGCCCAGCTGCCCGGC |
| | | GAGAAGAAGAACGGCCTGTTCGGCAACCTGATCGCCCTGAGCCTGGGCCT |
| | | GACCCCCAACTTCAAGAGCAACTTCGACCTGGCCGAGGACGCCAAGCTGC |
| | | AGCTGAGCAAGGACACCTACGACGACGACCTGGACAACCTGCTGGCCCAG |
| | | ATCGGCGACCAGTACGCCGACCTGTTCCTGGCCGCCAAGAACCTGAGCGA |
| | | CGCCATCCTGCTGAGCGACATCCTGCGCGTGAACACCGAGATCACCAAGGC |
| | | CCCCCTGAGCGCCAGCATGATCAAGCGCTACGACGAGCACCACCAGGACC |
| | | TGACCCTGCTGAAGGCCCTGGTGCGCCAGCAGCTGCCCGAGAAGTACAAG |
| | | GAGATCTTCTTCGACCAGAGCAAGAACGGCTACGCCGGCTACATCGACGG |
| | | CGGCGCCAGCCAGGAGGAGTTCTACAAGTTCATCAAGCCCATCCTGGAGA |
| | | AGATGGACGGCACCGAGGAGCTGCTGGTGAAGCTGAACCGCGAGGACCT |
| | | GCTGCGCAAGCAGCGCACCTTCGACAACGGCAGCATCCCCCACCAGATCCA |
| | | CCTGGGCGAGCTGCACGCCATCCTGCGCCGCCAGGAGGACTTCTACCCCTT |
| | | CCTGAAGGACAACCGCGAGAAGATCGAGAAGATCCTGACCTTCCGCATCC |
| | | CCTACTACGTGGGCCCCCTGGCCCGCGGCAACAGCCGCTTCGCCTGGATGA |
| | | CCCGCAAGAGCGAGGAGACAATCACCCCCTGGAACTTCGAGGAGGTGGTG |
| | | GACAAGGGCGCCAGCGCCCAGAGCTTCATCGAGCGCATGACCAACTTCGA |
| | | CAAGAACCTGCCCAACGAGAAGGTGCTGCCCAAGCACAGCCTGCTGTACG |
| | | AGTACTTCACCGTGTACAACGAGCTGACCAAGGTGAAGTACGTGACCGAG |
| | | GGCATGCGCAAGCCCGCCTTCCTGAGCGGCGAGCAGAAGAAGGCCATCGT |
| | | GGACCTGCTGTTCAAGACCAACCGCAAGGTGACCGTGAAGCAGCTGAAGG |
| | | AGGACTACTTCAAGAAGATCGAGTGCTTCGACAGCGTGGAGATCAGCGGC |
| | | GTGGAGGACCGCTTCAACGCCAGCCTGGGCACCTACCACGACCTGCTGAA |
| | | GATCATCAAGGACAAGGACTTCCTGGACAACGAGGAGAACGAGGACATCC |
| | | TGGAGGACATCGTGCTGACCCTGACCCTGTTCGAGGACCGCGAGATGATC |
| | | GAGGAGCGCCTGAAGACCTACGCCCACCTGTTCGACGACAAGGTGATGAA |
| | | GCAGCTGAAGCGCCGCCGCTACACCGGCTGGGGCCGCCTGAGCCGCAAGC |
| | | TGATCAACGGCATCCGCGACAAGCAGAGCGGCAAGACCATCCTGGACTTC |
| | | CTCAAGAGCGACGGCTTCGCCAACCGCAACTTCATGCAGCTGATCCACGAC |
| | | GACAGCCTGACCTTCAAGGAGGACATCCAGAAGGCCCAGGTGAGCGGCCA |
| | | GGGCGACAGCCTGCACGAGCACATCGCCAACCTGGCCGGCAGCCCCGCCA |
| | | TCAAGAAGGGCATCCTGCAGACCGTGAAGGTGGTGGACGAGCTGGTGAA |
| | | GGTGATGGGCCGCCACAAGCCCGAGAACATCGTGATCGAGATGGCCCGCG |
| | | AGAACCAGACCACCCAGAAGGGCCAGAAGAACAGCCGCGAGCGCATGAA |
| | | GCGCATCGAGGAGGGCATCAAGGAGCTGGGCAGCCAGATCCTGAAGGAG |
| | | CACCCCGTGGAGAACACCCAGCTGCAGAACGAGAAGCTGTACCTGTACTA |
| | | CCTGCAGAACGGCCGCGACATGTACGTGGACCAGGAGCTGGACATCAACC |
| | | GCCTGAGCGACTACGACGTGGACCACATCGTGCCCCAGAGCTTCCTGAAG |
| | | GACGACAGCATCGACAACAAGGTGCTGACCCGCAGCGACAAGAACCGCG |
| | | GCAAGAGCGACAACGTGCCCAGCGAGGAGGTGGTGAAGAAGATGAAGAA |
| | | CTACTGGCGCCAGCTGCTGAACGCCAAGCTGATCACCCAGCGCAAGTTCGA |
| | | CAACCTGACCAAGGCCGAGCGCGGCGGCCTGAGCGAGCTGGACAAGGCC |
| | | GGCTTCATCAAGCGCCAGCTGGTGGAGACACGCCAGATCACCAAGCACGT |
| | | GGCCCAGATCCTGGACAGCCGCATGAACACCAAGTACGACGAGAACGACA |
| | | AGCTGATCCGCGAGGTGAAGGTGATCACCCTCAAGAGCAAGCTGGTGAGC |
| | | GACTTCCGCAAGGACTTCCAGTTCTACAAGGTGCGCGAGATCAACAACTAC |
| | | CACCACGCCCACGACGCCTACCTGAACGCCGTGGTGGGCACCGCCCTGATC |
| | | AAGAAGTACCCCAAGCTGGAGAGCGAGTTCGTGTACGGCGACTACAAGGT |
| | | GTACGACGTGCGCAAGATGATCGCCAAGAGCGAGCAGGAGATCGGCAAG |
| | | GCCACCGCCAAGTACTTCTTCTACAGCAACATCATGAACTTCTTCAAGACCG |
| | | AGATCACCCTGGCCAACGGCGAGATCCGCAAGCGCCCCCTGATCGAGACA |
| | | AACGGCGAGACAGGCGAGATCGTGTGGGACAAGGGCCGCGACTTCGCCA |
| | | CCGTGCGCAAGGTGCTGAGCATGCCCCAGGTGAACATCGTGAAGAAGACC |
| | | GAGGTGCAGACCGGCGGCTTCAGCAAGGAGAGCATCCTGCCCAAGCGCAA |
| | | CAGCGACAAGCTGATCGCCCGCAAGAAGGACTGGGACCCCAAGAAGTACG |
| | | GCGGCTTCGACAGCCCCACCGTGGCCTACAGCGTGCTGGTGGTGGCCAAG |
| | | GTGGAGAAGGGCAAGAGCAAGAAGCTCAAGAGCGTGAAGGAGCTGCTGG |
| | | GCATCACCATCATGGAGCGCAGCAGCTTCGAGAAGAACCCCATCGACTTCC |
| | | TGGAGGCCAAGGGCTACAAGGAGGTGAAGAAGGACCTGATCATCAAGCT |
| | | GCCCAAGTACAGCCTGTTCGAGCTGGAGAACGGCCGCAAGCGCATGCTGG |
| | | CCAGCGCCGGCGAGCTGCAGAAGGGCAACGAGCTGGCCCTGCCCAGCAA |
| | | GTACGTGAACTTCCTGTACCTGGCCAGCCACTACGAGAAGCTGAAGGGCA |
| | | GCCCCGAGGACAACGAGCAGAAGCAGCTGTTCGTGGAGCAGCACAAGCA |
| | | CTACCTGGACGAGATCATCGAGCAGATCAGCGAGTTCAGCAAGCGCGTGA |
| | | TCCTGGCCGACGCCAACCTGGACAAGGTGCTGAGCGCCTACAACAAGCAC |
| | | CGCGACAAGCCCATCCGCGAGCAGGCCGAGAACATCATCCACCTGTTCACC |
| | | CTGACCAACCTGGGCGCCCCCGCCGCCTTCAAGTACTTCGACACCACCATC |
| | | GACCGCAAGCGCTACACCAGCACCAAGGAGGTGCTGGACGCCACCCTGAT |
| | | CCACCAGAGCATCACCGGCCTGTACGAGACACGCATCGACCTGAGCCAGCT |
| | | GGGCGGCGACGAGGGCGCCGACAAGCGCACCGCCGACGGCAGCGAGTTC |
| | | GAGAGCCCCAAGAAGAAGCGCAAGGTGTAAAGTGTCCAGACCATTGTCTT |
| | | CCAACCCCAGCTGGCCTCTAGAACACCCACTGGCCAGTCCTAGAGCTCCTG |
| | | TCCCTACCCACTCTTTGCTACAATAAATGCTGAATGAATCCATCTAGA |
| mRNA | 5′ UTR | AGGACΨCCΨCCCCAΨCCΨCΨCCCΨCΨGΨCCCΨCΨGΨCCCΨCΨGACCC |
| sequence | | ΨGCACΨGΨCCCAGCACC |
| | CDS | AΨGGACAAGAAGΨACAGCAΨCGGCCΨGGCCAΨCGGCACCAACAGCGΨ |
| | | GGGCΨGGGCCGΨGAΨCACCGACGAGΨACAAGGΨGCCCAGCAAGAAGΨ |
| | | ΨCAAGGΨGCΨGGGCAACACCGACCGCCACAGCAΨCAAGAAGAACCΨGA |
| | | ΨCGGCGCCCΨGCΨGΨΨCGACAGCGGCGAGACAGCCGAGGCCACCCGCC |
| | | ΨGAAGCGCACCGCCCGCCGCCGCΨACACCCGCCGCAAGAACCGCAΨCΨG |
| | | CΨACCΨGCAGGAGAΨCΨΨCAGCAACGAGAΨGGCCAAGGΨGGACGACA |
| | | GCΨΨCΨΨCCACCGCCΨGGAGGAGAGCΨΨCCΨGGΨGGAGGAGGACAA |
| | | GAAGCACGAGCGCCACCCCAΨCΨΨCGGCAACAΨCGΨGGACGAGGΨGG |
| | | CCΨACCACGAGAAGΨACCCCACCAΨCΨACCACCΨGCGCAAGAAGCΨGG |
| | | ΨGGACAGCACCGACAAGGCCGACCΨGCGCCΨGAΨCΨACCΨGGCCCΨG |
| | | GCCCACAGAΨCAAGΨΨCCGCGGCCACΨΨCCΨGAΨCGAGGGCGACCΨ |
| | | GAACCCCGACAACAGCGACGΨGGACAAGCΨGΨΨCAΨCCAGCΨGGΨGC |
| | | AGACCΨACAACCAGCΨGΨΨCGAGGAGAACCCCAΨCAACGCCAGCGGCG |
| | | ΨGGACGCCAAGGCCAΨCCΨGAGCGCCCGCCΨGAGCAAGAGCCGCCGCC |
| | | ΨGGAGAACCΨGAΨCGCCCAGCΨGCCCGGCGAGAAGAAGAACGGCCΨG |
| | | ΨΨCGGCAACCΨGAΨCGCCCΨGAGCCΨGGGCCΨGACCCCCAACΨΨCAA |
| | | GAGCAACΨΨCGACCΨGGCCGAGGACGCCAAGCΨGCAGCΨGAGCAAGG |
| | | ACACCΨACGACGACGACCΨGGACAACCΨGCΨGGCCCAGAΨCGGCGACC |
| | | AGΨACGCCGACCΨGΨΨCCΨGGCCGCCAAGAACCΨGAGCGACGCCAΨC |
| | | CΨGCΨGAGCGACAΨCCΨGCGCGΨGAACACCGAGAΨCACCAAGGCCCCC |
| | | CΨGAGCGCCAGCAΨGAΨCAAGCGCΨACGACGAGCACCACCAGGACCΨG |
| | | ACCCΨGCΨGAAGGCCCΨGGΨGCGCCAGCAGCΨGCCCGAGAAGΨACAA |
| | | GGAGAΨCΨΨCΨΨCGACCAGAGCAAGAACGGCΨACGCCGGCΨACAΨCG |
| | | ACGGCGGCGCCAGCCAGGAGGAGΨΨCΨACAAGΨΨCAΨCAAGCCCAΨC |
| | | CΨGGAGAAGAΨGGACGGCACCGAGGAGCΨGCΨGGΨGAAGCΨGAACC |
| | | GCGAGGACCΨGCΨGCGCAAGCAGCGCACCΨΨCGACAACGGCAGCAΨCC |
| | | CCCACCAGAΨCCACCΨGGGCGAGCΨGCACGCCAΨCCΨGCGCCGCCAGG |
| | | AGGACΨΨCΨACCCCΨΨCCΨGAAGGACAACCGCGAGAAGAΨCGAGAAG |
| | | AΨCCΨGACCΨΨCCGCAΨCCCCΨACΨACGΨGGGCCCCCΨGGCCCGCGG |
| | | CAACAGCCGCΨΨCGCCΨGGAGACCCGCAAGAGCGAGGAGACAAΨCAC |
| | | CCCCΨGGAACΨΨCGAGGAGGΨGGΨGGACAAGGGCGCCAGCGCCCAGA |
| | | GCΨΨCAΨCGAGCGCAΨGACCAACΨΨCGACAAGAACCΨGCCCAACGAG |
| | | AAGGΨGCΨGCCCAAGCACAGCCΨGCΨGΨACGAGΨACΨΨCACCGΨGΨ |
| | | ACAACGAGCΨGACCAAGGΨGAAGΨACGΨGACCGAGGGCAΨGCGCAAG |
| | | CCCGCCΨΨCCΨGAGCGGCGAGCAGAAGAAGGCCAΨCGΨGGACCΨGCΨ |
| | | GΨΨCAAGACCAACCGCAAGGΨGACCGΨGAAGCAGCΨGAAGGAGGACΨ |
| | | ACΨΨCAAGAAGAΨCGAGΨGCΨΨCGACAGCGΨGGAGAΨCAGCGGCGΨ |
| | | GGAGGACCGCΨΨCAACGCCAGCCΨGGGCACCΨACCACGACCΨGCΨGAA |
| | | GAΨCAΨCAAGGACAAGGACΨΨCCΨGGACAACGAGGAGAACGAGGACA |
| | | ΨCCΨGGAGGACAΨCGΨGCVGACCCΨGACCCΨGΨΨCGAGGACCGCGA |
| | | GAΨGAΨCGAGGAGCGCCΨGAAGACCΨACGCCCACCΨGΨΨCGACGACA |
| | | AGGΨGAΨGAAGCAGCΨGAAGCGCCGCCGCΨACACCGGCΨGGGGCCGC |
| | | CΨGAGCCGCAAGCΨGAΨCAACGGCAΨCCGCGACAAGCAGAGCGGCAAG |
| | | ACCAΨCCΨGGACΨΨCCΨCAAGAGCGACGGCΨΨCGCCAACCGCAACΨΨ |
| | | CAΨGCAGCΨGAΨCCACGACGACAGCCΨGACCΨΨCAAGGAGGACAΨCC |
| | | AGAAGGCCCAGGΨGAGCGGCCAGGGCGACAGCCΨGCACGAGCACAΨCG |
| | | CCAACCΨGGCCGGCAGCCCCGCCAΨCAAGAAGGGCAΨCCΨGCAGACCG |
| | | ΨGAAGGΨGGΨGGACGAGCΨGGΨGAAGGΨGAΨGGGCCGCCACAAGCC |
| | | CGAGAACAΨCGΨGAΨCGAGAΨGGCCCGCGAGAACCAGACCACCCAGAA |
| | | GGGCCAGAAGAACAGCCGCGAGCGCAΨGAAGCGCAΨCGAGGAGGGCA |
| | | ΨCAAGGAGCΨGGGCAGCCAGAΨCCΨGAAGGAGCACCCCGΨGGAGAAC |
| | | ACCCAGCΨGCAGAACGAGAAGCΨGΨACCΨGΨACΨACCΨGCAGAACGG |
| | | CCGCGACAΨGΨACGΨGGACCAGGAGCΨGGACAΨCAACCGCCΨGAGCG |
| | | ACΨACGACGΨGGACCACAΨCGΨGCCCCAGAGCΨΨCCΨGAAGGACGAC |
| | | AGCAΨCGACAACAAGGΨGCΨGACCCGCAGCGACAAGAACCGCGGCAAG |
| | | AGCGACAACGΨGCCCAGCGAGGAGGΨGGΨGAAGAAGAΨGAAGAACΨA |
| | | CΨGGCGCCAGCΨGCVGAACGCCAAGCVGAΨCACCCAGCGCAAGΨΨCG |
| | | ACAACCΨGACCAAGGCCGAGCGCGGCGGCCΨGAGCGAGCΨGGACAAGG |
| | | CCGGCΨΨCAΨCAAGCGCCAGCΨGGΨGGAGACACGCCAGAΨCACCAAG |
| | | CACGΨGGCCCAGAΨCCΨGGACAGCCGCAΨGAACACCAAGΨACGACGAG |
| | | AACGACAAGCΨGAΨCCGCGAGGΨGAAGGΨGAΨCACCCΨCAAGAGCAA |
| | | GCΨGGΨGAGCGACΨΨCCGCAAGGACΨΨCCAGΨΨCΨACAAGGΨGCGC |
| | | GAGAΨCAACAACΨACCACCACGCCCACGACGCCΨACCΨGAACGCCGΨG |
| | | GΨGGGCACCGCCCΨGAΨCAAGAAGΨACCCCAAGCΨGGAGAGCGAGΨΨ |
| | | CGΨGΨACGGCGACΨACAAGGΨGΨACGACGΨGCGCAAGAΨGAΨCGCC |
| | | AAGAGCGAGCAGGAGAΨCGGCAAGGCCACCGCCAAGΨACΨΨCΨΨCΨA |
| | | CAGCAACAΨCAΨGAACΨΨCΨΨCAAGACCGAGAΨCACCCΨGGCCAACG |
| | | GCGAGAΨCCGCAAGCGCCCCCΨGAΨCGAGACAAACGGCGAGACAGGCG |
| | | AGAΨCGΨGΨGGGACAAGGGCCGCGACΨΨCGCCACCGΨGCGCAAGGΨ |
| | | GCΨGAGCAΨGCCCCAGGΨGAACAΨCGΨGAAGAAGACCGAGGΨGCAGA |
| | | CCGGCGGCΨΨCAGCAAGGAGAGCAΨCCΨGCCCAAGCGCAACAGCGACA |
| | | AGCΨGAΨCGCCCGCAAGAAGGACΨGGGACCCCAAGAAGΨACGGCGGC |
| | | ΨΨCGACAGCCCCACCGΨGGCCΨACAGCGΨGCΨGGΨGGΨGGCCAAGG |
| | | ΨGGAGAAGGGCAAGAGCAAGAAGCΨCAAGAGCGΨGAAGGAGCΨGCΨ |
| | | GGGCAΨCACCAΨCAΨGGAGCGCAGCAGCΨΨCGAGAAGAACCCCAΨCG |
| | | ACΨΨCCΨGGAGGCCAAGGGCΨACAAGGAGGΨGAAGAAGGACCΨGAΨ |
| | | CAΨCAAGCΨGCCCAAGΨACAGCCΨGΨΨCGAGCΨGGAGAACGGCCGCA |
| | | AGCGCAΨGCΨGGCCAGCGCCGGCGAGCΨGCAGAAGGGCAACGAGCΨG |
| | | GCCCΨGCCCAGCAAGΨACGΨGAACΨΨCCΨGΨACCΨGGCCAGCCACΨA |
| | | CGAGAAGCΨGAAGGGCAGCCCCGAGGACAACGAGCAGAAGCAGCΨGΨ |
| | | ΨCGΨGGAGCAGCACAAGCACΨACCΨGGACGAGAΨCAΨCGAGCAGAΨC |
| | | AGCGAGΨΨCAGCAAGCGCGΨGAΨCCΨGGCCGACGCCAACCΨGGACAA |
| | | GGΨGCΨGAGCGCCΨACAACAAGCACCGCGACAAGCCCAΨCCGCGAGCA |
| | | GGCCGAGAACAΨCAΨCCACCΨGΨΨCACCCΨGACCAACCΨGGGCGCCCC |
| | | CGCCGCCΨΨCAAGΨACΨΨCGACACCACCAΨCGACCGCAAGCGCΨACAC |
| | | CAGCACCAAGGAGGΨGCΨGGACGCCACCCΨGAΨCCACCAGAGCAΨCAC |
| | | CGGCCΨGΨACGAGACACGCAΨCGACCΨGAGCCAGCΨGGGCGGCGACG |
| | | AGGGCGCCGACAAGCGCACCGCCGACGGCAGCGAGΨΨCGAGAGCCCCA |
| | | AGAAGAAGCGCAAGGΨGΨAA |
| | 3′ UTR | AGΨGΨCCAGACCAΨΨGΨCΨΨCCAACCCCAGCΨGGCCΨCΨAGAACACC |
| | | CACΨGGCCAGΨCCΨAGAGCΨCCΨGΨCCCΨACCCACΨCΨΨΨGCΨACA |
| | | AΨAAAΨGCΨGAAΨGAAΨCCAΨCΨAGA |
| | Poly(A) | Between 80-150 nucleotides length. |

SpCas9-H840A

| | | |
|---|---|---|
| Full | Protein | MDKKYSIGLDIGTNSVGWAVITDEYKVPSKKFKVLGNTDRHSIKKNLIGALLFDS |
| Sequence | | GETAEATRLKRTARRRYTRRKNRICYLQEIFSNEMAKVDDSFFHRLEESFLVEED |
| | | KKHERHPIFGNIVDEVAYHEKYPTIYHLRKKLVDSTDKADLRLIYLALAHMIKFR |
| | | GHFLIEGDLNPDNSDVDKLFIQLVQTYNQLFEENPINASGVDAKAILSARLSKSR |
| | | RLENLIAQLPGEKKNGLFGNLIALSLGLTPNFKSNFDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTFDNGSIPHQIHLGELHAILRRQEDFYPFLKDNREKIE |
| | | KILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLKRRRYTGW |
| | | GRLSRKLINGIRDKQSGKTILDFLKSDGFANRNFMQLIHDDSLTFKEDIQKAQV |
| | | SGQGDSLHEHIANLAGSPAIKKGILQTVKVVDELVKVMGRHKPENIVIEMARE |
| | | NQTTQKGQKNSRERMKRIEEGIKELGSQILKEHPVENTQLQNEKLYLYYLQNG |
| | | RDMYVDQELDINRLSDYDVDAIVPQSFLKDDSIDNKVLTRSDKNRGKSDNVPS |
| | | EEVVKKMKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYKVREINN |
| | | YHHAHDAYLNAVVGTALIKKYPKLESEFVYGDYKVYDVRKMIAKSEQEIGKATA |
| | | KYFFYSNIMNFFKTEITLANGEIRKRPLIETNGETGEIVWDKGRDFATVRKVLSM |
| | | PQVNIVKKTEVQTGGFSKESILPKRNSDKLIARKKDWDPKKYGGFDSPTVAYSV |
| | | LVVAKVEKGKSKKLKSVKELLGITIMERSSFEKNPIDFLEAKGYKEVKKDLIIKLPK |
| | | YSLFELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAENII |
| | | HLFTLTNLGAPAAFKYFDTTIDRKRYTSTKEVLDATLIHQSITGLYETRIDLSQLG |
| | | GDEGADKRTADGSEFESPKKKRKV* |
| | DNA | AGGACTCCTCCCCATCCTCTCCCTCTGTCCCTCTGTCCCTCTGACCCTGCACT |
| | | GTCCCAGCACCATGGACAAGAAGTACAGCATCGGCCTGGACATCGGCACC |
| | | AACAGCGTGGGCTGGGCCGTGATCACCGACGAGTACAAGGTGCCCAGCAA |
| | | GAAGTTCAAGGTGCTGGGCAACACCGACCGCCACAGCATCAAGAAGAACC |
| | | TGATCGGCGCCCTGCTGTTCGACAGCGGCGAGACAGCCGAGGCCACCCGC |
| | | CTGAAGCGCACCGCCCGCCGCCGCTACACCCGCCGCAAGAACCGCATCTGC |
| | | TACCTGCAGGAGATCTTCAGCAACGAGATGGCCAAGGTGGACGACAGCTT |
| | | CTTCCACCGCCTGGAGGAGAGCTTCCTGGTGGAGGAGGACAAGAAGCACG |
| | | AGCGCCACCCCATCTTCGGCAACATCGTGGACGAGGTGGCCTACCACGAG |
| | | AAGTACCCCACCATCTACCACCTGCGCAAGAAGCTGGTGGACAGCACCGAC |
| | | AAGGCCGACCTGCGCCTGATCTACCTGGCCCTGGCCCACATGATCAAGTTC |
| | | CGCGGCCACTTCCTGATCGAGGGCGACCTGAACCCCGACAACAGCGACGT |
| | | GGACAAGCTGTTCATCCAGCTGGTGCAGACCTACAACCAGCTGTTCGAGGA |
| | | GAACCCCATCAACGCCAGCGGCGTGGACGCCAAGGCCATCCTGAGCGCCC |
| | | GCCTGAGCAAGAGCCGCCGCCTGGAGAACCTGATCGCCCAGCTGCCCGGC |
| | | GAGAAGAAGAACGGCCTGTTCGGCAACCTGATCGCCCTGAGCCTGGGCCT |
| | | GACCCCCAACTTCAAGAGCAACTTCGACCTGGCCGAGGACGCCAAGCTGC |
| | | AGCTGAGCAAGGACACCTACGACGACGACCTGGACAACCTGCTGGCCCAG |
| | | ATCGGCGACCAGTACGCCGACCTGTTCCTGGCCGCCAAGAACCTGAGCGA |
| | | CGCCATCCTGCTGAGCGACATCCTGCGCGTGAACACCGAGATCACCAAGGC |
| | | CCCCCTGAGCGCCAGCATGATCAAGCGCTACGACGAGCACCACCAGGACC |
| | | TGACCCTGCTGAAGGCCCTGGTGCGCCAGCAGCTGCCCGAGAAGTACAAG |
| | | GAGATCTTCTTCGACCAGAGCAAGAACGGCTACGCCGGCTACATCGACGG |
| | | CGGCGCCAGCCAGGAGGAGTTCTACAAGTTCATCAAGCCCATCCTGGAGA |
| | | AGATGGACGGCACCGAGGAGCTGCTGGTGAAGCTGAACCGCGAGGACCT |
| | | GCTGCGCAAGCAGCGCACCTTCGACAACGGCAGCATCCCCCACCAGATCCA |
| | | CCTGGGCGAGCTGCACGCCATCCTGCGCCGCCAGGAGGACTTCTACCCCTT |
| | | CCTGAAGGACAACCGCGAGAAGATCGAGAAGATCCTGACCTTCCGCATCC |
| | | CCTACTACGTGGGCCCCCTGGCCCGCGGCAACAGCCGCTTCGCCTGGATGA |
| | | CCCGCAAGAGCGAGGAGACAATCACCCCCTGGAACTTCGAGGAGGTGGTG |
| | | GACAAGGGCGCCAGCGCCCAGAGCTTCATCGAGCGCATGACCAACTTCGA |
| | | CAAGAACCTGCCCAACGAGAAGGTGCTGCCCAAGCACAGCCTGCTGTACG |
| | | AGTACTTCACCGTGTACAACGAGCTGACCAAGGTGAAGTACGTGACCGAG |
| | | GGCATGCGCAAGCCCGCCTTCCTGAGCGGCGAGCAGAAGAAGGCCATCGT |
| | | GGACCTGCTGTTCAAGACCAACCGCAAGGTGACCGTGAAGCAGCTGAAGG |
| | | AGGACTACTTCAAGAAGATCGAGTGCTTCGACAGCGTGGAGATCAGCGGC |
| | | GTGGAGGACCGCTTCAACGCCAGCCTGGGCACCTACCACGACCTGCTGAA |
| | | GATCATCAAGGACAAGGACTTCCTGGACAACGAGGAGAACGAGGACATCC |
| | | TGGAGGACATCGTGCTGACCCTGACCCTGTTCGAGGACCGCGAGATGATC |
| | | GAGGAGCGCCTGAAGACCTACGCCCACCTGTTCGACGACAAGGTGATGAA |
| | | GCAGCTGAAGCGCCGCCGCTACACCGGCTGGGGCCGCCTGAGCCGCAAGC |
| | | TGATCAACGGCATCCGCGACAAGCAGAGCGGCAAGACCATCCTGGACTTC |
| | | CTCAAGAGCGACGGCTTCGCCAACCGCAACTTCATGCAGCTGATCCACGAC |
| | | GACAGCCTGACCTTCAAGGAGGACATCCAGAAGGCCCAGGTGAGCGGCCA |
| | | GGGCGACAGCCTGCACGAGCACATCGCCAACCTGGCCGGCAGCCCCGCCA |
| | | TCAAGAAGGGCATCCTGCAGACCGTGAAGGTGGTGGACGAGCTGGTGAA |
| | | GGTGATGGGCCGCCACAAGCCCGAGAACATCGTGATCGAGATGGCCCGCG |
| | | AGAACCAGACCACCCAGAAGGGCCAGAAGAACAGCCGCGAGCGCATGAA |
| | | GCGCATCGAGGAGGGCATCAAGGAGCTGGGCAGCCAGATCCTGAAGGAG |
| | | CACCCCGTGGAGAACACCCAGCTGCAGAACGAGAAGCTGTACCTGTACTA |
| | | CCTGCAGAACGGCCGCGACATGTACGTGGACCAGGAGCTGGACATCAACC |
| | | GCCTGAGCGACTACGACGTGGACGCCATCGTGCCCCAGAGCTTCCTGAAG |
| | | GACGACAGCATCGACAACAAGGTGCTGACCCGCAGCGACAAGAACCGCG |
| | | GCAAGAGCGACAACGTGCCCAGCGAGGAGGTGGTGAAGAAGATGAAGAA |
| | | CTACTGGCGCCAGCTGCTGAACGCCAAGCTGATCACCCAGCGCAAGTTCGA |
| | | CAACCTGACCAAGGCCGAGCGCGGCGGCCTGAGCGAGCTGGACAAGGCC |
| | | GGCTTCATCAAGCGCCAGCTGGTGGAGACACGCCAGATCACCAAGCACGT |
| | | GGCCCAGATCCTGGACAGCCGCATGAACACCAAGTACGACGAGAACGACA |
| | | AGCTGATCCGCGAGGTGAAGGTGATCACCCTCAAGAGCAAGCTGGTGAGC |
| | | GACTTCCGCAAGGACTTCCAGTTCTACAAGGTGCGCGAGATCAACAACTAC |
| | | CACCACGCCCACGACGCCTACCTGAACGCCGTGGTGGGCACCGCCCTGATC |
| | | AAGAAGTACCCCAAGCTGGAGAGCGAGTTCGTGTACGGCGACTACAAGGT |
| | | GTACGACGTGCGCAAGATGATCGCCAAGAGCGAGCAGGAGATCGGCAAG |
| | | GCCACCGCCAAGTACTTCTTCTACAGCAACATCATGAACTTCTTCAAGACCG |
| | | AGATCACCCTGGCCAACGGCGAGATCCGCAAGCGCCCCCTGATCGAGACA |
| | | AACGGCGAGACAGGCGAGATCGTGTGGGACAAGGGCCGCGACTTCGCCA |
| | | CCGTGCGCAAGGTGCTGAGCATGCCCCAGGTGAACATCGTGAAGAAGACC |
| | | GAGGTGCAGACCGGCGGCTTCAGCAAGGAGAGCATCCTGCCCAAGCGCAA |
| | | CAGCGACAAGCTGATCGCCCGCAAGAAGGACTGGGACCCCAAGAAGTACG |
| | | GCGGCTTCGACAGCCCCACCGTGGCCTACAGCGTGCTGGTGGTGGCCAAG |
| | | GTGGAGAAGGGCAAGAGCAAGAAGCTCAAGAGCGTGAAGGAGCTGCTGG |
| | | GCATCACCATCATGGAGCGCAGCAGCTTCGAGAAGAACCCCATCGACTTCC |
| | | TGGAGGCCAAGGGCTACAAGGAGGTGAAGAAGGACCTGATCATCAAGCT |
| | | GCCCAAGTACAGCCTGTTCGAGCTGGAGAACGGCCGCAAGCGCATGCTGG |
| | | CCAGCGCCGGCGAGCTGCAGAAGGGCAACGAGCTGGCCCTGCCCAGCAA |
| | | GTACGTGAACTTCCTGTACCTGGCCAGCCACTACGAGAAGCTGAAGGGCA |
| | | GCCCCGAGGACAACGAGCAGAAGCAGCTGTTCGTGGAGCAGCACAAGCA |
| | | CTACCTGGACGAGATCATCGAGCAGATCAGCGAGTTCAGCAAGCGCGTGA |
| | | TCCTGGCCGACGCCAACCTGGACAAGGTGCTGAGCGCCTACAACAAGCAC |
| | | CGCGACAAGCCCATCCGCGAGCAGGCCGAGAACATCATCCACCTGTTCACC |
| | | CTGACCAACCTGGGCGCCCCCGCCGCCTTCAAGTACTTCGACACCACCATC |
| | | GACCGCAAGCGCTACACCAGCACCAAGGAGGTGCTGGACGCCACCCTGAT |
| | | CCACCAGAGCATCACCGGCCTGTACGAGACACGCATCGACCTGAGCCAGCT |
| | | GGGCGGCGACGAGGGCGCCGACAAGCGCACCGCCGACGGCAGCGAGTTC |
| | | GAGAGCCCCAAGAAGAAGCGCAAGGTGTAAAGTGTCCAGACCATTGTCTT |
| | | CCAACCCCAGCTGGCCTCTAGAACACCCACTGGCCAGTCCTAGAGCTCCTG |
| | | TCCCTACCCACTCTTTGCTACAATAAATGCTGAATGAATCCATCTAGA |
| mRNA | 5′ UTR | AGGACΨCCΨCCCCAΨCCΨCΨCCCΨCΨGΨCCCΨCΨGΨCCCΨCΨGACCC |
| sequence | | ΨGCACΨGΨCCCAGCACC |
| | CDS | AΨGGACAAGAAGΨACAGCAΨCGGCCΨGGACAΨCGGCACCAACAGCGΨ |
| | | GGGCΨGGGCCGΨGAΨCACCGACGAGΨACAAGGΨGCCCAGCAAGAAGΨ |
| | | ΨCAAGGΨGCΨGGGCAACACCGACCGCCACAGCAΨCAAGAAGAACCΨGA |
| | | ΨCGGCGCCCΨGCΨGΨΨCGACAGCGGCGAGACAGCCGAGGCCACCCGCC |
| | | ΨGAAGCGCACCGCCCGCCGCCGCΨACACCCGCCGCAAGAACCGCAΨCΨG |
| | | CΨACCΨGCAGGAGAΨCΨΨCAGCAACGAGAΨGGCCAAGGΨGGACGACA |
| | | GCΨΨCΨΨCCACCGCCΨGGAGGAGAGCΨΨCCΨGGΨGGAGGAGGACAA |
| | | GAAGCACGAGCGCCACCCCAΨCΨΨCGGCAACAΨCGΨGGACGAGGΨGG |
| | | CCΨACCACGAGAAGΨACCCCACCAΨCΨACCACCΨGCGCAAGAAGCΨGG |
| | | ΨGGACAGCACCGACAAGGCCGACCΨGCGCCΨGAΨCΨACCΨGGCCCΨG |
| | | GCCCACAΨGAΨCAAGΨΨCCGCGGCCACΨΨCCΨGAΨCGAGGGCGACCΨ |
| | | GAACCCCGACAACAGCGACGΨGGACAAGCΨGΨΨCAΨCCAGCΨGGΨGC |
| | | AGACCΨACAACCAGCΨGΨΨCGAGGAGAACCCCAΨCAACGCCAGCGGCG |
| | | ΨGGACGCCAAGGCCAΨCCΨGAGCGCCCGCCΨGAGCAAGAGCCGCCGCC |
| | | ΨGGAGAACCΨGAΨCGCCCAGCΨGCCCGGCGAGAAGAAGAACGGCCΨG |
| | | ΨΨCGGCAACCΨGAΨCGCCCΨGAGCCGGGCCΨGACCCCCAACΨΨCAA |
| | | GAGCAACΨΨCGACCΨGGCCGAGGACGCCAAGCΨGCAGCΨGAGCAAGG |
| | | ACACCΨACGACGACGACCΨGGACAACCΨGCΨGGCCCAGAΨCGGCGACC |
| | | AGΨACGCCGACCΨGΨΨCCΨGGCCGCCAAGAACCΨGAGCGACGCCAΨC |
| | | CΨGCΨGAGCGACAΨCCΨGCGCGΨGAACACCGAGAΨCACCAAGGCCCCC |
| | | CΨGAGCGCCAGCAΨGAΨCAAGCGCΨACGACGAGCACCACCAGGACCΨG |
| | | ACCCΨGCΨGAAGGCCCΨGGΨGCGCCAGCAGCΨGCCCGAGAAGΨACAA |
| | | GGAGAΨCΨΨCΨΨCGACCAGAGCAAGAACGGCΨACGCCGGCΨACAΨCG |
| | | ACGGCGGCGCCAGCCAGGAGGAGΨΨCΨACAAGΨΨCAΨCAAGCCCAΨC |
| | | CΨGGAGAAGAΨGGACGGCACCGAGGAGCΨGCΨGGΨGAAGCΨGAACC |
| | | GCGAGGACCΨGCΨGCGCAAGCAGCGCACCΨΨCGACAACGGCAGCACC |
| | | CCCACCAGAΨCCACCΨGGGCGAGCΨGCACGCCAΨCCΨGCGCCGCCAGG |
| | | AGGACΨΨCΨACCCCΨΨCCΨGAAGGACAACCGCGAGAAGAΨCGAGAAG |
| | | AΨCCΨGACCΨΨCCGCAΨCCCCΨACΨACGΨGGGCCCCCΨGGCCCGCGG |
| | | CAACAGCCGCΨΨCGCCΨGGAΨGACCCGCAAGAGCGAGGAGACAAΨCAC |
| | | CCCCΨGGAACΨΨCGAGGAGGΨGGΨGGACAAGGGCGCCAGCGCCCAGA |
| | | GCΨΨCAΨCGAGCGCAΨGACCAACΨΨCGACAAGAACCΨGCCCAACGAG |
| | | AAGGΨGCΨGCCCAAGCACAGCCΨGCΨGΨACGAGΨACΨΨCACCGΨGΨ |
| | | ACAACGAGCΨGACCAAGGΨGAAGΨACGΨGACCGAGGGCAΨGCGCAAG |
| | | CCCGCCΨΨCCΨGAGCGGCGAGCAGAAGAAGGCCAΨCGΨGGACCΨGCΨ |
| | | GΨΨCAAGACCAACCGCAAGGΨGACCGΨGAAGCAGCΨGAAGGAGGACΨ |
| | | ACΨΨCAAGAAGAΨCGAGΨGCΨΨCGACAGCGΨGGAGAΨCAGCGGCGΨ |
| | | GGAGGACCGCΨΨCAACGCCAGCCΨGGGCACCΨACCACGACCΨGCΨGAA |
| | | GAΨCAΨCAAGGACAAGGACΨΨCCΨGGACAACGAGGAGAACGAGGACA |
| | | ΨCCΨGGAGGACAΨCGΨGCΨGACCCΨGACCCΨGΨΨCGAGGACCGCGA |
| | | GAΨGAΨCGAGGAGCGCCΨGAAGACCΨACGCCCACCΨGΨΨCGACGACA |
| | | AGGΨGAΨGAAGCAGCΨGAAGCGCCGCCGCΨACACCGGCΨGGGGCCGC |
| | | CΨGAGCCGCAAGCΨGAΨCAACGGCAΨCCGCGACAAGCAGAGCGGCAAG |
| | | ACCAΨCCΨGGACΨΨCCΨCAAGAGCGACGGCΨΨCGCCAACCGCAACΨΨ |
| | | CAΨGCAGCΨGAΨCCACGACGACAGCCΨGACCΨΨCAAGGAGGACAΨCC |
| | | AGAAGGCCCAGGΨGAGCGGCCAGGGCGACAGCCΨGCACGAGCACAΨCG |
| | | CCAACCΨGGCCGGCAGCCCCGCCAΨCAAGAAGGGCAΨCCΨGCAGACCG |
| | | ΨGAAGGΨGGGGACGAGCΨGGΨGAAGGΨGAΨGGGCCGCCACAAGCC |
| | | CGAGAACAΨCGΨGAΨCGAGAΨGGCCCGCGAGAACCAGACCACCCAGAA |
| | | GGGCCAGAAGAACAGCCGCGAGCGCAΨGAAGCGCAΨCGAGGAGGGCA |
| | | ΨCAAGGAGCΨGGGCAGCCAGAΨCCΨGAAGGAGCACCCCGΨGGAGAAC |
| | | ACCCAGCΨGCAGAACGAGAAGCΨGΨACCΨGΨACΨACCΨGCAGAACGG |
| | | CCGCGACAΨGΨACGΨGGACCAGGAGCΨGGACAΨCAACCGCCΨGAGCG |
| | | ACΨACGACGΨGGACGCCAΨCGΨGCCCCAGAGCΨΨCCΨGAAGGACGAC |
| | | AGCAΨCGACAACAAGGΨGCΨGACCCGCAGCGACAAGAACCGCGGCAAG |
| | | AGCGACAACGΨGCCCAGCGAGGAGGΨGGΨGAAGAAGAΨGAAGAACΨA |
| | | CΨGGCGCCAGCΨGCVGAACGCCAAGCVGAΨCACCCAGCGCAAGΨΨCG |
| | | ACAACCΨGACCAAGGCCGAGCGCGGCGGCCΨGAGCGAGCΨGGACAAGG |
| | | CCGGCΨΨCAΨCAAGCGCCAGCΨGGΨGGAGACACGCCAGAΨCACCAAG |
| | | CACGΨGGCCCAGAΨCCΨGGACAGCCGCAΨGAACACCAAGΨACGACGAG |
| | | AACGACAAGCΨGAΨCCGCGAGGΨGAAGGΨGAΨCACCCΨCAAGAGCAA |
| | | GCΨGGΨGAGCGACΨΨCCGCAAGGACΨΨCCAGΨΨCΨACAAGGΨGCGC |
| | | GAGAΨCAACAACΨACCACCACGCCCACGACGCCΨACCΨGAACGCCGΨG |
| | | GΨGGGCACCGCCCΨGAΨCAAGAAGΨACCCCAAGCΨGGAGAGCGAGΨΨ |
| | | CGΨGΨACGGCGACΨACAAGGΨGΨACGACGΨGCGCAAGAΨGAΨCGCC |
| | | AAGAGCGAGCAGGAGAΨCGGCAAGGCCACCGCCAAGΨACΨΨCΨΨCΨA |
| | | CAGCAACAΨCAΨGAACΨΨCΨΨCAAGACCGAGAΨCACCCΨGGCCAACG |
| | | GCGAGAΨCCGCAAGCGCCCCCΨGAΨCGAGACAAACGGCGAGACAGGCG |
| | | AGAΨCGΨGΨGGGACAAGGGCCGCGACΨΨCGCCACCGΨGCGCAAGGΨ |
| | | GCΨGAGCAΨGCCCCAGGΨGAACAΨCGΨGAAGAAGACCGAGGΨGCAGA |
| | | CCGGCGGCΨΨCAGCAAGGAGAGCAΨCCΨGCCCAAGCGCAACAGCGACA |
| | | AGCΨGAΨCGCCCGCAAGAAGGACΨGGGACCCCAAGAAGΨACGGCGGC |
| | | ΨΨCGACAGCCCCACCGΨGGCCΨACAGCGΨGCΨGGΨGGΨGGCCAAGG |
| | | ΨGGAGAAGGGCAAGAGCAAGAAGCΨCAAGAGCGΨGAAGGAGCΨGCΨ |
| | | GGGCAΨCACCAΨCAΨGGAGCGCAGCAGCΨΨCGAGAAGAACCCCAΨCG |
| | | ACΨΨCCΨGGAGGCCAAGGGCΨACAAGGAGGΨGAAGAAGGACCΨGAΨ |
| | | CAΨCAAGCΨGCCCAAGΨACAGCCΨGΨΨCGAGCΨGGAGAACGGCCGCA |
| | | AGCGCAΨGCΨGGCCAGCGCCGGCGAGCΨGCAGAAGGGCAACGAGCΨG |
| | | GCCCΨGCCCAGCAAGΨACGΨGAACΨΨCCΨGΨACCΨGGCCAGCCACΨA |
| | | CGAGAAGCΨGAAGGGCAGCCCCGAGGACAACGAGCAGAAGCAGCΨGΨ |
| | | ΨCGΨGGAGCAGCACAAGCACΨACCΨGGACGAGAΨCAΨCGAGCAGAΨC |
| | | AGCGAGΨΨCAGCAAGCGCGΨGAΨCCΨGGCCGACGCCAACCΨGGACAA |
| | | GGΨGCΨGAGCGCCΨACAACAAGCACCGCGACAAGCCCAΨCCGCGAGCA |
| | | GGCCGAGAACAΨCAΨCCACCΨGΨΨCACCCΨGACCAACCΨGGGCGCCCC |
| | | CGCCGCCΨΨCAAGΨACΨΨCGACACCACCAΨCGACCGCAAGCGCΨACAC |
| | | CAGCACCAAGGAGGΨGCΨGGACGCCACCCΨGAΨCCACCAGAGCAΨCAC |
| | | CGGCCΨGΨACGAGACACGCAΨCGACCΨGAGCCAGCΨGGGCGGCGACG |
| | | AGGGCGCCGACAAGCGCACCGCCGACGGCAGCGAGΨΨCGAGAGCCCCA |
| | | AGAAGAAGCGCAAGGΨGAA |
| | 3′ UTR | AGΨGΨCCAGACCAΨΨGΨCΨΨCCAACCCCAGCΨGGCCΨCΨAGAACACC |
| | | CACΨGGCCAGΨCCΨAGAGCΨCCΨGΨCCCΨACCCACΨCΨΨΨGCΨACA |
| | | AΨAAAΨGCΨGAAΨGAAΨCCAΨCΨAGA |
| | Poly(A) | Between 80-150 nucleotides length. |

Legend
Ψ N¹-methyl pseudouridine

In some embodiments, an mRNA encoding a nickase has a sequence of an mRNA listed in Table 1. In some embodiments, an mRNA encoding a nickase has a sequence that is at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to an mRNA sequence listed in Table 1.

In some embodiments, an mRNA encoding a nickase has a coding sequence of an mRNA listed in Table 1. In some embodiments, an mRNA encoding a nickase has a coding sequence that is at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to a coding sequence of an mRNA sequence listed in Table 1.

It will be understood that the coding sequence of an mRNA listed in Table 1 may be modified or optimized, the untranslated regions (UTRs), and/or the 5′ and 3′ ends (e.g., within three or within five nucleotides of the end) may be modified or optimized to enhance or alter stability and/or expression of the mRNA. In some embodiments, the mRNA may comprise one or more stabilizing motif, such as a 5′ and/or 3′ end stabilizing motif.

As described herein, dual nickase editing systems are disclosed to effectuate editing in the LPA gene.

### IV. GUIDE NUCLEIC ACIDS AND TARGETED DNA SEQUENCES

In embodiments, a dual nickase editing system comprises more than one guide nucleic acid. In some embodiments, a dual nickase editing system comprises two guide nucleic acids.

A guide nucleic acid sequence directs a gene editor to a target genomic location. The guide nucleic acid may vary depending on the gene editor. The present disclosure refers to guide nucleic acids as guide nucleic acid sequences, guide RNAs, gRNAs and/or guide oligonucleotides. As described herein, guide RNAs may comprise an RNA sequence that may or may not be chemically modified. While gRNAs commonly comprise RNA sequences, it should be understood that portions of a guide RNA may not be ribonucleic acids but may comprise deoxyribonucleic acid or other chemical substitutions, including nucleotide analogs.

Each guide nucleic acid may include a nucleotide sequence that is complementary to a targeted site in chromosomal DNA. The portion of the guide nucleic acid that specifies the targeted site is referred to herein as the “spacer.” The spacer is typically located at the 5′ end of the guide nucleic acid. The strand of the chromosomal DNA that contains a sequence that is complementary to the spacer is referred to herein as the “target strand.” The non-target strand, also referred to as the “displaced strand”, of the chromosomal DNA harbors a sequence that is identical to, or substantially identical to, the spacer, referred to herein as the “protospacer.”

In one or more embodiments, each guide nucleic acid includes a spacer sequence that is identical to or substantially identical to a protospacer sequence of the LPA gene. In embodiments, the gene editing system comprises two guide nucleic acids, each having different spacer sequences, wherein each spacer sequence is identical or substantially identical to a protospacer sequence specified in Table 2 or Table 5. In some embodiments, a guide oligonucleotide comprises a spacer comprising a sequence identical to or substantially identical to nucleotides 6 to 20, 5 to 20, 4 to 20, 3 to 20, 2 to 20, or 1 to 20 of a protospacer sequence specified in Table 2 or Table 5. In some embodiments, a guide oligonucleotide comprises a spacer comprising a sequence identical to or substantially identical to nucleotides 6 to 20, 5 to 20, 4 to 20, 3 to 20, 2 to 20, or 1 to 20 of a protospacer sequence specified in Table 2 or Table 5 with 0, 1, 2, 3, 4, or 5 mismatches. In some embodiments, a guide oligonucleotide comprises a spacer having a sequence that is at least about 75%, at least about 80%, at least about 85%, at least about 90%, or at least about 95% identical to a protospacer sequence specified in Table 2 or Table 5. In some embodiments, a guide oligonucleotide comprises a spacer having a sequence that is identical to a protospacer sequence specified in Table 2 or Table 5.

In embodiments, a gene editing system described herein comprises a first guide oligonucleotide and a second guide oligonucleotide, where the first guide oligonucleotide comprises a spacer sequence identical to or substantially identical to a guide 1 protospacer sequence specified in Table 2 or Table 5, and the second guide oligonucleotide comprises a spacer sequence identical to or substantially identical to a corresponding (in the same row) guide 2 protospacer sequence specified in Table 2 or Table 5. In some embodiments, the first guide oligonucleotide comprises a spacer sequence identical to or substantially identical to nucleotides 6 to 20, 5 to 20, 4 to 20, 3 to 20, 2 to 20, or 1 to 20 of a guide 1 protospacer sequence specified in Table 2 or Table 5, and the second guide oligonucleotide comprises a spacer sequence identical to or substantially identical to nucleotides 6 to 20, 5 to 20, 4 to 20, 3 to 20, 2 to 20, or 1 to 20 of a corresponding (in the same row) guide 2 protospacer sequence specified in Table 2 or Table 5. In some embodiments, the first guide oligonucleotide comprises a spacer sequence identical to or substantially identical to nucleotides 6 to 20, 5 to 20, 4 to 20, 3 to 20, 2 to 20, or 1 to 20 of a guide 1 protospacer sequence specified in Table 2 or Table 5 with 0, 1, 2, 3, 4, or 5 mismatches, and the second guide oligonucleotide comprises a spacer sequence identical to or substantially identical to nucleotides 6 to 20, 5 to 20, 4 to 20, 3 to 20, 2 to 20, or 1 to 20 of a corresponding (in the same row) guide 2 protospacer sequence specified in Table 2 or Table 5 with 0, 1, 2, 3, 4, or 5 mismatches. In some embodiments, the first guide oligonucleotide comprises a spacer having a sequence that is at least about 75%, at least about 80%, at least about 85%, at least about 90%, or at least about 95 identical to a guide 1 protospacer sequence specified in Table 2 or Table 5, and the second guide oligonucleotide comprises a spacer having a sequence that is at least about 75%, at least about 80%, at least about 85%, at least about 90%, or at least about 95% identical to a corresponding (in the same row) guide 2 protospacer sequence specified in Table 2 or Table 5. In some embodiments, the first guide oligonucleotide comprises a spacer sequence identical to a guide 1 protospacer sequence specified in Table 2 or Table 5, and the second guide oligonucleotide comprises a spacer sequence identical to a corresponding (in the same row) guide 2 protospacer sequence specified in Table 2 or Table 5.

TABLE 2
Examples of protospacers to which spacers of guide oligonucleotides
may correspond

| | MIT | | MIT |
|---|---|---|---|
| Guide 1 Protospacer + PAM | Score | Guide 2 Protospacer + PAM | Score |
| GGAGCCAGAATAACATTCGGAGG | 87 | CTAGAGGCTTTTTTTGAACAAGG | 57 |
| CCATCACTGGACATTGCGTCAGG | 86 | AACTCTCCTCACAACTCCCACGG | 60 |
| GAGCCAGAATAACATTCGGAGGG | 83 | CTAGAGGCTTTTTTTGAACAAGG | 57 |
| AATAACATTCGGAGGGACGAAGG | 82 | TATTCTGGCTCCAAGCCTAGAGG | 70 |
| TGACACCACATTGGCATCGGAGG | 82 | ACATGTTCTTCCTGTGATAGTGG | 41 |
| CTACTTAGAAGCTGAAACGTGGG | 81 | CCACGTTTCAGCTTCTAAGTAGG | 60 |
| ACCCCGGGGCTTTGCTCCGTTGG | 80 | CAGGATTGCTACCATGGTGATGG | 19 |
| AACTCAAGGTTGTACCCATTTGG | 76 | CAACAGAATATTATCCAAATGGG | 52 |
| GGTAATAGTTGGAGGCGCGACGG | 75 | TACCCCGATTCCAAGCCTAGAGG | 12 |
| GGGATCCTCCGATGCCAATGTGG | 74 | TGACACCACATTGGCATCGGAGG | 82 |
| CTGCATCTGAGCATCGTGTCAGG | 72 | CGTCCCTCCGAATGTTATTCTGG | 84 |
| CTTGGAACTGGGACCACCGTGGG | 72 | ACAGAGCTTCCTTCTGAAGAAGG | 46 |
| CCTACTTAGAAGCTGAAACGTGG | 71 | CCACGTTTCAGCTTCTAAGTAGG | 60 |
| CATAGATGACCAAGATTGACAGG | 70 | TGATACCACACTGGCATCAGAGG | 37 |
| TGACACCACATCAACATAATAGG | 69 | CATAGATGACCAAGCTTGGCAGG | 8 |
| GGACCACCGTGAGAGTTGCAAGG | 68 | GGTCCCAGATCCAAGCACAGAGG | 53 |
| CTATGACACCACATTGGCATCGG | 67 | ACATGTTCTTCCTGTGATAGTGG | 41 |
| ACATACTTCATTACTGTCACAGG | 66 | GATAACTCTGTCCATTTCCGTGG | 29 |
| TCCACGGCTGTTTCTGAACAAGG | 65 | AGGACACTTGATTCTGTCACTGG | 40 |
| AGGAGAGTTGATTCCATCACTGG | 64 | TCTCCTCACAACTCCCACGGTGG | 70 |
| GTGGTCCTATTATGTTGATGTGG | 64 | TGACACCACATCAACATAATAGG | 69 |
| CTTGGAACCGGGGCCACTGTGGG | 62 | ACAGAGGCTCCTTCTGAACAAGG | 52 |
| GGACCACCGTGGGAGTTGTGAGG | 60 | ACAGAGCTTCCTTCTGAAGAAGG | 46 |
| CACCAACGGAGCAAAGCCCCGGG | 60 | GTTGGTGCTGAAATTCAAAGAGG | 34 |
| CAAGGACTAATCTCAGCATCTGG | 59 | TAGTCCTTGGTGTTATACCATGG | 68 |
| TTCTTACCTTGTTCAGAAGGAGG | 59 | CTAGGGCCTCCTTCTGAACAAGG | 64 |
| GGCTCCTTACCTTGTTCAGAAGG | 59 | TGAACAAGGTAAGGAGCCTGTGG | 39 |
| CTGTCACCAGGCATTGTGTCAGG | 58 | AAGTGTCCTTGCAACTCTCACGG | 34 |
| CTGTGTCTGAGCATCGCGTCAGG | 58 | CTCAGACACAGAAGGGACTGTGG | 33 |
| AAGCCTCTGTGCTTGGATCTGGG | 57 | AGGCTTCTTCIGAAGAAGGTAGG | 55 |
| GCACCAACGGAGCAAAGCCCCGG | 57 | GTTGGTGCTGAAATTCAAAGAGG | 34 |
| CACCAACTGAAAACAGCACTGGG | 56 | ACCCCAGTGCTGTTTTCAGTTGG | 60 |
| GTTGTCCTCTGATGCCAGTGTGG | 56 | CAACAGAATATTATCCAAATGGG | 52 |
| CGGATCGGTTGTGTAACACCAGG | 56 | AACCGATCCGTGTGTGAGGTGGG | 40 |
| CTTGTTCAGAAGGAGGCCCTAGG | 54 | CTAGGGCCTCCTTCTGAACAAGG | 64 |
| GGAGCAAAGCCCCACAGTCCAGG | 53 | GTTGGTGCTGAAATTCAAAGAGG | 34 |
| CTTGTTCAAAAAAAGCCTCTAGG | 52 | CTAGAGGCTTTTTTTGAACAAGG | 57 |
| ACAGAGGCTCCTTCTGAACAAGG | 52 | GCTTGGAACCGGGGCCACTGTGG | 50 |
| CTTGGAACCTGGATGACAGTCGG | 51 | CTAGGGCCTCCTTCTGAACAAGG | 64 |
| CCATGGATCCCAATGTCAGATGG | 51 | CAAGGACTAATCTCAGCATCTGG | 59 |
| CTTGGAGCCAGAATAACATTCGG | 51 | CTAGAGGCTTTTTTTGAACAAGG | 57 |
| GTGGTTCTCTGATGCCAGTGTGG | 51 | CCACAGAATACTACCCAAATGGG | 41 |
| GAGGCCCTAGGCTTGGAACCTGG | 48 | CTAGGGCCTCCTTCTGAACAAGG | 64 |
| TCAGAAGGAGGCCCTAGGCTTGG | 48 | CTAGGGCCTCCTTCTGAACAAGG | 64 |
| CCATCTGACATTGGGATCCATGG | 48 | CATGGATCCCAATGTCAGATGGG | 53 |
| TGAAAACAGCACTGGGGTCCAGG | 47 | ACCCCAGTGCTGTTTTCAGTTGG | 60 |
| ACAACAGAATATTATCCAAATGG | 47 | GTTGTCCTCTGATGCCAGTGTGG | 56 |
| ACAGAGCTTCCTTCTGAAGAAGG | 46 | GCTTGGAACTGGGACCACCGTGG | 60 |
| ACAGAGGCTTCTTCTGAAGAAGG | 46 | AAGCCTCTGTGCTTGGATCTGGG | 57 |
| AGGACACTCGATTCTGTCACTGG | 46 | CACAACTCCCACAGTGGCCCCGG | 45 |
| GGAGCAAAGCCCCGGGGTCCAGG | 46 | GTTGGTGCTGAAATTCAAAGAGG | 34 |
| GGAAGCTCTGTGCTTGGAACTGG | 43 | ACAGAGCTTCCTTCTGAAGAAGG | 46 |
| ATGCCAGTGTGGTGTCATAGAGG | 42 | ACAACAGAATATTATCCAAATGG | 47 |
| ATGCCAGTGTGGTGTCATAGAGG | 42 | ACCACAGAATACTACCCAAATGG | 38 |
| TCGCCCTTGGTGTTACACCATGG | 41 | CAAGGGCGAATCTCAGCATCTGG | 23 |
| TCGCCCTTGGTGTTACACCATGG | 41 | CAAGGGCGAATCTCAGCATCTGG | 23 |
| ACCACAGAATACTACCCAAATGG | 38 | GTGGTTCTCTGATGCCAGTGTGG | 51 |
| TTTCTGGGGTCCGACTATGCTGG | 38 | TGACACCACACCAGCATAGTCGG | 46 |
| GAACAACAGTGGGAGTCTCTAGG | 38 | TGTTCCAGTTCCAAGCATGGAGG | 37 |
| CATGGATCCCAGTGTCAGATGGG | 38 | GATCCATGGTATAACACCAAGGG | 36 |
| TGTTGTTCCAGTTCCAAGCATGG | 37 | GAACAACAGTGGGAGTCTCTAGG | 38 |
| TCTTTGAATTTCAGCACCAACGG | 37 | GTTGGTGCTGAAATTCAAAGAGG | 34 |
| TCTTTGAATTTCAGCACCAACGG | 37 | GTTGGTGCTGAAATTCAAAGAGG | 34 |
| AGCCTCTAGGCTTGGAATCGGGG | 37 | GGCTCCTTCTGAACAAGGTAAGG | 12 |
| TCAGAAGGAAGCTCTGTGCTTGG | 36 | ACAGAGCTTCCTTCTGAAGAAGG | 46 |
| GATCCCTCGTATAACAATAAGGG | 36 | GAGGGATCCCAGTGTCAGGTGGG | 18 |
| CTTGGAACTGGAACAACAGTGGG | 36 | ATGGAGGCTCATTCTGAAGCAGG | 0 |
| GGACACTTGATTCTGTCACCAGG | 35 | TGTCCTTGCAACTCTCACGGTGG | 83 |
| GGGGTCCGACTATGCTGGTGTGG | 35 | TGACACCACACCAGCATAGTCGG | 46 |
| ATCCCTCGTATAACAATAAGGGG | 34 | GAGGGATCCCAGTGTCAGGTGGG | 18 |
| GAAGCTCTGTGCTTGGAACTGGG | 32 | ACAGAGCTTCCTTCTGAAGAAGG | 46 |
| TCAAAAAAAGCCTCTAGGCTTGG | 29 | CTAGAGGCTTTTTTTGAACAAGG | 57 |
| CAAGGGCGAATCTCAGCATCTGG | 23 | TCGCCCTTGGTGTTATACCATGG | 61 |
| GGAGCCTCTAGGCTTGGAATCGG | 23 | TGAACAAGGTAAGGAGCCTGTGG | 39 |
| CAAGGGCGAATCTCAGCATCTGG | 23 | ACACCATGGATCCCAGTGTCAGG | 27 |
| CAAGGGCGAATCTCAGCATCTGG | 23 | ACACCATGGATCCCAGTGTCAGG | 27 |
| GAGCCTCTAGGCTTGGAATCGGG | 23 | GGCTCCTTCTGAACAAGGTAAGG | 12 |
| TTTCAGCACCGACTGAGCAGAGG | 22 | ACCCCAGGCCTCTGCTCAGTCGG | 15 |
| CACCGACTGAGCAGAGGCCTGGG | 22 | ACCCCAGGCCTCTGCTCAGTCGG | 15 |
| TTTCAGCACCGACTGAGCAGAGG | 22 | ACCCCAGGCCTCTGCTCAGTCGG | 15 |
| CACCGACTGAGCAGAGGCCTGGG | 22 | ACCCCAGGCCTCTGCTCAGTCGG | 15 |
| CGAGGGATCCCAGTGTCAGGTGG | 20 | TAACAATAAGGGGCTGCCACAGG | 75 |
| GAGGGATCCCAGTGTCAGGTGGG | 18 | TAACAATAAGGGGCTGCCACAGG | 75 |
| ACCGACTGAGCAGAGGCCTGGGG | 17 | ACCCCAGGCCTCTGCTCAGTCGG | 15 |
| ACCGACTGAGCAGAGGCCTGGGG | 17 | ACCCCAGGCCTCTGCTCAGTCGG | 15 |
| GCAGGTTCTTCCAGTGACAGTGG | 17 | CTGGAAGAACCTGCCAAGCTTGG | 8 |
| GCAGGTTCTTCCAGTGACAGTGG | 17 | CTGGAAGAACCTGCCAAGCTTGG | 8 |
| GCACCGACTGAGCAGAGGCCTGG | 16 | ACCCCAGGCCTCTGCTCAGTCGG | 15 |
| GCACCGACTGAGCAGAGGCCTGG | 16 | ACCCCAGGCCTCTGCTCAGTCGG | 15 |
| TTTCAGCACCGACTGAGCAAAGG | 15 | CGGTGCTGAAATGAAAACACGGG | 16 |
| TTTCAGCACCGACTGAGCAAAGG | 15 | CGGTGCTGAAATGAAAACACGGG | 16 |
| TTTCAGCACCGACTGAGCAAAGG | 15 | CGGTGCTGAAATGAAAACACGGG | 16 |
| TTTCAGCACCGACTGAGCAAAGG | 15 | CGGTGCTGAAATGAAAACACGGG | 16 |
| TTTCAGCACCGACTGAGCAAAGG | 15 | CGGTGCTGAAATGAAAACACGGG | 16 |
| CACCGACTGAGCAAAGGCCTGGG | 14 | CGGTGCTGAAATGAAAACACGGG | 16 |
| CACCGACTGAGCAAAGGCCTGGG | 14 | CGGTGCTGAAATGAAAACACGGG | 16 |
| CACCGACTGAGCAAAGGCCTGGG | 14 | CGGTGCTGAAATGAAAACACGGG | 16 |
| CACCGACTGAGCAAAGGCCTGGG | 14 | CGGTGCTGAAATGAAAACACGGG | 16 |
| CACCGACTGAGCAAAGGCCTGGG | 14 | CGGTGCTGAAATGAAAACACGGG | 16 |
| GGTAACAGTCGGAGGCGCGACGG | 14 | TACCCCGGTTCCAAGCCTAGAGG | 11 |
| GGTAACAGTCGGAGGCGCGACGG | 14 | TACCCCGGTTCCAAGCCTAGAGG | 11 |
| GGTAACAGTCGGAGGCGCGACGG | 14 | TACCCCGGTTCCAAGCCTAGAGG | 11 |
| GGTAACAGTCGGAGGCGCGACGG | 14 | TACCCCGGTTCCAAGCCTAGAGG | 11 |
| GGTAACAGTCGGAGGCGCGACGG | 14 | TACCCCGGTTCCAAGCCTAGAGG | 11 |
| GGTAACAGTCGGAGGCGCGACGG | 14 | TACCCCGGTTCCAAGCCTAGAGG | 11 |
| GGTAACAGTCGGAGGCGCGACGG | 14 | TACCCCGGTTCCAAGCCTAGAGG | 11 |
| TGGATTCCTGCAGTAGTTCCTGG | 13 | CAGATGCTGAGATTAGTCCTTGG | 68 |
| TGGATTCCTGCAGTAGTTCCTGG | 13 | CAGATGCTGAGATTCGCCCTTGG | 29 |
| TGGATTCCTGCAGTAGTTCCTGG | 13 | CAGATGCTGAGATTCGCCCTTGG | 29 |
| TGGATTCCTGCAGTAGTTCCTGG | 13 | CAGATGCTGAGATTCGCCCTTGG | 29 |
| GCACCGACTGAGCAAAGGCCTGG | 13 | CGGTGCTGAAATGAAAACACGGG | 16 |
| GCACCGACTGAGCAAAGGCCTGG | 13 | CGGTGCTGAAATGAAAACACGGG | 16 |
| GCACCGACTGAGCAAAGGCCTGG | 13 | CGGTGCTGAAATGAAAACACGGG | 16 |
| GCACCGACTGAGCAAAGGCCTGG | 13 | CGGTGCTGAAATGAAAACACGGG | 16 |
| GCACCGACTGAGCAAAGGCCTGG | 13 | CGGTGCTGAAATGAAAACACGGG | 16 |
| AGTTCTTCCTGTGACAGTGGTGG | 13 | CAGGAAGAACTTGCCAAGCTTGG | 12 |
| TCTCCACCACTGTCACAGGAAGG | 12 | AGAATGTGCCTCGATAACTCTGG | 86 |
| GGATCCCTCGTATAACAATAAGG | 12 | GAGGGATCCCAGTGTCAGGTGGG | 18 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GGGGTCCGACTATGCGAGTGTGG | 12 | TGACACCACACTCGCATAGTCGG | 12 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GGGGTCCGACTATGCGAGTGTGG | 12 | TGACACCACACTCGCATAGTCGG | 12 |
| GAGGGATCCCGGTGTCAGGTGGG | 12 | GGATCCCTCGTATAACAATAAGG | 12 |
| GCAGGTCCTTCCTGTGACAGTGG | 12 | CAGGAAGGACCTGCCAAGCTTGG | 9 |
| TGGATTCCTGCAGTAGTTCTCGG | 11 | CAGATTCTGGGAAACAACCCTGG | 38 |
| TGAGCAGAGGCCTGGGGTGCAGG | 10 | ACCCCAGGCCTCTGCTCAGTCGG | 15 |
| TGAGCAGAGGCCTGGGGTGCAGG | 10 | ACCCCAGGCCTCTGCTCAGTCGG | 15 |
| GCAAGTTCTTCCTGTGACAGTGG | 10 | CAGGAAGAACTTGCCAAGCTTGG | 12 |
| CAGGAAGGACCTGCCAAGCTTGG | 9 | GGTCCTTCCTGTGACAGTGGTGG | 8 |
| GCAGGTTCTTCCTGTGACAGTGG | 9 | CAGGAAGAACCTGCCAAGCTTGG | 7 |
| GCAGGTTCTTCCTGTGACAGTGG | 9 | CAGGAAGAACCTGCCAAGCTTGG | 7 |
| GCAGGTTCTTCCTGTGACAGTGG | 9 | CAGGAAGAACCTGCCAAGCTTGG | 7 |
| GCAGGTTCTTCCTGTGACAGTGG | 9 | CAGGAAGAACCTGCCAAGCTTGG | 7 |
| GCAGGTTCTTCCTGTGACAGTGG | 9 | CAGGAAGAACCTGCCAAGCTTGG | 7 |
| CATAGATGACCAAGCTTGGCAGG | 8 | TGACACCACATCAACATAATAGG | 69 |
| CATAGATGACCAAGCTTGGCAGG | 8 | TGACACCACACTCGCATAGTCGG | 12 |
| CATAGATGACCAAGCTTGGCAGG | 8 | TGACACCACACTCGCATAGTCGG | 12 |
| CATAGATGACCAAGCTTGGCAGG | 8 | TGACACCACACTCGCATAGTCGG | 12 |
| CATAGATGACCAAGCTTGGCAGG | 8 | TGACACCACACTCGCATAGTCGG | 12 |
| CTGGAAGAACCTGCCAAGCTTGG | 8 | GGTTCTTCCAGTGACAGTGGTGG | 12 |
| CATAGATGACCAAGCTTGGCAGG | 8 | TGACACCACACTCGCATAGTCGG | 12 |
| CATAGATGACCAAGCTTGGCAGG | 8 | TGACACCACACTCGCATAGTCGG | 12 |
| CATAGATGACCAAGCTTGGCAGG | 8 | TGACACCACACTCGCATAGTCGG | 12 |
| CATAGATGACCAAGCTTGGCAGG | 8 | TGACACCACACTCGCATAGTCGG | 12 |
| CTGGAAGAACCTGCCAAGCTTGG | 8 | GGTTCTTCCAGTGACAGTGGTGG | 12 |
| CAGGAAGAACCTGCCAAGCTTGG | 7 | GATAACTCTGTCCATTTCCGTGG | 29 |
| CAGGAAGAACCTGCCAAGCTTGG | 7 | GGTTCTTCCTGTGACAGTGGTGG | 9 |
| CAGGAAGAACCTGCCAAGCTTGG | 7 | GGTTCTTCCTGTGACAGTGGTGG | 9 |
| CAGGAAGAACCTGCCAAGCTTGG | 7 | GGTTCTTCCTGTGACAGTGGTGG | 9 |
| CAGGAAGAACCTGCCAAGCTTGG | 7 | GGTTCTTCCTGTGACAGTGGTGG | 9 |
| CAGGAAGAACCTGCCAAGCTTGG | 7 | GGTTCTTCCTGTGACAGTGGTGG | 9 |

PAM sequence is shown in bolded and underlined text; corresponding spacers may have sequences identical to protospacer sequences (“U” is substituted for “T” if spacer sequence is RNA)

Each guide nucleic acid may include a portion that is recognized by and binds to the gene editor (e.g., the nickase). The portion of the guide nucleic acid that recognizes and binds the gene editor may be referred to herein as the “scaffold” region. The scaffold region may form one or more stem loop structures that are recognized by the gene editor. A length of a loop and a stem can vary. In one or more embodiments, a loop can range from about 3 to about 10 nucleotides in length. In one or more embodiments, a stem can range from about 6 to about 20 nucleotides in length. A stem can comprise one or more bulges of 1 to 10 nucleotides or about 10 nucleotides. In one or more embodiments, the overall length of a second region can range from about 16 to 60 nucleotides in length. In one or more embodiments, a loop can be about 4 nucleotides in length. In one or more embodiments, a stem can be about 12 nucleotides in length. The scaffold region may include a region that does not form a stem loop structure. This portion lacking substantial secondary structure may have any suitable length, such as ranging from about 3 to about 100 nucleotides in length. The portion of the scaffold region lacking substantial secondary structure may be located at the 3′ end of the guide nucleic acid. In some embodiments, the scaffold region comprises a tracr sequence. In some embodiments, the scaffold region consists of, or consists essentially of, a tracr sequence.

In one or more embodiments, the sequence of the scaffold region includes one of the following sequences:

5′-GUUUUAGAGCUAGAAAUAGCAAGUUAAAAUAAGGCUAGUCCGUUAUC
AACUUGAAAAAGUGGCACCGAGUCGGUGC-3′,
or
5′-GUUUGAGAGCUAUGCUGGAAACAGCAUAGCAAGUUCAAAUAAGGCUA
GUCCGUUAUCAACUUGAAAAAGUGGCACCGAGUCGGUGC-3′.

In one or more embodiments, the scaffold region may have a sequence that is at least 75%, at least 85%, at least 90%, or at least 95% identical to one of the sequences herein. It should be apparent to one skilled in the art that the appropriate scaffold region sequence and/or length may change depending on a number of factors, such as the editing protein used. In one or more embodiments, the scaffold region may include DNA and/or RNA nucleotides. The scaffold region may be modified as described herein.

In one or more embodiments, the sequence of the scaffold region comprises, consists essentially of, or consists of: 5′-mGUUUUAGmAmGmCmUmAGmAmAmAmUmAmGmCmAmAGUUmAAmAAmUAmAm GmGmCmUmAGUmCmCGUUAmUmCAAmCmUmUGmAmAmAmAmAmGmUmGGmCm AmCmCmGmAmGmUmCmGmGmUmGmC 3′, where m of mN is 2′-O-methyl ribose.

In one or more embodiments, a guide oligonucleotide may include an unstructured or a structured RNA motif, designed to be capable of preventing degradation of the nucleic acid. The RNA motif may be located at the 3′ end of the nucleic acid. The nucleic acid may include any suitable RNA motif, such as RNA motifs including the sequences listed below.

In one or more embodiments, a guide oligonucleotide includes an RNA motif on the 5′ end of the nucleic acid. In one or more embodiments, the guide oligonucleotide includes an RNA motif on the 5′ end and the 3′ end. If two or more RNA stabilizing motifs are incorporated into a guide oligonucleotide, they may have the same sequence, or they may have different sequences.

A guide oligonucleotide may include an unstructured RNA motif that comprises, consists essentially of, or consists of the sequence 5′-UUU-3′. In embodiments, the unstructured motif comprises modifications as follows: 5′-*mU*mU*mU-3′, where “mU*” indicates a phosphorothioated 2′-O-methyl uracil base, and “mU” indicates a 2′-O-methyl uracil base.

A guide oligonucleotide may include a tevopreQ1 motif. A tevopreQ1 motif may be modified from a prequeosinel-1 riboswitch aptamer. A guide oligonucleotide may include a tevopreQ1 motif that comprises, consists essentially of, or consists of the sequence 5′-CGCGGUUCUAUCUAGUUACGCGUUAAACCAACUAGAA-3′.

A guide oligonucleotide may include a mpknot motif A mpknot motif may be modified from a frameshifting pseudoknot from Moloney murine leukemia virus. A guide oligonucleotide may include a mpknot motif that comprises, consists essentially of, or consists of the sequence

5′-GGGUCAGGAGCCCCCCCCCUGAACCCAGGAUAACCCUCAAAGUCGGG
GGGCAACCC-3′.

In some embodiments, a gRNA consists of, or consists essentially of, a sequence as follows: 5′-mN*mN*mN*NNNNNNNNNNNNNNNNNmGU U UUAGmAmGmCmUmAGmAmAmAmU mAmGmCmAmAGUUmAAmAAmUAmAmGmGmCmUmAGUmCmCGUUAmUmCAAmC mUmUGmAmAmAmAmAmGmUmGGmCmAmCmCmGmAmGmUmCmGmGmUmGmCmU *mU*mU*mU-3′, where N refers to any nucleotide, m of mN is 2′-O-methyl ribose, and * refers to a phosphorothioate linkage. The first 20 nucleotides may correspond to a spacer sequence. The spacer sequence may be identical to or substantially identical to a protospacer listed in Table 2 or Table 5.

A guide nucleic acid of a dual nickase editing system may have any suitable length. The length may depend on the CRISPR Cas component of the gene editor system and components used. For example, different Cas proteins from different bacterial species have varying optimal spacer sequence lengths. Accordingly, the spacer sequence may comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In one or more embodiments, the spacer sequence comprises 15 to 24 nucleotides in length. In embodiments, the spacer sequence comprises 18 to 24 nucleotides in length. In one or more embodiments, the spacer sequence comprises 19 to 21 nucleotides in length. In one or more embodiments, the spacer sequence comprises 20 nucleotides in length +/−1 nucleotide, +/−2 nucleotides, or +/−3 nucleotides.

In one or more embodiments, each guide nucleic acid includes a spacer sequence and otherwise conforms to a conventional 100-nucleotide Streptococcus pyogenes CRISPR gRNA sequence.

Each guide nucleic acid (e.g., guide RNA) may be about 15-300 nucleotides long and may comprise a sequence of at least 10 contiguous nucleotides that is substantially identical to a protospacer sequence at a targeted DNA site. In one or more embodiments, the 3′ end of the protospacer sequence is immediately adjacent to a canonical PAM sequence (e.g., NGG). In one or more embodiments, the 3′ end of the protospacer sequence is not immediately adjacent to a canonical PAM sequence. In one or more embodiments, the 3′ end of the protospacer sequence is immediately adjacent to an NAG, NGA, NGC, NGT, or other non-NGG sequence.

In one or more embodiments, a guide nucleic acid includes DNA nucleotides. In one or more embodiments, a guide nucleic acid includes DNA and RNA. In one or more embodiments, a guide nucleic acid is an RNA, also referred to herein as a guide RNA or gRNA. In one or more embodiments, a guide nucleic acid is a modified nucleic acid. As used herein, a modified nucleic acid is a nucleic acid comprising at least one modified nucleobase, sugar, or backbone (e.g., linkage) moiety.

In one or more embodiments, a guide nucleic acid may be synthesized. The guide nucleic acid may comprise a spacer sequence configured to hybridize to the complementary sequence on a target strand under, for example, conditions within a cell. In embodiments, the guide nucleic acid comprises a spacer sequence that is homologous to a protospacer sequence in an LPA gene. The guide nucleic acid may comprise a spacer sequence that is identical to or substantially identical to a protospacer sequence. In one or more embodiments, the guide nucleic acid comprises a spacer having a sequence identical to or substantially identical to a protospacer set forth in Table 2 or Table 5.

In one or more embodiments, the guide oligonucleotide may have a sequence set forth in Table 3 in which zero, one, or more than one nucleotide may be modified. Table 3. Example sequences of guide oligonucleotides Sequence

TABLE 3
Example sequences of guide oligonucleotides
Sequence

CUGUCACCAGGCAUUGUGUCGUUUUAGAGCUAGAAAUAGCAAGUUAAAAUAAGGCUAGUCCGU
UAUCAACUUGAAAAAGUGGCACCGAGUCGGUGCUUUU
GUAGUAGCAGUCCUGUACCCGUUUUAGAGCUAGAAAUAGCAAGUUAAAAUAAGGCUAGUCCGU
UAUCAACUUGAAAAAGUGGCACCGAGUCGGUGCUUUU
AGGACACUCGAUUCUGUCACGUUUUAGAGCUAGAAAUAGCAAGUUAAAAUAAGGCUAGUCCGU
UAUCAACUUGAAAAAGUGGCACCGAGUCGGUGCUUUU
CUGUCACUGGACAUUGUGUCGUUUUAGAGCUAGAAAUAGCAAGUUAAAAUAAGGCUAGUCCGU
UAUCAACUUGAAAAAGUGGCACCGAGUCGGUGCUUUU
GGAGCAAAGCCCCACAGUCCGUUUUAGAGCUAGAAAUAGCAAGUUAAAAUAAGGCUAGUCCGU
UAUCAACUUGAAAAAGUGGCACCGAGUCGGUGCUUUU
GGAGCAAAGCCCCGGGGUCCGUUUUAGAGCUAGAAAUAGCAAGUUAAAAUAAGGCUAGUCCGU
UAUCAACUUGAAAAAGUGGCACCGAGUCGGUGCUUUU
CUUGGAACUGGGACCACCGUGUUUUAGAGCUAGAAAUAGCAAGUUAAAAUAAGGCUAGUCCGU
UAUCAACUUGAAAAAGUGGCACCGAGUCGGUGCUUUU
AUGCCAGUGUGGUGUCAUAGGUUUUAGAGCUAGAAAUAGCAAGUUAAAAUAAGGCUAGUCCGU
UAUCAACUUGAAAAAGUGGCACCGAGUCGGUGCUUUU
GGAGCCAGAAUAACAUUCGGGUUUUAGAGCUAGAAAUAGCAAGUUAAAAUAAGGCUAGUCCGU
UAUCAACUUGAAAAAGUGGCACCGAGUCGGUGCUUUU
CAGAUGCUGAGAUUAGUCCUGUUUUAGAGCUAGAAAUAGCAAGUUAAAAUAAGGCUAGUCCGU
UAUCAACUUGAAAAAGUGGCACCGAGUCGGUGCUUUU
UGACACCACAUUGGCAUCGGGUUUUAGAGCUAGAAAUAGCAAGUUAAAAUAAGGCUAGUCCGU
UAUCAACUUGAAAAAGUGGCACCGAGUCGGUGCUUUU
CAUAGAUGACCAAGAUUGACGUUUUAGAGCUAGAAAUAGCAAGUUAAAAUAAGGCUAGUCCGU
UAUCAACUUGAAAAAGUGGCACCGAGUCGGUGCUUUU
CCAUCACUGGACAUUGCGUCGUUUUAGAGCUAGAAAUAGCAAGUUAAAAUAAGGCUAGUCCGU
UAUCAACUUGAAAAAGUGGCACCGAGUCGGUGCUUUU
CUGCAUCUGAGCAUCGUGUCGUUUUAGAGCUAGAAAUAGCAAGUUAAAAUAAGGCUAGUCCGU
UAUCAACUUGAAAAAGUGGCACCGAGUCGGUGCUUUU
AAACAGCCGUGGACGUCGCAGUUUUAGAGCUAGAAAUAGCAAGUUAAAAUAAGGCUAGUCCGU
UAUCAACUUGAAAAAGUGGCACCGAGUCGGUGCUUUU
ACAGAGGCUCCUUCUGAACAGUUUUAGAGCUAGAAAUAGCAAGUUAAAAUAAGGCUAGUCCGU
UAUCAACUUGAAAAAGUGGCACCGAGUCGGUGCUUUU
AUGCCAGUGUGGUGUCAUAGGUUUUAGAGCUAGAAAUAGCAAGUUAAAAUAAGGCUAGUCCGU
UAUCAACUUGAAAAAGUGGCACCGAGUCGGUGCUUUU
CUAUGACACCACAUUGGCAUGUUUUAGAGCUAGAAAUAGCAAGUUAAAAUAAGGCUAGUCCGU
UAUCAACUUGAAAAAGUGGCACCGAGUCGGUGCUUUU
AAUAACAUUCGGAGGGACGAGUUUUAGAGCUAGAAAUAGCAAGUUAAAAUAAGGCUAGUCCGU
UAUCAACUUGAAAAAGUGGCACCGAGUCGGUGCUUUU
GAACAACAGUGGGAGUCUCUGUUUUAGAGCUAGAAAUAGCAAGUUAAAAUAAGGCUAGUCCGU
UAUCAACUUGAAAAAGUGGCACCGAGUCGGUGCUUUU
AAGCCUCUGUGCUUGGAUCUGUUUUAGAGCUAGAAAUAGCAAGUUAAAAUAAGGCUAGUCCGU
UAUCAACUUGAAAAAGUGGCACCGAGUCGGUGCUUUU
CUAGGGCCUCCUUCUGAACAGUUUUAGAGCUAGAAAUAGCAAGUUAAAAUAAGGCUAGUCCGU
UAUCAACUUGAAAAAGUGGCACCGAGUCGGUGCUUUU
UGUUGUUCCAGUUCCAAGCAGUUUUAGAGCUAGAAAUAGCAAGUUAAAAUAAGGCUAGUCCGU
UAUCAACUUGAAAAAGUGGCACCGAGUCGGUGCUUUU
CUUGGAACCGGGGCCACUGUGUUUUAGAGCUAGAAAUAGCAAGUUAAAAUAAGGCUAGUCCGU
UAUCAACUUGAAAAAGUGGCACCGAGUCGGUGCUUUU
ACAUACUUCAUUACUGUCACGUUUUAGAGCUAGAAAUAGCAAGUUAAAAUAAGGCUAGUCCGU
UAUCAACUUGAAAAAGUGGCACCGAGUCGGUGCUUUU
UACCCCGGUUCCAAGCCUAGGUUUUAGAGCUAGAAAUAGCAAGUUAAAAUAAGGCUAGUCCGU
UAUCAACUUGAAAAAGUGGCACCGAGUCGGUGCUUUU
CAGGAAGGACCUGCCAAGCUGUUUUAGAGCUAGAAAUAGCAAGUUAAAAUAAGGCUAGUCCGU
UAUCAACUUGAAAAAGUGGCACCGAGUCGGUGCUUUU
ACAACAGAAUAUUAUCCAAAGUUUUAGAGCUAGAAAUAGCAAGUUAAAAUAAGGCUAGUCCGU
UAUCAACUUGAAAAAGUGGCACCGAGUCGGUGCUUUU
GGACCACCGUGAGAGUUGCAGUUUUAGAGCUAGAAAUAGCAAGUUAAAAUAAGGCUAGUCCGU
UAUCAACUUGAAAAAGUGGCACCGAGUCGGUGCUUUU
AGGAGAGUUGAUUCCAUCACGUUUUAGAGCUAGAAAUAGCAAGUUAAAAUAAGGCUAGUCCGU
UAUCAACUUGAAAAAGUGGCACCGAGUCGGUGCUUUU
GUGGUUCUCUGAUGCCAGUGGUUUUAGAGCUAGAAAUAGCAAGUUAAAAUAAGGCUAGUCCGU
UAUCAACUUGAAAAAGUGGCACCGAGUCGGUGCUUUU
ACCACAGAAUACUACCCAAAGUUUUAGAGCUAGAAAUAGCAAGUUAAAAUAAGGCUAGUCCGU
UAUCAACUUGAAAAAGUGGCACCGAGUCGGUGCUUUU
ACAGAGGCUUCUUCUGAAGAGUUUUAGAGCUAGAAAUAGCAAGUUAAAAUAAGGCUAGUCCGU
UAUCAACUUGAAAAAGUGGCACCGAGUCGGUGCUUUU
UAGUCCUUGGUGUUAUACCAGUUUUAGAGCUAGAAAUAGCAAGUUAAAAUAAGGCUAGUCCGU
UAUCAACUUGAAAAAGUGGCACCGAGUCGGUGCUUUU
CACCAACGGAGCAAAGCCCCGUUUUAGAGCUAGAAAUAGCAAGUUAAAAUAAGGCUAGUCCGU
UAUCAACUUGAAAAAGUGGCACCGAGUCGGUGCUUUU
GUUGUCCUCUGAUGCCAGUGGUUUUAGAGCUAGAAAUAGCAAGUUAAAAUAAGGCUAGUCCGU
UAUCAACUUGAAAAAGUGGCACCGAGUCGGUGCUUUU
UGACACCACAUCAACAUAAUGUUUUAGAGCUAGAAAUAGCAAGUUAAAAUAAGGCUAGUCCGU
UAUCAACUUGAAAAAGUGGCACCGAGUCGGUGCUUUU
CGGAUCGGUUGUGUAACACCGUUUUAGAGCUAGAAAUAGCAAGUUAAAAUAAGGCUAGUCCGU
UAUCAACUUGAAAAAGUGGCACCGAGUCGGUGCUUUU
UCGCCCUUGGUGUUACACCAGUUUUAGAGCUAGAAAUAGCAAGUUAAAAUAAGGCUAGUCCGU
UAUCAACUUGAAAAAGUGGCACCGAGUCGGUGCUUUU
CAGGAAGAACCUGCCAAGCUGUUUUAGAGCUAGAAAUAGCAAGUUAAAAUAAGGCUAGUCCGU
UAUCAACUUGAAAAAGUGGCACCGAGUCGGUGCUUUU
GGUAAUAGUUGGAGGCGCGAGUUUUAGAGCUAGAAAUAGCAAGUUAAAAUAAGGCUAGUCCGU
UAUCAACUUGAAAAAGUGGCACCGAGUCGGUGCUUUU
ACUGUGGGGCUUUGCUCCGUGUUUUAGAGCUAGAAAUAGCAAGUUAAAAUAAGGCUAGUCCGU
UAUCAACUUGAAAAAGUGGCACCGAGUCGGUGCUUUU
CUGUGUCUGAGCAUCGCGUCGUUUUAGAGCUAGAAAUAGCAAGUUAAAAUAAGGCUAGUCCGU
UAUCAACUUGAAAAAGUGGCACCGAGUCGGUGCUUUU
CAUGGAUCCCAGUGUCAGAUGUUUUAGAGCUAGAAAUAGCAAGUUAAAAUAAGGCUAGUCCGU
UAUCAACUUGAAAAAGUGGCACCGAGUCGGUGCUUUU
GCACCAACGGAGCAAAGCCCGUUUUAGAGCUAGAAAUAGCAAGUUAAAAUAAGGCUAGUCCGU
UAUCAACUUGAAAAAGUGGCACCGAGUCGGUGCUUUU
UGAAAACAGCACUGGGGUCCGUUUUAGAGCUAGAAAUAGCAAGUUAAAAUAAGGCUAGUCCGU
UAUCAACUUGAAAAAGUGGCACCGAGUCGGUGCUUUU
AACUCAAGGUUGUACCCAUUGUUUUAGAGCUAGAAAUAGCAAGUUAAAAUAAGGCUAGUCCGU
UAUCAACUUGAAAAAGUGGCACCGAGUCGGUGCUUUU
GGGAUCCUCCGAUGCCAAUGGUUUUAGAGCUAGAAAUAGCAAGUUAAAAUAAGGCUAGUCCGU
UAUCAACUUGAAAAAGUGGCACCGAGUCGGUGCUUUU
CUACUUAGAAGCUGAAACGUGUUUUAGAGCUAGAAAUAGCAAGUUAAAAUAAGGCUAGUCCGU
UAUCAACUUGAAAAAGUGGCACCGAGUCGGUGCUUUU
GGCUCCUUACCUUGUUCAGAGUUUUAGAGCUAGAAAUAGCAAGUUAAAAUAAGGCUAGUCCGU
UAUCAACUUGAAAAAGUGGCACCGAGUCGGUGCUUUU
CCUACUUAGAAGCUGAAACGGUUUUAGAGCUAGAAAUAGCAAGUUAAAAUAAGGCUAGUCCGU
UAUCAACUUGAAAAAGUGGCACCGAGUCGGUGCUUUU
GGGGUCCGACUAUGCUGGUGGUUUUAGAGCUAGAAAUAGCAAGUUAAAAUAAGGCUAGUCCGU
UAUCAACUUGAAAAAGUGGCACCGAGUCGGUGCUUUU
CACCAACUGAAAACAGCACUGUUUUAGAGCUAGAAAUAGCAAGUUAAAAUAAGGCUAGUCCGU
UAUCAACUUGAAAAAGUGGCACCGAGUCGGUGCUUUU
UUUCUGGGGUCCGACUAUGCGUUUUAGAGCUAGAAAUAGCAAGUUAAAAUAAGGCUAGUCCGU
UAUCAACUUGAAAAAGUGGCACCGAGUCGGUGCUUUU
UCUUUGAAUUUCAGCACCAAGUUUUAGAGCUAGAAAUAGCAAGUUAAAAUAAGGCUAGUCCGU
UAUCAACUUGAAAAAGUGGCACCGAGUCGGUGCUUUU
GUGGUCCUAUUAUGUUGAUGGUUUUAGAGCUAGAAAUAGCAAGUUAAAAUAAGGCUAGUCCGU
UAUCAACUUGAAAAAGUGGCACCGAGUCGGUGCUUUU
CCAUCUGACAUUGGGAUCCAGUUUUAGAGCUAGAAAUAGCAAGUUAAAAUAAGGCUAGUCCGU
UAUCAACUUGAAAAAGUGGCACCGAGUCGGUGCUUUU
UCCACGGCUGUUUCUGAACAGUUUUAGAGCUAGAAAUAGCAAGUUAAAAUAAGGCUAGUCCGU
UAUCAACUUGAAAAAGUGGCACCGAGUCGGUGCUUUU
UGGACCACAUGGCUUUGCUCGUUUUAGAGCUAGAAAUAGCAAGUUAAAAUAAGGCUAGUCCGU
UAUCAACUUGAAAAAGUGGCACCGAGUCGGUGCUUUU
UGUCCUUGCAACUCUCACGGGUUUUAGAGCUAGAAAUAGCAAGUUAAAAUAAGGCUAGUCCGU
UAUCAACUUGAAAAAGUGGCACCGAGUCGGUGCUUUU
CAUUAUGGACAGAGUUACCGGUUUUAGAGCUAGAAAUAGCAAGUUAAAAUAAGGCUAGUCCGU
UAUCAACUUGAAAAAGUGGCACCGAGUCGGUGCUUUU
CACAACUCCCACAGUGGCCCGUUUUAGAGCUAGAAAUAGCAAGUUAAAAUAAGGCUAGUCCGU
UAUCAACUUGAAAAAGUGGCACCGAGUCGGUGCUUUU
AAGUGUCCUUGCGACGUCCAGUUUUAGAGCUAGAAAUAGCAAGUUAAAAUAAGGCUAGUCCGU
UAUCAACUUGAAAAAGUGGCACCGAGUCGGUGCUUUU
GUUGGUGCUGAAAUUCAAAGGUUUUAGAGCUAGAAAUAGCAAGUUAAAAUAAGGCUAGUCCGU
UAUCAACUUGAAAAAGUGGCACCGAGUCGGUGCUUUU
GUUGGUGCUGAAAUUCAAAGGUUUUAGAGCUAGAAAUAGCAAGUUAAAAUAAGGCUAGUCCGU
UAUCAACUUGAAAAAGUGGCACCGAGUCGGUGCUUUU
ACAGAGCUUCCUUCUGAAGAGUUUUAGAGCUAGAAAUAGCAAGUUAAAAUAAGGCUAGUCCGU
UAUCAACUUGAAAAAGUGGCACCGAGUCGGUGCUUUU
ACCACAGAAUACUACCCAAAGUUUUAGAGCUAGAAAUAGCAAGUUAAAAUAAGGCUAGUCCGU
UAUCAACUUGAAAAAGUGGCACCGAGUCGGUGCUUUU
CUAGAGGCUUUUUUUGAACAGUUUUAGAGCUAGAAAUAGCAAGUUAAAAUAAGGCUAGUCCGU
UAUCAACUUGAAAAAGUGGCACCGAGUCGGUGCUUUU
UGGAUUCCUGCAGUAGUUCCGUUUUAGAGCUAGAAAUAGCAAGUUAAAAUAAGGCUAGUCCGU
UAUCAACUUGAAAAAGUGGCACCGAGUCGGUGCUUUU
ACAUGUUCUUCCUGUGAUAGGUUUUAGAGCUAGAAAUAGCAAGUUAAAAUAAGGCUAGUCCGU
UAUCAACUUGAAAAAGUGGCACCGAGUCGGUGCUUUU
UGAUACCACACUGGCAUCAGGUUUUAGAGCUAGAAAUAGCAAGUUAAAAUAAGGCUAGUCCGU
UAUCAACUUGAAAAAGUGGCACCGAGUCGGUGCUUUU
AACUCUCCUCACAACUCCCAGUUUUAGAGCUAGAAAUAGCAAGUUAAAAUAAGGCUAGUCCGU
UAUCAACUUGAAAAAGUGGCACCGAGUCGGUGCUUUU
CGUCCCUCCGAAUGUUAUUCGUUUUAGAGCUAGAAAUAGCAAGUUAAAAUAAGGCUAGUCCGU
UAUCAACUUGAAAAAGUGGCACCGAGUCGGUGCUUUU
UGAACAAGGUAAGAAGUCUCGUUUUAGAGCUAGAAAUAGCAAGUUAAAAUAAGGCUAGUCCGU
UAUCAACUUGAAAAAGUGGCACCGAGUCGGUGCUUUU
GCUUGGAACCGGGGCCACUGGUUUUAGAGCUAGAAAUAGCAAGUUAAAAUAAGGCUAGUCCGU
UAUCAACUUGAAAAAGUGGCACCGAGUCGGUGCUUUU
ACAACAGAAUAUUAUCCAAAGUUUUAGAGCUAGAAAUAGCAAGUUAAAAUAAGGCUAGUCCGU
UAUCAACUUGAAAAAGUGGCACCGAGUCGGUGCUUUU
ACAUGUUCUUCCUGUGAUAGGUUUUAGAGCUAGAAAUAGCAAGUUAAAAUAAGGCUAGUCCGU
UAUCAACUUGAAAAAGUGGCACCGAGUCGGUGCUUUU
UAUUCUGGCUCCAAGCCUAGGUUUUAGAGCUAGAAAUAGCAAGUUAAAAUAAGGCUAGUCCGU
UAUCAACUUGAAAAAGUGGCACCGAGUCGGUGCUUUU
UGUUCCAGUUCCAAGCAUGGGUUUUAGAGCUAGAAAUAGCAAGUUAAAAUAAGGCUAGUCCGU
UAUCAACUUGAAAAAGUGGCACCGAGUCGGUGCUUUU
AGGCUUCUUCUGAAGAAGGUGUUUUAGAGCUAGAAAUAGCAAGUUAAAAUAAGGCUAGUCCGU
UAUCAACUUGAAAAAGUGGCACCGAGUCGGUGCUUUU
CUUGGAACCUGGAUGACAGUGUUUUAGAGCUAGAAAUAGCAAGUUAAAAUAAGGCUAGUCCGU
UAUCAACUUGAAAAAGUGGCACCGAGUCGGUGCUUUU
GAACAACAGUGGGAGUCUCUGUUUUAGAGCUAGAAAUAGCAAGUUAAAAUAAGGCUAGUCCGU
UAUCAACUUGAAAAAGUGGCACCGAGUCGGUGCUUUU
ACAGAGGCUCCUUCUGAACAGUUUUAGAGCUAGAAAUAGCAAGUUAAAAUAAGGCUAGUCCGU
UAUCAACUUGAAAAAGUGGCACCGAGUCGGUGCUUUU
GAUAACUCUGUCCAUUUCCGGUUUUAGAGCUAGAAAUAGCAAGUUAAAAUAAGGCUAGUCCGU
UAUCAACUUGAAAAAGUGGCACCGAGUCGGUGCUUUU
GGUAACAGUCGGAGGCGCGAGUUUUAGAGCUAGAAAUAGCAAGUUAAAAUAAGGCUAGUCCGU
UAUCAACUUGAAAAAGUGGCACCGAGUCGGUGCUUUU
GGUCCUUCCUGUGACAGUGGGUUUUAGAGCUAGAAAUAGCAAGUUAAAAUAAGGCUAGUCCGU
UAUCAACUUGAAAAAGUGGCACCGAGUCGGUGCUUUU
GUUGUCCUCUGAUGCCAGUGGUUUUAGAGCUAGAAAUAGCAAGUUAAAAUAAGGCUAGUCCGU
UAUCAACUUGAAAAAGUGGCACCGAGUCGGUGCUUUU
GGUCCCAGAUCCAAGCACAGGUUUUAGAGCUAGAAAUAGCAAGUUAAAAUAAGGCUAGUCCGU
UAUCAACUUGAAAAAGUGGCACCGAGUCGGUGCUUUU
UCUCCUCACAACUCCCACGGGUUUUAGAGCUAGAAAUAGCAAGUUAAAAUAAGGCUAGUCCGU
UAUCAACUUGAAAAAGUGGCACCGAGUCGGUGCUUUU
CCACAGAAUACUACCCAAAUGUUUUAGAGCUAGAAAUAGCAAGUUAAAAUAAGGCUAGUCCGU
UAUCAACUUGAAAAAGUGGCACCGAGUCGGUGCUUUU
GUGGUUCUCUGAUGCCAGUGGUUUUAGAGCUAGAAAUAGCAAGUUAAAAUAAGGCUAGUCCGU
UAUCAACUUGAAAAAGUGGCACCGAGUCGGUGCUUUU
AAGCCUCUGUGCUUGGAUCUGUUUUAGAGCUAGAAAUAGCAAGUUAAAAUAAGGCUAGUCCGU
UAUCAACUUGAAAAAGUGGCACCGAGUCGGUGCUUUU
CAAGGACUAAUCUCAGCAUCGUUUUAGAGCUAGAAAUAGCAAGUUAAAAUAAGGCUAGUCCGU
UAUCAACUUGAAAAAGUGGCACCGAGUCGGUGCUUUU
GUUGGUGCUGAAAUUCAAAGGUUUUAGAGCUAGAAAUAGCAAGUUAAAAUAAGGCUAGUCCGU
UAUCAACUUGAAAAAGUGGCACCGAGUCGGUGCUUUU
CAACAGAAUAUUAUCCAAAUGUUUUAGAGCUAGAAAUAGCAAGUUAAAAUAAGGCUAGUCCGU
UAUCAACUUGAAAAAGUGGCACCGAGUCGGUGCUUUU
CAUAGAUGACCAAGCUUGGCGUUUUAGAGCUAGAAAUAGCAAGUUAAAAUAAGGCUAGUCCGU
UAUCAACUUGAAAAAGUGGCACCGAGUCGGUGCUUUU
AACCGAUCCGUGUGUGAGGUGUUUUAGAGCUAGAAAUAGCAAGUUAAAAUAAGGCUAGUCCGU
UAUCAACUUGAAAAAGUGGCACCGAGUCGGUGCUUUU
CAAGGGCGAAUCUCAGCAUCGUUUUAGAGCUAGAAAUAGCAAGUUAAAAUAAGGCUAGUCCGU
UAUCAACUUGAAAAAGUGGCACCGAGUCGGUGCUUUU
GGUUCUUCCUGUGACAGUGGGUUUUAGAGCUAGAAAUAGCAAGUUAAAAUAAGGCUAGUCCGU
UAUCAACUUGAAAAAGUGGCACCGAGUCGGUGCUUUU
UACCCCGAUUCCAAGCCUAGGUUUUAGAGCUAGAAAUAGCAAGUUAAAAUAAGGCUAGUCCGU
UAUCAACUUGAAAAAGUGGCACCGAGUCGGUGCUUUU
ACAGUCCAGGACUGCUACCAGUUUUAGAGCUAGAAAUAGCAAGUUAAAAUAAGGCUAGUCCGU
UAUCAACUUGAAAAAGUGGCACCGAGUCGGUGCUUUU
CUCAGACACAGAAGGGACUGGUUUUAGAGCUAGAAAUAGCAAGUUAAAAUAAGGCUAGUCCGU
UAUCAACUUGAAAAAGUGGCACCGAGUCGGUGCUUUU
GAUCCAUGGUAUAACACCAAGUUUUAGAGCUAGAAAUAGCAAGUUAAAAUAAGGCUAGUCCGU
UAUCAACUUGAAAAAGUGGCACCGAGUCGGUGCUUUU
GUUGGUGCUGAAAUUCAAAGGUUUUAGAGCUAGAAAUAGCAAGUUAAAAUAAGGCUAGUCCGU
UAUCAACUUGAAAAAGUGGCACCGAGUCGGUGCUUUU
ACCCCAGUGCUGUUUUCAGUGUUUUAGAGCUAGAAAUAGCAAGUUAAAAUAAGGCUAGUCCGU
UAUCAACUUGAAAAAGUGGCACCGAGUCGGUGCUUUU
CAACAGAAUAUUAUCCAAAUGUUUUAGAGCUAGAAAUAGCAAGUUAAAAUAAGGCUAGUCCGU
UAUCAACUUGAAAAAGUGGCACCGAGUCGGUGCUUUU
UGACACCACAUUGGCAUCGGGUUUUAGAGCUAGAAAUAGCAAGUUAAAAUAAGGCUAGUCCGU
UAUCAACUUGAAAAAGUGGCACCGAGUCGGUGCUUUU
CCACGUUUCAGCUUCUAAGUGUUUUAGAGCUAGAAAUAGCAAGUUAAAAUAAGGCUAGUCCGU
UAUCAACUUGAAAAAGUGGCACCGAGUCGGUGCUUUU
UGAACAAGGUAAGGAGCCUGGUUUUAGAGCUAGAAAUAGCAAGUUAAAAUAAGGCUAGUCCGU
UAUCAACUUGAAAAAGUGGCACCGAGUCGGUGCUUUU
CCACGUUUCAGCUUCUAAGUGUUUUAGAGCUAGAAAUAGCAAGUUAAAAUAAGGCUAGUCCGU
UAUCAACUUGAAAAAGUGGCACCGAGUCGGUGCUUUU
UGACACCACACCAGCAUAGUGUUUUAGAGCUAGAAAUAGCAAGUUAAAAUAAGGCUAGUCCGU
UAUCAACUUGAAAAAGUGGCACCGAGUCGGUGCUUUU
ACCCCAGUGCUGUUUUCAGUGUUUUAGAGCUAGAAAUAGCAAGUUAAAAUAAGGCUAGUCCGU
UAUCAACUUGAAAAAGUGGCACCGAGUCGGUGCUUUU
UGACACCACACCAGCAUAGUGUUUUAGAGCUAGAAAUAGCAAGUUAAAAUAAGGCUAGUCCGU
UAUCAACUUGAAAAAGUGGCACCGAGUCGGUGCUUUU
GUUGGUGCUGAAAUUCAAAGGUUUUAGAGCUAGAAAUAGCAAGUUAAAAUAAGGCUAGUCCGU
UAUCAACUUGAAAAAGUGGCACCGAGUCGGUGCUUUU
UGACACCACAUCAACAUAAUGUUUUAGAGCUAGAAAUAGCAAGUUAAAAUAAGGCUAGUCCGU
UAUCAACUUGAAAAAGUGGCACCGAGUCGGUGCUUUU
CAUGGAUCCCAAUGUCAGAUGUUUUAGAGCUAGAAAUAGCAAGUUAAAAUAAGGCUAGUCCGU
UAUCAACUUGAAAAAGUGGCACCGAGUCGGUGCUUUU
AGGACACUUGAUUCUGUCACGUUUUAGAGCUAGAAAUAGCAAGUUAAAAUAAGGCUAGUCCGU
UAUCAACUUGAAAAAGUGGCACCGAGUCGGUGCUUUU
ACGUACUCCACCACUGUCACGUUUUAGAGCUAGAAAUAGCAAGUUAAAAUAAGGCUAGUCCGU
UAUCAACUUGAAAAAGUGGCACCGAGUCGGUGCUUUU
GUAGCAGUCCUGUACCCCGGGUUUUAGAGCUAGAAAUAGCAAGUUAAAAUAAGGCUAGUCCGU
UAUCAACUUGAAAAAGUGGCACCGAGUCGGUGCUUUU
AGUAGCAGUCCUGUACCCCGGUUUUAGAGCUAGAAAUAGCAAGUUAAAAUAAGGCUAGUCCGU
UAUCAACUUGAAAAAGUGGCACCGAGUCGGUGCUUUU
UAGUAGCAGUCCUGUACCCCGUUUUAGAGCUAGAAAUAGCAAGUUAAAAUAAGGCUAGUCCGU
UAUCAACUUGAAAAAGUGGCACCGAGUCGGUGCUUUU

While some of the guide nucleic acids described herein have been experimentally tested and are known to effect the desired edits, multiple other guide nucleic acid sequences may be compatible with installation of the desired edits. The guide oligonucleotides that have been experimentally tested contained modified nucleotides. Some examples of sequences of modified guide oligonucleotides are provided in Table 4, some of which have been experimentally tested as described in the Examples section below. It will be understood that guide oligonucleotides modified in other manners may also be suitable for effecting the desired edits. For example, the guide oligonucleotides listed in Table 3 may be modified in a manner similar to those listed in Table 4 or in any other suitable manner.

In some embodiments, a guide oligonucleotide comprises one of more modified nucleotides. The nucleotides may be modified in any suitable manner. In some embodiments, the nucleotide comprises a modified sugar moiety. The sugar moiety may be modified in any suitable manner. In embodiments, the modified sugar moieties comprise a 2′-O-methyl, 2′-O-methoxyethyl, 2′-O-aminoethyl, 2′-Fluoro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, or bicyclic modified sugars. In some embodiments, the modified nucleotide comprises a 2′-O-methyl modification. In some embodiments, the nucleotide comprises a backbone modification. In some embodiments, the backbone modifications comprise a phosphorothioate, a phosphorodithioate, a phosphoroselenoate, a phosphorodiselenoate, a phosphoroanilothioate, a phosphoraniladate, a phosphoramidate, or a phosphorodiamidate linkage. In some embodiments, the backbone modification comprises a phosphorothioate linkage.

In some embodiments, a spacer sequence of a guide oligonucleotide comprises from 1 to 20 modified nucleotides. In some embodiments, a spacer sequence comprises from 1 to 10 modified nucleotides. In some embodiments, a spacer sequence comprises from 1 to 5 modified nucleotides. In some embodiments, a spacer sequence comprises from 1 to 3 modified nucleotides. In some embodiments, one or more of the five 5′-most nucleotides of a spacer sequence are modified. In some embodiments, one or more of the five 5′-most nucleotides of a spacer sequence are modified to include a 2′-O-methyl group, a phosphorothioate linkage, or a combination thereof. In some embodiments, one or more of the three 5′-most nucleotides of a spacer sequence are modified. In some embodiments, one or more of the three 5′-most nucleotides of a spacer sequence are modified to include a 2′-O-methyl group, a phosphorothioate linkage, or a combination thereof. In some embodiments, the three 5′-most nucleotides of a spacer sequence are modified. In some embodiments, the three 5′-most nucleotides of a spacer sequence are modified to include a 2′-O-methyl group, a phosphorothioate linkage, or a combination thereof. In some embodiments, the three 5′-most nucleotides of a spacer sequence are modified to include a 2′-O-methyl group and a phosphorothioate linkage.

In some embodiments, a scaffold sequence of a guide oligonucleotide comprises from 1 to 76 modified nucleotides, such as from 1 to 70 modified nucleotides, from 1 to 60 modified nucleotides, or from 1 to 55 modified nucleotides. In some embodiments, a scaffold sequence of a guide oligonucleotide comprises from 10 to 76 modified nucleotides, such as from 20 to 76 modified nucleotides, such as 30 to 76 modified nucleotides, 40 to 76 modified nucleotides, or 50 to 76 modified nucleotides. In some embodiments, a scaffold sequence of a guide oligonucleotide comprises from 20 to 70 modified nucleotides, such as from 30 to 65 modified nucleotides, from 40 to 60 modified nucleotides, or from 50 to 55 modified nucleotides.

In some embodiments, from 20% to 95% of the nucleotides of the scaffold sequence are modified. In some embodiments, from 30% to 90%, such as 40% to 85%, 45% to 80%, 50% to 75%, 60% to 75%, or 65% to 75% of the nucleotides of the scaffold sequence are modified.

In some embodiments, a modified scaffold nucleotide comprises a 2′ sugar modification. In some embodiments, a modified scaffold nucleotide comprises a 2′-O-methyl group. In some embodiments, from 20% to 95% of the nucleotides of the scaffold sequence comprise a 2′ sugar modification. In some embodiments, from 30% to 90%, such as 40% to 85%, 45% to 80%, 50% to 75%, 60% to 75%, or 65% to 75% of the nucleotides of the scaffold sequence comprise a 2′ sugar modification. In some embodiments, from 20% to 95% of the nucleotides of the scaffold sequence comprises a 2′-O-methyl group. In some embodiments, from 30% to 90%, such as 40% to 85%, 45% to 80%, 50% to 75%, 60% to 75%, or 65% to 75% of the nucleotides of the scaffold sequence comprise a 2′-O-methyl group.

Table 5 summarizes the percentages of LPA alleles edited in primary human hepatocytes (editing %, also referred to as editing efficiency) versus selected guide RNA pairs (first and second gRNAs) at relatively higher and lower doses, summarizing the LPA editing efficiencies in primary human hepatocytes using a gene editing system that comprises a Cas9 nickase with the specified pairs of first and second guide RNAs.

Table 6 shows the number of nucleotides between the nicks (the overhang length) for some of the gRNA pairs indicated in Table 5 and indicates the targeted exon. All nicking in Table 5 was in a PAM-out configuration.

In some embodiments, the location of the first nick and the location of the second nick are spaced apart from about 1 to 200 nucleotides, such as from 10 to 150 nucleotides, 15 to 100 nucleotides, or 20 to 50 nucleotides. In some embodiments, the distance between the first and second nicks is 10 nucleotides or greater, such as 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or greater. In some embodiments, the distance between nicks is 200 nucleotides or less, such as 190, 180, 170, 160, 150, 140, 130, 120, 110, 100, 90, 80, 70, 60, or 50 nucleotides or less. In some embodiments, the distance between nicks is from 20 to 50 nucleotides, such as 23 to 45, 30 to 40, 31 to 40, 32 to 40, 33 to 40, 31 to 39, 32 to 39, 33 to 39, or 34 to 38 nucleotides.

Table 4 provides the full gRNA sequences of the guides that were tested to produce the results presented in Table 5.

TABLE 4
Examples of end-modified gRNAs and corresponding further modified gRNAs (having same spacer sequence as end-
modified gRNAs)

| ID | End Modified Sequence | ID | Corresponding Further Modified Sequence |
|---|---|---|---|
| GA1074 | mC*mU*mG*UCACCAGGCAUUGUGUCGUUUUAGAGCUA | GA1438 | mC*mU*mG*UCACCAGGCAUUGUGUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1183 | mG*mU*mA*GUAGCAGUCCUGUACCCGUUUUAGAGCUA | GA1295 | mG*mU*mA*GUAGCAGUCCUGUACCCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1400 | mA*mG*mG*ACACUCGAUUCUGUCACGUUUUAGAGCUA | GA1439 | mA*mG*mG*ACACUCGAUUCUGUCACmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1189 | mC*mU*mG*UCACUGGACAUUGUGUCGUUUUAGAGCUA | GA1301 | mC*mU*mG*UCACUGGACAUUGUGUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA819 | mG*mG*mA*GCAAAGCCCCACAGUCCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1403 | mG*mG*mA*GCAAAGCCCCGGGGUCCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1227 | mC*mU*mU*GGAACUGGGACCACCGUGUUUUAGAGCUA | GA1299 | mC*mU*mU*GGAACUGGGACCACCGUmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1401 | mA*mU*mG*CCAGUGUGGUGUCAUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1225 | mG*mG*mA*GCCAGAAUAACAUUCGGGUUUUAGAGCUA | GA1440 | mG*mG*mA*GCCAGAAUAACAUUCGGmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1231 | mC*mA*mG*AUGCUGAGAUUAGUCCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1383 | mU*mG*mA*CACCACAUUGGCAUCGGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1217 | mC*mA*mU*AGAUGACCAAGAUUGACGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1219 | mC*mC*mA*UCACUGGACAUUGCGUCGUUUUAGAGCUA | GA1441 | mC*mC*mA*UCACUGGACAUUGCGUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1381 | mC*mU*mG*CAUCUGAGCAUCGUGUCGUUUUAGAGCUA | GA1442 | mC*mU*mG*CAUCUGAGCAUCGUGUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1187 | mA*mA*mA*CAGCCGUGGACGUCGCAGUUUUAGAGCUA | GA1443 | mA*mA*mA*CAGCCGUGGACGUCGCAmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1392 | mA*mC*mA*GAGGCUCCUUCUGAACAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1401 | mA*mU*mG*CCAGUGUGGUGUCAUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1387 | mC*mU*mA*UGACACCACAUUGGCAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1072 | mA*mA*mU*AACAUUCGGAGGGACGAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1406 | mG*mA*mA*CAACAGUGGGAGUCUCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1389 | mA*mA*mG*CCUCUGUGCUUGGAUCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1230 | mC*mU*mA*GGGCCUCCUUCUGAACAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1408 | mU*mG*mU*UGUUCCAGUUCCAAGCAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1228 | mC*mU*mU*GGAACCGGGGCCACUGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1216 | mA*mC*mA*UACUUCAUUACUGUCACGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1234 | mU*mA*mC*CCCGGUUCCAAGCCUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1220 | mC*mA*mG*GAAGGACCUGCCAAGCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1395 | mA*mC*mA*ACAGAAUAUUAUCCAAAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1185 | mG*mG*mA*CCACCGUGAGAGUUGCAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1222 | mA*mG*mG*AGAGUUGAUUCCAUCACGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1223 | mG*mU*mG*GUUCUCUGAUGCCAGUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1402 | mA*mC*mC*ACAGAAUACUACCCAAAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1398 | mA*mC*mA*GAGGCUUCUUCUGAAGAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1232 | mU*mA*mG*UCCUUGGUGUUAUACCAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1391 | mC*mA*mC*CAACGGAGCAAAGCCCCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1390 | mG*mU*mU*GUCCUCUGAUGCCAGUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1226 | mU*mG*mA*CACCACAUCAACAUAAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1394 | mC*mG*mG*AUCGGUUGUGUAACACCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1233 | mU*mC*mG*CCCUUGGUGUUACACCAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1221 | mC*mA*mG*GAAGAACCUGCCAAGCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1224 | mG*mG*mU*AAUAGUUGGAGGCGCGAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1191 | mA*mC*mU*GUGGGGCUUUGCUCCGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1396 | mC*mU*mG*UGUCUGAGCAUCGCGUCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1407 | mC*mA*mU*GGAUCCCAGUGUCAGAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1399 | mG*mC*mA*CCAACGGAGCAAAGCCCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1393 | mU*mG*mA*AAACAGCACUGGGGUCCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1384 | mA*mA*mC*UCAAGGUUGUACCCAUUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1380 | mG*mG*mG*AUCCUCCGAUGCCAAUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1382 | mC*mU*mA*CUUAGAAGCUGAAACGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA810 | mG*mG*mC*UCCUUACCUUGUUCAGAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1385 | mC*mC*mU*ACUUAGAAGCUGAAACGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1405 | mG*mG*mG*GUCCGACUAUGCUGGUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1388 | mC*mA*mC*CAACUGAAAACAGCACUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1404 | mU*mU*mU*CUGGGGUCCGACUAUGCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1409 | mU*mC*mU*UUGAAUUUCAGCACCAAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1386 | mG*mU*mG*GUCCUAUUAUGUUGAUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1397 | mC*mC*mA*UCUGACAUUGGGAUCCAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1229 | mU*mC*mC*ACGGCUGUUUCUGAACAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1076 | mU*mG*mG*ACCACAUGGCUUUGCUCGUUUUAGAGCUA | GA1303 | mU*mG*mG*ACCACAUGGCUUUGCUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1075 | mU*mG*mU*CCUUGCAACUCUCACGGGUUUUAGAGCUA | GA1444 | mU*mG*mU*CCUUGCAACUCUCACGGmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1184 | mC*mA*mU*UAUGGACAGAGUUACCGGUUUUAGAGCUA | GA1296 | mC*mA*mU*UAUGGACAGAGUUACCGmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1423 | mC*mA*mC*AACUCCCACAGUGGCCCGUUUUAGAGCUA | GA1445 | mC*mA*mC*AACUCCCACAGUGGCCCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1190 | mA*mA*mG*UGUCCUUGCGACGUCCAGUUUUAGAGCUA | GA1302 | mA*mA*mG*UGUCCUUGCGACGUCCAmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1418 | mG*mU*mU*GGUGCUGAAAUUCAAAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1418 | mG*mU*mU*GGUGCUGAAAUUCAAAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1245 | mA*mC*mA*GAGCUUCCUUCUGAAGAGUUUUAGAGCUA | GA1300 | mA*mC*mA*GAGCUUCCUUCUGAAGAmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1402 | mA*mC*mC*ACAGAAUACUACCCAAAGUUUUAGAGCUA | | mA*mC*mC*ACAGAAUACUACCCAAAmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1244 | mC*mU*mA*GAGGCUUUUUUUGAACAGUUUUAGAGCUA | GA1446 | mC*mU*mA*GAGGCUUUUUUUGAACAmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1250 | mU*mG*mG*AUUCCUGCAGUAGUUCCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1412 | mA*mC*mA*UGUUCUUCCUGUGAUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1236 | mU*mG*mA*UACCACACUGGCAUCAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1238 | mA*mA*mC*UCUCCUCACAACUCCCAGUUUUAGAGCUA | GA1447 | mA*mA*mC*UCUCCUCACAACUCCCAmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1410 | mC*mG*mU*CCCUCCGAAUGUUAUUCGUUUUAGAGCUA | GA1448 | mC*mG*mU*CCCUCCGAAUGUUAUUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1188 | mU*mG*mA*ACAAGGUAAGAAGUCUCGUUUUAGAGCUA | GA1449 | mU*mG*mA*ACAAGGUAAGAAGUCUCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1419 | mG*mC*mU*UGGAACCGGGGCCACUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1395 | mA*mC*mA*ACAGAAUAUUAUCCAAAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1412 | mA*mC*mA*UGUUCUUCCUGUGAUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1073 | mU*mA*mU*UCUGGCUCCAAGCCUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1426 | mU*mG*mU*UCCAGUUCCAAGCAUGGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1416 | mA*mG*mG*CUUCUUCUGAAGAAGGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1249 | mC*mU*mU*GGAACCUGGAUGACAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1406 | mG*mA*mA*CAACAGUGGGAGUCUCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1246 | mA*mC*mA*GAGGCUCCUUCUGAACAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1235 | mG*mA*mU*AACUCUGUCCAUUUCCGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1253 | mG*mG*mU*AACAGUCGGAGGCGCGAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1239 | mG*mG*mU*CCUUCCUGUGACAGUGGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1390 | mG*mU*mU*GUCCUCUGAUGCCAGUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1186 | mG*mG*mU*CCCAGAUCCAAGCACAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1241 | mU*mC*mU*CCUCACAACUCCCACGGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1242 | mC*mC*mA*CAGAAUACUACCCAAAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1424 | mG*mU*mG*GUUCUCUGAUGCCAGUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1389 | mA*mA*mG*CCUCUGUGCUUGGAUCUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1251 | mC*mA*mA*GGACUAAUCUCAGCAUCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1418 | mG*mU*mU*GGUGCUGAAAUUCAAAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1413 | mC*mA*mA*CAGAAUAUUAUCCAAAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1218 | mC*mA*mU*AGAUGACCAAGCUUGGCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1420 | mA*mA*mC*CGAUCCGUGUGUGAGGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1252 | mC*mA*mA*GGGCGAAUCUCAGCAUCGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1240 | mG*mG*mU*UCUUCCUGUGACAGUGGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1243 | mU*mA*mC*CCCGAUUCCAAGCCUAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1192 | mA*mC*mA*GUCCAGGACUGCUACCAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1421 | mC*mU*mC*AGACACAGAAGGGACUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1427 | mG*mA*mU*CCAUGGUAUAACACCAAGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1418 | mG*mU*mU*GGUGCUGAAAUUCAAAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1415 | mA*mC*mC*CCAGUGCUGUUUUCAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1413 | mC*mA*mA*CAGAAUAUUAUCCAAAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1383 | mU*mG*mA*CACCACAUUGGCAUCGGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1411 | mC*mC*mA*CGUUUCAGCUUCUAAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1417 | mU*mG*mA*ACAAGGUAAGGAGCCUGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1411 | mC*mC*mA*CGUUUCAGCUUCUAAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1425 | mU*mG*mA*CACCACACCAGCAUAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1415 | mA*mC*mC*CCAGUGCUGUUUUCAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1425 | mU*mG*mA*CACCACACCAGCAUAGUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1418 | mG*mU*mU*GGUGCUGAAAUUCAAAGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1414 | mU*mG*mA*CACCACAUCAACAUAAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1422 | mC*mA*mU*GGAUCCCAAUGUCAGAUGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1248 | mA*mG*mG*ACACUUGAUUCUGUCACGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1270 | mA*mC*mG*UACUCCACCACUGUCACGUUUUAGAGCUA | GA1304 | mA*mC*mG*UACUCCACCACUGUCACmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1264 | mG*mU*mA*GCAGUCCUGUACCCCGGGUUUUAGAGCUA | GA1297 | mG*mU*mA*GCAGUCCUGUACCCCGGmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |
| GA1265 | mA*mG*mU*AGCAGUCCUGUACCCCGGUUUUAGAGCUA | | |
| | GAAAUAGCAAGUUAAAAUAAGGCUAGUCCGUUAUCAAC | | |
| | UUGAAAAAGUGGCACCGAGUCGGUGCU*mU*mU*mU | | |
| GA1266 | mU*mA*mG*UAGCAGUCCUGUACCCCGUUUUAGAGCUA | GA1298 | mU*mA*mG*UAGCAGUCCUGUACCCCmGUUUUAGmAmGmCmUmAGmAmAmAm |
| | GAAAUAGCAAGUUAAAAUAAGGCUAGUCCGUUAUCAAC | | UmAmGmCmAmAGUUmAAmAAmUAmAmGmGmCmUmAGUmCmCGUUAmUmCAAm |
| | UUGAAAAAGUGGCACCGAGUCGGUGCU*mU*mU*mU | | CmUmUGmAmAmAmAmAmGmUmGGmCmAmCmCmGmAmGmUmCmGmGmUmGmCm |
| | | | U*mU*mU*mU |

m = 2′ O-methyl analog; *= 3′ phosphorothioate; the first 20 nucleotides of each sequence correspond to the spacer sequence; no guide ID indicates that a guide ID has not yet been assigned

TABLE 5
Guide RNA identifiers and editing efficiencies
of selected pairs of guide RNA pairs at different doses

| | | | | | | | Average Editing % | |
|---|---|---|---|---|---|---|---|---|
| Guide 1 | MIT | | Guide 2 | MIT | | | 2500 | 312.5 |
| ID | score | Protospacer | ID | Score | Protospacer | Exon | ng/ml | ng/ml |
| GA1074 | 58 | CTGTCACCAGGCATTGTGTC | GA1075 | 83 | TGTCCTTGCAACTCTCACGG | 21 | 93.775 | 59.445 |
| GA1183 | 78 | GTAGTAGCAGTCCTGTACCC | GA1184 | 82 | CATTATGGACAGAGTTACCG | 20 | 93.34 | 63.31 |
| GA1264 | | GTAGCAGTCCTGTACCCCGG | GA1184 | 82 | CATTATGGACAGAGTTACCG | 20 | * | |
| GA1265 | | AGTAGCAGTCCTGTACCCCG | GA1184 | 82 | CATTATGGACAGAGTTACCG | 20 | ** | |
| GA1266 | | TAGTAGCAGTCCTGTACCCC | GA1184 | 82 | CATTATGGACAGAGTTACCG | 20 | * | |
| GA1400 | 46 | AGGACACTCGATTCTGTCAC | GA1423 | 45 | CACAACTCCCACAGTGGCCC | 27 | 88.7 | 61.445 |
| GA1189 | 58 | CTGTCACTGGACATTGTGTC | GA1190 | 92 | AAGTGTCCTTGCGACGTCCA | 23 | 87.395 | 60.51 |
| GA819 | 53 | GGAGCAAAGCCCCACAGTCC | GA1418 | 34 | GTTGGTGCTGAAATTCAAAG | 24 | 86.01 | 56.36 |
| GA1403 | 46 | GGAGCAAAGCCCCGGGGTCC | GA1418 | 34 | GTTGGTGCTGAAATTCAAAG | 22 | 85.75 | 55.155 |
| GA1227 | 72 | CTTGGAACTGGGACCACCGT | GA1245 | 46 | ACAGAGCTTCCTTCTGAAGA | 25 | 85.625 | 49.525 |
| GA1401 | 42 | ATGCCAGTGTGGTGTCATAG | GA1402 | 38 | ACCACAGAATACTACCCAAA | 24 | 84.47 | 35.57 |
| GA1225 | 87 | GGAGCCAGAATAACATTCGG | GA1244 | 57 | CTAGAGGCTTTTTTTGAACA | 19 | 83.72 | 49.355 |
| GA1231 | 68 | CAGATGCTGAGATTAGTCCT | GA1250 | 13 | TGGATTCCTGCAGTAGTTCC | 23 | 83.36 | 47.22 |
| GA1383 | 82 | TGACACCACATTGGCATCGG | GA1412 | 41 | ACATGTTCTTCCTGTGATAG | 26 | 83.19 | 52.08 |
| GA1217 | 70 | CATAGATGACCAAGATTGAC | GA1236 | 37 | TGATACCACACTGGCATCAG | 28 | 81.115 | 38.825 |
| GA1219 | 86 | CCATCACTGGACATTGCGTC | GA1238 | 60 | AACTCTCCTCACAACTCCCA | 25 | 80.99 | 38.575 |
| GA1381 | 72 | CTGCATCTGAGCATCGTGTC | GA1410 | 84 | CGTCCCTCCGAATGTTATTC | 19 | 80.895 | 38.58 |
| GA1187 | 94 | AAACAGCCGTGGACGTCGCA | GA1188 | 60 | TGAACAAGGTAAGAAGTCTC | 23 | 80.23 | 39.055 |
| GA1392 | 52 | ACAGAGGCTCCTTCTGAACA | GA1419 | 50 | GCTTGGAACCGGGGCCACTG | 27 | 78.77 | 58.075 |
| GA1401 | 42 | ATGCCAGTGTGGTGTCATAG | GA1395 | 47 | ACAACAGAATATTATCCAAA | 22 | 78.33 | 57.31 |
| GA1387 | 67 | CTATGACACCACATTGGCAT | GA1412 | 41 | ACATGTTCTTCCTGTGATAG | 26 | 76.36 | 47.525 |
| GA1072 | 82 | AATAACATTCGGAGGGACGA | GA1073 | 70 | TATTCTGGCTCCAAGCCTAG | 19 | 74.67 | 38.795 |
| GA1406 | 38 | GAACAACAGTGGGAGTCTCT | GA1426 | 37 | TGTTCCAGTTCCAAGCATGG | 29 | 67.475 | 24.695 |
| GA1389 | 57 | AAGCCTCTGTGCTTGGATCT | GA1416 | 55 | AGGCTTCTTCTGAAGAAGGT | 21 | 63.775 | 29.81 |
| GA1230 | 64 | CTAGGGCCTCCTTCTGAACA | GA1249 | 51 | CTTGGAACCTGGATGACAGT | 31 | 62.355 | 42.215 |
| GA1408 | 37 | TGTTGTTCCAGTTCCAAGCA | GA1406 | 38 | GAACAACAGTGGGAGTCTCT | 29 | 57.88 | 18.605 |
| GA1228 | 62 | CTTGGAACCGGGGCCACTGT | GA1246 | 52 | ACAGAGGCTCCTTCTGAACA | 27 | 56.11 | 42.75 |
| GA1216 | 66 | ACATACTTCATTACTGTCAC | GA1235 | 29 | GATAACTCTGTCCATTTCCG | 18 | 55.825 | 41.415 |
| GA1234 | 11 | TACCCCGGTTCCAAGCCTAG | GA1253 | 14 | GGTAACAGTCGGAGGCGCGA | 3 | 55.42 | 22.83 |
| GA1220 | 9 | CAGGAAGGACCTGCCAAGCT | GA1239 | 8 | GGTCCTTCCTGTGACAGTGG | 2 | 53.19 | 18.185 |
| GA1395 | 47 | ACAACAGAATATTATCCAAA | GA1390 | 56 | GTTGTCCTCTGATGCCAGTG | 22 | 52.805 | 15.505 |
| GA1185 | 68 | GGACCACCGTGAGAGTTGCA | GA1186 | 53 | GGTCCCAGATCCAAGCACAG | 21 | 51.865 | 13.155 |
| GA1222 | 64 | AGGAGAGTTGATTCCATCAC | GA1241 | 70 | TCTCCTCACAACTCCCACGG | 25 | 51.45 | 36.005 |
| GA1223 | 51 | GTGGTTCTCTGATGCCAGTG | GA1242 | 41 | CCACAGAATACTACCCAAAT | 24 | 51.18 | 33.96 |
| GA1402 | 38 | ACCACAGAATACTACCCAAA | GA1424 | 51 | GTGGTTCTCTGATGCCAGTG | 24 | 45.395 | 16.705 |
| GA1398 | 46 | ACAGAGGCTTCTTCTGAAGA | GA1389 | 57 | AAGCCTCTGTGCTTGGATCT | 21 | 44.575 | 14.34 |
| GA1232 | 68 | TAGTCCTTGGTGTTATACCA | GA1251 | 59 | CAAGGACTAATCTCAGCATC | 23 | 43.135 | 6.135 |
| GA1391 | 60 | CACCAACGGAGCAAAGCCCC | GA1418 | 34 | GTTGGTGCTGAAATTCAAAG | 22 | 40.525 | 7.865 |
| GA1390 | 56 | GTTGTCCTCTGATGCCAGTG | GA1413 | 52 | CAACAGAATATTATCCAAAT | 22 | 37.98 | 14.16 |
| GA1226 | 69 | TGACACCACATCAACATAAT | GA1218 | 8 | CATAGATGACCAAGCTTGGC | 2 | 36.43 | 11.235 |
| GA1394 | 56 | CGGATCGGTTGTGTAACACC | GA1420 | 40 | AACCGATCCGTGTGTGAGGT | 29 | 34.485 | 6.575 |
| GA1233 | 41 | TCGCCCTTGGTGTTACACCA | GA1252 | 23 | CAAGGGCGAATCTCAGCATC | 21 | 33.12 | 7.845 |
| GA1221 | 7 | CAGGAAGAACCTGCCAAGCT | GA1240 | 9 | GGTTCTTCCTGTGACAGTGG | 10 | 32.885 | 7.295 |
| GA1224 | 75 | GGTAATAGTTGGAGGCGCGA | GA1243 | 12 | TACCCCGATTCCAAGCCTAG | 17 | 27.755 | 12.62 |
| GA1191 | 80 | ACTGTGGGGCTTTGCTCCGT | GA1192 | 62 | ACAGTCCAGGACTGCTACCA | 23 | 27.195 | 8.325 |
| GA1396 | 58 | CTGTGTCTGAGCATCGCGTC | GA1421 | 33 | CTCAGACACAGAAGGGACTG | 31 | 24.16 | 2.955 |
| GA1407 | 38 | CATGGATCCCAGTGTCAGAT | GA1427 | 36 | GATCCATGGTATAACACCAA | 25 | 20.875 | 7.325 |
| GA1399 | 57 | GCACCAACGGAGCAAAGCCC | GA1418 | 34 | GTTGGTGCTGAAATTCAAAG | 22 | 20.185 | 2.71 |
| GA1393 | 47 | TGAAAACAGCACTGGGGTCC | GA1415 | 60 | ACCCCAGTGCTGTTTTCAGT | 26 | 6.86 | 0.62 |
| GA1384 | 76 | AACTCAAGGTTGTACCCATT | GA1413 | 52 | CAACAGAATATTATCCAAAT | 22 | 6.185 | 1.765 |
| GA1380 | 74 | GGGATCCTCCGATGCCAATG | GA1383 | 82 | TGACACCACATTGGCATCGG | 26 | 3.92 | 3.41 |
| GA1382 | 81 | CTACTTAGAAGCTGAAACGT | GA1411 | 60 | CCACGTTTCAGCTTCTAAGT | 39 | 3.755 | 0.285 |
| GA810 | 59 | GGCTCCTTACCTTGTTCAGA | GA1417 | 39 | TGAACAAGGTAAGGAGCCTG | 17 | 2.78 | 0.38 |
| GA1385 | 71 | CCTACTTAGAAGCTGAAACG | GA1411 | 60 | CCACGTTTCAGCTTCTAAGT | 39 | 2.165 | 0.365 |
| GA1405 | 35 | GGGGTCCGACTATGCTGGTG | GA1425 | 46 | TGACACCACACCAGCATAGT | 20 | 0.8 | 0.175 |
| GA1388 | 56 | CACCAACTGAAAACAGCACT | GA1415 | 60 | ACCCCAGTGCTGTTTTCAGT | 26 | 0.465 | 0.415 |
| GA1404 | 38 | TTTCTGGGGTCCGACTATGC | GA1425 | 46 | TGACACCACACCAGCATAGT | 20 | 0.33 | 0.775 |
| GA1409 | 37 | TCTTTGAATTTCAGCACCAA | GA1418 | 34 | GTTGGTGCTGAAATTCAAAG | 22 | 0.31 | 0.17 |
| GA1386 | 64 | GTGGTCCTATTATGTTGATG | GA1414 | 69 | TGACACCACATCAACATAAT | 2 | 0.055 | 0.065 |
| GA1397 | 48 | CCATCTGACATTGGGATCCA | GA1422 | 53 | CATGGATCCCAATGTCAGAT | 23 | 0.055 | 0.04 |
| GA1229 | 65 | TCCACGGCTGTTTCTGAACA | GA1248 | 40 | AGGACACTTGATTCTGTCAC | 21 | | |
| GA1076 | 59 | TGGACCACATGGCTTTGCTC | GA1076 | 10 | ACGTACTCCACCACTGTCAC | 2 | | |

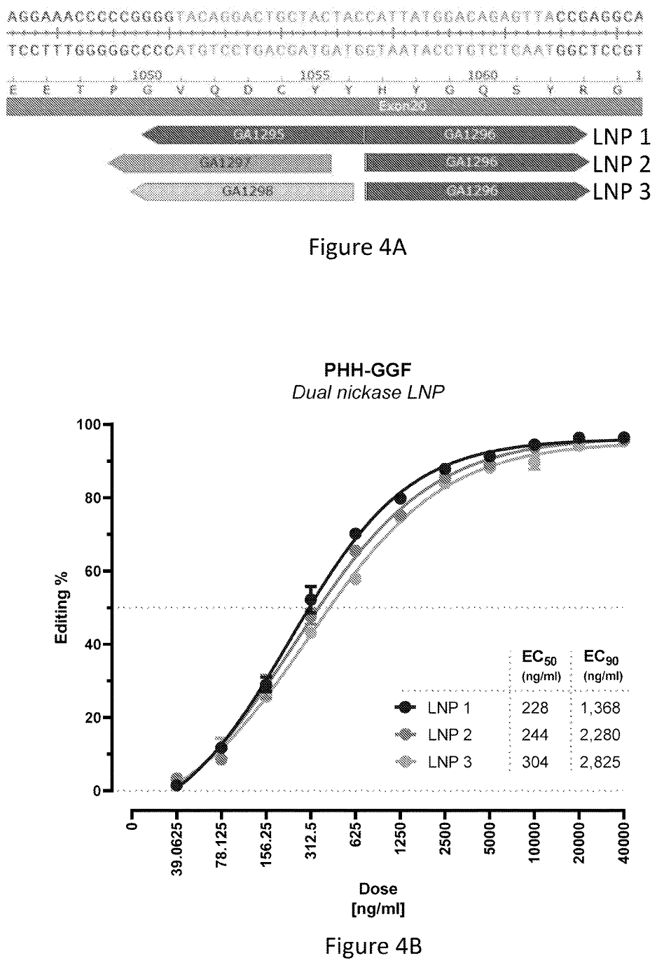

The tested guide oligonucleotides comprise RNA spacer sequences identical to the protospacer sequences shown in the table above, but with “U” substituted for “T”. Editing results are presented for those gRNA pairs that were evaluated by MessengerMax transfection.
* Editing results for in vitro LNP delivery studies for guide pairs having spacer sequences corresponding to GA1264/GA1184 (GA1297/GA1296) and GA1266/GA1184 (GA1298/GA1296) are shown in FIG. 4B and were similar to a guide pair having spacer sequences corresponding to GA1183/GA1184 (GA1295/GA1296) - see, also, Table 4 and Table 7B.
** The spacer for guide GA1265 overlaps with the spacers for GA1264 (and GA1297), GA1266 (and GA1298), and GA1184 (and GA1295) (see FIG. 4A for alignment of spacer/protospacer sequences of GA1297, GA1298, and GA1295) the protospacer for GA1265 is between (and offset by one from) protospacer for GA1264 (and GA1297) and GA1266 (and GA1298).

TABLE 6
Spacing between nicks and target exons for selected gRNA pairs

| Guide RNA Pair ID | Nucleotides between nicks | Target Exon |
|---|---|---|
| GA1183/GA1184 | 34 | 20 |
| GA1227/GA1245 | 35 | 25 |
| GA1187/GA1188 | 35 | 23 |
| GA1230/GA1249 | 35 | 31 |
| GA1189/GA1190 | 38 | 23 |
| GA1220/GA1239 | 23 | 2 |
| GA1225/GA1244 | 38 | 19 |
| GA1224/GA1243 | 30 | 17 |
| GA1231/GA1250 | 32 | 23 |
| GA1223/GA1242 | 30 | 24 |
| GA1219/GA1238 | 38 | 25 |
| GA1228/GA1246 | 35 | 27 |
| GA1217/GA1236 | 32 | 28 |
| GA1072/GA1073 | 30 | 19 |
| GA1074/GA1075 | 41 | 21 |
| GA1216/GA1235 | 39 | 18 |
| GA1222/GA1241 | 28 | 25 |
| GA1229/GA1248 | 41 | 23 |
| GA1185/GA1186 | 29 | 21 |
| GA1234/GA1253 | 30 | 3 |
| GA1191/GA1192 | 29 | 23 |
| GA1232/GA1251 | 25 | 23 |
| GA1233/GA1252 | 25 | 21 |
| GA1400/GA1423 | 34 | 27 |
| GA819/GA1418 | 34 | 24 |
| GA1403/GA1418 | 34 | 22 |
| GA1401/GA1402 | 40 | 24 |
| GA1383/GA1412 | 50 | 26 |
| GA1381/GA1410 | 47 | 19 |
| GA1392/GA1419 | 34 | 27 |
| GA1401/GA1395 | 40 | 22 |
| GA1387/GA1412 | 47 | 26 |
| GA1406/GA1426 | 29 | 29 |
| GA1389/GA1416 | 28 | 21 |
| GA1408/GA1406 | 26 | 29 |
| GA1395/GA1390 | 29 | 27 |
| GA1402/GA1424 | 29 | 24 |
| GA1398/GA1389 | 24 | 21 |
| GA1391/GA1418 | 27 | 22 |
| GA1390/GA1413 | 30 | 22 |
| GA1226/GA1218 | 32 | 2 |
| GA1394/GA1420 | 24 | 29 |
| GA1221/GA1240 | 23 | 4 |
| GA1396/GA1421 | 23 | 31 |
| GA1407/GA1427 | 26 | 25 |
| GA1399/GA1418 | 26 | 22 |
| GA1393/GA1415 | 16 | 26 |
| GA1384/GA1413 | 3 | 22 |
| GA1380/GA1383 | 6 | 26 |
| GA1382/GA1411 | 12 | 39 |
| GA810/GA1417 | 16 | 17 |
| GA1385/GA1411 | 11 | 39 |
| GA1405/GA1425 | 6 | 20 |
| GA1388/GA1415 | 9 | 26 |
| GA1404/GA1425 | 1 | 20 |
| GA1409/GA1418 | 13 | 22 |
| GA1386/GA1414 | 6 | 2 |
| GA1397/GA1422 | 12 | 23 |

In embodiments, a gene editing system described herein comprises a first guide oligonucleotide and a second guide oligonucleotide, where the first guide oligonucleotide comprises a spacer sequence identical to or substantially identical to a guide 1 protospacer sequence specified in Table 5, and the second guide oligonucleotide comprises a spacer sequence identical to or substantially identical to a corresponding (in the same row) guide 2 protospacer sequence specified in Table 5. In embodiments, the first guide oligonucleotide comprises a spacer sequence identical to or substantially identical to a guide 1 protospacer sequence specified in Table 5, and the second guide oligonucleotide comprises a spacer sequence identical to or substantially identical to a corresponding guide 2 protospacer sequence specified in Table 5, wherein the tested guide pair of Table 5 exhibited an editing of 20% or greater, of 30% or greater, of 40% or greater, of 50% or greater, of 60% or greater, of 70% or greater, of 80% or greater, of 85% or greater, or of 90% or greater at a concentration of 2500 ng/ml, or where the gRNA pair corresponds to GA1264/GA1184, GA1265/GA1184, or GA1266/GA1184. In embodiments, the gene editing system comprises a first guide oligonucleotide and a second guide oligonucleotide, where the first guide oligonucleotide comprises a spacer sequence identical to or substantially identical to a guide 1 protospacer sequence specified in Table 5, and the second guide oligonucleotide comprises a spacer sequence identical to or substantially identical to a corresponding guide 2 protospacer sequence specified in Table 5 wherein the tested guide pair of Table 5 exhibited an editing of 50% or greater, of 60% or greater, of 70% or greater, of 80% or greater, of 85% or greater, or of 90% or greater at a concentration of 2500 ng/ml, or where the gRNA pair corresponds to GA1264/GA1184, GA1265/GA1184, or GA1266/GA1184. In embodiments, the first guide oligonucleotide comprises a spacer sequence identical to or substantially identical to a guide 1 protospacer sequence specified in Table 5, and the second guide oligonucleotide comprises a spacer sequence identical to or substantially identical to a corresponding guide 2 protospacer sequence specified in Table 5, wherein the tested guide pair exhibited an editing of 70% or greater, of 80% or greater, of 85% or greater, or of 90% or greater at a concentration of 2500 ng/ml, or where the guide pair corresponds to GA1264/GA1184, GA1265/GA1184, or GA1266/GA 1184.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CUGUCACCAGGCAUUGUGUC-3′; and the second guide oligonucleotide comprises a spacer sequence comprising nucleotides 6 to 20, 5 to 20, 4 to 20, 3 to 20, 2 to 20, or 1 to 20 of the following sequence 5′-UGUCCUUGCAACUCUCACGG-3′. For clarity, an oligonucleotide comprising nucleotides 6 to 20 of 5′-CUGUCACCAGGCAUUGUGUC-3′ would comprise 5′-ACCAGGCAUUGUGUC-3′. In some embodiments, the first guide oligonucleotide comprises a spacer having a sequence that is at least about 75%, at least about 80%, at least about 85%, at least about 90%, at least about 95% identical to, or is identical to 5′-CUGUCACCAGGCAUUGUGUC-3′, and the second guide oligonucleotide comprises a spacer having a sequence that is at least about 75%, at least about 80%, at least about 85%, at least about 90%, at least about 95% identical to, or is identical to 5′-UGUCCUUGCAACUCUCACGG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-GUAGUAGCAGUCCUGUACCC-3′; and the second guide oligonucleotide comprises a spacer sequence comprising nucleotides 6 to 20, 5 to 20, 4 to 20, 3 to 20, 2 to 20, or 1 to 20 of the following sequence 5′-CAUUAUGGACAGAGUUACCG-3′. In some embodiments, the first guide oligonucleotide comprises a spacer having a sequence that is at least about 75%, at least about 80%, at least about 85%, at least about 90%, at least about 95% identical to, or is identical to 5′-GUAGUAGCAGUCCUGUACCC-3′, and the second guide oligonucleotide comprises a spacer having a sequence that is at least about 75%, at least about 80%, at least about 85%, at least about 90%, at least about 95% identical to, or is identical to 5′-CAUUAUGGACAGAGUUACCG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-AGGACACUCGAUUCUGUCAC-3′; and the second guide oligonucleotide comprises a spacer sequence comprising nucleotides 6 to 20, 5 to 20, 4 to 20, 3 to 20, 2 to 20, or 1 to 20 of the following sequence 5′-CACAACUCCCACAGUGGCCC-3′. In some embodiments, the first guide oligonucleotide comprises a spacer having a sequence that is at least about 75%, at least about 80%, at least about 85%, at least about 90%, at least about 95% identical to, or is identical to 5′-AGGACACUCGAUUCUGUCA-3′, and the second guide oligonucleotide comprises a spacer having a sequence that is at least about 75%, at least about 80%, at least about 85%, at least about 90%, at least about 95% identical to, or is identical to 5′-CACAACUCCCACAGUGGCCC-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CUGUCACUGGACAUUGUGUC-3′; and the second guide oligonucleotide comprises a spacer sequence comprising nucleotides 6 to 20, 5 to 20, 4 to 20, 3 to 20, 2 to 20, or 1 to 20 of the following sequence 5′-AAGUGUCCUUGCGACGUCCA-3′. In some embodiments, the first guide oligonucleotide comprises a spacer having a sequence that is at least about 75%, at least about 80%, at least about 85%, at least about 90%, at least about 95% identical to, or is identical to 5′-CUGUCACUGGACAUUGUGUC-3′, and the second guide oligonucleotide comprises a spacer having a sequence that is at least about 75%, at least about 80%, at least about 85%, at least about 90%, at least about 95% identical to, or is identical to 5′-AAGUGUCCUUGCGACGUCCA-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-GGAGCAAAGCCCCACAGUCC-3′; and the second guide oligonucleotide comprises a spacer sequence comprising nucleotides 6 to 20, 5 to 20, 4 to 20, 3 to 20, 2 to 20, or 1 to 20 of the following sequence 5′-GUUGGUGCUGAAAUUCAAAG-3′. In some embodiments, the first guide oligonucleotide comprises a spacer having a sequence that is at least about 75%, at least about 80%, at least about 85%, at least about 90%, at least about 95% identical to, or is identical to 5′-GGAGCAAAGCCCCACAGUCC-3′, and the second guide oligonucleotide comprises a spacer having a sequence that is at least about 75%, at least about 80%, at least about 85%, at least about 90%, at least about 95% identical to, or is identical to 5′-GUUGGUGCUGAAAUUCAAAG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-GGAGCAAAGCCCCGGGGUCC-3′; and the second guide oligonucleotide comprises a spacer sequence comprising nucleotides 6 to 20, 5 to 20, 4 to 20, 3 to 20, 2 to 20, or 1 to 20 of the following sequence 5′-GUUGGUGCUGAAAUUCAAAG-3′. In some embodiments, the first guide oligonucleotide comprises a spacer having a sequence that is at least about 75%, at least about 80%, at least about 85%, at least about 90%, at least about 95% identical to, or is identical to 5′-GGAGCAAAGCCCCGGGGUCC-3′, and the second guide oligonucleotide comprises a spacer having a sequence that is at least about 75%, at least about 80%, at least about 85%, at least about 90%, at least about 95% identical to, or is identical to 5′-GUUGGUGCUGAAAUUCAAAG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CUGGAACUGGGACCACCGU-3′; and the second guide oligonucleotide comprises a spacer sequence comprising nucleotides 6 to 20, 5 to 20, 4 to 20, 3 to 20, 2 to 20, or 1 to 20 of the following sequence 5′-ACAGAGCUUCCUUCUGAAGA-3′. In some embodiments, the first guide oligonucleotide comprises a spacer having a sequence that is at least about 75%, at least about 80%, at least about 85%, at least about 90%, at least about 95% identical to, or is identical to 5′-CUGGAACUGGGACCACCGU-3′, and the second guide oligonucleotide comprises a spacer having a sequence that is at least about 75%, at least about 80%, at least about 85%, at least about 90%, at least about 95% identical to, or is identical to 5′-ACAGAGCUUCCUUCUGAAGA-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-AUGCCAGUGUGGUGUCAUAG-3′; and the second guide oligonucleotide comprises a spacer sequence comprising nucleotides 6 to 20, 5 to 20, 4 to 20, 3 to 20, 2 to 20, or 1 to 20 of the following sequence 5′-ACCACAGAAUACUACCCAAA-3′. In some embodiments, the first guide oligonucleotide comprises a spacer having a sequence that is at least about 75%, at least about 80%, at least about 85%, at least about 90%, at least about 95% identical to, or is identical to 5′-AUGCCAGUGUGGUGUCAUAG-3′, and the second guide oligonucleotide comprises a spacer having a sequence that is at least about 75%, at least about 80%, at least about 85%, at least about 90%, at least about 95% identical to, or is identical to 5′-ACCACAGAAUACUACCCAAA-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-GGAGCCAGAAUAACAUUCGG-3′; and the second guide oligonucleotide comprises a spacer sequence comprising nucleotides 6 to 20, 5 to 20, 4 to 20, 3 to 20, 2 to 20, or 1 to 20 of the following sequence 5′-CUAGAGGCUUUUIUUUGAACA-3′. In some embodiments, the first guide oligonucleotide comprises a spacer having a sequence that is at least about 75%, at least about 80%, at least about 85%, at least about 90%, at least about 95% identical to, or is identical to 5′-GGAGCCAGAAUAACAUUCGG-3′, and the second guide oligonucleotide comprises a spacer having a sequence that is at least about 75%, at least about 80%, at least about 85%, at least about 90%, at least about 95% identical to, or is identical to 5′-CUAGAGGCUUUUUUUGAACA-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CAGAUGCUGAGAUUAGUCCU-3′; and the second guide oligonucleotide comprises a spacer sequence comprising nucleotides 6 to 20, 5 to 20, 4 to 20, 3 to 20, 2 to 20, or 1 to 20 of the following sequence 5′-UGGAUUCCUGCAGUAGUUCC-3′. In some embodiments, the first guide oligonucleotide comprises a spacer having a sequence that is at least about 75%, at least about 80%, at least about 85%, at least about 90%, at least about 95% identical to, or is identical to 5′-CAGAUGCUGAGAUUAGUCCU-3′, and the second guide oligonucleotide comprises a spacer having a sequence that is at least about 75%, at least about 80%, at least about 85%, at least about 90%, at least about 95% identical to, or is identical to 5′-UGGAUUCCUGCAGUAGUUCC-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-UGACACCACAUUGGCAUCGG-3′; and the second guide oligonucleotide comprises a spacer sequence comprising nucleotides 6 to 20, 5 to 20, 4 to 20, 3 to 20, 2 to 20, or 1 to 20 of the following sequence 5′-ACAUGUUCUUCCUGUGAUAG-3′. In some embodiments, the first guide oligonucleotide comprises a spacer having a sequence that is at least about 75%, at least about 80%, at least about 85%, at least about 90%, at least about 95% identical to, or is identical to 5′-UGACACCACAUUGGCAUCGG-3′, and the second guide oligonucleotide comprises a spacer having a sequence that is at least about 75%, at least about 80%, at least about 85%, at least about 90%, at least about 95% identical to, or is identical to 5′-ACAUGUUCUUCCUGUGAUAG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CAUAGAUGACCAAGAUUGAC-3′; and the second guide oligonucleotide comprises a spacer sequence comprising nucleotides 6 to 20, 5 to 20, 4 to 20, 3 to 20, 2 to 20, or 1 to 20 of the following sequence 5′-UGAUACCACACUGGCAUCAG-3′. In some embodiments, the first guide oligonucleotide comprises a spacer having a sequence that is at least about 75%, at least about 80%, at least about 85%, at least about 90%, at least about 95% identical to, or is identical to 5′-CAUAGAUGACCAAGAUUGAC-3′, and the second guide oligonucleotide comprises a spacer having a sequence that is at least about 75%, at least about 80%, at least about 85%, at least about 90%, at least about 95% identical to, or is identical to 5′-UGAUACCACACUGGCAUCAG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CCAUCACUGGACAUUGCGUC-3′; and the second guide oligonucleotide comprises a spacer sequence comprising nucleotides 6 to 20, 5 to 20, 4 to 20, 3 to 20, 2 to 20, or 1 to 20 of the following sequence 5′-AACUCUCCUCACAACUCCCA-3′. In some embodiments, the first guide oligonucleotide comprises a spacer having a sequence that is at least about 75%, at least about 80%, at least about 85%, at least about 90%, at least about 95% identical to, or is identical to 5′-CCAUCACUGGACAUUGCGUC-3′, and the second guide oligonucleotide comprises a spacer having a sequence that is at least about 75%, at least about 80%, at least about 85%, at least about 90%, at least about 95% identical to, or is identical to 5′-AACUCUCCUCACAACUCCCA-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CUGCAUCUGAGCAUCGUGUC-3′; and the second guide oligonucleotide comprises a spacer sequence comprising nucleotides 6 to 20, 5 to 20, 4 to 20, 3 to 20, 2 to 20, or 1 to 20 of the following sequence 5′-CGUCCCUCCGAAUGUUAUUC-3′. In some embodiments, the first guide oligonucleotide comprises a spacer having a sequence that is at least about 75%, at least about 80%, at least about 85%, at least about 90%, at least about 95% identical to, or is identical to 5′-CUGCAUCUGAGCAUCGUGUC-3′, and the second guide oligonucleotide comprises a spacer having a sequence that is at least about 75%, at least about 80%, at least about 85%, at least about 90%, at least about 95% identical to, or is identical to 5′-CGUCCCUCCGAAUGUUAUUC-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-AAACAGCCGUGGACGUCGCA-3′; and the second guide oligonucleotide comprises a spacer sequence comprising nucleotides 6 to 20, 5 to 20, 4 to 20, 3 to 20, 2 to 20, or 1 to 20 of the following sequence 5′-UGAACAAGGUAAGAAGUCUC-3′. In some embodiments, the first guide oligonucleotide comprises a spacer having a sequence that is at least about 75%, at least about 80%, at least about 85%, at least about 90%, at least about 95% identical to, or is identical to 5′-AAACAGCCGUGGACGUCGCA-3′, and the second guide oligonucleotide comprises a spacer having a sequence that is at least about 75%, at least about 80%, at least about 85%, at least about 90%, at least about 95% identical to, or is identical to 5′-UGAACAAGGUAAGAAGUCUC-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-ACAGAGGCUCCUUCUGAACA-3′; and the second guide oligonucleotide comprises a spacer sequence comprising nucleotides 6 to 20, 5 to 20, 4 to 20, 3 to 20, 2 to 20, or 1 to 20 of the following sequence 5′-GCUUGGAACCGGGGCCACUG-3′. In some embodiments, the first guide oligonucleotide comprises a spacer having a sequence that is at least about 75%, at least about 80%, at least about 85%, at least about 90%, at least about 95% identical to, or is identical to 5′-ACAGAGGCUCCUUCUGAACA-3′, and the second guide oligonucleotide comprises a spacer having a sequence that is at least about 75%, at least about 80%, at least about 85%, at least about 90%, at least about 95% identical to, or is identical to 5′-GCUUGGAACCGGGGCCACUG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-AUGCCAGUGUGGUGUCAUAG-3′; and the second guide oligonucleotide comprises a spacer sequence comprising nucleotides 6 to 20, 5 to 20, 4 to 20, 3 to 20, 2 to 20, or 1 to 20 of the following sequence 5′-ACAACAGAAUAUUAUCCAAA-3′. In some embodiments, the first guide oligonucleotide comprises a spacer having a sequence that is at least about 75%, at least about 80%, at least about 85%, at least about 90%, at least about 95% identical to, or is identical to 5′-AUGCCAGUGUGGUGUCAUAG-3′, and the second guide oligonucleotide comprises a spacer having a sequence that is at least about 75%, at least about 80%, at least about 85%, at least about 90%, at least about 95% identical to, or is identical to 5′-ACAACAGAAUAUUAUCCAAA-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-CUAUGACACCACAUUGGCAU-3′; and the second guide oligonucleotide comprises a spacer sequence comprising nucleotides 6 to 20, 5 to 20, 4 to 20, 3 to 20, 2 to 20, or 1 to 20 of the following sequence 5′-ACAUGUUCUUCCUGUGAUAG-3′. In some embodiments, the first guide oligonucleotide comprises a spacer having a sequence that is at least about 75%, at least about 80%, at least about 85%, at least about 90%, at least about 95% identical to, or is identical to 5′-CUAUGACACCACAUUGGCAU-3′, and the second guide oligonucleotide comprises a spacer having a sequence that is at least about 75%, at least about 80%, at least about 85%, at least about 90%, at least about 95% identical to, or is identical to 5′-ACAUGUUCUUCCUGUGAUAG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-AAUAACAUUCGGAGGGACGA-3′; and the second guide oligonucleotide comprises a spacer sequence comprising nucleotides 6 to 20, 5 to 20, 4 to 20, 3 to 20, 2 to 20, or 1 to 20 of the following sequence 5′-UAUUCUGGCUCCAAGCCUAG-3′. In some embodiments, the first guide oligonucleotide comprises a spacer having a sequence that is at least about 75%, at least about 80%, at least about 85%, at least about 90%, at least about 95% identical to, or is identical to 5′-AAUAACAUUCGGAGGGACGA-3′, and the second guide oligonucleotide comprises a spacer having a sequence that is at least about 75%, at least about 80%, at least about 85%, at least about 90%, at least about 95% identical to, or is identical to 5′-UAUUCUGGCUCCAAGCCUAG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-GUAGCAGUCCUGUACCCCGG-3′; and the second guide oligonucleotide comprises a spacer sequence comprising nucleotides 6 to 20, 5 to 20, 4 to 20, 3 to 20, 2 to 20, or 1 to 20 of the following sequence 5′-CAUUAUGGACAGAGUUACCG-3′. In some embodiments, the first guide oligonucleotide comprises a spacer having a sequence that is at least about 75%, at least about 80%, at least about 85%, at least about 90%, at least about 95% identical to, or is identical to 5′-GUAGCAGUCCUGUACCCCGG-3′, and the second guide oligonucleotide comprises a spacer having a sequence that is at least about 75%, at least about 80%, at least about 85%, at least about 90%, at least about 95% identical to, or is identical to 5′-CAUUAUGGACAGAGUUACCG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-AGUAGCAGUCCUGUACCCCG-3′; and the second guide oligonucleotide comprises a spacer sequence comprising nucleotides 6 to 20, 5 to 20, 4 to 20, 3 to 20, 2 to 20, or 1 to 20 of the following sequence 5′-CAUUAUGGACAGAGUUACCG-3′. In some embodiments, the first guide oligonucleotide comprises a spacer having a sequence that is at least about 75%, at least about 80%, at least about 85%, at least about 90%, at least about 95% identical to, or is identical to 5′-AGUAGCAGUCCUGUACCCCG-3′, and the second guide oligonucleotide comprises a spacer having a sequence that is at least about 75%, at least about 80%, at least about 85%, at least about 90%, at least about 95% identical to, or is identical to 5′-CAUUAUGGACAGAGUUACCG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-UAGUAGCAGUCCUGUACCCC-3′; and the second guide oligonucleotide comprises a spacer sequence comprising nucleotides 6 to 20, 5 to 20, 4 to 20, 3 to 20, 2 to 20, or 1 to 20 of the following sequence 5′-CAUUAUGGACAGAGUUACCG-3′. In some embodiments, the first guide oligonucleotide comprises a spacer having a sequence that is at least about 75%, at least about 80%, at least about 85%, at least about 90%, at least about 95% identical to, or is identical to 5′-UAGUAGCAGUCCUGUACCCC-3′, and the second guide oligonucleotide comprises a spacer having a sequence that is at least about 75%, at least about 80%, at least about 85%, at least about 90%, at least about 95% identical to, or is identical to 5′-CAUUAUGGACAGAGUUACCG-3′.

In some embodiments, the first guide oligonucleotide comprises a spacer sequence comprising nucleotides 6 to 20, 5 to 20, 4 to 20, 3 to 20, 2 to 20, or 1 to 20 of the following sequence 5′-UGGACCACAUGGCUUUGCUC-3′; and the second guide oligonucleotide comprises a spacer sequence comprising nucleotides 6 to 20, 5 to 20, 4 to 20, 3 to 20, 2 to 20, or 1 to 20 of the following sequence 5′-ACGUACUCCACCACUGUCAC-3′. In some embodiments, the first guide oligonucleotide comprises a spacer having a sequence that is at least about 75%, at least about 80%, at least about 85%, at least about 90%, at least about 95% identical to, or is identical to 5′-UGGACCACAUGGCUUUGCUC-3′, and the second guide oligonucleotide comprises a spacer having a sequence that is at least about 75%, at least about 80%, at least about 85%, at least about 90%, at least about 95% identical to, or is identical to 5′-ACGUACUCCACCACUGUCAC-3′.

Table 7A summarizes dose response curves in human hepatocellular carcinoma immortalized cells (HuH-7) for five (5) pairs of first and second guide RNAs using a gene editing system that comprises a dual nickase Cas9 system. The Cas9 nickase is encoded within an mRNA (MS029) that was transfected into the HuH-7 cells in a 1:1 total mRNA: total gRNA weight ratio. Table 7B summarizes dose response curves in HuH-7 cells tested in a separate experiment under similar conditions to those used to generate the results presented in Table 7A.

TABLE 7A
Average editing % of selected pairs of guide RNA pairs at different doses

| | Average Editing % | | | | | | | |
|---|---|---|---|---|---|---|---|---|
| | 10000 | 5000 | 2500 | 1250 | 625 | 312.5 | 156.25 | 78.125 |
| Guide pair ID | ng/ml | ng/ml | ng/ml | ng/ml | ng/ml | ng/ml | ng/ml | ng/ml |
| GA1183/GA1184 | 62.4 | 55.2 | 48.7 | 35.5 | 30.8 | 22.5 | 15.4 | 13 |
| GA1227/GA1245 | 62 | 45.7 | 40.6 | 34.3 | 26.3 | 21 | 13 | 10 |
| GA1189/GA1190 | 58.6 | 48.1 | 29.2 | 22.9 | 19.4 | 12 | 7.6 | 6.2 |
| GA1231/GA1250 | 41.3 | 34 | 16.9 | 13.5 | 7.6 | 3.9 | 2.4 | 2.2 |
| GA1234/GA1253 | 36.1 | 22.4 | 15.5 | 13.3 | 10.2 | 6.9 | 4 | 2.5 |

TABLE 7B
Average editing % of selected pairs
of guide RNA pairs at different doses

Average Editing %

| Guide Pair ID | 5000 ng/ml | 2500 ng/ml | 312.5 ng/ml |
|---|---|---|---|
| GA1183/GA1184 | 24.6 | 37.2 | 14.6 |
| GA1264/GA1184 | 33.9 | 38.6 | 16.0 |
| GA1265/GA1184 | 37.1 | 35.1 | 14.1 |
| GA1266/GA1184 | 29.7 | 32.3 | 16.8 |
| GA1076/GA1270 | 25.7 | 27.9 | 10.8 |

In one or more embodiments, a guide oligonucleotide, or a portion(s) thereof, is chemically modified. Chemical modification of the guide oligonucleotide may provide improved stability when transfected into mammalian cells. For example, gRNAs can be chemically modified to comprise a combination of 2′-O-methylribosugar and phosphorothioate backbone modifications on at least one 5′ nucleotide and at least one 3′ nucleotide of each gRNA. In one or more cases, the three terminal 5′ nucleotides and three terminal 3′ nucleotides are chemically modified to comprise combinations of 2′-O-methylribosugar and phosphorothioate modifications.

The present disclosure also contemplates guide nucleic acids that comprise component portions (e.g., crRNA, tracrRNA or scaffold region that may comprise tracrRNA, and connector regions there between) of the guide nucleic acids specified above.

The guide oligonucleotides described herein can be synthesized chemically, enzymatically, or via a combination thereof. For example, the guide oligonucleotide can be synthesized using conventional phosphoramidite-based solid-phase synthesis methods. Alternatively, a gRNA can be synthesized in vitro by operably linking DNA encoding the gRNA to a promoter control sequence that is recognized by, for example, a phage RNA polymerase. Examples of suitable phage promoter sequences include, but are not limited to, T7, T3, SP6 promoter sequences, or variations thereof. In one or more embodiments, a guide oligonucleotide comprises two separate molecules (e.g., crRNA (which comprises the spacer) and tracrRNA or scaffold region, which may comprise tracrRNA). One molecule guide oligonucleotide portion (e.g., tracrRNA or scaffold region, which may comprise tracrRNA) can be chemically synthesized and the other molecule (e.g., crRNA) can be enzymatically synthesized. Portions of the guide oligonucleotide can be ligated to one another via splint ligation or other suitable methods, thereby forming a larger unified guide oligonucleotide. Purification processes, including ion pairing anion exchange and/or reverse phase chromatography processes run alone or in sequence with or without filtration followed by lyophilization and/or aliquoting, may be employed to increase the full-length purity of the guide oligonucleotide post synthesis and/or ligation for use as a suitable drug substance in a pharmaceutical compound.

In some embodiments, the guide oligonucleotide may include, in addition to RNA nucleotides, DNA nucleotides and/or nucleotide analogs. A gRNA may comprise molecules other than RNA.

In embodiments, more than two guide nucleic acids may be used simultaneously to install edits at one or more target genomic locations. Using more than two guide nucleic acids with different sequences may improve editing efficacy.

A gene editing and delivery system as described herein may comprise a guide oligonucleotide having a spacer region engineered to bind to a target sequence on a strand of the human LPA gene opposing a protospacer within the LPA gene. In embodiments, the gene editing system is configured such that the nickase in cooperation with a guide oligonucleotide nicks one strand of the LPA gene within, or in proximity to, the target sequence or the protospacer. In some embodiments, the gene editing system is engineered such that the nickase nicks within 5 nucleotides, within 4 nucleotides, within 3 nucleotides, within 2 nucleotides, within 1 nucleotide, or at the location between the 3′ end of the protospacer and the PAM or at a corresponding location in the target sequence. In embodiments, the gene editing system is engineered such that the gene editing system nicks within 2 or 3 nucleotides of the 3′ end of the protospacer or the corresponding location in the target sequence. In embodiments, the gene editing system is engineered such that the gene editing system nicks (i) within the protospacer and within 2 or 3 nucleotides of the 3′ end of the protospacer or (ii) at the corresponding location in the target sequence.

In some embodiments, the target sequence to which the spacer region of the guide oligonucleotide binds corresponds to (i.e., is complementary to) a protospacer sequence listed in Table 2 or Table 5. In some embodiments, the spacer sequence is identical to, or has at least about 75%, at least about 80%, at least about 85%, at least about 90%, or at least about 95% identity to, a protospacer listed in Table 2 or Table 5. In some embodiments, the guide oligonucleotide includes a spacer sequence that is identical or substantially identical to a protospacer listed in Table 2 or Table 5, or a 3′ portion thereof, with 0, 1, 2, 3, 4, or 5 mismatches. In some embodiments, the spacer region of the guide oligonucleotide comprises a sequence identical to the 15, 16, 17, 18, 19, or twenty 3′-most nucleotides of a protospacer listed in Table 2 or Table 5. For example, a spacer may comprise a sequence identical to the 15, 16, 17, 18, 19, or twenty 3′-most nucleotides of the protospacer in the first row and first column Table 2 (5′-GGAGCCAGAATAACATTCGG-3′), meaning that the spacer may comprise a sequence of 5′-GGAGCCAGAAUAACAUUCGG-3′, 5′-GAGCCAGAAUAACAUUCGG-3′, 5′-AGCCAGAAUAACAUUCGG-3′, 5′-GCCAGAAUAACAUUCGG-3, 5′-CCAGAAUAACAUUCGG-3′, or 5′-CAGAAUAACAUUCGG-3′. In some embodiments, the guide oligonucleotide comprises a spacer having a sequence identical to a protospacer listed in Table 2 or Table 5.

A gene editing and delivery system as described herein may comprise a first guide oligonucleotide and a second guide oligonucleotide. In embodiments, the first guide oligonucleotide binds a first target sequence on a first strand of the human LPA gene, and the second guide oligonucleotide binds a second target sequence on a second strand of the LPA gene. The first target sequence opposes and is complementary to a first protospacer within the LPA gene. The second target sequence opposes and is complementary to a second protospacer within the LPA gene. In embodiments, the gene editing system is engineered such that one or more nickases, in cooperation with the first guide oligonucleotide, nicks one strand of the LPA gene within, or in proximity to, the first target sequence or the first protospacer and, in cooperation with the second guide oligonucleotide, nicks the other strand of the LPA gene within, or in proximity to, the second target sequence or the second protospacer. In embodiments, the gene editing system is engineered such that one or more nickases, in cooperation with the first guide oligonucleotide, nicks a strand of the LPA gene within 5 nucleotides, within 4 nucleotides, within 3 nucleotides, within 2 nucleotides, within 1 nucleotide, or at the location between the 3′ end of the first protospacer and the PAM or at a corresponding location in the first target sequence and, in cooperation with the second guide oligonucleotide, nicks the other strand of the LPA gene within 5 nucleotides, within 4 nucleotides, within 3 nucleotides, within 2 nucleotides, within 1 nucleotide, or at the location between the 3′ end of the second protospacer and the PAM or at a corresponding location in the second target sequence. In embodiments, the gene editing system is engineered such that the gene editing system nicks a strand of the LPA gene within 2 or 3 nucleotides of the 3′ end of the first protospacer or the corresponding location in the first target sequence and nicks the other strand of the LPA gene within 2 or 3 nucleotides of the 3′ end of the second protospacer or the corresponding location in the second target sequence. In embodiments, the gene editing system is engineered such that the gene editing system nicks a strand of the LPA gene (i) within the protospacer and within 2 or 3 nucleotides of the 3′ end of the first protospacer or (ii) at the corresponding location in the first target sequence and nicks the other strand of the LPA gene (i) within the second protospacer and within 2 or 3 nucleotides of the 3′ end of the second protospacer or (ii) at the corresponding location in the second target sequence.

In some embodiments, the target sequence to which the spacer region of the first guide oligonucleotide binds corresponds to a guide 1 protospacer sequence listed in Table 2 or Table 5, and the target sequence to which the spacer region of the second guide oligonucleotide binds corresponds to a guide 2 protospacer sequence listed in Table 2 or Table 5, where the guide 1 protospacer and the guide 2 protospacer are in the same row of Table 2 or Table 5. In some embodiments, the spacer sequence of the first guide oligonucleotide is identical to, or has at least about 75%, at least about 80%, at least about 85%, at least about 90%, or at least about 95% identity to, a guide 1 protospacer listed in Table 2 or Table 5, and the spacer sequence of the second guide oligonucleotide is identical to, or has at least about 75%, at least about 80%, at least about 85%, at least about 90%, or at least about 95% identity to, a guide 2 protospacer listed in Table 2 or Table 5, where the guide 1 protospacer and the guide 2 protospacer are in the same row of Table 2 or Table 5. In some embodiments, the first guide oligonucleotide includes a spacer sequence that is identical or substantially identical to a guide 1 protospacer sequence listed in Table 2 or Table 5, or a 3′ portion thereof, with 0, 1, 2, 3, 4, or 5 mismatches, and the second guide oligonucleotide includes a spacer sequence that is identical or substantially identical to a guide 2 protospacer sequence listed in Table 2 or Table 5, or a 3′ portion thereof, with 0, 1, 2, 3, 4, or 5 mismatches, where the guide 1 protospacer and the guide 2 protospacer are in the same row of Table 2 or Table 5. In some embodiments, the spacer region of the first guide oligonucleotide comprises a sequence identical to the 15, 16, 17, 18, 19, or twenty 3′-most nucleotides of a guide 1 protospacer listed in Table 2 or Table 5, and the spacer region of the second guide oligonucleotide comprises a sequence identical to the 15, 16, 17, 18, 19, or twenty 3′-most nucleotides of a guide 2 protospacer listed in Table 2 or Table 5, where the guide 1 protospacer and the guide 2 protospacer are in the same row of Table 2 or Table 5. In some embodiments, the first guide oligonucleotide comprises a spacer having a sequence identical to a guide 1 protospacer listed in Table 2 or Table 5, and the second guide oligonucleotide comprises a spacer having a sequence identical to a guide 2 protospacer listed in Table 2 or Table 5, where the guide 1 protospacer and the guide 2 protospacer are in the same row of Table 2 or Table 5.

### V. GENE EDITOR SYSTEMS

The term “gene editor” is used throughout this disclosure to refer to a protein or protein complex that is capable of inserting, replacing, deleting, or nicking a DNA sequence in a genome in the presence of, or in operation with, a guide nucleic acid(s). The guide nucleic acid(s) and the gene editor are collectively referred to herein as a “gene editor system” or “gene editing system.” With some gene editing systems, intracellular enzymes, such as DNA repair enzymes, may facilitate or otherwise be required to finalize incorporation of the edit into the genome. In some embodiments, a gene editor that nicks a DNA strand may in operation interact with two different guide oligonucleotides to nick opposing strands of genomic DNA. The nicks on opposing strands may facilitate one or more DNA repair mechanisms to cause the edit (e.g., cause an indel variant or non-synonymous variant).

Gene editor systems are nucleotide-directed. These systems generally include at least an editing protein and a guide nucleic acid. The proteins and systems described above as Cas9, nucleotide-directed editing proteins, and nickases are encompassed by the term “gene editing system”. The guide nucleic acids described above may also be a component of a “gene editing system”.

The editing proteins described herein are nickases, which in operation with a guide oligonucleotide nick a single strand of genomic DNA. For a given nickase to nick opposing strands of the genomic DNA, the nickase operates with a first guide oligonucleotide to nick one strand of the genomic DNA and operates with a second guide oligonucleotide to nick the other strand of the genomic DNA. The first and second guide oligonucleotides comprise spacer sequences complementary to target sequences on opposing strands of the genomic DNA. Such nickases are contrasted with nucleases (e.g., a Cas9 nuclease) that interact with a single guide RNA to catalyze double-strand cleavage of DNA.

The editing proteins described herein may include, but are not limited to, a Cas nickase such as a Streptococcus pyogenes Cas9 variant, a Staphylococcus aureus Cas9 variant, or a Cas12a/Cpf1 variant. The editing protein may be provided as a recombinant protein. The editing protein may alternately be transcribed and/or translated from a provided nucleotide such as mRNA or plasmid DNA.

The guide nucleic acid may include, but is not limited to, a spacer sequence and a scaffold region. The guide nucleic acid components may be covalently attached to one another, may be assembled into a complex, or may be provided as individual strands. The nucleotide components may alternately be transcribed from a provided nucleic acid, such as plasmid DNA.

In one or more embodiments, the nucleotide components and the gene editor may be transcribed from a single nucleic acid, such as plasmid DNA or linear DNA. Protein components may then be translated from a transcript. In one or more embodiments, the nucleic acid encoding the gene editor is an mRNA. In one or more embodiments, the mRNA generates the gene editor upon translation in the targeted cell or subject after the administration. In one or more embodiments, the gene editor forms a ribonucleoprotein (RNP) complex in the targeted cell or subject.

It should be appreciated that the editing of the present disclosure may comprise one or more additional features. For example, in one or more embodiments, the gene editor may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In one or more embodiments, the gene editor comprises one or more His tags.

### VI. THERAPEUTIC APPLICATIONS

The guide nucleic acids and compositions described herein may be administered to target cells or a subject in need thereof, in a therapeutically effective amount, to treat or prevent cardiovascular disease. In one or more embodiments, the subject has cardiovascular disease due at least in part to an elevated blood Lp(a) concentration, which may be directly correlated with apo(a) concentration or may be inversely correlated with size of the apo(a) protein. In one or more embodiments, the subject has atherosclerotic cardiovascular disease due at least in part to an elevated blood Lp(a) concentration, which may be directly correlated with apo(a) concentration or may be inversely correlated with the size of the apo(a) protein. In one or more embodiments, the subject has calcific aortic valve disease due at least in part to an elevated blood Lp(a) concentration, which may be directly correlated with apo(a) concentration or may be inversely correlated the size of the apo(a) protein.

With such administration, the guide nucleic acids direct the gene editor to effect a modification in the LPA gene to reduce the blood Lp(a) concentration, which may be directly correlated with apo(a) concentration or may be inversely correlated with the size of the apo(a) protein in the subject. In one or more embodiments, the genetic alteration occurs in the cells of the liver (hepatocytes) in the subject.

For example, the gene editor system includes an editing protein and guide nucleic acids, which may be introduced and/or expressed in a cell wherein target gene editing is desired, such as, for example, a liver cell (or hepatocyte), thereby allowing contact of the target gene with the guide nucleic acids, such as gRNAs, and the gene editor protein. In one or more embodiments, the binding of the editing protein to the target polynucleotide sequences in the target gene is directed by the guide nucleic acids, wherein the spacer sequence of each guide nucleic acid hybridizes with a complementary sequence in the target strand in the target gene. Thus, the guide nucleic acids guide the editing protein to edit a polynucleotide sequence in the target gene. In one or more embodiments, the guide nucleic acids are co-introduced into a cell where editing is desired with the editing protein or with a nucleic acid encoding the editing protein.

In one or more embodiments, the methods and compositions disclosed herein impair the function of the apo(a) protein encoded by the LPA gene to constitute Lp(a) particles, Impairment of function may be measured by the blood Lp(a) concentration in a subject to whom the method or composition disclosed herein has been administered. For example, the methods and compositions disclosed herein may reduce the blood Lp(a) concentration by at least 10-95 percent relative to a control. For example, the methods and compositions disclosed herein may reduce the blood Lp(a) concentration by at least 10 percent, at least 15 percent, at least 20 percent, at least 25 percent, at least 30 percent, at least 35 percent, at least 40 percent, at least 45 percent, at least 50 percent, at least 55 percent, at least 60 percent, at least 65 percent, at least 70 percent, at least 75 percent, at least 80 percent, at least 85 percent, at least 90 percent, or at least 95 percent relative to a control.

In one or more embodiments, the method for treating or preventing cardiovascular disease in a subject in need thereof as described herein includes administering (i) guide nucleic acids and (ii) a nucleic acid encoding an editing protein to the subject.

In one or more embodiments, the method for treating or preventing cardiovascular disease in a subject in need thereof as described herein includes administering a lipid nanoparticle (LNP) encapsulating or otherwise delivering a (i) guide nucleic acids or nucleic acids encoding the guide nucleic acids and/or (ii) an editing protein comprising a programmable DNA binding domain or a nucleic acid encoding the same, as such guide nucleic acids and editing proteins or nucleic acids encoding the same are described herein. In one or more aspects, the (i) guide nucleic acids or nucleic acids encoding the same and (ii) the editing protein comprising a programmable DNA binding domain or a nucleic acid encoding the same are enclosed in the same LNPs. In one or more aspects, they are enclosed in separate LNPs.

### VII. PHARMACEUTICAL COMPOSITIONS

In one or more aspects, provided herein is a pharmaceutical composition comprising the guide oligonucleotides or the gene editor system as provided herein and a pharmaceutically acceptable carrier or excipient. In one or more aspects, provided herein is a pharmaceutical composition for gene modification comprising guide nucleic acids, such as one or more gRNA, as described herein and an editing protein or a nucleic acid sequence encoding the editing protein and a pharmaceutically acceptable carrier. Pharmaceutical compositions are formulated in a conventional manner using one or more pharmaceutically acceptable inactive ingredients that facilitate processing of the active compounds into preparations that can be used pharmaceutically. Suitable pharmaceutically acceptable additives are generally well known in the art. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition can comprise any suitable molar ratio of guide nucleic acids, such as gRNAs, as described herein or nucleic acid sequences encoding the guide RNAs to each other. In embodiments, the weight ratio of one guide nucleic acid (or a nucleic acid sequence encoding the one guide RNA) to the other guide nucleic acid (or a nucleic acid sequence encoding the other guide RNA) is from 10:1 to 1:10, such as from 5:1 to 1:5, 3:1 to 1:3, or 2:1 to 1:2. In some embodiments, the weight ratio is 1:1 to 1:3. In some embodiments, the weight ratio is about 1:2. In some embodiments, the weight ratio is 1:1.5 to 1:2.5. Thus, for example, the weight ratio of one guide RNA to the other guide RNA may be 1:1 to 1:3 or 3:1.

A pharmaceutical composition can comprise any suitable molar ratio of guide nucleic acids, such as gRNAs, as described herein or nucleic acid sequences encoding the guide RNAs to the editing protein or a nucleic acid sequence encoding the editing protein. In embodiments, the weight ratio of the guide nucleic acids (or nucleic acid sequences encoding the guide nucleic acids) to the editing protein (or nucleic acid encoding the editing protein) is from 10:1 to 1:10, such as from 5:1 to 1:5, 3:1 to 1:3, or 2:1 to 1:2. In some embodiments, the weight ratio is 1:1 to 1:3. In some embodiments, the weight ratio is about 1:2. In some embodiments, the ratio is 1:1.5 to 1:2.5. Thus, for example, the weight ratio of guide RNAs to mRNA encoding the editing protein may be 1:1 to 1:3 or 3:1.

A pharmaceutical composition can be a mixture of guide nucleic acids, such as gRNAs, as described herein or nucleic acid sequences encoding the guide RNAs and an editing protein or a nucleic acid sequence encoding the editing protein with one or more of other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration to an organism or a subject in need thereof.

The pharmaceutical compositions of the present disclosure can be administered to a subject using any suitable methods known in the art. The pharmaceutical compositions described herein can be administered to the subject in a variety of ways, including parenterally, intravenously, intradermally, intramuscularly, colonically, rectally, or intraperitoneally. In one or more embodiments, the pharmaceutical compositions can be administered by intraperitoneal injection, intramuscular injection, subcutaneous injection, or intravenous injection of the subject. In one or more embodiments, the pharmaceutical compositions can be administered parenterally, intravenously, intramuscularly, or orally. In one embodiment, the pharmaceutical composition comprises a pharmaceutically acceptable solution comprising an LNP encapsulating one or more gRNAs and mRNAs encoding the editor protein(s) engineered to effect edits in the LPA gene, as described herein, that is administered intravenously to a subject in need thereof; the LNP may or may not include GalNAc (e.g., a GalNAc-lipid) as described herein.

In one or more embodiments, a pharmaceutical composition for gene modification includes a further therapeutic agent. The additional therapeutic agent may modulate different aspects of the disease, disorder, or condition being treated and provide a greater overall benefit than administration of the therapeutic agent alone. Therapeutic agents include, but are not limited to, a chemotherapeutic agent, a radiotherapeutic agent, a hormonal therapeutic agent, and/or an immunotherapeutic agent. In one or more embodiments, the therapeutic agent may be a radiotherapeutic agent. In one or more embodiments, the therapeutic agent may be a hormonal therapeutic agent. In one or more embodiments, the therapeutic agent may be an immunotherapeutic agent. In one or more embodiments, the therapeutic agent is a chemotherapeutic agent. Preparation and dosing schedules for additional therapeutic agents can be used according to manufacturers' instructions or as determined empirically by a skilled practitioner.

### A. Lipid Nanoparticle (LNP) Compositions

The pharmaceutical compositions for gene modification described herein may be encapsulated in or comprise lipid nanoparticles (LNPs). As used herein, a “lipid nanoparticle (LNP) composition” or a “nanoparticle composition” is a composition comprising one or more described lipids. LNP compositions or formulations, as contemplated herein, are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition or formulation as contemplated herein may be a liposome having a lipid bilayer with a diameter of 500 nm or less. A LNP as described herein may have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 50 nm to 90 nm, from about 55 nm to 85 nm, from about 55 nm to 75 nm, from about 50 nm to about 80 nm, from about 60 nm to about 80 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater.

In one embodiment the mean diameter of the LNP is about 70 nm+/−20 nm, 70 nm+/−10 nm, 70 nm+/−5 nm. In one embodiment the mean diameter of the LNP is about 60 nm+/−20 nm, 60 nm+/−10 nm, 60 nm+/−5 nm. In one embodiment the mean diameter of the LNP is about 50 nm+/−20 nm, 50 nm+/−10 nm, 50 nm+/−5 nm. The LNPs described herein can be substantially non-toxic.

Lipid nanoparticles (LNPs) employ a non-viral drug delivery mechanism that is capable of passing through blood vessels and reaching hepatocytes (Am. J. Pathol. 2010, 176, 14-21). Apolipoprotein E (apoE) proteins are capable of binding to the LNPs post PEG-lipid diffusion from the LNP surface with a near neutral charge in the bloodstream, and thereby function as an endogenous ligand against hepatocytes, which express the low-density lipoprotein receptor (LDLR) (Mol. Ther., 2010, 18, 1357-1364). Requirements for the efficient hepatic delivery of LNP include: 1) effective PEG-lipid shedding from the LNP surface in blood serum and 2) apoE binding to the LNP. The endogenous apoE-mediated LDLR-dependent LNP delivery route is unavailable or a less effective path to achieve LNP-based hepatic gene delivery in patient populations that are LDLR deficient.

Efficient delivery to cells requires specific targeting and substantial protection from the extracellular environment, particularly serum proteins. One method of achieving specific targeting is to conjugate a targeting moiety to an active agent or pharmaceutical effector such as a nucleic acid agent, thereby directing the active agent or pharmaceutical effector to particular cells or tissues depending on the specificity of the targeting moiety. One way a targeting moiety can improve delivery is by receptor-mediated endocytotic activity. This mechanism of uptake involves the movement of nucleic acid agent bound to membrane receptors into the interior of an area that is enveloped by the membrane via invagination of the membrane structure or by fusion of the delivery system with the cell membrane. This process is initiated via activation of a cell surface or membrane receptor following binding of a specific moiety, such as a ligand, to the receptor. Receptor-mediated endocytotic systems include those that recognize sugars such as galactose, mannose, mannose-6-phosphate, peptides and proteins such as transferrin, asialoglycoprotein, vitamin B12, insulin, and epidermal growth factor (EGF). Lipophilic moieties, such as cholesterol or fatty acids, when attached to highly hydrophilic molecules such as nucleic acids can substantially enhance plasma protein binding and consequently circulation half-life. Lipophilic conjugates can also be used in combination with the targeting moieties in order to improve the intracellular trafficking of the targeted delivery approach.

The asialoglycoprotein receptor (ASGPR) is a high-capacity receptor, which is abundant on hepatocytes. ASGPR shows a 50-fold higher affinity for N-Acetyl-D-Galactosamine (GalNAc) than D-Galactose. LNPs comprising receptor-targeting conjugates may be used to facilitate targeted delivery of the drug substances described herein. The LNPs may include one or more receptor-targeting moiety on the surface or periphery of the particle at specified or engineered surface density ranging from relatively low to relatively high surface density. The receptor-targeting conjugate may comprise a targeting moiety (such as a ligand), a linker, and a lipophilic moiety that is connected to the targeting moiety. In one or more embodiments, the receptor-targeting moiety (such as a ligand) targets a lectin receptor. In one or more embodiments, the lectin receptor is asialoglycoprotein receptor (ASGPR). In one or more embodiments the receptor-targeting moiety is GalNAc or a derivative GalNAc that targets ASGPR. In one aspect the receptor-targeting conjugate comprises of one GalNAc moiety or derivative thereof. In another aspect, the receptor-targeting conjugate comprises two different GalNAc moieties or derivatives thereof. In another aspect, the receptor-targeting conjugate comprises three different GalNAc moieties or derivatives thereof In another aspect, the receptor-targeting conjugate is lipophilic. In one or more embodiments, the receptor-targeting conjugate comprises one or more GalNAc moieties and one or more lipid moieties, i.e., GalNAc-Lipid. In one or more embodiments, the receptor-targeting conjugate is a GalNAc-Lipid.

Described herein are (i) LNP compositions comprising an amino lipid, a phospholipid, a PEG lipid, a cholesterol, or a derivative of a cholesterol, a payload, or any combination thereof and (ii) LNP compositions comprising an amino lipid, a phospholipid, a PEG-lipid, a cholesterol, a GalNAc-Lipid or a derivative thereof, a payload, or any combination thereof. Each component is described in more detail below.

In the preparation of LNP compositions comprising the excipients amino lipid, phospholipid, PEG-Lipid, and cholesterol, a desired molar ratio of the four excipients is dissolved in a water-miscible organic solvent, e.g., ethanol. The homogenous lipid solution is then rapidly in-line mixed with an aqueous buffer with acidic pH ranging from 4 to 6.5 containing nucleic acid payload to form the lipid nanoparticle (LNP) encapsulating the nucleic acid payload(s). After rapid in-line mixing the LNPs thus formed undergo further downstream processing including concentration and buffer exchange to achieve the final LNP pharmaceutical composition with near neutral pH for administration into a cell line or animal disease model for evaluation, or to administer to human subjects.

For the preparation of GalNAc-LNP pharmaceutical composition, the GalNAc-Lipid is mixed with the four lipid excipients in the water-miscible organic solvent prior to the preparation of the GalNAc-LNP. The preparation of the GalNAc-LNP pharmaceutical composition then follow the same steps as described for the LNP pharmaceutical composition. The mol % of the GalNAc-Lipid in the GalNAc-LNP preparation ranges from 0.001 to 2.0 of the total excipients.

For both LNP and GalNAc-LNP preparation, the payload comprises guide nucleic acids, such as guide RNAs, targeting the LPA gene and an mRNA encoding a gene editor protein. In one or more embodiments, the guide nucleic acid to mRNA weight ratio in the acidic aqueous buffer and in the final formulation is 6:1, 5:1, 4:1, 3:1, 2.5:1, 2:1, 1.5:1, 1:1, 1:1.5, 1:2, 1:2.5, 1:3, 1:5, or 1:6 by weight.

In one or more embodiments, an LNP composition may be prepared as described in U.S. patent application Ser. No. 17/192,709, entitled COMPOSITIONS AND METHODS FOR TARGETED RNA DELIVERY, filed on 4 Mar. 2021, claiming the benefit of U.S. Provisional Patent Application Nos. 62/984,866 (filed on 4 Mar. 2020) and 63/078,982 (filed on 16 Sep. 2020), naming Kallanthottathil G. Rajeev as an inventor and Verve Therapeutics, Inc., as the applicant, which application is hereby incorporated herein by reference in its entirety.

### 1. Amino Lipids

### a) Formula (I)

In one or more embodiments, the LNP composition comprises an amino lipid. In one aspect, disclosed herein is an amino lipid having the structure of Formula (I), or a pharmaceutically acceptable salt or solvate thereof,

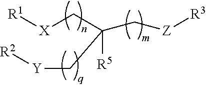

wherein
each of R¹ and R² is independently C₃-C₂₂ alkyl, C₃-C₂₂ alkenyl, C₃-C₈ cycloalkyl, —C₂-C₁₀ allkylene-L-R⁶, or

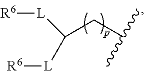

wherein each of the alkyl, alkylene, alkenyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—. —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴— O, S, or a bond;
each of L is independently —C(═O)NR⁴—, —NR⁴C(═O)—, —C(═O)O—. —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaiyl is independently substituted or unsubstituted; each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and Rx is independently hydrogen or substituted or unsubstituted C₁-C₆ alkyl, or R⁷ and R⁸ taken together with the nitrogen to which they are attached form a substituted or unsubstituted C₂-C₆ heterocyclyl;
p is an integer selected from 1 to 10; and
each of n, m, and q is independently 0, 1, 2, 3, 4, or 5.

In one or more embodiments of Formula (I), if the structure carries more than one asymmetric C— atom, each asymmetric C-atom independently represents racemic, chirally pure R and/or chirally pure S isomer, or a combination thereof.

In one or more embodiments, each of n, in, and q in Formula (I) is independently 0, 1, 2, or 3. In one or more embodiments, each of n, m, and q in Formula (I) is 1.
b) Formula (Ia)

In one or more embodiments, the compound of Formula (I) has a structure of Formula (Ia), or a pharmaceutically acceptable salt or pharmaceutically acceptable solvate thereof:

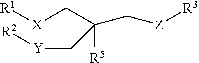

wherein
each of R¹ and R² is independently C₃-C₂₂ alkyl, C₃-C₂₂ alkenyl, C₃-C₈ cycloalkyl, —C₂-C₁₀ alkylene-L-R⁶, or

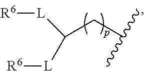

wherein each of the alkyl, alkylene, alkenyl, and cycloalkyl is independently substituted or unsubstituted;
each of X, Y, and Z is independently C(═O)NR⁴—, —NR⁴C(O)—, —C(═O)O—, —OC(═O)—, —OC(═O)O—, —NR⁴C(═O)O—, —OC(═O)NR⁴—, —NR⁴C═O)NR⁴—, —NR⁴C(═NR⁴)NR⁴—. —C(═S)NR⁴—, —NR⁴C(═S)—, —C(E)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—. —SC(═S)—, —SC(═O)S—, —SC(═S)S—. —NR⁴C(═S)S—, —SC(═S)NR⁴—, O, S, —C₁-C₁₀ alkylene-O—, or a bond, wherein the alkylene is substituted or unsubstituted;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—. —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S. —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted; R³ is —C₀-C₁₀ alkylene-NR⁷R⁸, —C₀-C₁₀ alkylene-heterocycloalkyl, or —C₀-C₁₀ alkylene-heterocyclowyl, wherein the alkylene, heterocycloalkyl and heterocycloaryl is independently substituted or unsubstituted;
each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl; and
p is an integer selected from 1 to 10.

In one or more embodiments of Formula (Ia), if the structure carries more than one asymmetric C-atom, each asymmetric C-atom independently represents racemic, chirally pure R and/or chirally pure S isomer, or a combination thereof.
c) Variations of Formula (I) and (Ia)

In one or more embodiments, R¹ and R² in Formula (I) and Formula (Ia) is independently C₃-C₂₂ alkyl, C₃-C₂₂ alkenyl, —C₂-C₁₀ alkylene-L- R⁶, or

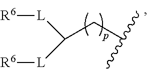

wherein each of the alkyl, alkylene, alkenyl, and cycloalkyl is independently substituted or unsubstituted. In one or more embodiments, R¹ and R² in Formula (I) and Formula (Ia) is independently C₁₀-C₂₀ alkyl, C₁₀-C₂₀ alkenyl, —C₈-C₇ alkylene-L- R⁶, or

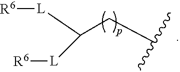

wherein each of the alkyl, alkylene, alkenyl, and cycloalkyl is independently substituted or unsubstituted. In one or more embodiments, R¹ in Formula (I) and Formula (Ia) is

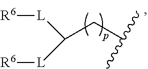

In one or more embodiments, each of L in Formula (I) and Formula (Ia) is independently O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted. In one or more embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is substituted or unsubstituted. In one or more embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is linear or branched unsubstituted alkylene.

In one or more embodiments, each of R⁶ in Formula (I) and Formula (Ia) is independently substituted or unsubstituted linear C₃-C₂₂ alkyl or substituted or unsubstituted linear C₃-C₂₂ alkenyl. In one or more embodiments, each of R⁶ in Formula (I) and Formula (Ia) is independently substituted or unsubstituted C₃-C₂₀ alkyl or substituted or unsubstituted C₃-C₂₀ alkenyl. In one or more embodiments, each of R⁶ in Formula (I) and Formula (Ia) is independently substituted or unsubstituted C₃-C₁₀ alkyl or substituted or unsubstituted C₃-C₁₀ alkenyl. In one or more embodiments, each of R⁶ in Formula (I) and Formula (Ia) is independently substituted or unsubstituted C₃-C₁₀ alkyl. In one or more embodiments, each of R⁶ in Formula (I) and Formula (Ia) is independently substituted or unsubstituted linear C₃-C₁₀ alkyl. In one or more embodiments, each of R⁶ in Formula (I) and Formula (Ia) is independently substituted or unsubstituted n-pentyl, n-hexyl, n-heptyl, n-octyl, n-nonyl, n-decyl, n-undecyl, or n-dodecyl. In one or more embodiments, each of R⁶ in Formula (I) and Formula (Ia) is independently substituted or unsubstituted n-octyl. In one or more embodiments, each of R⁶ in Formula (I) and Formula (Ia) is n-octyl.

In one or more embodiments, each ofL in Formula (I) and Formula (Ia) is independently —C(═O)O—, —OC(═O)—, —C₁-C₁₀ alkylene-O—, or O. In one or more embodiments, each of L in Formula (I) and Formula (Ia) is O. In one or more embodiments, each of L in Formula (I) and Formula (Ia) is —C₁-C₃ alkylene-O—. In one or more embodiments, p in Formula (I) and Formula (Ia) is 1, 2, 3, 4, or 5. In one or more embodiments, p in Formula (I) and Formula (Ia) is 2.

In one or more embodiments, R¹ in Formula (I) and Formula (Ia) is

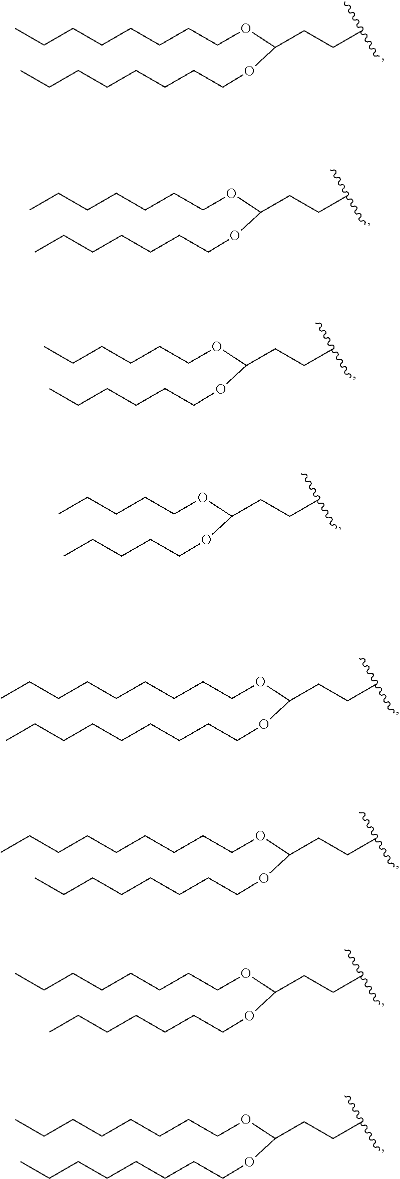

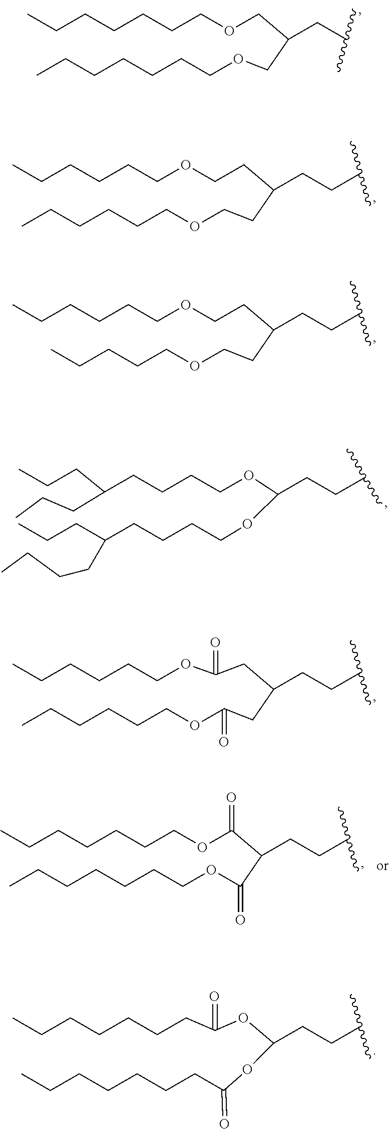

In one or more embodiments R¹ in Formula (I) and Formula (Ia) is R².

In one or more embodiments, each of R⁴ in Formula (I) and Formula (Ia) is independently H or substituted or unsubstituted C₁-C₄ alkyl. In one or more embodiments, each of. R⁴ in Formula (I) and Formula (Ia) is independently substituted or unsubstituted linear C₁-C₄ alkyl. In one or more embodiments, each of R⁴ in Formula (1) and Formula (Ia) is H. In one or more embodiments, each of R⁴ in Formula (I) and Formula (Ia) is independently H, —CH₃, —CH₂CH₃, —CH₂CH₂CH₃, or —CH(CH₃)₂. In one or more embodiments, each of R⁴ in Formula (I) and Formula (Ia) is independently H or —CH₃. In one or more embodiments, each of R⁴ in Formula (I) and Formula (Ia) is —CH₃.

In one or more embodiments, X in Formula (I) and Formula (Ia) is —C(═O)O— or —OC(═O))—. In one or more embodiments, X in Formula (I) and Formula (Ia) is —C(═O)NR⁴— or —NR⁴C(═O)—. In one or more embodiments, X in Formula (I) and Formula (Ia) is —C(═O)N(CH₃)—, —N(CH₃)C(═O)—, —C(═O)NH—, or —NHC(═O)—. In one or more embodiments, X in Formula (I) and Formula (Ia) is —C(═O))NH—, —C(═O)N(CH₃)—. —OC(═O))—, —NHC(═O)—, —N(CH₃)C(═O))—, —C(═O)O—, —OC(═O)O—, —NHC(═O)O—, —N(CH₃)C(═O)O—, —OC(═O))NH—, —OC(═O)N(CH₃)—, —NHC(═O)NH—, —N(CH₃)C(═O))NH—, —NHC(═O)N(CH₃)—, —N(CH₃)C(═O)N(CH₃)—, NHC(═NH)NH—, —N(CH₃)C(═NH)NH—, —NHC(═NH)N(CH₃)—, —N(CH₃)C(═NH)N(CH₃)—, NHC(═NMe)NH—, —N(CH₃)C(═NMe)NH—, —NHC(═NMe)N(CH₃)—, or —N(CH₃)C(═NMe)N(CH₃)—.

In one or more embodiments. R² in Formula (I) and Formula (Ia) is C₃-C₂₂ alkyl, C₃-C₂₂alkenyl, —C₂-C₁₀ alkylene-L-R⁶, or

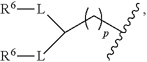

wherein each of the alkyl, alkylene, alkenyl, and cycloalkyl is independently substituted or unsubstituted. In one or more embodiments, R² in Formula (I) and Formula (Ia) is substituted or unsubstituted C₇-C₂₂ alkyl or substituted or unsubstituted C₃-C₂₂ alkenyl. In one or more embodiments, R² in Formula (I) and Formula (Ia) is substituted or unsubstituted linear C₇-C₂₂ alkyl or substituted or unsubstituted linear C₃-C₂₂ alkenyl. In one or more embodiments, R² in Formula (I) and Formula (Ia) is substituted or unsubstituted C₁₀-C₂₀ alkyl or substituted or unsubstituted C₁₀-C₂₀ alkenyl. In one or more embodiments, R² in Formula (I) and Formula (Ia) is unsubstituted C₁₀-C₂₀ alkyl. In one or more embodiments, R² in Formula (I) and Formula (Ia) is unsubstituted C₁₀-C₂₀ alkenyl. In one or more embodiments, R² in Formula (I) and Formula (Ia) is —C₂-C₁₀ alkylene-L- R⁶. In one or more embodiments, R² in Formula (I) and Formula (Ia) is —C₂-C₁₀ alkylene- C(═O)O— R⁶ or —C₂-C₁₀ alkylene-OC(═O)— R⁶.

In one or more embodiments, R² in Formula (I) and Formula (Ia) is

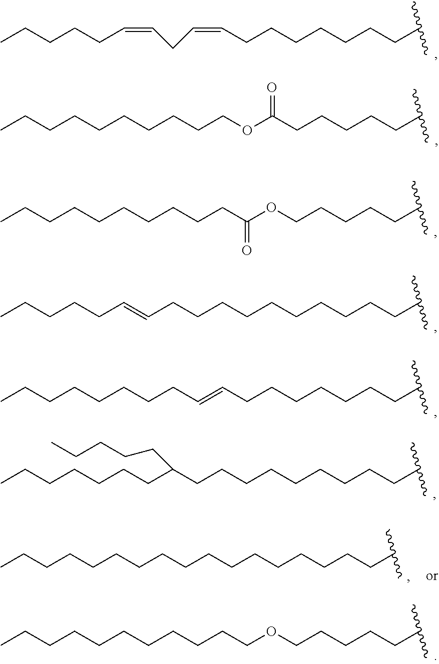

In one or more embodiments, R¹ in Formula (I) and Formula (Ia) is R¹.

In one or more embodiments, Y in Formula (I) and Formula (Ia) is —C(═O)NR⁴— or —OC(═O)—. In one or more embodiments, Y in Formula (I) and Formula (Ia) is —C(═O)N(CH₃)—, —N(CH₃)C(═O)—, —C(═O)NH—, or —NC(═O)—. In one or more embodiments, Y in Formula (I) and Formula (Ia) is —OC(═O)O—, —NR⁴C(═O)O—, —OC(═O)NR⁴—, or —NR⁴C(═O)NR⁴—. In one or more embodiments, Y in Formula (I) and Formula (Ia) is —OC(═O)O—, —NHC(═O)O—, —OC(═O)NH—, —NHC(═O)NH—, —N(CH₃)C(═O)O—. —OC(═O)N(CH₃)—, —N(CH₃)C(═O)N(CH₃)— or —N(CH₃)C(═O)NH—. In one or more embodiments, Y in Formula (I) and Formula (Ia) is —OC(═O)O—, —NHC(═O)O—, —OC(═O)NH—, or —NHC(═O)NH—.

In one or more embodiments, R³ in Formula (I) and Formula (Ia) is —C₀-C₁₀ alkylene-NR⁷ R⁸ or —C₀-C₁₀ alkylene-heterocycloalkyl, wherein the alkylene and heterocycloalkyl is independently substituted or unsubstituted. In one or more embodiments, R³ in Formula (I) and Formula (Ia) is —C₀-C₁₀ alkylene-NR⁷R⁸. In one or more embodiments, R³ in Formula (I) and Formula (Ia) is —C₁-C₆ alkylene-NR⁷R⁸. In one or more embodiments, R³ in Formula (I) and Formula (Ia) is —C₁-C₄ alkylene-NR⁷R⁸. In one or more embodiments, R³ in Formula (I) and Formula (Ia) is —C₁— alkylene-NR⁷R⁸. In one or more embodiments, R³ in Formula (I) and Formula (Ia) is —C₂— alkylene-NR⁷R⁸. In one or more embodiments, R³ in Formula (I) and Formula (Ia) is —C₃— alkylene-NR⁷R⁸. In one or more embodiments, R³ in Formula (I) and Formula (Ia) is —C₄— alkylene- NR⁷R⁸. In one or more embodiments, R³ in Formula (I) and Formula (Ia) is —C₅— alkylene-NR⁷R⁸. In one or more embodiments, R³ in Formula (I) and Formula (Ia) is —C₀-C₁₀ alkylene-heterocycloalkyl. In one or more embodiments, R³ in Formula (I) and Formula (Ia) is —C₁-C₆ alkylene-heterocycloalkyl, wherein the heterocycloalkyl comprises 1 to 3 nitrogen and 0-2 oxygen. In one or more embodiments, R³ in Formula (I) and Formula (Ia) is —C₁-C₆ alkylene-heterocycloaryl.

In one or more embodiments, each of R⁷ and R⁸ in Formula (I) and Formula (Ia) is independently hydrogen or substituted or unsubstituted C₁-C₆ alkyl. In one or more embodiments, each of R⁷ and R⁸ is independently hydrogen or substituted or unsubstituted C₁-C₃ alkyl. In one or more embodiments, each of R⁷ and R⁸ is independently substituted or unsubstituted C₁-C₃ alkyl. In one or more embodiments, each of R⁷ and R⁸ is independently —CH₃, —CH₂CH₃, —CH₂CH₂CH₃, or —CH(CH₃)₂. In one or more embodiments, each of R and R⁸ is CH₃. In one or more embodiments, each of R⁷ and R⁸ is —CH₂CH₃.

In one or more embodiments, R⁷ and R⁸ in Formula (I) and Formula (Ia) taken together with the nitrogen to which they are attached form a substituted or unsubstituted C₂-C₆ heterocyclyl. In one or more embodiments, R⁷ and R⁸ taken together with the nitrogen to which they are attached form a substituted or unsubstituted C₂-C₆ heterocycloalkyl. In one or more embodiments, R⁷ and R⁸ taken together with the nitrogen to which they are attached form a substituted or unsubstituted 3-7 membered heterocycloalkyl.

In one or more embodiments, R³ in Formula (I) and Formula (Ia) is

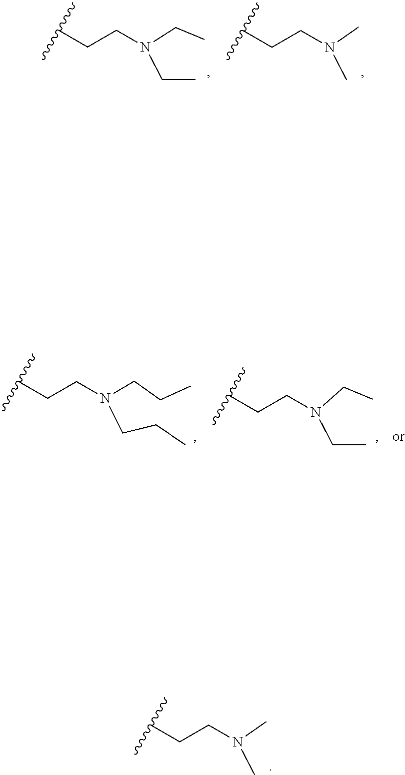

In one or more embodiments. R³ in Formula (I) and Formula (Ia) is

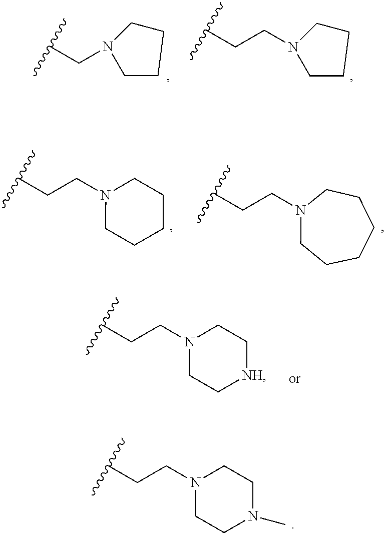

In one or more embodiments. R³ in Formula (1) and Formula (Ia) is

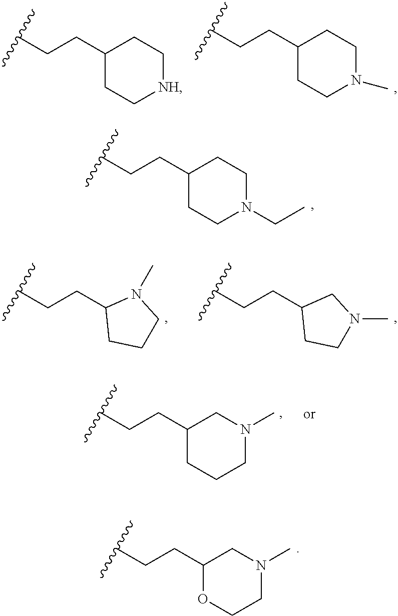

In one or more embodiments, Z in Formula (I) and Formula (Ia) is —C(═O)O— or —OC(═O)—.

In one or more embodiments, Z in Formula (I) and Formula (Ia) is —C(═O)NR⁴— or —NR⁴C(═O)—.

In one or more embodiments, Z in Formula (I) and Formula (Ia) is —C(═O)N(CH₃)—, —N(CH₃)C(═O)—, —C(═O)NH—, or —NHC(═O)—.

In one or more embodiments, Z in Formula (I) and Formula (Ia) is —OC(═O)O—, —NR⁴C(═O)O—, —OC(O)NR⁴—, or —NR⁴C(═O)NR⁴—.

In one or more embodiments, Z in Formula (I) and Formula (Ia) is —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)—, —NHC(═O)N(CH₃)— or —N(CH₃)C(═O)NH—.

In one or more embodiments, Y in Formula (I) and Formula (Ia) is —OC(═O)O—, —NHC(═O)O—, —OC(═O)NH—, or —NHC(═O)NH—.

In one or more embodiments, R⁵ in Formula (I) and Formula (a) is hydrogen or substituted or unsubstituted C₁-C₃ alkyl.

In one or more embodiments, R⁵ in Formula (I) and Formula (Ia) is H, —CH₃, —CH—)CH₃, —CH₂CH₂CH₃, or —CH(CH₃)₂.

In one or more embodiments, R⁵ in Formula (I) and Formula (Ia) is H.

### 2. LNP Compositions Comprising Different Amino Lipids

In one or more embodiments, the LNP comprises a plurality of amino lipids having different formulas. For example, the LNP composition can comprise 2, 3, 4, 5, 6, 7, 8, 9, 10, or more amino lipids. For another example, the LNP composition can comprise at least 2, at least 3, at least 4, at least 5, at least 6, at least 7, at least 8, at least 9, at least 10, or at least 20 amino lipids. For yet another example, the LNP composition can comprise at most 2, at most 3, at most 4, at most 5, at most 6, at most 7, at most 8, at most 9, at most 10, at most 20, or at most 30 amino lipids.

In one or more embodiments, the LNP composition comprises a first amino lipid. In one or more embodiments, the LNP composition comprises a first amino lipid and a second amino lipid. In one or more embodiments, the LNP composition comprises a first amino lipid, a second amino lipid, and a third amino lipid. In one or more embodiments, the LNP composition comprises a first amino lipid, a second amino lipid, a third amino lipid, and a fourth amino lipid. In one or more embodiments, the LNP composition does not comprise a fourth amino lipid. In one or more embodiments, the LNP composition does not comprise a third amino lipid. In one or more embodiments, a molar ratio of the first amino lipid to the second amino lipid is from about 0.1 to about 10. In one or more embodiments, a molar ratio of the first amino lipid to the second amino lipid is from about 0.20 to about 5. In one or more embodiments, a molar ratio of the first amino lipid to the second amino lipid is from about 0.25 to about 4. In one or more embodiments, a molar ratio of the first amino lipid to the second amino lipid is about 0.25, about 0.33, about 0.5, about 1, about 2, about 3, or about 4.

In one or more embodiments, a molar ratio of the first amino lipid: the second amino lipid: the third amino lipid is about 4:1:1. In one or more embodiments, a molar ratio of the first amino lipid: the second amino lipid: the third amino lipid is about 1:1:1. In one or more embodiments, a molar ratio of the first amino lipid: the second amino lipid: the third amino lipid is about 2:1:1. In one or more embodiments, a molar ratio of the first amino lipid: the second amino lipid: the third amino lipid is about 2:2:1. In one or more embodiments, a molar ratio of the first amino lipid: the second amino lipid: the third amino lipid is about 3:2:1. In one or more embodiments, a molar ratio of the first amino lipid: the second amino lipid: the third amino lipid is about 3:1:1. In one or more embodiments, a molar ratio of the first amino lipid: the second amino lipid: the third amino lipid is about 5:1:1. In one or more embodiments, a molar ratio of the first amino lipid: the second amino lipid: the third amino lipid is about 3:3:1. In one or more embodiments, a molar ratio of the first amino lipid: the second amino lipid: the third amino lipid is about 4:4:1.

### 3. Additional Amino Lipid Embodiments

In one or more embodiments, the LNP composition comprises one or more amino lipids. In one or more embodiments, the one or more amino lipids comprise from about 40 mol % to about 65 mol % of the total lipid present in the particle. In one or more embodiments, the one or more amino lipids comprise about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, or about 65 mol % of the total lipid present in the particle. In one or more embodiments, the first amino lipid comprises from about 1 mol % to about 99 mol % of the total amino lipids present in the particle. In one or more embodiments, the first amino lipid comprises from about 16.7 mol % to about 66.7 mol % of the total amino lipids present in the particle. In one or more embodiments, the first amino lipid comprises from about 20 mol % to about 60 mol % of the total amino lipids present in the particle.

In one or more embodiments, the amino lipid is an ionizable lipid. An ionizable lipid can comprise one or more ionizable nitrogen atoms. In one or more embodiments, at least one of the one or more ionizable nitrogen atoms is positively charged. In one or more embodiments, at least 10 mol %, 20 mol %, 30 mol %, 40 mol %, 50 mol %, 60 mol %, 70 mol %, 80 mol %. 90 mol %, 95 mol %, or 99 mol % of the ionizable nitrogen atoms in the LNP composition are positively charged. In one or more embodiments, the amino lipid comprises a primary amine, a secondary amine, a tertiary amine, an imine, an amide, a guanidine moiety, a histidine residue, a lysine residue, an arginine residue, or any combination thereof. In one or more embodiments, the amino lipid comprises a primary amine, a secondary amine, a tertiary amine, a guanidine moiety, or any combination thereof. In one or more embodiments, the amino lipid comprises a tertiary amine.

In one or more embodiments, the amino lipid is a cationic lipid. In one or more embodiments, the amino lipid is an ionizable lipid. In one or more embodiments, the amino lipid comprises one or more nitrogen atoms. In one or more embodiments, the amino lipid comprises one or more ionizable nitrogen atoms. Exemplary cationic and/or ionizable lipids include, but are not limited to, 3-(didodecylamino)-N1,N1,4-tri dodecyl-1-piperazineethan amine (KL10), N142-(didodecylamino)ethy1]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC 3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3p)- cholest-5-en-3-yloxy]octyl}oxy)-N,N-dimethy1-3-[(9Z,12Z)-octadeca-9,12-dien-1-yloxy]propan-1-amine (Octyl-CLinDMA), (2R)-2-({8-[(33)-cholest-5-en-3-yloxy]octyl}oxy)-N,N-dimethy1-3-[(9Z,12Z)-octadeca-9,12-dien-1-yloxy]propan-1-amine (Octyl-CLinDMA (2R)), and (2S)-2-({8-[(3 P)-cholest-5-en-3-yloxy]octyl}oxy)-N,N-dimethy1-3-[(9Z,12Z)-octadeca-9,12-dien-1-yloxy]propan-1-amine (Octyl-CLinDMA (2S)).

In one or more embodiments, an amino lipid described herein can take the form of a salt, such as a pharmaceutically acceptable salt. All pharmaceutically acceptable salts of the amino lipid are encompassed by this disclosure. As used herein, the term “amino lipid” also includes its pharmaceutically acceptable salts, and its diastereomeric, enantiomeric, and epimeric forms.

In one or more embodiments, an amino lipid described herein possesses one or more stereocenters, and each stereocenter exists independently in either the R or S configuration. The lipids presented herein include all diastereomeric, enantiomeric, and epimeric forms as well as the appropriate mixtures thereof. The lipids provided herein include all cis, trans, syn, anti, entgegen (E), and zusammen (Z) isomers as well as the appropriate mixtures thereof. In certain embodiments, lipids described herein are prepared as their individual stereoisomers by reacting a racemic mixture of the compound with an optically active resolving agent to form a pair of diastereoisomeric compounds/salts, separating the diastereomers and recovering the optically pure enantiomers. In one or more embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming of diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In one aspect, stereoisomers are obtained by stereoselective synthesis.

In one or more embodiments, the lipids such as the amino lipids are substituted based on the structures disclosed herein. In one or more embodiments, the lipids such as the amino lipids are unsubstituted. In another embodiment, the lipids described herein are labeled isotopically (e.g., with a radioisotope) or by another other means, including, but not limited to, the use of chromophores or fluorescent moieties, bioluminescent labels, or chemiluminescent labels.

Lipids described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present lipids include isotopes of hydrogen, carbon, nitrogen, oxygen, sulfur, fluorine, and chlorine, such as, for example, ²H, ³H, ¹³C, ¹⁴C, ¹⁵N, ¹⁸O, ¹⁷O, S, ¹⁸F, and ³⁶C₁. In one aspect, isotopically-labeled lipids described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In one or more embodiments, the asymmetric carbon atom of the amino lipid is present in enantiomerically enriched form. In certain embodiments, the asymmetric carbon atom of the amino lipid has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (S)- or (R)-configuration.

In one or more embodiments, the disclosed amino lipids can be converted to N-oxides. In one or more embodiments, N-oxides are formed by a treatment with an oxidizing agent (e.g., 3-chloroperoxybenzoic acid and/or hydrogen peroxides). Accordingly, disclosed herein are N-oxide compounds of the described amino lipids, when allowed by valency and structure, which can be designated as NO or N⁺-O⁻. In one or more embodiments, the nitrogen in the compounds of the disclosure can be converted to N-hydroxy or N-alkoxy. For example, N-hydroxy compounds can be prepared by oxidation of the parent amine by an oxidizing agent such as ra-CPBA. All shown nitrogen-containing compounds are also considered. Accordingly, also disclosed herein are N-hydroxy and N-alkoxy (e.g., N—OR, wherein R is substituted or unsubstituted C₁-C₆ alkyl, C₁-C₆ alkenyl, C₁-C₆ alkynyl, 3-14-membered carbocycle, or 3-14-membered heterocycle) derivatives of the described amino lipids.

In one or more embodirents, the one or more amino lipids comprise from about 40 mol % to about 65 mol % of the total lipid present in the particle.

### 4. PEG-Lipids

In one or more embodiments, the described LNP composition includes one or more PEG-lipids. As used herein, a “PEG lipid” or “PEG-lipid” refers to a lipid comprising a polyethylene glycol component. Examples of suitable PEG-lipids also include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof.

In one or more embodiments, PEG-lipid comprises from about 0.1 mol % to about 10 mol % of the total lipid present in the particle.

### 5. Phospholipid

In one or more embodiments, the described LNP composition includes one or more phospholipids.

In one or more embodiments, the phospholipid comprises from about 5 mol % to about 15 mol % of the total lipid present in the particle.

### 6. Cholesterol

In one or more embodiments, the LNP composition includes a cholesterol or a derivative thereof.

### 7. GalNAc-Lipid

In one or more embodiments, the LNP composition includes a receptor targeting conjugate comprising a compound formula (V),

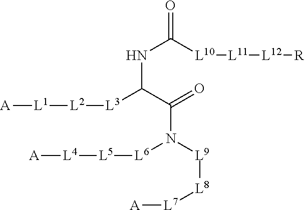

wherein,
a plurality of the A groups collectively comprise a receptor targeting moiety;
each L¹, L², L³, L⁴, L⁵, L⁶, L⁷, L⁸, L⁹, L¹⁰ and L¹² is independently substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, —(CH₂CH₂O)ₘ—, —(OCH₂CH₂)ₘ—, —O—, —S—, —S(═O)—, —S(═O)₂—, —S(═O)(═NR¹)—, —C(═O)—, —C(═N—OR¹)—, —C(═O)O—, —OC(═O)—, —C(═O)C(═O)—, —C(═O)NR¹—, —NR¹C(═O)—, —OC(═O)NR¹—, —NR¹C(═O)O—, —NR¹C(═O)NR¹—, —C(═O)NR¹C(═O)—, —S(═O)₂NR¹—, —NR¹S(═O)₂—, —NR¹—, or —N(OR¹)—;
L¹¹ is substituted or unsubstituted —(CH₂CH₂O)ₙ—, substituted or unsubstituted —(OCH₂CH₂)ₙ— or substituted or unsubstituted —(CH₂)ₙ—;
each R¹ is independently H or substituted or unsubstituted C₁-C₆alkyl;
R is a lipid, nucleic acid, amino acid, protein, or lipid nanoparticle;
m is an integer selected from 1 to 10; and
n is an integer selected from 1 to 200.

In one or more embodiments, each L¹, L⁴, and L⁷ is independently substituted or unsubstituted C₁-C₁₂ alkylene. In one or more embodiments, each L¹, L⁴, and L⁷ is independently substituted or unsubstituted C₂-C₆ alkylene. In one or more embodiments, each L¹, L⁴, and L⁷ is C₄ alkylene. In one or more embodiments, each L², L⁵, and L⁸ is independently —C(═O)NR¹—, —NR¹C(═O)—, —OC(═O)NR¹—, —NR¹C(═O)O—, —NR¹C(═O)NR¹—, or —C(═O)NR¹C(═O)—. In one or more embodiments, each L², L⁵, and L⁸ is independently —C(═O)NR‘- or —NR’C(═O)—. In one or more embodiments, each L², L¹, and L⁸ is —C(═O)NH—. In one or more embodiments, each L³, L⁶, and L⁹ is independently substituted or unsubstituted C₁-C₁₂ alkylene. In one or more embodiments, each L³ is substituted or unsubstituted C₂-C₆ alkylene. In one or more embodiments, L³ is C₄ alkylene. In one or more embodiments, each L⁶ and L⁹ is independently substituted or unsubstituted C₂-C₁₀ alkylene. In one or more embodiments, each L⁶ and L⁹ is independently substituted or unsubstituted C₂-C₆ alkylene. In one or more embodiments, each L⁶ and L⁹ is C₃ alkylene. In one or more embodiments, A binds to a lectin. In one or more embodiments, the lectin is an asialoglycoprotein receptor (ASGPR). In one or more embodiments, A is N-acetylgalactosamine (GalNAc) or

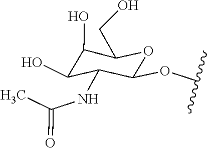 [SMILES: *OC1OC(CO)C(O)C(O)C1NC(C)=O]

or a derivative thereof. A is N-acetylgalactosamine (GalNAc)

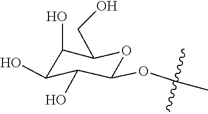 [SMILES: *OC1OC(CO)C(O)C(O)C1O]

or a derivative thereof.

Examples of such GalNAc lipids include the following:

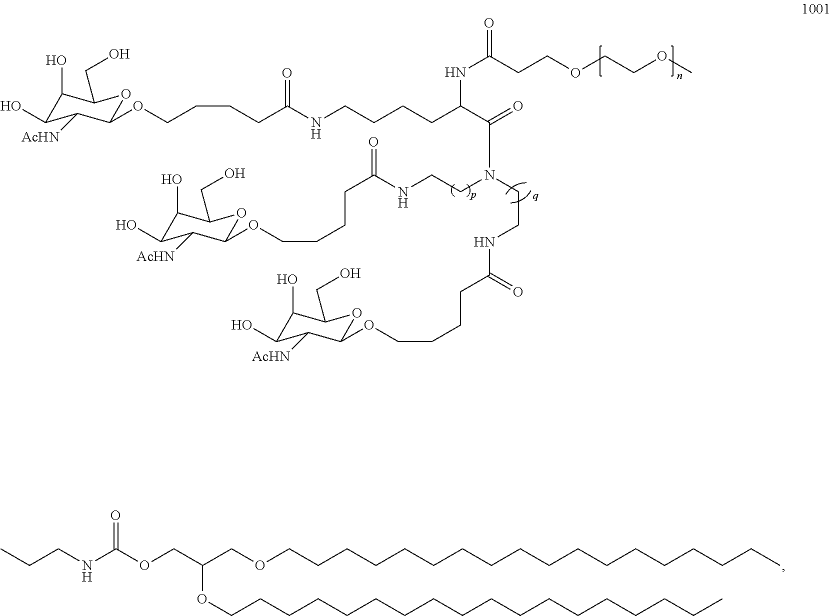

wherein each of the p and q is independently an integer from 1 to 5, and n is an integer from 1 to 50;

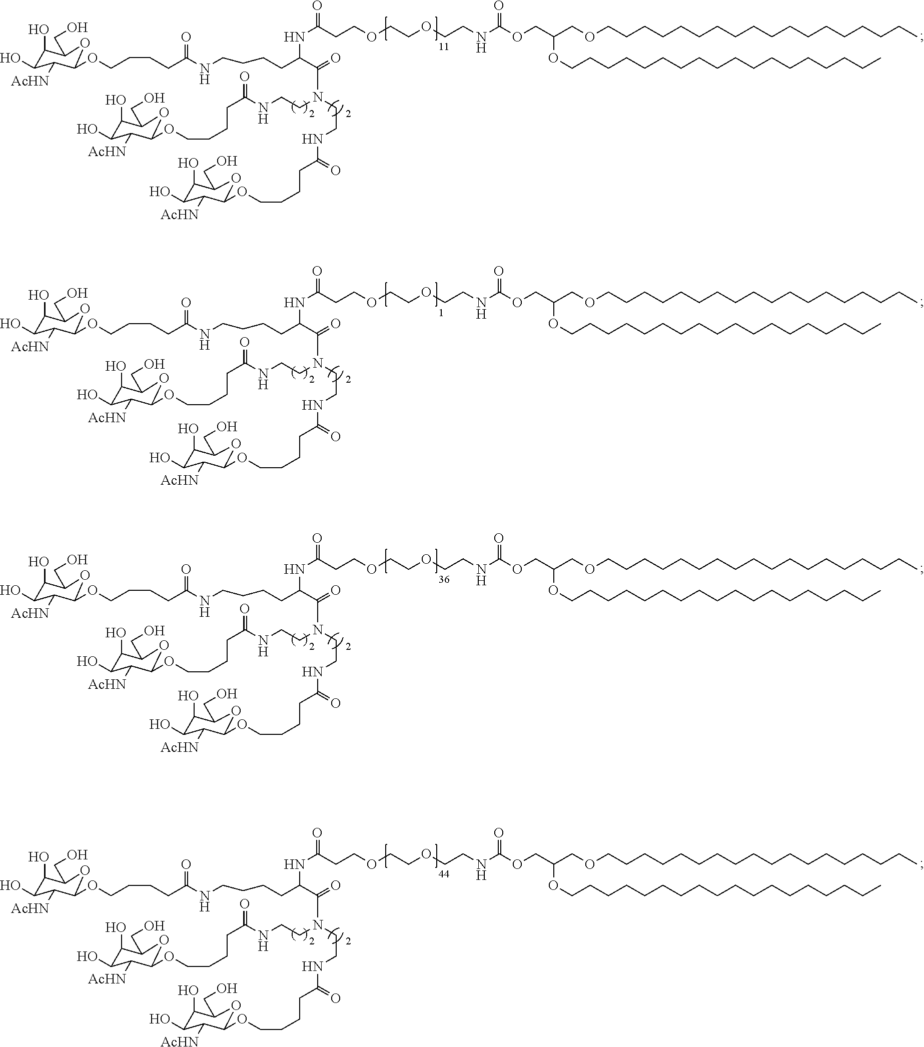

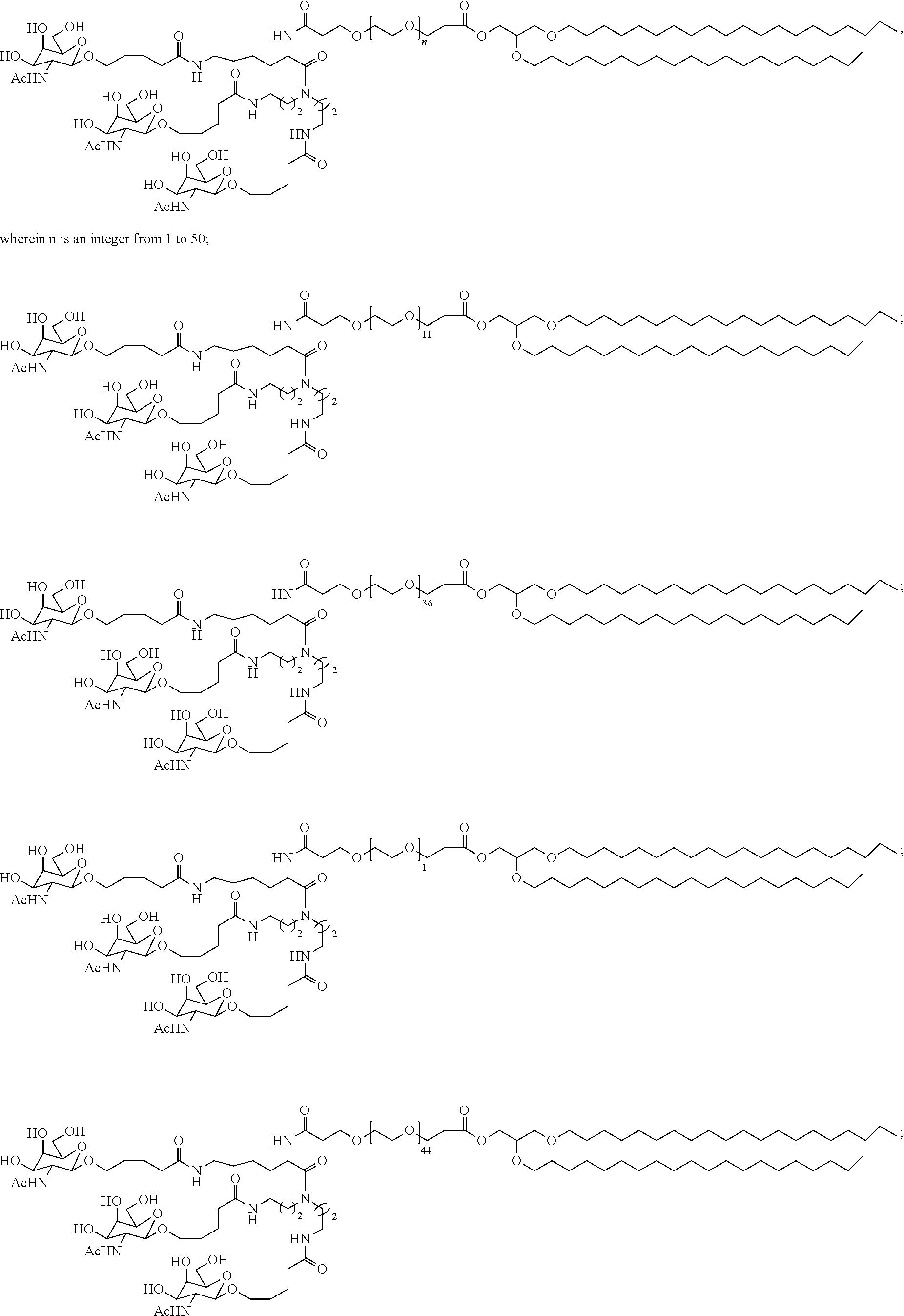

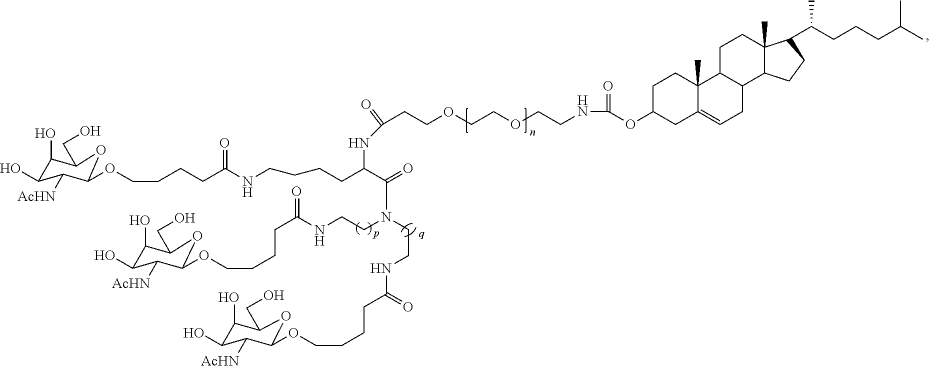

wherein each of the p and q is independently an integer from 1 to 5, and n is an integer from 1 to 50;

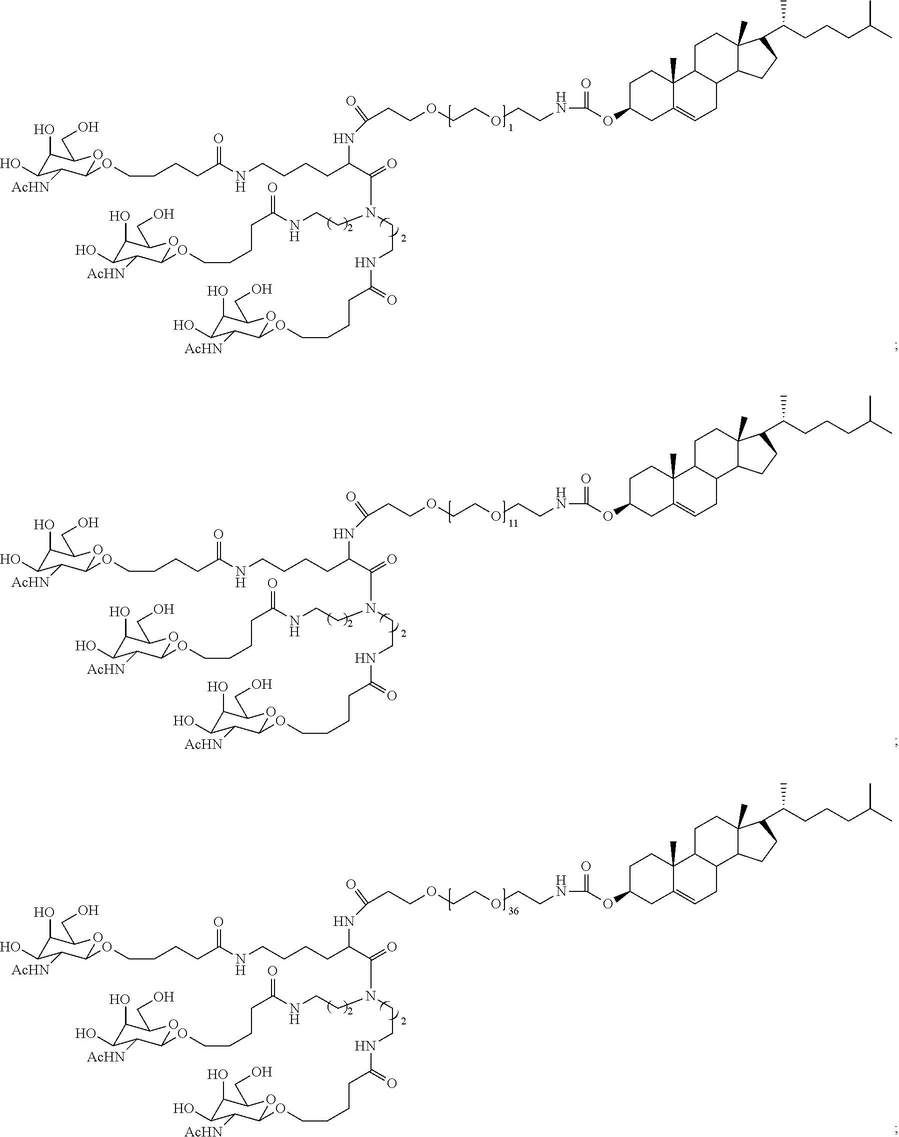

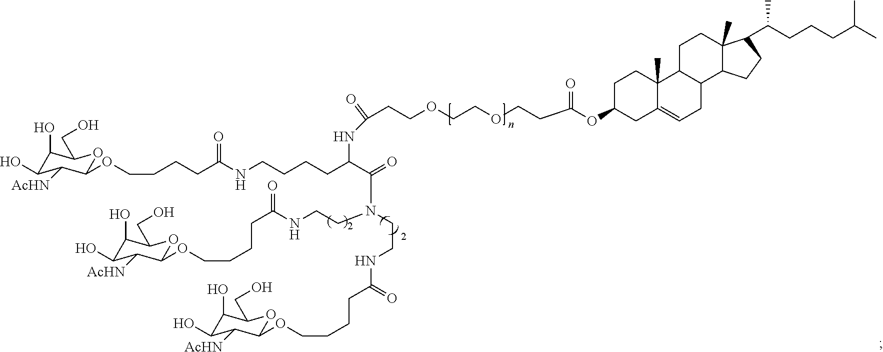

wherein n is an integer from 1 to 50;

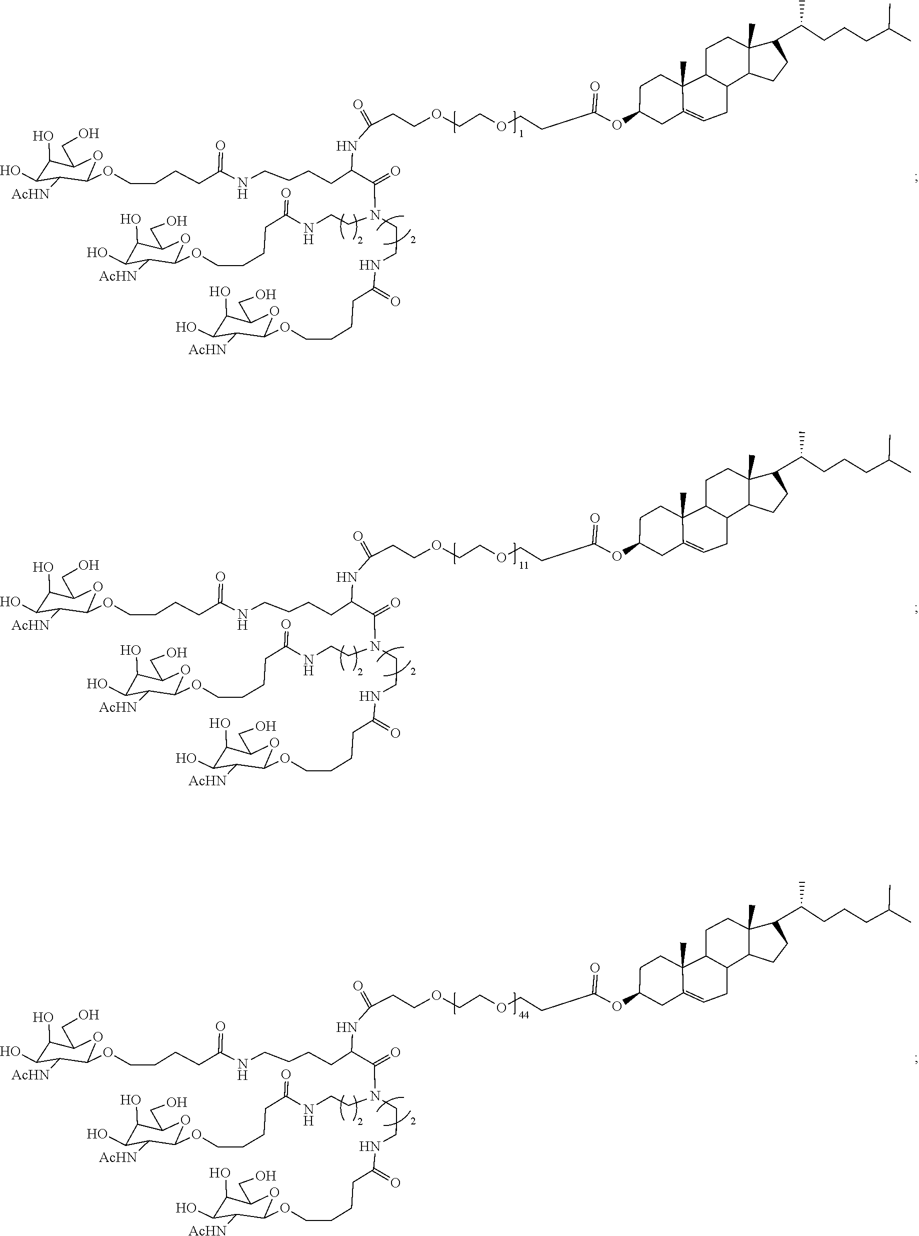

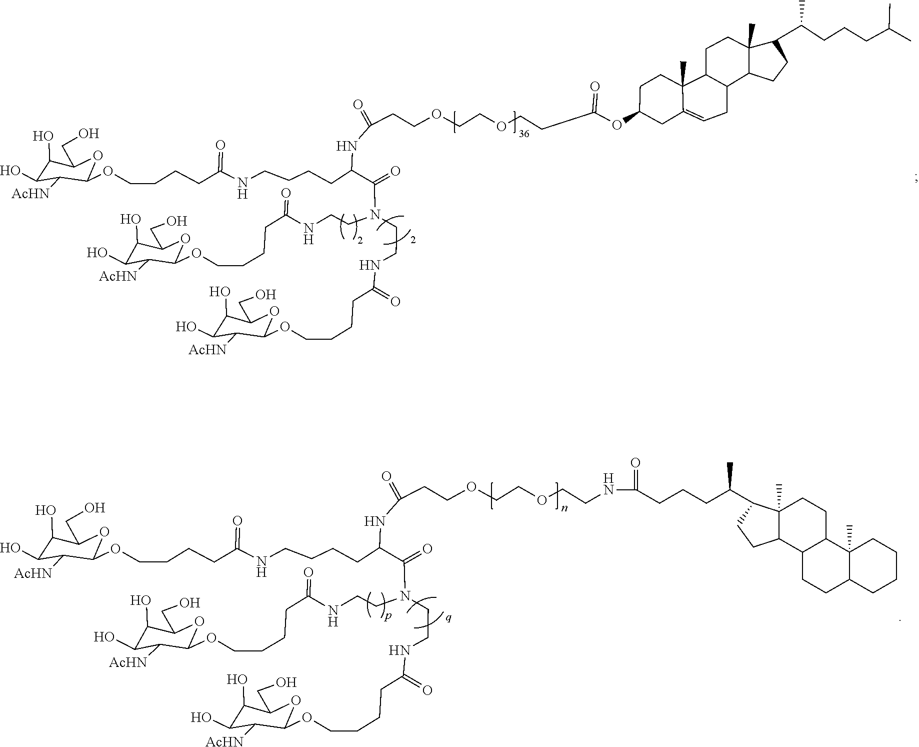

wherein each of the p and q is independently an integer from 1 to 5, and n is an integer from 1 to 50;

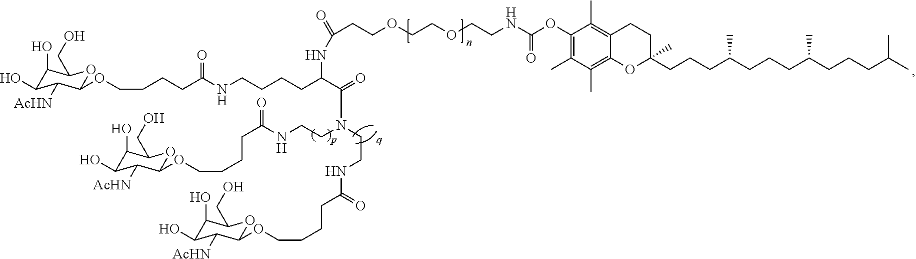

wherein each of the p and q is independently an integer from 1 to 5, and n is an integer from 1 to 50;

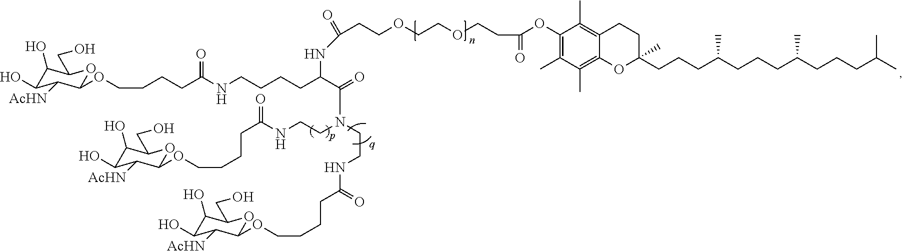

wherein each of the p and q is independently an integer from 1 to 5, and n is an integer from 1 to 50;

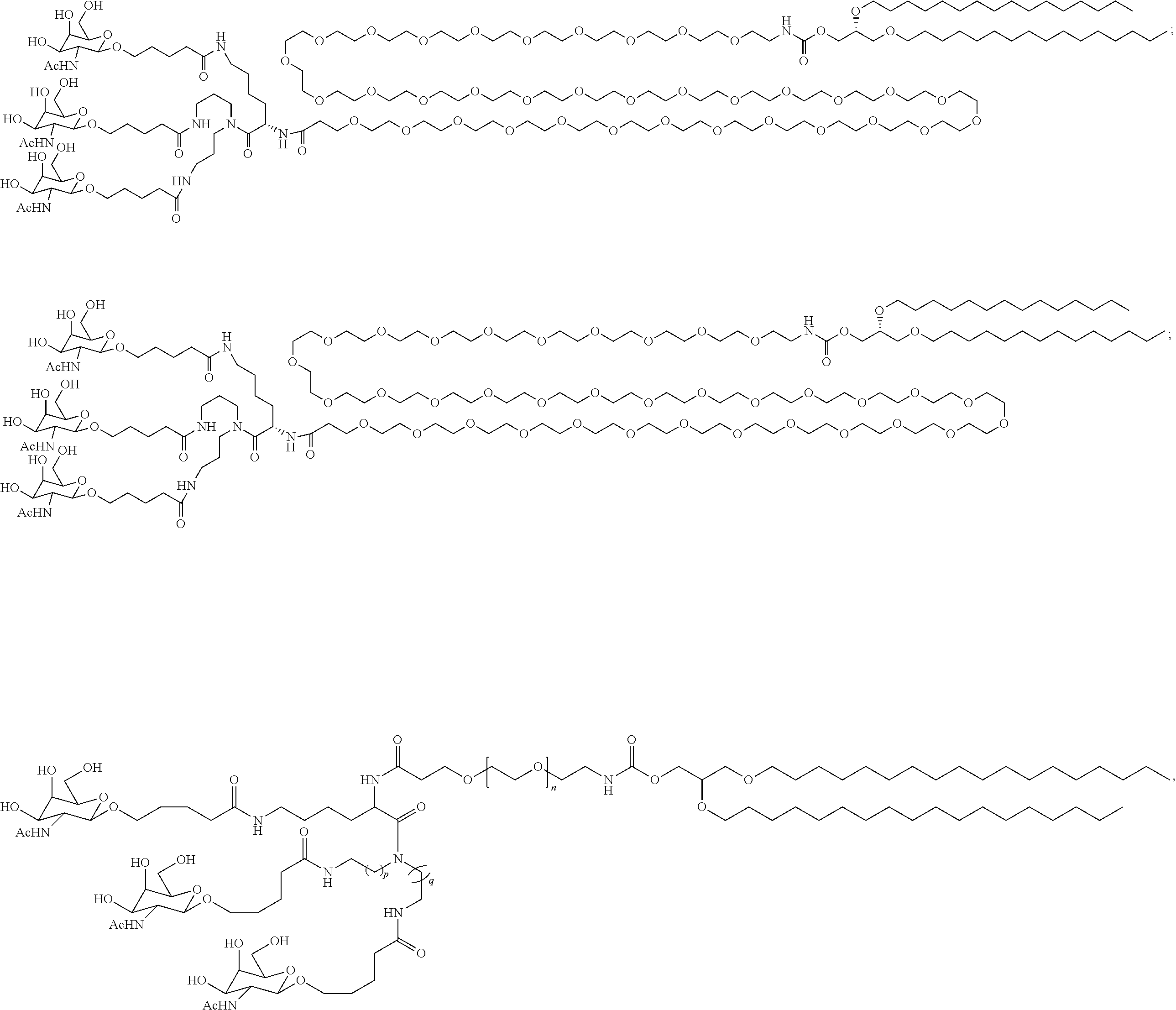

wherein each of the p and q is independently an integer from 1 to 5, and n is an integer from 33 to 39;

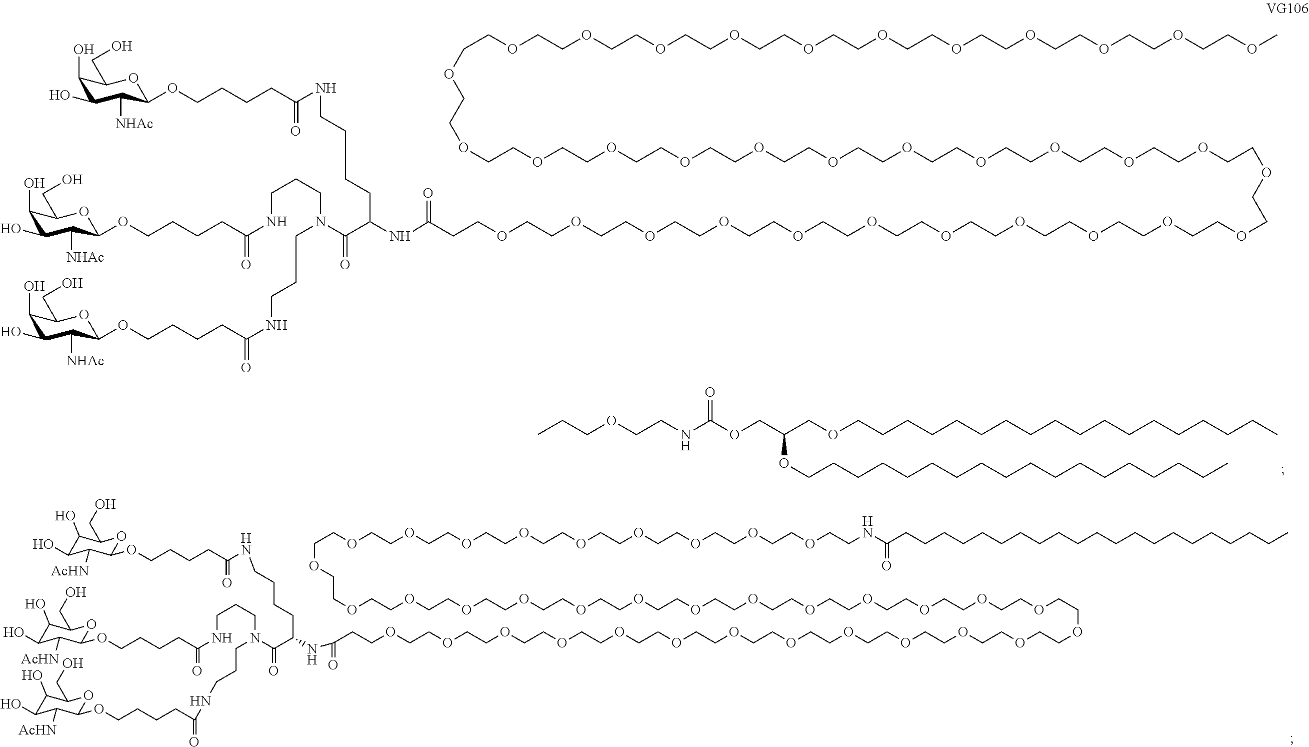

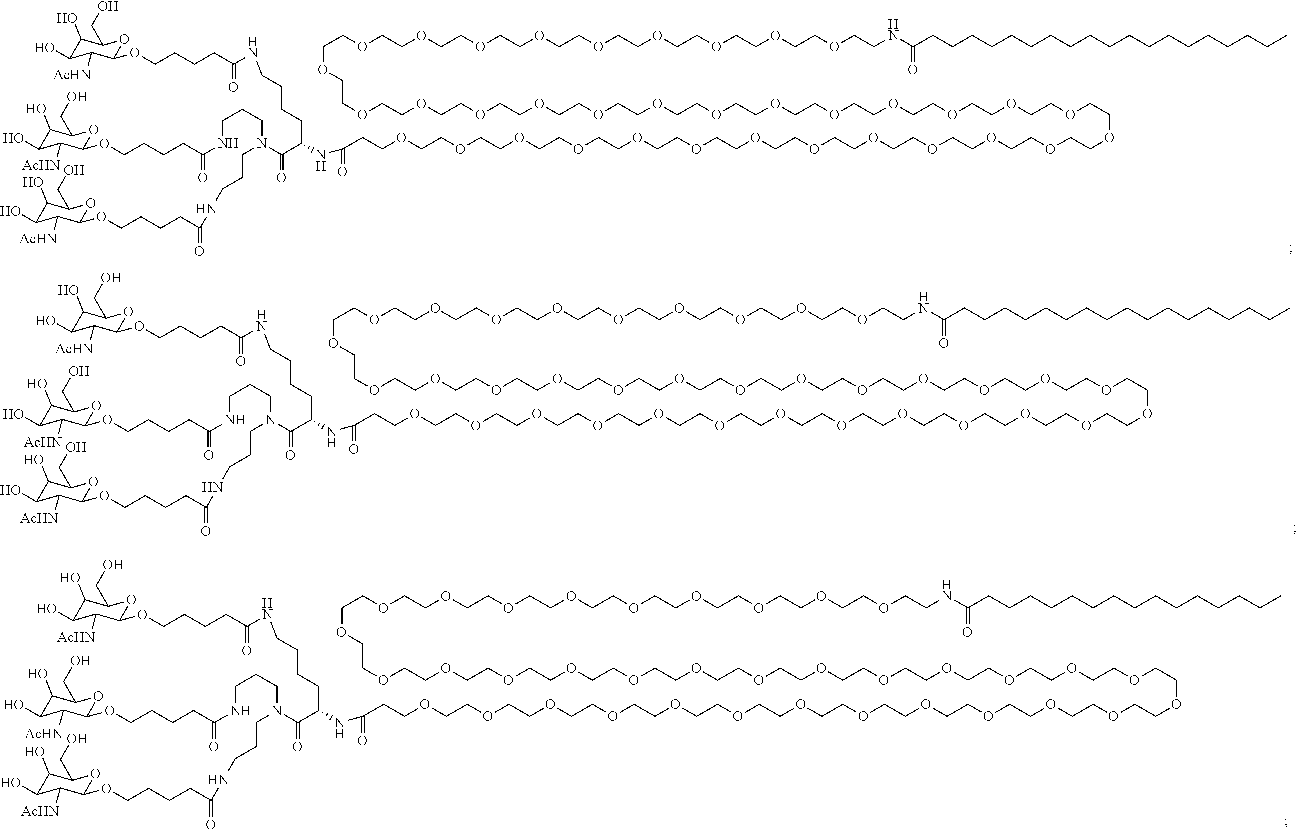

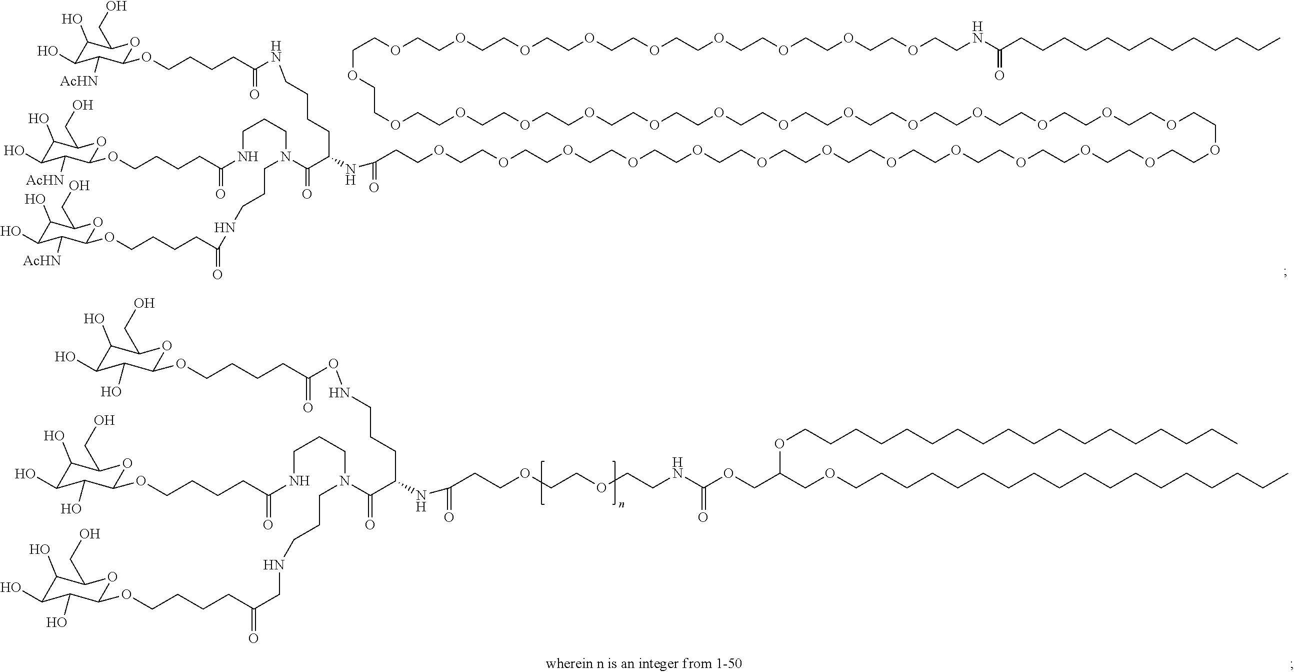

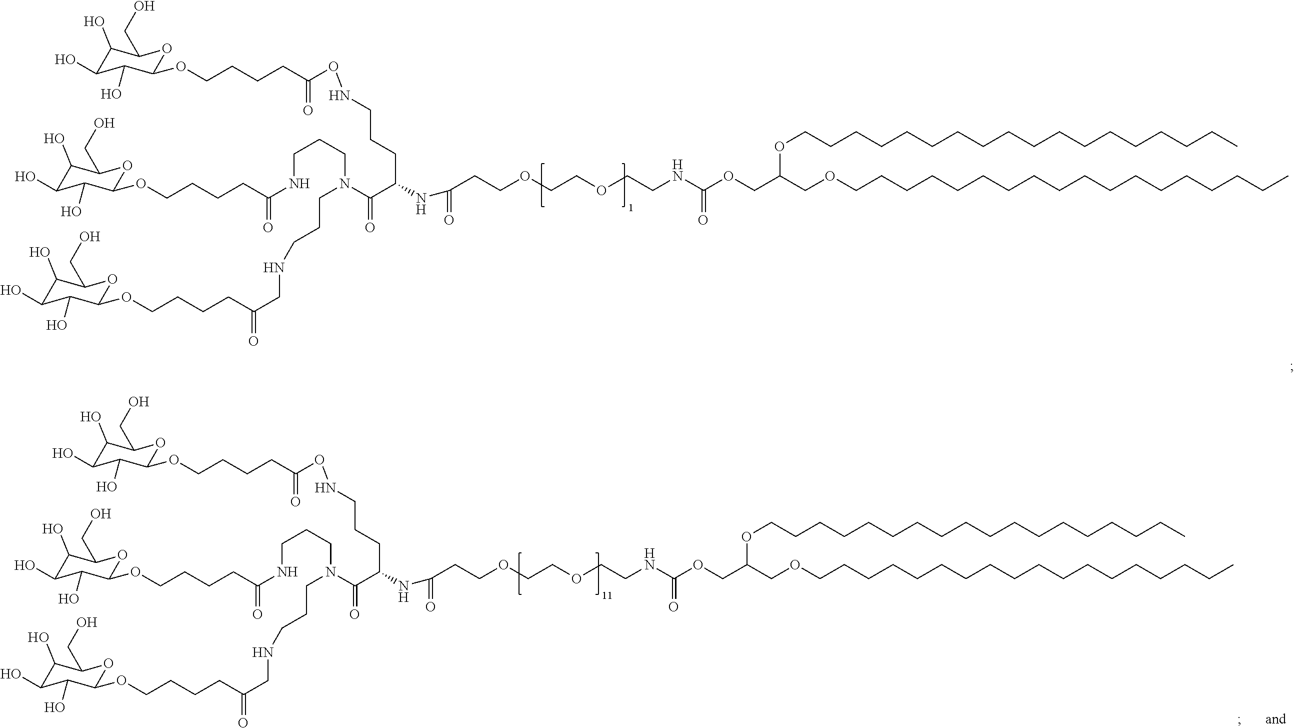

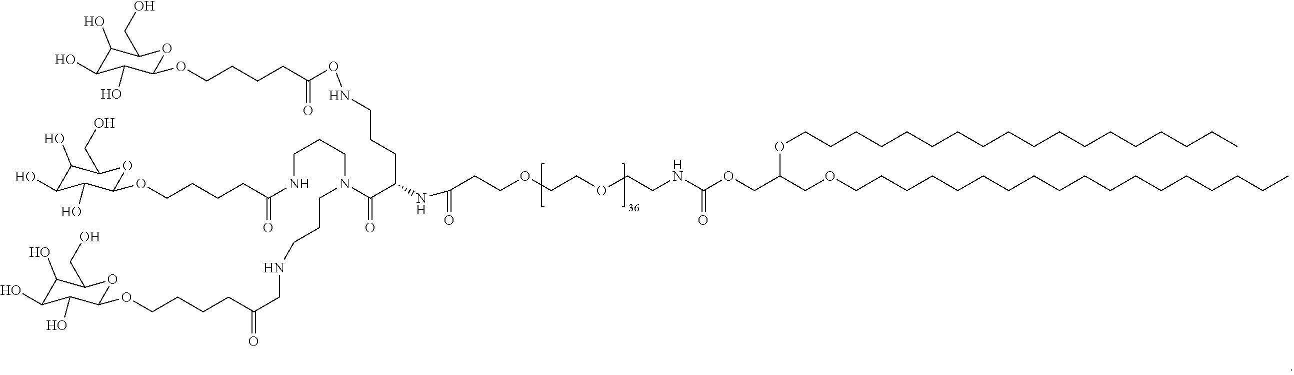

In one or more embodiments, the receptor targeting conjugate comprises from about 0.001 mol % to about 20 mol % of the total lipid content present in the nanoparticle composition.

8. Phosphate charge neutralizer

In one or more embodiments, the LNP described herein includes a phosphate charge neutralizer. In one or more embodiments, the phosphate charge neutralizer comprises arginine, asparagine, glutamine, lysine, histidine, cationic dendrimers, polyamines, or a combination thereof. In one or more embodiments, the phosphate charge neutralizer comprises one or more nitrogen atoms. In one or more embodiments, the phosphate charge neutralizer comprises a polyamine.

Suitable phosphate charge neutralizers to be used in LNP formulations, set forth below, for example include, but are not limited to, spermidine and 1,3-propanediamine.

### 9. Antioxidants

In one or more embodiments, the LNP described herein includes one or more antioxidants. In one or more embodiments, the one or more antioxidants function to reduce a degradation of the cationic lipids, the payload, or both. In one or more embodiments, the one or more antioxidants comprise a hydrophilic antioxidant. In one or more embodiments, the one or more antioxidants is a chelating agent such as ethylenediaminetetraacetic acid (EDTA) or citrate. In one or more embodiments, the one or more antioxidants comprise a lipophilic antioxidant. In one or more embodiments, the lipophilic antioxidant comprises a vitamin E isomer or a polyphenol. In one or more embodiments, the one or more antioxidants are present in the LNP composition at a concentration of at least 1 mM, at least 10 mM, at least 20 mM, at least 50 mM, or at least 100 mM. In one or more embodiments, the one or more antioxidants are present in the particle at a concentration of about 20 mM.

### 10. Other Lipids

In one or more embodiments, the disclosed LNP compositions may comprise a helper lipid. In one or more embodiments, the disclosed LNP compositions comprise a neutral lipid. In one or more embodiments, the disclosed LNP compositions comprise a stealth lipid. In one or more embodiments, the disclosed LNP compositions comprise additional lipids. Neutral lipids can function to stabilize and improve processing of the LNPs.

“Helper lipids” can refer to lipids that enhance transfection (e.g., transfection of the nanoparticle (LNP) comprising the composition as provided herein, including the biologically active agent). The mechanism by which the helper lipid enhances transfection includes enhancing particle stability. In one or more embodiments, the helper lipid enhances membrane fusogenicity. Helper lipids can include steroids, sterols, and alkyl resorcinols. Helper lipids suitable for use in the present disclosure can include, but are not limited to, cholesterol, 5-heptadecylresorcinol, and cholesterol hemisuccinate. In one or more embodiments, the helper lipid is cholesterol. In one or more embodiments, the helper lipid is cholesterol hemisuccinate.

“Stealth lipids” can refer to lipids that alter the length of time the nanoparticles can exist in vivo (e.g., in the blood). Stealth lipids can assist in the formulation process by, for example, reducing particle aggregation and controlling particle size. Stealth lipids used herein may modulate pharmacokinetic properties of the LNP. Stealth lipids suitable for use in a lipid composition of the disclosure can include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and I-Toekstra et al, Biochimica et Biophysica Acta 1660 (2004) 41-52. Additional suitable PEG lipids are disclosed, e.g., in WO 2006/007712.

In one or more embodiments, the stealth lipid is a PEG-lipid. In one embodiment, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide]. Stealth lipids can comprise a lipid moiety. In one or more embodiments, the lipid moiety of the stealth lipid may be derived from diacylglycerol or diacylglycamide, including those comprising a dialkylglycerol or dialkylglycamide group having alkyl chain length independently comprising from about C₄ to about C₄₀ saturated or unsaturated carbon atoms, wherein the chain may comprise one or more functional groups such as, for example, an amide or ester. The dialkylglycerol or dialkylglycamide group can further comprise one or more substituted alkyl groups.

The structures and properties of helper lipids, neutral lipids, stealth lipids, and/or other lipids are further described in WO2017173054A1, WO2019067999A1, US20180290965A1, US20180147298A1, US20160375134A1, U.S. Pat. Nos. 8,236,770, 8,021,686, 8,236,770B2, U.S. Pat. No. 7,371,404B2, U.S. Pat. No. 7,780,983B2, U.S. Pat. No. 7,858,117B2, US20180200186A1, US20070087045A1, WO2018119514A1, and WO2019067992A1, all of which are hereby incorporated by reference in their entirety.

### 11. LNP Formulations

Particular formulations of a nanoparticle composition comprising one or more described lipids are described herein.

The described nanoparticle compositions are capable of delivering a therapeutic agent such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic agent included in a nanoparticle composition may also be selected based on the desired delivery target or targets. For example, a therapeutic agent may be selected for a particular indication, condition, disease, or disorder and/or for delivery to a particular cell, tissue, organ, or system or group thereof (e.g., localized, or specific delivery). In certain embodiments, a nanoparticle composition may include an mRNA encoding a polypeptide of interest capable of being translated within a cell to produce the polypeptide (e.g., gene editor) of interest. Such a composition is capable of having specificity or affinity to a particular organ or cell type to facilitate drug substance delivery thereto, for example the liver or hepatocytes.

The amount of a therapeutic agent or drug substance (e.g., the mRNA that encodes for the gene editor and the guide nucleic acid, such as guide RNA) in an LNP composition may depend on the size, composition, desired target and/or application, or other properties of the nanoparticle composition. For example, the amount of an RNA in a nanoparticle composition may depend on the size, sequence, and other characteristics of the RNA. The relative amounts of a therapeutic agent and other elements (e.g., lipids) in a nanoparticle composition may also vary. In one or more embodiments, the wt/wt ratio of the lipid component to a therapeutic agent in a nanoparticle composition may be from about 5:1 to about 60:1, such as about 5:1. 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic agent may be from about 10:1 to about 40:1. In certain embodiments, the wt/wt ratio is about 20:1. The amount of a therapeutic agent in a nanoparticle composition can be measured using absorption spectroscopy (e.g., ultraviolet-visible spectroscopy).

In one or more embodiments, an LNP formulation comprises one or more nucleic acids such as RNAs. In one or more embodiments, the one or more RNAs, lipids, and amounts thereof may be selected to provide a specific N/P ratio. The N/P ratio can be selected from about 1 to about 30. The N/P ratio can be selected from about 2 to about 12. In one or more embodiments, the N/P ratio is from about 0.1 to about 50. In one or more embodiments, the N/P ratio is from about 2 to about 8. In one or more embodiments, the N/P ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. In one or more embodiments, the N/P ratio is about 2, about 2.5, about 3, about 3.5, about 4, about 4.5, about 5, about 5.5, about 6, about 6.5, about 7, about 7.5, about 8, about 9, or about 10. In one or more embodiments, the N/P ratio is from about 4 to about 6. In one or more embodiments, the N/P ratio is about 4, about 4.5, about 5, about 5.5, or about 6.

As used herein, the “N/P ratio” is the molar ratio of ionizable (e.g., in the physiological pH range) nitrogen atoms in a lipid (or lipids) to phosphate groups in a nucleic acid molecular entity (or nucleic acid molecular entities), e.g., in a nanoparticle composition comprising a lipid component and an RNA. Ionizable nitrogen atoms can include, for example, nitrogen atoms that can be protonated at about pH 1, about pH 2, about pH 3, about pH 4, about pH 4.5, about pH 5, about pH 5.5, about pH 6, about pH 6.5, about pH 7, about pH 7.5, or about pH 8 or higher. The physiological pH range can include, for example, the pH range of different cellular compartments (such as organs, tissues, and cells) and bodily fluids (such as blood, cerebrospinal fluid, gastric juice, milk, bile, saliva, tears, and urine). In certain specific embodiments, the physiological pH range refers to the pH range of blood in a mammal, for example, from about 7.35 to about 7.45. Similarly, for phosphate charge neutralizers that have one or more ionizable nitrogen atoms, the N/P ratio can refer to a molar ratio of ionizable nitrogen atoms in the phosphate charge neutralizer to the phosphate groups in a nucleic acid. In one or more embodiments, ionizable nitrogen atoms refer to those nitrogen atoms that are ionizable within a pH range between 5 and 14.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

In one or more embodiments, the LNPs are formed with an average encapsulation efficiency ranging from about 50% to about 70%, from about 70% to about 90%, or from about 90% to about 100%. In one or more embodiments, the LNPs are formed with an average encapsulation efficiency ranging from about 75% to about 98%.

In another aspect, provided herein is a lipid nanoparticle (LNP) comprising the composition as provided herein. As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In one or more embodiments, a LNP refers to any particle that has a diameter of less than 1000 nm, 500 nm, 250 nm, 200 nm, 150 nm, 100 nm, 75 nm, 50 nm, or 25 nm. In one or more embodiments, a nanoparticle may range in size from 1-1000 nm, 1-500 nm, 1-250 nm, 25-200 nm, 40-100 nm, 50-100 nm. 50-90 nm, 50-80 nm, 50-70 nm, 55-95 nm, 55-80 nm, 55-75 nm, 60-100 nm, 60-90 nm, 60-80 nm, 60-70 nm, 25-100 nm, 25-80 nm, 40-80 nm, 45-75 nm, 60-75 nm, or 60-65 nm.

In one or more embodiments, an LNP may be made from cationic, anionic, or neutral lipids. In one or more embodiments, an LNP may comprise neutral lipids, such as the fusogenic phospholipid 1,2-dioleoyl-sn-glycero-3-phosphoethanolamine (DOPE) or the membrane component cholesterol, as helper lipids to enhance transfection activity and nanoparticle stability. In one or more embodiments, an LNP may comprise hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N[1-(2,3-dioleyloxy)propyl]-N,N,N-trimethylammonium chloride), DOSPA (N,N-dimethyl-N-([2-sperminecarboxamido]ethyl)-2,3-bis(dioleyloxy)-1-propaniminium pentahydrochloride), DOTAP (1,2-Dioleoyl-3-trimethylammonium propane), DMRIE (N-(2-hydroxyethyl)-N,N-dimethyl-2,3-bis(tetradecyloxy-1-propanaminiumbromide), DC-cholesterol (3β-[N—(N′,N′-dimethylaminoethane)-carbamoyl]cholesterol), DOTAP-cholesterol, GAP-DMORIE-DPyPE, and GL67A-DOPE-DMPE (1,2-Bis(dimethylphosphino)ethane)-polyethylene glycol (PEG). Examples of cationic lipids include, but are not limited to, 98N12-5, C₁₂-200, DLin-KC2-DMA (KC2), DLin-MC3-DMA (MC3), XTC, MD1, and 7C₁. Examples of neutral lipids include, but are not limited to, DPSC, DPPC (Dipalmitoylphosphatidylcholine), POPC (1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In one or more embodiments, the lipids may be combined in any number of molar ratios to produce an LNP. In one or more embodiments, the polynucleotide may be combined with lipid(s) in a wide range of molar ratios to produce an LNP.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioallcyl, alkylsulfonyl, alkylsulfonylalkyl, arylsulfonylalkyl, alkoxy, aryloxy, aralkoxy, aminocarbonyl, alkylaminocarbonyl, aiylaminocarbonyl, alkoxycarbonyl, aryloxycarbonyl, haloalkyl, amino, trifluoromethyl, cyano, nitro, alkylamino, arylamino, alkylaminoalkyl, aiylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary substituents include amino, alkylamino, and the like.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances, one or more substituents having a double bond (e.g., “oxo” or “═O”) as the point of attachment may be described, shown, or listed herein within a substituent group, wherein the structure may only show a single bond as the point of attachment to the core structure of Formula (I). A person of ordinary skill in the art would understand that, while only a single bond is shown, a double bond is intended for those substituents.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl; likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₆ alkyl. C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In one or more embodiments, the alkyl is methyl or ethyl. In one or more embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In one or more embodiments, the alkylene is —CI-12-, —CH₂CH₂—, or —CH₂CH₂CH₂—. In one or more embodiments, the alkylene is —CH₂—. In one or more embodiments, the alkylene is —CH₂CH₂—. In one or more embodiments, the alkylene is —CH₂CH₂CH₂—.

The term “alkenyl” refers to a type of alkyl group in which at least one carbon-carbon double bond is present. In one embodiment, an alkenyl group has the formula —C(R)═CR², wherein R refers to the remaining portions of the alkenyl group, which may be the same or different. In one or more embodiments, R is H or an alkyl. In one or more embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH3, —C(CH₃)═CHCH3, and —CH₂CH═CH₂.

The term “cycloalkyl” refers to a monocyclic or polycyclic non-aromatic radical, wherein each of the atoms forming the ring (i.e., skeletal atoms) is a carbon atom. In one or more embodiments, cycloalkyls are saturated or partially unsaturated. In one or more embodiments, cycloalkyls are spirocyclic or bridged compounds. In one or more embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In one or more embodiments, the monocyclic cycloalkyl is cyclopropyl, cyclobutyl, cyclopentyl or cyclohexyl. In one or more embodiments, the monocyclic cycloalkyl is cyclopentenyl or cyclohexenyl. In one or more embodiments, the monocyclic cycloalkyl is cyclopenteny 1. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbomyl, and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “heterocycle” or “heterocyclic” refers to heteroaromatic rings (also known as heteroaryls) and heterocycloalkyls (also known as heteroalicyclic groups) that includes at least one heteroatom selected from nitrogen, oxygen, and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In one or more embodiments, heterocycles are monocyclic, bicyclic, polycyclic, spirocyclic, or bridged compounds. Non-aromatic heterocyclic groups (also known as heterocycloalkyls) include rings having 3 to 12 atoms in its ring system, and aromatic heterocyclic groups include rings having 5 to 12 atoms in its ring system. The heterocyclic groups include benzofused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl. thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, futyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furaz.anyl, benzofuraz.anyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl, or imidazol-5-yl (all C-attached). The heterocyclic groups include benzo-fused ring systems. Non-aromatic heterocycles are optionally substituted with one or two oxo (═O) moieties, such as pyrrolidin-2-one. In one or more embodiments, at least one of the two rings of a bicyclic heterocycle is aromatic. In one or more embodiments, both rings of a bicyclic heterocycle are aromatic.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heterowyl ring, the heterocycloalkyl is bonded through a non-aromatic ring atom) or bridged ring systems. The nitrogen, carbon, or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, 1,1-dioxo-thiomorpholinyl. The term heterocycloalkyl also includes all ring forms of carbohydrates, including but not limited to monosaccharides, disaccharides, and oligosaccharides. Unless otherwise noted, heterocycloalkyls have from 2 to 12 carbons in the ring. In one or more embodiments, heterocycloalkyls have from 2 to 10 carbons in the ring. In one or more embodiments, heterocycloalkyls have from 2 to 10 carbons in the ring and 1 or 2 N atoms. In one or more embodiments, heterocycloalkyls have from 2 to 10 carbons in the ring and 3 or 4 N atoms. In one or more embodiments, heterocycloalkyls have from 2 to 12 carbons, 0-2 N atoms, 0-2 O atoms, 0-2 P atoms, and 0-1 S atoms in the ring. In one or more embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted. As used herein, the term “teterocycloalkylene” can refer to a divalent heterocycloalkyl group.

The term “heteroaryl” refers to an aryl group that includes one or more ring heteroatoms selected from nitrogen, oxygen, and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In one or more embodiments, heteroaryl is pyridinyl, pyrazinyl, pyrimidinyl, thiazolyl, thienyl, thiadiazolyl, or furyl. In one or more embodiments, a heteroaryl contains 0-6 N atoms in the ring. In one or more embodiments, a heteroaryl contains 1-4 N atoms in the ring. In one or more embodiments, a heteroaryl contains 4-6 N atoms in the ring. In one or more embodiments, a heteroaryl contains 0-4 N atoms, 0-1 O atoms, 0-1 P atoms, and 0-1 S atoms in the ring. In one or more embodiments, a heteroaryl contains 1-4 N atoms, 0-1 O atoms, and 0-1 S atoms in the ring. In one or more embodiments, heteroaryl is a C₁-C₉ heteroaryl. In one or more embodiments, monocyclic heteroaryl is a C₁-C₅ heteroaryl. In one or more embodiments, monocyclic heteroaryl is a 5-membered or 6-membered heteroaryl. In one or more embodiments, a bicyclic heteroaryl is a C₆-C₉ heteroaryl. In one or more embodiments, a heteroaryl group is partially reduced to form a heterocycloalkyl group defined herein. In one or more embodiments, a heteroaryl group is fully reduced to form a heterocycloalkyl group defined herein.

As used herein, amino lipids can contain at least one primary, secondary, or tertiary amine moiety that is protonatable (or ionizable) between pH range 4 and 14. In one or more embodiments, the amine moiety/moieties function as the hydrophilic headgroup of the amino lipids. When most of the amine moiety(ies) of an amino lipid (or amino lipids) in a nucleic acid-lipid nanoparticle formulation is protonated at physiological pH, then the nanoparticles can be termed as cationic lipid nanoparticles (cLNPs). When most of the amine moiety(ies) of an amino lipid (or amino lipids) in a nucleic acid-lipid nanoparticle formulation is not protonated at physiological pH but can be protonated at acidic pH, endosomal pH for example, then the nanoparticles can be termed as ionizable lipid nanoparticles (iLNPs). The amino lipids that constitute cLNPs can be generally called cationic amino lipids (cLipids). The amino lipids that constitute iLNPs can be called ionizable amino lipids (iLipids). The amino lipid can be an iLipid or a cLipid at physiological pH.

As used herein, LNP compositions or formulations are typically sized on the order of micrometers or smaller and may include a lipid bilayer. Nanoparticle compositions encompass lipid nanoparticles (LNPs), liposomes, lipid vesicles, and lipoplexes. For example, a nanoparticle composition may be a liposome having a lipid bilayer with a diameter of 500 nm or less. The LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

As used herein, a “phospholipid” can refer to a lipid that includes a phosphate moiety and one or more carbon chains, such as unsaturated fatty acid chains. A phospholipid may include one or more multiple (e.g., double or triple) bonds. In one or more embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of an LNP to pass through the membrane, i.e., delivery of the one or more elements to a cell.

### 12. Payload

The LNPs described herein can be designed to deliver a payload, such as one or more therapeutic agent(s) or drug substances(s), to a target cell or organ of interest. In one or more embodiments, a LNP described herein encloses one or more components of a gene editor system as described herein. For example, a LNP may enclose one or more of a guide nucleic acid, such as a guide RNA, a nucleic acid encoding the guide nucleic acid, a vector encoding the guide nucleic acid, a gene editor fusion protein, a nucleic acid encoding the gene editor fusion protein, a programmable DNA binding domain, a nucleic acid encoding the programmable DNA binding domain, or all or any combination thereof. In one or more embodiments, the nucleic acid is a DNA. In one or more embodiments, the nucleic acid is an RNA, for example, an mRNA and/or a guide RNA. In one or more embodiments, the nucleic acid(s) is/are chemically modified.

In one or more embodiments, the payload comprises one or more nucleic acid(s) (i.e., one or more nucleic acid molecular entities). In one or more embodiments, the nucleic acid is a single-stranded nucleic acid. In one or more embodiments, the single-stranded nucleic acid is a DNA. In one or more embodiments, the single-stranded nucleic acid is an RNA. In one or more embodiments, the nucleic acid is a double-stranded nucleic acid. In one or more embodiments, the double-stranded nucleic acid is a DNA. In one or more embodiments, the double-stranded nucleic acid is an RNA. In one or more embodiments, the double-stranded nucleic acid is a DNA-RNA hybrid. In one or more embodiments, the nucleic acid is a messenger RNA (mRNA), a microRNA, an asymmetrical interfering RNA (aiRNA), a small hairpin RNA (shRNA), an antisense oligonucleotide, or a Dicer-Substrate dsRNA. In one or more embodiments, the single-stranded nucleic acids form secondary structure, one or more stem-loops for example. In one or more other embodiments, the single stranded nucleic acids contain one or more stem-loops and single-stranded regions within the molecule.

### 13. Exemplary LNP Formulations

The following are example formulations of LPA LNPs.

### Example Lipid Ratios (Molar Percentages)

| LNP | | | GalNAc | GalNAc |
|---|---|---|---|---|
| Compo- | Formu- | Formu- | Formu- | Formu- |
| nent | lation #1 | lation #2 | lation #1 | lation #2 |
| iLipid | 40-60 | 50 +/− 10% | 40-60 | 50 +/− 10% |
| Choles- | 28-48 | 38 +/− 10% | 27.95-47.95 | 37.95 +/− 10% |
| terol | | | | |
| DSPC | 5-13 | 9 +/− 10% | 5-13 | 9 +/− 10% |
| PEG-Lipid | 2-5 | 3 +/− 10% | 2-5 | 3 +/− 10% |
| GalNAc- | N/A | N/A | 0.02-0.09 | 0.05 +/− 10% |
| lipid | | | | |

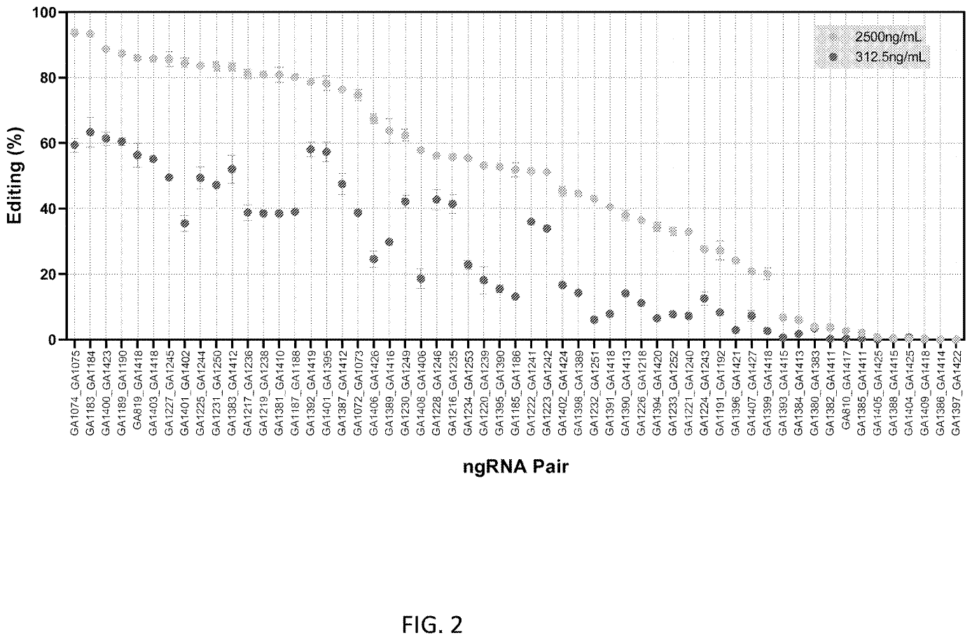

The above LNPs may be formulated with an mRNA:gRNA weight ratio of 1:1+/−5%, 10%, 15%, 20%, 30%, 40%, 50% 60%, 70%, 80%, 90%, or 100% of the mRNA or gRNA or any therapeutically effective ratio. Thus, for example an mRNA:gRNA weight ratio of 1:1+/−10% of gRNA would mean any mRNA:gRNA ratio between 1:0.9 to 1:1.1. The N/P ratio of the LNP may be from about 4 to about 7, about 4, about 4.5, about 5, about 5.5, or about 6, about 6.5, or about 7, with each ratio +/−5-20%. The LNPs may be contained in a pharmaceutically acceptable solution that may, for example, comprise a buffer having a pH of about 7.5+/−1.5 and including tris and/or sucrose. The gRNAs may comprise sequences substantially identical to those specified in Table 3. Thus, for example, one or more of the gRNA combinations set forth in FIG. 2 may comprise the LNP payload. The mean diameter of the LNPs may be about 70 nm +/−20 nm, 70 nm+/−10 nm, 70 nm+/−5 nm; 60 nm+/−20 nm, 60 nm+/−10 nm, 60 nm+/−5 nm; 50 nm+/−20 nm, 50 nm+/−10 nm, 50 nm+/−5 nm; 45 nm+/−20 nm, 45 nm+/−10 nm, 45 nm+/−5 nm.

Although LNPs are described herein as a suitable delivery system, other systems may be employed to deliver gRNA/mRNA to a cell or subject. Some such suitable systems include virus-like particle delivery systems, virus delivery systems such as AAV delivery systems, and any other delivery systems.

### VIII. KITS

It is contemplated herein that the therapeutic agents or drug substances disclosed herein are part of a kit as described herein. Accordingly, one aspect of the disclosure relates to kits including the compositions comprising guide nucleic acids, such as guide RNAs, as provided herein, the gene editor or gene editor system as provided herein, the compositions as provided herein, and/or the lipid nanoparticle formulations as provided herein for treating or preventing a condition. The kits can further include one or more additional therapeutic regimens or agents for treating or preventing a condition.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material (including use and/or disposal instructions) suitable for a selected formulation and intended mode of administration and treatment.

For example, the container(s) include a composition as described herein, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers, or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### IX. DOSING

The skilled artisan will appreciate that certain factors may influence the dosage and frequency of administration required to effectively treat a subject, including but not limited to the severity of the disease or disorder, previous treatments, the general characteristics of the subject including health, sex, weight, and/or age of the subject, and other diseases present. Moreover, treatment of a subject with a therapeutically effective amount of the compositions can include a single treatment or, preferably, can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. In one or more embodiments, dosage is daily, every other day, every three days, every four days, every five days, or every six days. In one or more embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, every 3 months, or longer. In one embodiment, the pharmaceutical composition comprising the gene editor system for effecting edits in the LPA gene, as described herein, is dosed once to a subject in need with no need for additional dosing. In another embodiment, the pharmaceutical composition comprising the gene editor system for effecting edits in the LPA gene, as described herein, is dosed twice with the second dose following sequentially after the first. In another embodiment, the pharmaceutical composition comprising the gene editor system for effecting edits in the LPA gene, as described herein, is dosed sequentially multiple times until a sufficient amount of therapeutically effective editing of the LPA gene, as described herein, is achieved, with the second dose following sequentially after the first. The progress of this therapy is easily monitored by conventional techniques and assays and may be determined by monitoring blood Lp(a) concentrations, which may be directly correlated with apo(a) concentrations or may be inversely correlated the size of the apo(a) protein. It should also be understood that the subject may be dosed with LNPs that contain different gene editor system components (e.g., mRNA encoding the gene editor in one LNP and gRNAs in another, a mixture of mRNA and gRNAs in the same LNP, or different gRNAs in different LNPs with or without mRNA).

The dosing regimen (including a composition disclosed herein) can vary over time. In one or more embodiments, it is contemplated that for an adult subject of normal weight, doses ranging from about 0.01 to 1000 mg/kg may be administered. In one or more embodiments, the dose is between 1 to 200 mg/kg of subject body weight. In one or more embodiments the dosing may be between 0.03 mg/kg to 3 mg/kg, 0.1 to 2 mg/kg, 0.5 to 1.5 mg/kg, or anywhere between any of the foregoing ranges. The particular dosage regimen, i.e., dose, timing, and repetition, will depend on the particular subject and that subject's medical history, as well as the properties of the polypeptide or the polynucleotide (such as the half-life of the polypeptide or the polynucleotide, and other considerations well known in the art).

The appropriate therapeutic dosage of a composition as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the therapy, and the discretion of the attending physician. Typically, the clinician may administer a therapeutic agent until a dosage is reached that achieves the desired result.

Administration of one or more compositions can be continuous or intermittent, depending, for example, upon the recipient's physiological condition, whether the purpose of the administration is therapeutic or preventative, and other factors known to skilled practitioners. The administration of a composition may be essentially continuous over a preselected period of time or may be in a series of spaced doses, e.g., either before, during, or after developing a disease.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

### X. MEANS

Described herein, among other things, are means for editing the LPA gene, means for inactivating an LPA gene, and means for treating a disease associated with the LPA gene, such atherosclerotic cardiovascular disease (ASCVD) and/or calcific aortic valve disease, in a subject in need thereof. In some embodiments, the means for editing the LPA gene, inactivating the LPA gene, or treating a disease include a gene editor system as described herein. In some embodiments, the means for editing the LPA gene, inactivating the LPA gene, or treating a disease include a delivery system comprising gene editor system as described herein. In some embodiments, the means for editing the LPA gene, inactivating the LPA gene, or treating a disease include a composition comprising gene editor system as described herein. In some embodiments, the means for editing the LPA gene, inactivating the LPA gene, or treating a disease include a pharmaceutical composition comprising gene editor system as described herein. In some embodiments, the means for editing the LPA gene, inactivating the LPA gene, or treating a disease include a lipid nanoparticle comprising gene editor system as described herein. A delivery system, composition, or pharmaceutical composition may comprise the lipid nanoparticle.

The present invention is illustrated by the following examples. It is to be understood that the particular examples, materials, amounts, and procedures are to be interpreted broadly in accordance with the scope and spirit of the invention as set forth herein.

### XI. EXAMPLES

### Example 1

### Bioinformatic Analysis

Bioinformatic analysis included information regarding MIT specificity score calculated in the CRISPOR implementation (http://crispor.tefor.net/). This computational analysis is intended to predict the specificity of the gRNA; the higher the score, the greater the predicted specificity. MIT scores for some spacer/protospacer sequences are provided in Tables 2 and 5.

### Example 2

### Evaluation of Efficiency of Nickase-Based Editing Using SpCas9-D10A and Guide/Template RNAs Via Transfection

With this example, several guide RNAs targeting the LPA gene were designed, paired based on proximity to one another, synthetically manufactured/prepared, and transfected into primary human hepatocyte cells to determine whether they were capable of editing and their respective editing efficiency.

### LPA Gene Guides

Examples of protospacer sequences to which gRNAs may have corresponding spacer sequences are shown in Table 2.

Table 7A (provided above in the Detailed Description) summarizes dose response curves in human hepatocellular carcinoma immortalized cells (HuH-7) for five (5) pairs of first and second guide RNAs using a gene editing system that comprises a dual nickase Cas9 system. The sequences of the guide oligonucleotides are provided in Table 4. The Cas9 nickase is encoded within an mRNA (MS029) that was transfected into the HuH-7 cells in a 1:1 total mRNA: total gRNA weight ratio.

Additional guide oligonucleotide pairs were tested with a similar experimental design. The results are presented in Table 7B (provided above in the Detailed Description).

### RNA Preparation

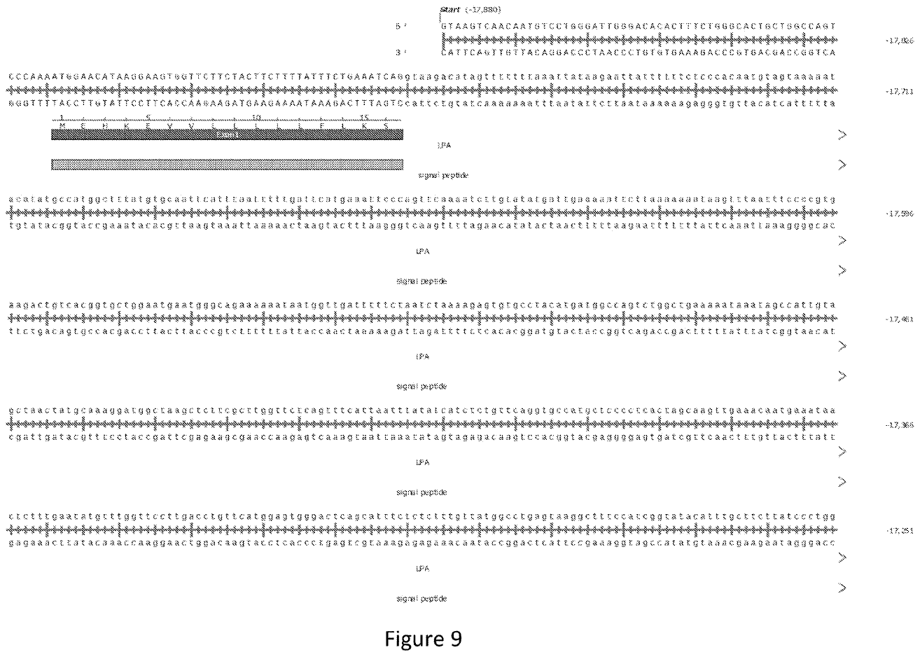

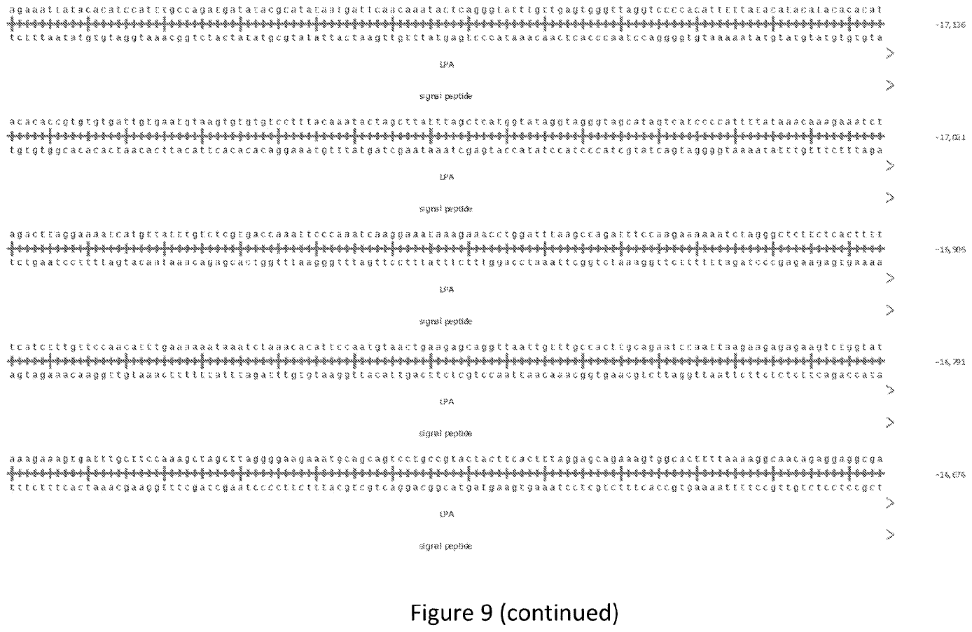

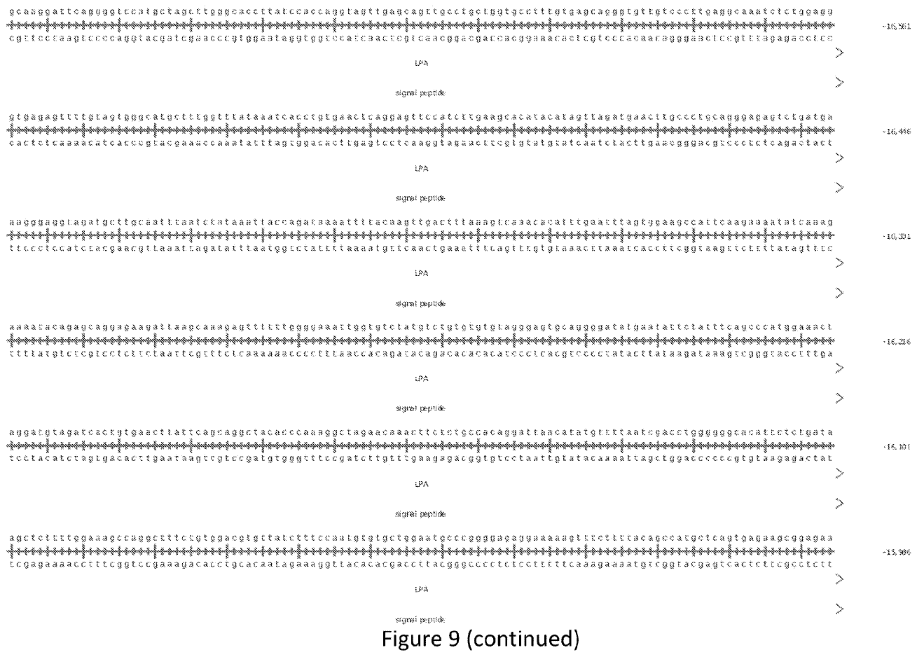

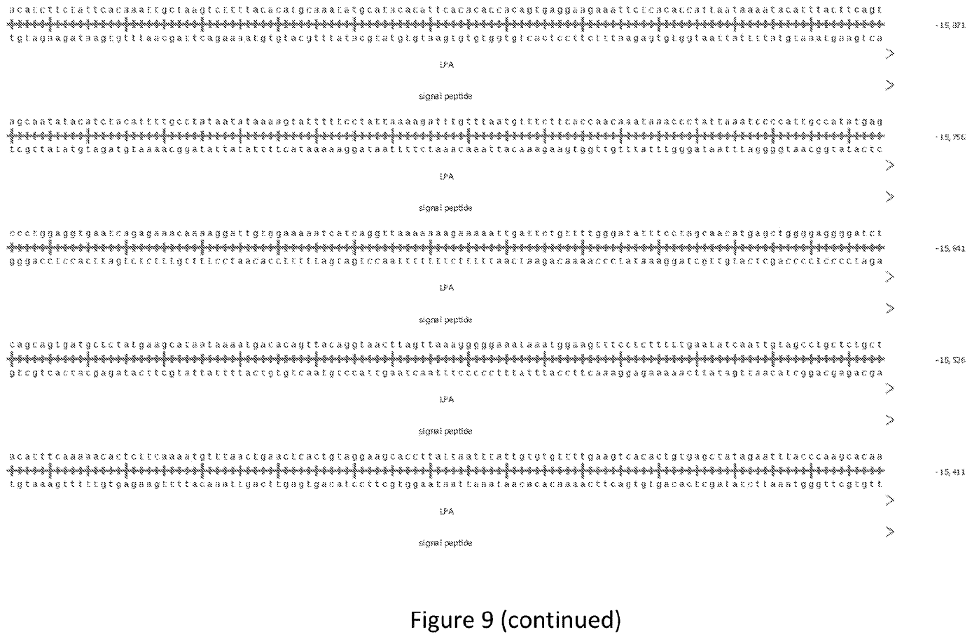

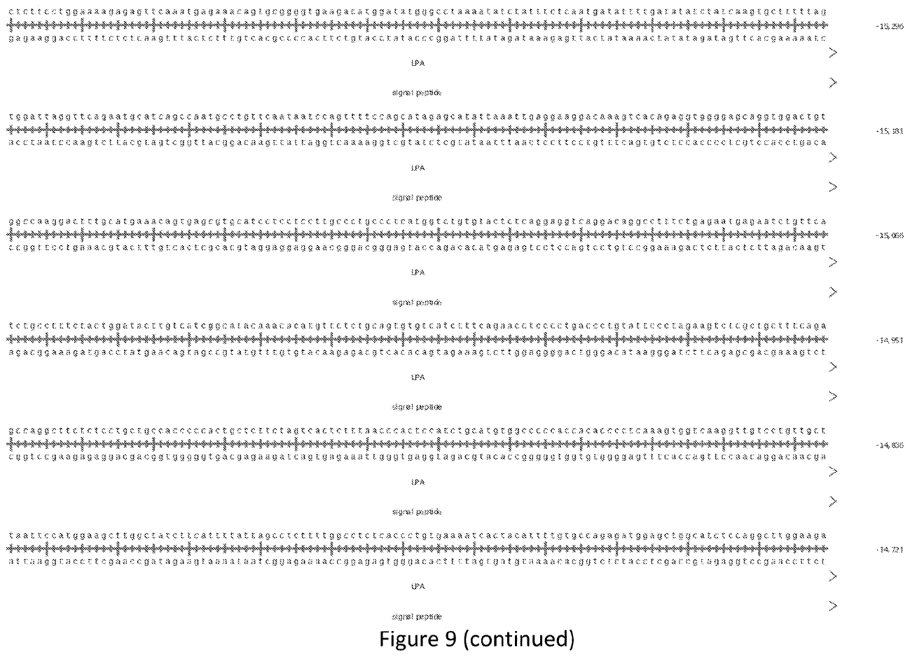

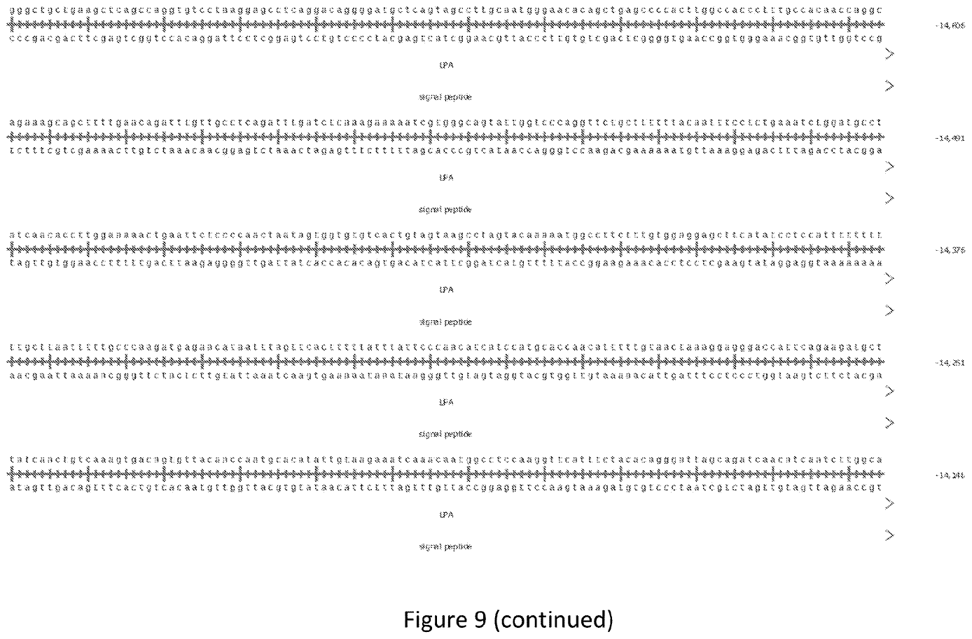

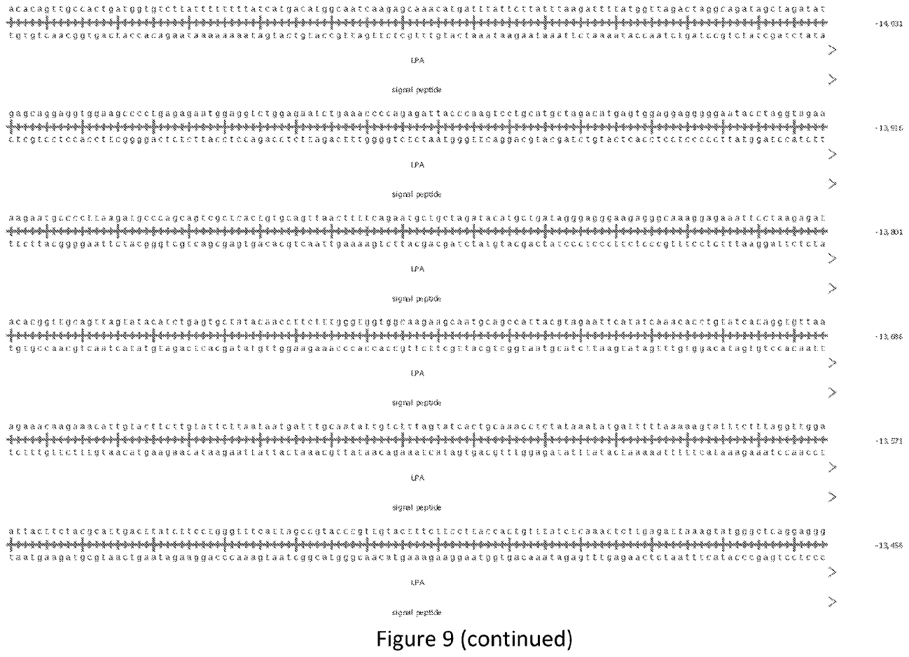

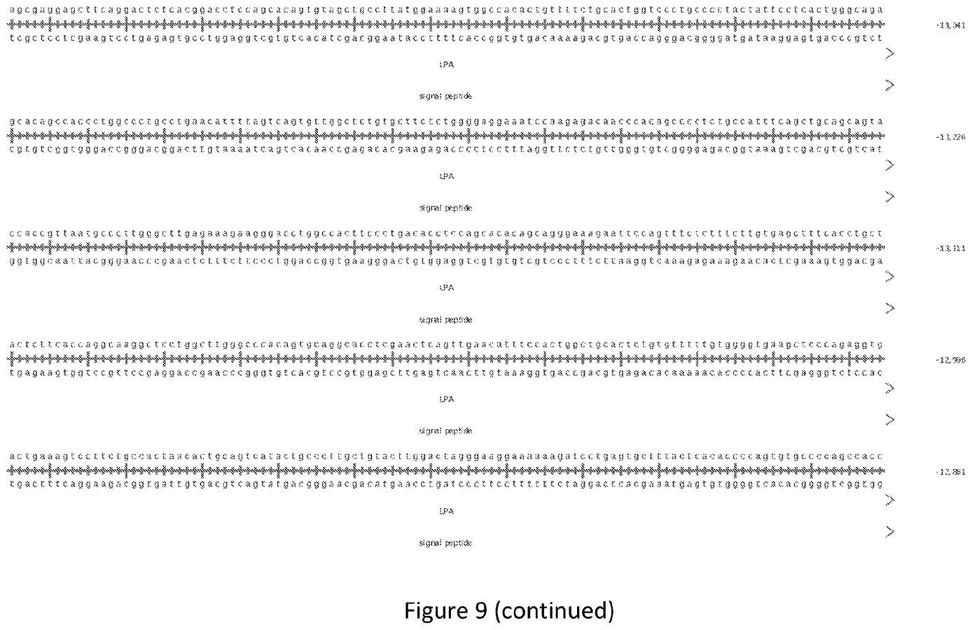

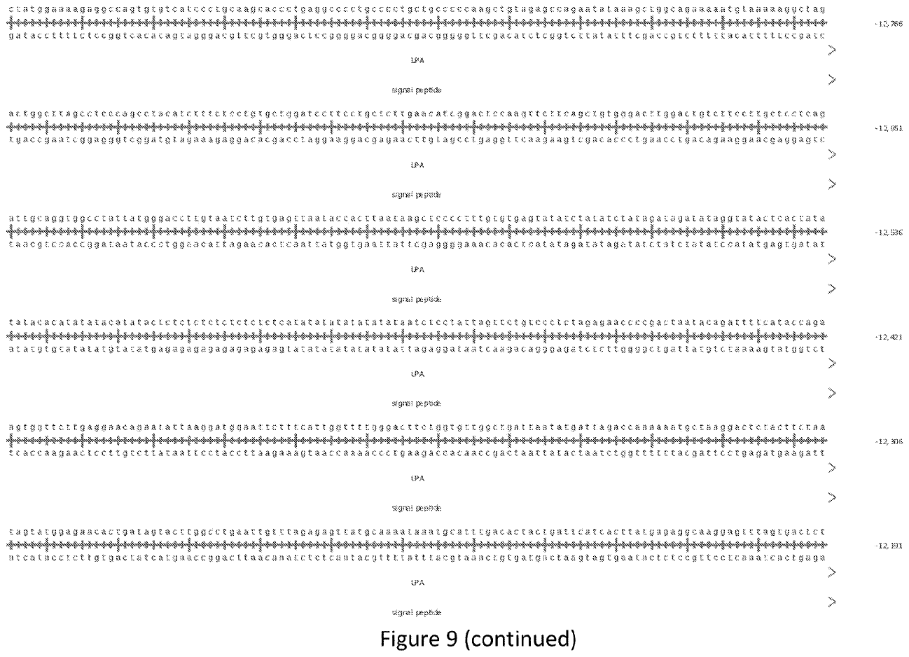

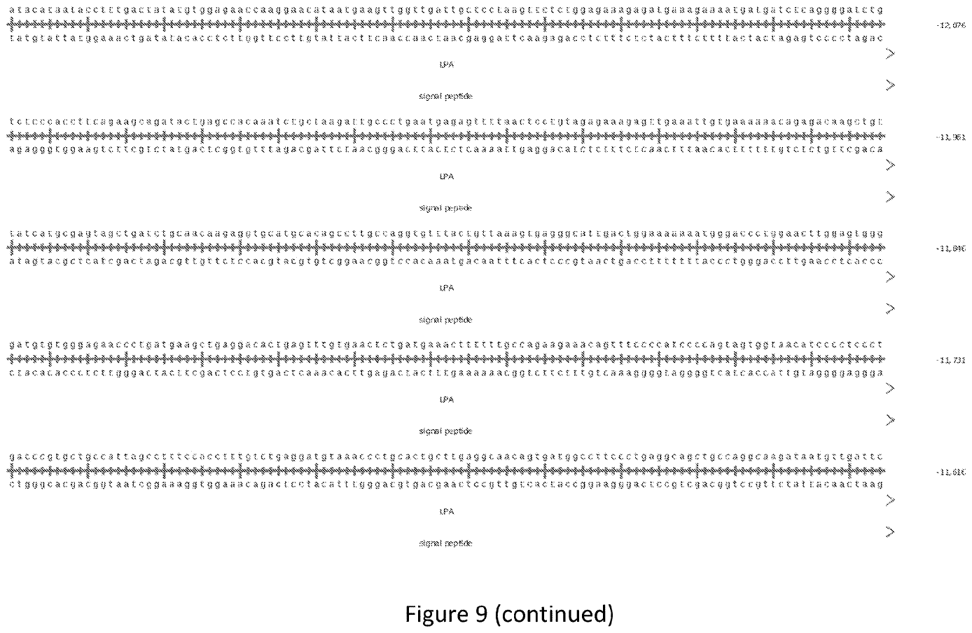

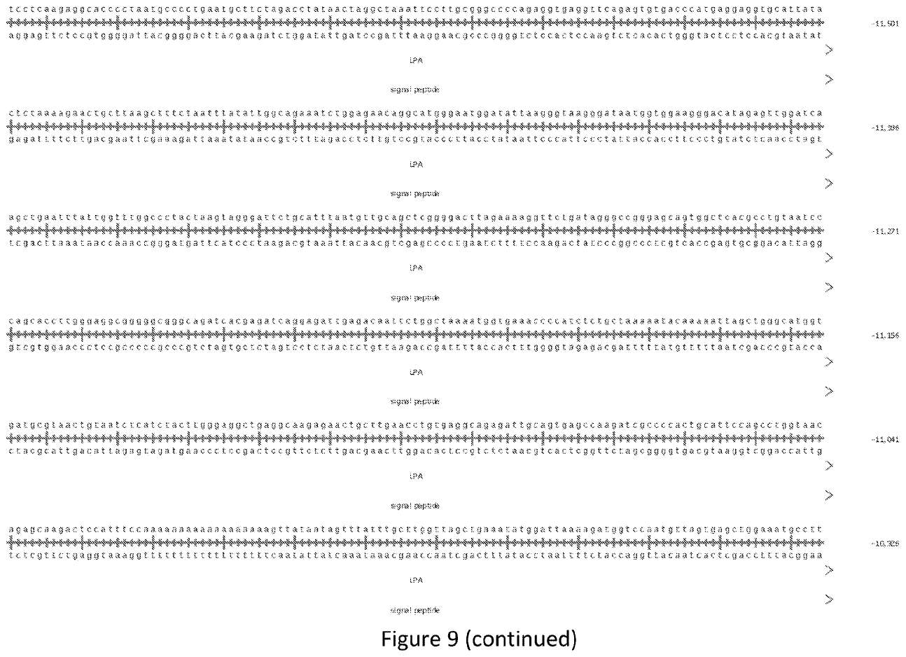

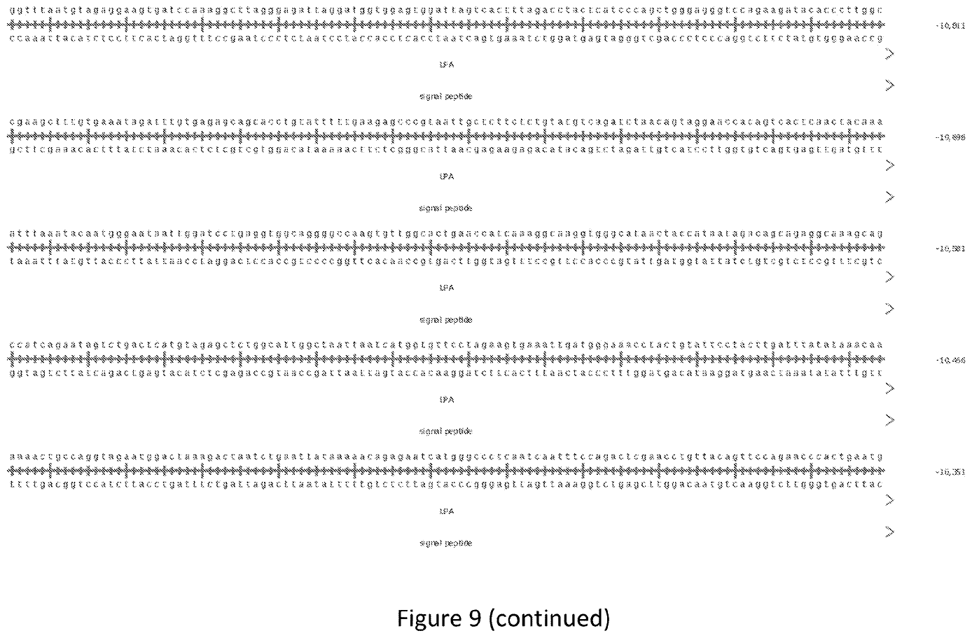

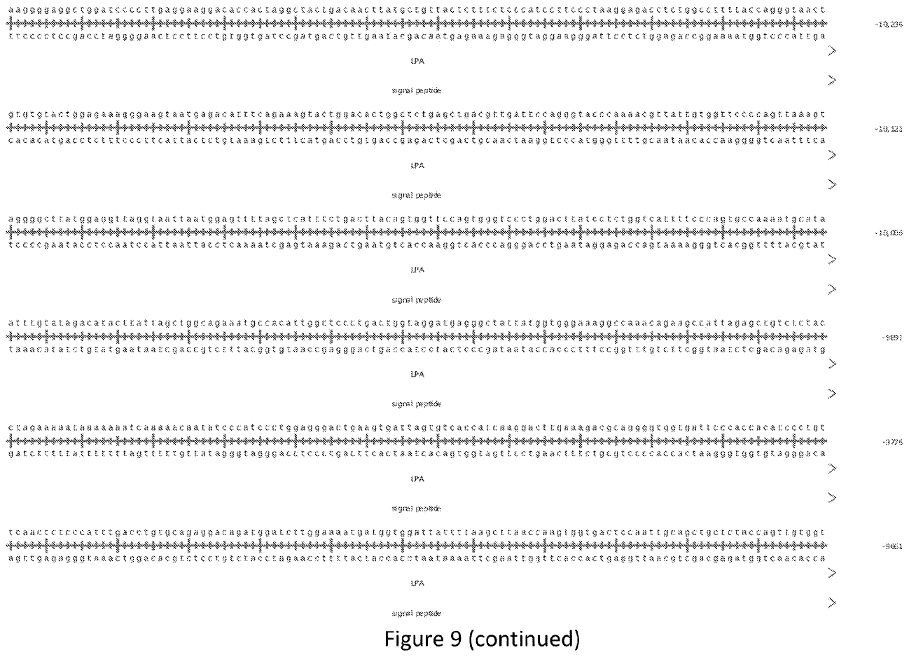

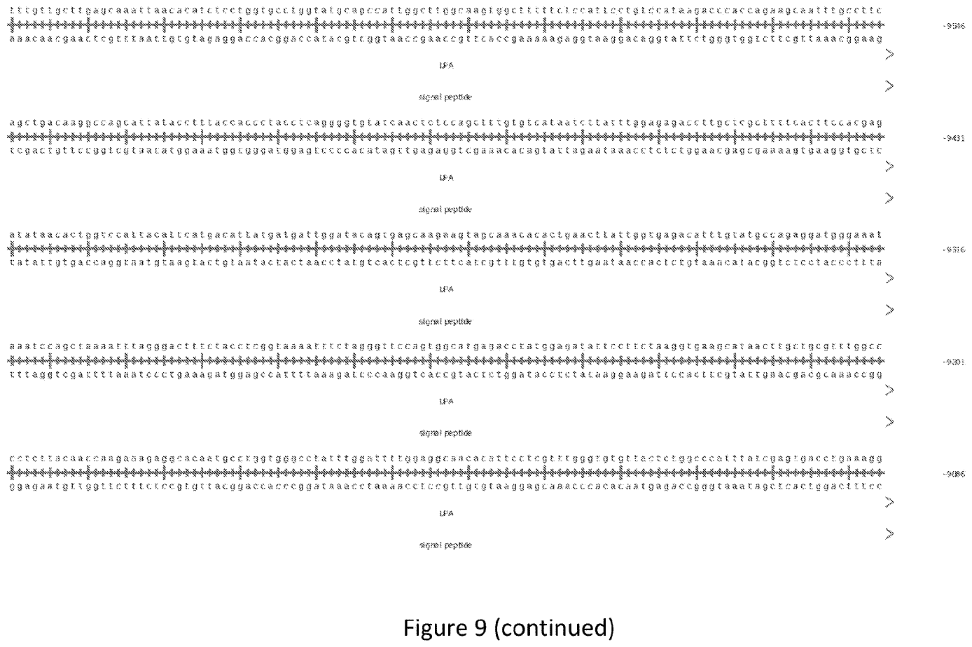

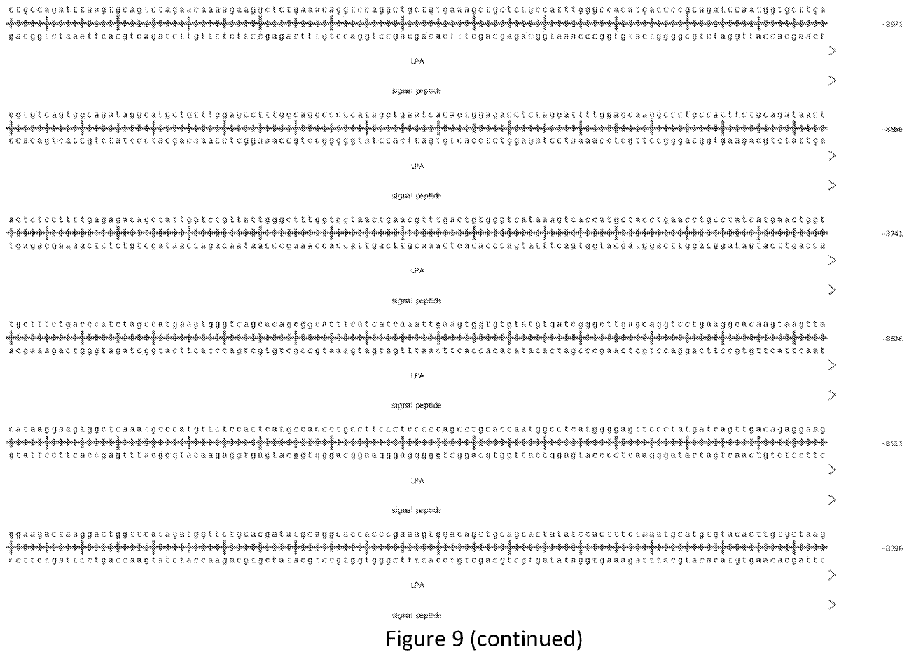

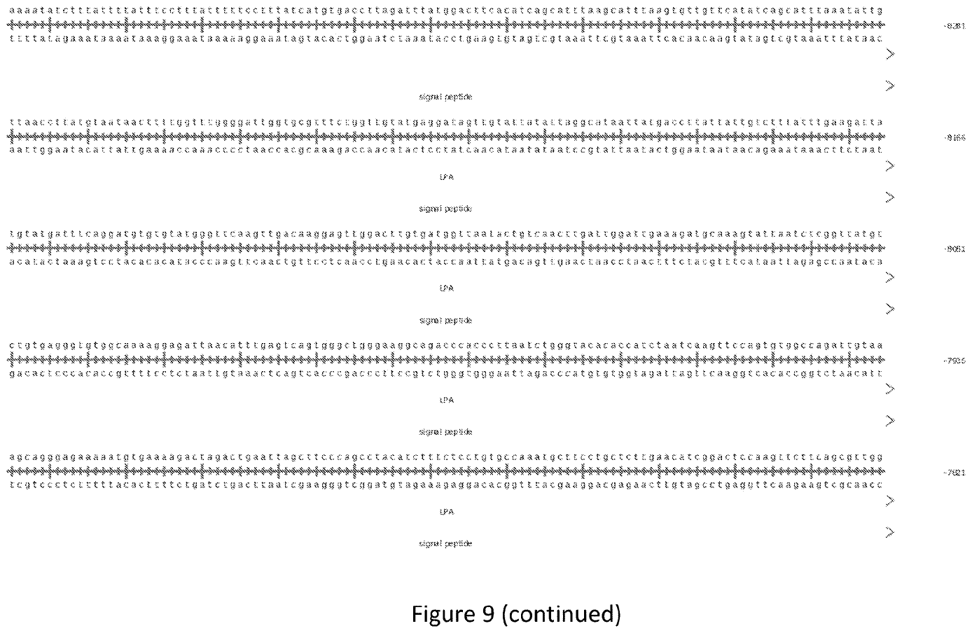

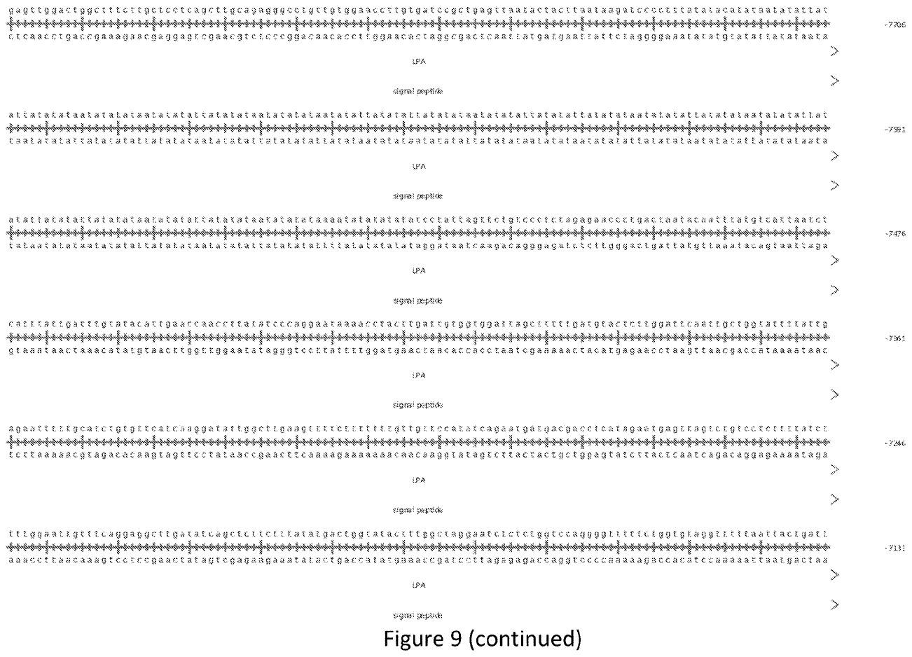

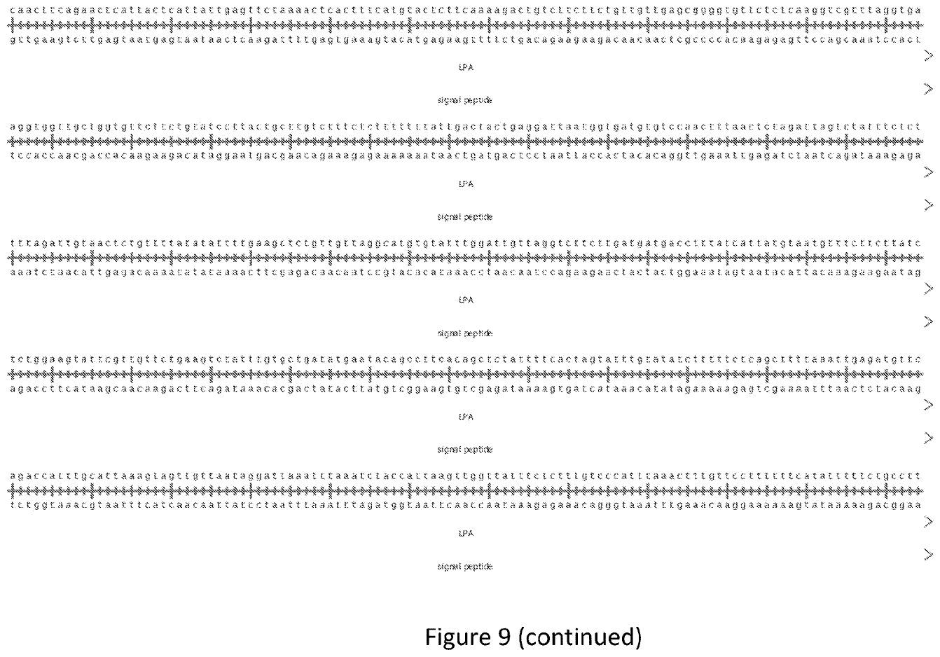

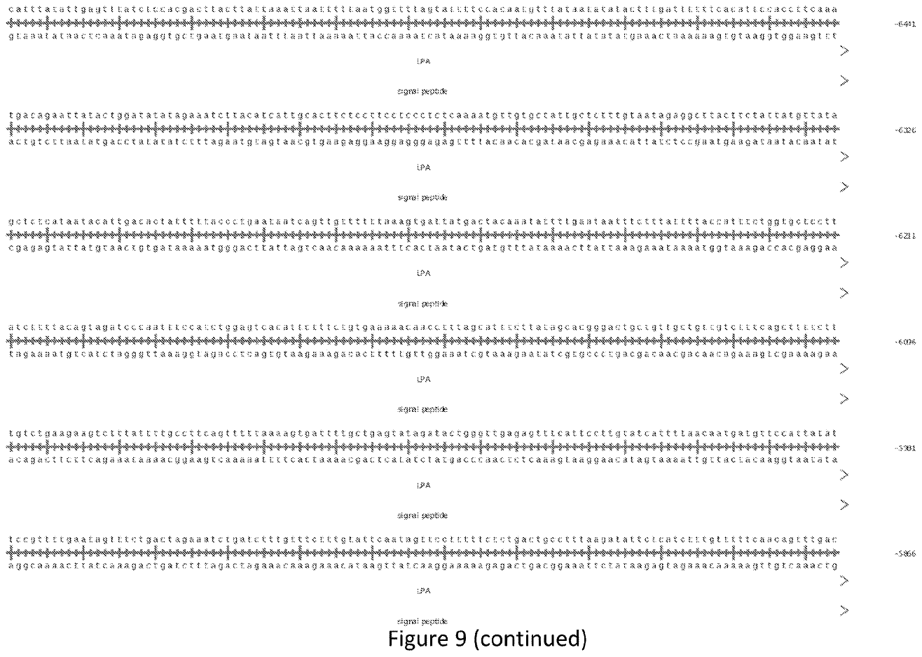

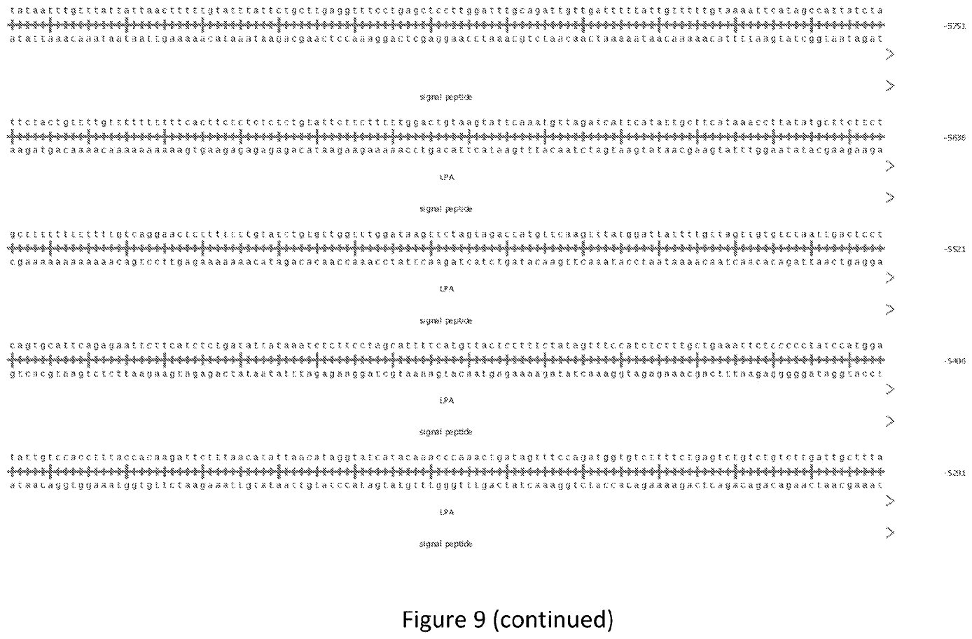

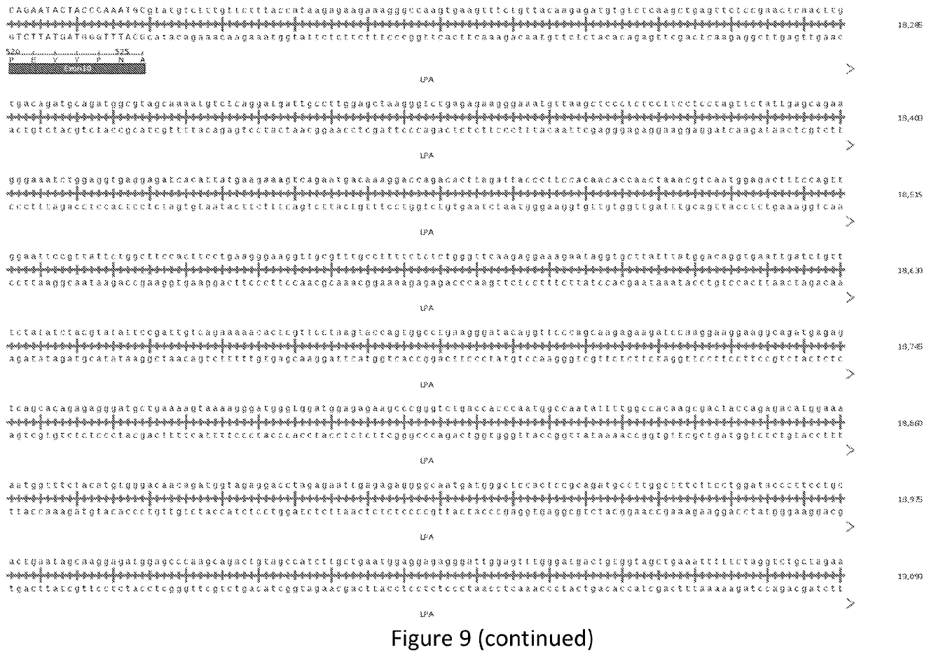

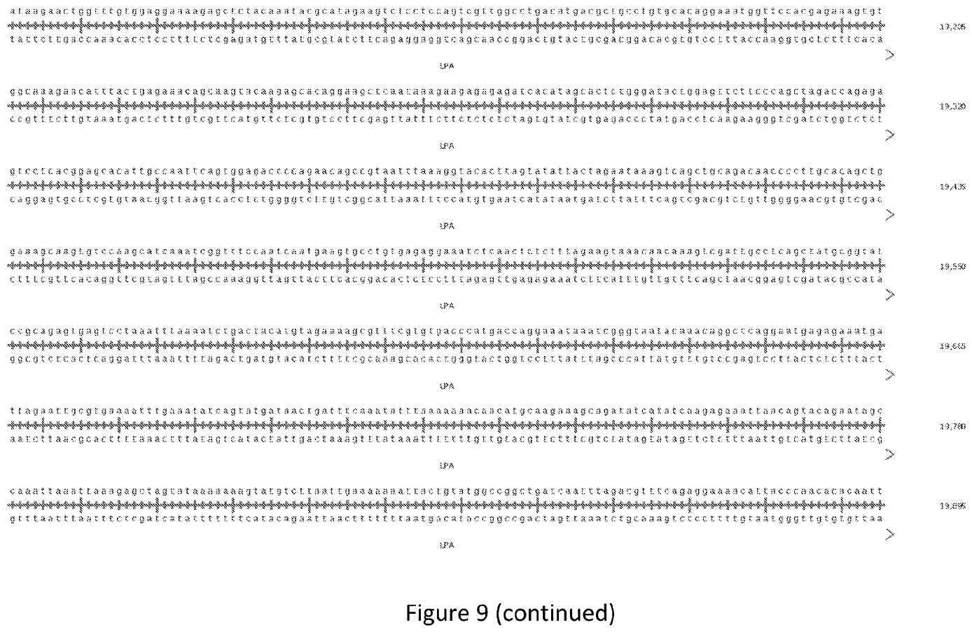

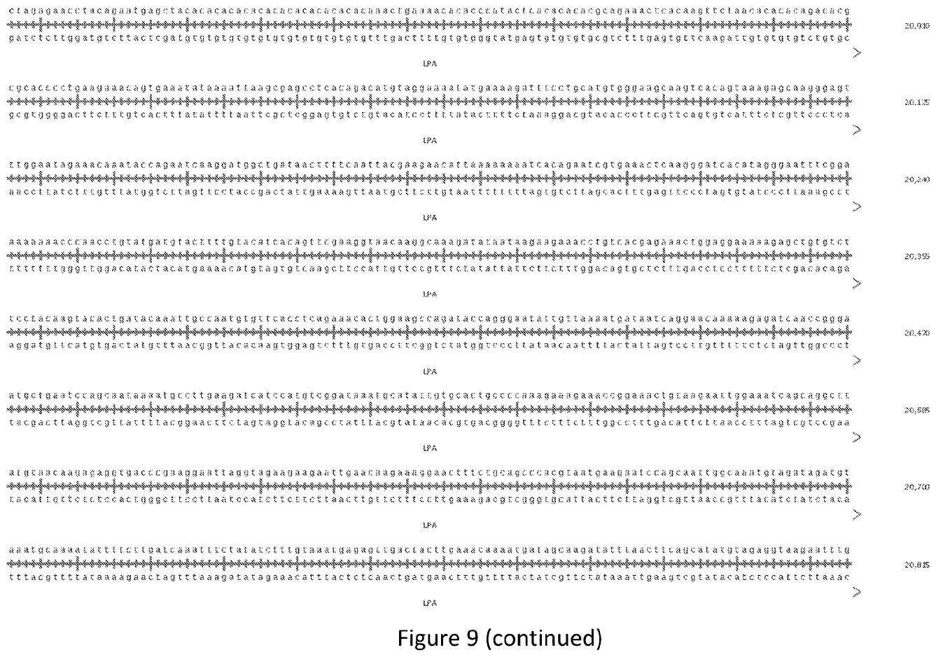

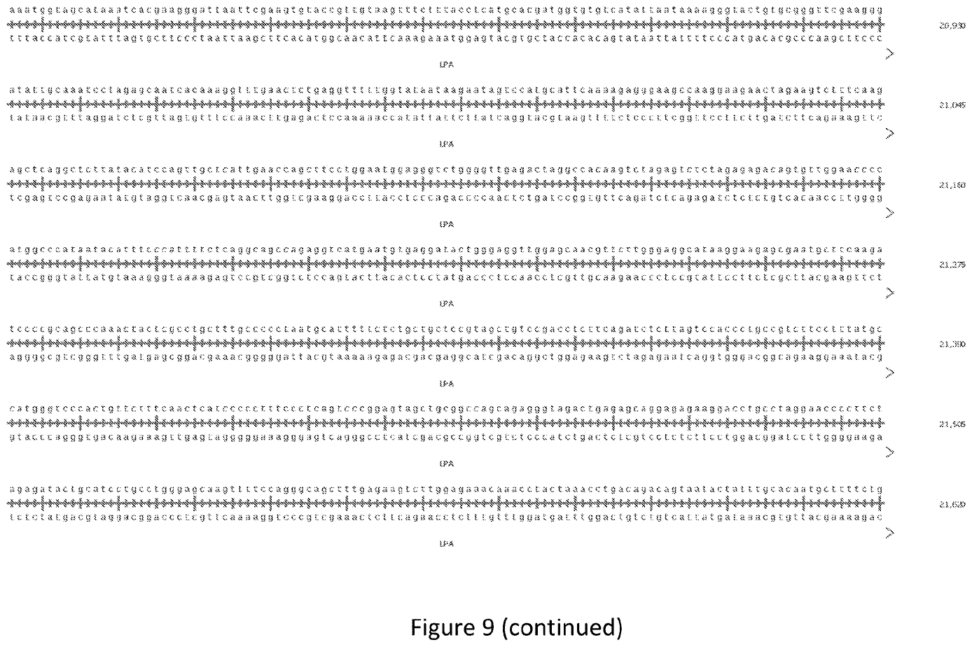

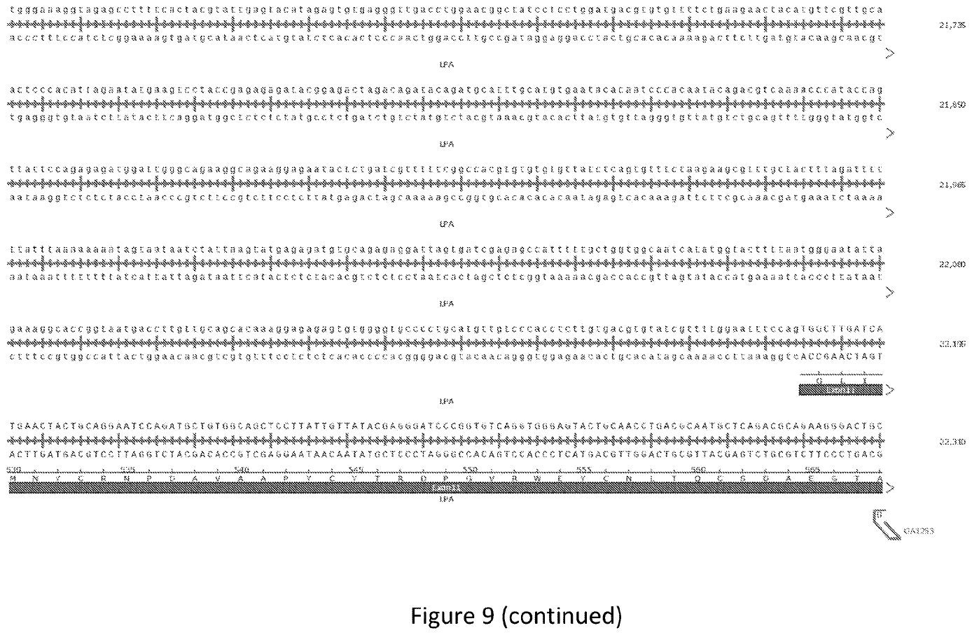

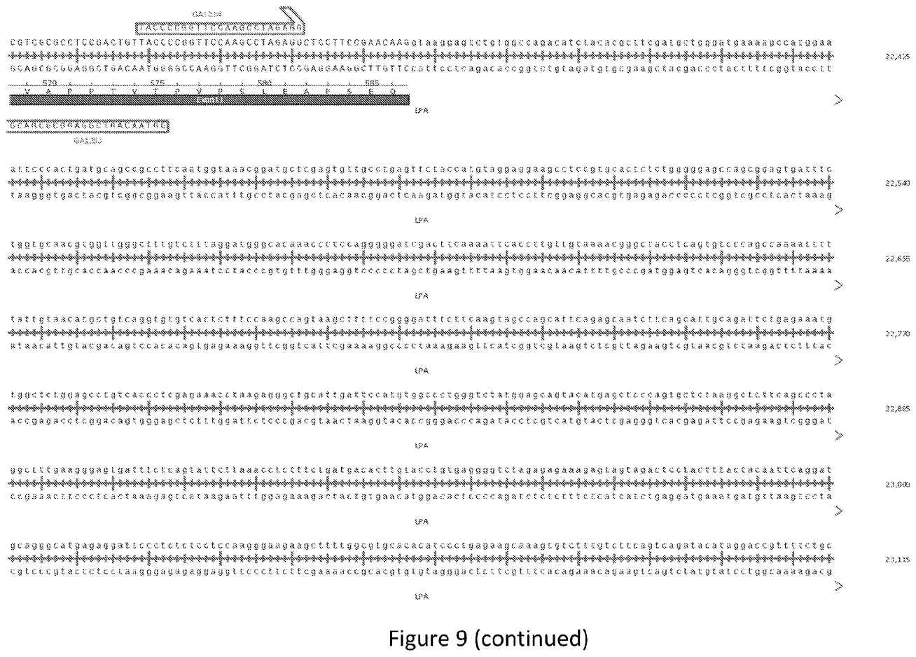

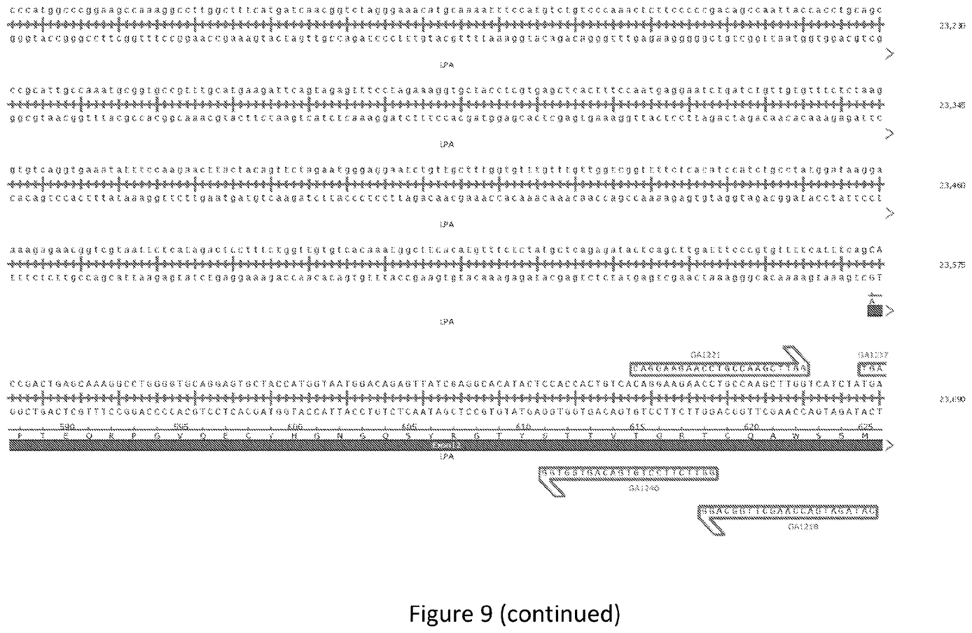

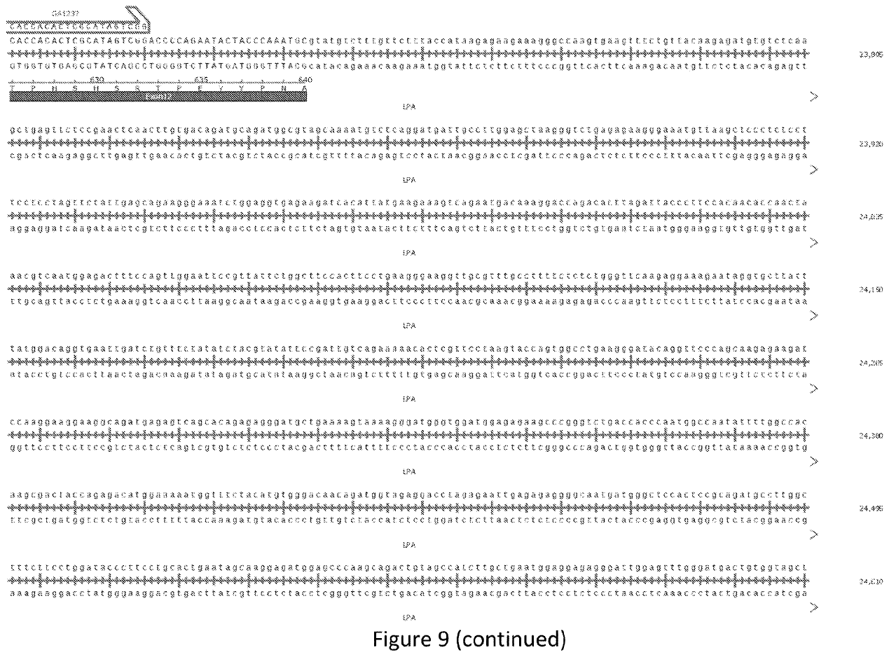

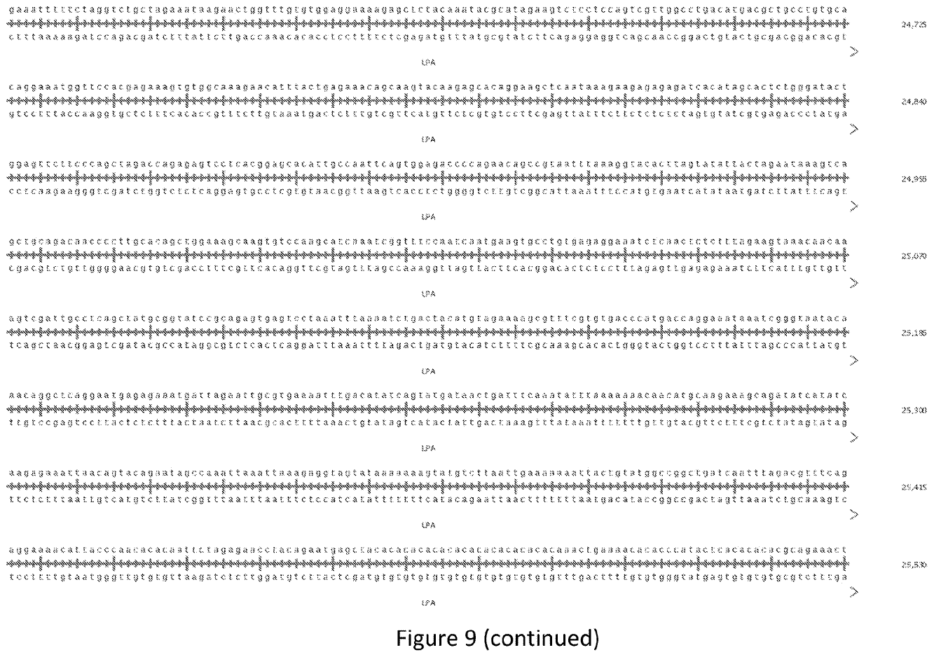

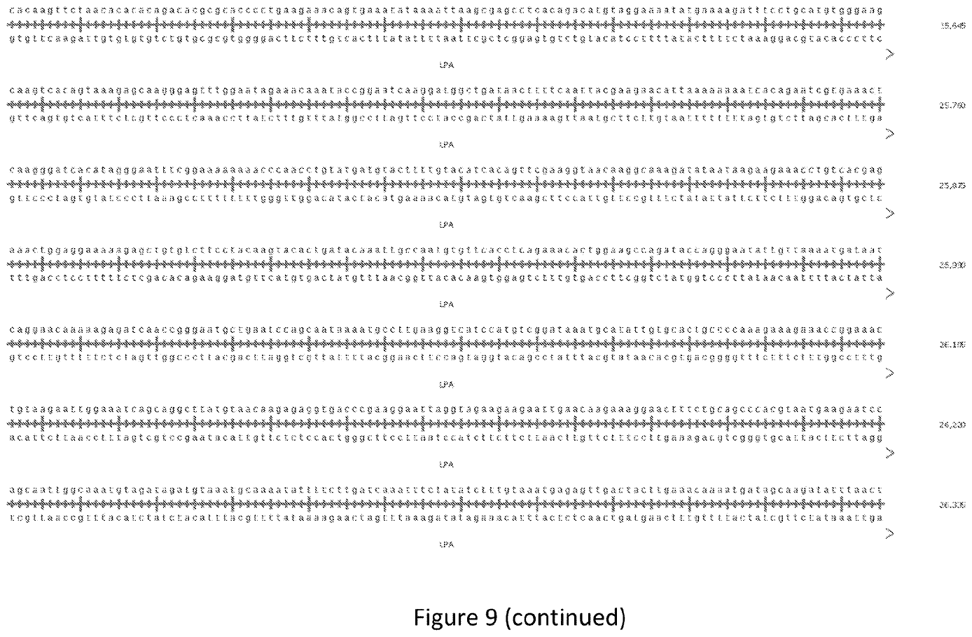

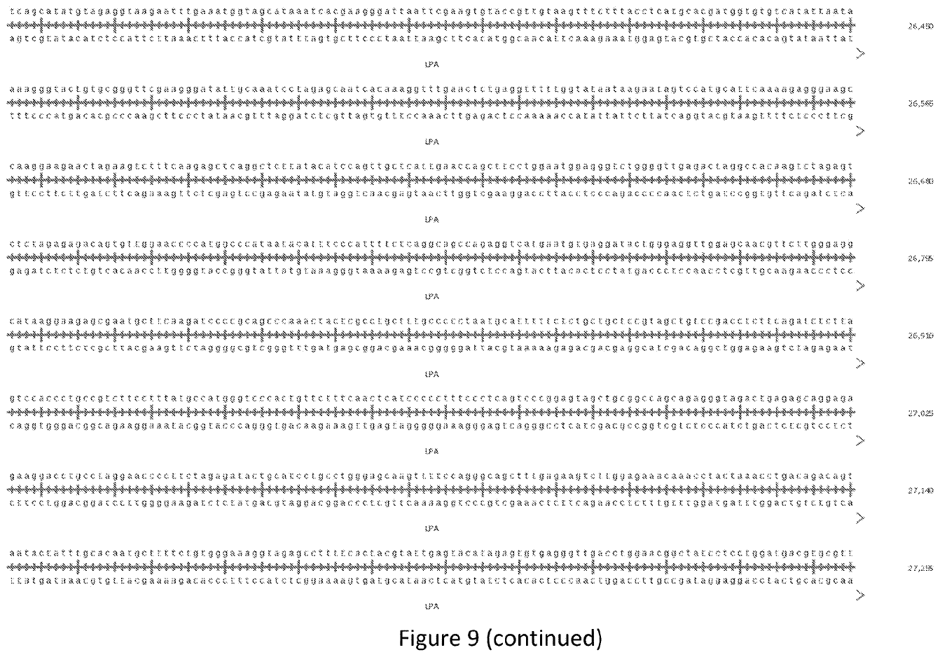

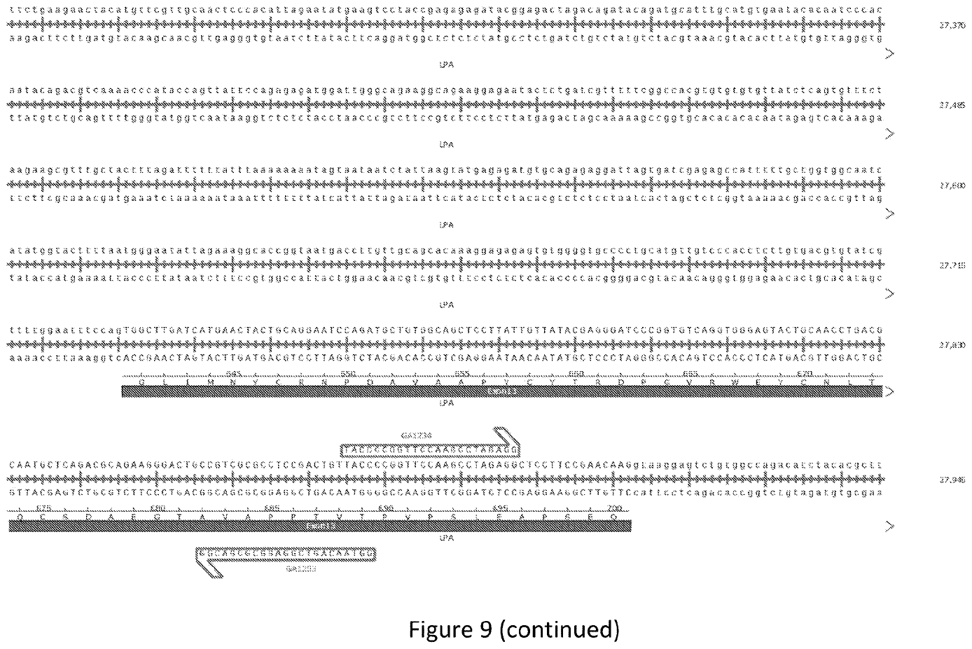

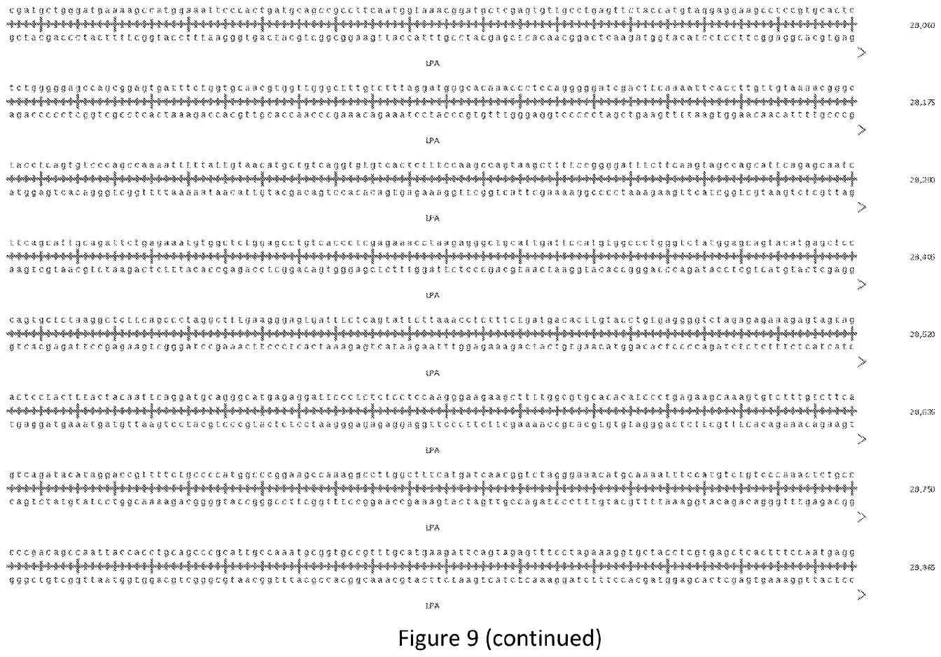

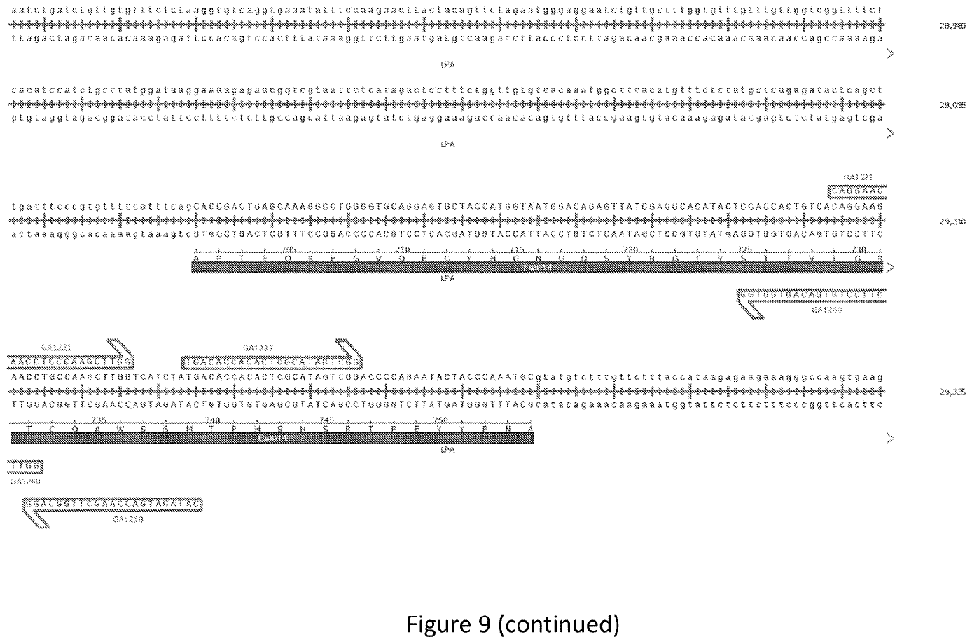

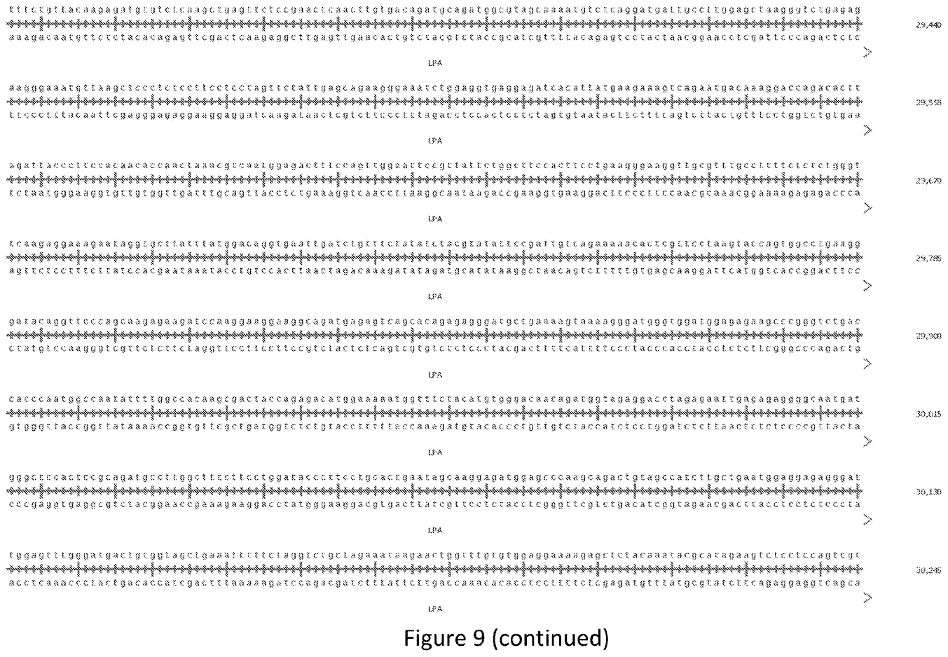

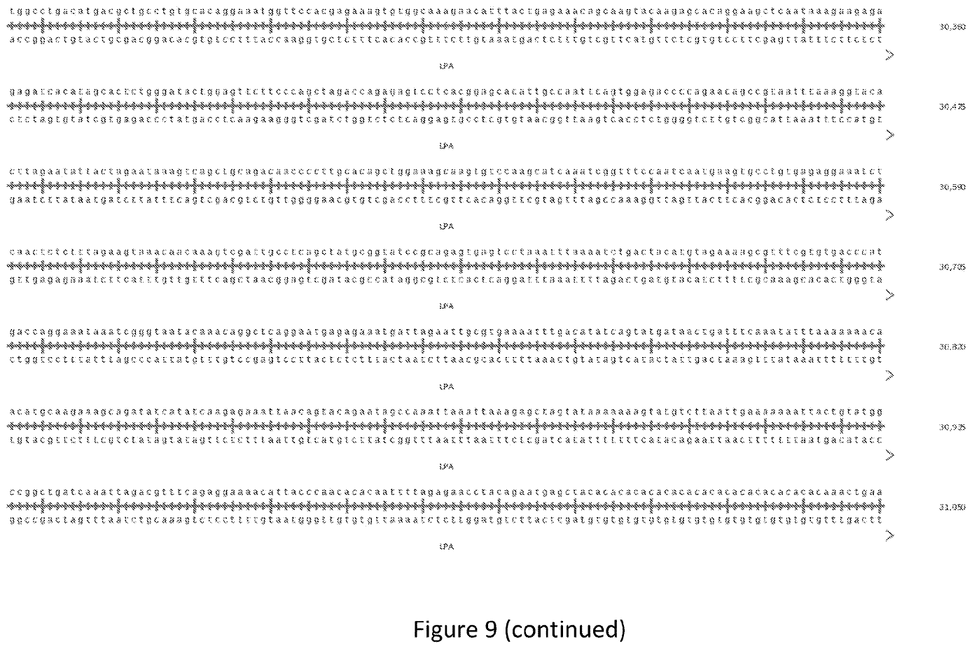

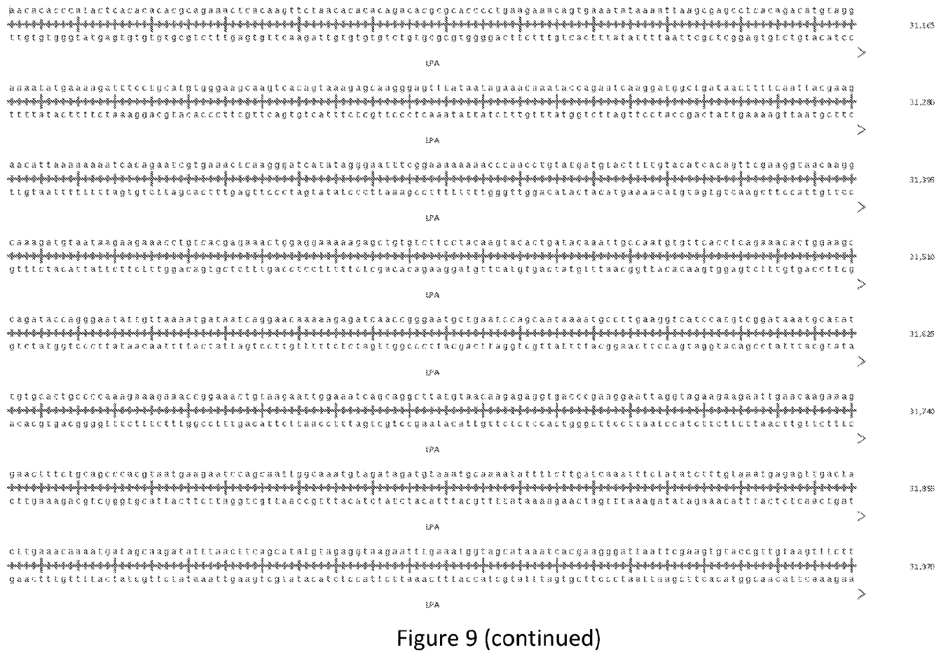

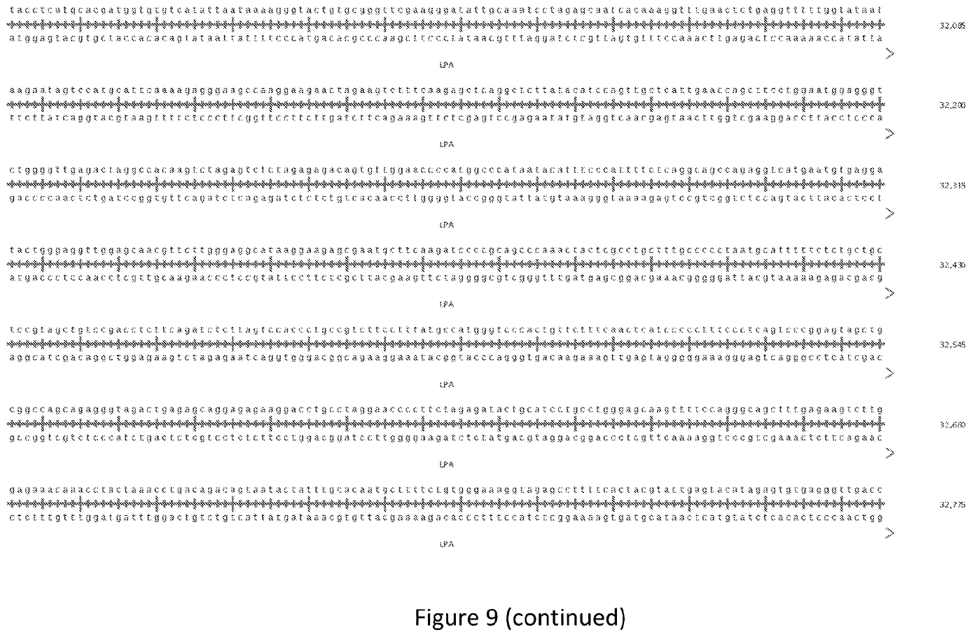

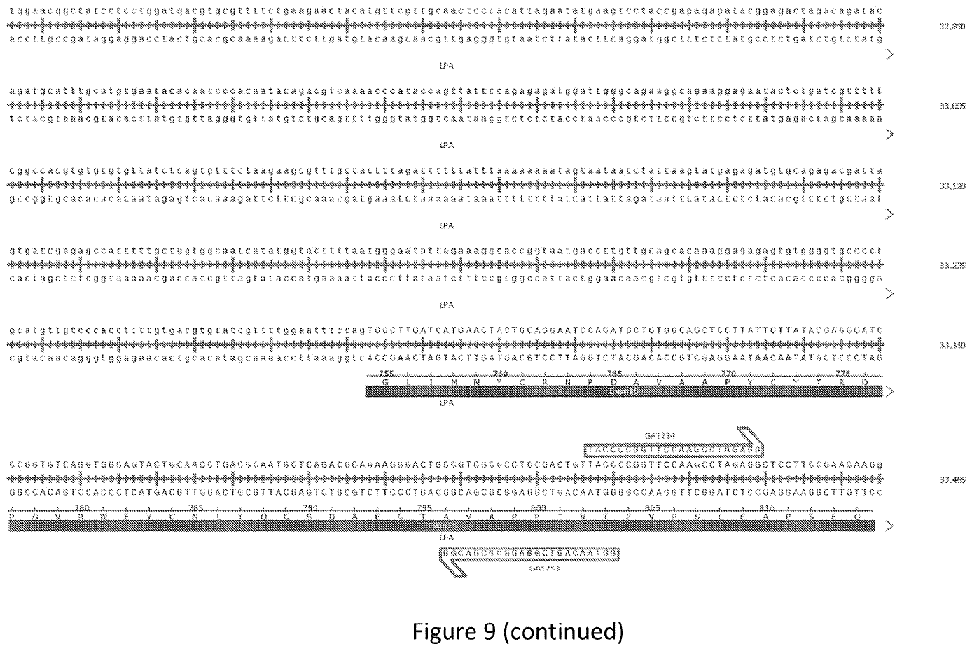

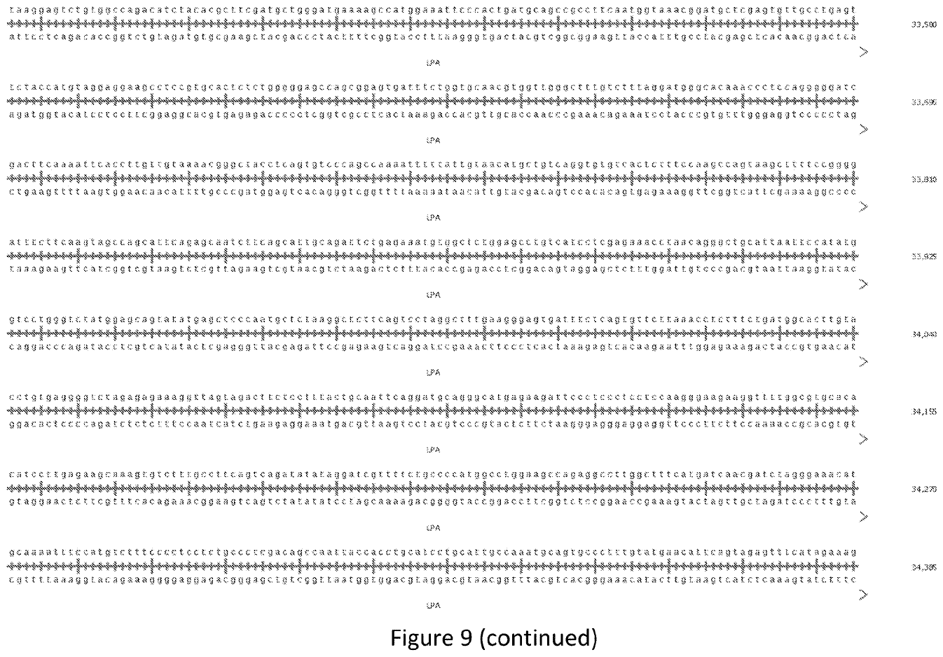

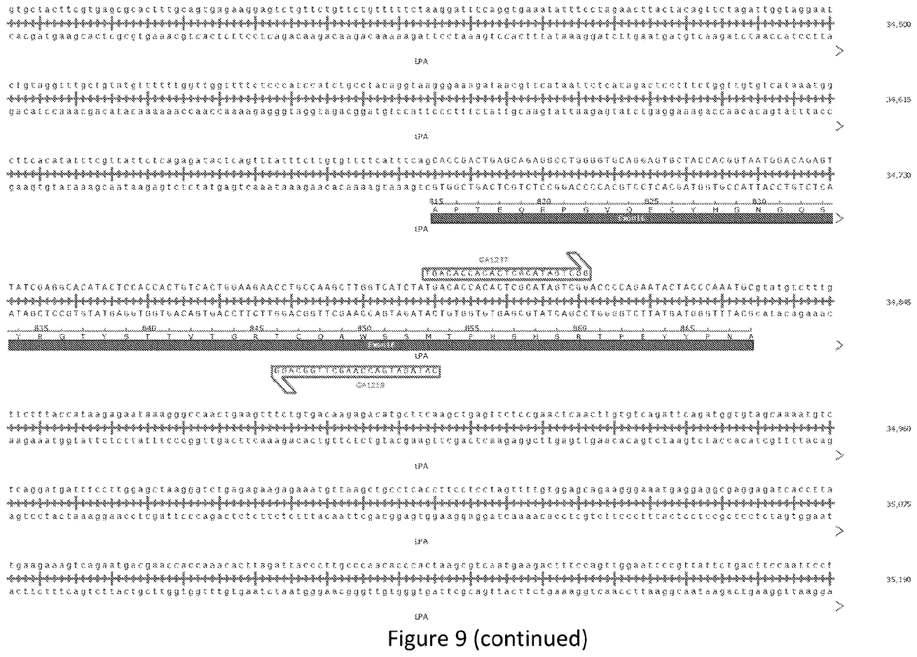

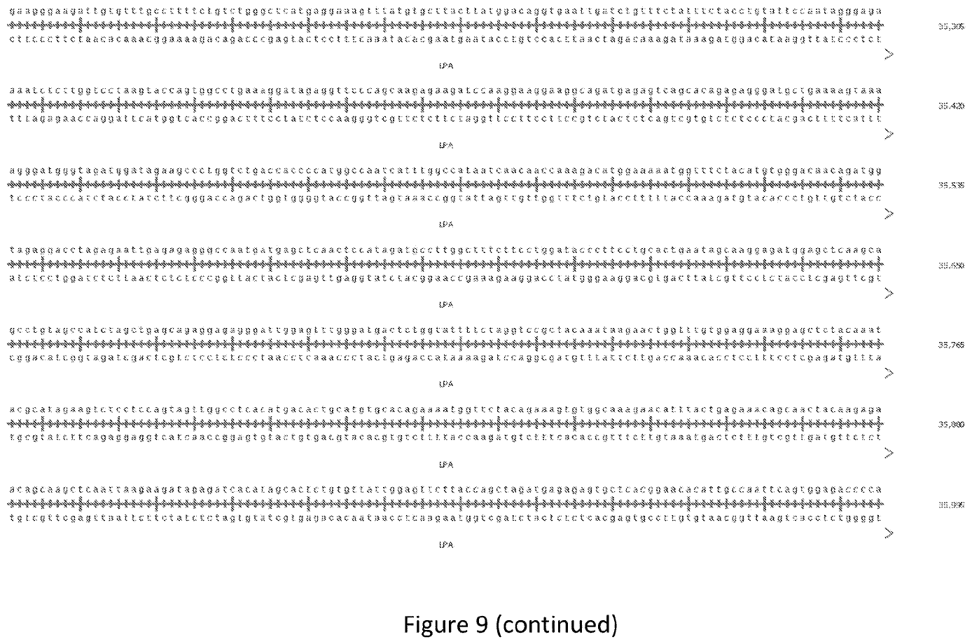

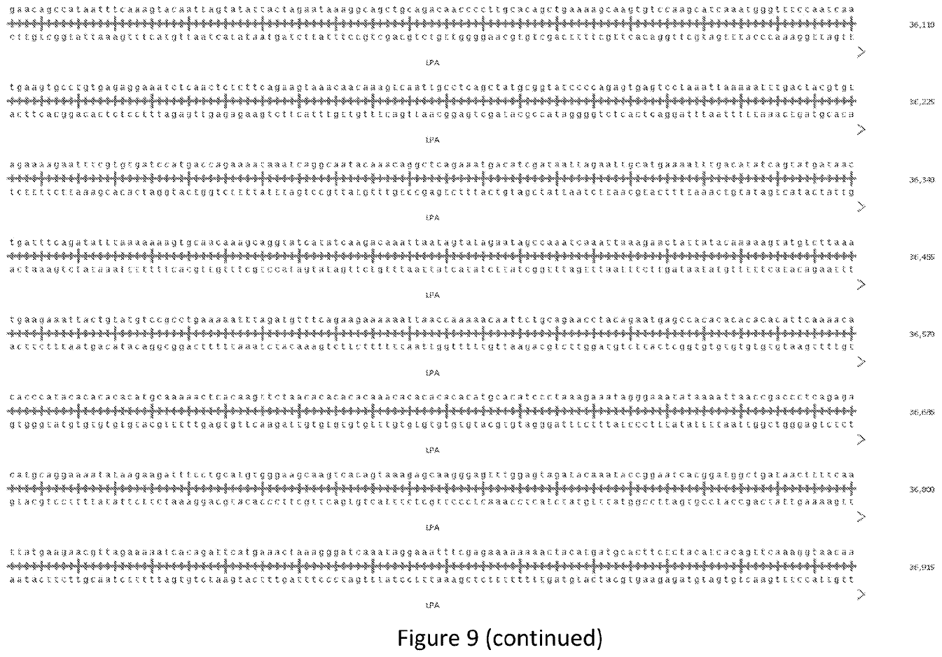

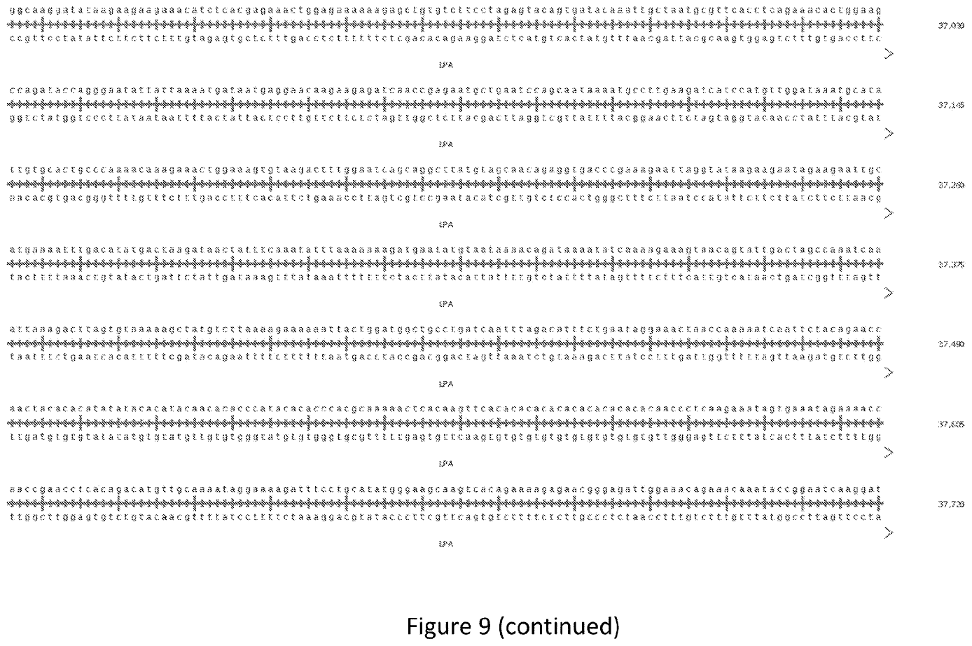

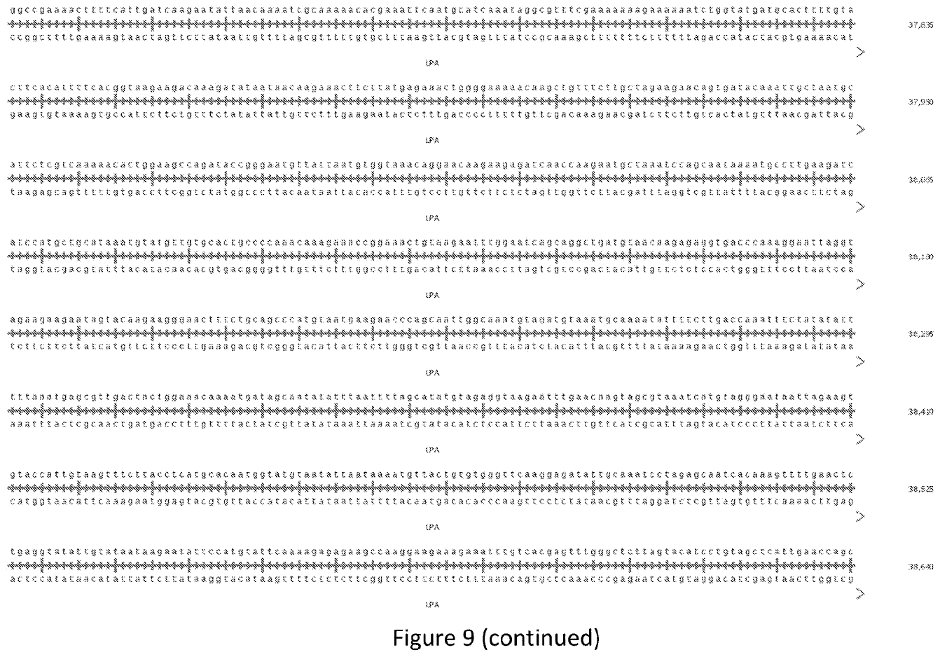

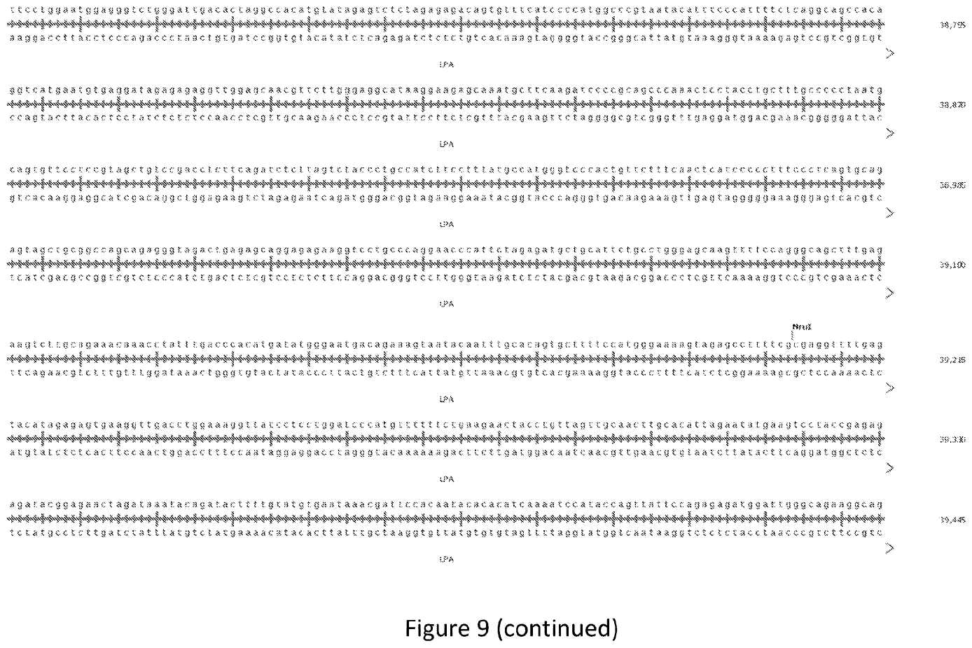

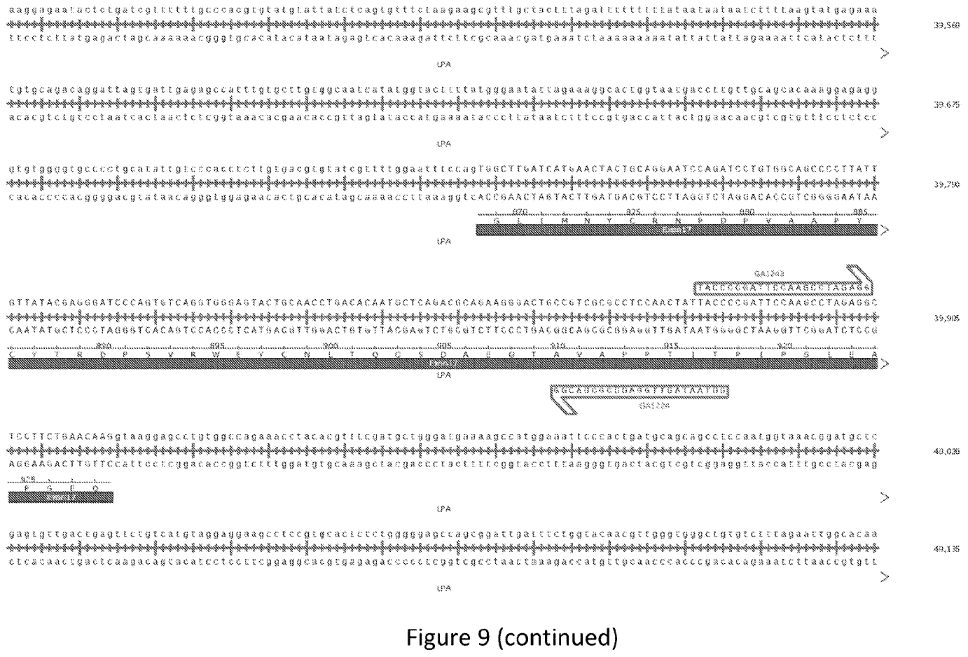

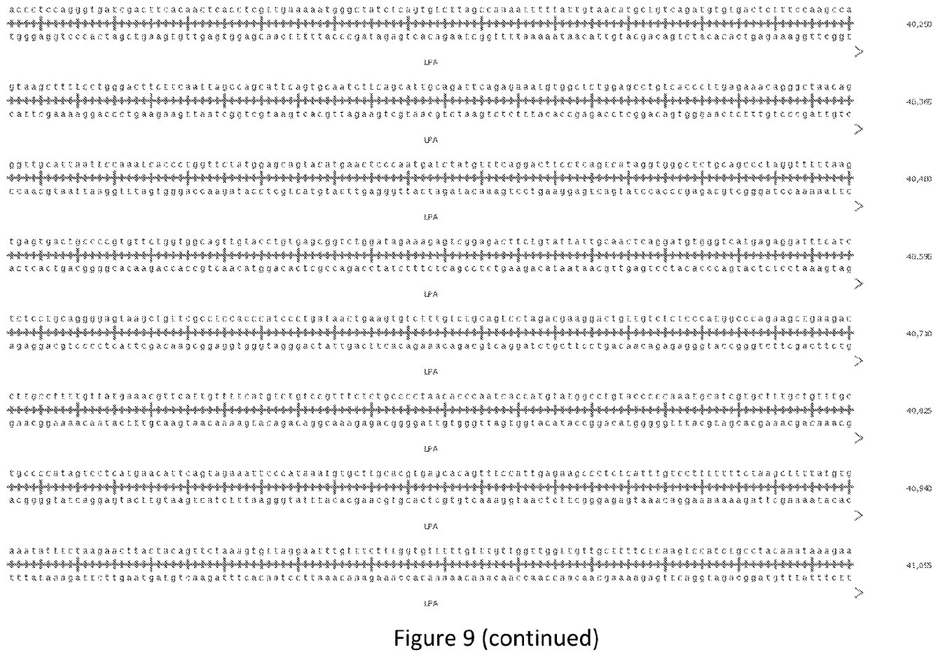

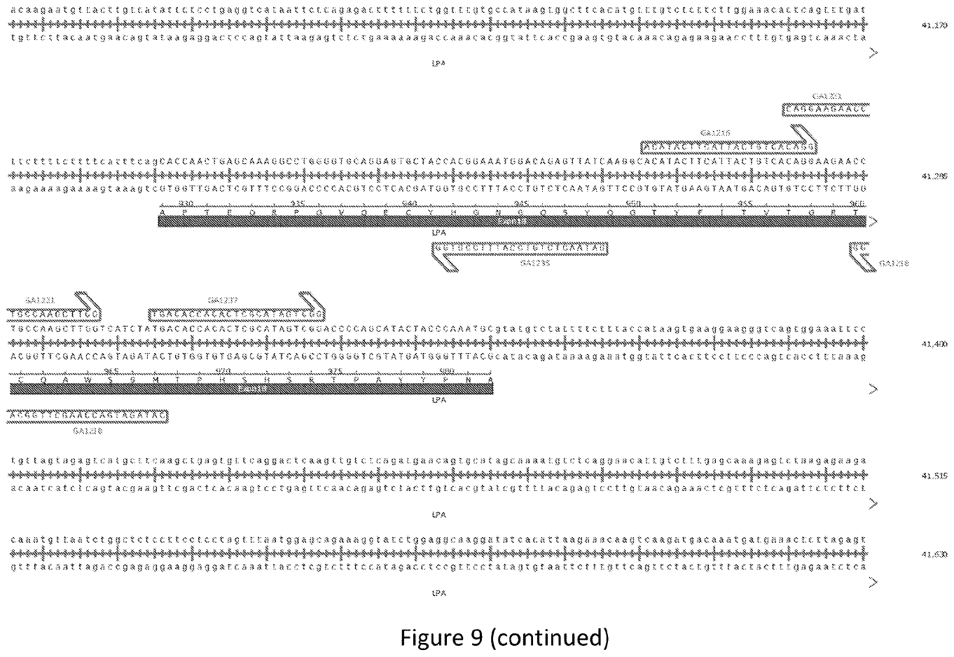

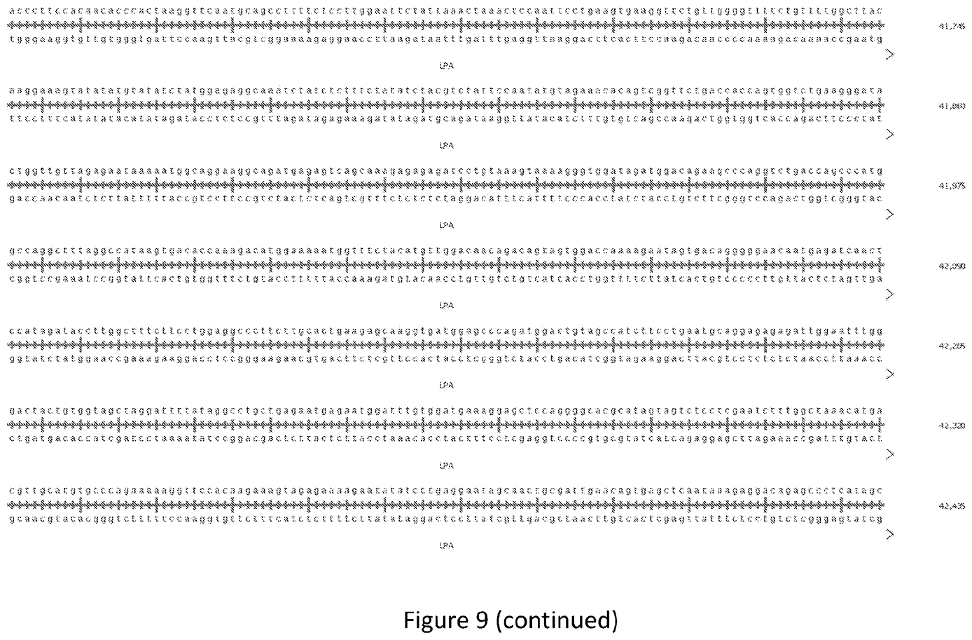

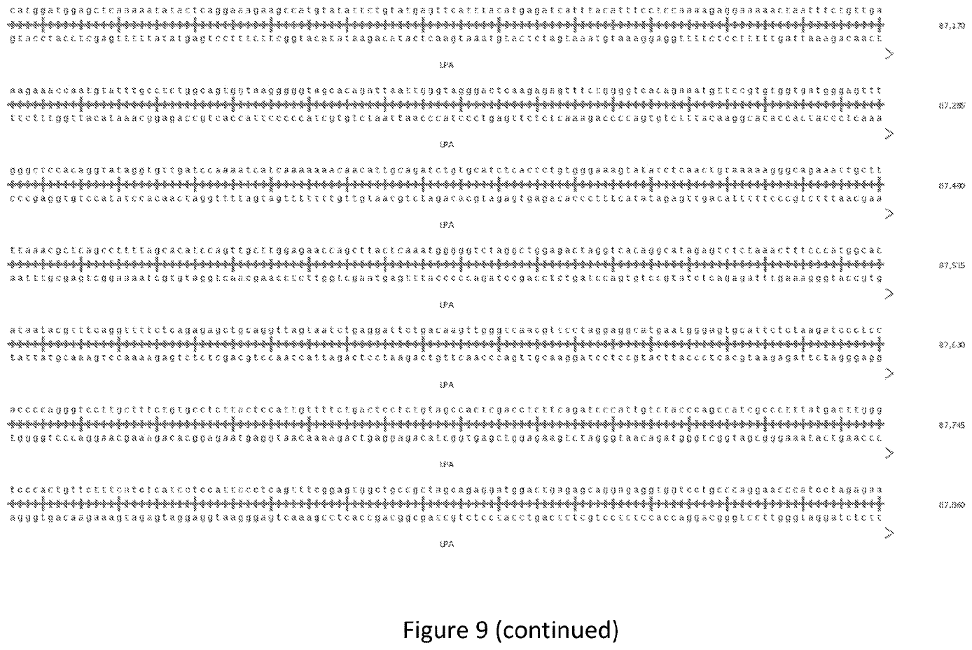

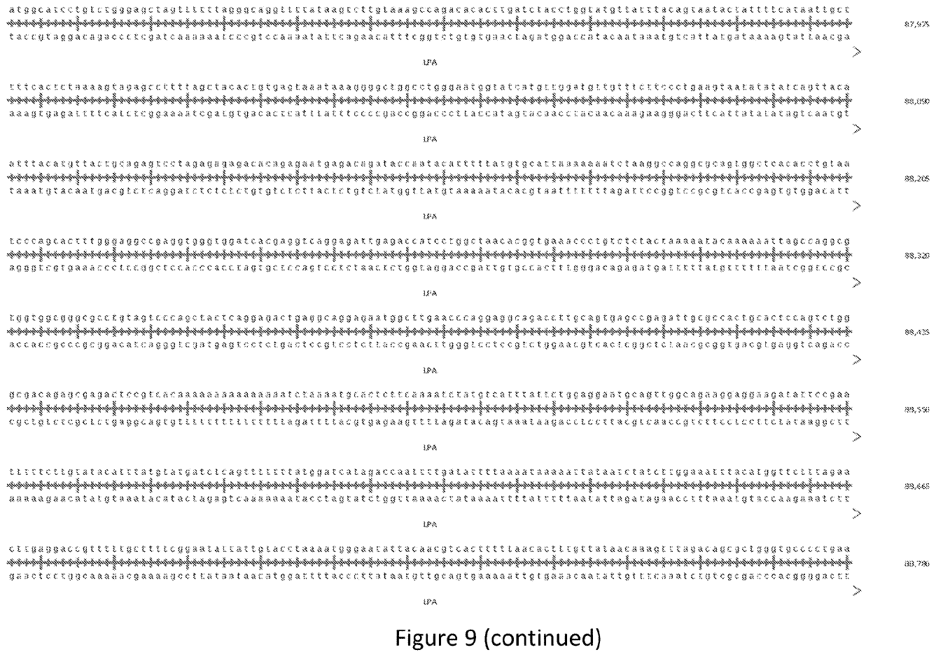

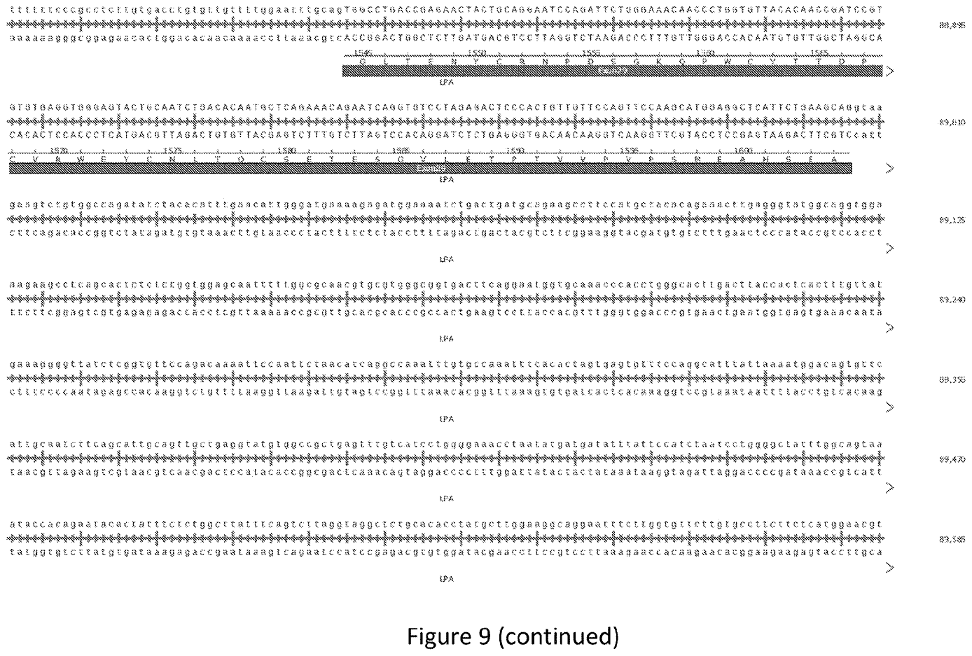

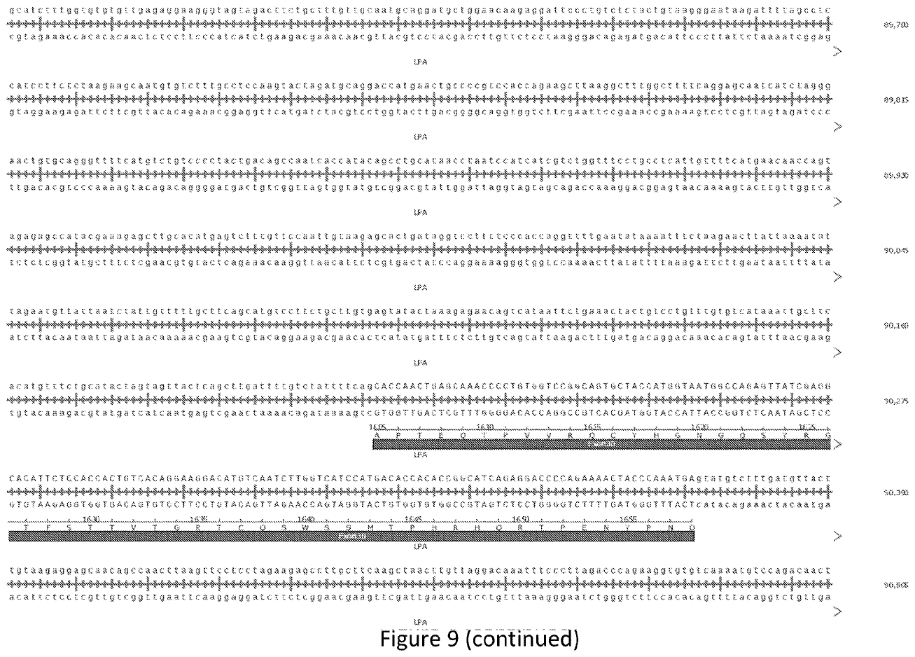

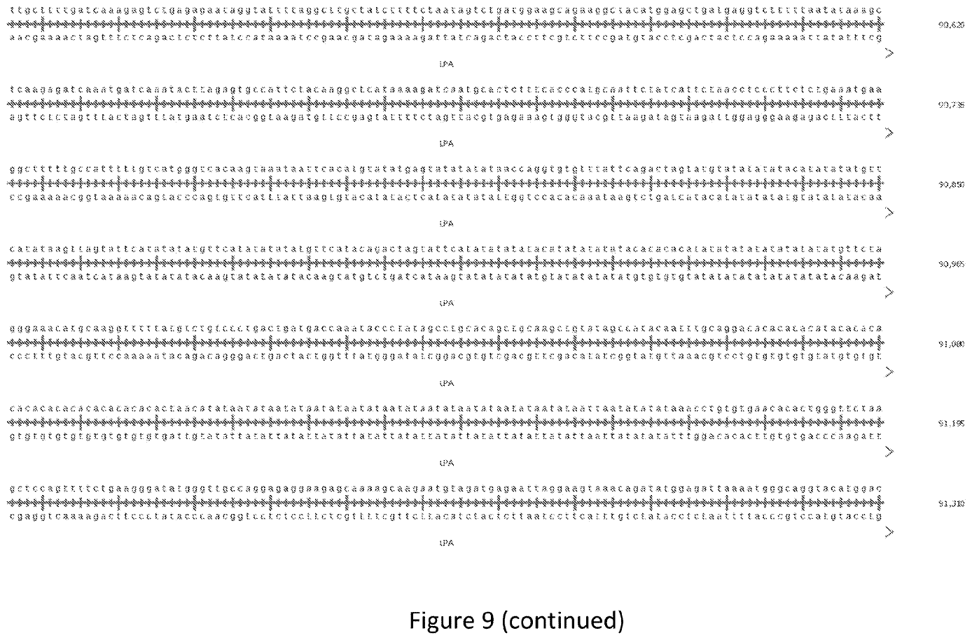

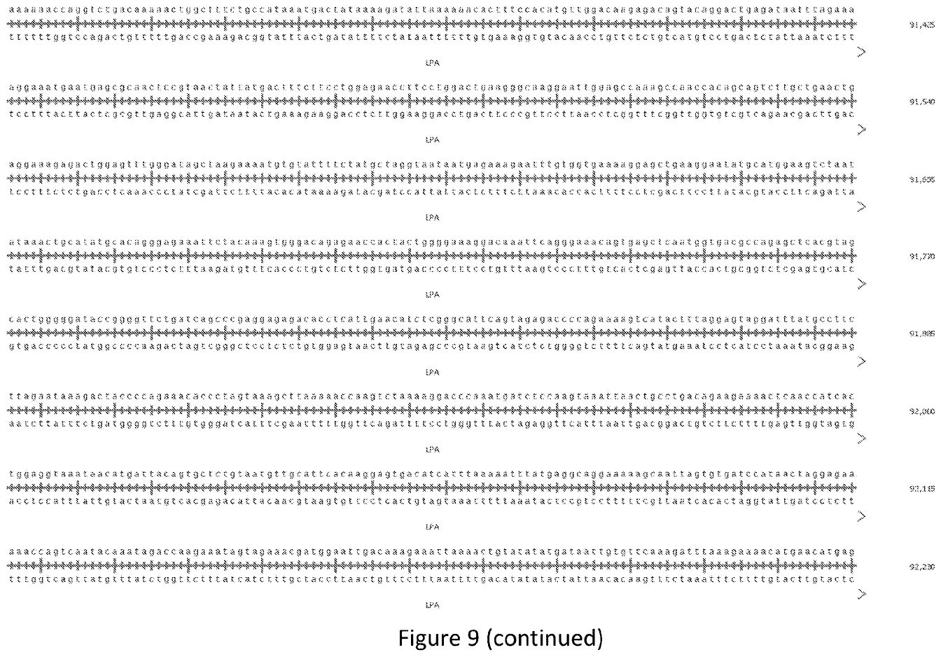

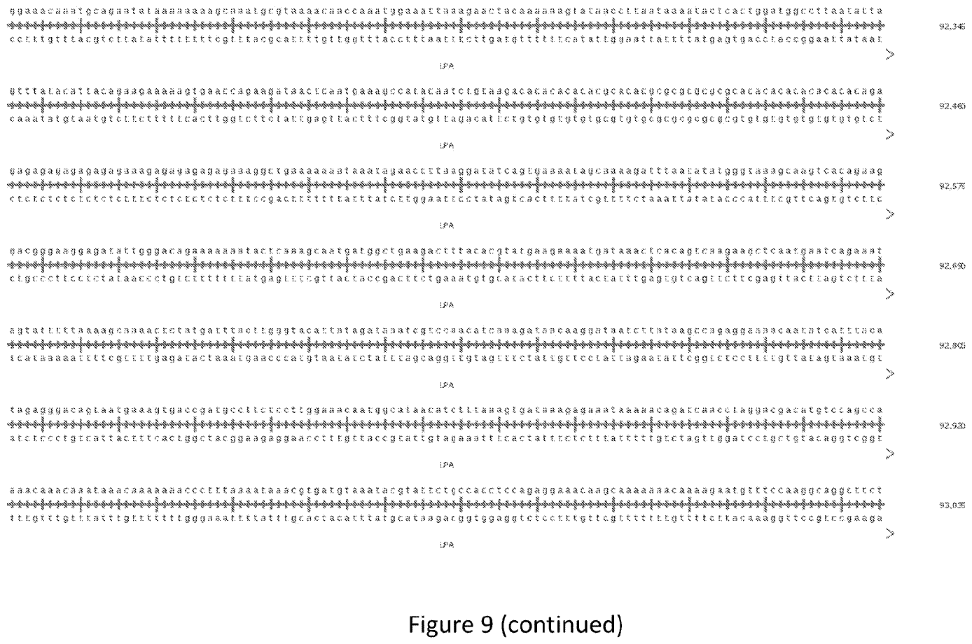

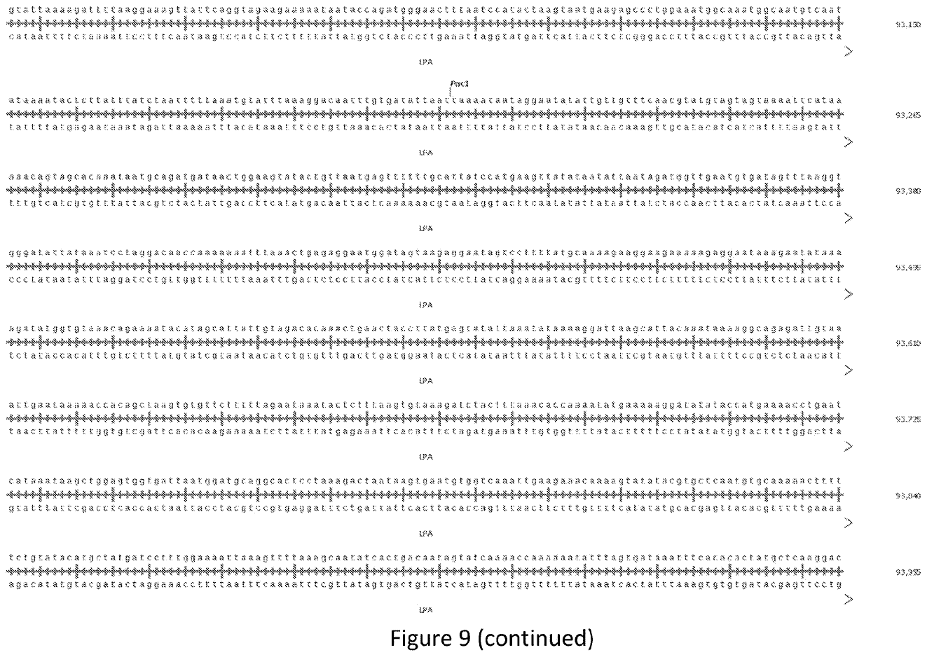

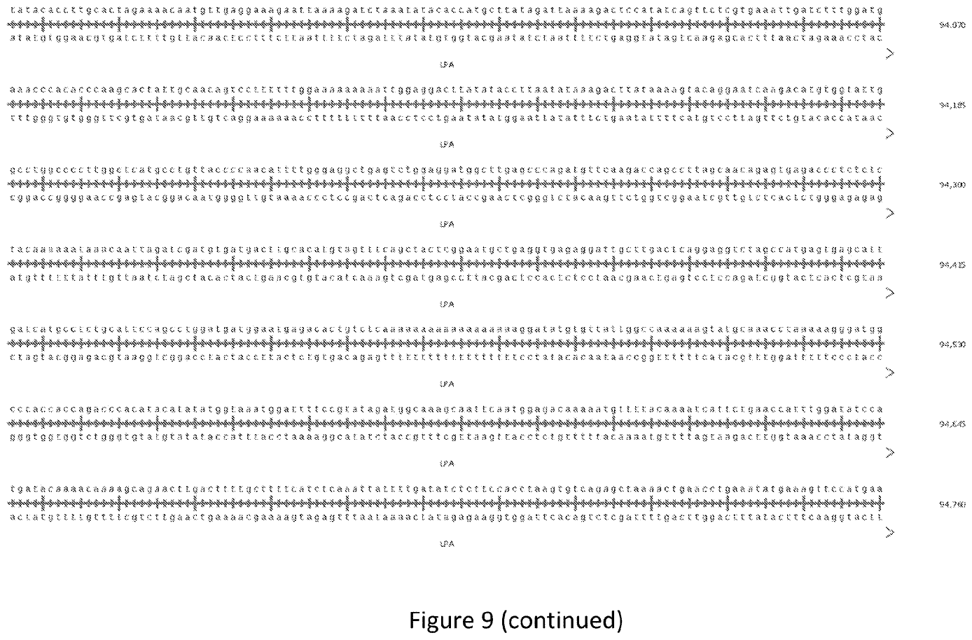

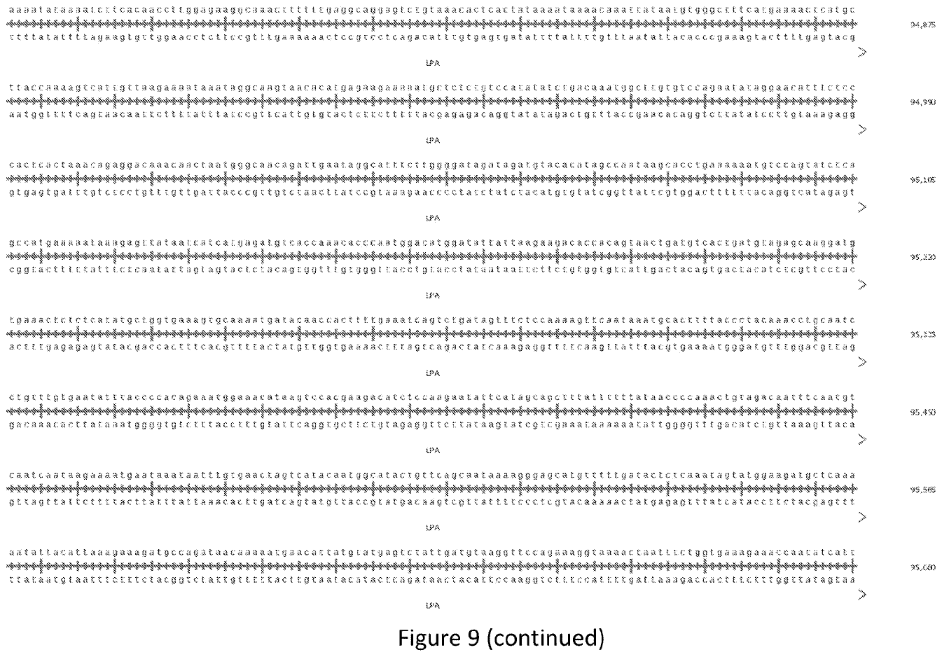

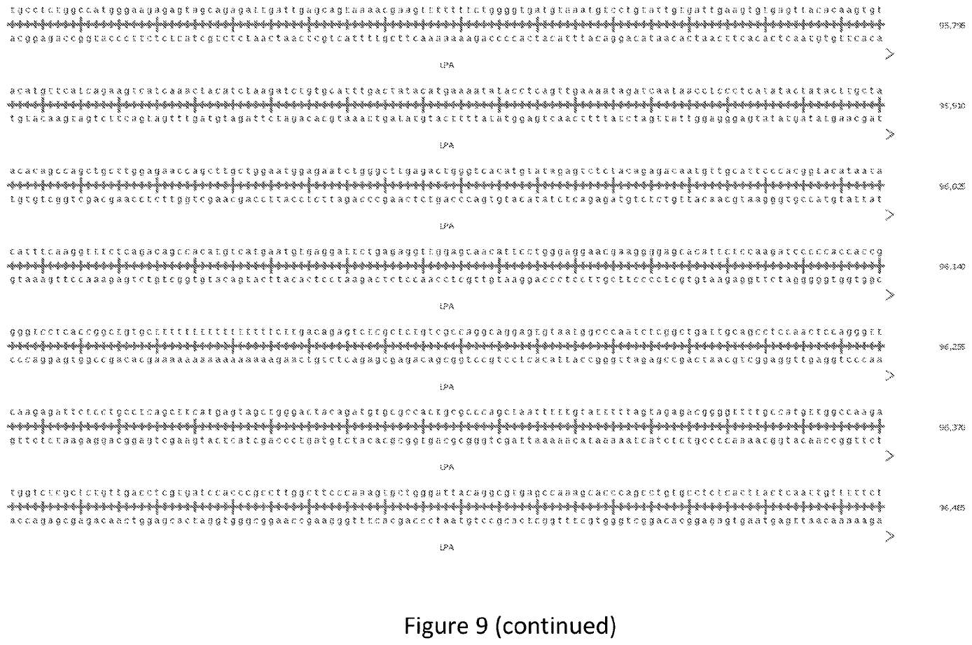

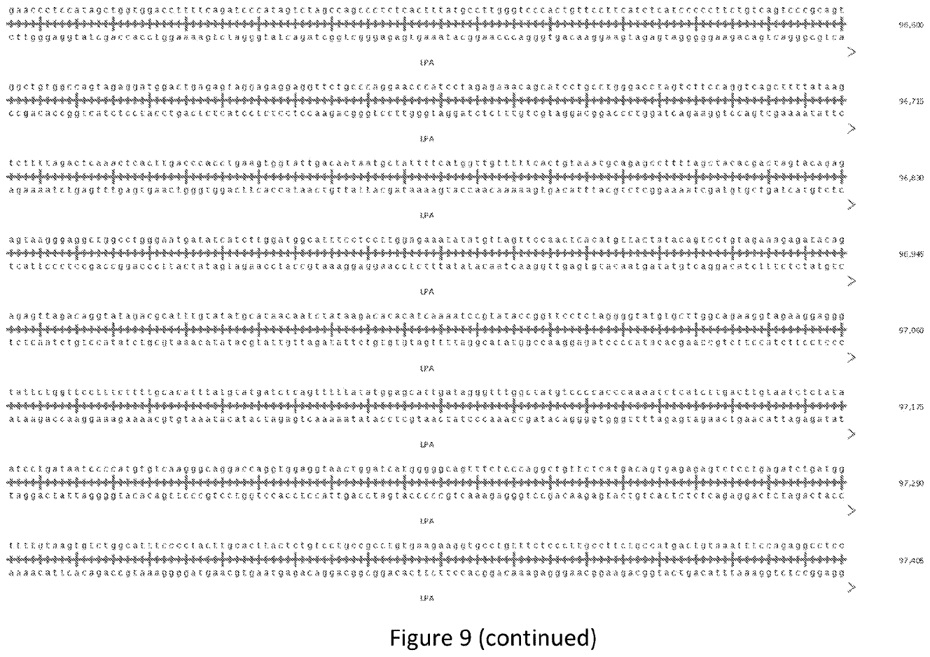

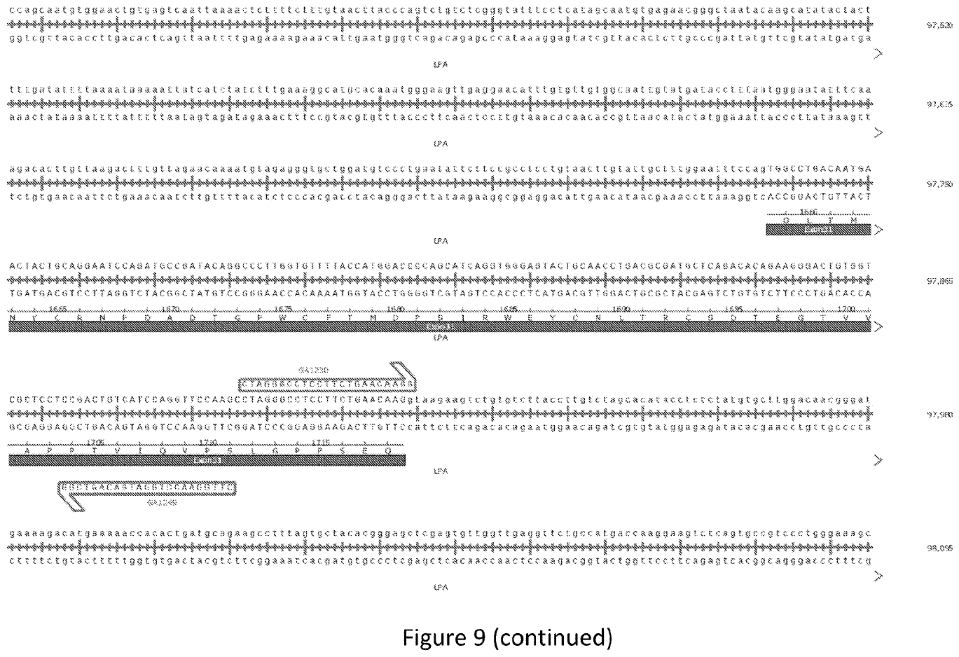

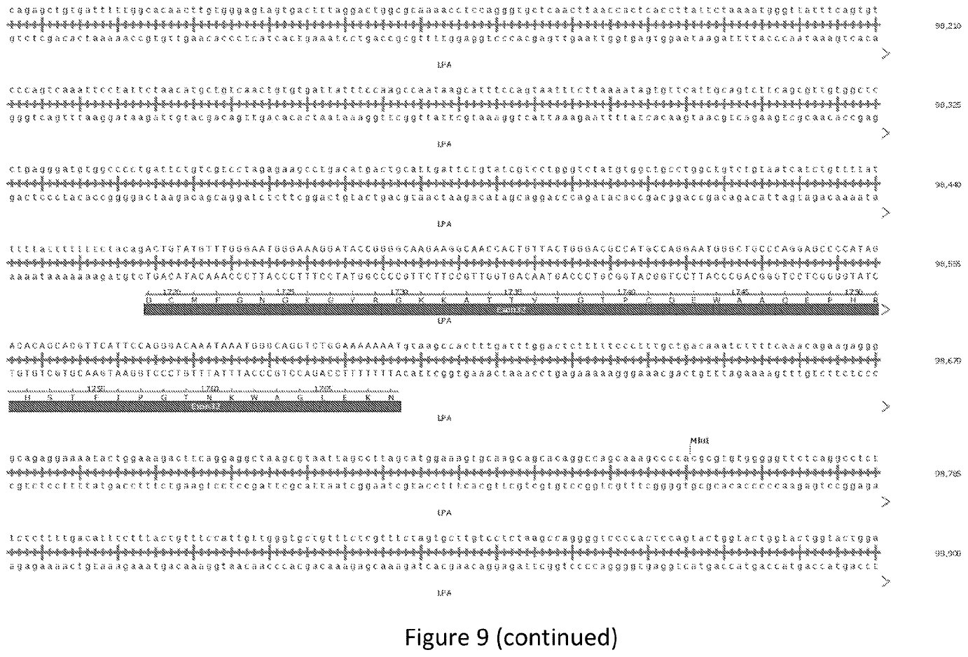

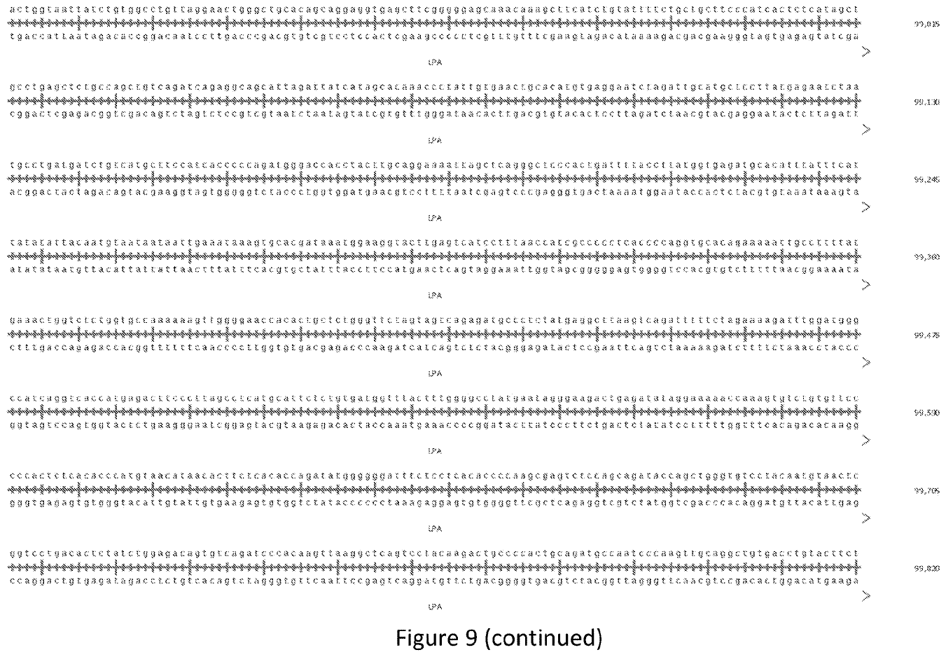

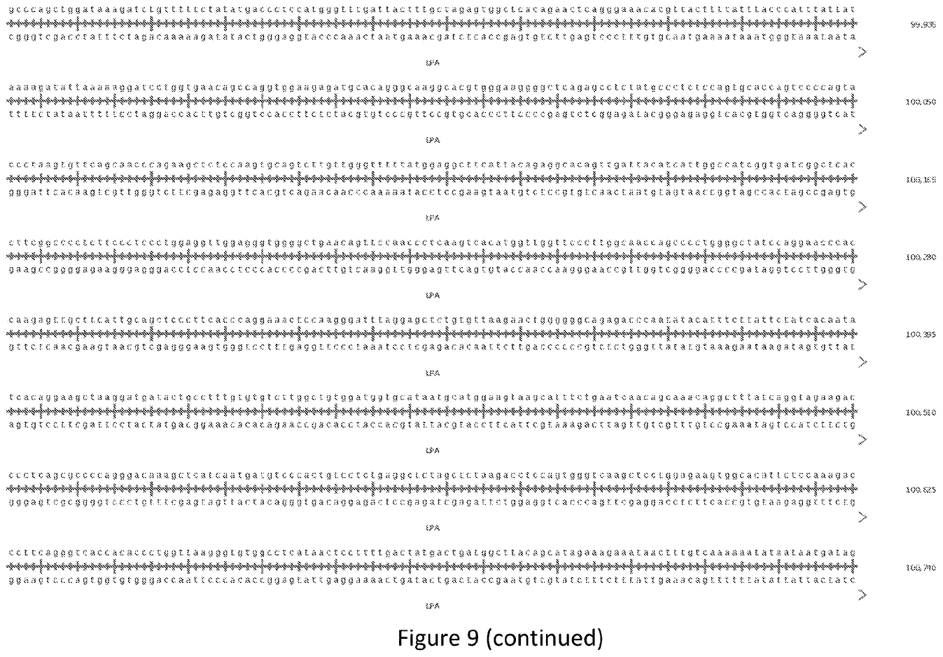

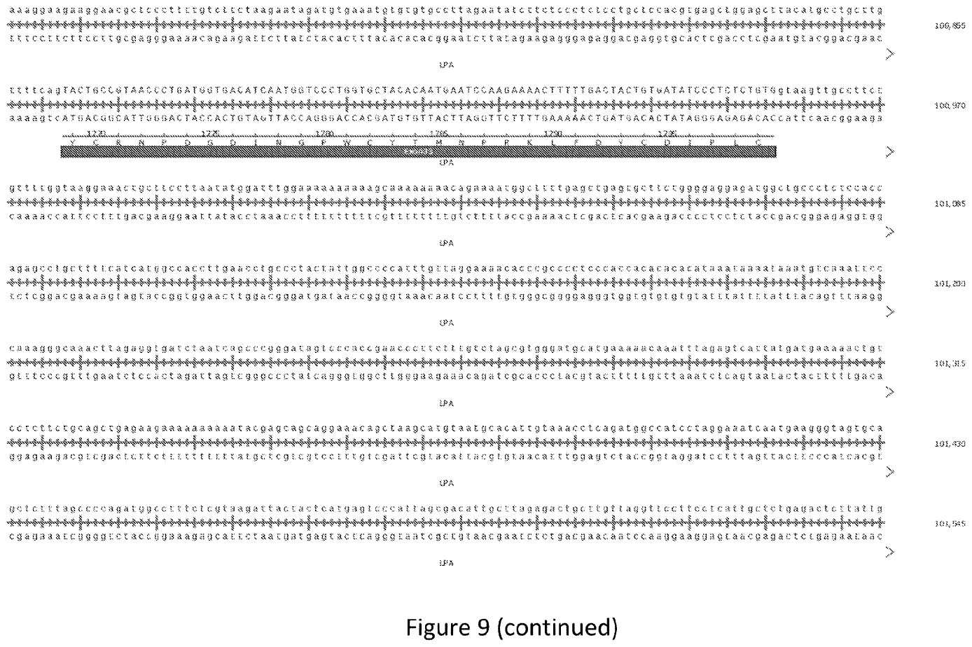

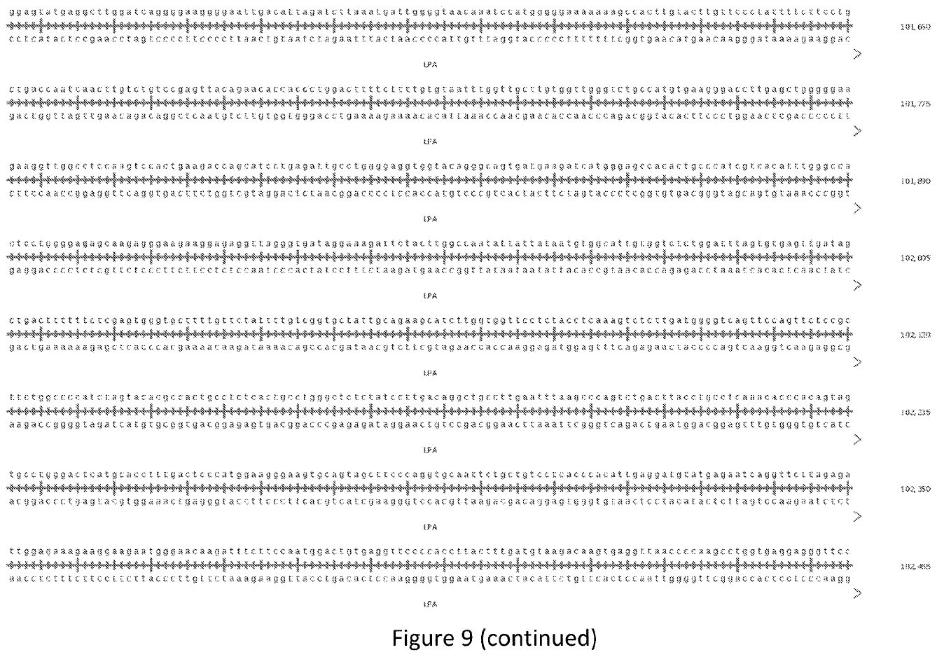

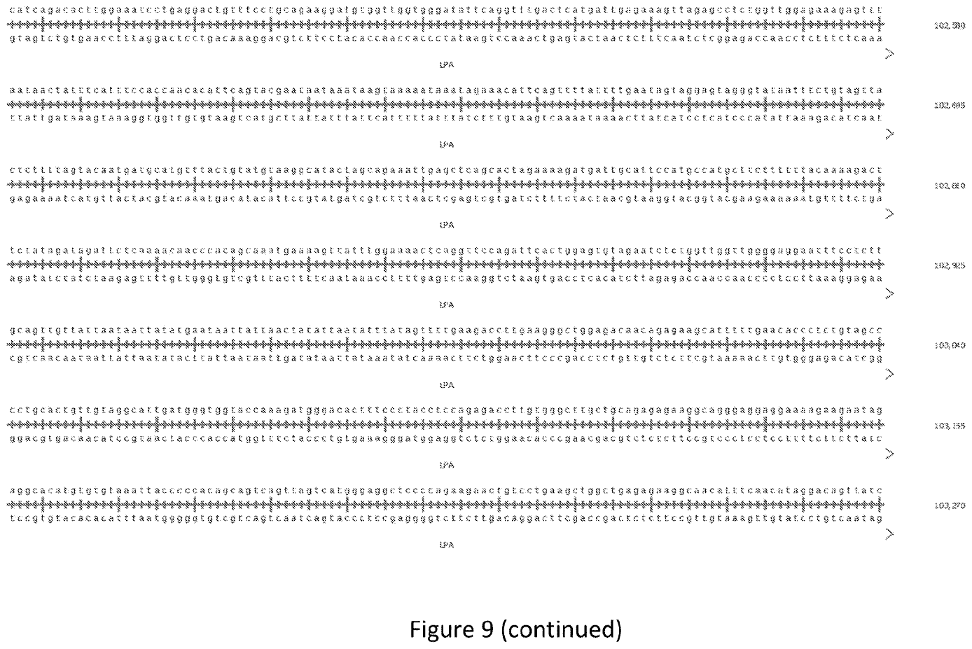

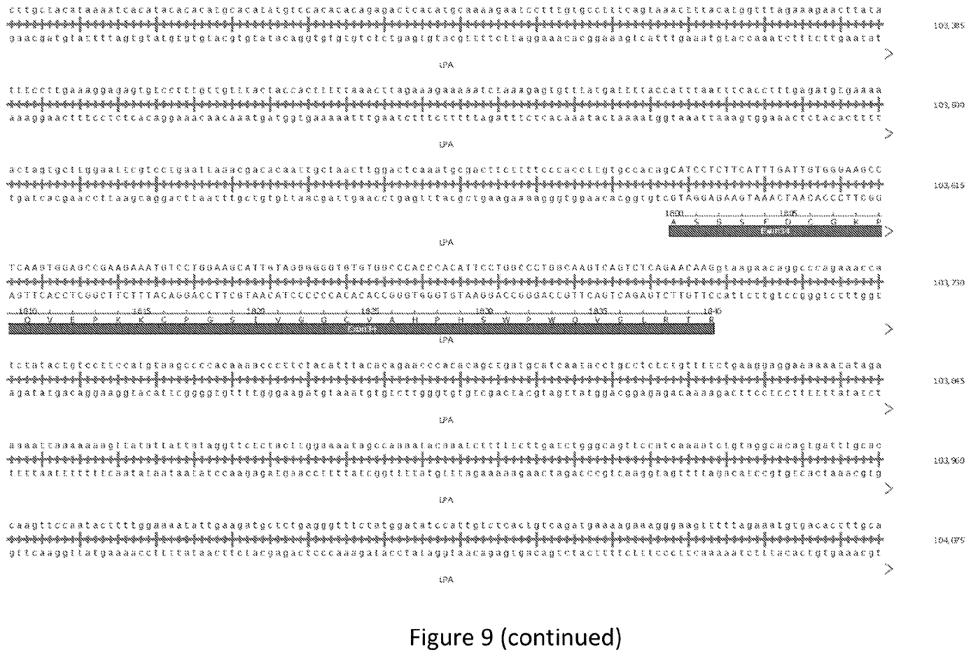

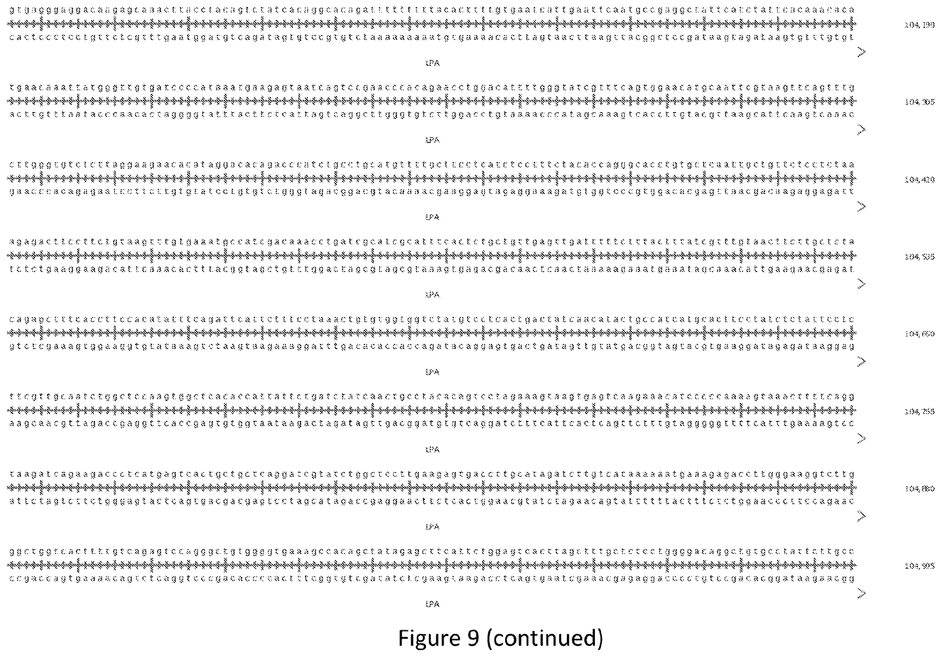

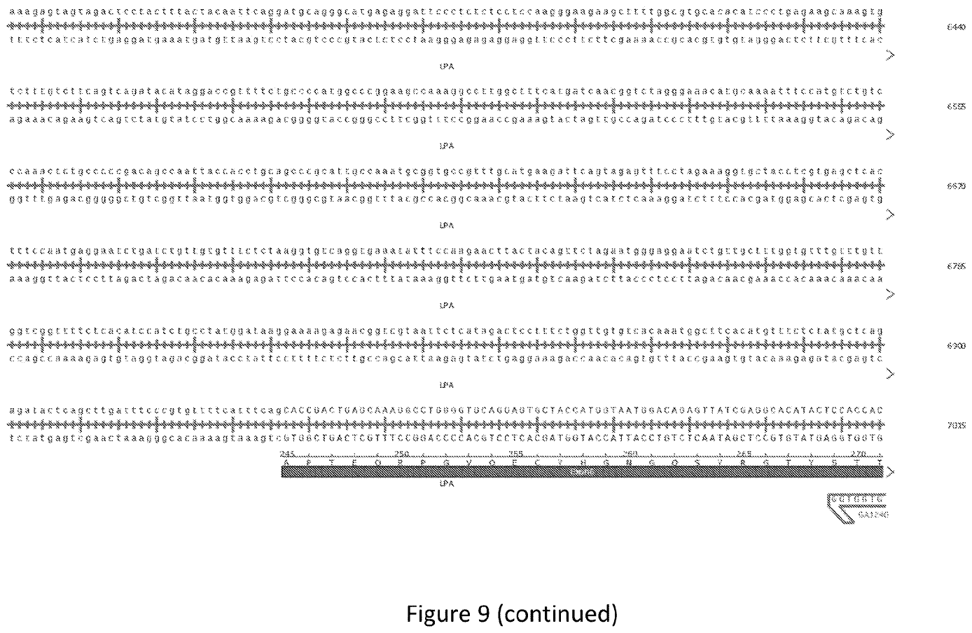

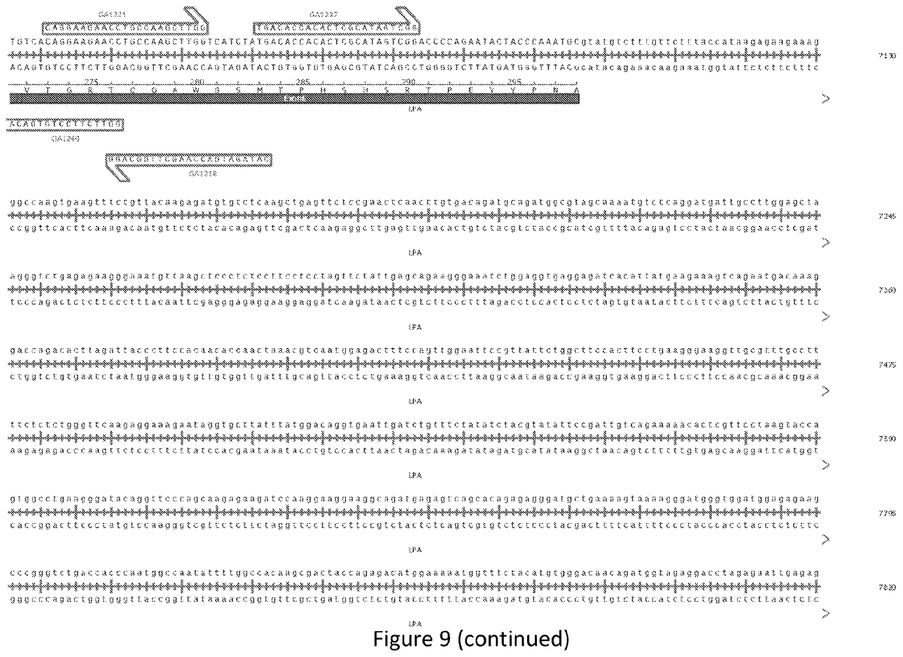

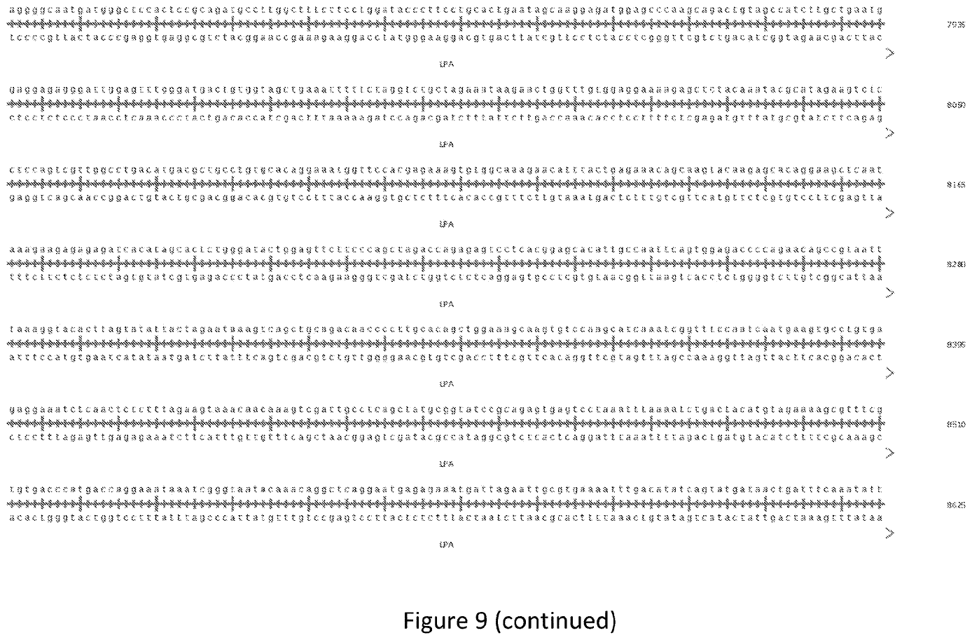

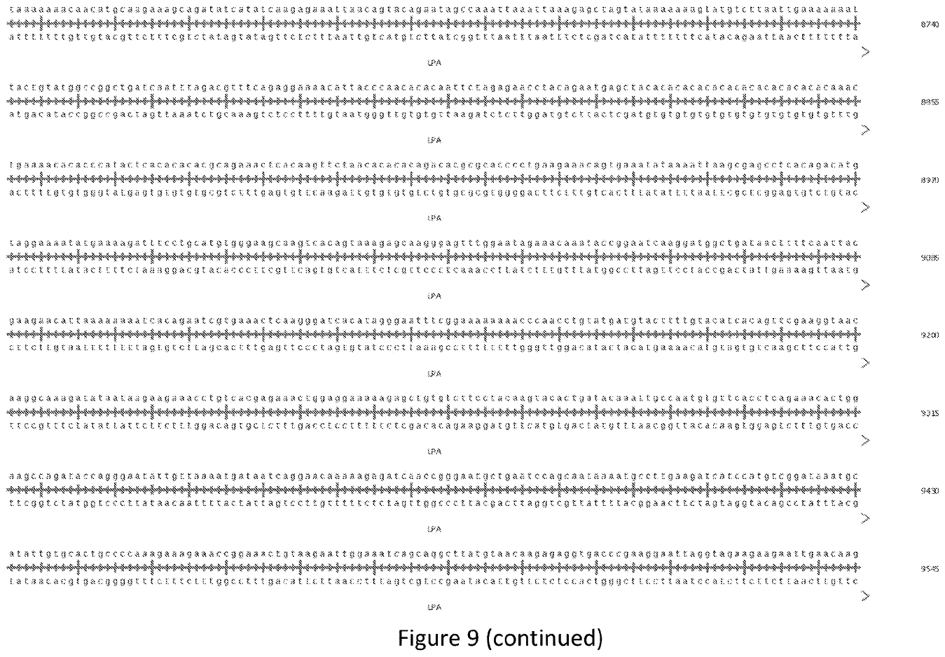

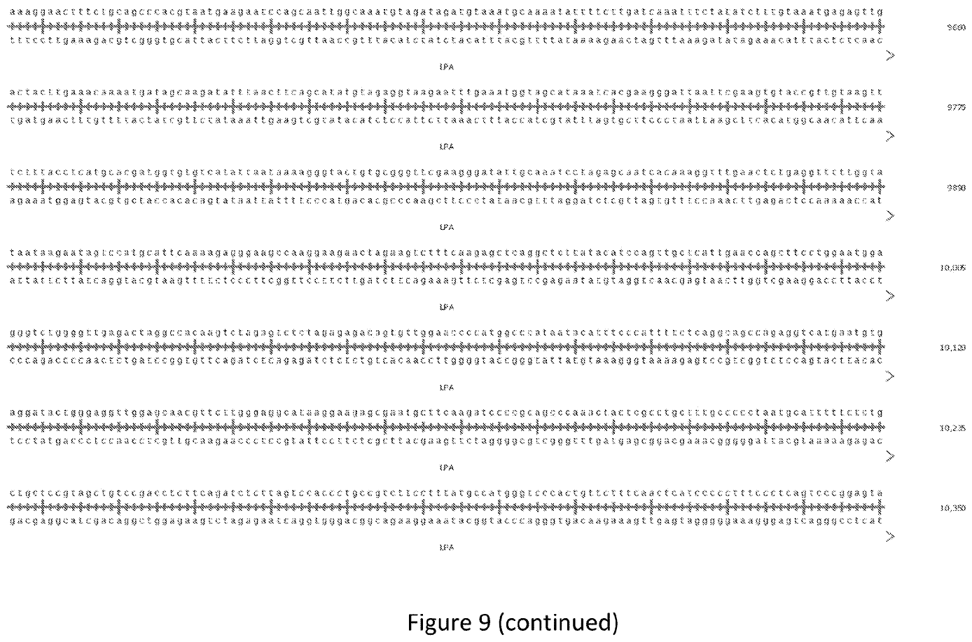

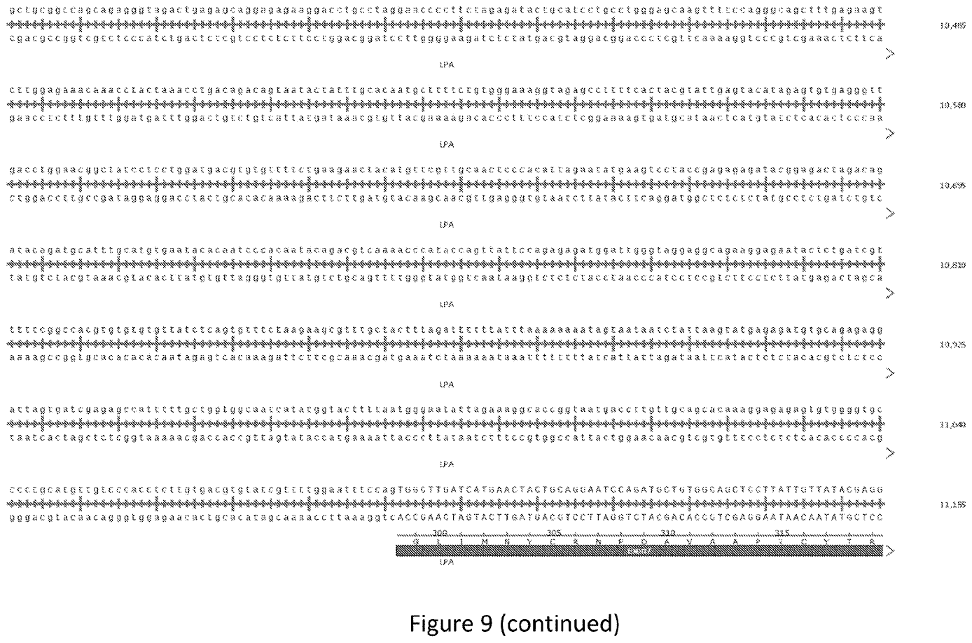

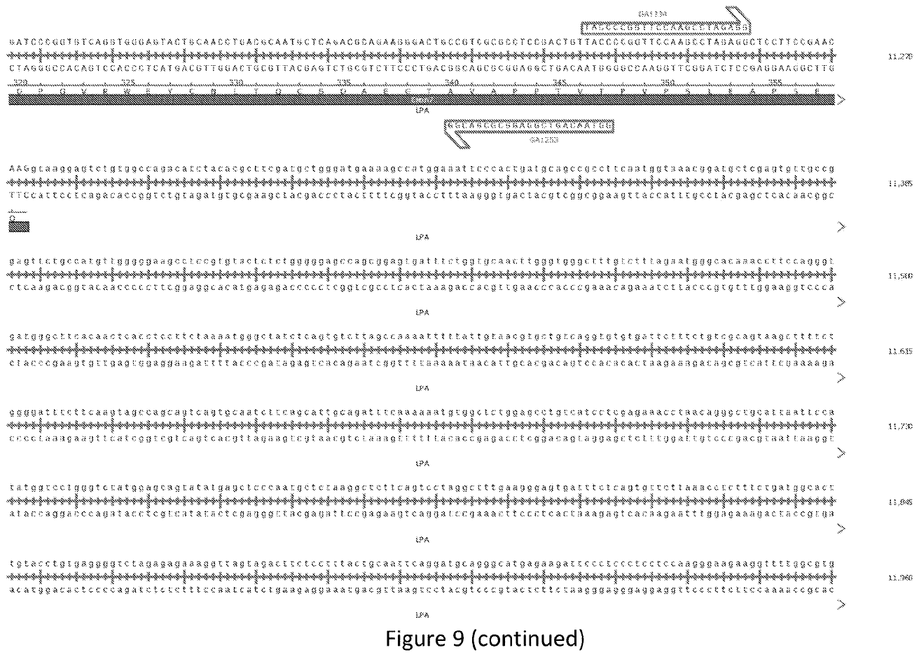

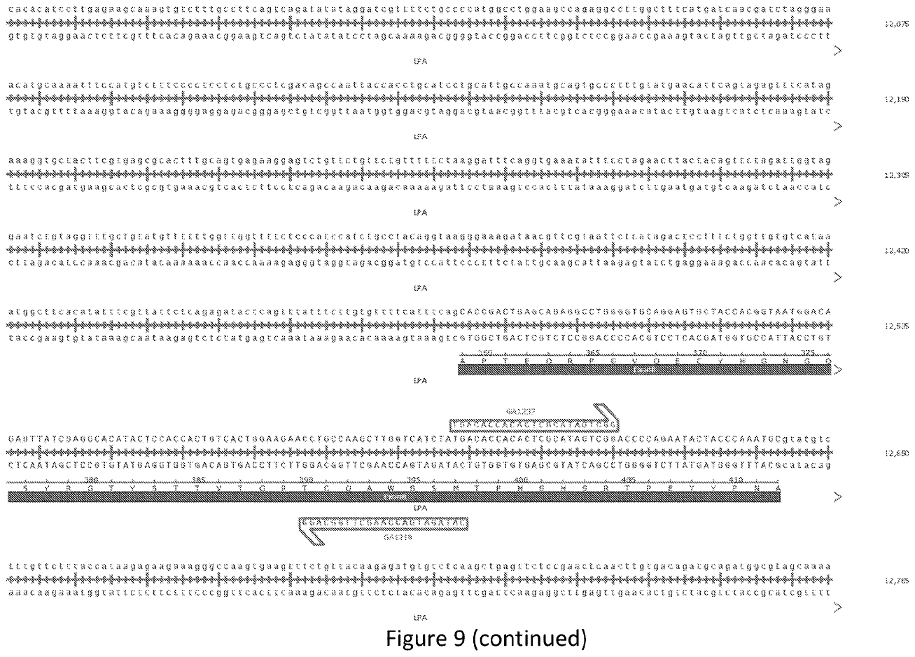

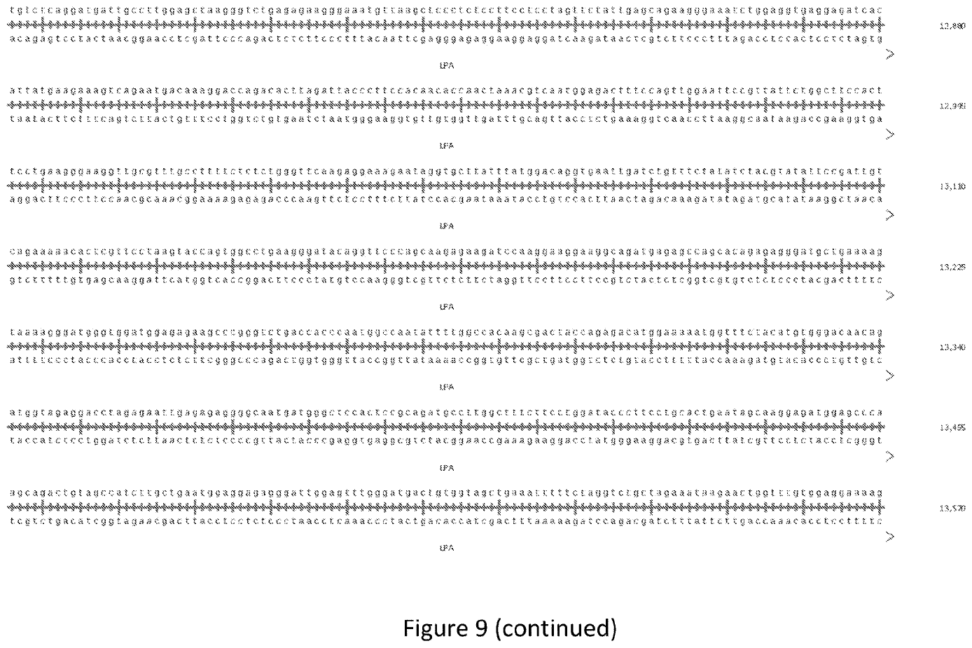

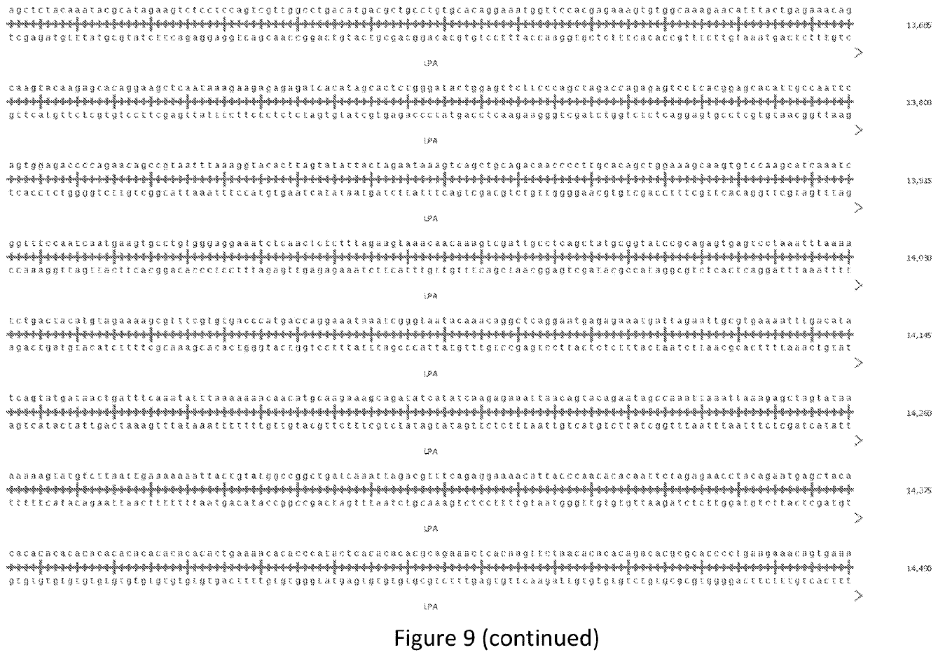

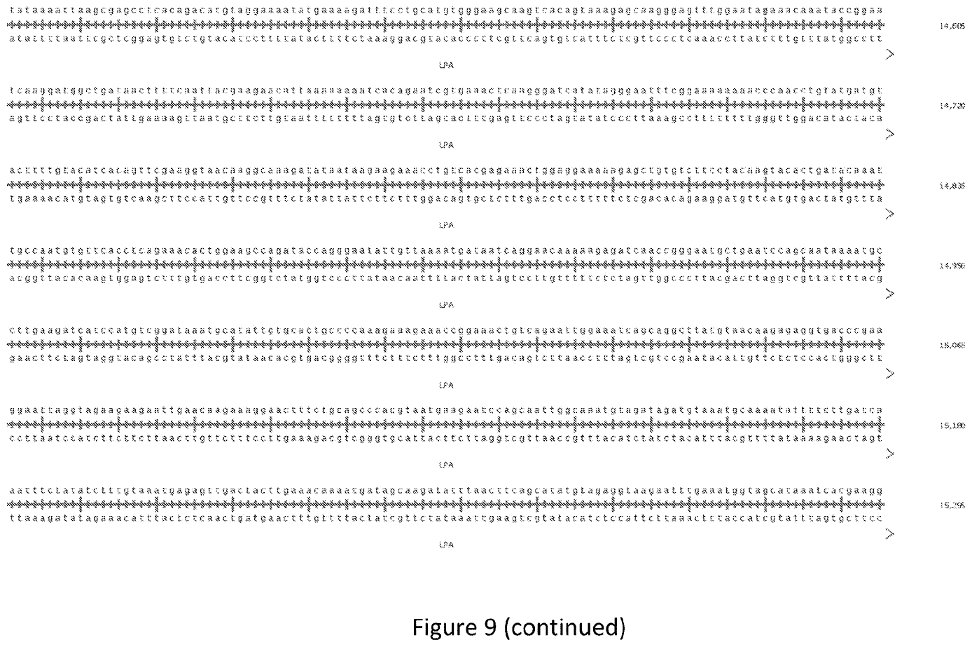

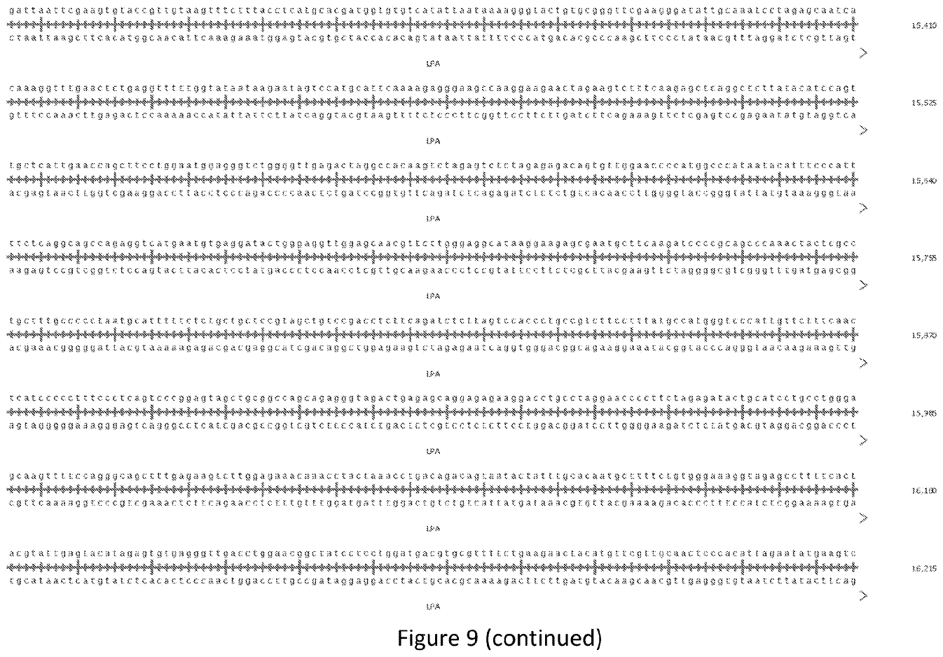

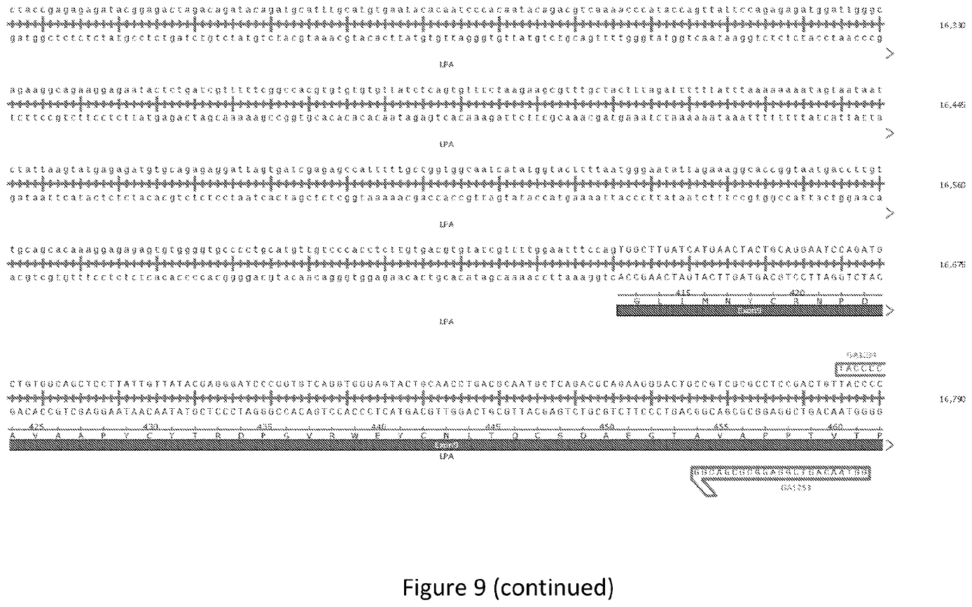

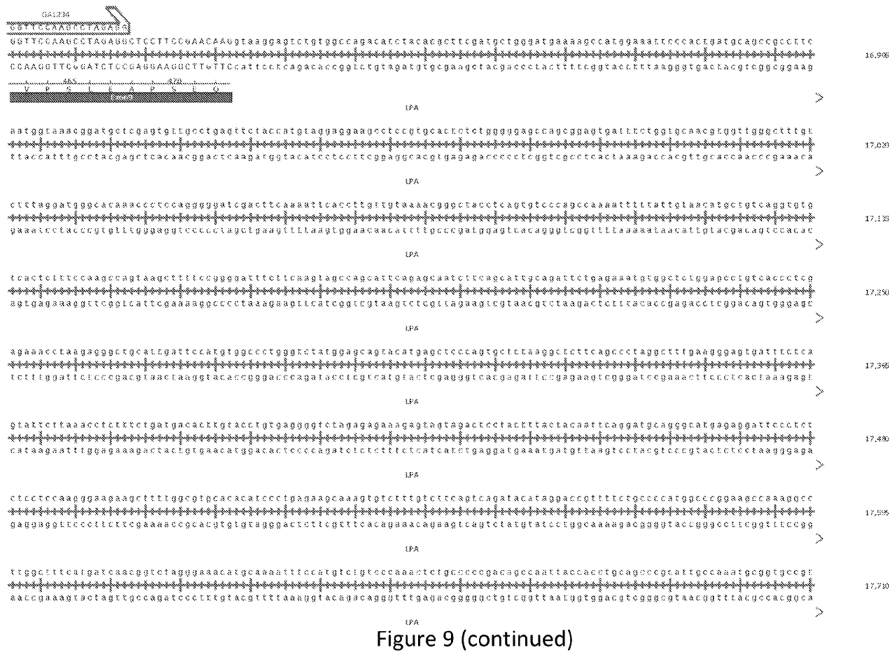

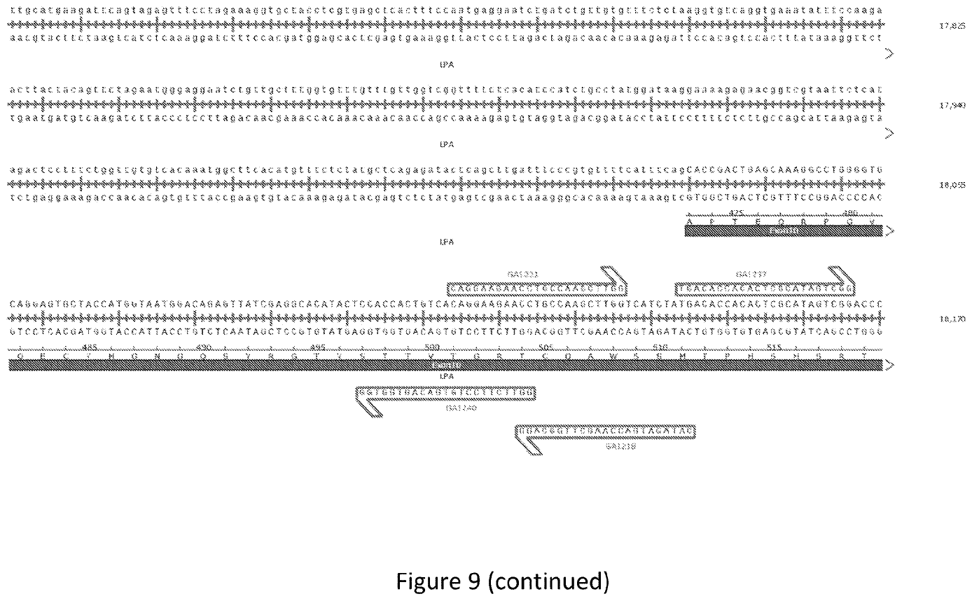

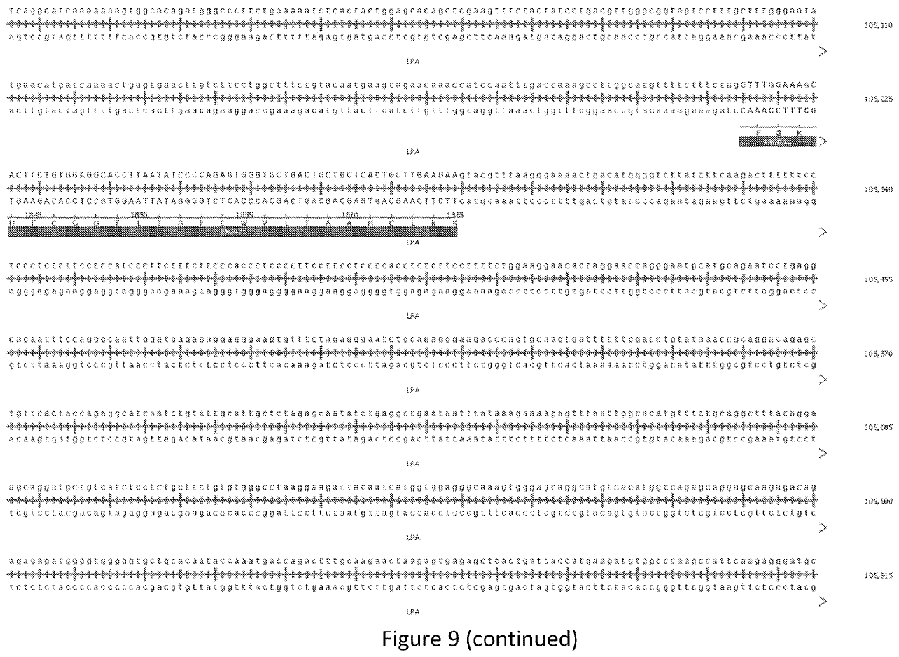

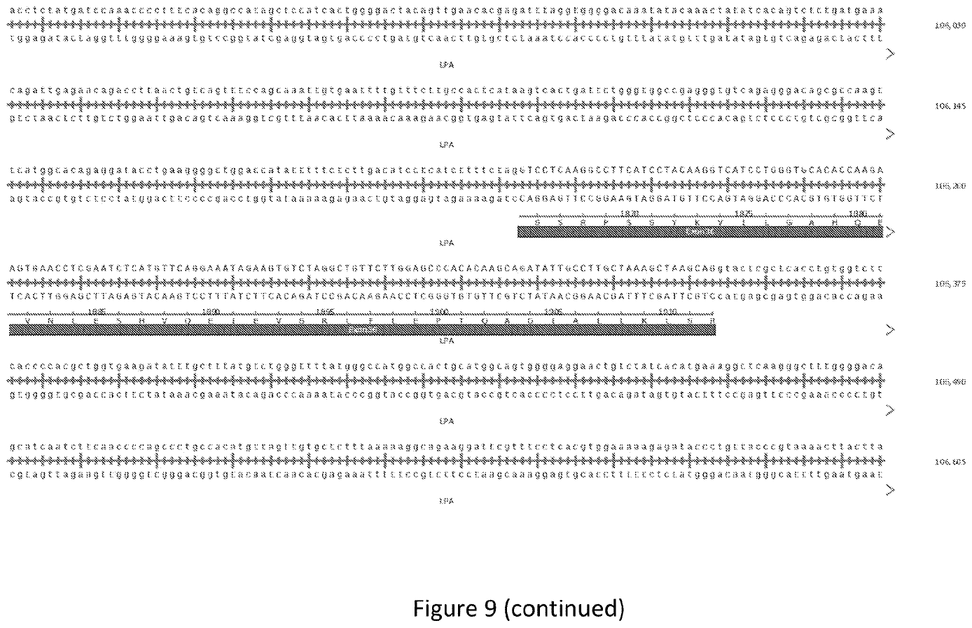

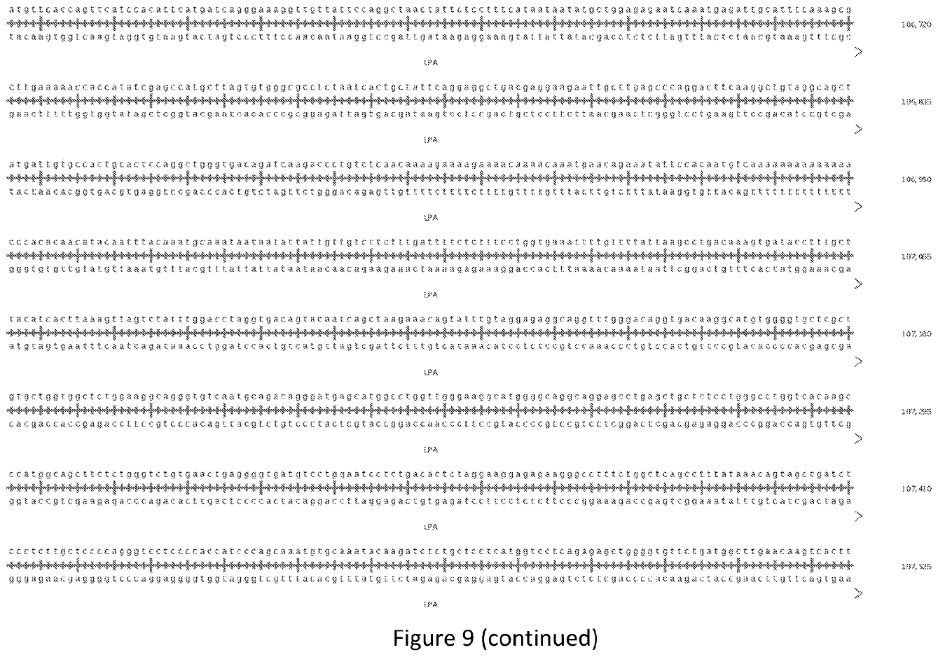

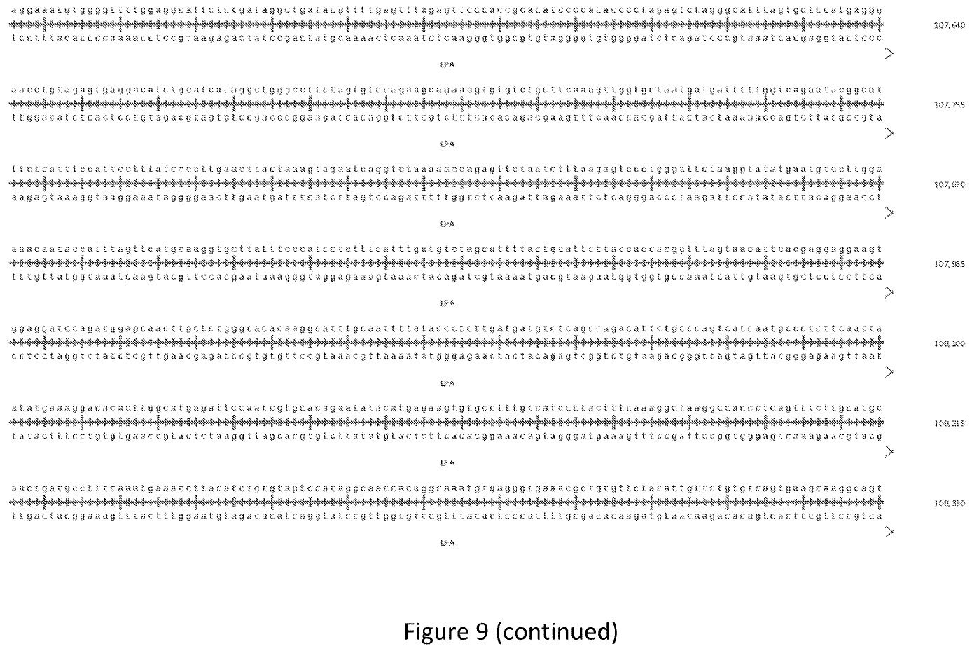

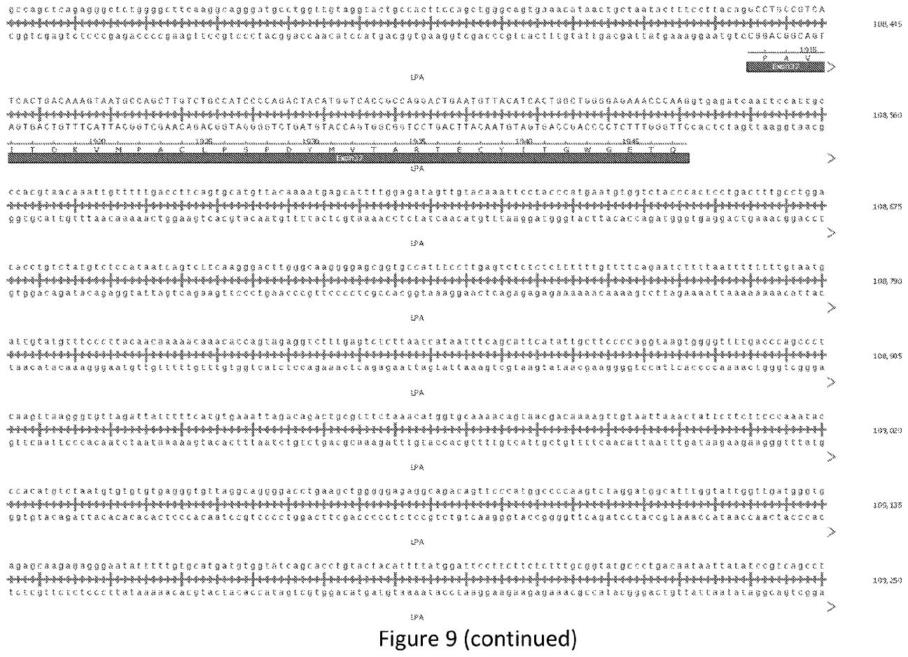

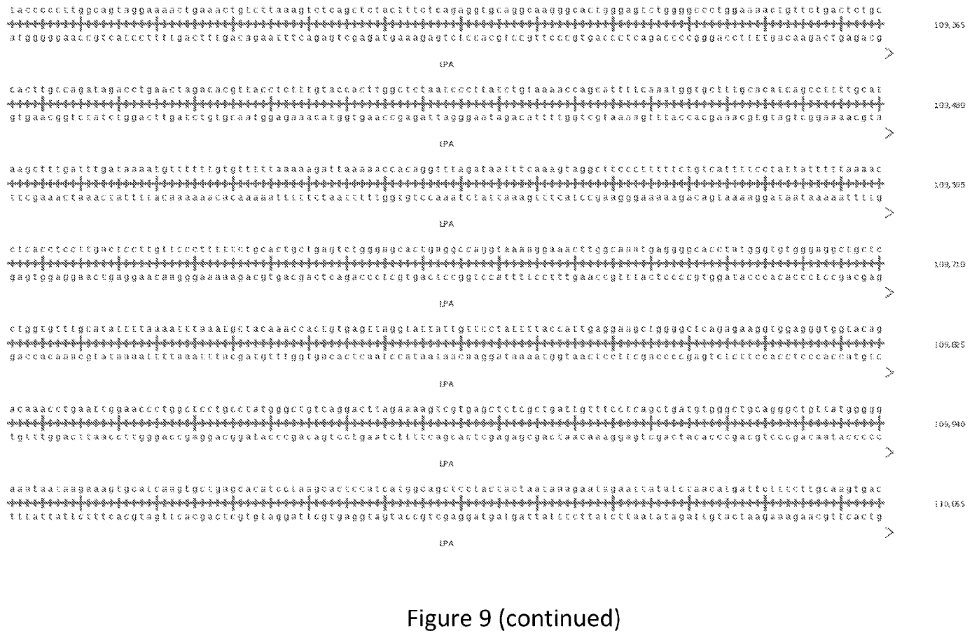

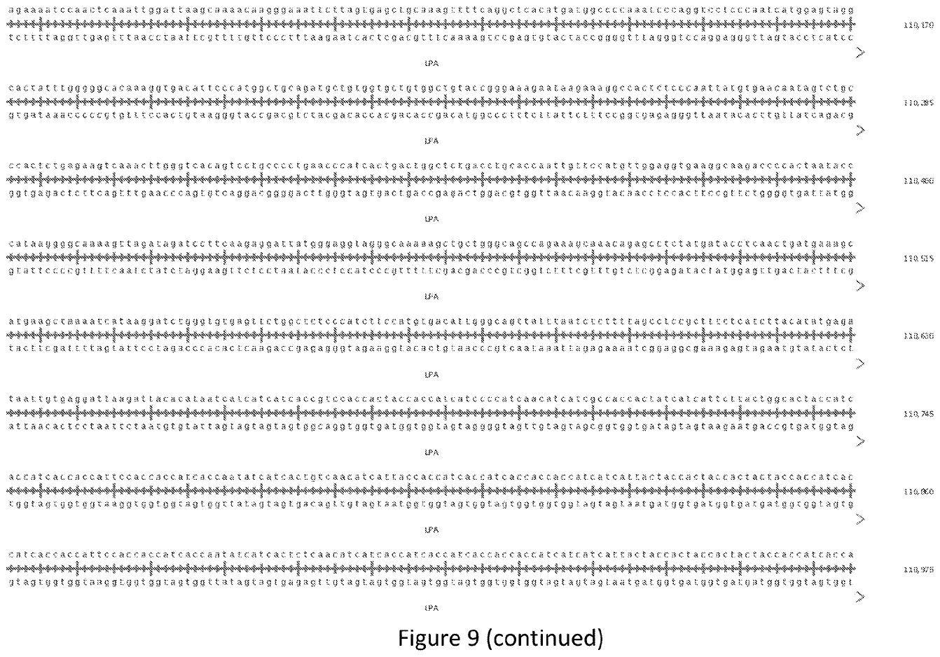

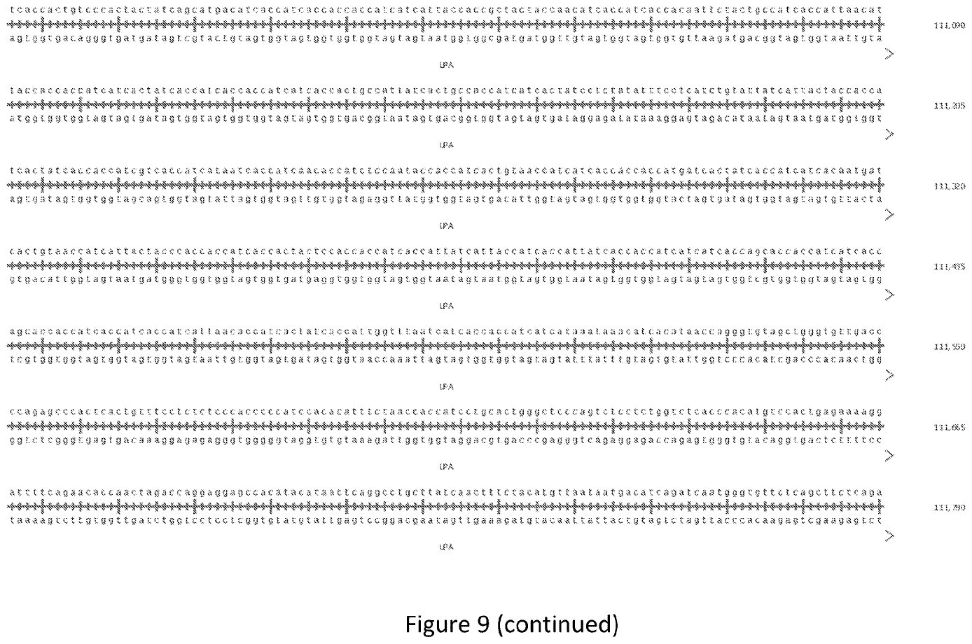

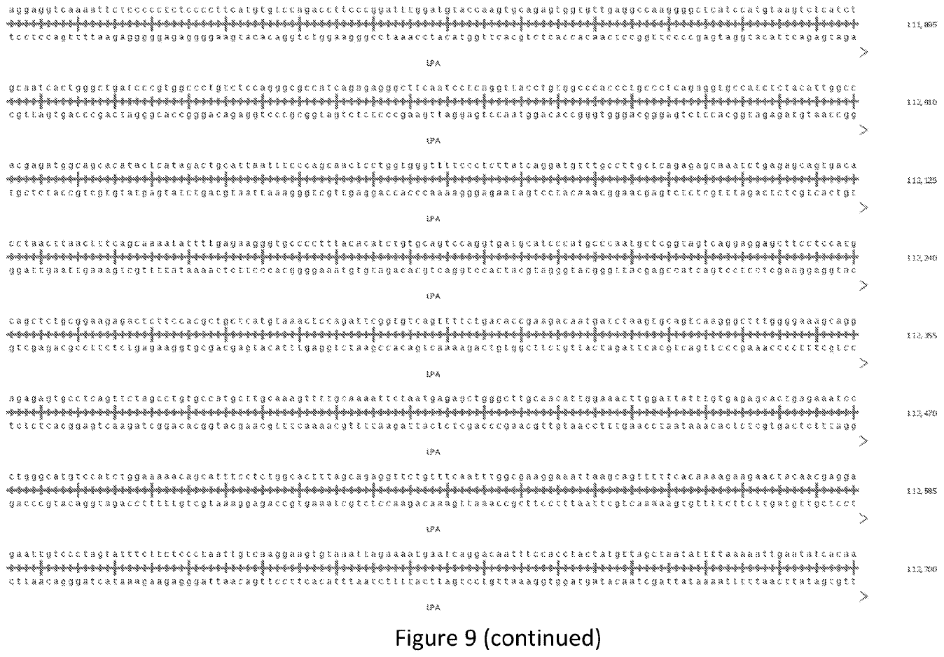

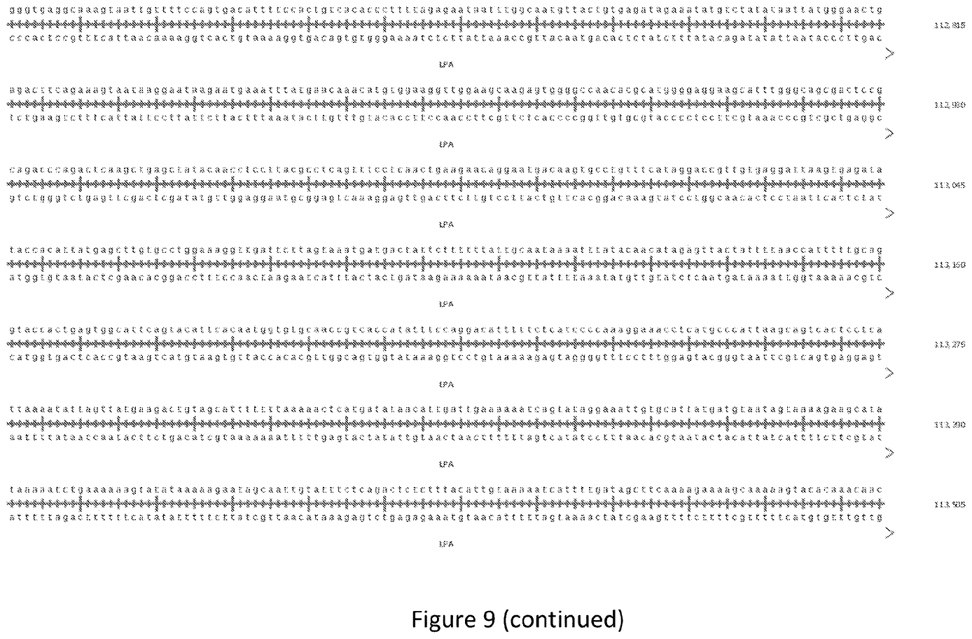

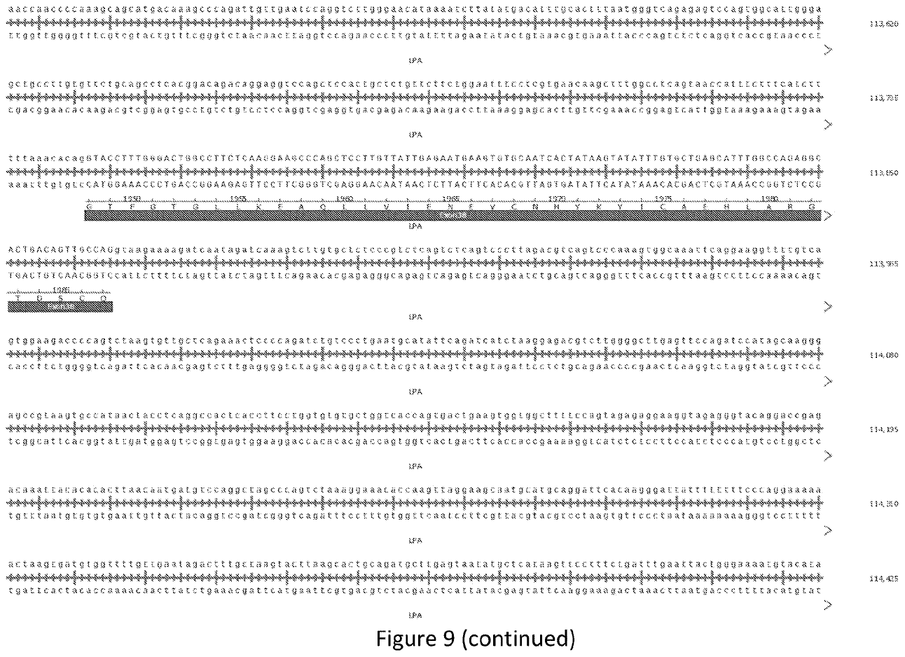

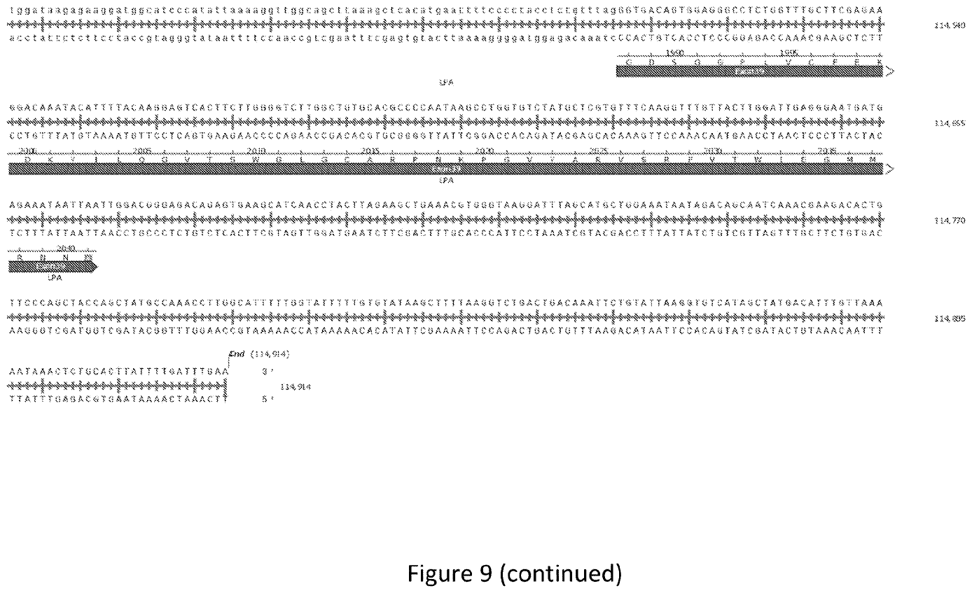

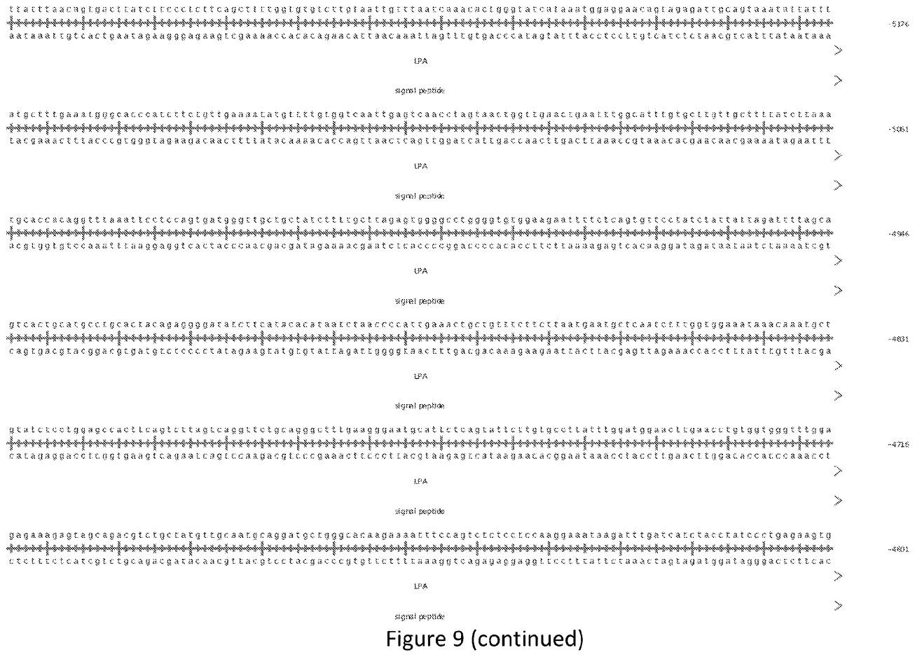

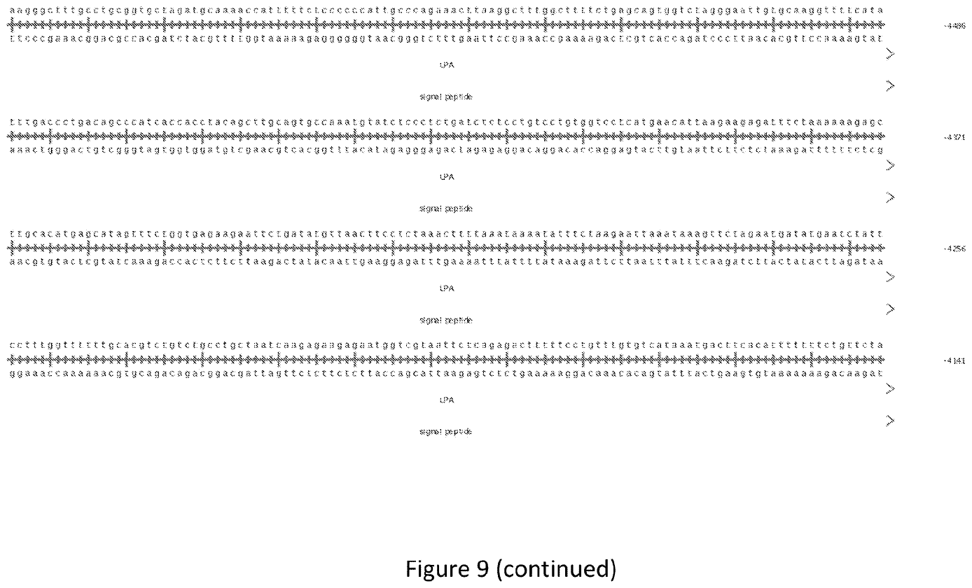

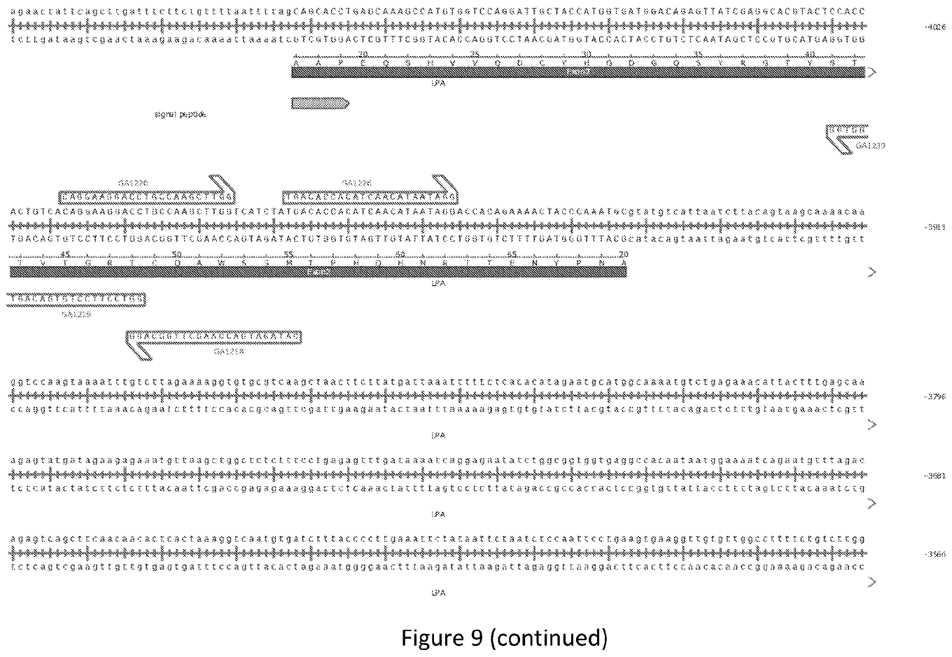

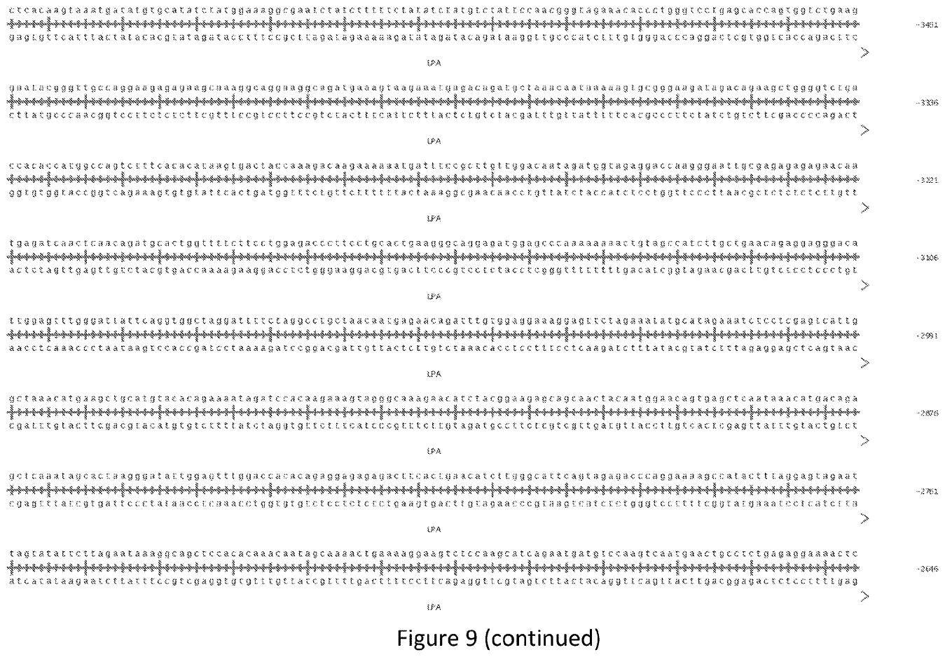

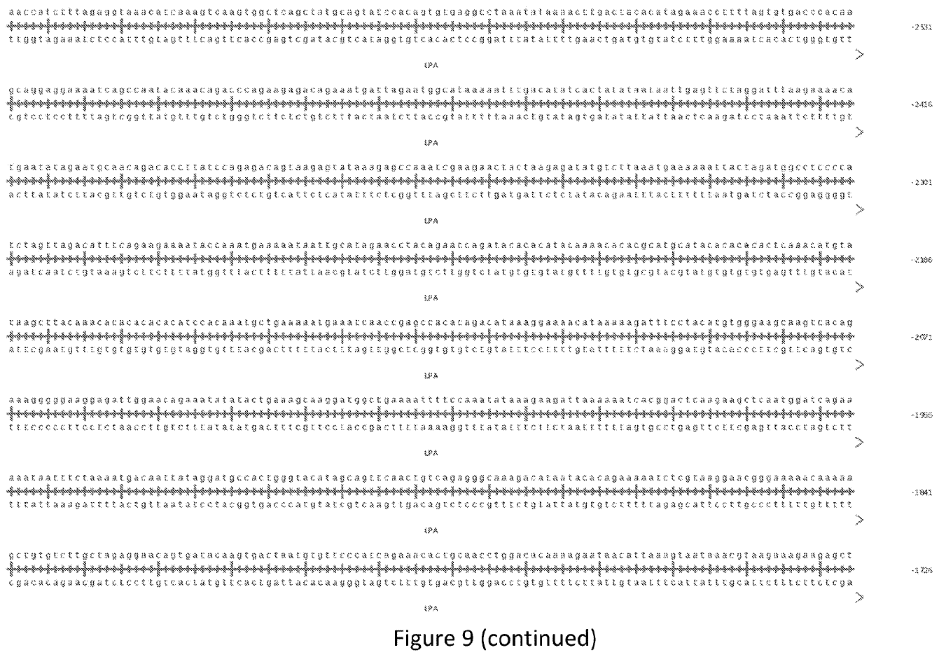

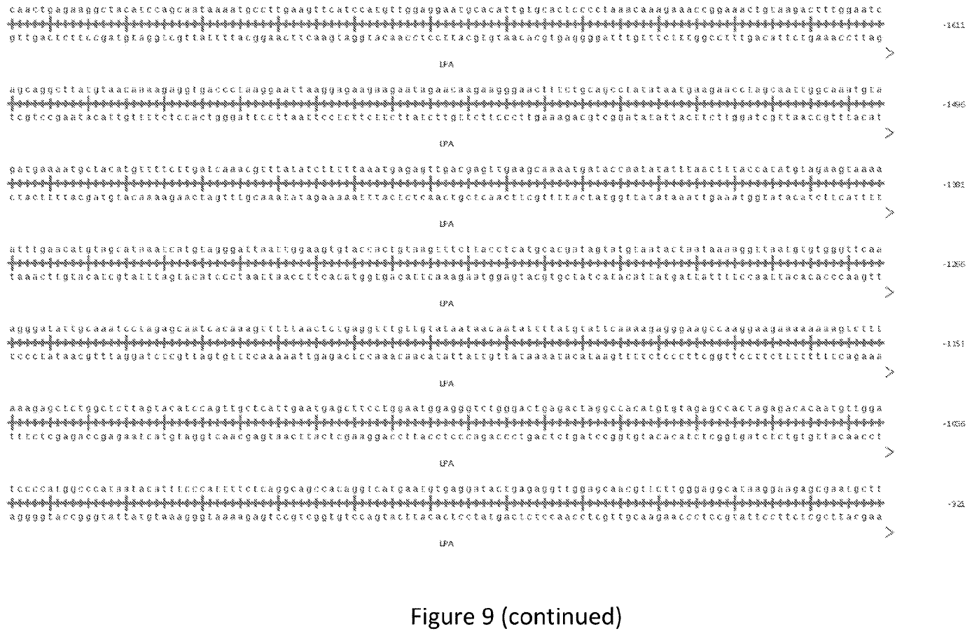

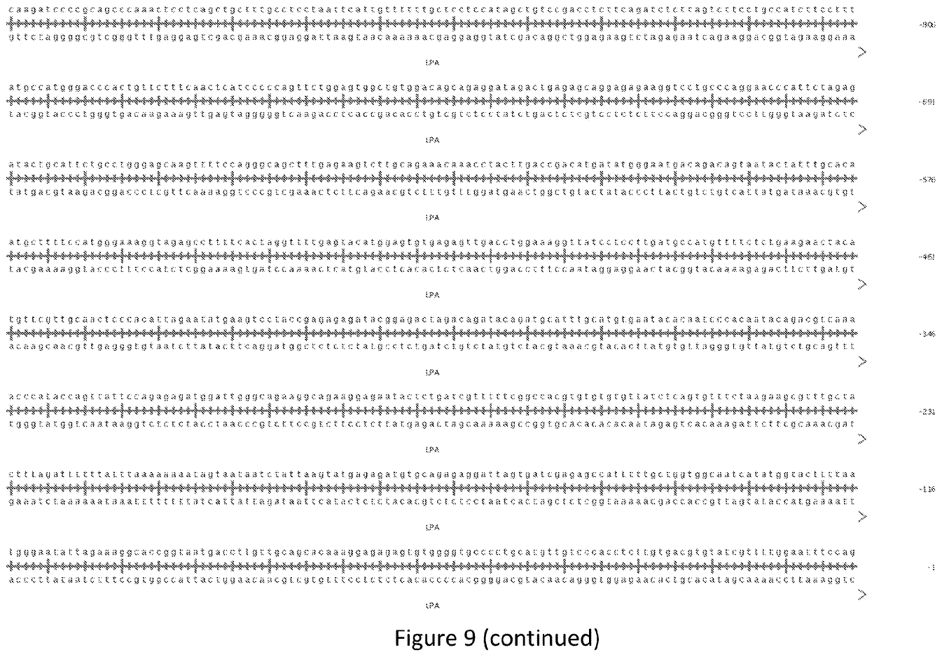

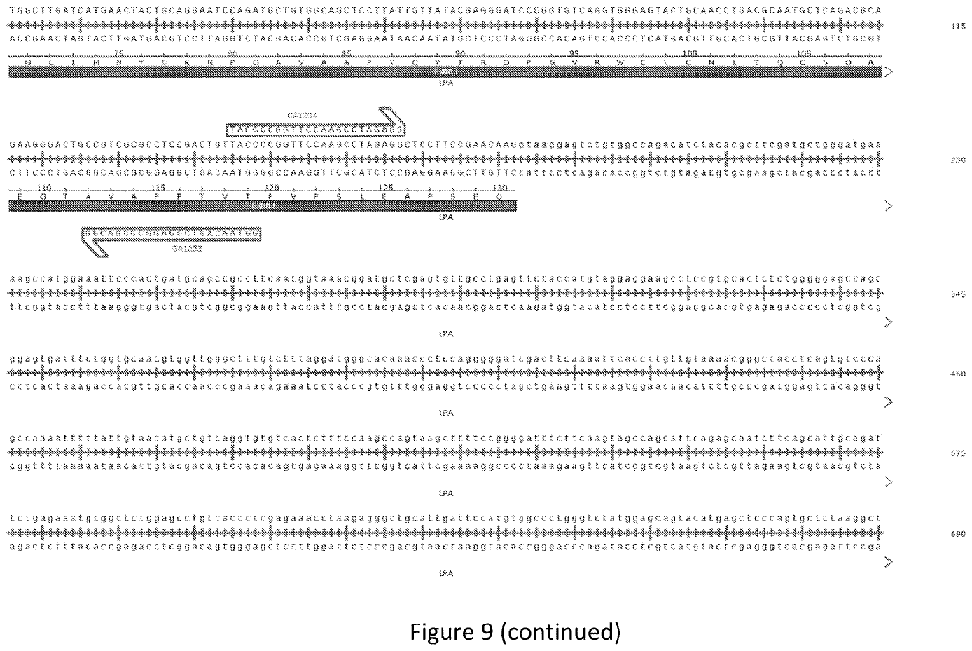

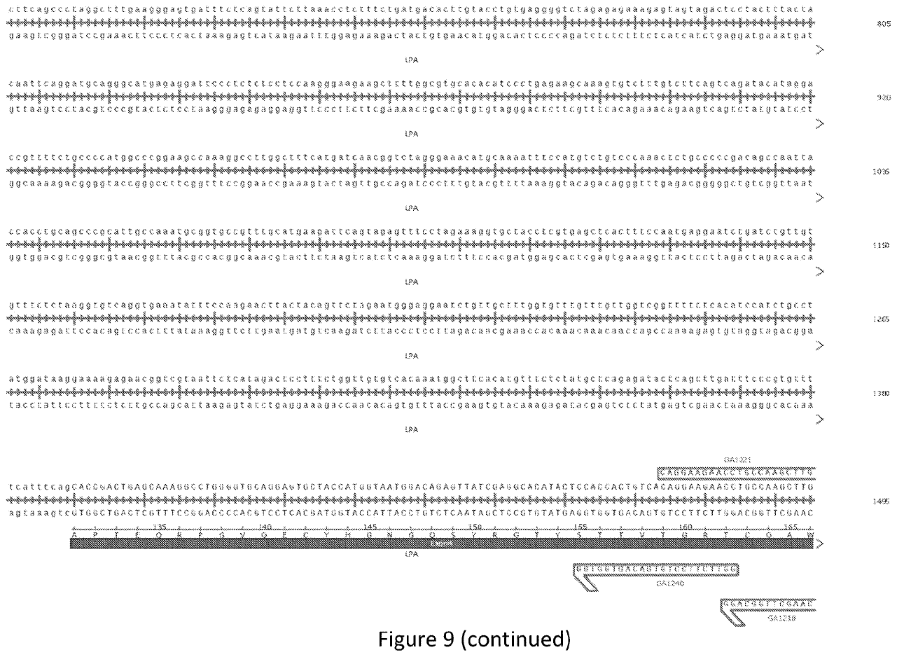

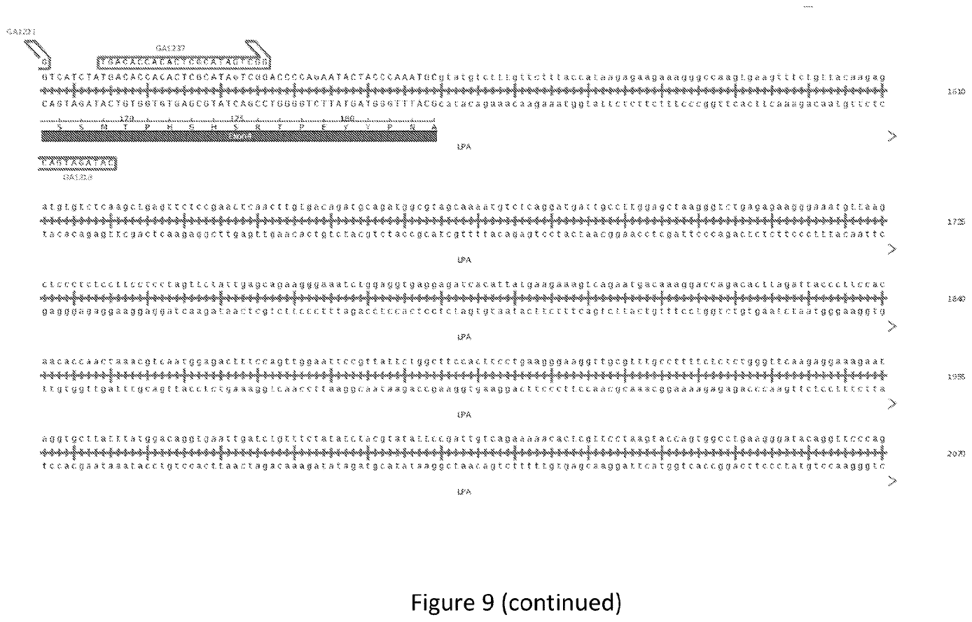

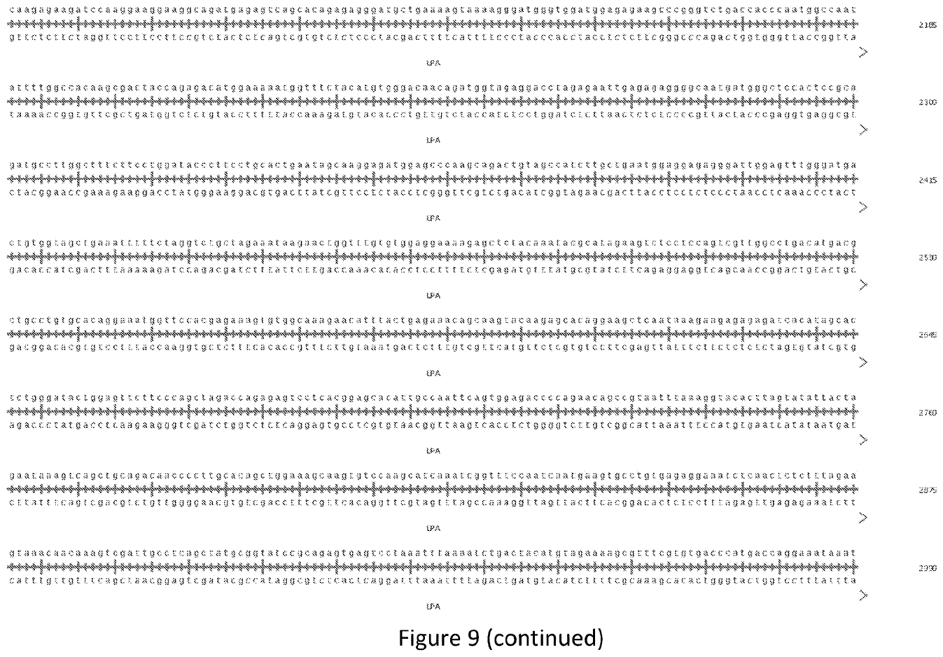

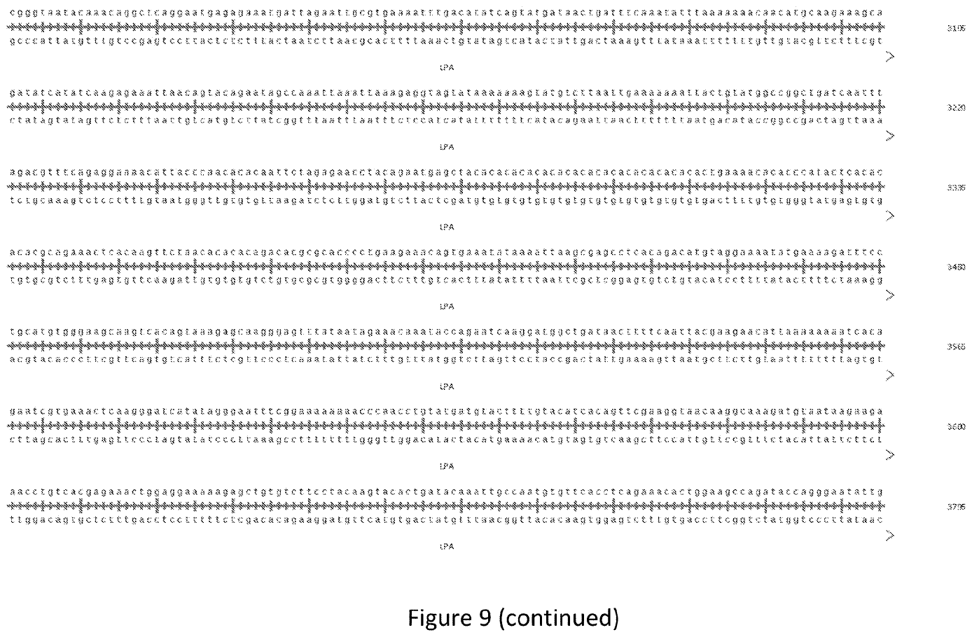

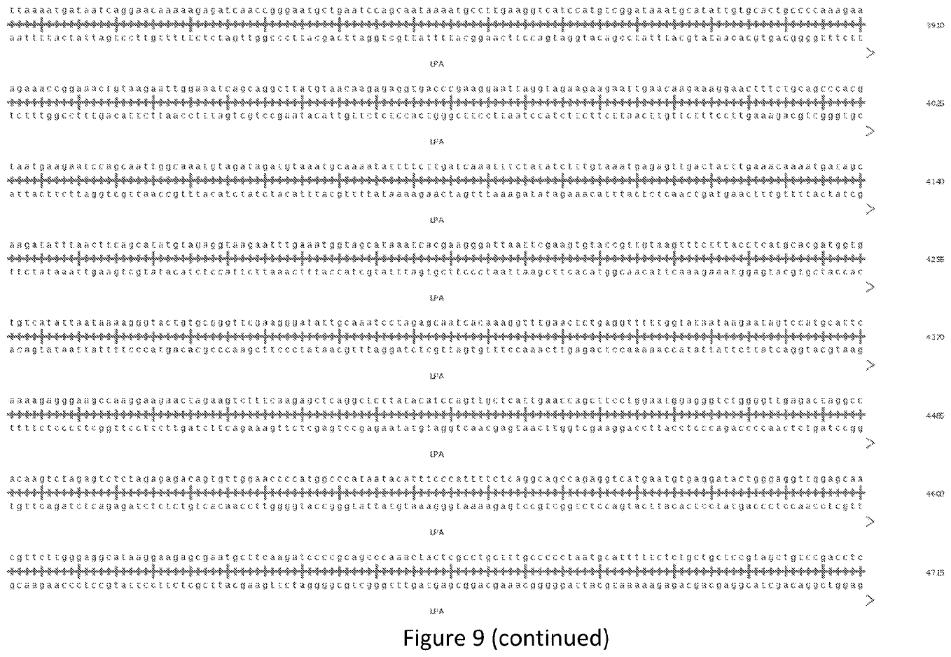

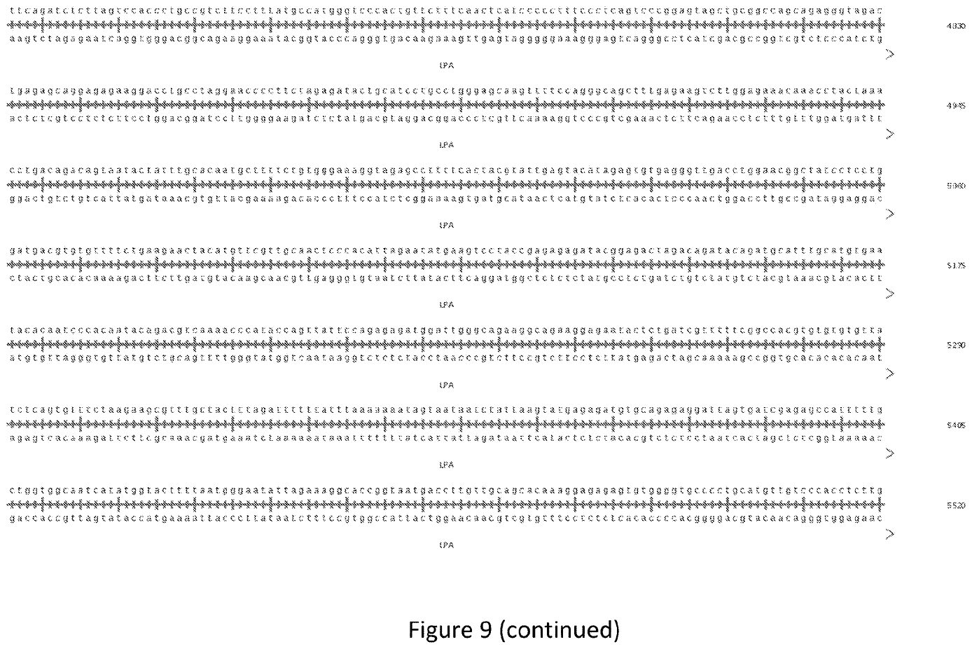

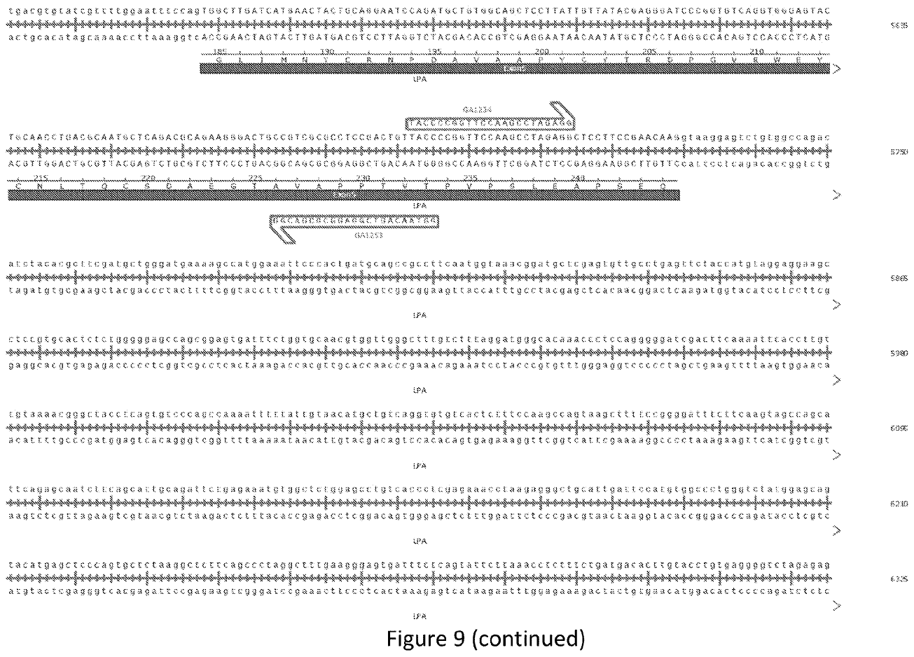

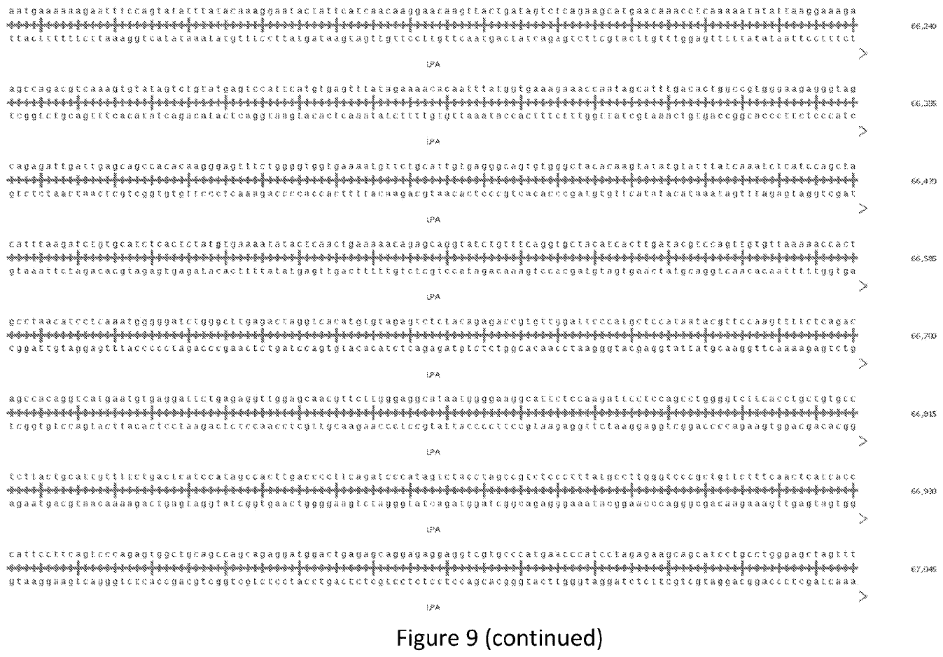

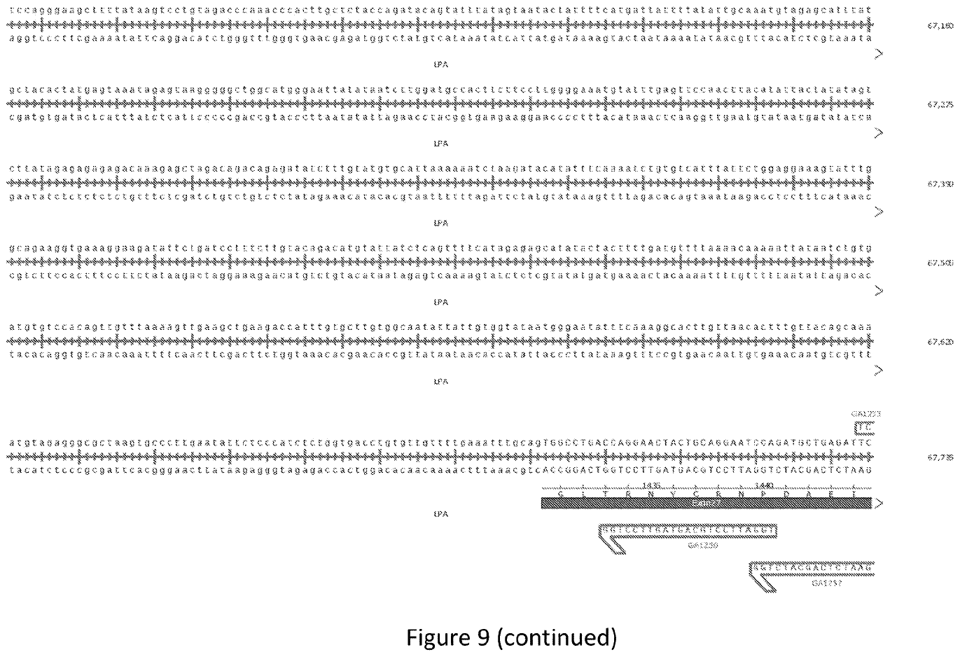

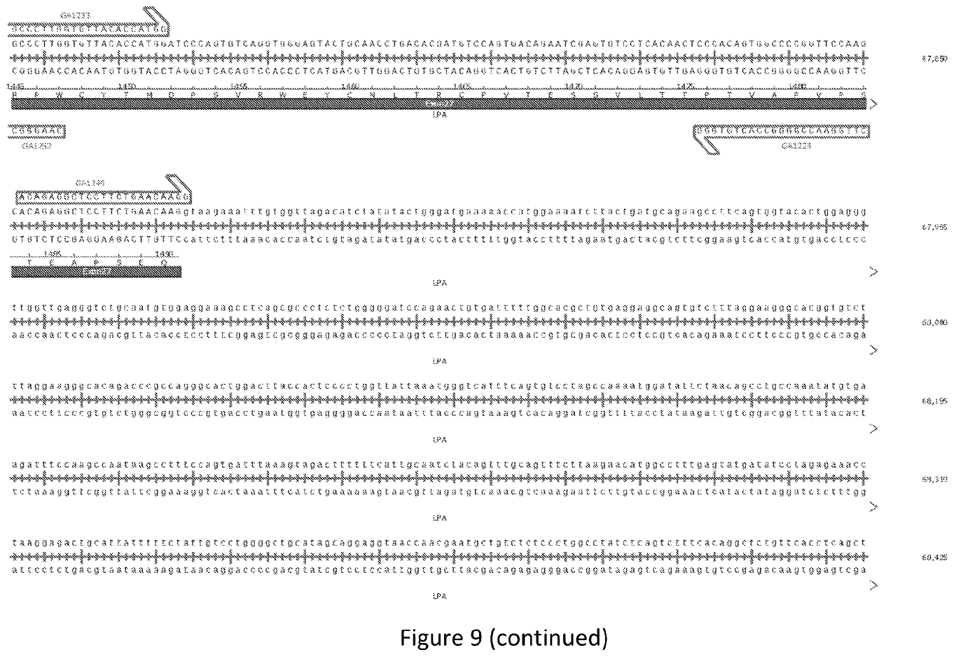

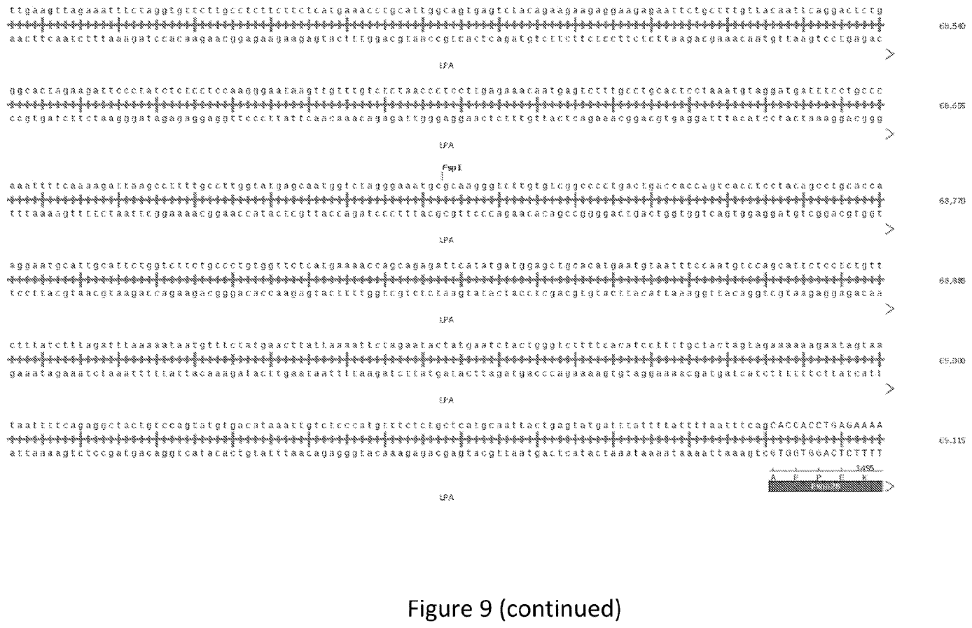

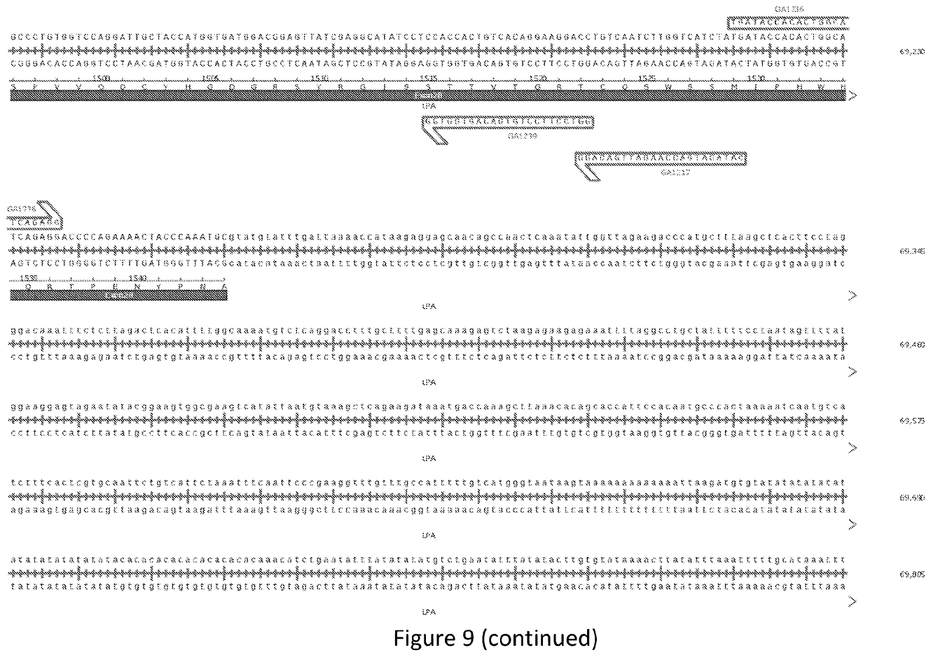

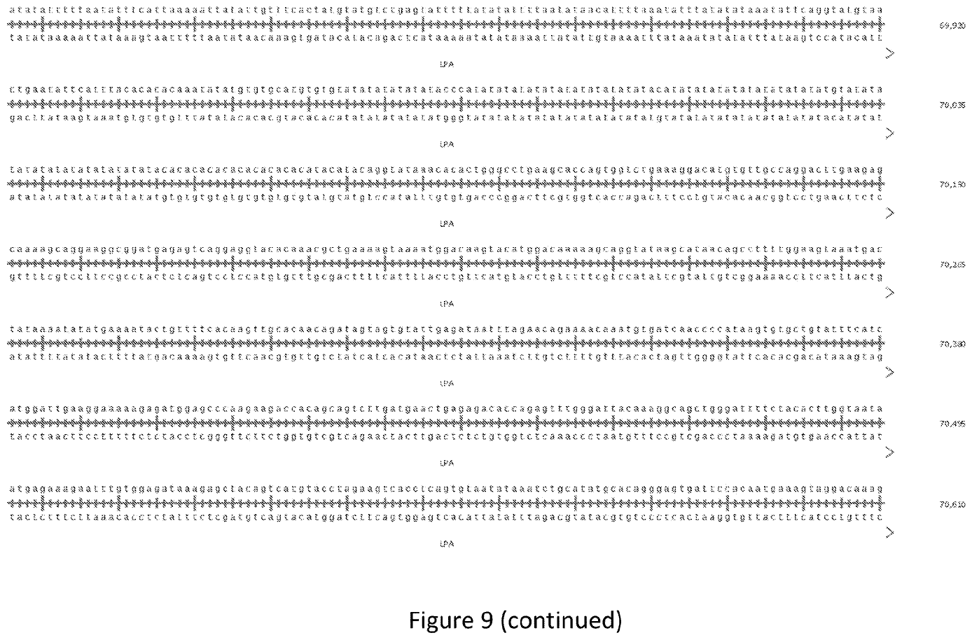

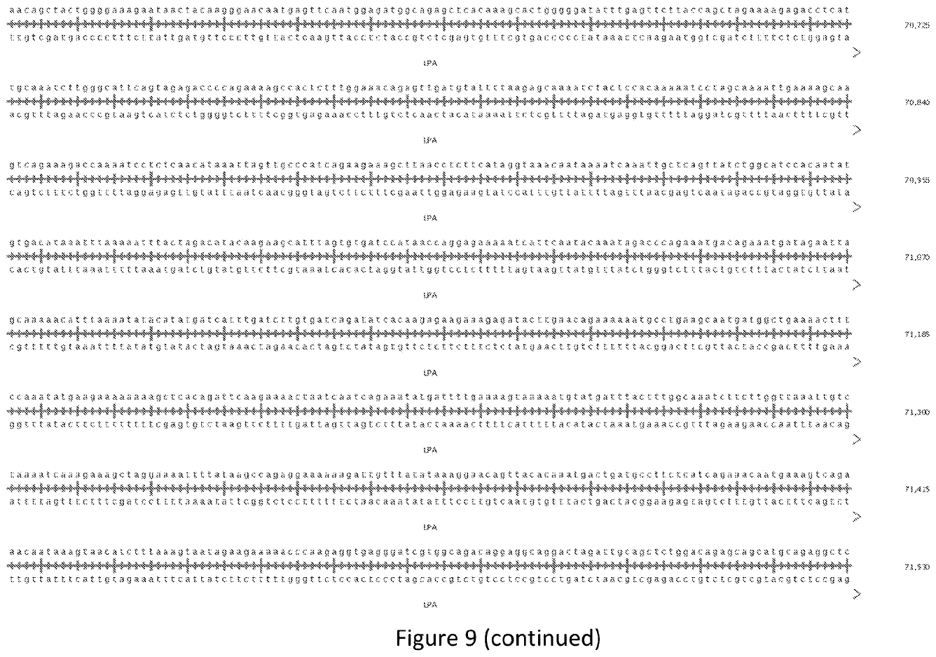

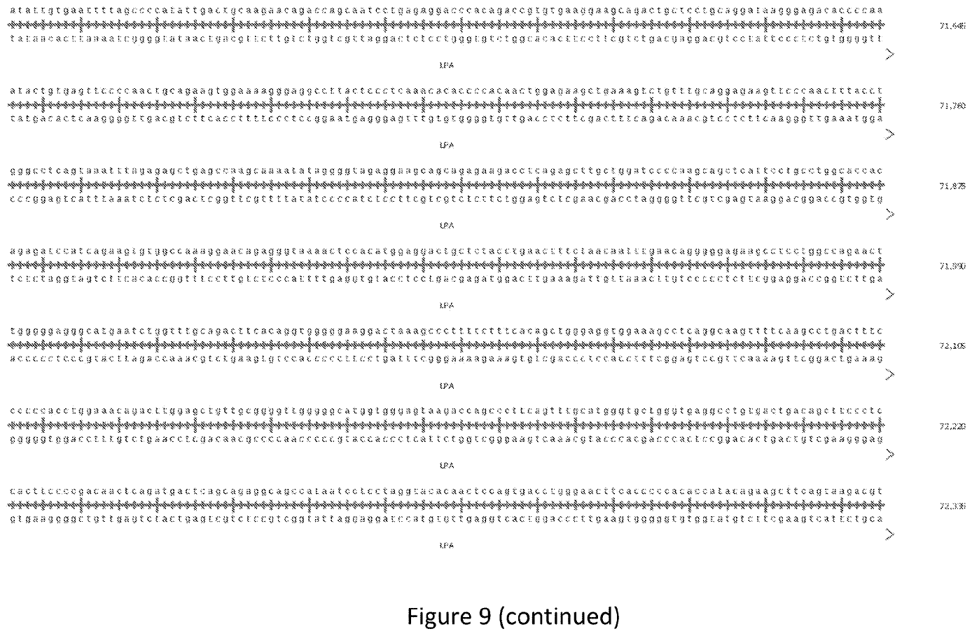

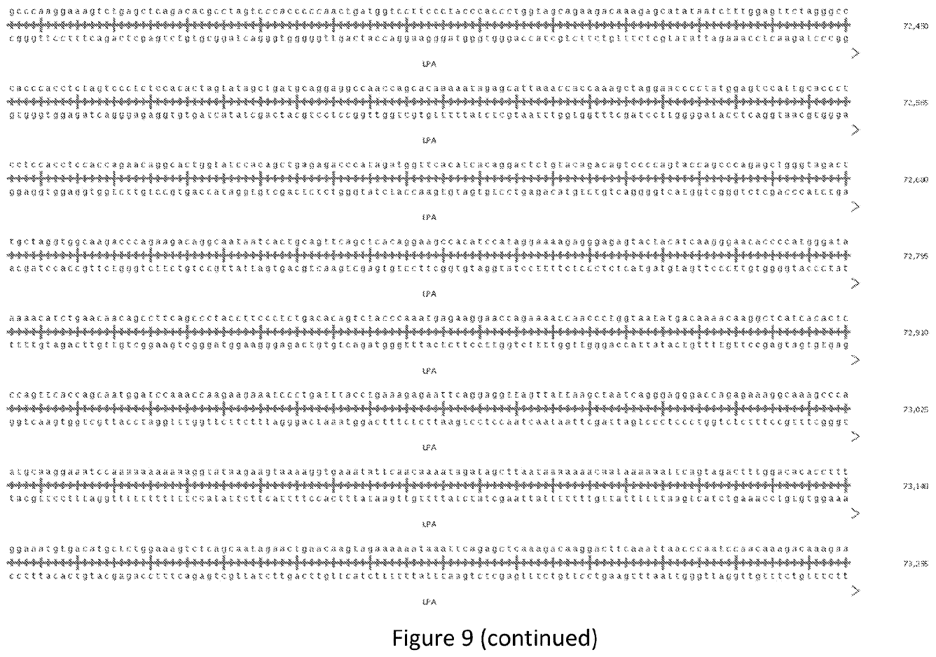

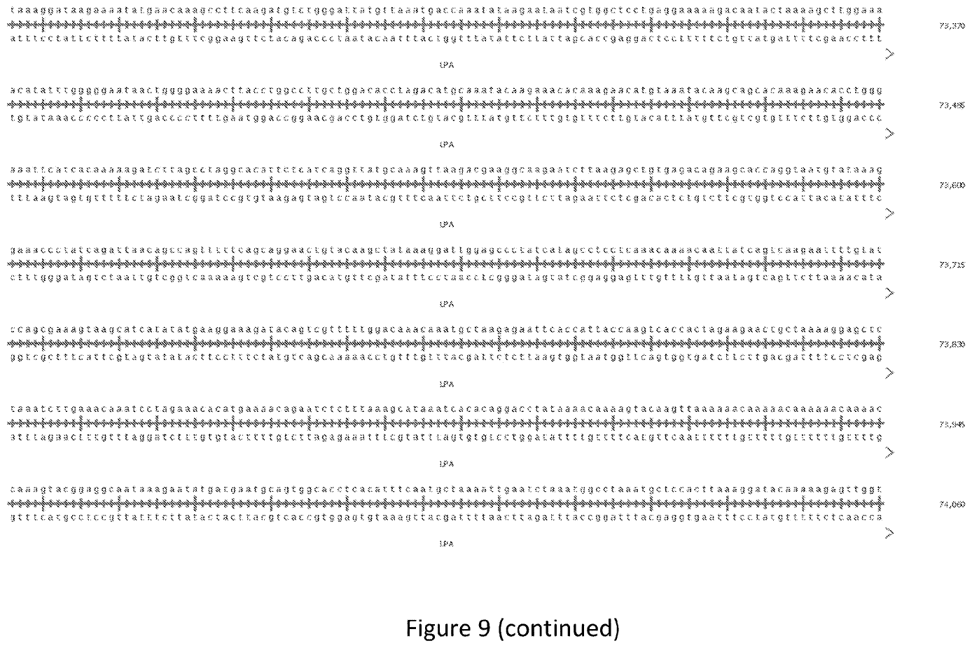

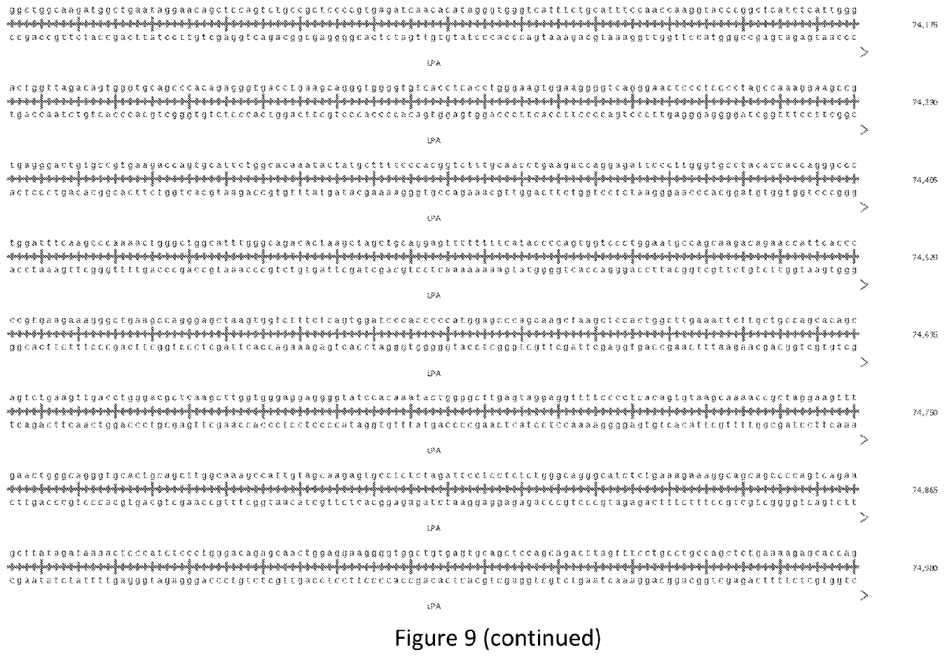

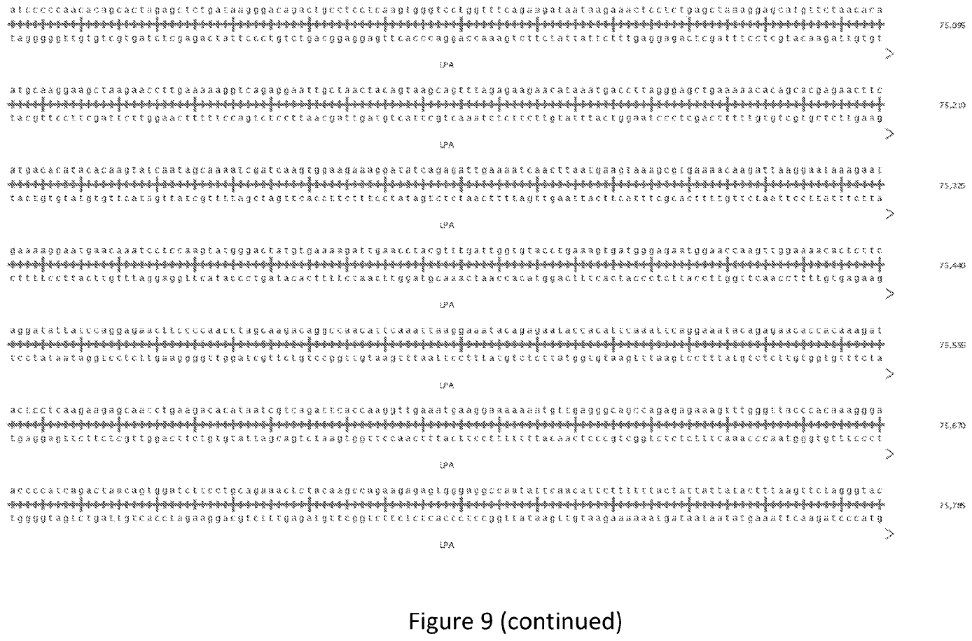

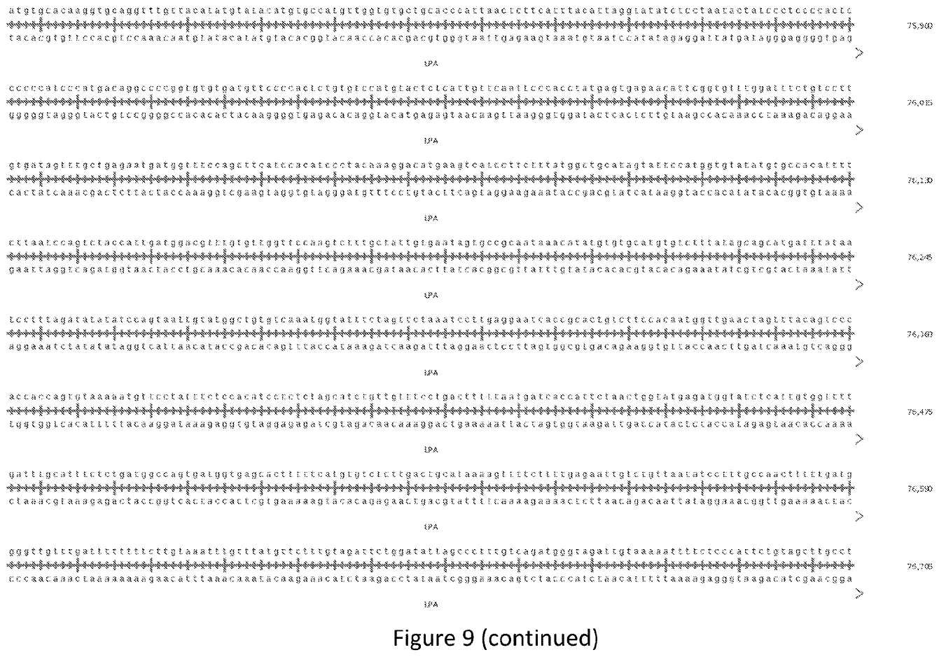

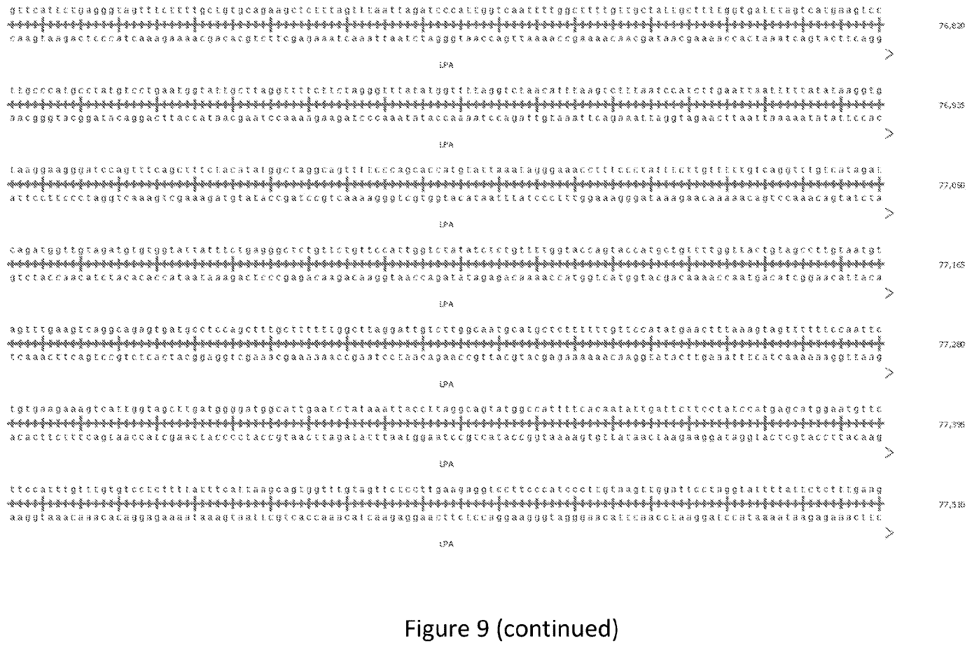

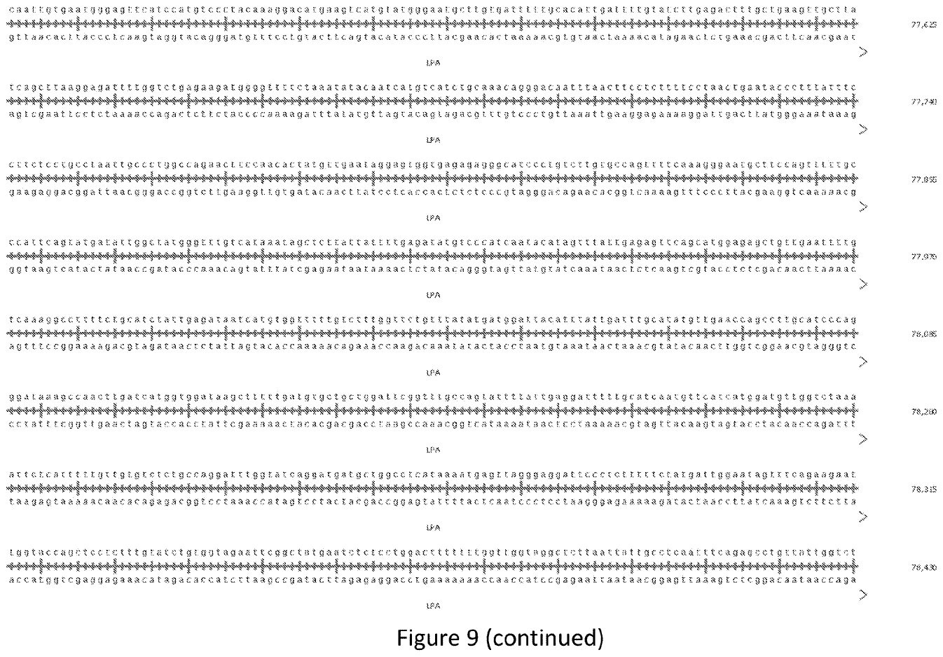

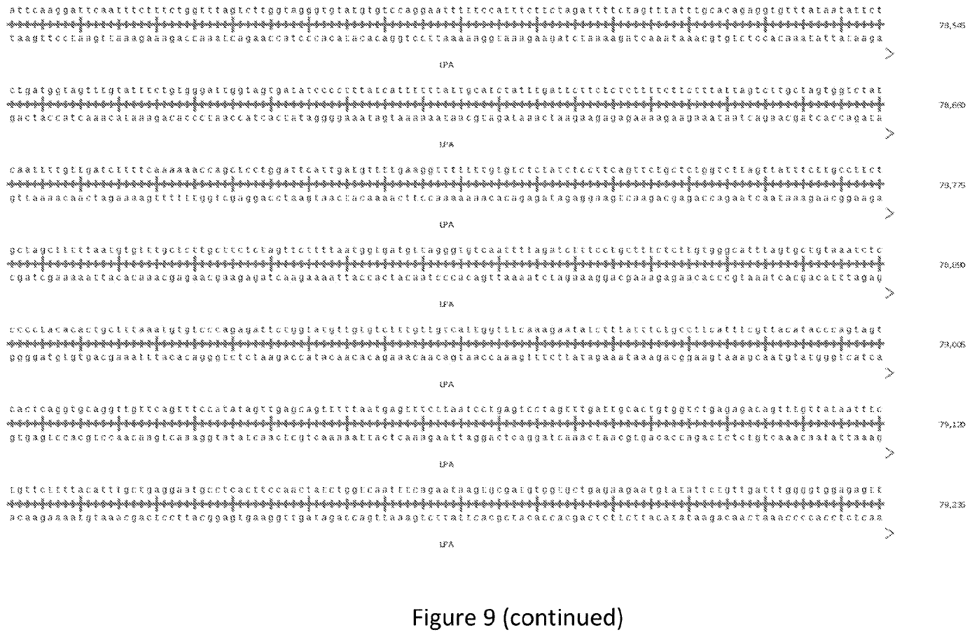

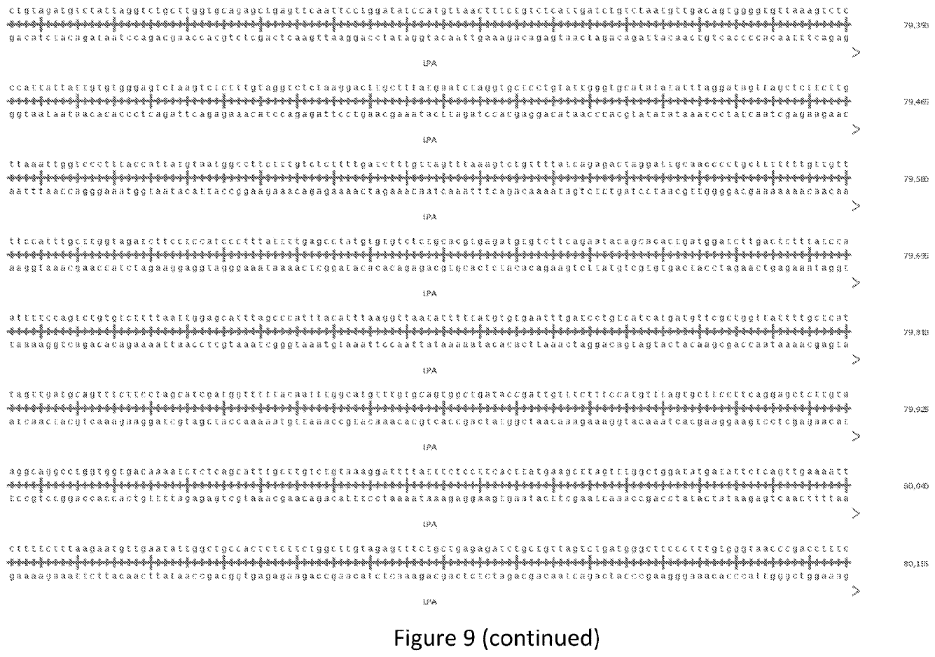

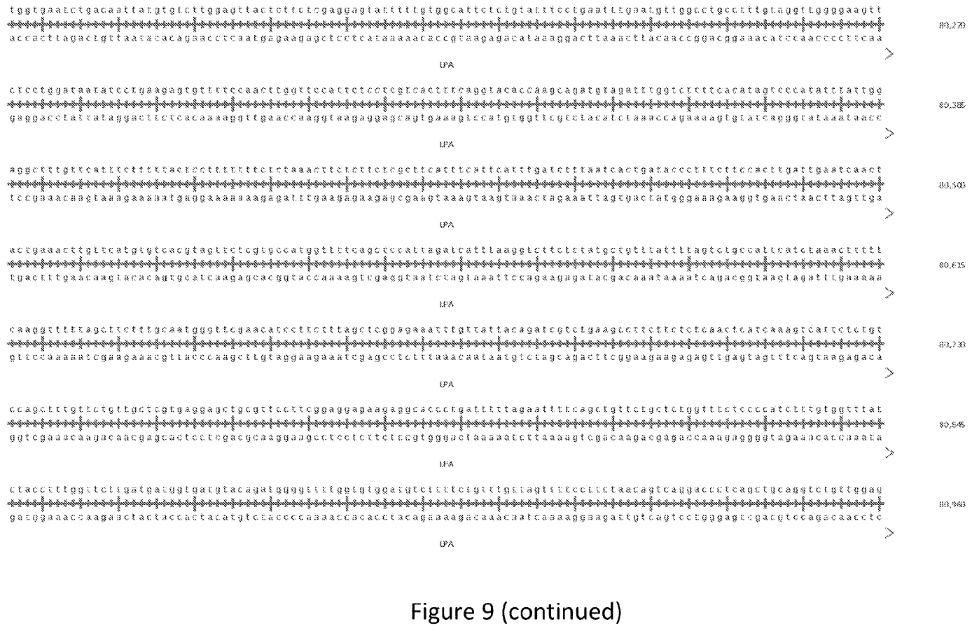

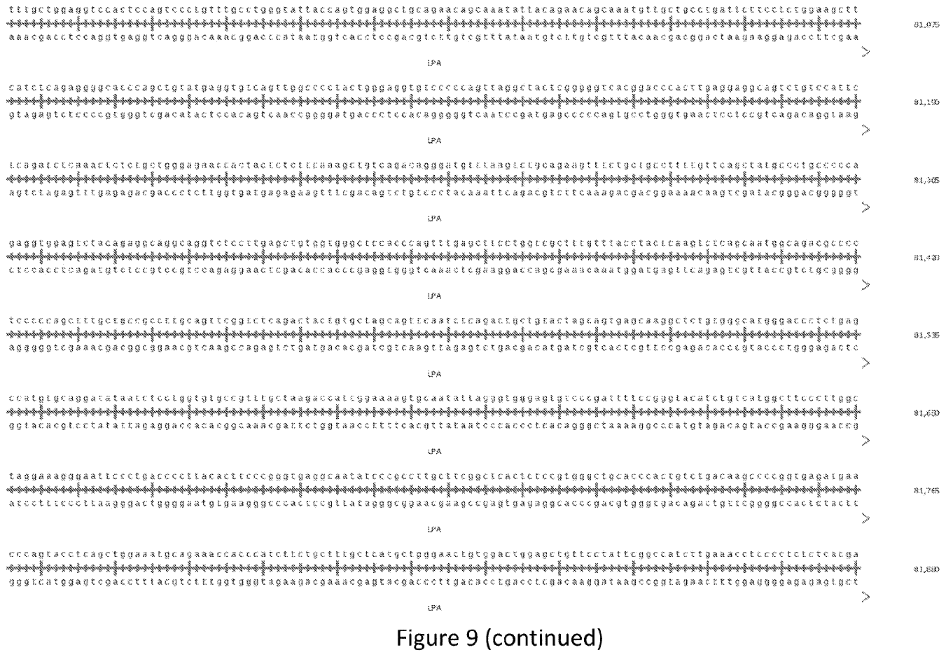

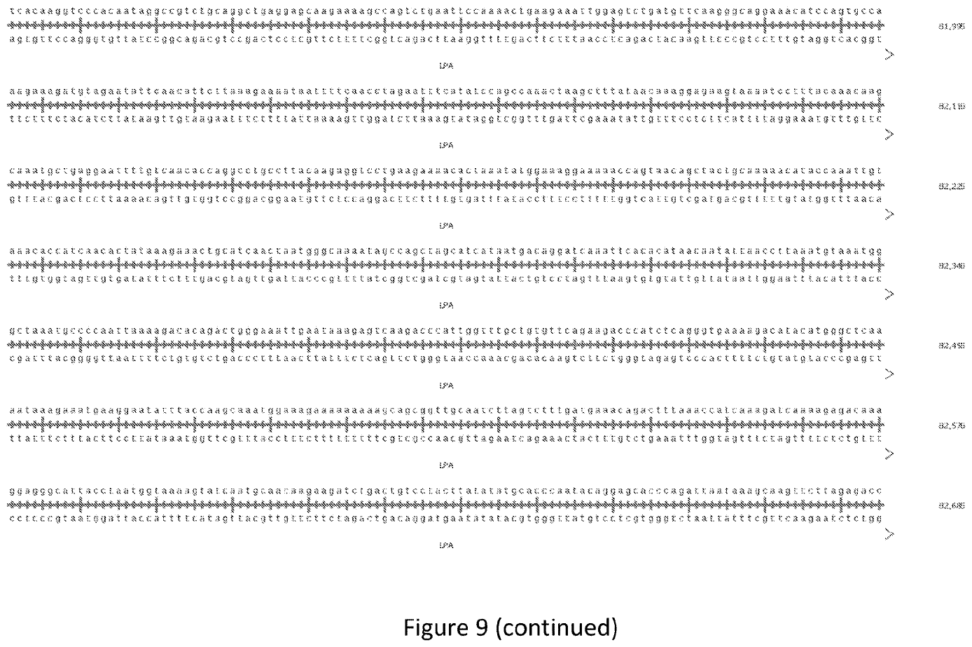

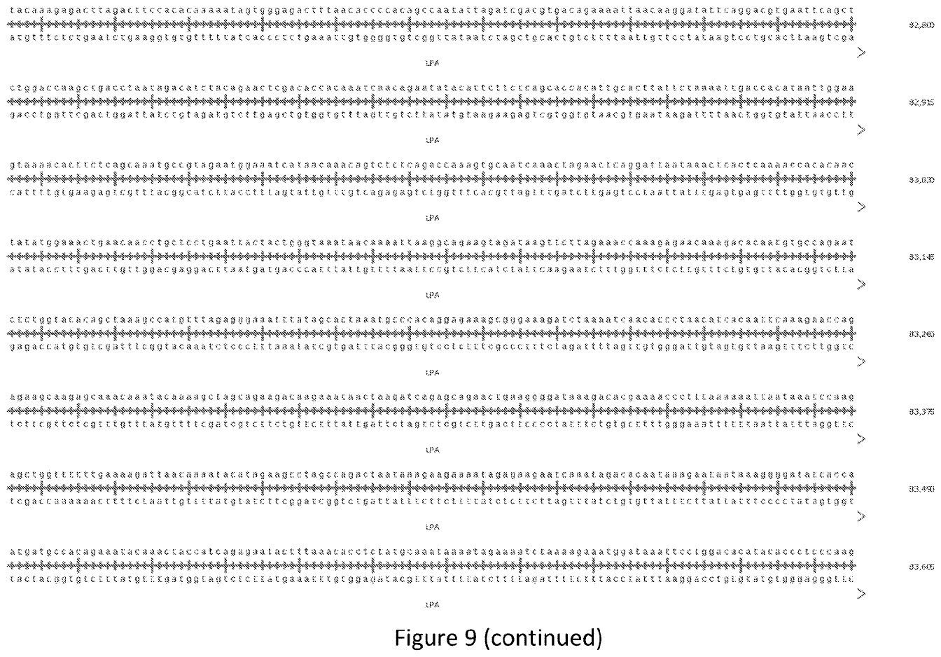

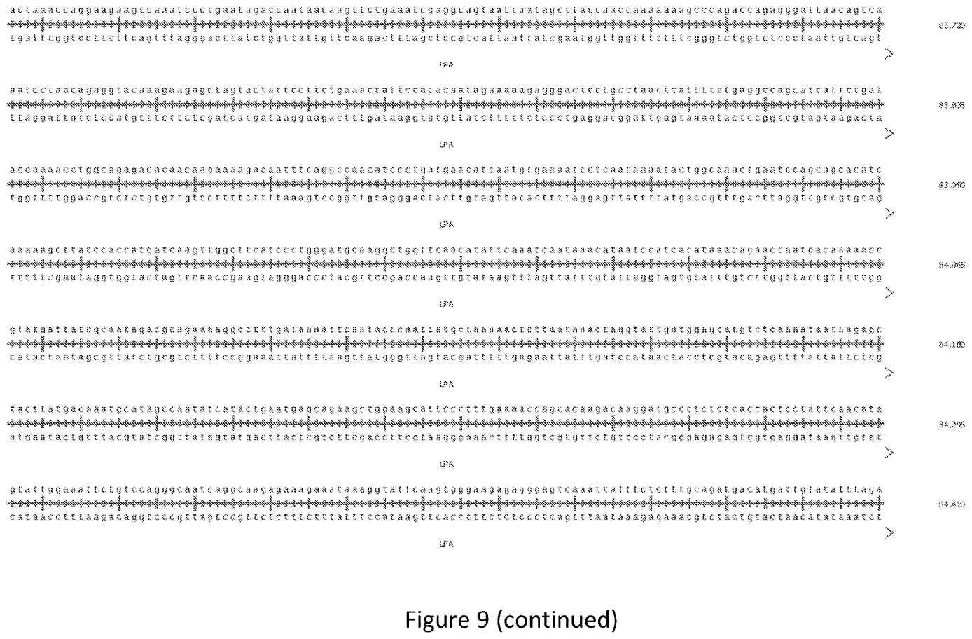

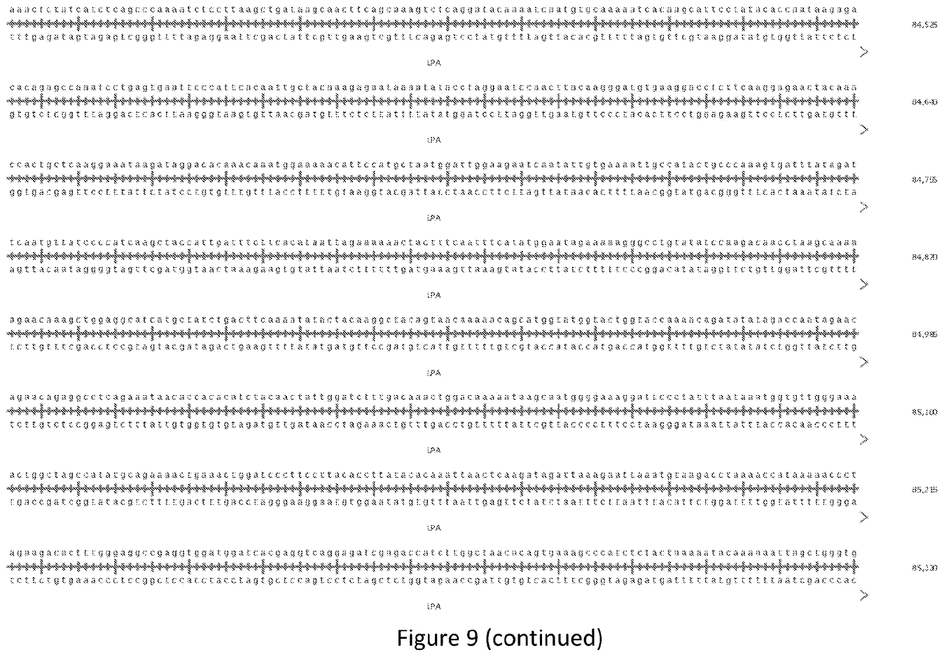

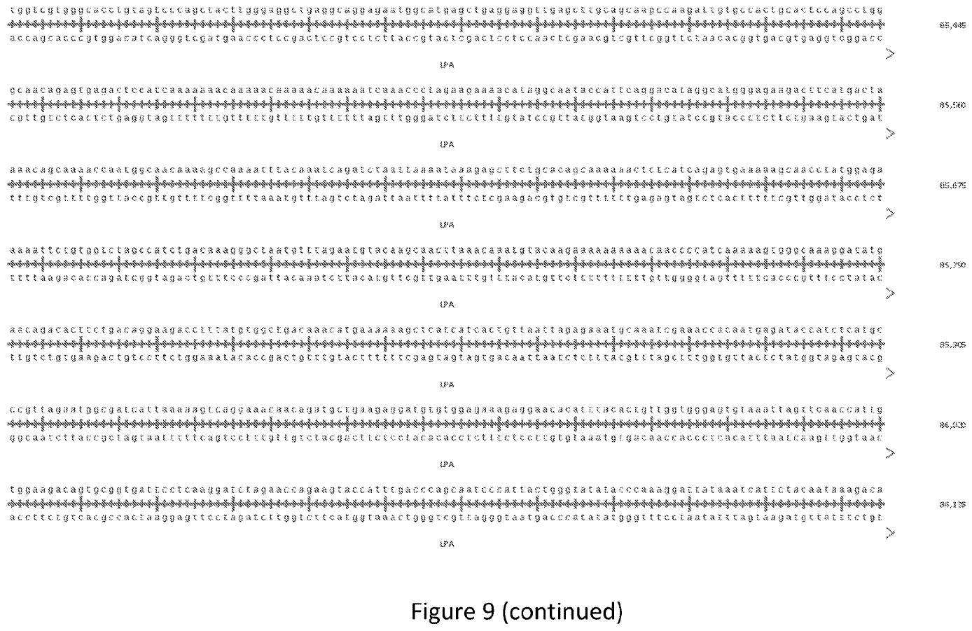

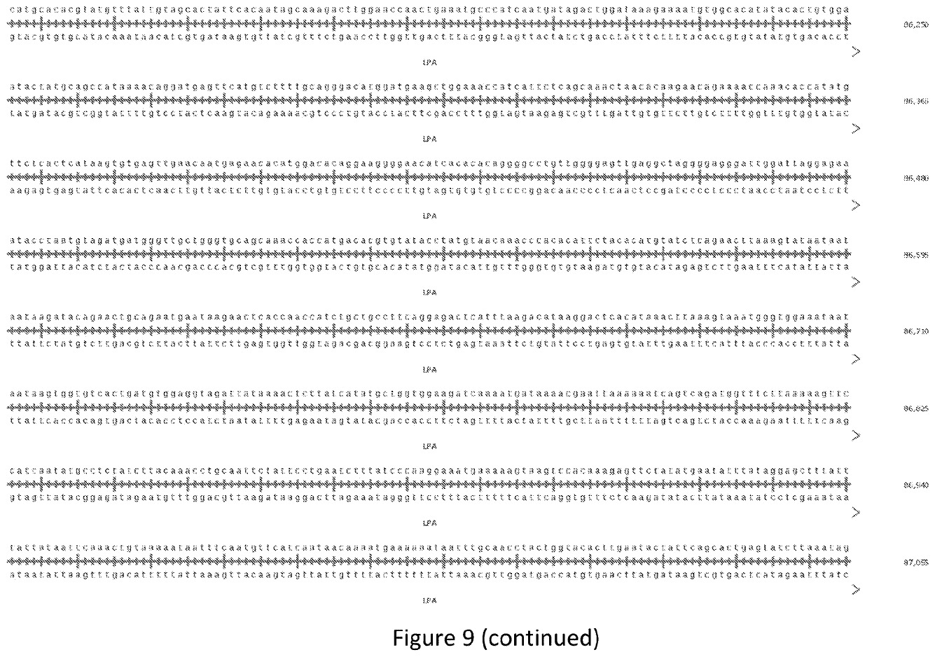

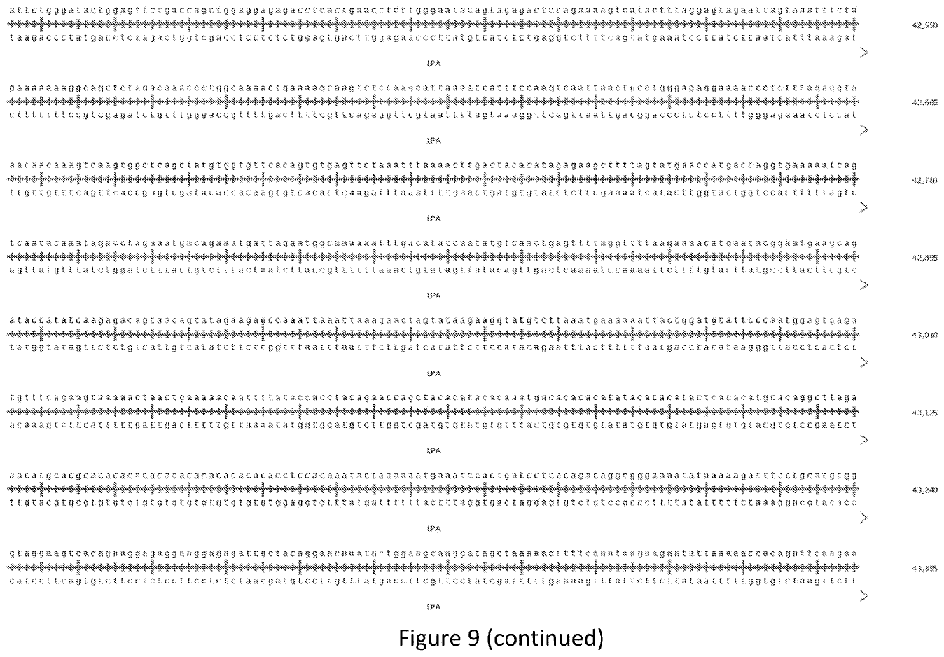

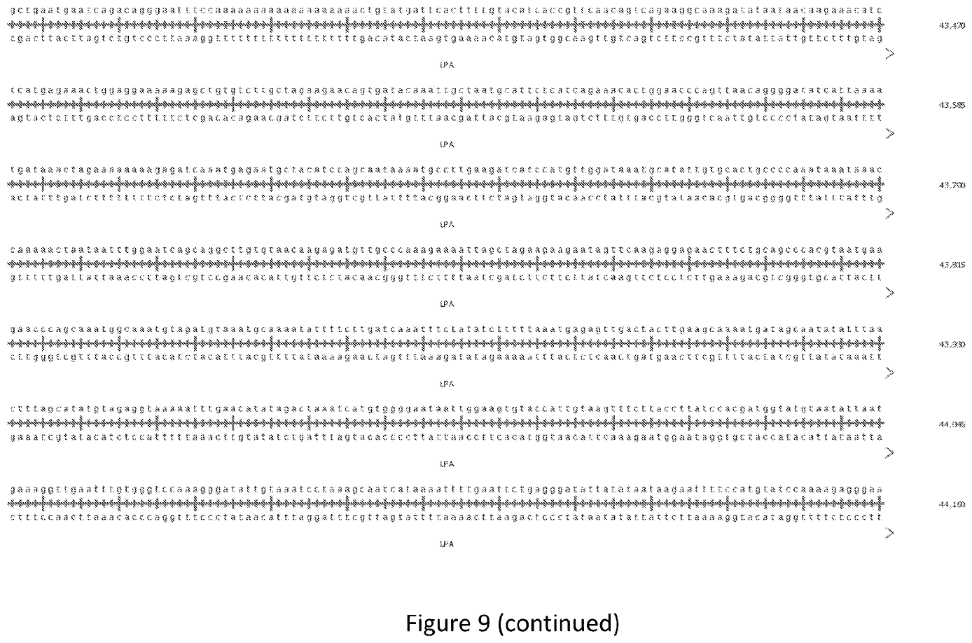

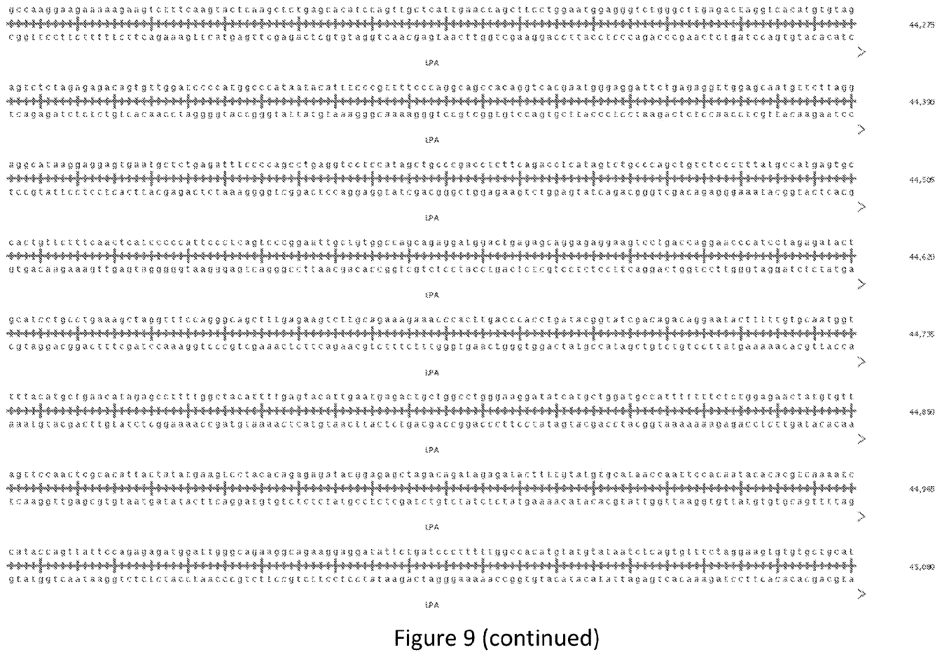

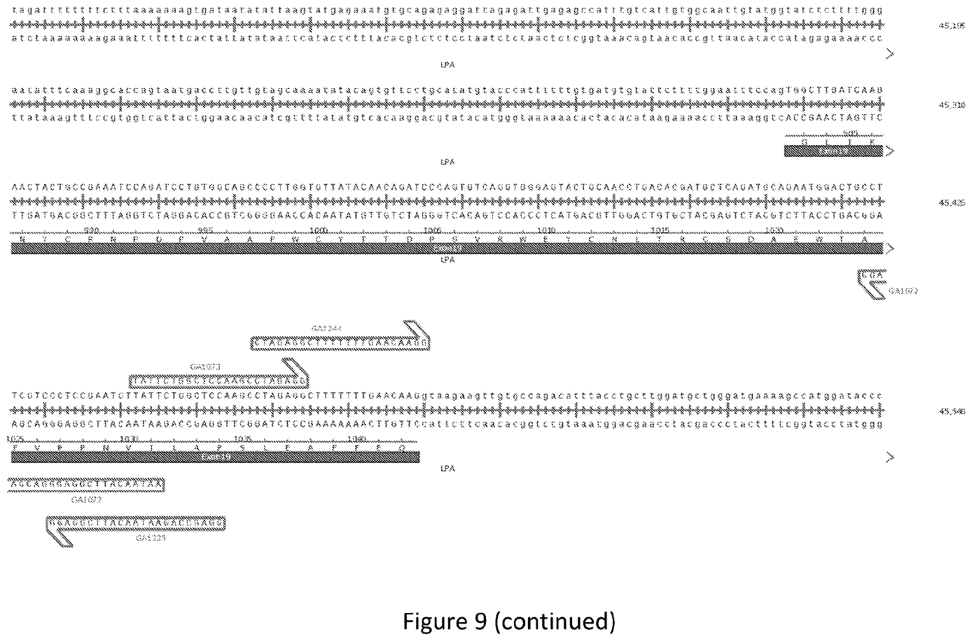

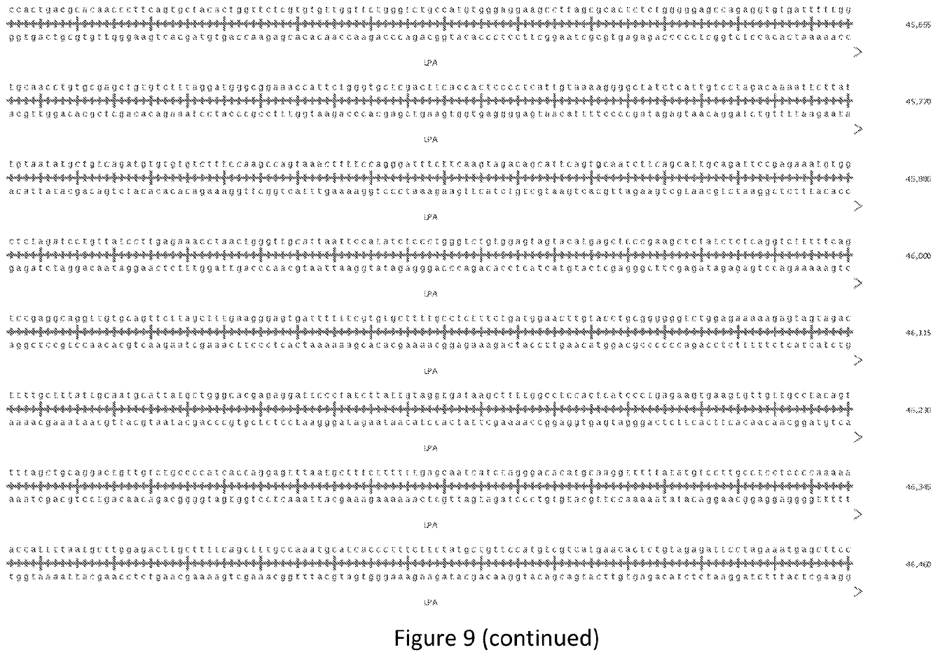

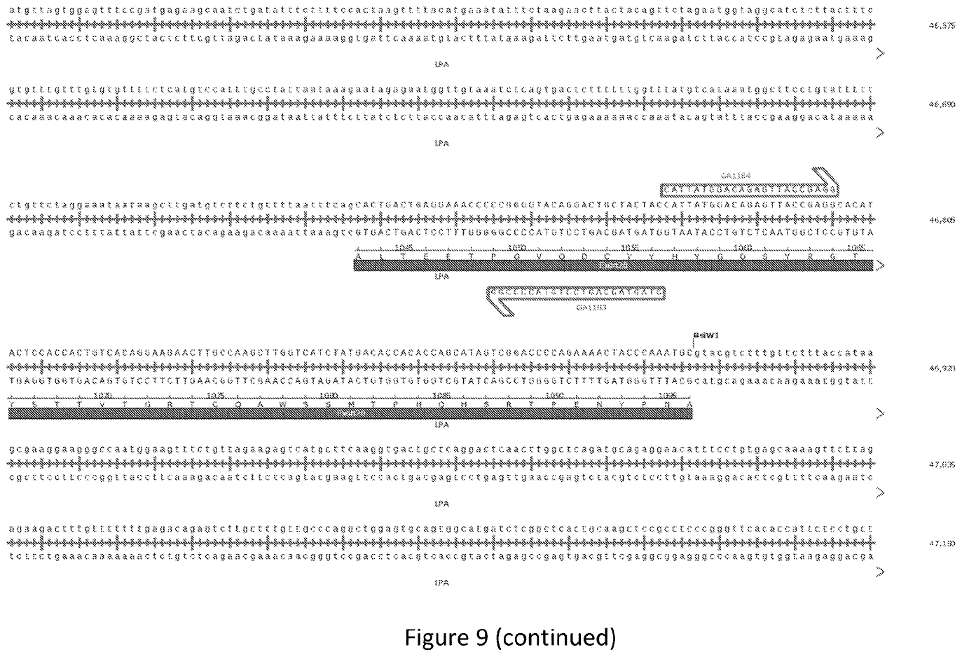

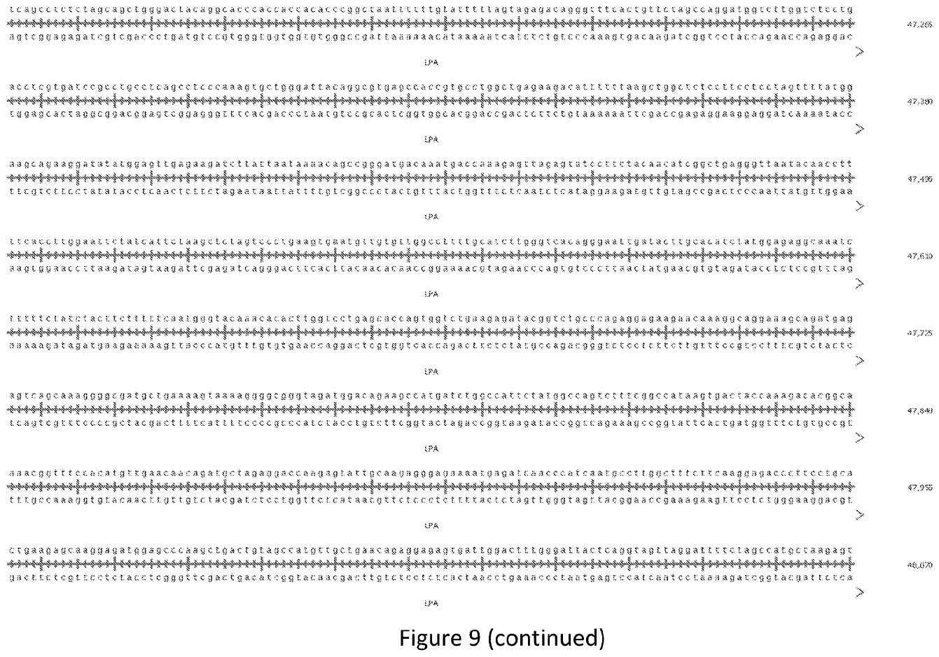

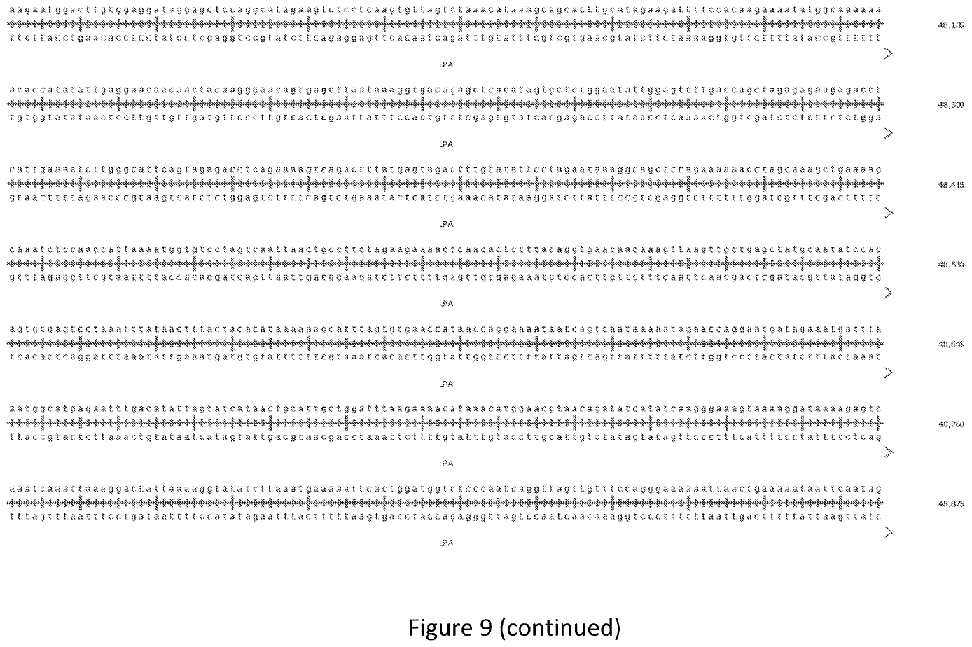

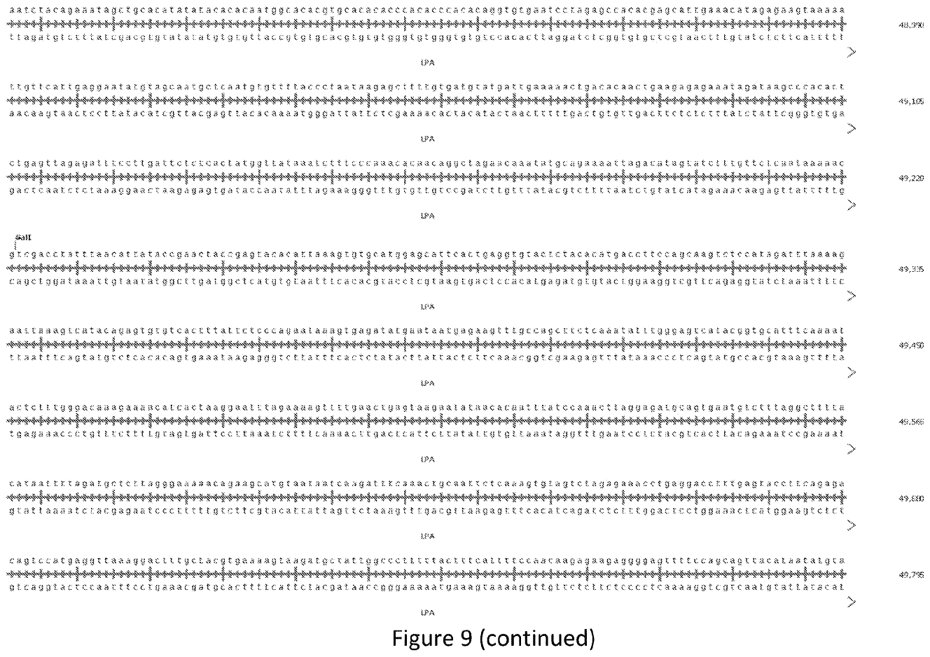

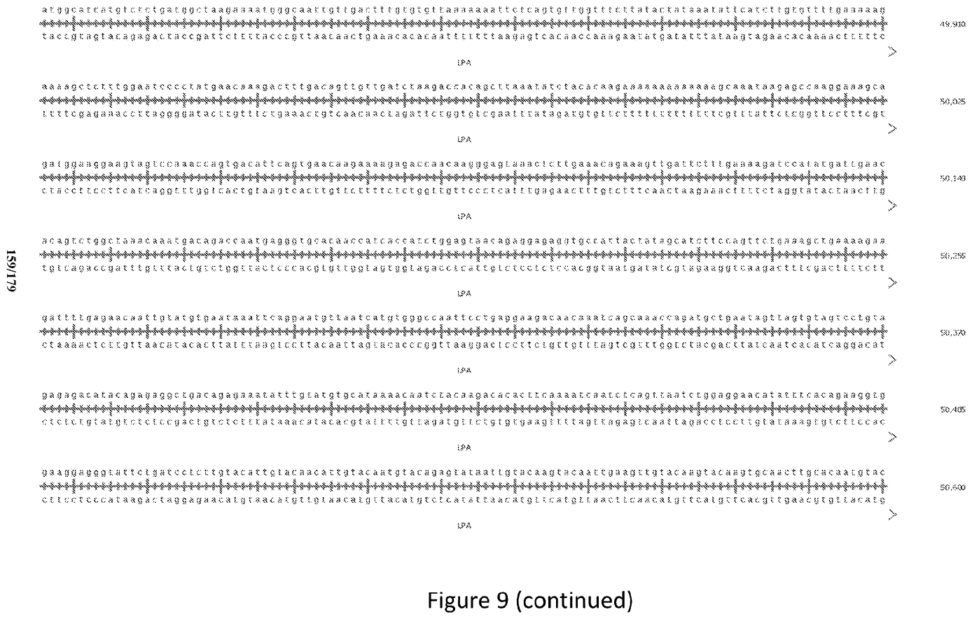

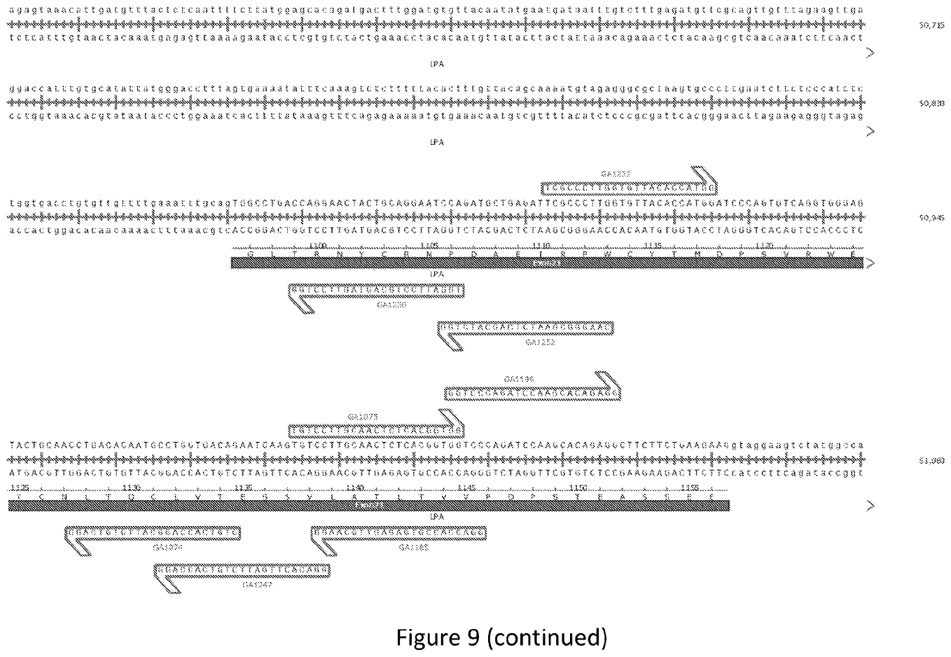

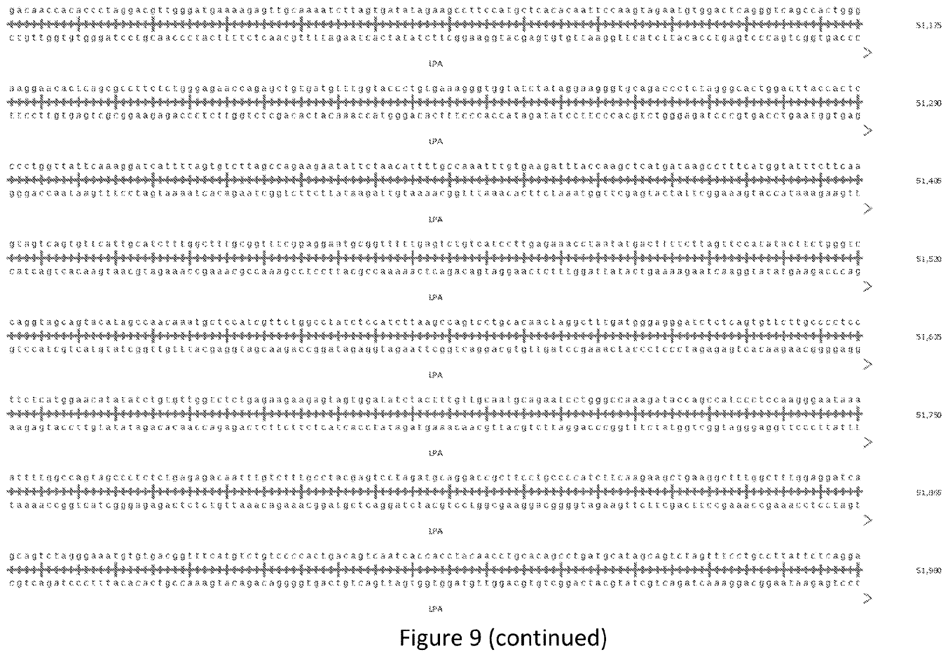

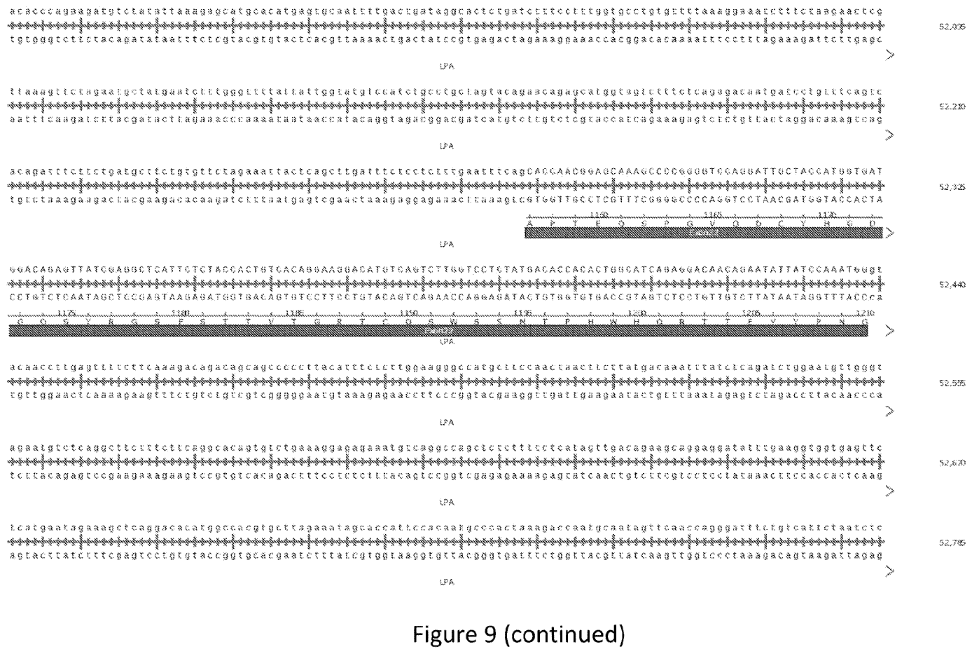

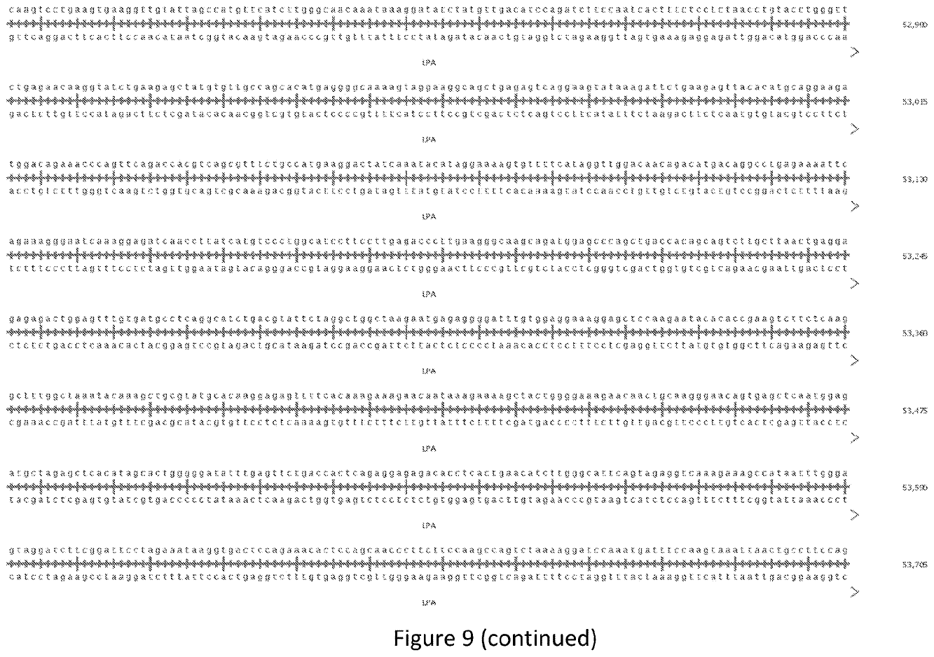

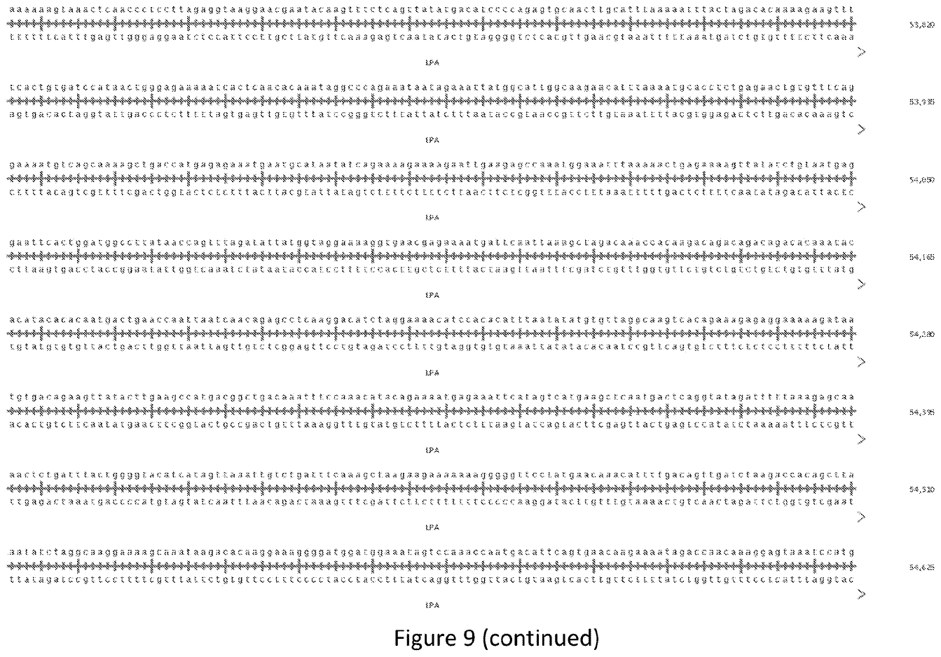

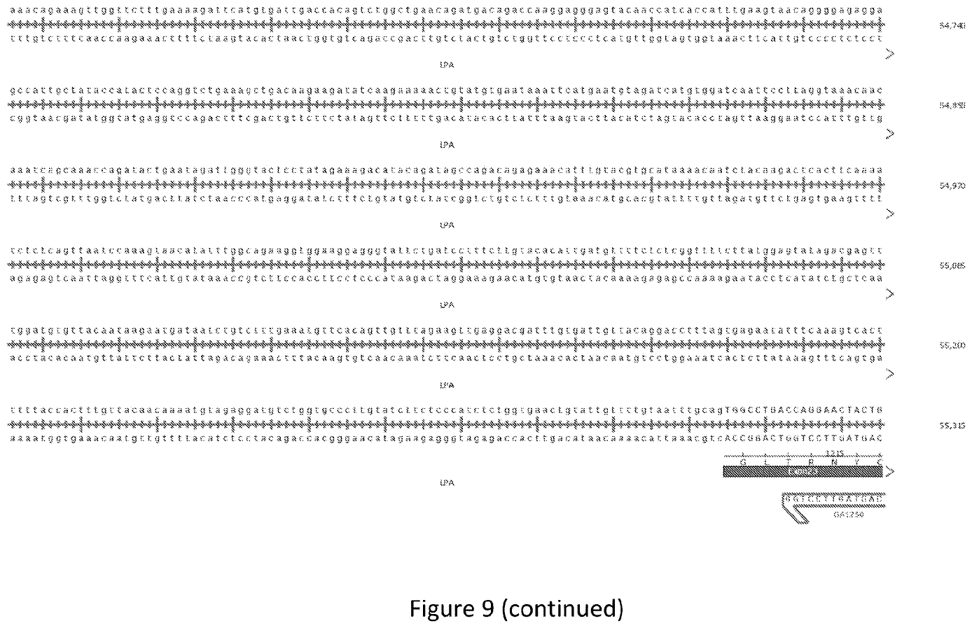

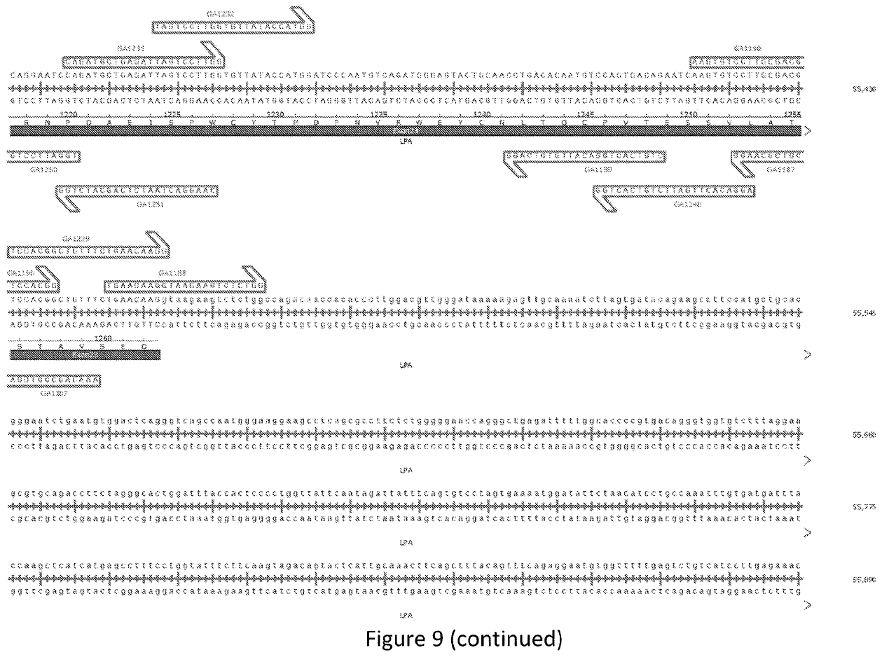

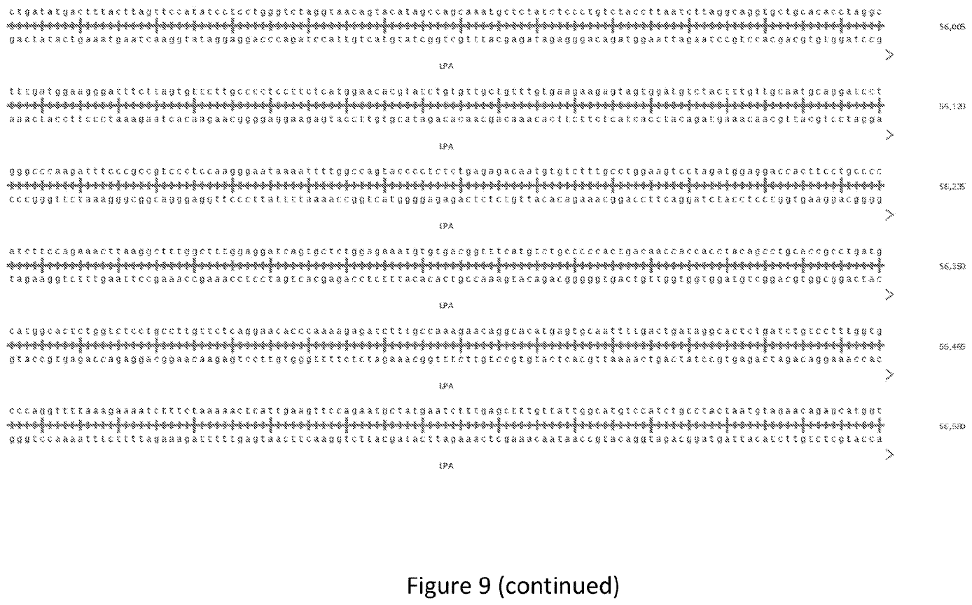

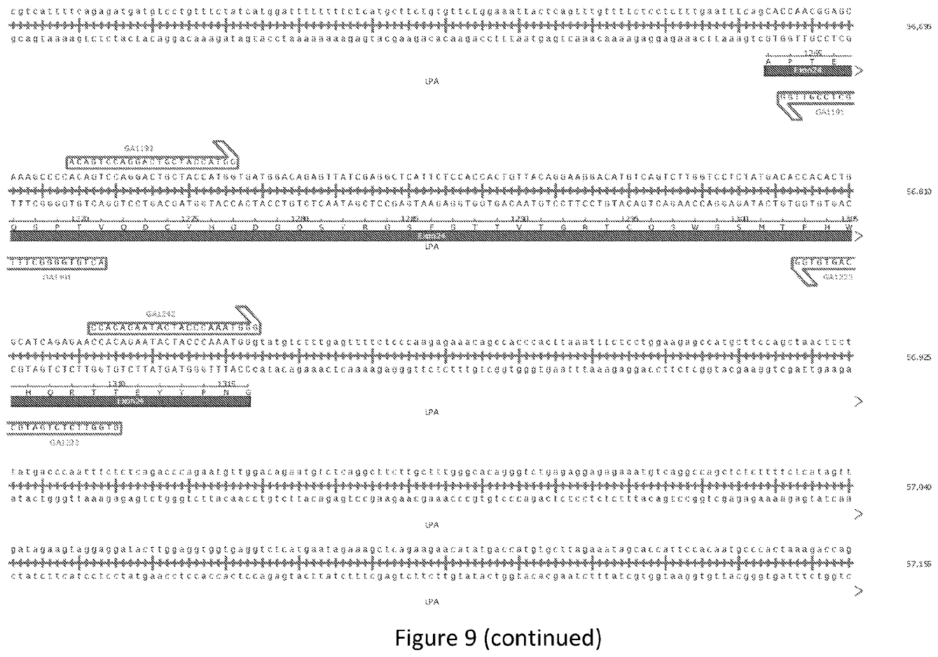

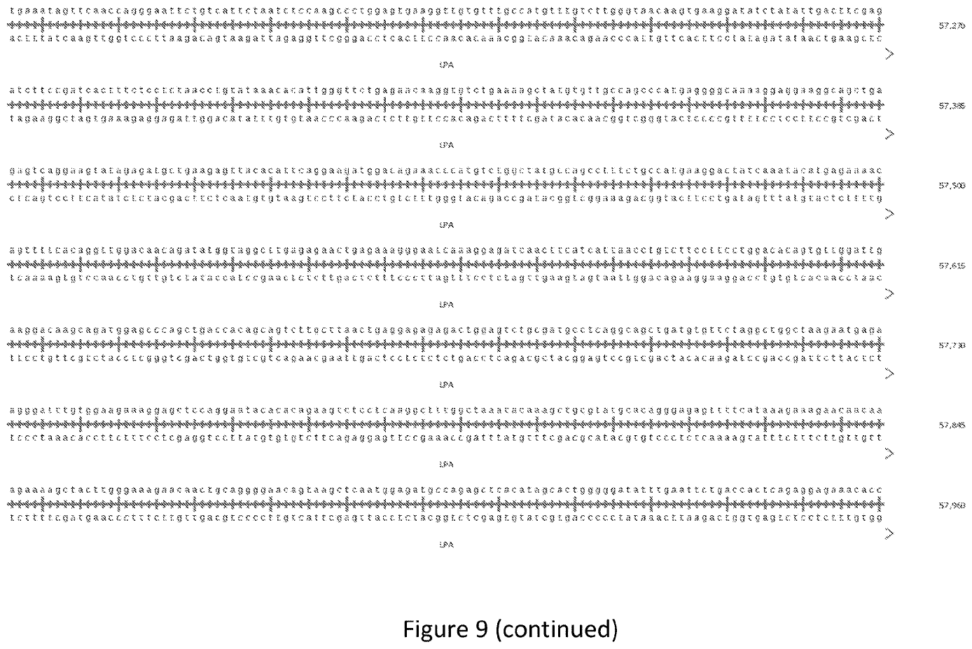

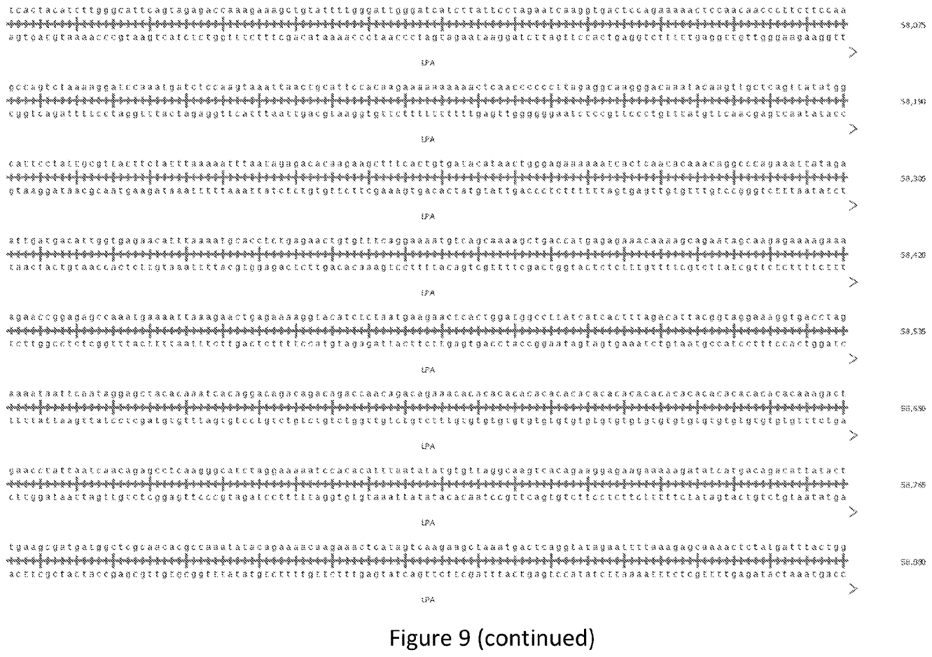

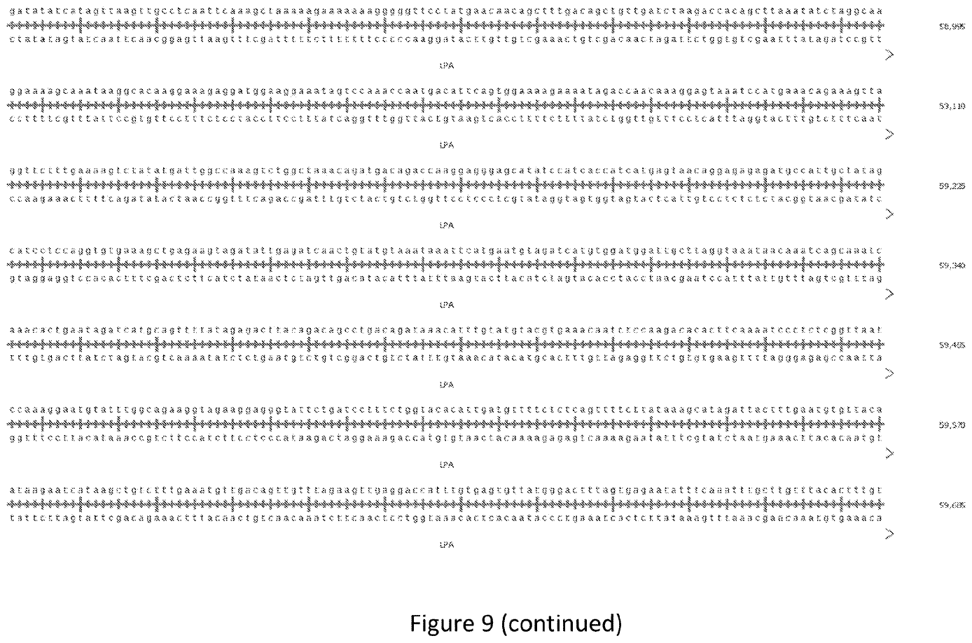

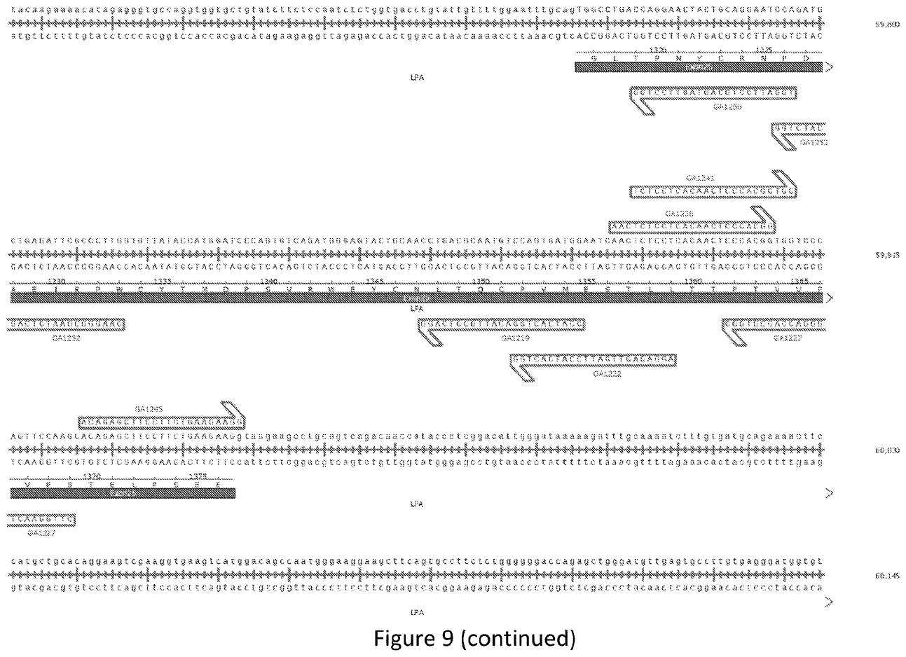

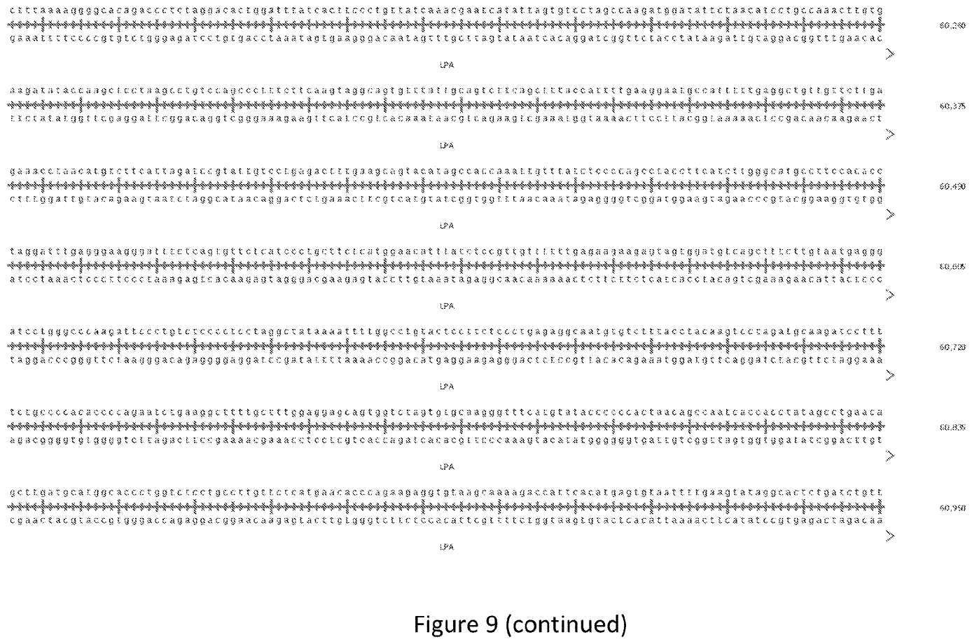

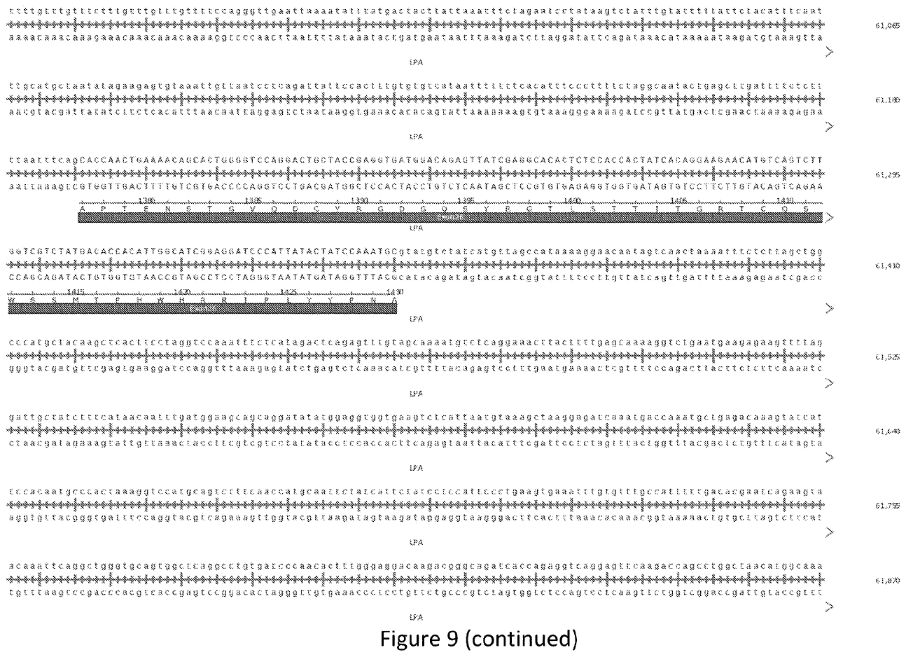

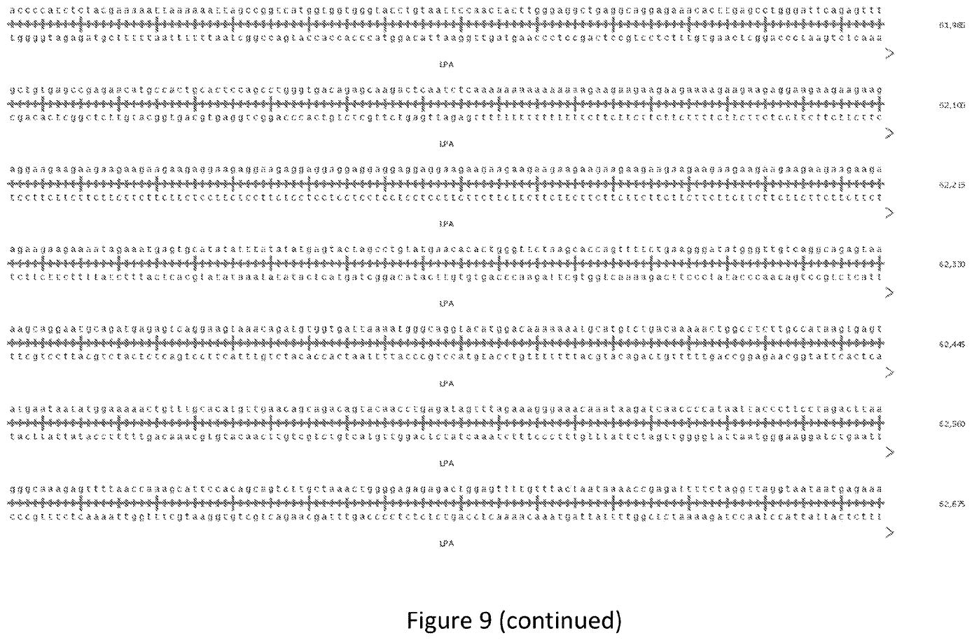

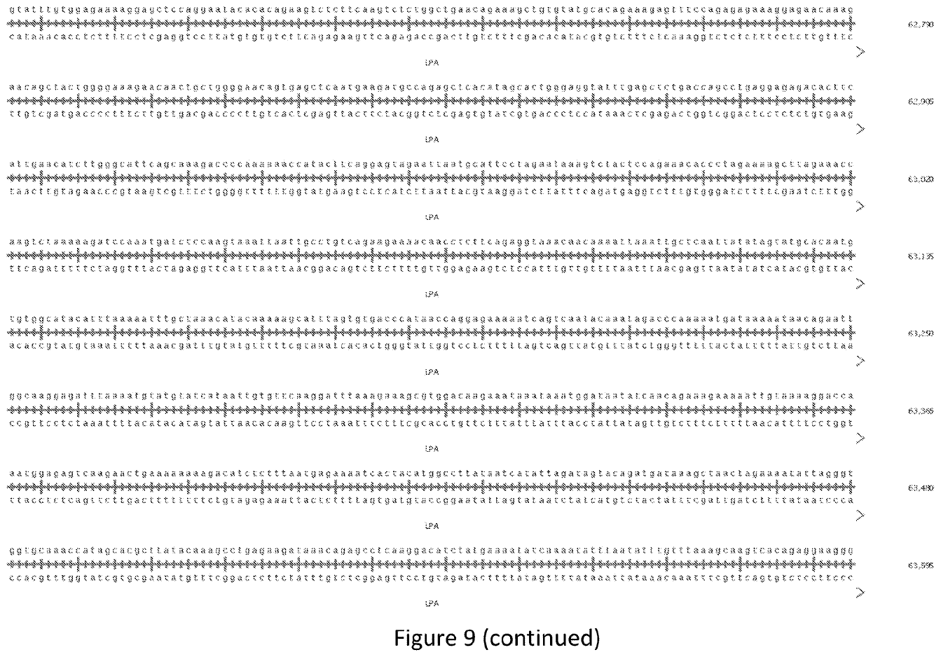

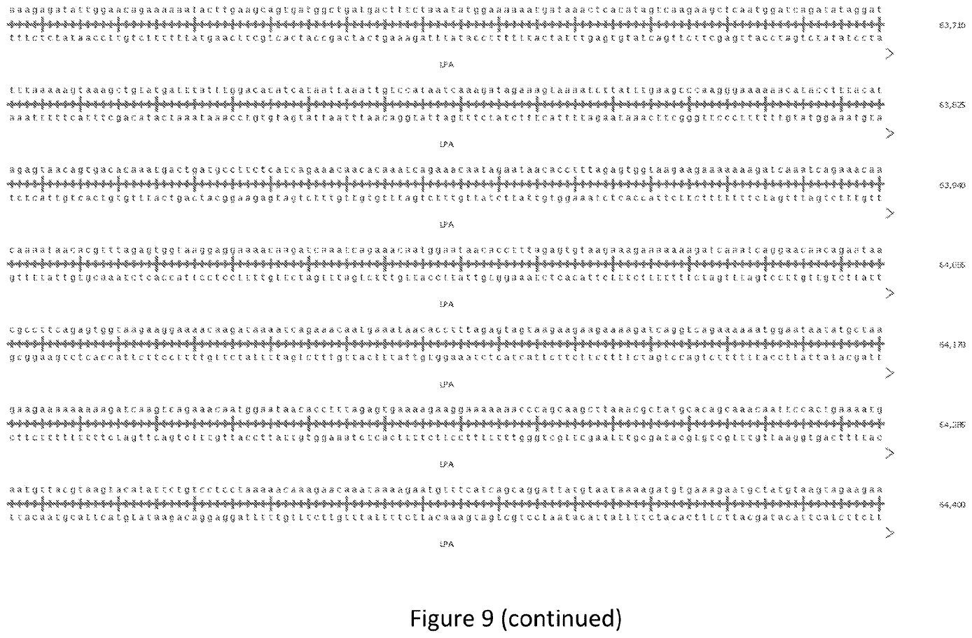

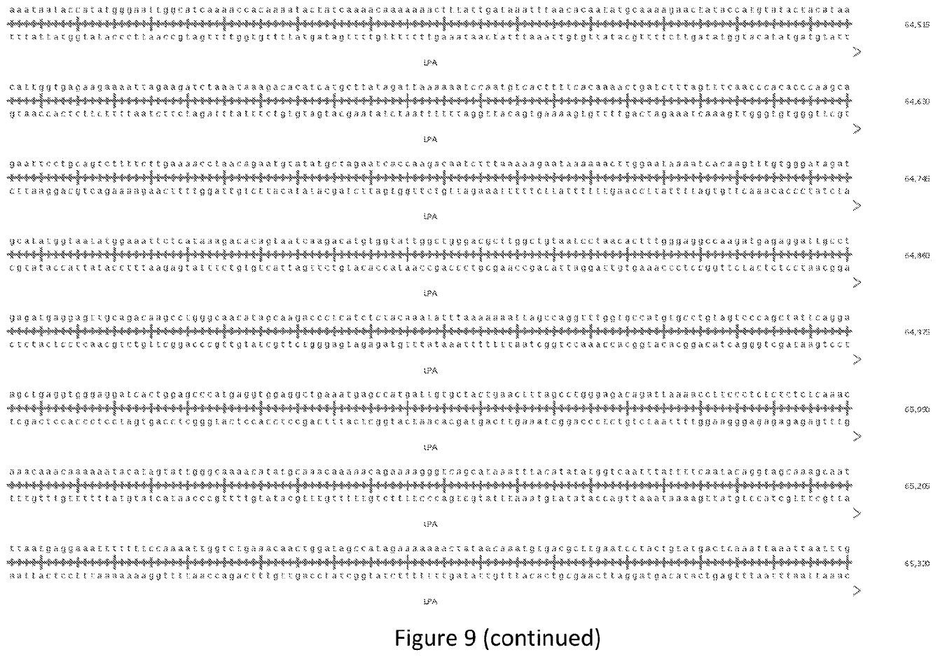

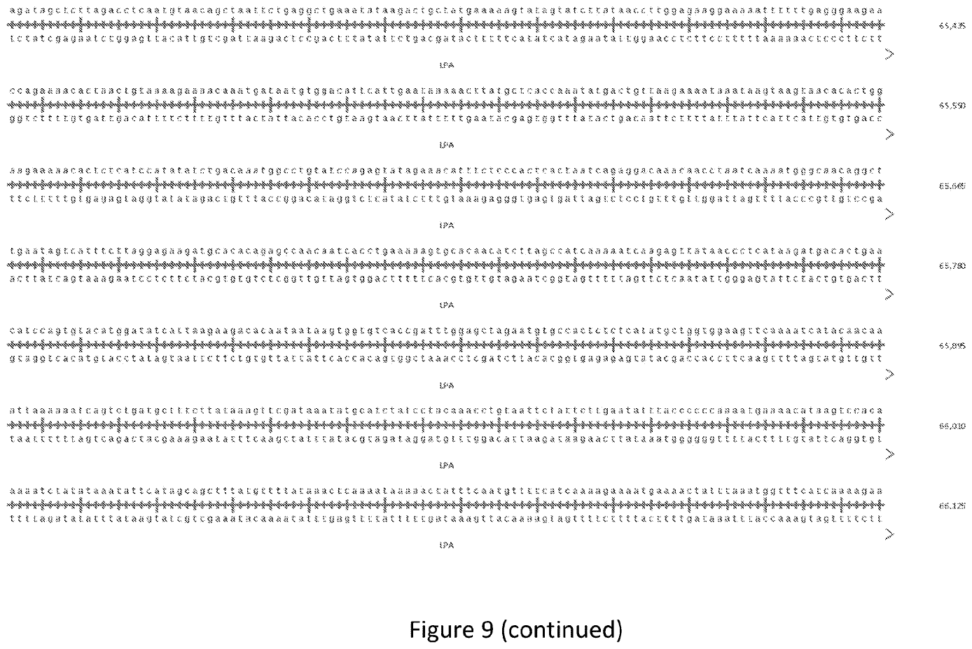

From among the potential guide RNAs having spacer sequences corresponding to the protospacer sequences in Table 2, 58 guide RNAs identified in Table 5 (provided above in the Detailed Description) were manufactured and used for further cellular analysis. The spacer sequences of some of the gRNAs and the location of hybridization to the LPA gene for some of the guide oligonucleotides are shown in FIG. 9.

### Cell Transfection

For primary human hepatocytes: collagen-I coated 96-well plates were seeded with primary human hepatocytes (BioIVT, lot: OQA) at a density of 5×10⁴ cells/well. Cells were plated into 100 μL of hepatocyte culture media and placed in an incubator for 4 hours, until they had attached and formed a monolayer.

For HuH-7 cells: 96-well plates were seeded with HuH-7 cells (Sekisui Xeno Tech) at a density of 2×10⁴ cells/well. Cells were plated into 100 μL of DMEM+10% FBS+1% penicillin/streptomycin and placed in an incubator for 24 hours, until they had attached and formed a monolayer.

TABLE 8
SpCas9-D10A and guide RNA experimental conditions

| mRNA/gRNA used | RNA amount (2500 ng/ml dose) | |
|---|---|---|
| guide RNA 1 (gRNA) | 62.5 | ng |
| guide RNA 2 (gRNA) | 62.5 | ng |
| SpCas9-D10A mRNA | 125 | ng |

For one experimental set, transfection reactions were prepared by combining RNA for a total dose of 2500 ng/ml, as listed in Table 8, and then adding Lipofectamine MessengerMax diluted with OPTI-MEM and incubating for 15 minutes at room temperature. For a second experimental set, transfection reagents were diluted 1:8, for a final dose of 312.5 ng/ml.

The hepatocyte culture medium was replaced with 90 μL of fresh hepatocyte culture medium, and 10 μL of each prepared transfection reaction was added to wells of cells in the 96-well plate for a total volume of 100 μL. Cells were incubated for an additional 48 hours, after which they were lysed and DNA extracted using Quick Extract reagent (Lucigen) as per the manufacturer's instructions; ~10 ng of DNA was used for targeted amplicon sequencing.

### Next-Generation Sequencing and Analysis

Genomic DNA was extracted from collected cells and subjected to next-generation sequencing with the Illumina MiSeq platform, generating 151-bp paired-end reads.

To evaluate the efficiency of SpCas9-D10A and guide RNAs to cause indel variants, computational analysis was used to determine what fraction of the reads mapping to the specific targeted LPA genomic region contained the desired edits. The % of reads containing desired edits was then used as a metric of dual nicking guide editing efficiency for each combination of SpCas9-D10A and guide RNAs. The quantifications of editing were plotted as editing percentages.

FIG. 2 shows edits for tested combinations of guide RNAs achieved with a higher dose (2500 ng/ml of RNA; closed circles) and a lower dose (312.5 ng/ml of RNA; open circles). More than half (31) of the tested guide pairs exceeded 50% editing at the higher (2500 ng/ml) dose; fifteen of the tested guide pairs exceeded 80% editing at the higher dose; and two of the tested guide pairs exceeded 90% editing at the higher dose.

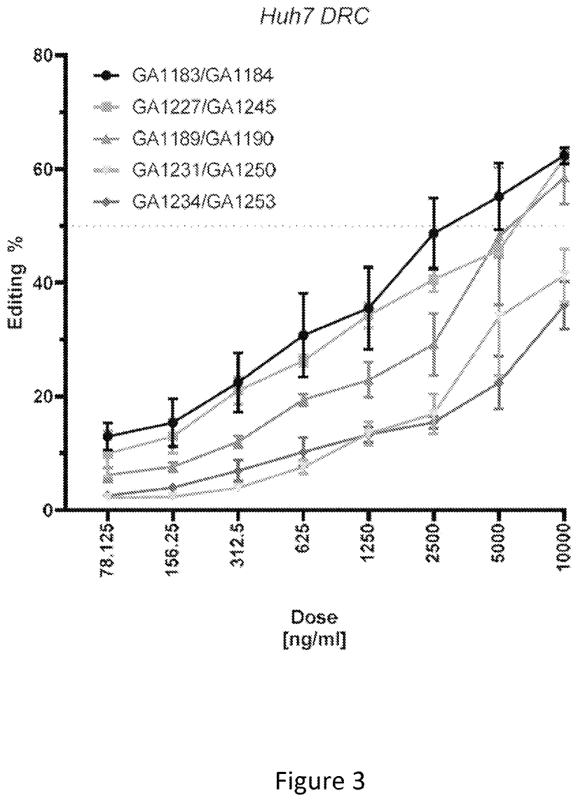

Some selected guide pairs were further tested. FIG. 3 shows edits for tested combinations of guide RNAs achieved with a highest dose (10,000 ng/ml of RNA) and 1:2 dilutions of dose down to the lowest dose (78.125 ng/ml of RNA). Guide 1:guide 2:mRNA weight ratios used were 1:1:2. Each tested combination yielded some level of editing. The highest level of editing observed with the highest RNA dose was 62% with guide pair GA1183/GA1184. Each of the 5 guide pairs demonstrated good potency, which was titratable in relation to the dose used. Thus, all guide RNAs introduced indel variants, and the edits were dose-dependent, validating the editing strategy.

### Example 3

### Evaluation of Efficiency of Nickase-Based Editing Using SpCas9-D10A and Guide/Template RNAs Via LNPs

With this example, several guide RNAs targeting the LPA gene were designed, paired based on proximity to one another, synthetically manufactured/prepared, and administered to cells via lipid nanoparticles (LNPs) to determine whether they were capable of editing and their respective editing efficiencies.

FIG. 4A schematically illustrates the locations at which spacer sequences of guide nucleic acids bind protospacer sequences in exon 20 of the LPA gene. The guide pairs tested in this example include GA1295/GA1296 (corresponding to LNP1); GA1297/GA1296 (corresponding to LNP2); and GA1298/GA1296 (corresponding to LNP3). The sequences of the guide oligonucleotides are provided in Table 4.

LNPs comprising a guide pair GA1295/GA1296 (corresponding to LNP 1), GA1297/GA1296 (corresponding to LNP 2), or GA1298/GA1296 (corresponding to LNP 3) and comprising mRNA encoding SpCas9-D10A (MS029) were prepared with equal weights of each gRNA per guide pair and with a 1:1 weight ratio of total gRNA (guide 1+guide 2) to mRNA.

The constituents of each of the LNPs were an ionizable amino lipid (iLipid), a neutral helper lipid, a PEG-Lipid, and a sterol lipid, with optional inclusion of a ligand-targeting lipid, such as GalNAc. Table 9 below describes LNP formulations, excluding the drug substances (i.e., gRNA and mRNA).

TABLE 9
LNP Components

| LNP | | | Mol% | | | | |
|---|---|---|---|---|---|---|---|
| Component | Lipid names | Lipid structure | Range | Mol% Examples | | | |
| Amino lipid (iLipid) | 3-((4,4-bis(octyloxy) butanoy1)oxy)-2-((((3- (diethylamino)propoxy) carbonyl)oxy) methyl)propyl (9Z,12Z)-octadeca- 9,12-dienoate* | 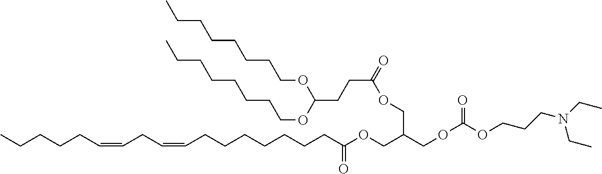 [SMILES: CCCCC/C=C\C/C=C\CCCCCCCC(=O)OCC(COC(=O)CCC(OCCCCCCCC)OCCCCCCCC)COC(=O)OCCCN(CC)CC] | 40-60 | 45 | 47 | 50 | 52.4 |
| Neutral helper lipid | 1,2-distearoyl-sn-glycero- 3-phosphocholine (DSPC) | 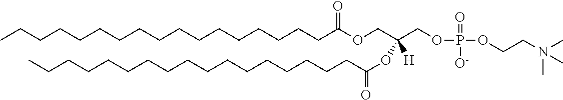 | 4-20 | 9 | 9 | 9, 10 | 9, 10 |
| PEG- lipid | 1,2-dimyristoyl- rac-glycero- 3-methoxypolyethylene glycol-2000 (PEG₂₀₀₀-DMG) | 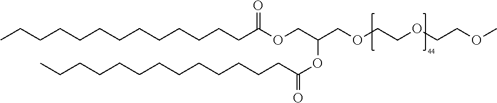 | 1.5-5 | 2 | 3 | 1.5, 3 | 2.5, 3 |
| Sterol lipid | Cholesterol | 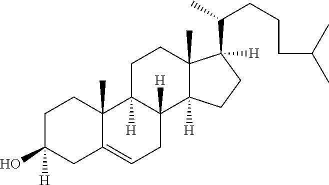 | 30-45 | Balance of Mol% to make 100% | Balance of Mol% to make 100% | Balance of Mol% to make 100% | Balance of Mo1% to make 100% |

*described in International Published Patent Application WO 2021/178725 A1

It should be understood that the lipids in Table 9 may be substituted for other suitable lipids in the listed class. In some embodiments, for example, the LNP comprises the amino lipid VL422 described in the International Published Patent Application WO 2022/060871 A1. For example, the amino lipid may be VL422 or a pharmaceutically acceptable salt or solvate thereof:

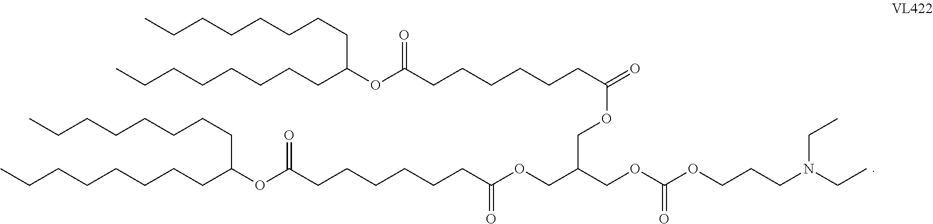 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]

It should be further understood that the example mol % of lipids in Table 9 thus may be adjusted and that the mol % included in Table 9 are targeted excipient percentages of the LNP, which is intended to represent the aggregate mol % of all the LNPs formulated in a given batch and that specific LNPs within a batch may have varying mol %. Thus, it is contemplated herein that the mol % of one or more or all of the LNP components set forth in Table 9 may be adjusted, for example, by +/−1 to 5%, +/−5 to 10%, or +/−10% to 20%. It is further contemplated herein that the mol % of one or more or all of the LNP components set forth in Table 9 with respect to a specific LNP, formulated in a given batch of LNPs formulated in accordance with desired target excipient percentages, may vary from the targeted mol %, for example, by +/−1 to 5%, +/−5 to 10%, +/−10 to 20%, or even greater than +/−20%. Further, it should be understood that additional LNP components, including non-lipid components, may be added to the LNP components set forth in Table 9. Thus, for example, GalNAc ligand-targeting lipids such as those disclosed in International Published Patent Application WO 2021/178725 A1 may be added and the cholesterol mol % component reduced to account for the additional GalNAc conjugated lipid. Thus, for example, if 0.05% GalNAc lipid is added to the formulation, the mol % of cholesterol in the formulation would be reduced by 0.05%. Examples of GalNAc lipids used in LNP formulations included:

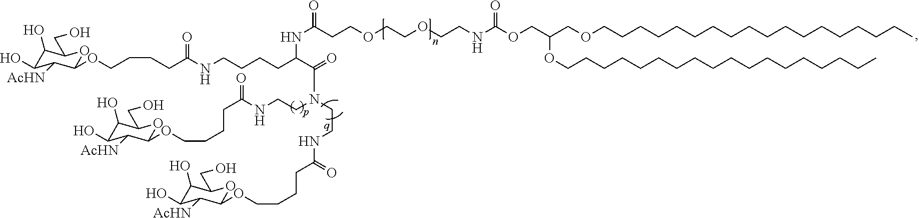

wherein each of the p and q is independently an integer from 1 to 5, and n is an integer from 33 to 39; and

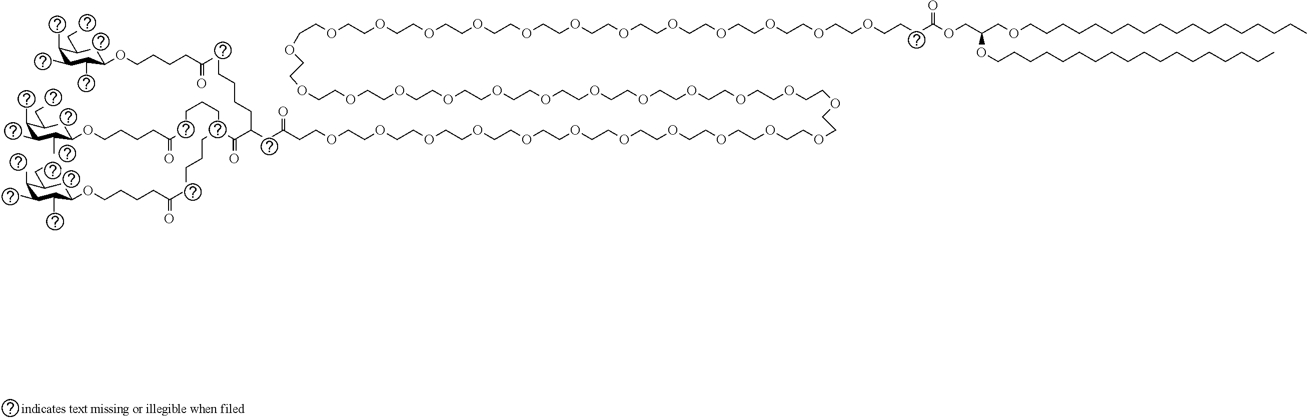

The average LNP particle size may range from 40-90 nm, 50-80 nm, or 55-75 nm, +/−15 nm.

Primary human hepatocyte (PHH) cells were incubated with LNPs (LNP 1, LNP 2, LNP 3) at various concentrations ranging from 0 to 40,000 ng/mL of total RNA (guides and mRNA). Cells were harvested, and editing efficiency were evaluated as described in Example 2.

Results are shown in FIG. 4B, which illustrates that greater than 95% editing efficiency was observed for all three pairs at a dose of 10,000 ng (total RNA)/ml. EC₅₀ and EC₉₀ values were similar for all three guide pairs.

This Example illustrates that high levels of editing efficiency can be achieved via LNP delivery of the gene editing system.

### Example 4

### Reduction in Secreted LP(a) Protein in HuH-7 Cells by Gene Editing System

Lentiviruses containing an expression cassette comprising an LPA open reading frame (ORF) followed by an internal ribosome entry site (IRES) and puromycin N-acetyltransferase (puro), driven by a cytomegalovirus (CMV) promoter, were created. HuH-7 cells were transfected with the lentiviruses and selected for integration of the expression cassette with puromycin.

LNPs (LNP1) as described in Example 3 were administered to puromycin-resistant cells in which the expression cassette was integrated into the genome (the guide pair was GA1295/1296) at various concentrations ranging from 0 to 5,000 ng/ml (total RNA), and levels of apo(a) secreted from the cells were evaluated. Apo(a) protein concentration was assessed using a validated Lp(a) ELISA kit from Mercodia.

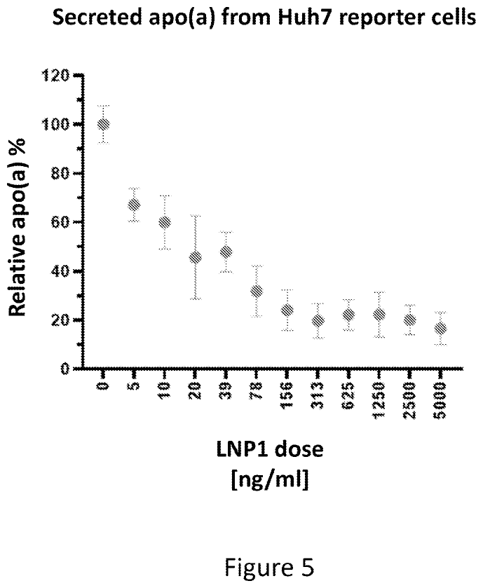

As shown in FIG. 5, secreted apo(a) was reduced in a dose-dependent manner, illustrating that gene editing as described herein may be used to lower apo(a) levels.

### Example 5

### Evaluation of Off-Target Editing

Off-target analysis was performed and indicated a very favorable off-target profile (data not shown).

### Example 6

### Gene Editing in Transgenic Mice Expressing Human LPA Gene

Transgenic mice expressing the human LPA gene (J:144538 Frazer K A, et al., The apolipoprotein(a) gene is regulated by sex hormones and acute-phase inducers in YAC transgenic mice. Nat Genet. 1995 April; 9(4):424-31) were obtained from The Jackson Laboratory. Groups of transgenic mice (two females and two males per group) were split as follows: control vehicle (LNP), 0.05 mg/kg total dose RNA, 0.1 mg/kg total dose RNA, 0.5 mg/kg total dose RNA, 2 mg/kg total dose RNA, 4 mg/kg total dose RNA, and 10 mg/kg total dose RNA.

The LNPs were prepared as described in Example 3 and included guide RNA pair GA1295/GA1296 and mRNA encoding SpCas9-D10A (MS029) in a ratio of 1:1:2 (or 1:1 based on total guide RNA to mRNA). The sequences of the guide oligonucleotides are provided in Table 4.

LNP formulations were administered via retro-orbital injection.

Serum apo(a) protein concentrations were determined seven days prior to injecting the mice with LNPs, seven days following injection, and fourteen days following injection. Serum apo(a) protein concentrations were evaluated using a validated Lp(a) ELISA kit from Mercodia.

At day 14, livers were harvested from the mice, and DNA extraction performed as described above in Example 2.

The average serum apo(a) protein concentration in female mice was about 50 ng/dL seven days prior to injection with the LNPs. The average concentration in male mice was about 1.25 ng/dL seven days prior to injection with the LNPs. Accordingly, the male apo(a) serum concentrations were about 40-fold less than the female serum concentrations.

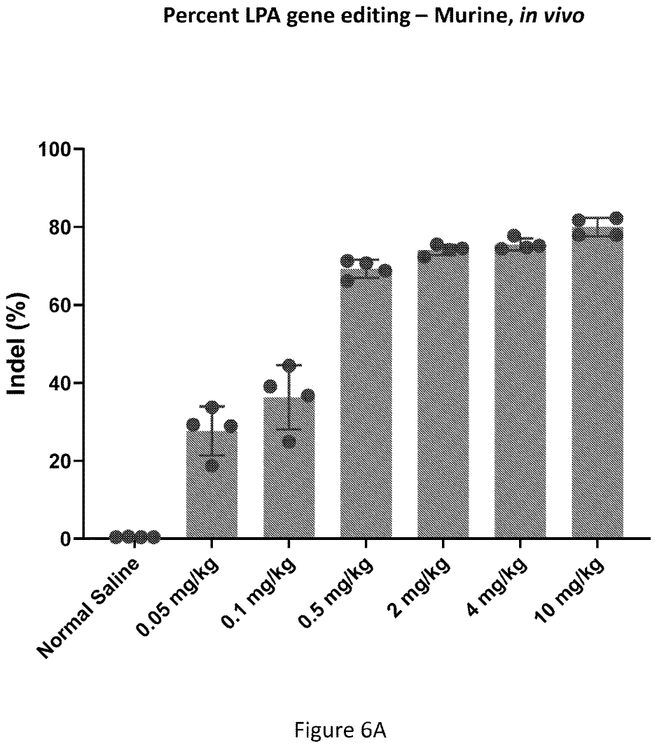

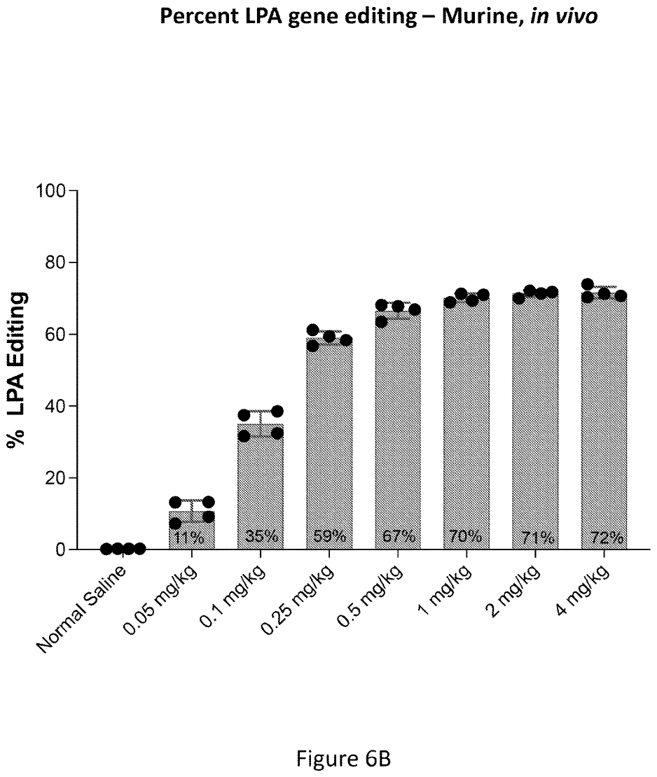

As shown in FIG. 6A, the editing efficiency of the LPA gene occurred in a dose-dependent manner. The EC₅₀ was determined to be 0.18 mg/kg, and the EC₉₀ was determined to be 0.67 mg/kg. Greater than 70% editing efficiency was achieved at the 2.0 mg/kg total RNA dose. The experiment was repeated with slightly different doses and with LNPs having different average particle size (67 nm versus 77 nm for FIG. 6A). The results are presented in FIG. 6B, which illustrates similar results to those illustrated in FIG. 6A.

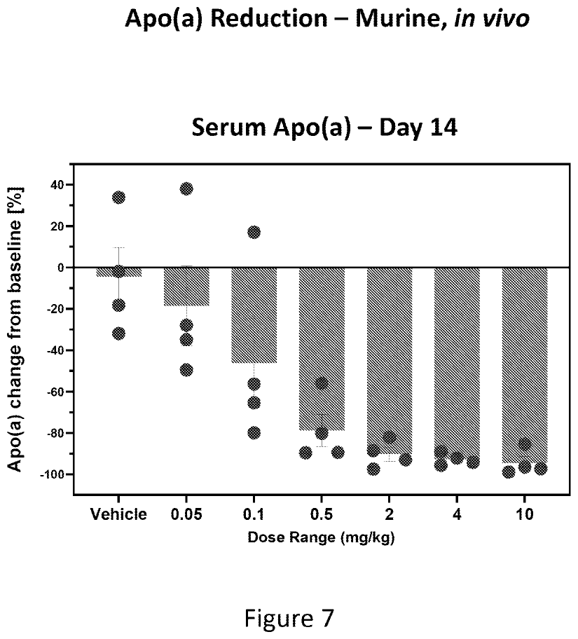

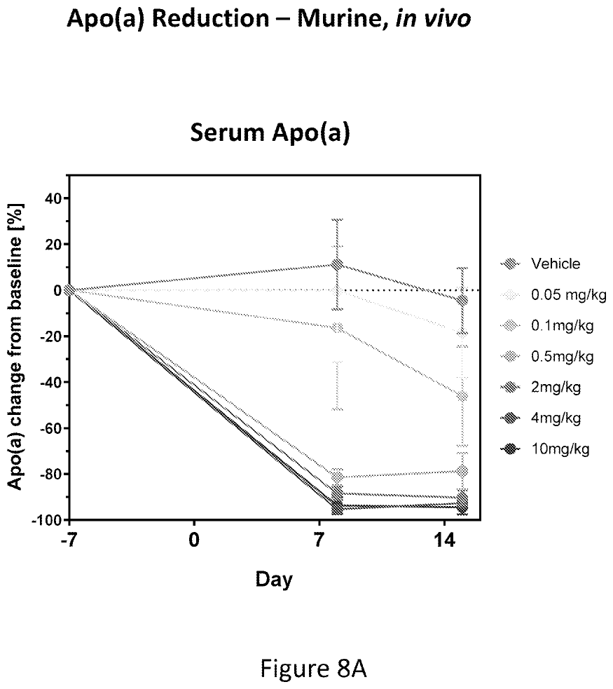

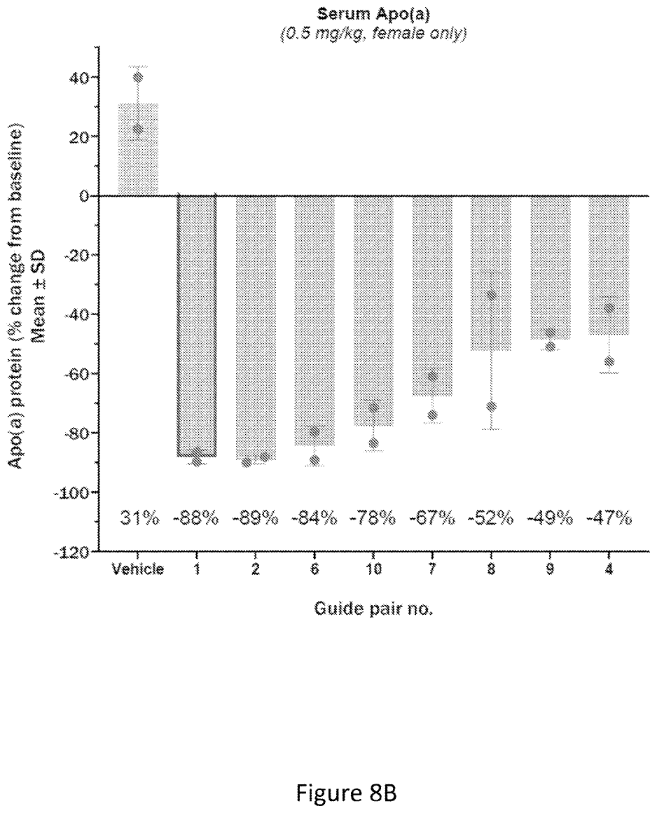

As shown in FIGS. 7 and 8A, delivery of the gene editing system knocked down serum apo(a) levels in the mice in a dose-dependent manner. Serum apo(a) was reduced by about 90% at the 2.0 mg/kg total RNA dose.

These results indicate that in vivo delivery of a gene editing system as described herein can introduce indels in the LPA gene, which results in reduction of serum levels of the apo(a) protein.

Gene editing systems comprising additional guide pairs were evaluated for their ability to reduce serum apo(a) concentrations in female transgenic mice when administered in LNPs at a concentration of 0.5 mg total RNA/kg. The experiments were generally performed as indicated above. Plasma apo(a) concentrations were determined at baseline (−7 days) and 14 days following retro-orbital injection of the gene editing systems. The results are presented in FIG. 8B.

The guide pair number shown in FIG. 8B corresponds to the guide pairs listed in Table 10. The sequences of the gRNA are listed in Table 4.

TABLE 10
Guide pair designators for guide pairs numbers shown in FIG. 8B

| Guide pair no. | Guide 1 | Guide 2 |
|---|---|---|
| 1 | GA1295 | GA1296 |
| 2 | GA1297 | GA1296 |
| 4 | GA1301 | GA1302 |
| 6 | GA1303 | GA1304 |
| 7 | GA1442 | GA1448 |
| 8 | GA1443 | GA1449 |
| 9 | GA1440 | GA1446 |
| 10 | GA1438 | GA1444 |

Guide pair GA1303/GA1304 is interesting because the guide pair targets Exon 2 of the LPA gene and exhibits high editing efficiency.

The subject matter described herein and in the accompanying figures is done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

### XII. OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment, any portion of the embodiment, or in combination with any other embodiments or any portion thereof.

As is set forth herein, it will be appreciated that the disclosure comprises specific embodiments and examples of base editing systems to effect a nucleobase alteration in a gene and methods of using same for treatment of disease including compositions that comprise such base editing systems, designs and modifications thereto; and specific examples and embodiments describing the synthesis, manufacture, use, and efficacy of the foregoing individually and in combination including as pharmaceutical compositions for treating disease and for in vivo and in vitro delivery of active agents to mammalian cells under described conditions.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:

Following is an example of enumerated clauses:
1. A pharmaceutical composition for in vivo editing of an LPA gene, in a mammalian subject, comprising:
   (i) an engineered, non-naturally occurring gene editing system, which comprises:
      (a) one or more polynucleotides (mRNAs) encoding one or more CRISPR Cas nickases;
      (b) a first guide oligonucleotide (gRNA) comprising a first spacer sequence that is complementary to a first strand of the LPA gene at a first target sequence and a first scaffold region that serves as a binding scaffold for at least one of the one or more Cas nickases; and
      (c) a second guide oligonucleotide (gRNA) comprising a second spacer sequence that is complementary to a second strand of the LPA gene at a second target sequence and a second scaffold region that serves as a binding scaffold for at least one of the one or more Cas nickases, and
   (ii) a delivery system that is engineered to deliver the one or more mRNAs, the first gRNA, and/or the second gRNA individually or collectively to the liver,
   wherein the first gRNA and the at least one of the one or more Cas nickases are engineered to cause the at least one of the one or more Cas nickases to nick one of the first or second strands of the LPA gene at a first location of chromosome 6 from position 160,664,275 to 160,531,482, and
   wherein the second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.
2. The pharmaceutical composition of clause 1, wherein the delivery system comprises lipid nanoparticles (LNPs), wherein the LNPs comprise:
   (a) one or more ionizable lipids,
   (b) cholesterol,
   (c) one or more PEG-lipids,
   (d) a phospholipid; and
   (e) optionally including a targeting moiety, such as a GalNAc lipid.
3. The pharmaceutical composition of clause 2, wherein the LNPs are formulated to comprise:
(a) 40 to 60 molar percent of the one or more ionizable lipids, 28 to 48 molar percent of the cholesterol, 5 to 13 molar percent of the phospholipid, and 2 to 5 molar percent of the PEG-lipid,
(b) 50+/−10% molar percent of the one or more ionizable lipids, 38+/−10% molar percent of the cholesterol, 9+/−10% molar percent of the phospholipid, and 3+/−10% molar percent of the PEG-lipid,
(c) 40 to 60 molar percent of the one or more ionizable lipids, 27.95 to 47.95 molar percent of the cholesterol, 5 to 13 molar percent of the phospholipid, 2 to 5 molar percent of the PEG-lipid, and 0.02 to 0.09 molar percent of the GalNAc-lipid; or
(d) 50+/−10% molar percent of the one or more ionizable lipids, 37.95+/−10% molar percent of the cholesterol, 9+/−10% molar percent of the phospholipid, 3+/−10% molar percent of the PEG-lipid, and 0.05+/−10% molar percent of the GalNAc-lipid.
4. The pharmaceutical composition of any one of clauses 1 to 3, wherein the gene editing system is engineered to inactivate the LPA gene.
5. The pharmaceutical composition of clause 4, wherein inactivating the LPA gene results in a lack of production of apo(a) protein or the production of non-functional apo(a) protein, resulting in a reduced blood Lp(a) concentration.
6. The pharmaceutical composition of any one of clause 1 to 5, wherein at least one of the one or more Cas nickases, when in operative interaction with the first or second guide oligonucleotide, is engineered to nick the opposite strand of the LPA gene to which the operative guide oligonucleotide (gRNA) is hybridized.
7. The pharmaceutical composition of clause 6, wherein at least one of the one or more polynucleotides (mRNAs) encoding the one or more Cas nickases encodes a Streptococcus pyogenes Cas9 nickase bearing a H840A mutation.
8. The pharmaceutical composition of any one of clauses 1 to 5, wherein at least one of the one or more Cas nickases, when in operative interaction with the first or second guide oligonucleotide (gRNA), is engineered to nick the same strand of the LPA gene to which the operative guide is hybridized.
9. The pharmaceutical composition of clause 8, wherein at least one of the one or more polynucleotides (mRNAs) encoding the one or more Cas nickases encodes a Streptococcus pyogenes Cas9 nickase bearing a D10A mutation.
10. The pharmaceutical composition of any one of clauses 1 to 9, wherein the sequences of the first and second scaffold region are the same.
11. The pharmaceutical composition of any one of clauses 1 to 10, wherein the first spacer sequence of the first guide oligonucleotide (gRNA) comprises about 15 to about 26 nucleotides and has a sequence that is identical to or substantially identical to a targeted first protospacer sequence adjacent to a protospacer-adjacent motif (PAM) sequence on the first strand of the LPA gene.
12. The pharmaceutical composition of any one of clauses 1 to 11, wherein the second spacer sequence of the second guide oligonucleotide (gRNA) comprises about 15 to about 26 nucleotides and has a sequence that is identical to or substantially identical to a second targeted protospacer sequence adjacent to a protospacer-adjacent motif (PAM) sequence on the second strand of the LPA gene.
13. The pharmaceutical composition of any one of clauses 1 to 12, wherein the gene editing system is engineered to edit the LPA gene to effect an indel or non-synonymous variant.
14. The pharmaceutical composition of any one of clauses 1 to 13, wherein the first spacer sequence of the guide oligonucleotide (gRNA) has sequence that is identical to or substantially identical to a first protospacer sequence adjacent to a first protospacer-adjacent motif (PAM) sequence on the first strand of the LPA gene, and the second spacer sequence of the second guide oligonucleotide (gRNA) has sequence that is identical to or substantially identical to a second protospacer sequence adjacent to a second protospacer-adjacent motif (PAM) sequence on the second strand of the LPA gene, and
   wherein the one or more Cas nickases, in operation with the first and second guide oligonucleotides, is engineered to nick opposing strands of the LPA gene between the first and second protospacer-adjacent motifs (PAMs) in a “PAM-out” configuration.
15. The pharmaceutical composition of any one of clauses 1 to 13, wherein the first spacer sequence of the first guide oligonucleotide (gRNA) has a sequence that is identical to or substantially identical to a first protospacer sequence adjacent to a first protospacer-adjacent motif (PAM) sequence on the first strand of the LPA gene, and the second spacer sequence of the second guide oligonucleotide (gRNA) has a sequence that is identical to or substantially identical to a second protospacer sequence adjacent to a second protospacer-adjacent motif (PAM) sequence on the second strand of the LPA gene, and
   wherein the one or more Cas nickases, in operation with the first and second guide oligonucleotides, is engineered to nick opposing strands of the LPA gene outside the first and second protospacer-adjacent motifs (PAMs) in a “PAM-in” configuration.
16. The pharmaceutical composition of any one of clauses 1 to 15, wherein the one or more mRNAs encode a single CRISPR Cas nickase.
17. The pharmaceutical composition of any one of clauses 1 to 16, wherein the one or more Cas nickases in operation with the first and second guide oligonucleotides (gRNAs) are engineered to be directed to first and second target sequences opposite of respective first and second protospacer sequences within or in proximity to exon 25 of the LPA gene, which spans from chromosome 6, position 160,586,630, to chromosome 6, position 160,586,449, and to nick opposing strands of the LPA gene to effect indel variants and/or non-synonymous variants in the LPA gene.
18. The pharmaceutical composition of any one of clauses 1 to 16, wherein the one or more Cas nickases in operation with the first and second guide oligonucleotides (gRNAs) are engineered to be directed to first and second target sequences opposite of respective first and second protospacer sequences within or in proximity to exon 19 of the LPA gene, which spans from chromosome 6, position 160,601,098, to chromosome 6, position 160,600,917, and to nick opposing strands of the LPA gene to effect indel variants and/or non-synonymous variants in the LPA gene.
19. The pharmaceutical composition of any one of clauses 1 to 16, wherein the one or more Cas nickase in operation with the first and second guide oligonucleotides (gRNAs) are engineered to be directed to first and second target sequences opposite of respective first and second protospacer sequences within or in proximity to exon 31 of the LPA gene, which spans from chromosome 6, position 160,548,659, to chromosome 6, position 160,548,478, and to nick opposing strands of the LPA gene to effect indel variants and/or non-synonymous variants in the LPA gene.
20. The pharmaceutical composition of any one of clauses 1 to 19,
   wherein the first guide oligonucleotide comprises a first spacer having a sequence identical to, or at least about 75%, at least about 80%, at least about 85%, at least about 90%, or at least about 95% identity to, a protospacer listed in Table 2 or Table 5; and
   the second guide oligonucleotide comprises a second spacer having a sequence identical to, or at least about 75%, at least about 80%, at least about 85%, at least about 90%, or at least about 95% identity to, a protospacer listed in Table 2 or Table 5,
   wherein the first spacer corresponds to a protospacer on one strand of the LPA gene and the second spacer corresponds to a protospacer on the other strand of the LPA gene.
21. The pharmaceutical composition of any one of clauses 1 to 19, wherein
   the first guide oligonucleotide comprises a first spacer having a sequence identical to, or at least about 75%, at least about 80%, at least about 85%, at least about 90%, or at least about 95% identity to, a guide 1 protospacer listed in Table 2 or Table 5; and
   the second guide oligonucleotide comprises a second spacer having a sequence identical to, or at least about 75%, at least about 80%, at least about 85%, at least about 90%, or at least about 95% identity to, a guide 2 protospacer listed in Table 2 or Table 5,
   wherein the guide 1 protospacer and the guide 2 protospacer are in a same row of Table 2 or Table 5.
22. The pharmaceutical composition of any one of clauses 1 to 19, wherein
   the first guide oligonucleotide comprises a first spacer having a sequence identical or substantially identical to a guide 1 protospacer sequence listed in Table 2 or Table 5, or a 3′ portion thereof, with 0, 1, 2, 3, 4, or 5 mismatches; and
   the second guide oligonucleotide comprises a second spacer having a sequence identical or substantially identical to a guide 2 protospacer sequence listed in Table 2 or Table 5, or a 3′ portion thereof, with 0, 1, 2, 3, 4, or 5 mismatches,
   wherein the guide 1 protospacer and the guide 2 protospacer are in a same row of Table 2 or Table 5.
23. The pharmaceutical composition of any one of clauses 1 to 19, wherein
   the first guide oligonucleotide comprises a first spacer comprising a sequence identical to the 15, 16, 17, 18, 19, or twenty 3′-most nucleotides of a guide 1 protospacer sequence listed in Table 2 or Table 5; and
   the second guide oligonucleotide comprises a second spacer comprising a sequence identical to the 15, 16, 17, 18, 19, or twenty 3′-most nucleotides of a guide 2 protospacer sequence listed in Table 2 or Table 5,
   wherein the guide 1 protospacer and the guide 2 protospacer are in a same row of Table 2 or Table 5.
24. The pharmaceutical composition of any one of clauses 1 to 19, wherein
   the first guide oligonucleotide comprises a first spacer sequence identical to or substantially identical to a guide 1 protospacer listed in Table 5; and
   the second guide oligonucleotide comprises a second spacer sequence identical to or substantially identical to a corresponding (in a same row) guide 2 protospacer listed in Table 5.
25. The pharmaceutical composition of clause 24, wherein the first and second spacer sequences are identical to or substantially identical to protospacer sequences listed in Table 5 for which editing of 20% or greater was observed at 2500 ng/ml.
26. The pharmaceutical composition of clause 24, wherein the first and second spacer sequences are identical to or substantially identical to protospacer sequences listed in Table 5 for which editing of 30% or greater was observed at 2500 ng/ml.
27. The pharmaceutical composition of clause 24, wherein the first and second spacer sequences are identical to or substantially identical to protospacer sequences listed in Table 5 for which editing of 40% or greater was observed at 2500 ng/ml.
28. The pharmaceutical composition of clause 24, wherein the first and second spacer sequences are identical to or substantially identical to protospacer sequences listed in Table 5 for which editing of 50% or greater was observed at 2500 ng/ml.
29. The pharmaceutical composition of clause 24, wherein the first and second spacer sequences are identical to or substantially identical to protospacer sequences listed in Table 5 for which editing of 60% or greater was observed at 2500 ng/ml.
30. The pharmaceutical composition of clause 24, wherein the first and second spacer sequences are identical to or substantially identical to protospacer sequences listed in Table 5 for which editing of 70% or greater was observed at 2500 ng/ml.
31. The pharmaceutical composition of clause 24, wherein the first and second spacer sequences are identical to or substantially identical to protospacer sequences listed in Table 5 for which editing of 80% or greater was observed at 2500 ng/ml.
32. The pharmaceutical composition of clause 24, wherein the first and second spacer sequences are identical to or substantially identical to protospacer sequences listed in Table 5 for which editing of 85% or greater was observed at 2500 ng/ml.
33. The pharmaceutical composition of clause 24, wherein the first and second spacer sequences are identical to or substantially identical to protospacer sequences listed in Table 5 for which editing of 90% or greater was observed at 2500 ng/ml or where the guide pair corresponds to GA1264/GA1184, GA1265/GA1184, or GA1266/GA1184.
34. The pharmaceutical composition of any one of clauses 1 to 33, wherein at least one of the one or more polynucleotides is selected from any of the mRNA sequences listed in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the mRNA sequences listed in Table 1.
35. The pharmaceutical composition of any one of clauses 1 to 33, wherein at least one of the one or more polynucleotides comprises a coding sequence of any of the mRNA sequences listed in Table 1 or comprises a coding sequence having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the coding sequences of the SpCas9-D10A nickase mRNA sequences listed in Table 1.
36. The pharmaceutical composition of any one of clauses 1 to 35, wherein at least one of the one or more mRNAs that encodes the Cas nickase comprises:
   (a) a 5′ untranslated region (UTR);
   (b) a 3′ UTR region;
   (c) a poly(A) tail proximate to the 3′ UTR as compared to the 5′ UTR, said poly(A) tail comprising a chain of 80-150 nucleotides length that comprise adenine nucleotides; and
   (d) a gene editor coding region encoding an impaired CRISPR Cas9 endonuclease domain and a polymerase domain, said gene editor coding region extending between the 5′ UTR and the 3′ UTR.
37. The pharmaceutical composition of any one of clauses 1 to 36, wherein the scaffold region sequences of the first and second guide oligonucleotides (gRNAs) are each independently selected from one of the following sequences or a sequence having at least about 75%, at least about 80%, at least about 85%, at least about 90%, or at least about 95% identity to one of the following sequences:

5′-GUUUUAGAGCUAGAAAUAGCAAGUUAAAAUAAGGCUAGUCCGUUAUC
AACUUGAAAAAGUGGCACCGAGUCGGUGC-3′,
and
5′-GUUUGAGAGCUAUGCUGGAAACAGCAUAGCAAGUUCAAAUAAGGCUA
GUCCGUUAUCAACUUGAAAAAGUGGCACCGAGUCGGUGC-3′.

38. The pharmaceutical composition of any one of clauses 1 to 37, wherein at least one of the one or more Cas nickases is selected from:
   a Streptococcus pyogenes Cas9 variant, a Staphylococcus aureus Cas9 variant, or a Cas12a/Cpf1 variant.
39. The pharmaceutical composition of any one of the preceding clauses as it depends from clause 2, wherein the LNP is formulated with an mRNA:gRNA weight ratio of 1:1+/−5%, 10%, 15%, 20%, 30%, 40%, 50% 60%, 70%, 80%, 90% or 100% of the mRNA or gRNA or any therapeutically effective ratio.
40. The pharmaceutical composition of any one of the preceding clauses as it depends from clause 2 or 3, wherein the LNP has an N/P ratio from about 4 to about 7, with each ratio +/−5-20%,
41. The pharmaceutical composition of any one of the preceding clauses as it depends from clause 2 or 3, wherein the LNP has an N/P ratio from about 4 to about 7, about 4, about 4.5, about 5, about 5.5, or about 6, about 6.5, or about 7, with each ratio +/−5-20%.
42. The pharmaceutical composition of any one of the preceding clauses as it depends from clause 2 or 3, wherein the buffer solution containing the LNP has a pH of about 7.5+/−1.5 and comprises tris and/or sucrose.
43. The pharmaceutical composition of any one of the preceding clauses as it depends from clause 2 or 3, wherein the LNP has a mean diameter of about 70 nm+/−20 nm, 70 nm+/−10 nm, 70 nm+/−5 nm; 60 nm+/−20 nm, 60 nm+/−10 nm, 60 nm+/−5 nm; 50 nm+/−20 nm, 50 nm+/−10 nm, 50 nm+/−5 nm; 45 nm+/−20 nm, 45 nm+/−10 nm, 45 nm+/−5 nm.
44. The pharmaceutical composition according to any one of clauses 1 to 43, wherein administration of the composition to hepatocytes results in 40% or greater editing efficiency.
45. The pharmaceutical composition according to clause 44, wherein the hepatocytes are primary hepatocytes.
46. The pharmaceutical composition according to clause 44 or 45, wherein the hepatocytes are human hepatocytes.
47. The pharmaceutical composition according to any one of clauses 1 to 46, wherein administration of increasing doses of the composition to hepatocytes results in increased editing efficiency.
48. The pharmaceutical composition according to any one of clauses 1 to 47, wherein the composition is engineered to generate nicks on the first strand and the second strand of the LPA gene, wherein the distance between the nicks is 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or greater.
49. The pharmaceutical composition according to any one of clauses 1 to 48, wherein the composition is engineered to generate nicks on the first strand and the second strand of the LPA gene, wherein the distance between the nicks is between 20 and 50 nucleotides, such as between 23 and 45, between 30 and 40, between 31 and 40, between 32 and 40, between 33 and 40, between 31 and 39, between 32 and 39, between 33 and 39, or between 34 and 38 nucleotides.
50. A method for in vivo editing of an LPA gene in a mammalian subject comprising: administering to the subject a pharmaceutical composition comprising:
   (i) one or more polynucleotides (mRNAs) encoding one or more CRISPR Cas nickases,
   (ii) a first guide oligonucleotide (gRNA) comprising a first spacer sequence and a scaffold region; and
   (iii) a second guide oligonucleotide (gRNA) comprising a second spacer sequence and a scaffold region, and
   (iv) a delivery system that is engineered to deliver the one or more mRNAs, the first gRNA, and/or the second gRNA individually or collectively to the liver,
   wherein the first gRNA and at least one of the one or more Cas nickases are engineered to cause the at least one of the one or more Cas nickases to nick one of the first or second strands of the LPA gene at a first location of chromosome 6 from position 160,664,275 tol60,531,482, and
   wherein the second gRNA and at least one of the one or more Cas nickases are engineered to cause the at least one of the one or more Cas nickases to nick the other of the first or second strands of the LPA gene at a second location of chromosome 6 from position 160,664,275 tol60,531,482.
51. The method for in vivo editing of an LPA gene according to clause 50, wherein the delivery system comprises lipid nanoparticles (LNPs), wherein the LNPS comprise:
   (a) one or more ionizable lipids,
   (b) cholesterol,
   (c) one or more PEG-lipids,
   (d) a phospholipid; and
   (e) optionally including a targeting moiety, such as a GalNAc lipid.
52. The method for in vivo editing of an LPA gene according to clause 50 or 51, wherein the mammalian subject is a human.
53. The method for in vivo editing of an LPA gene according to any one of clauses 50 to 52, wherein the edited LPA gene is inactivated in the subject.
54. The method for in vivo editing of an LPA gene according to any one of clauses 50 to 53, wherein the method results in a lack of production of apo(a) protein or the production of non-functional apo(a) protein.
55. The method for in vivo editing of an LPA gene according to any one of clauses 50 to 54, wherein the method results in a reduced blood Lp(a) or apo(a) concentration.
56. The method for in vivo editing of an LPA gene according to any one of clauses 50 to 55, wherein at least one of the one or more Cas nickases, when in operative interaction with the first or second guide oligonucleotide, is engineered to nick the strand of the LPA gene opposite of the strand to which the operative guide oligonucleotide (gRNA) is hybridized.
57. The method for in vivo editing of an LPA gene according to clause 56, wherein at least one of the one or more Cas nickases comprises a Streptococcus pyogenes Cas9 nickase bearing a H840A mutation.
58. The method for in vivo editing of an LPA gene according to any one of clauses 50 to 55, wherein at least one of the one or more Cas nickases, when in operative interaction with the first or second guide oligonucleotide (gRNA), is engineered to nick the same strand of the LPA gene to which the operative guide is hybridized.
59. The method for in vivo editing of an LPA gene according to clause 58, wherein at least one of the Cas nickases comprises a Streptococcus pyogenes Cas9 nickase bearing a D10A mutation.
60. The method for in vivo editing of an LPA gene according to any one of clauses 50 to 59, wherein the sequences of the first and second scaffold region are the same.
61. The method for in vivo editing of an LPA gene according to any one of clauses 50 to 60, wherein the first spacer sequence of the first guide oligonucleotide (gRNA) comprises about 15 to about 26 nucleotides and has a sequence that is identical to or substantially identical to a targeted first protospacer sequence adjacent to a protospacer-adjacent motif (PAM) sequence on the first strand of the LPA gene.
62. The method for in vivo editing of an LPA gene according to any one of clauses 50 to 61, wherein the second spacer sequence of the second guide oligonucleotide (gRNA) comprises about 15 to about 26 nucleotides and has a sequence that is identical to or substantially identical to a second targeted protospacer sequence adjacent to a protospacer-adjacent motif (PAM) sequence on the second strand of the LPA gene.
63. The method for in vivo editing of an LPA gene according to any one of clauses 50 to 62, wherein the one or more Cas nickase and the first and second gRNAs are engineered to effect an indel or non-synonymous variant.
64. The method for in vivo editing of an LPA gene according to any one of clauses 50 to 63, wherein the first spacer sequence of the first guide oligonucleotide (gRNA) has a sequence that is identical to or substantially identical to a first protospacer sequence adjacent to a first protospacer-adjacent motif (PAM) sequence on the first strand of the LPA gene, and the second spacer sequence of the second guide oligonucleotide (gRNA) has a sequence that is identical to or substantially identical to a second protospacer sequence adjacent to a second protospacer-adjacent motif (PAM) sequence on the second strand of the LPA gene, and wherein the one or more Cas nickases, in operation with the first and second guide oligonucleotides, nicks opposing strands of the LPA gene between the first and second protospacer-adjacent motifs (PAMs) in a “PAM-out” configuration.
65. The method for in vivo editing of an LPA gene according to any one of clauses 50 to 63, wherein the first spacer sequence of the first guide oligonucleotide (gRNA) has a sequence that is identical to or substantially identical to a first protospacer sequence adjacent to a first protospacer-adjacent motif (PAM) sequence on the first strand of the LPA gene, and the second spacer sequence of the second guide oligonucleotide (gRNA) has a sequence that is identical to or substantially identical to a second protospacer sequence adjacent to a second protospacer-adjacent motif (PAM) sequence on the second strand of the LPA gene, and
   wherein the one or more Cas nickases, in operation with the first and second guide oligonucleotides, nicks opposing strands of the LPA gene outside the first and second protospacer-adjacent motifs (PAMs) in a “PAM-in” configuration.
66. The method for in vivo editing of an LPA gene according to any one of clauses 50 to 65, wherein the one or more mRNAs encode a single CRISPR Cas nickase.
67. The method for in vivo editing of an LPA gene according to any one of clauses 50 to 66, wherein the one or more Cas nickases in operation with the first and second guide oligonucleotides (gRNAs) are engineered to be directed to first and second target sequences opposite of respective first and second protospacer sequences within or in proximity to exon 25 of the LPA gene, which spans from chromosome 6, position 160,586,630, to chromosome 6, position 160,586,449, and to nick opposing strands of the LPA gene to effect indel variants and/or non-synonymous variants in the LPA gene.
68. The method for in vivo editing of an LPA gene according to any one of clauses 50 to 66, wherein the one or more Cas nickases in operation with the first and second guide oligonucleotides (gRNAs) are engineered to be directed to first and second target sequences opposite of respective first and second protospacer sequences within or in proximity to exon 19 of the LPA gene, which spans from chromosome 6, position 160,601,098, to chromosome 6, position 160,600,917, and to nick opposing strands of the LPA gene to effect indel variants and/or non-synonymous variants in the LPA gene.
69. The method for in vivo editing of an LPA gene according to any one of clauses 50 to 66, wherein the one or more Cas nickases in operation with the first and second guide oligonucleotides (gRNAs) are engineered to be directed to first and second target sequences opposite of respective first and second protospacer sequences within or in proximity to exon 31 of the LPA gene, which spans from chromosome 6, position 160,548,659, to chromosome 6, position 160,548,478, and to nick opposing strands of the LPA gene and to effect indel variants and/or non-synonymous variants in the LPA gene.
70. The method for in vivo editing of an LPA gene according to any one of clauses 50 to 69, wherein at least one of the one or more polynucleotides is selected from any of the mRNA sequences listed in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the mRNA sequences listed in Table 1.
71. The method for in vivo editing of an LPA gene according to any one of clauses 50 to 70, wherein at least one of the one or more polynucleotides comprises a coding sequence of any of the mRNA sequences listed in Table 1 or comprises a coding sequence having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the coding sequences of the SpCas9-D10A nickase mRNA sequences listed in Table 1.
72. The method for in vivo editing of an LPA gene according to any one of clauses 50 to 71, wherein at least one of the one or more mRNAs that encodes the Cas nickase comprises:
   (a) a 5′ untranslated region (UTR);
   (b) a 3′ UTR region;
   (c) a poly(A) tail proximate to the 3′ UTR as compared to the 5′ UTR, said poly(A) tail comprising a chain of 80-150 nucleotides length that comprise adenine nucleotides; and
   (d) a gene editor coding region encoding an impaired CRISPR Cas endonuclease domain and a polymerase domain, said gene editor coding region extending between the 5′ UTR and the 3′ UTR.
73. The method for in vivo editing of an LPA gene according to any one of clauses 50 to 72, wherein the scaffold region sequences of the first and second guide oligonucleotides (gRNA) are each independently selected from one of the following sequences or a sequence having at least about 75%, at least about 80%, at least about 85%, at least about 90%, or at least about 95% identity to one of the following sequences:

5′-GUUUUAGAGCUAGAAAUAGCAAGUUAAAAUAAGGCUAGUCCGUUAUC
AACUUGAAAAAGUGGCACCGAGUCGGUGC-3′,
and
5′-GUUUGAGAGCUAUGCUGGAAACAGCAUAGCAAGUUCAAAUAAGGCUA
GUCCGUUAUCAACUUGAAAAAGUGGCACCGAGUCGGUGC-3′.

74. The method for in vivo editing of an LPA gene according to any one of clauses 50 to 73,
   wherein the first guide oligonucleotide comprises a first spacer having a sequence identical to, or at least about 75%, at least about 80%, at least about 85%, at least about 90%, or at least about 95% identity to, a protospacer listed in Table 2 or Table 5; and
   the second guide oligonucleotide comprises a second spacer having a sequence identical to, or at least about 75%, at least about 80%, at least about 85%, at least about 90%, or at least about 95% identity to, a protospacer listed in Table 2 or Table 5,
   wherein the first spacer corresponds to a protospacer on one strand of the LPA gene and the second spacer corresponds to a protospacer on the other strand of the LPA gene.
75. The method for in vivo editing of an LPA gene according to any one of clauses 50 to 73, wherein
   the first guide oligonucleotide comprises a first spacer having a sequence identical to, or at least about 75%, at least about 80%, at least about 85%, at least about 90%, or at least about 95% identity to, a guide 1 protospacer listed in Table 2 or Table 5; and
   the second guide oligonucleotide comprises a second spacer having a sequence identical to, or at least about 75%, at least about 80%, at least about 85%, at least about 90%, or at least about 95% identity to, a guide 2 protospacer listed in Table 2 or Table 5,
   wherein the guide 1 protospacer and the guide 2 protospacer are in a same row of Table 2 or Table 5.
76. The method for in vivo editing of an LPA gene according to any one of clauses 50 to 73, wherein
   the first guide oligonucleotide comprises a first spacer having a sequence identical or substantially identical to a guide 1 protospacer sequence listed in Table 2 or Table 5, or a 3′ portion thereof, with 0, 1, 2, 3, 4, or 5 mismatches; and
   the second guide oligonucleotide comprises a second spacer having a sequence identical or substantially identical to a guide 2 protospacer sequence listed in Table 2 or Table 5, or a 3′ portion thereof, with 0, 1, 2, 3, 4, or 5 mismatches,
   wherein the guide 1 protospacer and the guide 2 protospacer are in a same row of Table 2 or Table 5.
77. The method for in vivo editing of an LPA gene according to any one of clauses 50 to 73, wherein
   the first guide oligonucleotide comprises a first spacer comprising a sequence identical to the 15, 16, 17, 18, 19, or twenty 3′-most nucleotides of a guide 1 protospacer sequence listed in Table 2 or Table 5; and
   the second guide oligonucleotide comprises a second spacer comprising a sequence identical to the 15, 16, 17, 18, 19, or twenty 3′-most nucleotides of a guide 2 protospacer sequence listed in Table 2 or Table 5,
   wherein the guide 1 protospacer and the guide 2 protospacer are in a same row of Table 2 or Table 5.
78. The method for in vivo editing of an LPA gene according to any one of clauses 50 to 73, wherein:
   the first guide oligonucleotide comprises a first spacer sequence identical to or substantially identical to a guide 1 protospacer listed in Table 5; and
   the second guide oligonucleotide comprises a second spacer sequence identical to or substantially identical to a corresponding (in a same row) guide 2 protospacer listed in Table 5.
79. The method for in vivo editing of an LPA gene according to clause 78, wherein the first and second spacer sequences are identical to or substantially identical to protospacer sequences listed in Table 5 for which editing of 20% or greater was observed at 2500 ng/ml.
80. The method for in vivo editing of an LPA gene according to clause 78, wherein the first and second spacer sequences are identical to or substantially identical to protospacer sequences listed in Table 5 for which editing of 30% or greater was observed at 2500 ng/ml.
81. The method for in vivo editing of an LPA gene according to clause 78, wherein the first and second spacer sequences are identical to or substantially identical to protospacer sequences listed in Table 5 for which editing of 40% or greater was observed at 2500 ng/ml.
82. The method for in vivo editing of an LPA gene according to clause 78, wherein the first and second spacer sequences are identical to or substantially identical to protospacer sequences listed in Table 5 for which editing of 50% or greater was observed at 2500 ng/ml.
83. The method for in vivo editing of an LPA gene according to clause 78, wherein the first and second spacer sequences are identical to or substantially identical to protospacer sequences listed in Table 5 for which editing of 60% or greater was observed at 2500 ng/ml.
84. The method for in vivo editing of an LPA gene according to clause 78, wherein the first and second spacer sequences are identical to or substantially identical to protospacer sequences listed in Table 5 for which editing of 70% or greater was observed at 2500 ng/ml.
85. The method for in vivo editing of an LPA gene according to clause 78, wherein the first and second spacer sequences are identical to or substantially identical to protospacer sequences listed in Table 5 for which editing of 80% or greater was observed at 2500 ng/ml.
86. The method for in vivo editing of an LPA gene according to clause 78, wherein the first and second spacer sequences are identical to or substantially identical to protospacer sequences listed in Table 5 for which editing of 85% or greater was observed at 2500 ng/ml.
87. The method for in vivo editing of an LPA gene according to clause 78, wherein the first and second spacer sequences are identical to or substantially identical to protospacer sequences listed in Table 5 for which editing of 90% or greater was observed at 2500 ng/ml or where the guide pair corresponds to GA1264/GA1184, GA1265/GA1184, or GA1266/GA1184.
88. The method for in vivo editing of an LPA gene according to any one of clauses 50 to 87, wherein at least one of the one or more Cas nickase is selected from:
   a Streptococcus pyogenes Cas9 variant, a Staphylococcus aureus Cas9 variant, or a Cas12a/Cpf1 variant.
89. The method for in vivo editing of an LPA gene according to 52 to 88 as it depends from clause 51, wherein the LNP is formulated with an mRNA:gRNA weight ratio of 1:1+/−5%, 10%, 15%, 20%, 30%, 40%, 50% 60%, 70%, 80%, 90% or 100% of the mRNA or gRNA or any therapeutically effective ratio.
90. The method for in vivo editing of an LPA gene according to clause 89, wherein the LNP has an N/P ratio from about 4 to about 7, about 4, about 4.5, about 5, about 5.5, or about 6, about 6.5, or about 7, with each ratio +/−5-20%,
91. The method for in vivo editing of an LPA gene according to clause 89 or 90, wherein the buffer solution containing the LNP has a pH of about 7.5+/−1.5 and comprises tris and/or sucrose.
92. The method for in vivo editing of an LPA gene according to any one of clauses 89 to 91, wherein the LNP has a mean diameter of about 70 nm+/−20 nm, 70 nm+/−10 nm, 70 nm+/−5 nm; 60 nm+/−20 nm, 60 nm+/−10 nm, 60 nm+/−5 nm; 50 nm+/−20 nm, 50 nm+/−10 nm, 50 nm+/−5 nm; 45 nm+/−20 nm, 45 nm+/−10 nm, 45 nm+/−5 nm.
93. A method for inactivating the LPA gene in vivo in a mammalian subject to treat and prevent cardiovascular disease comprising the step of: administering to the subject a pharmaceutical composition of any one of clauses 1 to 49.
94. A method for reducing blood Lp(a) concentration in a mammalian subject to treat and prevent cardiovascular disease comprising the step of: administering to the subject a pharmaceutical composition of any one of clauses 1 to 49.
95. A method for treating and/or preventing cardiovascular disease associated with the LPA gene in a mammalian subject comprising the step of: administering to the subject a pharmaceutical composition of any one of clauses 1 to 49.
96. A gene editing system for editing the LPA gene produced by expressing in a cell one or more exogenous polynucleotides (mRNA) encoding one or more CRISPR Cas nickases and introducing first and second gRNAs into the cell,
   wherein the first guide oligonucleotide (gRNA) comprises (i) a first spacer sequence that is complementary to a first strand of the LPA gene at a first target sequence and (ii) a first scaffold region that serves as a binding scaffold for at least one of the one or more Cas nickases,
   wherein the second guide oligonucleotide (gRNA) comprises (i) a second spacer sequence that is complementary to a second strand of the LPA gene at a second target sequence and (ii) a second scaffold region that serves as a binding scaffold for at least one of the one or more Cas nickases,
   wherein the first gRNA and the at least one of the one or more Cas nickases are engineered to cause the at least one of the one or more Cas nickases to nick one of the first or second strands of the LPA gene at a first location of chromosome 6 from position 160,664,275 to 160,531,482, and
   wherein the second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.
97. A method comprising:
   formulating a delivery system comprising one or more exogenous polynucleotides (mRNA) encoding one or more CRISPR Cas nickases, a first guide oligonucleotide (gRNA), and a second guide oligonucleotide (gRNA),
   wherein the first guide oligonucleotide (gRNA) comprises (i) a first spacer sequence that is complementary to a first strand of the LPA gene at a first target sequence and (ii) a first scaffold region that serves as a binding scaffold for at least one of the one or more Cas nickases,
   wherein the second guide oligonucleotide (gRNA) comprises (i) a second spacer sequence that is complementary to a second strand of the LPA gene at a second target sequence and (ii) a second scaffold region that serves as a binding scaffold for at least one of the one or more Cas nickases,
   wherein the first gRNA and the at least one of the one or more Cas nickases are engineered to cause the at least one of the one or more Cas nickases to nick one of the first or second strands of the LPA gene at a first location of chromosome 6 from position 160,664,275 to 160,531,482, and
   wherein the second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.
98. The method of clause 97, further comprising delivering the delivery system to cell.
99. The method of clause 98, wherein the cell is a primary human hepatocyte.
100. The method of clause 99, wherein delivering the delivery system to the primary human hepatocyte comprises administering the delivery system to a subject comprising the primary human hepatocyte.
101. A gene editing system comprising:
   a means for expressing one or more CRISPR Cas nickases in a cell; and
   a means for directing the one or more Cas nickases to first and second locations in the LPA gene and to cause the one or more Cas nickases to introduce a nick in a first strand of the LPA gene and to introduce a nick in a second strand of the LPA gene.
102. A pharmaceutical composition comprising the gene editing system of clause 100 and a delivery system.
103. The pharmaceutical composition of clause 102, wherein the delivery system comprises means for delivering to the cell the means for expressing one or more CRISPR Cas nickases and the means for directing the one or more Cas nickases to first and second locations in the LPA gene.
104. A gene editing system comprising:
   wherein the first guide oligonucleotide comprises a first scaffold region that serves as a binding scaffold for a nickase and comprises a first spacer having a sequence identical to, or at least about 75%, at least about 80%, at least about 85%, at least about 90%, or at least about 95% identity to, a protospacer listed in Table 2 or Table 5; and
   the second guide oligonucleotide comprises a second scaffold region that serves as a binding scaffold for the nickase and comprises a second spacer having a sequence identical to, or at least about 75%, at least about 80%, at least about 85%, at least about 90%, or at least about 95% identity to, a protospacer listed in Table 2 or Table 5,
   wherein the first spacer corresponds to a protospacer on one strand of the LPA gene and the second spacer corresponds to a protospacer on the other strand of the LPA gene.
105. A gene editing system comprising:
   a first guide oligonucleotide comprising a first scaffold region that serves as a binding scaffold for a nickase and comprising a first spacer having a sequence identical to, or at least about 75%, at least about 80%, at least about 85%, at least about 90%, or at least about 95% identity to, a guide 1 protospacer listed in Table 2 or Table 5; and
   a second guide oligonucleotide comprising a second scaffold region that serves as a binding scaffold for the nickase and comprising a second spacer having a sequence identical to, or at least about 75%, at least about 80%, at least about 85%, at least about 90%, or at least about 95% identity to, a guide 2 protospacer listed in Table 2 or Table 5,
   wherein the guide 1 protospacer and the guide 2 protospacer are in a same row of Table 2 or Table 5.
106. A gene editing system comprising:
   a first guide oligonucleotide comprising a first scaffold region that serves as a binding scaffold for a nickase and comprising a first spacer having a sequence identical or substantially identical to a guide 1 protospacer sequence listed in Table 2 or Table 5, or a 3′ portion thereof, with 0, 1, 2, 3, 4, or 5 mismatches; and
   a second guide oligonucleotide comprising a second scaffold region that serves as a binding scaffold for the nickase and comprising a second spacer having a sequence identical or substantially identical to a guide 2 protospacer sequence listed in Table 2 or Table 5, or a 3′ portion thereof, with 0, 1, 2, 3, 4, or 5 mismatches,
   wherein the guide 1 protospacer and the guide 2 protospacer are in a same row of Table 2 or Table 5.
107. A gene editing system comprising:
   a first guide oligonucleotide comprising a first scaffold region that serves as a binding scaffold for a nickase and comprising a first spacer comprising a sequence identical to the 15, 16, 17, 18, 19, or twenty 3′-most nucleotides of a guide 1 protospacer sequence listed in Table 2 or Table 5; and
   a second guide oligonucleotide comprising a second spacer comprising a second scaffold region that serves as a binding scaffold for the nickase and comprising a sequence identical to the 15, 16, 17, 18, 19, or twenty 3′-most nucleotides of a guide 2 protospacer sequence listed in Table 2 or Table 5,
   wherein the guide 1 protospacer and the guide 2 protospacer are in a same row of Table 2 or Table 5.
108. A gene editing system comprising:
   a first guide oligonucleotide having a first scaffold region that serves as a binding scaffold for a nickase and comprising a first spacer sequence identical to or substantially identical to a guide 1 protospacer listed in Table 5; and
   a second guide oligonucleotide having a second scaffold region that serves as a binding scaffold for the nickase and comprising a second spacer sequence identical to or substantially identical to a corresponding (in a same row) guide 2 protospacer listed in Table 5.
109. The gene editing system of clause 108, wherein the first and second spacer sequences are identical to or substantially identical to protospacer sequences listed in Table 5 for which editing of 20% or greater was observed at 2500 ng/ml.
110. The gene editing system of clause 108, wherein the first and second spacer sequences are identical to or substantially identical to protospacer sequences listed in Table 5 for which editing of 30% or greater was observed at 2500 ng/ml.
111. The gene editing system of clause 108, wherein the first and second spacer sequences are identical to or substantially identical to protospacer sequences listed in Table 5 for which editing of 40% or greater was observed at 2500 ng/ml.
112. The gene editing system of clause 108, wherein the first and second spacer sequences are identical to or substantially identical to protospacer sequences listed in Table 5 for which editing of 50% or greater was observed at 2500 ng/ml.
113. The gene editing system of clause 108, wherein the first and second spacer sequences are identical to or substantially identical to protospacer sequences listed in Table 5 for which editing of 60% or greater was observed at 2500 ng/ml.
114. The gene editing system of clause 108, wherein the first and second spacer sequences are identical to or substantially identical to protospacer sequences listed in Table 5 for which editing of 70% or greater was observed at 2500 ng/ml.
115. The gene editing system of clause 108, wherein the first and second spacer sequences are identical to or substantially identical to protospacer sequences listed in Table 5 for which editing of 80% or greater was observed at 2500 ng/ml.
116. The gene editing system of clause 108, wherein the first and second spacer sequences are identical to or substantially identical to protospacer sequences listed in Table 5 for which editing of 85% or greater was observed at 2500 ng/ml.
117. The gene editing system of clause 108, wherein the first and second spacer sequences are identical to or substantially identical to protospacer sequences listed in Table 5 for which editing of 90% or greater was observed at 2500 ng/ml or where the guide pair corresponds to GA1264/GA1184, GA1265/GA1184, or GA1266/GA1184.
118. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CUGUCACCAGGCAUUGUGUC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-UGUCCUUGCAACUCUCACGG-3′.
119. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CUGUCACCAGGCAUUGUGUC-3′; and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-UGUCCUUGCAACUCUCACGG-3′.
120. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-GUAGUAGCAGUCCUGUACCC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CAUUAUGGACAGAGUUACCG-3′.
121. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-GUAGUAGCAGUCCUGUACCC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CAUUAUGGACAGAGUUACCG-3′.
122. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-AGGACACUCGAUUCUGUCAC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CACAACUCCCACAGUGGCCC-3′.
123. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-AGGACACUCGAUUCUGUCA-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CACAACUCCCACAGUGGCCC-3′.
124. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CUGUCACUGGACAUUGUGUC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-AAGUGUCCUUGCGACGUCCA-3′.
125. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CUGUCACUGGACAUUGUGUC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-AAGUGUCCUUGCGACGUCCA-3′.
126. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-GGAGCAAAGCCCCACAGUCC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-GUUGGUGCUGAAAUUCAAAG-3′.
127. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-GGAGCAAAGCCCCACAGUCC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-GUUGGUGCUGAAAUUCAAAG-3′.
128. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-GGAGCAAAGCCCCGGGGUCC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-GUUGGUGCUGAAAUUCAAAG-3′.
129. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-GGAGCAAAGCCCCGGGGUCC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-GUUGGUGCUGAAAUUCAAAG-3′.
130. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CUGGAACUGGGACCACCGU-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-ACAGAGCUUCCUUCUGAAGA-3′.
131. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CUGGAACUGGGACCACCGU-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-ACAGAGCUUCCUUCUGAAGA-3′.
132. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-AUGCCAGUGUGGUGUCAUAG-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-ACCACAGAAUACUACCCAAA-3′.
133. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-AUGCCAGUGUGGUGUCAUAG-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-ACCACAGAAUACUACCCAAA-3′.
134. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-GGAGCCAGAAUAACAUUCGG-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CUAGAGGCUIUIUUUUUGAACA-3′.
135. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-GGAGCCAGAAUAACAUUCGG-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CUAGAGGCUUUUUUUGAACA-3′.
136. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CAGAUGCUGAGAUUAGUCCU-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-UGGAUUCCUGCAGUAGUUCC-3′.
137. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CAGAUGCUGAGAUUAGUCCU-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-UGGAUUCCUGCAGUAGUUCC-3′.
138. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-UGACACCACAUUGGCAUCGG-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-ACAUGUUCUUCCUGUGAUAG-3′.
139. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-UGACACCACAUUGGCAUCGG-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-ACAUGUUCUUCCUGUGAUAG-3′.
140. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CAUAGAUGACCAAGAUUGAC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-UGAUACCACACUGGCAUCAG-3′.
141. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CAUAGAUGACCAAGAUUGAC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-UGAUACCACACUGGCAUCAG-3′.
142. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CCAUCACUGGACAUUGCGUC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-AACUCUCCUCACAACUCCCA-3′.
143. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CCAUCACUGGACAUUGCGUC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-AACUCUCCUCACAACUCCCA-3′.
144. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CUGCAUCUGAGCAUCGUGUC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CGUCCCUCCGAAUGUUAUUC-3′.
145. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CUGCAUCUGAGCAUCGUGUC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CGUCCCUCCGAAUGUUAUUC-3′.
146. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-AAACAGCCGUGGACGUCGCA-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-UGAACAAGGUAAGAAGUCUC-3′.
147. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-AAACAGCCGUGGACGUCGCA-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-UGAACAAGGUAAGAAGUCUC-3′.
148. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-ACAGAGGCUCCUUCUGAACA-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-GCUUGGAACCGGGGCCACUG-3′.
149. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-ACAGAGGCUCCUUCUGAACA-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-GCUUGGAACCGGGGCCACUG-3′.
150. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-AUGCCAGUGUGGUGUCAUAG-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-ACAACAGAAUAUUAUCCAAA-3′.
151. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-AUGCCAGUGUGGUGUCAUAG-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-ACAACAGAAUAUUAUCCAAA-3′.
152. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-CUAUGACACCACAUUGGCAU-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-ACAUGUUCUUCCUGUGAUAG-3′.
153. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-CUAUGACACCACAUUGGCAU-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-ACAUGUUCUUCCUGUGAUAG-3′.
154. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-AAUAACAUUCGGAGGGACGA-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-UAUUCUGGCUCCAAGCCUAG-3′.
155. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-AAUAACAUUCGGAGGGACGA-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-UAUUCUGGCUCCAAGCCUAG-3′.
156. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-GUAGCAGUCCUGUACCCCGG-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CAUUAUGGACAGAGUUACCG-3′.
157. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-GUAGCAGUCCUGUACCCCGG-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CAUUAUGGACAGAGUUACCG-3′.
158. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-AGUAGCAGUCCUGUACCCCG-3′, and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CAUUAUGGACAGAGUUACCG-3′.
159. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-AGUAGCAGUCCUGUACCCCG-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CAUUAUGGACAGAGUUACCG-3′.
160. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-UAGUAGCAGUCCUGUACCCC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-CAUUAUGGACAGAGUUACCG-3′.
161. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-UAGUAGCAGUCCUGUACCCC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-CAUUAUGGACAGAGUUACCG-3′.
162. A gene editing system comprising:
   a first guide oligonucleotide comprises a first scaffold region that serves as a binding scaffold for a nickase and comprises a spacer sequence comprising nucleotides 6 to 20, 5 to 20, 4 to 20, 3 to 20, 2 to 20, or 1 to 20 of the following sequence 5′-UGGACCACAUGGCUUUGCUC-3′; and
   a second guide oligonucleotide comprises a second scaffold region that serves as a binding scaffold for the nickase and comprises a spacer sequence comprising 6 to 20, 5 to 20, 4 to 20, 3 to 20, 2 to 20, or 1 to 20 of the following sequence 5′-ACGUACUCCACCACUGUCAC-3′.
163. A gene editing system comprising:
   a first guide oligonucleotide comprises a first scaffold region that serves as a binding scaffold for a nickase and comprises a spacer having a sequence that is at least about 75%, at least about 80%, at least about 85%, at least about 90%, at least about 95% identical to, or is identical to 5′-UGGACCACAUGGCUUUGCUC-3′, and
   a second guide oligonucleotide comprises a second scaffold region that serves as a binding scaffold for the nickase and comprises a spacer having a sequence that is at least about 75%, at least about 80%, at least about 85%, at least about 90%, at least about 95% identical to, or is identical to 5′-ACGUACUCCACCACUGUCAC-3′.
164. One or more nucleic acids encoding the first guide oligonucleotide and the second guide oligonucleotide of any one of clauses 104 to 163.
165. A gene editing system comprising the one or more nucleic acids of clause 164.
166. The gene editing system of any one of clauses 104 to 163 or 165, further comprising a gene editor or a nucleic acid encoding the gene editor.
167. The gene editing system of clause 166, wherein the gene editor comprises a Cas nickase or a nucleic acid encoding the Cas nickase.
168. A gene editing system for editing the LPA gene comprising:
   a Cas nickase or a nucleic acid encoding the Cas nickase;
   a first guide oligonucleotide comprising (i) a first spacer sequence that is complementary to a first strand of the LPA gene at a first target sequence and (ii) a first scaffold region that serves as a binding scaffold for the Cas nickase;
   a second guide oligonucleotide comprising (i) a second spacer sequence that is complementary to a second strand of the LPA gene at a second target sequence and (ii) a second scaffold region that serves as a binding scaffold for the Cas nickase,
   wherein the first guide oligonucleotide and the Cas nickase are engineered to cause the Cas nickase to nick one of the first or second strands of the LPA gene at a first location,
   wherein the second guide oligonucleotide and the Cas nickase are engineered to cause the Cas nickase to nick the other of the first or second strands of the LPA gene at a second location, and
   wherein the first location and the second location are spaced apart by a distance of 1 to 200 nucleotides.
169. The gene editing system of clause 168, wherein the first location and the second location are spaced apart by a distance of 20 to 50 nucleotides.
170. A gene editing system, pharmaceutical composition, or method of any one of the preceding clauses, wherein the first guide oligonucleotide and/or the second guide oligonucleotide comprises a modified nucleotide.
171. The gene editing system, pharmaceutical composition, or method of clause 170, wherein
   the first guide oligonucleotide and/or the second guide oligonucleotide comprises a modified sugar moiety.
172. The gene editing system, pharmaceutical composition, or method of clause 171, wherein the modified sugar moiety comprises a 2′-O-methyl, 2′-O-methoxyethyl, 2′-O-aminoethyl, 2′-Fluoro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, or bicyclic modified sugar.
173. The gene editing system, pharmaceutical composition, or method of clause 171, wherein the modified nucleotide comprises a 2′-O-methyl modification.
174. The gene editing system, pharmaceutical composition, or method of any one of clauses 170 to 173, wherein the first guide oligonucleotide and/or the second guide oligonucleotide comprises a backbone modification.
175. The gene editing system, pharmaceutical composition, or method of clause 174, wherein the backbone modification comprises a phosphorothioate, a phosphorodithioate, a phosphoroselenoate, a phosphorodiselenoate, a phosphoroanilothioate, a phosphoraniladate, a phosphoramidate, or a phosphorodiamidate linkage.
176. The gene editing system, pharmaceutical composition, or method of clause 174, wherein the backbone modification comprises a phosphorothioate linkage.
177. The gene editing system, pharmaceutical composition, or method of any one of clauses 170 to 176, wherein the first spacer sequence and/or the second spacer sequence comprises from 1 to 20 modified nucleotides.
178. The gene editing system, pharmaceutical composition, or method of any one of clauses 170 to 176, wherein the first spacer sequence and/or the second spacer sequence comprises from 1 to 10 modified nucleotides.
179. The gene editing system, pharmaceutical composition, or method of any one of clauses 170 to 176, wherein the first spacer sequence and/or the second spacer sequence comprises from 1 to 5 modified nucleotides.
180. The gene editing system, pharmaceutical composition, or method of any one of clauses 170 to 176, wherein the first spacer sequence and/or the second spacer sequence comprises from 1 to 3 modified nucleotides.
181. The gene editing system, pharmaceutical composition, or method of any one of clauses 170 to 176, wherein the first spacer sequence and/or the second spacer sequence comprises 3 modified nucleotides.
182. The gene editing system, pharmaceutical composition, or method of any one of clauses 170 to 181, wherein one or more of the five 5′-most nucleotides of the first and/or second spacer sequence are modified.
183. The gene editing system, pharmaceutical composition, or method of 182, wherein one or more of the five 5′-most nucleotides of the first and/or second spacer sequence are modified to include a 2′-O-methyl group, a phosphorothioate linkage, or a combination thereof.
184. The gene editing system, pharmaceutical composition, or method of any one of clauses 170 to 181, wherein one or more of the three 5′-most nucleotides of the first and/or second spacer sequence are modified.
185. The gene editing system, pharmaceutical composition, or method of 184, wherein one or more of the three 5′-most nucleotides of the first and/or second spacer sequence are modified to include a 2′-O-methyl group, a phosphorothioate linkage, or a combination thereof.
186. The gene editing system, pharmaceutical composition, or method of any one of clauses 170 to 181, wherein the three 5′-most nucleotides of the first and/or second spacer sequence are modified.
187. The gene editing system, pharmaceutical composition, or method of 186, wherein the three 5′-most nucleotides of the first and/or second spacer sequence are modified to include a 2′-O-methyl group, a phosphorothioate linkage, or a combination thereof.
188. The gene editing system, pharmaceutical composition, or method of any one of clauses 170 to 187, wherein the scaffold sequence of the first and/or second guide oligonucleotide comprises from 20 to 70 modified nucleotides.
189. The gene editing system, pharmaceutical composition, or method of any one of clauses 170 to 187, wherein the scaffold sequence of the first and/or second guide oligonucleotide comprises from 30 to 65 modified nucleotides.
190. The gene editing system, pharmaceutical composition, or method of any one of clauses 170 to 187, wherein the scaffold sequence of the first and/or second guide oligonucleotide comprises from 40 to 60 modified nucleotides.
191. The gene editing system, pharmaceutical composition, or method of any one of clauses 170 to 187, wherein the scaffold sequence of the first and/or second guide oligonucleotide comprises from 50 to 55 modified nucleotides.
192. The gene editing system, pharmaceutical composition, or method of any one of clauses 170 to 187, wherein from 30% to 90% of the nucleotides of the scaffold sequence of the first and/or second guide oligonucleotide are modified.
193. The gene editing system, pharmaceutical composition, or method of any one of clauses 170 to 187, wherein from 45% to 80% of the nucleotides of the scaffold sequence of the first and/or second guide oligonucleotide are modified.
194. The gene editing system, pharmaceutical composition, or method of any one of clauses 170 to 187, wherein from 60% to 75% of the nucleotides of the scaffold sequence of the first and/or second guide oligonucleotide are modified.
195. The gene editing system, pharmaceutical composition, or method of any one of clauses 170 to 187, wherein from 65% to 75% of the nucleotides of the scaffold sequence of the first and/or second guide oligonucleotide are modified.
196. The gene editing system, pharmaceutical composition, or method of any one of clauses 170 to 195, wherein the scaffold sequence of the first and/or second guide oligonucleotide comprises a nucleotide having a 2′ sugar modification.
197. The gene editing system, pharmaceutical composition, or method of any clause 196,
   wherein from 30% to 90% of the nucleotides of the scaffold sequence of the first and/or second guide oligonucleotide comprises a 2′ sugar modification.
198. The gene editing system, pharmaceutical composition, or method of any clause 196,
   wherein from 45% to 80% of the nucleotides of the scaffold sequence of the first and/or second guide oligonucleotide comprises a 2′ sugar modification.
199. The gene editing system, pharmaceutical composition, or method of any clause 196, wherein from 60% to 75% of the nucleotides of the scaffold sequence of the first and/or second guide oligonucleotide comprises a 2′ sugar modification.
200. The gene editing system, pharmaceutical composition, or method of any clause 196, wherein from 65% to 75% of the nucleotides of the scaffold sequence of the first and/or second guide oligonucleotide comprises a 2′ sugar modification.
201. The gene editing system, pharmaceutical composition, or method of any one of clauses 170 to 196, wherein the scaffold sequence of the first and/or second guide oligonucleotide comprises a nucleotide having a 2′-O-methyl group.
202. The gene editing system, pharmaceutical composition, or method of clause 201, wherein from 30% to 90% of the nucleotides of the scaffold sequence of the first and/or second guide oligonucleotide comprises a 2′-O-methyl group.
203. The gene editing system, pharmaceutical composition, or method of clause 201, wherein from 45% to 80% of the nucleotides of the scaffold sequence of the first and/or second guide oligonucleotide comprises a 2′-O-methyl group.
204. The gene editing system, pharmaceutical composition, or method of clause 201, wherein from 60% to 75% of the nucleotides of the scaffold sequence of the first and/or second guide oligonucleotide comprises a 2′-O-methyl group.
205. The gene editing system, pharmaceutical composition, or method of clause 201, wherein from 65% to 75% of the nucleotides of the scaffold sequence of the first and/or second guide oligonucleotide comprises a 2′-O-methyl group.
206. The gene editing system of any one of clauses 163 or 165 to 204, wherein the gene editor comprises a Cas9-H840A nickase or a nucleic acid encoding the Cas9-H840A nickase.
207. The gene editing system of clauses 206, wherein the gene editor comprises the nucleic acid encoding the Cas9-H840A nickase, and wherein the nucleic acid encoding the Cas9-H840A nickase is an mRNA comprising the sequence of SpCas9-H840A in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to the mRNA sequence of SpCas9-H840A listed in Table 1.
208. The gene editing system of any one of clauses 163 or 165 to 204, wherein the gene editor comprises a Cas9-D10A nickase or a nucleic acid encoding the Cas9-D10A nickase.
209. The gene editing system of any one of clauses 208, wherein the gene editor comprises the nucleic acid encoding the Cas9-D10A nickase, and wherein the nucleic acid encoding the Cas9-D10A nickase is an mRNA comprising the sequence of SpCas9-D10A in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to the mRNA sequence of SpCas9-D10A listed in Table 1.
210. The pharmaceutical composition, method, or gene editing system of any one of the preceding clauses, wherein the first guide oligonucleotide and/or the second guide oligonucleotide comprise a scaffold, wherein the scaffold comprises the following nucleotide sequence: 5′-GUUUUAGAGCUAGAAAUAGCAAGUUAAAAUAAGGCUAGUCCGUUAUCAAC UUGAAAAAGUGGCACCGAGUCGGUGC-3′.
211. The pharmaceutical composition, method, or gene editing system of clause 210, wherein the scaffold comprises one or more of the modified nucleotides in the following nucleotide sequence: 5′-mGUUUUAGmAmGmCmUmAGmAmAmAmUmAmGmCmAmAGUUmAAmAAmU AmAmGmGmCmUmAGUmCmCGUUAmUmCAAmCmUmUGmAmAmAmAmAmG mUmGGmCmAmCmCmGmAmGmUmCmGmGmUmGmC 3′, where m of mN is 2′-O-methyl ribose.
212. The pharmaceutical composition, method, or gene editing system of any one of the preceding clauses, wherein the first guide oligonucleotide and/or the second guide oligonucleotide comprise an RNA motif at the 5′ end of the guide oligonucleotide.
213. The pharmaceutical composition, method, or gene editing system of clause 212, wherein the RNA motif comprises, consists essentially of, or consists of 5′-UUU-3′.
214. The pharmaceutical composition, method, or gene editing system of clause 212, wherein the RNA motif comprises, consists essentially of, or consists of 5′-mU*mU*mU*mU-3′, where “mU*” indicates a phosphorothioated 2′-O-methyl uracil base, and “mU” indicates a 2′-O-methyl uracil base.
1A. A pharmaceutical composition for in vivo editing of an LPA gene, in a mammalian subject, comprising:
   (i) an engineered, non-naturally occurring gene editing system, which comprises:
      (a) one or more polynucleotides (mRNAs) encoding one or more nickases;
      (b) a first guide oligonucleotide (gRNA) comprising a first spacer sequence that is complementary to a first strand of the LPA gene at a first target sequence and a first scaffold region that serves as a binding scaffold for at least one of the one or more nickases; and
      (c) a second guide oligonucleotide (gRNA) comprising a second spacer sequence that is complementary to a second strand of the LPA gene at a second target sequence and a second scaffold region that serves as a binding scaffold for at least one of the one or more nickases, and
   (ii) a delivery system that is engineered to deliver the one or more mRNAs, the first gRNA, and/or the second gRNA individually or collectively to the liver,
   wherein the first gRNA and the at least one of the one or more nickases are engineered to cause the at least one of the one or more nickases to nick one of the first or second strands of the LPA gene at a first location of chromosome 6 from position 160,664,275 to 160,531,482,
   wherein the second gRNA and the at least one of the one or more nickase are engineered to cause the at least one of the one or more nickases to nick the other of the first or second strands of the LPA gene at a second location of chromosome 6 from position 160,664,275 to 160,531,482, and
   wherein the first spacer is homologous to a guide 1 protospacer listed in Table 2 or Table 5, and the second spacer is homologous to a guide 2 protospacer listed in Table 2 or Table 5.
2A. The pharmaceutical composition of clause 1A, wherein the first spacer has a sequence that is at least about 75%, at least about 80%, at least about 85%, at least about 90%, at least about 95% identical to, or is identical to the guide 1 protospacer listed in Table 2 or Table 5, and wherein the second spacer has a sequence that is at least about 75%, at least about 80%, at least about 85%, at least about 90%, at least about 95% identical to, or is identical to the guide 2 protospacer listed in Table 2 or Table 5.
3A. The pharmaceutical composition of clause 1A or 2A, wherein the first spacer comprises a sequence identical to or substantially identical to a guide 1 protospacer listed in Table 2 or Table 5, and the second spacer comprises a sequence identical to or substantially identical to a corresponding guide 2 protospacer (in the same row) listed in Table 2 or Table 5.
4A. The pharmaceutical composition of clause 1A,
   wherein the first spacer has a sequence that is at least about 75%, at least about 80%, at least about 85%, at least about 90%, at least about 95% identical to, or is identical to 5′-CUGUCACCAGGCAUUGUGUC-3′, and the second spacer has a sequence that is at least about 75%, at least about 80%, at least about 85%, at least about 90%, at least about 95% identical to, or is identical to 5′-UGUCCUUGCAACUCUCACGG-3′,
   wherein the first spacer has a sequence that is at least about 75%, at least about 80%, at least about 85%, at least about 90%, at least about 95% identical to, or is identical to 5′-GUAGUAGCAGUCCUGUACCC-3′, and the second spacer has a sequence that is at least about 75%, at least about 80%, at least about 85%, at least about 90%, at least about 95% identical to, or is identical to 5′-CAUUAUGGACAGAGUUACCG-3′,
   wherein the first spacer has a sequence that is at least about 75%, at least about 80%, at least about 85%, at least about 90%, at least about 95% identical to, or is identical to 5′-AGGACACUCGAUUCUGUCA-3′, and the second spacer has a sequence that is at least about 75%, at least about 80%, at least about 85%, at least about 90%, at least about 95% identical to, or is identical to 5′-CACAACUCCCACAGUGGCCC-3′,
   wherein the first spacer has a sequence that is at least about 75%, at least about 80%, at least about 85%, at least about 90%, at least about 95% identical to, or is identical to 5′-CUGUCACUGGACAUUGUGUC-3′, and the second spacer has a sequence that is at least about 75%, at least about 80%, at least about 85%, at least about 90%, at least about 95% identical to, or is identical to 5′-AAGUGUCCUUGCGACGUCCA-3′,
   wherein the first spacer has a sequence that is at least about 75%, at least about 80%, at least about 85%, at least about 90%, at least about 95% identical to, or is identical to 5′-GGAGCAAAGCCCCACAGUCC-3′, and the second spacer has a sequence that is at least about 75%, at least about 80%, at least about 85%, at least about 90%, at least about 95% identical to, or is identical to 5′-GUUGGUGCUGAAAUUCAAAG-3′,
   wherein the first spacer has a sequence that is at least about 75%, at least about 80%, at least about 85%, at least about 90%, at least about 95% identical to, or is identical to 5′-GGAGCAAAGCCCCGGGGUCC-3′, and the second spacer has a sequence that is at least about 75%, at least about 80%, at least about 85%, at least about 90%, at least about 95% identical to, or is identical to 5′-GUUGGUGCUGAAAUUCAAAG-3′,
   wherein the first spacer has a sequence that is at least about 75%, at least about 80%, at least about 85%, at least about 90%, at least about 95% identical to, or is identical to 5′-CUGGAACUGGGACCACCGU-3′, and the second spacer has a sequence that is at least about 75%, at least about 80%, at least about 85%, at least about 90%, at least about 95% identical to, or is identical to 5′-ACAGAGCUUCCUUCUGAAGA-3′,
   wherein the first spacer has a sequence that is at least about 75%, at least about 80%, at least about 85%, at least about 90%, at least about 95% identical to, or is identical to 5′-AUGCCAGUGUGGUGUCAUAG-3′, and the second spacer has a sequence that is at least about 75%, at least about 80%, at least about 85%, at least about 90%, at least about 95% identical to, or is identical to 5′-ACCACAGAAUACUACCCAAA-3′,
   wherein the first spacer has a sequence that is at least about 75%, at least about 80%, at least about 85%, at least about 90%, at least about 95% identical to, or is identical to 5′-GGAGCCAGAAUAACAUUCGG-3′, and the second spacer has a sequence that is at least about 75%, at least about 80%, at least about 85%, at least about 90%, at least about 95% identical to, or is identical to 5′-CUAGAGGCUUUUUUUGAACA-3′;
   wherein the first spacer has a sequence that is at least about 75%, at least about 80%, at least about 85%, at least about 90%, at least about 95% identical to, or is identical to 5′-CAGAUGCUGAGAUUAGUCCU-3′, and the second spacer has a sequence that is at least about 75%, at least about 80%, at least about 85%, at least about 90%, at least about 95% identical to, or is identical to 5′-UGGAUUCCUGCAGUAGUUCC-3′,
   wherein the first spacer has a sequence that is at least about 75%, at least about 80%, at least about 85%, at least about 90%, at least about 95% identical to, or is identical to 5′-UGACACCACAUUGGCAUCGG-3′, and the second spacer has a sequence that is at least about 75%, at least about 80%, at least about 85%, at least about 90%, at least about 95% identical to, or is identical to 5′-ACAUGUUCUUCCUGUGAUAG-3′,
   wherein the first spacer has a sequence that is at least about 75%, at least about 80%, at least about 85%, at least about 90%, at least about 95% identical to, or is identical to 5′-CAUAGAUGACCAAGAUUGAC-3′, and the second spacer has a sequence that is at least about 75%, at least about 80%, at least about 85%, at least about 90%, at least about 95% identical to, or is identical to 5′-UGAUACCACACUGGCAUCAG-3′,
   wherein the first spacer has a sequence that is at least about 75%, at least about 80%, at least about 85%, at least about 90%, at least about 95% identical to, or is identical to 5′-CCAUCACUGGACAUUGCGUC-3′, and the second spacer has a sequence that is at least about 75%, at least about 80%, at least about 85%, at least about 90%, at least about 95% identical to, or is identical to 5′-AACUCUCCUCACAACUCCCA-3′,
   wherein the first spacer has a sequence that is at least about 75%, at least about 80%, at least about 85%, at least about 90%, at least about 95% identical to, or is identical to 5′-CUGCAUCUGAGCAUCGUGUC-3′, and the second spacer has a sequence that is at least about 75%, at least about 80%, at least about 85%, at least about 90%, at least about 95% identical to, or is identical to 5′-CGUCCCUCCGAAUGUUAUUC-3′,
   wherein the first spacer has a sequence that is at least about 75%, at least about 80%, at least about 85%, at least about 90%, at least about 95% identical to, or is identical to 5′-AAACAGCCGUGGACGUCGCA-3′, and the second spacer has a sequence that is at least about 75%, at least about 80%, at least about 85%, at least about 90%, at least about 95% identical to, or is identical to 5′-UGAACAAGGUAAGAAGUCUC-3′,
   wherein the first spacer has a sequence that is at least about 75%, at least about 80%, at least about 85%, at least about 90%, at least about 95% identical to, or is identical to 5′-ACAGAGGCUCCUUCUGAACA-3′, and the second spacer has a sequence that is at least about 75%, at least about 80%, at least about 85%, at least about 90%, at least about 95% identical to, or is identical to 5′-GCUUGGAACCGGGGCCACUG-3′,
   wherein the first spacer has a sequence that is at least about 75%, at least about 80%, at least about 85%, at least about 90%, at least about 95% identical to, or is identical to 5′-AUGCCAGUGUGGUGUCAUAG-3′, and the second spacer has a sequence that is at least about 75%, at least about 80%, at least about 85%, at least about 90%, at least about 95% identical to, or is identical to 5′-ACAACAGAAUAUUAUCCAAA-3′,
   wherein the first spacer has a sequence that is at least about 75%, at least about 80%, at least about 85%, at least about 90%, at least about 95% identical to, or is identical to 5′-CUAUGACACCACAUUGGCAU-3′, and the second spacer has a sequence that is at least about 75%, at least about 80%, at least about 85%, at least about 90%, at least about 95% identical to, or is identical to 5′-ACAUGUUCUUCCUGUGAUAG-3′,
   wherein the first spacer has a sequence that is at least about 75%, at least about 80%, at least about 85%, at least about 90%, at least about 95% identical to, or is identical to 5′-AAUAACAUUCGGAGGGACGA-3′, and the second spacer has a sequence that is at least about 75%, at least about 80%, at least about 85%, at least about 90%, at least about 95% identical to, or is identical to 5′-UAUUCUGGCUCCAAGCCUAG-3′,
   wherein the first spacer has a sequence that is at least about 75%, at least about 80%, at least about 85%, at least about 90%, at least about 95% identical to, or is identical to 5′-GUAGCAGUCCUGUACCCCGG-3′, and the second spacer has a sequence that is at least about 75%, at least about 80%, at least about 85%, at least about 90%, at least about 95% identical to, or is identical to 5′-CAUUAUGGACAGAGUUACCG-3′,
   wherein the first spacer has a sequence that is at least about 75%, at least about 80%, at least about 85%, at least about 90%, at least about 95% identical to, or is identical to 5′-AGUAGCAGUCCUGUACCCCG-3′, and the second spacer has a sequence that is at least about 75%, at least about 80%, at least about 85%, at least about 90%, at least about 95% identical to, or is identical to 5′-CAUUAUGGACAGAGUUACCG-3′,
   wherein the first spacer has a sequence that is at least about 75%, at least about 80%, at least about 85%, at least about 90%, at least about 95% identical to, or is identical to 5′-UAGUAGCAGUCCUGUACCCC-3′, and the second spacer has a sequence that is at least about 75%, at least about 80%, at least about 85%, at least about 90%, at least about 95% identical to, or is identical to 5′-CAUUAUGGACAGAGUUACCG-3′, or
   wherein the first spacer has a sequence that is at least about 75%, at least about 80%, at least about 85%, at least about 90%, at least about 95% identical to, or is identical to 5′-UGGACCACAUGGCUUUGCUC-3′, and the second spacer has a sequence that is at least about 75%, at least about 80%, at least about 85%, at least about 90%, at least about 95% identical to, or is identical to 5′-ACGUACUCCACCACUGUCAC-3′.
5A. The pharmaceutical composition of clause 1A,
   wherein the first spacer has a sequence comprising nucleotides 6 to 20, 5 to 20, 4 to 20, 3 to 20, 2 to 20, or 1 to 20 of the following sequence 5′-CUGUCACCAGGCAUUGUGUC-3′, and the second spacer has a sequence comprising nucleotides 6 to 20, 5 to 20, 4 to 20, 3 to 20, 2 to 20, or 1 to 20 of the following sequence 5′-UGUCCUUGCAACUCUCACGG-3′,
   wherein the first spacer has a sequence comprising nucleotides 6 to 20, 5 to 20, 4 to 20, 3 to 20, 2 to 20, or 1 to 20 of the following sequence 5′-GUAGUAGCAGUCCUGUACCC-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
   wherein the first spacer has a sequence comprising nucleotides 6 to 20, 5 to 20, 4 to 20, 3 to 20, 2 to 20, or 1 to 20 of the following sequence 5′-AGGACACUCGAUUCUGUCA-3′, and the second spacer has a sequence comprising nucleotides 6 to 20, 5 to 20, 4 to 20, 3 to 20, 2 to 20, or 1 to 20 of the following sequence 5′-CACAACUCCCACAGUGGCCC-3′,
   wherein the first spacer has a sequence comprising nucleotides 6 to 20, 5 to 20, 4 to 20, 3 to 20, 2 to 20, or 1 to 20 of the following sequence 5′-CUGUCACUGGACAUUGUGUC-3′, and the second spacer has a sequence comprising nucleotides 6 to 20, 5 to 20, 4 to 20, 3 to 20, 2 to 20, or 1 to 20 of the following sequence 5′-AAGUGUCCUUGCGACGUCCA-3′,
   wherein the first spacer has a sequence comprising nucleotides 6 to 20, 5 to 20, 4 to 20, 3 to 20, 2 to 20, or 1 to 20 of the following sequence 5′-GGAGCAAAGCCCCACAGUCC-3′, and the second spacer has a sequence comprising nucleotides 6 to 20, 5 to 20, 4 to 20, 3 to 20, 2 to 20, or 1 to 20 of the following sequence 5′-GUUGGUGCUGAAAUUCAAAG-3′,
   wherein the first spacer has a sequence comprising nucleotides 6 to 20, 5 to 20, 4 to 20, 3 to 20, 2 to 20, or 1 to 20 of the following sequence 5′-GGAGCAAAGCCCCGGGGUCC-3′, and the second spacer has a sequence comprising nucleotides 6 to 20, 5 to 20, 4 to 20, 3 to 20, 2 to 20, or 1 to 20 of the following sequence 5′-GUUGGUGCUGAAAUUCAAAG-3′,
   wherein the first spacer has a sequence comprising nucleotides 6 to 20, 5 to 20, 4 to 20, 3 to 20, 2 to 20, or 1 to 20 of the following sequence 5′-CUGGAACUGGGACCACCGU-3′, and the second spacer has a sequence comprising nucleotides 6 to 20, 5 to 20, 4 to 20, 3 to 20, 2 to 20, or 1 to 20 of the following sequence 5′-ACAGAGCUUCCUUCUGAAGA-3′,
   wherein the first spacer has a sequence comprising nucleotides 6 to 20, 5 to 20, 4 to 20, 3 to 20, 2 to 20, or 1 to 20 of the following sequence 5′-AUGCCAGUGUGGUGUCAUAG-3′, and the second spacer has a sequence comprising nucleotides 6 to 20, 5 to 20, 4 to 20, 3 to 20, 2 to 20, or 1 to 20 of the following sequence 5′-ACCACAGAAUACUACCCAAA-3′,
   wherein the first has a spacer sequence comprising nucleotides 6 to 20, 5 to 20, 4 to 20, 3 to 20, 2 to 20, or 1 to 20 of the following sequence 5′-GGAGCCAGAAUAACAUUCGG-3′, and the second spacer has a sequence comprising nucleotides 6 to 20, 5 to 20, 4 to 20, 3 to 20, 2 to 20, or 1 to 20 of the following sequence 5′-CUAGAGGCUUUUUUUGAACA-3′;
   wherein the first spacer has a sequence comprising nucleotides 6 to 20, 5 to 20, 4 to 20, 3 to 20, 2 to 20, or 1 to 20 of the following sequence 5′-CAGAUGCUGAGAUUAGUCCU-3′, and the second spacer has a sequence comprising nucleotides 6 to 20, 5 to 20, 4 to 20, 3 to 20, 2 to 20, or 1 to 20 of the following sequence 5′-UGGAUUCCUGCAGUAGUUCC-3′,
   wherein the first spacer has a sequence comprising nucleotides 6 to 20, 5 to 20, 4 to 20, 3 to 20, 2 to 20, or 1 to 20 of the following sequence 5′-UGACACCACAUUGGCAUCGG-3′, and the second spacer has a sequence comprising nucleotides 6 to 20, 5 to 20, 4 to 20, 3 to 20, 2 to 20, or 1 to 20 of the following sequence 5′-ACAUGUUCUUCCUGUGAUAG-3′,
   wherein the first spacer has a sequence comprising nucleotides 6 to 20, 5 to 20, 4 to 20, 3 to 20, 2 to 20, or 1 to 20 of the following sequence 5′-CAUAGAUGACCAAGAUUGAC-3′, and the second spacer has a sequence comprising nucleotides 6 to 20, 5 to 20, 4 to 20, 3 to 20, 2 to 20, or 1 to 20 of the following sequence 5′-UGAUACCACACUGGCAUCAG-3′,
   wherein the first spacer has a sequence comprising nucleotides 6 to 20, 5 to 20, 4 to 20, 3 to 20, 2 to 20, or 1 to 20 of the following sequence 5′-CCAUCACUGGACAUUGCGUC-3′, and the second spacer has a sequence comprising nucleotides 6 to 20, 5 to 20, 4 to 20, 3 to 20, 2 to 20, or 1 to 20 of the following sequence 5′-AACUCUCCUCACAACUCCCA-3′,
   wherein the first spacer has a sequence comprising nucleotides 6 to 20, 5 to 20, 4 to 20, 3 to 20, 2 to 20, or 1 to 20 of the following sequence 5′-CUGCAUCUGAGCAUCGUGUC-3′, and the second spacer has a sequence comprising nucleotides 6 to 20, 5 to 20, 4 to 20, 3 to 20, 2 to 20, or 1 to 20 of the following sequence 5′-CGUCCCUCCGAAUGUUAUUC-3′,
   wherein the first spacer has a sequence comprising nucleotides 6 to 20, 5 to 20, 4 to 20, 3 to 20, 2 to 20, or 1 to 20 of the following sequence 5′-AAACAGCCGUGGACGUCGCA-3′, and the second spacer has a sequence comprising nucleotides 6 to 20, 5 to 20, 4 to 20, 3 to 20, 2 to 20, or 1 to 20 of the following sequence 5′-UGAACAAGGUAAGAAGUCUC-3′,
   wherein the first spacer has a sequence comprising nucleotides 6 to 20, 5 to 20, 4 to 20, 3 to 20, 2 to 20, or 1 to 20 of the following sequence 5′-ACAGAGGCUCCUUCUGAACA-3′, and the second spacer has a sequence comprising nucleotides 6 to 20, 5 to 20, 4 to 20, 3 to 20, 2 to 20, or 1 to 20 of the following sequence 5′-GCUUGGAACCGGGGCCACUG-3′,
   wherein the first spacer has a sequence comprising nucleotides 6 to 20, 5 to 20, 4 to 20, 3 to 20, 2 to 20, or 1 to 20 of the following sequence 5′-AUGCCAGUGUGGUGUCAUAG-3′, and the second spacer has a sequence comprising nucleotides 6 to 20, 5 to 20, 4 to 20, 3 to 20, 2 to 20, or 1 to 20 of the following sequence 5′-ACAACAGAAUAUUAUCCAAA-3′,
   wherein the first spacer has a sequence comprising nucleotides 6 to 20, 5 to 20, 4 to 20, 3 to 20, 2 to 20, or 1 to 20 of the following sequence 5′-CUAUGACACCACAUUGGCAU-3′, and the second spacer has a sequence comprising nucleotides 6 to 20, 5 to 20, 4 to 20, 3 to 20, 2 to 20, or 1 to 20 of the following sequence 5′-ACAUGUUCUUCCUGUGAUAG-3′,
   wherein the first spacer has a sequence comprising nucleotides 6 to 20, 5 to 20, 4 to 20, 3 to 20, 2 to 20, or 1 to 20 of the following sequence 5′-AAUAACAUUCGGAGGGACGA-3′, and the second spacer has a sequence comprising nucleotides 6 to 20, 5 to 20, 4 to 20, 3 to 20, 2 to 20, or 1 to 20 of the following sequence 5′-UAUUCUGGCUCCAAGCCUAG-3′,
   wherein the first spacer has a sequence comprising nucleotides 6 to 20, 5 to 20, 4 to 20, 3 to 20, 2 to 20, or 1 to 20 of the following sequence 5′-GUAGCAGUCCUGUACCCCGG-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
   wherein the first spacer has a sequence comprising nucleotides 6 to 20, 5 to 20, 4 to 20, 3 to 20, 2 to 20, or 1 to 20 of the following sequence 5′-AGUAGCAGUCCUGUACCCCG-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
   wherein the first spacer has a sequence comprising nucleotides 6 to 20, 5 to 20, 4 to 20, 3 to 20, 2 to 20, or 1 to 20 of the following sequence 5′-UAGUAGCAGUCCUGUACCCC-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′, or
   wherein the first spacer has a sequence comprising nucleotides 6 to 20, 5 to 20, 4 to 20, 3 to 20, 2 to 20, or 1 to 20 of the following sequence 5′-UGGACCACAUGGCUUUGCUC-3′, and the second spacer has a sequence comprising nucleotides 6 to 20, 5 to 20, 4 to 20, 3 to 20, 2 to 20, or 1 to 20 of the following sequence 5′-ACGUACUCCACCACUGUCAC-3′.
6A. The pharmaceutical composition of any one of clauses 1A to 5A, wherein the first spacer and/or the second spacer comprise a modified nucleotide.
7A. The pharmaceutical composition of any one of clauses 1A to 6A, wherein the one or more nickases comprise a CRSPR Cas nickase.
8A. The pharmaceutical composition of any one of clause 1A to 7A, wherein at least one of the one or more Cas nickases, when in operative interaction with the first or second guide oligonucleotide, is engineered to nick the opposite strand of the LPA gene to which the operative guide oligonucleotide (gRNA) is hybridized.
9A. The pharmaceutical composition of clause 8A, wherein at least one of the one or more polynucleotides (mRNAs) encoding the one or more Cas nickases encodes a Streptococcus pyogenes Cas9 nickase bearing a H840A mutation.
10A. The pharmaceutical composition of any one of clauses 1A to 7A, wherein at least one of the one or more Cas nickases, when in operative interaction with the first or second guide oligonucleotide (gRNA), is engineered to nick the same strand of the LPA gene to which the operative guide is hybridized.
11A. The pharmaceutical composition of clause 10A, wherein at least one of the one or more polynucleotides (mRNAs) encoding the one or more Cas nickases encodes a Streptococcus pyogenes Cas9 nickase bearing a D10A mutation.
12A. The pharmaceutical composition of any one of clauses 1A to 11A, wherein at least one of the one or more polynucleotides (mRNAs) comprises:
   (a) a 5′ untranslated region (UTR);
   (b) a 3′ UTR region;
   (c) a poly(A) tail proximate to the 3′ UTR as compared to the 5′ UTR, said poly(A) tail comprising a chain of 80-150 nucleotides length that comprise adenine nucleotides; and
   (d) a gene editor coding region encoding an impaired CRISPR Cas9 endonuclease domain and a polymerase domain, said gene editor coding region extending between the 5′ UTR and the 3′ UTR.
13A. The pharmaceutical composition of any one of clauses 1A to 7A, wherein at least one of the one or more polynucleotides is selected from any of the mRNA sequences listed in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the mRNA sequences listed in Table 1.
14A. The pharmaceutical composition of any one of clauses 1A to 7A, wherein at least one of the one or more polynucleotides comprises a coding sequence of any of the mRNA sequences listed in Table 1 or comprises a coding sequence having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the coding sequences of the SpCas9-D10A nickase mRNA sequences listed in Table 1.
15A. The pharmaceutical composition of any one of clauses 1A to 14A, wherein the delivery system comprises lipid nanoparticles (LNPs), wherein the LNPs comprise:
   (a) one or more ionizable lipids,
   (b) cholesterol,
   (c) one or more PEG-lipids,
   (d) a phospholipid; and
   (e) optionally including a targeting moiety, such as a GalNAc lipid.
16A. The pharmaceutical composition of clause 15A, wherein the LNPs are formulated to comprise:
   (a) 40 to 60 molar percent of the one or more ionizable lipids, 28 to 48 molar percent of the cholesterol, 5 to 13 molar percent of the phospholipid, and 2 to 5 molar percent of the PEG-lipid,
   (b) 50+/−10% molar percent of the one or more ionizable lipids, 38+/−10% molar percent of the cholesterol, 9+/−10% molar percent of the phospholipid, and 3+/−10% molar percent of the PEG-lipid,
   (c) 40 to 60 molar percent of the one or more ionizable lipids, 27.95 to 47.95 molar percent of the cholesterol, 5 to 13 molar percent of the phospholipid, 2 to 5 molar percent of the PEG-lipid, and 0.02 to 0.09 molar percent of the GalNAc-lipid; or
   (d) 50+/−10% molar percent of the one or more ionizable lipids, 37.95+/−10% molar percent of the cholesterol, 9+/−10% molar percent of the phospholipid, 3+/−10% molar percent of the PEG-lipid, and 0.05+/−10% molar percent of the GalNAc-lipid.
17A. The pharmaceutical composition of any one of clauses 1A to 16A, wherein the sequences of the first and second scaffold region are the same.
18A. The pharmaceutical composition of any one of clauses 1A to 17A, wherein the scaffold region sequences of the first and second guide oligonucleotides (gRNAs) are each independently selected from one of the following sequences or a sequence having at least about 75%, at least about 80%, at least about 85%, at least about 90%, or at least about 95% identity to one of the following sequences:

5′-GUUUUAGAGCUAGAAAUAGCAAGUUAAAAUAAGGCUAGUCCGUUAUC
AACUUGAAAAAGUGGCACCGAGUCGGUGC-3′,
and
5′-GUUUGAGAGCUAUGCUGGAAACAGCAUAGCAAGUUCAAAUAAGGCUA
GUCCGUUAUCAACUUGAAAAAGUGGCACCGAGUCGGUGC-3′.

19A. The pharmaceutical composition of any one of clauses 1A to 18A, wherein the first scaffold and/or the second scaffold comprise a modified nucleotide.
20A. The pharmaceutical composition of any one or clauses 1A to 19A, wherein the scaffold comprises one or more of the modified nucleotides in the following nucleotide sequence: 5′-mGUUUUAGmAmGmCmUmAGmAmAmAmUmAmGmCmAmAGUUmAAmAAmU AmAmGmGmCmUmAGUmCmCGUUAmUmCAAmCmUmUGmAmAmAmAmAmG mUmGGmCmAmCmCmGmAmGmUmCmGmGmUmGmC 3′, where m of mN is 2′-O-methyl ribose.
21A. The pharmaceutical composition of any one of clauses 1A to 20A, wherein the first guide oligonucleotide and/or the second guide oligonucleotide comprise an RNA motif at the 3′ end of the guide oligonucleotide.
22A. The pharmaceutical composition of clause 21A, wherein the RNA motif comprises, consists essentially of, or consists of 5′-UUU-3′.
23A. The pharmaceutical composition of clause 21A, wherein the RNA motif comprises, consists essentially of, or consists of 5′-*mU*mU*mU-3′, where “mU*” indicates a phosphorothioated 2′-O-methyl uracil base, and “mU” indicates a 2′-O-methyl uracil base.
24A. The pharmaceutical composition according to any one of clauses 1A to 23A, wherein administration of the composition to hepatocytes results in 40% or greater editing efficiency.
25A. The pharmaceutical composition according to clause 24A, wherein the hepatocytes are primary hepatocytes.
26A. The pharmaceutical composition according to clause 24A or 25A, wherein the hepatocytes are human hepatocytes.
27A. A method for inactivating the LPA gene in vivo in a mammalian subject to treat and prevent cardiovascular disease comprising the step of: administering to the subject a pharmaceutical composition of any one of clauses 1A to 26A.
28A. A method for reducing blood Lp(a) concentration in a mammalian subject to treat and prevent cardiovascular disease comprising the step of: administering to the subject a pharmaceutical composition of any one of clauses 1A to 26A.
29A. A method for treating and/or preventing cardiovascular disease associated with the LPA gene in a mammalian subject comprising the step of: administering to the subject a pharmaceutical composition of any one of clauses 1A to 26A.
30A. A method for in vivo editing of an LPA gene in a mammalian subject comprising: administering to the subject a pharmaceutical composition comprising:
   (i) one or more polynucleotides (mRNAs) encoding one or more CRISPR Cas nickases,
   (v) a first guide oligonucleotide (gRNA) comprising a first spacer sequence and a scaffold region; and
   (vi) a second guide oligonucleotide (gRNA) comprising a second spacer sequence and a scaffold region, and
   (vii) a delivery system that is engineered to deliver the one or more mRNAs, the first gRNA, and/or the second gRNA individually or collectively to the liver,
   wherein the first gRNA and at least one of the one or more Cas nickases are engineered to cause the at least one of the one or more Cas nickases to nick one of the first or second strands of the LPA gene at a first location of chromosome 6 from position 160,664,275 to160,531,482, and
   wherein the second gRNA and at least one of the one or more Cas nickases are engineered to cause the at least one of the one or more Cas nickases to nick the other of the first or second strands of the LPA gene at a second location of chromosome 6 from position 160,664,275 tol60,531,482.
31A. A gene editing system for editing the LPA gene produced by expressing in a cell one or more exogenous polynucleotides (mRNA) encoding one or more CRISPR Cas nickases and introducing first and second gRNAs into the cell,
   wherein the first guide oligonucleotide (gRNA) comprises (i) a first spacer sequence that is complementary to a first strand of the LPA gene at a first target sequence and (ii) a first scaffold region that serves as a binding scaffold for at least one of the one or more Cas nickases,
   wherein the second guide oligonucleotide (gRNA) comprises (i) a second spacer sequence that is complementary to a second strand of the LPA gene at a second target sequence and (ii) a second scaffold region that serves as a binding scaffold for at least one of the one or more Cas nickases,
   wherein the first gRNA and the at least one of the one or more Cas nickases are engineered to cause the at least one of the one or more Cas nickases to nick one of the first or second strands of the LPA gene at a first location of chromosome 6 from position 160,664,275 to 160,531,482, and
   wherein the second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.
32A. A gene editing system comprising:
   a means for expressing one or more CRISPR Cas nickases in a cell; and
   a means for directing the one or more Cas nickases to first and second locations in the LPA gene and to cause the one or more Cas nickases to introduce a nick in a first strand of the LPA gene and to introduce a nick in a second strand of the LPA gene.
33A. A pharmaceutical composition comprising the gene editing system of clause 32A and a delivery system.
34A. The pharmaceutical composition of clause 33A, wherein the delivery system comprises means for delivering to the cell the means for expressing one or more CRISPR Cas nickases and the means for directing the one or more Cas nickases to first and second locations in the LPA gene.
35A. A gene editing system for editing the LPA gene comprising:
   a Cas nickase or a nucleic acid encoding the Cas nickase;
   a first guide oligonucleotide comprising (i) a first spacer sequence that is complementary to a first strand of the LPA gene at a first target sequence and (ii) a first scaffold region that serves as a binding scaffold for the Cas nickase;
   a second guide oligonucleotide comprising (i) a second spacer sequence that is complementary to a second strand of the LPA gene at a second target sequence and (ii) a second scaffold region that serves as a binding scaffold for the Cas nickase,
   wherein the first guide oligonucleotide and the Cas nickase are engineered to cause the Cas nickase to nick one of the first or second strands of the LPA gene at a first location,
   wherein the second guide oligonucleotide and the Cas nickase are engineered to cause the Cas nickase to nick the other of the first or second strands of the LPA gene at a second location, and
   wherein the first location and the second location are spaced apart by a distance of 1 to 200 nucleotides.
36A. The gene editing system of clause 35A, wherein the first location and the second location are spaced apart by a distance of 20 to 50 nucleotides.
1B. A pharmaceutical composition for in vivo editing of an LPA gene, in a mammalian subject, comprising:
   (i) an engineered, non-naturally occurring gene editing system, which comprises:
      (a) one or more polynucleotides (mRNAs) encoding a nickase;
      (b) a first guide oligonucleotide (gRNA) comprising a first spacer sequence that includes a region that is complementary to a first strand of the LPA gene at a first target sequence and a first scaffold region that serves as a binding scaffold for the nickase; and
      (c) a second guide oligonucleotide (gRNA) comprising a second spacer sequence that includes a region that is complementary to a second strand of the LPA gene at a second target sequence and a second scaffold region that serves as a binding scaffold for the nickase, and
   (ii) lipid nanoparticles (LNPs) that encapsulate the gene editing system,
   wherein the first and second strands are opposing strands, and
   wherein the first spacer has a sequence that is at least about 75%, at least about 80%, at least about 85%, at least about 90%, at least about 95% similar to or identical to, or is identical to the guide 1 protospacer listed in Table 2 or Table 5, and wherein the second spacer has a sequence that is at least about 75%, at least about 80%, at least about 85%, at least about 90%, at least about 95% similar to or identical to, or is identical to the guide 2 protospacer listed in Table 2 or Table 5.
2B. The pharmaceutical composition of clause 1B, wherein the first gRNA and the nickase are engineered to cause the nickase to nick one of the first or second strands of the LPA gene at a first location of chromosome 6 from position 160,664,275 to 160,531,482, and wherein the second gRNA and the nickase are engineered to cause the nickase to nick the other of the first or second strands of the LPA gene at a second location of chromosome 6 from position 160,664,275 to 160,531,482.
3B. The pharmaceutical composition of clause 1B or 2B, wherein the first spacer comprises a sequence identical to or substantially identical to a guide 1 protospacer listed in Table 2 or Table 5, and the second spacer comprises a sequence identical to or substantially identical to a corresponding guide 2 protospacer (in the same row) listed in Table 2 or Table 5.
4B. The pharmaceutical composition of clause 1B,
   wherein the first spacer has a sequence that is at least about 75%, at least about 80%, at least about 85%, at least about 90%, at least about 95% identical to, or is identical to 5′-CUGUCACCAGGCAUUGUGUC-3′, and the second spacer has a sequence that is at least about 75%, at least about 80%, at least about 85%, at least about 90%, at least about 95% identical to, or is identical to 5′-UGUCCUUGCAACUCUCACGG-3′,
   wherein the first spacer has a sequence that is at least about 75%, at least about 80%, at least about 85%, at least about 90%, at least about 95% identical to, or is identical to 5′-GUAGUAGCAGUCCUGUACCC-3′, and the second spacer has a sequence that is at least about 75%, at least about 80%, at least about 85%, at least about 90%, at least about 95% identical to, or is identical to 5′-CAUUAUGGACAGAGUUACCG-3′,
   wherein the first spacer has a sequence that is at least about 75%, at least about 80%, at least about 85%, at least about 90%, at least about 95% identical to, or is identical to 5′-AGGACACUCGAUUCUGUCA-3′, and the second spacer has a sequence that is at least about 75%, at least about 80%, at least about 85%, at least about 90%, at least about 95% identical to, or is identical to 5′-CACAACUCCCACAGUGGCCC-3′,
   wherein the first spacer has a sequence that is at least about 75%, at least about 80%, at least about 85%, at least about 90%, at least about 95% identical to, or is identical to 5′-CUGUCACUGGACAUUGUGUC-3′, and the second spacer has a sequence that is at least about 75%, at least about 80%, at least about 85%, at least about 90%, at least about 95% identical to, or is identical to 5′-AAGUGUCCUUGCGACGUCCA-3′,
   wherein the first spacer has a sequence that is at least about 75%, at least about 80%, at least about 85%, at least about 90%, at least about 95% identical to, or is identical to 5′-GGAGCAAAGCCCCACAGUCC-3′, and the second spacer has a sequence that is at least about 75%, at least about 80%, at least about 85%, at least about 90%, at least about 95% identical to, or is identical to 5′-GUUGGUGCUGAAAUUCAAAG-3′,
   wherein the first spacer has a sequence that is at least about 75%, at least about 80%, at least about 85%, at least about 90%, at least about 95% identical to, or is identical to 5′-GGAGCAAAGCCCCGGGGUCC-3′, and the second spacer has a sequence that is at least about 75%, at least about 80%, at least about 85%, at least about 90%, at least about 95% identical to, or is identical to 5′-GUUGGUGCUGAAAUUCAAAG-3′,
   wherein the first spacer has a sequence that is at least about 75%, at least about 80%, at least about 85%, at least about 90%, at least about 95% identical to, or is identical to 5′-CUGGAACUGGGACCACCGU-3′, and the second spacer has a sequence that is at least about 75%, at least about 80%, at least about 85%, at least about 90%, at least about 95% identical to, or is identical to 5′-ACAGAGCUUCCUUCUGAAGA-3′,
   wherein the first spacer has a sequence that is at least about 75%, at least about 80%, at least about 85%, at least about 90%, at least about 95% identical to, or is identical to 5′-AUGCCAGUGUGGUGUCAUAG-3′, and the second spacer has a sequence that is at least about 75%, at least about 80%, at least about 85%, at least about 90%, at least about 95% identical to, or is identical to 5′-ACCACAGAAUACUACCCAAA-3′,
   wherein the first spacer has a sequence that is at least about 75%, at least about 80%, at least about 85%, at least about 90%, at least about 95% identical to, or is identical to 5′-GGAGCCAGAAUAACAUUCGG-3′, and the second spacer has a sequence that is at least about 75%, at least about 80%, at least about 85%, at least about 90%, at least about 95% identical to, or is identical to 5′-CUAGAGGCUUUUUUUGAACA-3′;
   wherein the first spacer has a sequence that is at least about 75%, at least about 80%, at least about 85%, at least about 90%, at least about 95% identical to, or is identical to 5′-CAGAUGCUGAGAUUAGUCCU-3′, and the second spacer has a sequence that is at least about 75%, at least about 80%, at least about 85%, at least about 90%, at least about 95% identical to, or is identical to 5′-UGGAUUCCUGCAGUAGUUCC-3′,
   wherein the first spacer has a sequence that is at least about 75%, at least about 80%, at least about 85%, at least about 90%, at least about 95% identical to, or is identical to 5′-UGACACCACAUUGGCAUCGG-3′, and the second spacer has a sequence that is at least about 75%, at least about 80%, at least about 85%, at least about 90%, at least about 95% identical to, or is identical to 5′-ACAUGUUCUUCCUGUGAUAG-3′,
   wherein the first spacer has a sequence that is at least about 75%, at least about 80%, at least about 85%, at least about 90%, at least about 95% identical to, or is identical to 5′-CAUAGAUGACCAAGAUUGAC-3′, and the second spacer has a sequence that is at least about 75%, at least about 80%, at least about 85%, at least about 90%, at least about 95% identical to, or is identical to 5′-UGAUACCACACUGGCAUCAG-3′,
   wherein the first spacer has a sequence that is at least about 75%, at least about 80%, at least about 85%, at least about 90%, at least about 95% identical to, or is identical to 5′-CCAUCACUGGACAUUGCGUC-3′, and the second spacer has a sequence that is at least about 75%, at least about 80%, at least about 85%, at least about 90%, at least about 95% identical to, or is identical to 5′-AACUCUCCUCACAACUCCCA-3′,
   wherein the first spacer has a sequence that is at least about 75%, at least about 80%, at least about 85%, at least about 90%, at least about 95% identical to, or is identical to 5′-CUGCAUCUGAGCAUCGUGUC-3′, and the second spacer has a sequence that is at least about 75%, at least about 80%, at least about 85%, at least about 90%, at least about 95% identical to, or is identical to 5′-CGUCCCUCCGAAUGUUAUUC-3′,
   wherein the first spacer has a sequence that is at least about 75%, at least about 80%, at least about 85%, at least about 90%, at least about 95% identical to, or is identical to 5′-AAACAGCCGUGGACGUCGCA-3′, and the second spacer has a sequence that is at least about 75%, at least about 80%, at least about 85%, at least about 90%, at least about 95% identical to, or is identical to 5′-UGAACAAGGUAAGAAGUCUC-3′,
   wherein the first spacer has a sequence that is at least about 75%, at least about 80%, at least about 85%, at least about 90%, at least about 95% identical to, or is identical to 5′-ACAGAGGCUCCUUCUGAACA-3′, and the second spacer has a sequence that is at least about 75%, at least about 80%, at least about 85%, at least about 90%, at least about 95% identical to, or is identical to 5′-GCUUGGAACCGGGGCCACUG-3′,
   wherein the first spacer has a sequence that is at least about 75%, at least about 80%, at least about 85%, at least about 90%, at least about 95% identical to, or is identical to 5′-AUGCCAGUGUGGUGUCAUAG-3′, and the second spacer has a sequence that is at least about 75%, at least about 80%, at least about 85%, at least about 90%, at least about 95% identical to, or is identical to 5′-ACAACAGAAUAUUAUCCAAA-3′,
   wherein the first spacer has a sequence that is at least about 75%, at least about 80%, at least about 85%, at least about 90%, at least about 95% identical to, or is identical to 5′-CUAUGACACCACAUUGGCAU-3′, and the second spacer has a sequence that is at least about 75%, at least about 80%, at least about 85%, at least about 90%, at least about 95% identical to, or is identical to 5′-ACAUGUUCUUCCUGUGAUAG-3′,
   wherein the first spacer has a sequence that is at least about 75%, at least about 80%, at least about 85%, at least about 90%, at least about 95% identical to, or is identical to 5′-AAUAACAUUCGGAGGGACGA-3′, and the second spacer has a sequence that is at least about 75%, at least about 80%, at least about 85%, at least about 90%, at least about 95% identical to, or is identical to 5′-UAUUCUGGCUCCAAGCCUAG-3′,
   wherein the first spacer has a sequence that is at least about 75%, at least about 80%, at least about 85%, at least about 90%, at least about 95% identical to, or is identical to 5′-GUAGCAGUCCUGUACCCCGG-3′, and the second spacer has a sequence that is at least about 75%, at least about 80%, at least about 85%, at least about 90%, at least about 95% identical to, or is identical to 5′-CAUUAUGGACAGAGUUACCG-3′,
   wherein the first spacer has a sequence that is at least about 75%, at least about 80%, at least about 85%, at least about 90%, at least about 95% identical to, or is identical to 5′-AGUAGCAGUCCUGUACCCCG-3′, and the second spacer has a sequence that is at least about 75%, at least about 80%, at least about 85%, at least about 90%, at least about 95% identical to, or is identical to 5′-CAUUAUGGACAGAGUUACCG-3′,
   wherein the first spacer has a sequence that is at least about 75%, at least about 80%, at least about 85%, at least about 90%, at least about 95% identical to, or is identical to 5′-UAGUAGCAGUCCUGUACCCC-3′, and the second spacer has a sequence that is at least about 75%, at least about 80%, at least about 85%, at least about 90%, at least about 95% identical to, or is identical to 5′-CAUUAUGGACAGAGUUACCG-3′, or
   wherein the first spacer has a sequence that is at least about 75%, at least about 80%, at least about 85%, at least about 90%, at least about 95% identical to, or is identical to 5′-UGGACCACAUGGCUUUGCUC-3′, and the second spacer has a sequence that is at least about 75%, at least about 80%, at least about 85%, at least about 90%, at least about 95% identical to, or is identical to 5′-ACGUACUCCACCACUGUCAC-3′.
5B. The pharmaceutical composition of clause 1B,
   wherein the first spacer has a sequence comprising nucleotides 6 to 20, 5 to 20, 4 to 20, 3 to 20, 2 to 20, or 1 to 20 of the following sequence 5′-CUGUCACCAGGCAUUGUGUC-3′, and the second spacer has a sequence comprising nucleotides 6 to 20, 5 to 20, 4 to 20, 3 to 20, 2 to 20, or 1 to 20 of the following sequence 5′-UGUCCUUGCAACUCUCACGG-3′,
   wherein the first spacer has a sequence comprising nucleotides 6 to 20, 5 to 20, 4 to 20, 3 to 20, 2 to 20, or 1 to 20 of the following sequence 5′-GUAGUAGCAGUCCUGUACCC-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
   wherein the first spacer has a sequence comprising nucleotides 6 to 20, 5 to 20, 4 to 20, 3 to 20, 2 to 20, or 1 to 20 of the following sequence 5′-AGGACACUCGAUUCUGUCA-3′, and the second spacer has a sequence comprising nucleotides 6 to 20, 5 to 20, 4 to 20, 3 to 20, 2 to 20, or 1 to 20 of the following sequence 5′-CACAACUCCCACAGUGGCCC-3′,
   wherein the first spacer has a sequence comprising nucleotides 6 to 20, 5 to 20, 4 to 20, 3 to 20, 2 to 20, or 1 to 20 of the following sequence 5′-CUGUCACUGGACAUUGUGUC-3′, and the second spacer has a sequence comprising nucleotides 6 to 20, 5 to 20, 4 to 20, 3 to 20, 2 to 20, or 1 to 20 of the following sequence 5′-AAGUGUCCUUGCGACGUCCA-3′,
   wherein the first spacer has a sequence comprising nucleotides 6 to 20, 5 to 20, 4 to 20, 3 to 20, 2 to 20, or 1 to 20 of the following sequence 5′-GGAGCAAAGCCCCACAGUCC-3′, and the second spacer has a sequence comprising nucleotides 6 to 20, 5 to 20, 4 to 20, 3 to 20, 2 to 20, or 1 to 20 of the following sequence 5′-GUUGGUGCUGAAAUUCAAAG-3′,
   wherein the first spacer has a sequence comprising nucleotides 6 to 20, 5 to 20, 4 to 20, 3 to 20, 2 to 20, or 1 to 20 of the following sequence 5′-GGAGCAAAGCCCCGGGGUCC-3′, and the second spacer has a sequence comprising nucleotides 6 to 20, 5 to 20, 4 to 20, 3 to 20, 2 to 20, or 1 to 20 of the following sequence 5′-GUUGGUGCUGAAAUUCAAAG-3′,
   wherein the first spacer has a sequence comprising nucleotides 6 to 20, 5 to 20, 4 to 20, 3 to 20, 2 to 20, or 1 to 20 of the following sequence 5′-CUGGAACUGGGACCACCGU-3′, and the second spacer has a sequence comprising nucleotides 6 to 20, 5 to 20, 4 to 20, 3 to 20, 2 to 20, or 1 to 20 of the following sequence 5′-ACAGAGCUUCCUUCUGAAGA-3′,
   wherein the first spacer has a sequence comprising nucleotides 6 to 20, 5 to 20, 4 to 20, 3 to 20, 2 to 20, or 1 to 20 of the following sequence 5′-AUGCCAGUGUGGUGUCAUAG-3′, and the second spacer has a sequence comprising nucleotides 6 to 20, 5 to 20, 4 to 20, 3 to 20, 2 to 20, or 1 to 20 of the following sequence 5′-ACCACAGAAUACUACCCAAA-3′,
   wherein the first has a spacer sequence comprising nucleotides 6 to 20, 5 to 20, 4 to 20, 3 to 20, 2 to 20, or 1 to 20 of the following sequence 5′-GGAGCCAGAAUAACAUUCGG-3′, and the second spacer has a sequence comprising nucleotides 6 to 20, 5 to 20, 4 to 20, 3 to 20, 2 to 20, or 1 to 20 of the following sequence 5′-CUAGAGGCUUUUUUUGAACA-3′;
   wherein the first spacer has a sequence comprising nucleotides 6 to 20, 5 to 20, 4 to 20, 3 to 20, 2 to 20, or 1 to 20 of the following sequence 5′-CAGAUGCUGAGAUUAGUCCU-3′, and the second spacer has a sequence comprising nucleotides 6 to 20, 5 to 20, 4 to 20, 3 to 20, 2 to 20, or 1 to 20 of the following sequence 5′-UGGAUUCCUGCAGUAGUUCC-3′,
   wherein the first spacer has a sequence comprising nucleotides 6 to 20, 5 to 20, 4 to 20, 3 to 20, 2 to 20, or 1 to 20 of the following sequence 5′-UGACACCACAUUGGCAUCGG-3′, and the second spacer has a sequence comprising nucleotides 6 to 20, 5 to 20, 4 to 20, 3 to 20, 2 to 20, or 1 to 20 of the following sequence 5′-ACAUGUUCUUCCUGUGAUAG-3′,
   wherein the first spacer has a sequence comprising nucleotides 6 to 20, 5 to 20, 4 to 20, 3 to 20, 2 to 20, or 1 to 20 of the following sequence 5′-CAUAGAUGACCAAGAUUGAC-3′, and the second spacer has a sequence comprising nucleotides 6 to 20, 5 to 20, 4 to 20, 3 to 20, 2 to 20, or 1 to 20 of the following sequence 5′-UGAUACCACACUGGCAUCAG-3′,
   wherein the first spacer has a sequence comprising nucleotides 6 to 20, 5 to 20, 4 to 20, 3 to 20, 2 to 20, or 1 to 20 of the following sequence 5′-CCAUCACUGGACAUUGCGUC-3′, and the second spacer has a sequence comprising nucleotides 6 to 20, 5 to 20, 4 to 20, 3 to 20, 2 to 20, or 1 to 20 of the following sequence 5′-AACUCUCCUCACAACUCCCA-3′,
   wherein the first spacer has a sequence comprising nucleotides 6 to 20, 5 to 20, 4 to 20, 3 to 20, 2 to 20, or 1 to 20 of the following sequence 5′-CUGCAUCUGAGCAUCGUGUC-3′, and the second spacer has a sequence comprising nucleotides 6 to 20, 5 to 20, 4 to 20, 3 to 20, 2 to 20, or 1 to 20 of the following sequence 5′-CGUCCCUCCGAAUGUUAUUC-3′,
   wherein the first spacer has a sequence comprising nucleotides 6 to 20, 5 to 20, 4 to 20, 3 to 20, 2 to 20, or 1 to 20 of the following sequence 5′-AAACAGCCGUGGACGUCGCA-3′, and the second spacer has a sequence comprising nucleotides 6 to 20, 5 to 20, 4 to 20, 3 to 20, 2 to 20, or 1 to 20 of the following sequence 5′-UGAACAAGGUAAGAAGUCUC-3′,
   wherein the first spacer has a sequence comprising nucleotides 6 to 20, 5 to 20, 4 to 20, 3 to 20, 2 to 20, or 1 to 20 of the following sequence 5′-ACAGAGGCUCCUUCUGAACA-3′, and the second spacer has a sequence comprising nucleotides 6 to 20, 5 to 20, 4 to 20, 3 to 20, 2 to 20, or 1 to 20 of the following sequence 5′-GCUUGGAACCGGGGCCACUG-3′,
   wherein the first spacer has a sequence comprising nucleotides 6 to 20, 5 to 20, 4 to 20, 3 to 20, 2 to 20, or 1 to 20 of the following sequence 5′-AUGCCAGUGUGGUGUCAUAG-3′, and the second spacer has a sequence comprising nucleotides 6 to 20, 5 to 20, 4 to 20, 3 to 20, 2 to 20, or 1 to 20 of the following sequence 5′-ACAACAGAAUAUUAUCCAAA-3′,
   wherein the first spacer has a sequence comprising nucleotides 6 to 20, 5 to 20, 4 to 20, 3 to 20, 2 to 20, or 1 to 20 of the following sequence 5′-CUAUGACACCACAUUGGCAU-3′, and the second spacer has a sequence comprising nucleotides 6 to 20, 5 to 20, 4 to 20, 3 to 20, 2 to 20, or 1 to 20 of the following sequence 5′-ACAUGUUCUUCCUGUGAUAG-3′,
   wherein the first spacer has a sequence comprising nucleotides 6 to 20, 5 to 20, 4 to 20, 3 to 20, 2 to 20, or 1 to 20 of the following sequence 5′-AAUAACAUUCGGAGGGACGA-3′, and the second spacer has a sequence comprising nucleotides 6 to 20, 5 to 20, 4 to 20, 3 to 20, 2 to 20, or 1 to 20 of the following sequence 5′-UAUUCUGGCUCCAAGCCUAG-3′,
   wherein the first spacer has a sequence comprising nucleotides 6 to 20, 5 to 20, 4 to 20, 3 to 20, 2 to 20, or 1 to 20 of the following sequence 5′-GUAGCAGUCCUGUACCCCGG-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
   wherein the first spacer has a sequence comprising nucleotides 6 to 20, 5 to 20, 4 to 20, 3 to 20, 2 to 20, or 1 to 20 of the following sequence 5′-AGUAGCAGUCCUGUACCCCG-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
   wherein the first spacer has a sequence comprising nucleotides 6 to 20, 5 to 20, 4 to 20, 3 to 20, 2 to 20, or 1 to 20 of the following sequence 5′-UAGUAGCAGUCCUGUACCCC-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′, or
   wherein the first spacer has a sequence comprising nucleotides 6 to 20, 5 to 20, 4 to 20, 3 to 20, 2 to 20, or 1 to 20 of the following sequence 5′-UGGACCACAUGGCUUUGCUC-3′, and the second spacer has a sequence comprising nucleotides 6 to 20, 5 to 20, 4 to 20, 3 to 20, 2 to 20, or 1 to 20 of the following sequence 5′-ACGUACUCCACCACUGUCAC-3′.
6B. The pharmaceutical composition of any one of clauses 1B to 5B, wherein the first spacer and/or the second spacer comprise a modified nucleotide.
7B. The pharmaceutical composition of clause 6B, wherein one or more nucleotide within five nucleotides from the 5′ end of the first spacer and/or the second spacer are modified nucleotides.
8B. The pharmaceutical composition of clause 6B or 7B, wherein each of the nucleotides within three nucleotides of the 5′ end of the first spacer and/or the second spacer are modified nucleotides.
9B. The pharmaceutical composition of any one of clauses 6B to 8B, wherein the modified nucleotide comprises a 2′-OMe modification and/or a phosphorothioate group.
10B. The pharmaceutical composition of any one of clauses 1B to 9B, wherein the one or more nickases comprise a CRSPR Cas nickase.
11B. The pharmaceutical composition of any one of clauses 1B to 10B, wherein at least one of the one or more Cas nickases, when in operative interaction with the first or second guide oligonucleotide, is engineered to nick the opposite strand of the LPA gene to which the operative guide oligonucleotide (gRNA) is hybridized.
12B. The pharmaceutical composition of clause 11B, wherein at least one of the one or more polynucleotides (mRNAs) encoding the one or more Cas nickases encodes a Streptococcus pyogenes Cas9 nickase bearing a H840A mutation.
13B. The pharmaceutical composition of any one of clauses 1B to 10B, wherein at least one of the one or more Cas nickases, when in operative interaction with the first or second guide oligonucleotide (gRNA), is engineered to nick the same strand of the LPA gene to which the operative guide is hybridized.
14B. The pharmaceutical composition of clauses 13B, wherein at least one of the one or more polynucleotides (mRNAs) encoding the one or more Cas nickases encodes a Streptococcus pyogenes Cas9 nickase bearing a D10A mutation.
15B. The pharmaceutical composition of any one of clauses 1B to 14B, wherein at least one of the one or more polynucleotides (mRNAs) comprises:
   (a) a 5′ untranslated region (UTR);
   (b) a 3′ UTR region;
   (c) a poly(A) tail proximate to the 3′ UTR as compared to the 5′ UTR, said poly(A) tail comprising a chain of 80-150 nucleotides length that comprise adenine nucleotides; and
   (d) a gene editor coding region encoding an impaired CRISPR Cas9 endonuclease domain and a polymerase domain, said gene editor coding region extending between the 5′ UTR and the 3′ UTR.
16B. The pharmaceutical composition of any one of clauses 1B to 15B, wherein at least one of the one or more polynucleotides is selected from any of the mRNA sequences listed in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the mRNA sequences listed in Table 1.
17B. The pharmaceutical composition of any one of clauses 1B to 15B, wherein at least one of the one or more polynucleotides comprises a coding sequence of any of the mRNA sequences listed in Table 1 or comprises a coding sequence having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the coding sequences of the nickase mRNA sequences listed in Table 1.
18B. The pharmaceutical composition of any one of clauses 1B to 17B, wherein the LNPs comprise:
   (a) one or more ionizable lipids,
   (b) cholesterol,
   (c) one or more PEG-lipids,
   (d) a phospholipid; and
   (e) optionally including a targeting moiety, such as a GalNAc lipid.
19B. The pharmaceutical composition of clause 18B, wherein the LNPs are formulated to comprise:
   (a) 40 to 60 molar percent of the one or more ionizable lipids, 28 to 48 molar percent of the cholesterol, 5 to 13 molar percent of the phospholipid, and 2 to 5 molar percent of the PEG-lipid,
   (b) 50+/−10% molar percent of the one or more ionizable lipids, 38+/−10% molar percent of the cholesterol, 9+/−10% molar percent of the phospholipid, and 3+/−10% molar percent of the PEG-lipid,
   (c) 40 to 60 molar percent of the one or more ionizable lipids, 27.95 to 47.95 molar percent of the cholesterol, 5 to 13 molar percent of the phospholipid, 2 to 5 molar percent of the PEG-lipid, and 0.02 to 0.09 molar percent of the GalNAc-lipid; or
   (d) 50+/−10% molar percent of the one or more ionizable lipids, 37.95+/−10% molar percent of the cholesterol, 9+/−10% molar percent of the phospholipid, 3+/−10% molar percent of the PEG-lipid, and 0.05+/−10% molar percent of the GalNAc-lipid.
20B. The pharmaceutical composition of any one of clauses 1B to 19B, wherein the sequences of the first and second scaffold region are the same.
21B. The pharmaceutical composition of any one of clauses 1B to 20B, wherein the scaffold region sequences of the first and second guide oligonucleotides (gRNAs) are each independently selected from one of the following sequences or a sequence having at least about 75%, at least about 80%, at least about 85%, at least about 90%, or at least about 95% similarity or identity to one of the following sequences:

5′-GUUUUAGAGCUAGAAAUAGCAAGUUAAAAUAAGGCUAGUCCGUUAUC
AACUUGAAAAAGUGGCACCGAGUCGGUGC-3′,
and
5′-GUUUGAGAGCUAUGCUGGAAACAGCAUAGCAAGUUCAAAUAAGGCUA
GUCCGUUAUCAACUUGAAAAAGUGGCACCGAGUCGGUGC-3′.

22B. The pharmaceutical composition of any one of clauses 1B to 21B, wherein the first scaffold and/or the second scaffold comprise a modified nucleotide.
23B. The pharmaceutical composition of any one or clauses 1B to 22B, wherein the scaffold comprises one or more of the modified nucleotides in the following nucleotide sequence: 5′-mGUUUUAGmAmGmCmUmAGmAmAmAmUmAmGmCmAmAGUUmAAmAAmU AmAmGmGmCmUmAGUmCmCGUUAmUmCAAmCmUmUGmAmAmAmAmAmG mUmGGmCmAmCmCmGmAmGmUmCmGmGmUmGmC 3′, where m of mN is 2′-O-methyl ribose.
24B. The pharmaceutical composition of any one or clauses 1B to 23B, wherein the scaffold comprises the following sequence or a sequence having at least about 75%, at least about 80%, at least about 85%, at least about 90%, or at least about 95% identity to following sequence: 5′-mGUUUUAGmAmGmCmUmAGmAmAmAmUmAmGmCmAmAGUUmAAmAAmU AmAmGmGmCmUmAGUmCmCGUUAmUmCAAmCmUmUGmAmAmAmAmAmG mUmGGmCmAmCmCmGmAmGmUmCmGmGmUmGmC 3′, where m of mN is 2′-O-methyl ribose.
25B. The pharmaceutical composition of any one of clauses 1B to 24B, wherein the first guide oligonucleotide and/or the second guide oligonucleotide comprise an RNA motif at the 3′ end of the guide oligonucleotide.
26B. The pharmaceutical composition of clause 25B, wherein the RNA motif comprises, consists essentially of, or consists of 5′-UUU-3′.
27B. The pharmaceutical composition of clause 25B, wherein the RNA motif comprises, consists essentially of, or consists of 5′-*mU*mU*mU-3′, where “mU*” indicates a phosphorothioated 2′-O-methyl uracil base, and “mU” indicates a 2′-O-methyl uracil base.
28B. The pharmaceutical composition of any one of clauses 1B to 27B, wherein the weight ratio of the total weight of the first and second guide oligonucleotides to the weight of the mRNA is 1:1+/−25%.
29B. The pharmaceutical composition of any one of clauses 1B to 27B, wherein the weight ratio of the first guide oligonucleotide to the second guide oligonucleotide is 1:1+/−25%.
30B. The gene editing system of clause 2B or any one of clauses 3B to 29B, as they depend from clause 2B, wherein the first location and the second location are spaced apart by less than 200 nucleotides.
31B. The gene editing system of clause 30B, wherein the first location and the second location are spaced apart by a distance of 20 to 50 nucleotides.
32B. The pharmaceutical composition according to any one of clauses 1B to 31B, wherein administration of the composition to hepatocytes results in 40% or greater editing efficiency.
33B. The pharmaceutical composition according to clause 32B, wherein the hepatocytes are primary hepatocytes.
34B. The pharmaceutical composition according to clause 32B or 33B, wherein the hepatocytes are human hepatocytes.
35B. A method for inactivating the LPA gene in vivo in a mammalian subject to treat and prevent cardiovascular disease comprising the step of: administering to the subject a pharmaceutical composition of any one of clauses 1B to 34B.
36B. A method for reducing blood Lp(a) concentration in a mammalian subject to treat and prevent cardiovascular disease comprising the step of: administering to the subject a pharmaceutical composition of any one of clauses 1B to 34B.
37B. A method for treating and/or preventing cardiovascular disease associated with the LPA gene in a mammalian subject comprising the step of: administering to the subject a pharmaceutical composition of any one of clauses 1B to 34B.
38B. A method for in vivo editing of an LPA gene in a mammalian subject comprising the step of: administering to the subject a pharmaceutical composition of any one of clauses 1B to 34B.
39B. A method for in vivo editing of an LPA gene in a mammalian subject comprising: administering to the subject a pharmaceutical composition comprising:
   (i) one or more polynucleotides (mRNAs) encoding one or more CRISPR Cas nickases,
   (ii) a first guide oligonucleotide (gRNA) comprising a first spacer sequence and a scaffold region; and
   (iii) a second guide oligonucleotide (gRNA) comprising a second spacer sequence and a scaffold region, and
   (iv) a delivery system that is engineered to deliver the one or more mRNAs, the first gRNA, and/or the second gRNA individually or collectively to the liver,
   wherein the first gRNA and at least one of the one or more Cas nickases are engineered to cause the at least one of the one or more Cas nickases to nick one of the first or second strands of the LPA gene at a first location of chromosome 6 from position 160,664,275 tol60,531,482, and
   wherein the second gRNA and at least one of the one or more Cas nickases are engineered to cause the at least one of the one or more Cas nickases to nick the other of the first or second strands of the LPA gene at a second location of chromosome 6 from position 160,664,275 to160,531,482.
40B. A gene editing system for editing the LPA gene produced by expressing in a cell one or more exogenous polynucleotides (mRNA) encoding one or more CRISPR Cas nickases and introducing first and second gRNAs into the cell,
   wherein the first guide oligonucleotide (gRNA) comprises (i) a first spacer sequence that is complementary to a first strand of the LPA gene at a first target sequence and (ii) a first scaffold region that serves as a binding scaffold for at least one of the one or more Cas nickases,
   wherein the second guide oligonucleotide (gRNA) comprises (i) a second spacer sequence that is complementary to a second strand of the LPA gene at a second target sequence and (ii) a second scaffold region that serves as a binding scaffold for at least one of the one or more Cas nickases,
   wherein the first gRNA and the at least one of the one or more Cas nickases are engineered to cause the at least one of the one or more Cas nickases to nick one of the first or second strands of the LPA gene at a first location of chromosome 6 from position 160,664,275 to 160,531,482, and
   wherein the second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.
41B. A gene editing system comprising:
   a means for expressing one or more CRISPR Cas nickases in a cell; and
   a means for directing the one or more Cas nickases to first and second locations in the LPA gene and to cause the one or more Cas nickases to introduce a nick in a first strand of the LPA gene and to introduce a nick in a second strand of the LPA gene.
42B. A pharmaceutical composition comprising the gene editing system of clause 41B and a delivery system.
43B. The pharmaceutical composition of clause 42B, wherein the delivery system comprises means for delivering to the cell the means for expressing one or more CRISPR Cas nickases and the means for directing the one or more Cas nickases to first and second locations in the LPA gene.
44B. A gene editing system for editing the LPA gene comprising:
   a Cas nickase or a nucleic acid encoding the Cas nickase;
   a first guide oligonucleotide comprising (i) a first spacer sequence that is complementary to a first strand of the LPA gene at a first target sequence and (ii) a first scaffold region that serves as a binding scaffold for the Cas nickase;
   a second guide oligonucleotide comprising (i) a second spacer sequence that is complementary to a second strand of the LPA gene at a second target sequence and (ii) a second scaffold region that serves as a binding scaffold for the Cas nickase,
   wherein the first guide oligonucleotide and the Cas nickase are engineered to cause the Cas nickase to nick one of the first or second strands of the LPA gene at a first location,
   wherein the second guide oligonucleotide and the Cas nickase are engineered to cause the Cas nickase to nick the other of the first or second strands of the LPA gene at a second location, and
   wherein the first location and the second location are spaced apart by a distance of 1 to 200 nucleotides.
45B. The gene editing system of clause 44B, wherein the first location and the second location are spaced apart by a distance of 20 to 50 nucleotides.

It will also be appreciated from reviewing the present disclosure, that it is contemplated that the one or more aspects or features presented in one of or a group of related clauses may also be included in other clauses or in combination with the one or more aspects or features in other clauses.

All patent and non-patent references and publications cited herein are expressly incorporated herein by reference in their entirety to the extent that they do not conflict with the disclosure presented herein.

1. A pharmaceutical composition for in vivo editing of an LPA gene, in a mammalian subject, comprising:
(i) an engineered, non-naturally occurring gene editing system, which comprises:
(a) one or more polynucleotides (mRNAs) encoding a nickase;
(b) a first guide oligonucleotide (gRNA) comprising a first spacer sequence that includes a region that is complementary to a first strand of the LPA gene at a first target sequence and a first scaffold region that serves as a binding scaffold for the nickase; and
(c) a second guide oligonucleotide (gRNA) comprising a second spacer sequence that includes a region that is complementary to a second strand of the LPA gene at a second target sequence and a second scaffold region that serves as a binding scaffold for the nickase, and
(ii) lipid nanoparticles (LNPs) that encapsulate the gene editing system,
wherein the first and second strands are opposing strands, and
wherein the first spacer has a sequence that is at least about 75%, at least about 80%, at least about 85%, at least about 90%, at least about 95% similar to or identical to, or is identical to the guide 1 protospacer listed in Table 2 or Table 5, and wherein the second spacer has a sequence that is at least about 75%, at least about 80%, at least about 85%, at least about 90%, at least about 95% similar to or identical to, or is identical to the guide 2 protospacer listed in Table 2 or Table 5.

2. The pharmaceutical composition of claim 1, wherein the first gRNA and the nickase are engineered to cause the nickase to nick one of the first or second strands of the LPA gene at a first location of chromosome 6 from position 160,664,275 to 160,531,482, and wherein the second gRNA and the nickase are engineered to cause the nickase to nick the other of the first or second strands of the LPA gene at a second location of chromosome 6 from position 160,664,275 to 160,531,482.

3. The pharmaceutical composition of claim 1, wherein the first spacer comprises a sequence identical to or substantially identical to a guide 1 protospacer listed in Table 2 or Table 5, and the second spacer comprises a sequence identical to or substantially identical to a corresponding guide 2 protospacer (in the same row) listed in Table 2 or Table 5.

4. The pharmaceutical composition of claim 1,
wherein the first spacer has a sequence that is at least about 75%, at least about 80%, at least about 85%, at least about 90%, at least about 95% identical to, or is identical to 5′-CUGUCACCAGGCAUUGUGUC-3′, and the second spacer has a sequence that is at least about 75%, at least about 80%, at least about 85%, at least about 90%, at least about 95% identical to, or is identical to 5′-UGUCCUUGCAACUCUCACGG-3′,
wherein the first spacer has a sequence that is at least about 75%, at least about 80%, at least about 85%, at least about 90%, at least about 95% identical to, or is identical to 5′-GUAGUAGCAGUCCUGUACCC-3′, and the second spacer has a sequence that is at least about 75%, at least about 80%, at least about 85%, at least about 90%, at least about 95% identical to, or is identical to 5′-CAUUAUGGACAGAGUUACCG-3′,
wherein the first spacer has a sequence that is at least about 75%, at least about 80%, at least about 85%, at least about 90%, at least about 95% identical to, or is identical to 5′-AGGACACUCGAUUCUGUCA-3′, and the second spacer has a sequence that is at least about 75%, at least about 80%, at least about 85%, at least about 90%, at least about 95% identical to, or is identical to 5′-CACAACUCCCACAGUGGCCC-3′,
wherein the first spacer has a sequence that is at least about 75%, at least about 80%, at least about 85%, at least about 90%, at least about 95% identical to, or is identical to 5′-CUGUCACUGGACAUUGUGUC-3′, and the second spacer has a sequence that is at least about 75%, at least about 80%, at least about 85%, at least about 90%, at least about 95% identical to, or is identical to 5′-AAGUGUCCUUGCGACGUCCA-3′,
wherein the first spacer has a sequence that is at least about 75%, at least about 80%, at least about 85%, at least about 90%, at least about 95% identical to, or is identical to 5′-GGAGCAAAGCCCCACAGUCC-3′, and the second spacer has a sequence that is at least about 75%, at least about 80%, at least about 85%, at least about 90%, at least about 95% identical to, or is identical to 5′-GUUGGUGCUGAAAUUCAAAG-3′,
wherein the first spacer has a sequence that is at least about 75%, at least about 80%, at least about 85%, at least about 90%, at least about 95% identical to, or is identical to 5′-GGAGCAAAGCCCCGGGGUCC-3′, and the second spacer has a sequence that is at least about 75%, at least about 80%, at least about 85%, at least about 90%, at least about 95% identical to, or is identical to 5′-GUUGGUGCUGAAAUUCAAAG-3′,
wherein the first spacer has a sequence that is at least about 75%, at least about 80%, at least about 85%, at least about 90%, at least about 95% identical to, or is identical to 5′-CUGGAACUGGGACCACCGU-3′, and the second spacer has a sequence that is at least about 75%, at least about 80%, at least about 85%, at least about 90%, at least about 95% identical to, or is identical to 5′-ACAGAGCUUCCUUCUGAAGA-3′,
wherein the first spacer has a sequence that is at least about 75%, at least about 80%, at least about 85%, at least about 90%, at least about 95% identical to, or is identical to 5′-AUGCCAGUGUGGUGUCAUAG-3′, and the second spacer has a sequence that is at least about 75%, at least about 80%, at least about 85%, at least about 90%, at least about 95% identical to, or is identical to 5′-ACCACAGAAUACUACCCAAA-3′,
wherein the first spacer has a sequence that is at least about 75%, at least about 80%, at least about 85%, at least about 90%, at least about 95% identical to, or is identical to 5′-GGAGCCAGAAUAACAUUCGG-3′, and the second spacer has a sequence that is at least about 75%, at least about 80%, at least about 85%, at least about 90%, at least about 95% identical to, or is identical to 5′-CUAGAGGCUUUUUUUGAACA-3′;
wherein the first spacer has a sequence that is at least about 75%, at least about 80%, at least about 85%, at least about 90%, at least about 95% identical to, or is identical to 5′-CAGAUGCUGAGAUUAGUCCU-3′, and the second spacer has a sequence that is at least about 75%, at least about 80%, at least about 85%, at least about 90%, at least about 95% identical to, or is identical to 5′-UGGAUUCCUGCAGUAGUUCC-3′,
wherein the first spacer has a sequence that is at least about 75%, at least about 80%, at least about 85%, at least about 90%, at least about 95% identical to, or is identical to 5′-UGACACCACAUUGGCAUCGG-3′, and the second spacer has a sequence that is at least about 75%, at least about 80%, at least about 85%, at least about 90%, at least about 95% identical to, or is identical to 5′-ACAUGUUCUUCCUGUGAUAG-3′,
wherein the first spacer has a sequence that is at least about 75%, at least about 80%, at least about 85%, at least about 90%, at least about 95% identical to, or is identical to 5′-CAUAGAUGACCAAGAUUGAC-3′, and the second spacer has a sequence that is at least about 75%, at least about 80%, at least about 85%, at least about 90%, at least about 95% identical to, or is identical to 5′-UGAUACCACACUGGCAUCAG-3′,
wherein the first spacer has a sequence that is at least about 75%, at least about 80%, at least about 85%, at least about 90%, at least about 95% identical to, or is identical to 5′-CCAUCACUGGACAUUGCGUC-3′, and the second spacer has a sequence that is at least about 75%, at least about 80%, at least about 85%, at least about 90%, at least about 95% identical to, or is identical to 5′-AACUCUCCUCACAACUCCCA-3′,
wherein the first spacer has a sequence that is at least about 75%, at least about 80%, at least about 85%, at least about 90%, at least about 95% identical to, or is identical to 5′-CUGCAUCUGAGCAUCGUGUC-3′, and the second spacer has a sequence that is at least about 75%, at least about 80%, at least about 85%, at least about 90%, at least about 95% identical to, or is identical to 5′-CGUCCCUCCGAAUGUUAUUC-3′,
wherein the first spacer has a sequence that is at least about 75%, at least about 80%, at least about 85%, at least about 90%, at least about 95% identical to, or is identical to 5′-AAACAGCCGUGGACGUCGCA-3′, and the second spacer has a sequence that is at least about 75%, at least about 80%, at least about 85%, at least about 90%, at least about 95% identical to, or is identical to 5′-UGAACAAGGUAAGAAGUCUC-3′,
wherein the first spacer has a sequence that is at least about 75%, at least about 80%, at least about 85%, at least about 90%, at least about 95% identical to, or is identical to 5′-ACAGAGGCUCCUUCUGAACA-3′, and the second spacer has a sequence that is at least about 75%, at least about 80%, at least about 85%, at least about 90%, at least about 95% identical to, or is identical to 5′-GCUUGGAACCGGGGCCACUG-3′,
wherein the first spacer has a sequence that is at least about 75%, at least about 80%, at least about 85%, at least about 90%, at least about 95% identical to, or is identical to 5′-AUGCCAGUGUGGUGUCAUAG-3′, and the second spacer has a sequence that is at least about 75%, at least about 80%, at least about 85%, at least about 90%, at least about 95% identical to, or is identical to 5′-ACAACAGAAUAUUAUCCAAA-3′,
wherein the first spacer has a sequence that is at least about 75%, at least about 80%, at least about 85%, at least about 90%, at least about 95% identical to, or is identical to 5′-CUAUGACACCACAUUGGCAU-3′, and the second spacer has a sequence that is at least about 75%, at least about 80%, at least about 85%, at least about 90%, at least about 95% identical to, or is identical to 5′-ACAUGUUCUUCCUGUGAUAG-3′,
wherein the first spacer has a sequence that is at least about 75%, at least about 80%, at least about 85%, at least about 90%, at least about 95% identical to, or is identical to 5′-AAUAACAUUCGGAGGGACGA-3′, and the second spacer has a sequence that is at least about 75%, at least about 80%, at least about 85%, at least about 90%, at least about 95% identical to, or is identical to 5′-UAUUCUGGCUCCAAGCCUAG-3′,
wherein the first spacer has a sequence that is at least about 75%, at least about 80%, at least about 85%, at least about 90%, at least about 95% identical to, or is identical to 5′-GUAGCAGUCCUGUACCCCGG-3′, and the second spacer has a sequence that is at least about 75%, at least about 80%, at least about 85%, at least about 90%, at least about 95% identical to, or is identical to 5′-CAUUAUGGACAGAGUUACCG-3′,
wherein the first spacer has a sequence that is at least about 75%, at least about 80%, at least about 85%, at least about 90%, at least about 95% identical to, or is identical to 5′-AGUAGCAGUCCUGUACCCCG-3′, and the second spacer has a sequence that is at least about 75%, at least about 80%, at least about 85%, at least about 90%, at least about 95% identical to, or is identical to 5′-CAUUAUGGACAGAGUUACCG-3′,
wherein the first spacer has a sequence that is at least about 75%, at least about 80%, at least about 85%, at least about 90%, at least about 95% identical to, or is identical to 5′-UAGUAGCAGUCCUGUACCCC-3′, and the second spacer has a sequence that is at least about 75%, at least about 80%, at least about 85%, at least about 90%, at least about 95% identical to, or is identical to 5′-CAUUAUGGACAGAGUUACCG-3′, or
wherein the first spacer has a sequence that is at least about 75%, at least about 80%, at least about 85%, at least about 90%, at least about 95% identical to, or is identical to 5′-UGGACCACAUGGCUUUGCUC-3′, and the second spacer has a sequence that is at least about 75%, at least about 80%, at least about 85%, at least about 90%, at least about 95% identical to, or is identical to 5′-ACGUACUCCACCACUGUCAC-3′.

5. The pharmaceutical composition of claim 1,
wherein the first spacer has a sequence comprising nucleotides 6 to 20, 5 to 20, 4 to 20, 3 to 20, 2 to 20, or 1 to 20 of the following sequence 5′-CUGUCACCAGGCAUUGUGUC-3′, and the second spacer has a sequence comprising nucleotides 6 to 20, 5 to 20, 4 to 20, 3 to 20, 2 to 20, or 1 to 20 of the following sequence 5′-UGUCCUUGCAACUCUCACGG-3′,
wherein the first spacer has a sequence comprising nucleotides 6 to 20, 5 to 20, 4 to 20, 3 to 20, 2 to 20, or 1 to 20 of the following sequence 5′-GUAGUAGCAGUCCUGUACCC-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
wherein the first spacer has a sequence comprising nucleotides 6 to 20, 5 to 20, 4 to 20, 3 to 20, 2 to 20, or 1 to 20 of the following sequence 5′-AGGACACUCGAUUCUGUCA-3′, and the second spacer has a sequence comprising nucleotides 6 to 20, 5 to 20, 4 to 20, 3 to 20, 2 to 20, or 1 to 20 of the following sequence 5′-CACAACUCCCACAGUGGCCC-3′,
wherein the first spacer has a sequence comprising nucleotides 6 to 20, 5 to 20, 4 to 20, 3 to 20, 2 to 20, or 1 to 20 of the following sequence 5′-CUGUCACUGGACAUUGUGUC-3′, and the second spacer has a sequence comprising nucleotides 6 to 20, 5 to 20, 4 to 20, 3 to 20, 2 to 20, or 1 to 20 of the following sequence 5′-AAGUGUCCUUGCGACGUCCA-3′,
wherein the first spacer has a sequence comprising nucleotides 6 to 20, 5 to 20, 4 to 20, 3 to 20, 2 to 20, or 1 to 20 of the following sequence 5′-GGAGCAAAGCCCCACAGUCC-3′, and the second spacer has a sequence comprising nucleotides 6 to 20, 5 to 20, 4 to 20, 3 to 20, 2 to 20, or 1 to 20 of the following sequence 5′-GUUGGUGCUGAAAUUCAAAG-3′,
wherein the first spacer has a sequence comprising nucleotides 6 to 20, 5 to 20, 4 to 20, 3 to 20, 2 to 20, or 1 to 20 of the following sequence 5′-GGAGCAAAGCCCCGGGGUCC-3′, and the second spacer has a sequence comprising nucleotides 6 to 20, 5 to 20, 4 to 20, 3 to 20, 2 to 20, or 1 to 20 of the following sequence 5′-GUUGGUGCUGAAAUUCAAAG-3′,
wherein the first spacer has a sequence comprising nucleotides 6 to 20, 5 to 20, 4 to 20, 3 to 20, 2 to 20, or 1 to 20 of the following sequence 5′-CUGGAACUGGGACCACCGU-3′, and the second spacer has a sequence comprising nucleotides 6 to 20, 5 to 20, 4 to 20, 3 to 20, 2 to 20, or 1 to 20 of the following sequence 5′-ACAGAGCUUCCUUCUGAAGA-3′,
wherein the first spacer has a sequence comprising nucleotides 6 to 20, 5 to 20, 4 to 20, 3 to 20, 2 to 20, or 1 to 20 of the following sequence 5′-AUGCCAGUGUGGUGUCAUAG-3′, and the second spacer has a sequence comprising nucleotides 6 to 20, 5 to 20, 4 to 20, 3 to 20, 2 to 20, or 1 to 20 of the following sequence 5′-ACCACAGAAUACUACCCAAA-3′,
wherein the first has a spacer sequence comprising nucleotides 6 to 20, 5 to 20, 4 to 20, 3 to 20, 2 to 20, or 1 to 20 of the following sequence 5′-GGAGCCAGAAUAACAUUCGG-3′, and the second spacer has a sequence comprising nucleotides 6 to 20, 5 to 20, 4 to 20, 3 to 20, 2 to 20, or 1 to 20 of the following sequence 5′-CUAGAGGCUUUUU UUGAACA-3′;
wherein the first spacer has a sequence comprising nucleotides 6 to 20, 5 to 20, 4 to 20, 3 to 20, 2 to 20, or 1 to 20 of the following sequence 5′-CAGAUGCUGAGAUUAGUCCU-3′, and the second spacer has a sequence comprising nucleotides 6 to 20, 5 to 20, 4 to 20, 3 to 20, 2 to 20, or 1 to 20 of the following sequence 5′-UGGAUUCCUGCAGUAGUUCC-3′,
wherein the first spacer has a sequence comprising nucleotides 6 to 20, 5 to 20, 4 to 20, 3 to 20, 2 to 20, or 1 to 20 of the following sequence 5′-UGACACCACAUUGGCAUCGG-3′, and the second spacer has a sequence comprising nucleotides 6 to 20, 5 to 20, 4 to 20, 3 to 20, 2 to 20, or 1 to 20 of the following sequence 5′-ACAUGUUCUUCCUGUGAUAG-3′,
wherein the first spacer has a sequence comprising nucleotides 6 to 20, 5 to 20, 4 to 20, 3 to 20, 2 to 20, or 1 to 20 of the following sequence 5′-CAUAGAUGACCAAGAUUGAC-3′, and the second spacer has a sequence comprising nucleotides 6 to 20, 5 to 20, 4 to 20, 3 to 20, 2 to 20, or 1 to 20 of the following sequence 5′-UGAUACCACACUGGCAUCAG-3′,
wherein the first spacer has a sequence comprising nucleotides 6 to 20, 5 to 20, 4 to 20, 3 to 20, 2 to 20, or 1 to 20 of the following sequence 5′-CCAUCACUGGACAUUGCGUC-3′, and the second spacer has a sequence comprising nucleotides 6 to 20, 5 to 20, 4 to 20, 3 to 20, 2 to 20, or 1 to 20 of the following sequence 5′-AACUCUCCUCACAACUCCCA-3′,
wherein the first spacer has a sequence comprising nucleotides 6 to 20, 5 to 20, 4 to 20, 3 to 20, 2 to 20, or 1 to 20 of the following sequence 5′-CUGCAUCUGAGCAUCGUGUC-3′, and the second spacer has a sequence comprising nucleotides 6 to 20, 5 to 20, 4 to 20, 3 to 20, 2 to 20, or 1 to 20 of the following sequence 5′-CGUCCCUCCGAAUGUUAUUC-3′,
wherein the first spacer has a sequence comprising nucleotides 6 to 20, 5 to 20, 4 to 20, 3 to 20, 2 to 20, or 1 to 20 of the following sequence 5′-AAACAGCCGUGGACGUCGCA-3′, and the second spacer has a sequence comprising nucleotides 6 to 20, 5 to 20, 4 to 20, 3 to 20, 2 to 20, or 1 to 20 of the following sequence 5′-UGAACAAGGUAAGAAGUCUC-3′,
wherein the first spacer has a sequence comprising nucleotides 6 to 20, 5 to 20, 4 to 20, 3 to 20, 2 to 20, or 1 to 20 of the following sequence 5′-ACAGAGGCUCCUUCUGAACA-3′, and the second spacer has a sequence comprising nucleotides 6 to 20, 5 to 20, 4 to 20, 3 to 20, 2 to 20, or 1 to 20 of the following sequence 5′-GCUUGGAACCGGGGCCACUG-3′,
wherein the first spacer has a sequence comprising nucleotides 6 to 20, 5 to 20, 4 to 20, 3 to 20, 2 to 20, or 1 to 20 of the following sequence 5′-AUGCCAGUGUGGUGUCAUAG-3′, and the second spacer has a sequence comprising nucleotides 6 to 20, 5 to 20, 4 to 20, 3 to 20, 2 to 20, or 1 to 20 of the following sequence 5′-ACAACAGAAUAUUAUCCAAA-3′,
wherein the first spacer has a sequence comprising nucleotides 6 to 20, 5 to 20, 4 to 20, 3 to 20, 2 to 20, or 1 to 20 of the following sequence 5′-CUAUGACACCACAUUGGCAU-3′, and the second spacer has a sequence comprising nucleotides 6 to 20, 5 to 20, 4 to 20, 3 to 20, 2 to 20, or 1 to 20 of the following sequence 5′-ACAUGUUCUUCCUGUGAUAG-3′,
wherein the first spacer has a sequence comprising nucleotides 6 to 20, 5 to 20, 4 to 20, 3 to 20, 2 to 20, or 1 to 20 of the following sequence 5′-AAUAACAUUCGGAGGGACGA-3′, and the second spacer has a sequence comprising nucleotides 6 to 20, 5 to 20, 4 to 20, 3 to 20, 2 to 20, or 1 to 20 of the following sequence 5′-UAUUCUGGCUCCAAGCCUAG-3′,
wherein the first spacer has a sequence comprising nucleotides 6 to 20, 5 to 20, 4 to 20, 3 to 20, 2 to 20, or 1 to 20 of the following sequence 5′-GUAGCAGUCCUGUACCCCGG-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
wherein the first spacer has a sequence comprising nucleotides 6 to 20, 5 to 20, 4 to 20, 3 to 20, 2 to 20, or 1 to 20 of the following sequence 5′-AGUAGCAGUCCUGUACCCCG-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′,
wherein the first spacer has a sequence comprising nucleotides 6 to 20, 5 to 20, 4 to 20, 3 to 20, 2 to 20, or 1 to 20 of the following sequence 5′-UAGUAGCAGUCCUGUACCCC-3′, and the second spacer has a sequence comprising nucleotides 6 to 20, 5 to 20, 4 to 20, 3 to 20, 2 to 20, or 1 to 20 of the following sequence 5′-CAUUAUGGACAGAGUUACCG-3′, or
wherein the first spacer has a sequence comprising nucleotides 6 to 20, 5 to 20, 4 to 20, 3 to 20, 2 to 20, or 1 to 20 of the following sequence 5′-UGGACCACAUGGCUUUGCUC-3′, and the second spacer has a sequence comprising nucleotides 6 to 20, 5 to 20, 4 to 20, 3 to 20, 2 to 20, or 1 to 20 of the following sequence 5′-ACGUACUCCACCACUGUCAC-3′.

6. The pharmaceutical composition of claim 1, wherein the first spacer and/or the second spacer comprise a modified nucleotide.

7. The pharmaceutical composition of claim 6, wherein one or more nucleotide within five nucleotides from the 5′ end of the first spacer and/or the second spacer are modified nucleotides.

8. The pharmaceutical composition of claim 6, wherein each of the nucleotides within three nucleotides of the 5′ end of the first spacer and/or the second spacer are modified nucleotides.

9. The pharmaceutical composition of claim 6, wherein the modified nucleotide comprises a 2′-OMe modification and/or a phosphorothioate group.

10. The pharmaceutical composition of claim 1, wherein the one or more nickases comprise a CRSPR Cas nickase.

11. The pharmaceutical composition of claim 1, wherein at least one of the one or more Cas nickases, when in operative interaction with the first or second guide oligonucleotide, is engineered to nick the opposite strand of the LPA gene to which the operative guide oligonucleotide (gRNA) is hybridized.

12. The pharmaceutical composition of claim 11, wherein at least one of the one or more polynucleotides (mRNAs) encoding the one or more Cas nickases encodes a Streptococcus pyogenes Cas9 nickase bearing a H840A mutation.

13. The pharmaceutical composition of claim 1, wherein at least one of the one or more Cas nickases, when in operative interaction with the first or second guide oligonucleotide (gRNA), is engineered to nick the same strand of the LPA gene to which the operative guide is hybridized.

14. The pharmaceutical composition of claim 13, wherein at least one of the one or more polynucleotides (mRNAs) encoding the one or more Cas nickases encodes a Streptococcus pyogenes Cas9 nickase bearing a D10A mutation.

15. The pharmaceutical composition of claim 1, wherein at least one of the one or more polynucleotides (mRNAs) comprises:
(a) a 5′ untranslated region (UTR);
(b) a 3′ UTR region;
(c) a poly(A) tail proximate to the 3′ UTR as compared to the 5′ UTR, said poly(A) tail comprising a chain of 80-150 nucleotides length that comprise adenine nucleotides; and
(d) a gene editor coding region encoding an impaired CRISPR Cas9 endonuclease domain and a polymerase domain, said gene editor coding region extending between the 5′ UTR and the 3′ UTR.

16. The pharmaceutical composition of claim 1, wherein at least one of the one or more polynucleotides is selected from any of the mRNA sequences listed in Table 1 or an mRNA having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the mRNA sequences listed in Table 1.

17. The pharmaceutical composition of claim 1, wherein at least one of the one or more polynucleotides comprises a coding sequence of any of the mRNA sequences listed in Table 1 or comprises a coding sequence having at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any of the coding sequences of the nickase mRNA sequences listed in Table 1.

18. The pharmaceutical composition of claim 1, wherein the LNPs comprise:
(a) one or more ionizable lipids,
(b) cholesterol,
(c) one or more PEG-lipids,
(d) a phospholipid; and
(e) optionally including a targeting moiety, such as a GalNAc lipid.

19. The pharmaceutical composition of claim 18, wherein the LNPs are formulated to comprise:
(a) 40 to 60 molar percent of the one or more ionizable lipids, 28 to 48 molar percent of the cholesterol, 5 to 13 molar percent of the phospholipid, and 2 to 5 molar percent of the PEG-lipid,
(b) 50+/−10% molar percent of the one or more ionizable lipids, 38+/−10% molar percent of the cholesterol, 9+/−10% molar percent of the phospholipid, and 3+/−10% molar percent of the PEG-lipid,
(c) 40 to 60 molar percent of the one or more ionizable lipids, 27.95 to 47.95 molar percent of the cholesterol, 5 to 13 molar percent of the phospholipid, 2 to 5 molar percent of the PEG-lipid, and 0.02 to 0.09 molar percent of the GalNAc-lipid; or
(d) 50+/−10% molar percent of the one or more ionizable lipids, 37.95+/−10% molar percent of the cholesterol, 9+/−10% molar percent of the phospholipid, 3+/−10% molar percent of the PEG-lipid, and 0.05+/−10% molar percent of the GalNAc-lipid.

20. The pharmaceutical composition of claim 1, wherein the sequences of the first and second scaffold region are the same.

21. The pharmaceutical composition of claim 1, wherein the scaffold region sequences of the first and second guide oligonucleotides (gRNAs) are each independently selected from one of the following sequences or a sequence having at least about 75%, at least about 80%, at least about 85%, at least about 90%, or at least about 95% similarity or identity to one of the following sequences:
5′-GUUUUAGAGCUAGAAAUAGCAAGUUAAAAUAAGGCUAGUCCGUUAUC
AACUUGAAAAAGUGGCACCGAGUCGGUGC-3′,
and
5′-GUUUGAGAGCUAUGCUGGAAACAGCAUAGCAAGUUCAAAUAAGGCUA
GUCCGUUAUCAACUUGAAAAAGUGGCACCGAGUCGGUGC-3′.

22. The pharmaceutical composition of claim 1, wherein the first scaffold and/or the second scaffold comprise a modified nucleotide.

23. The pharmaceutical composition of claim 1, wherein the scaffold comprises one or more of the modified nucleotides in the following nucleotide sequence: 5′-mGUUUUAGmAmGmCmUmAGmAmAmAmUmAmGmCmAmAGUUmAAmAAmU AmAmGmGmCmUmAGUmCmCGUUAmUmCAAmCmUmUGmAmAmAmAmAmG mUmGGmCmAmCmCmGmAmGmUmCmGmGmUmGmC 3′, where m of mN is 2′-O-methyl ribose.

24. The pharmaceutical composition of claim 1, wherein the scaffold comprises the following sequence or a sequence having at least about 75%, at least about 80%, at least about 85%, at least about 90%, or at least about 95% identity to following sequence:
5′-mGUUUUAGmAmGmCmUmAGmAmAmAmUmAmGmCmAmAGUUmAAmAAmU AmAmGmGmCmUmAGUmCmCGUUAmUmCAAmCmUmUGmAmAmAmAmAmG mUmGGmCmAmCmCmGmAmGmUmCmGmGmUmGmC 3′, where m of mN is 2′-O-methyl ribose.

25. The pharmaceutical composition of claim 1, wherein the first guide oligonucleotide and/or the second guide oligonucleotide comprise an RNA motif at the 3′ end of the guide oligonucleotide.

26. The pharmaceutical composition of claim 25, wherein the RNA motif comprises, consists essentially of, or consists of 5′-UUU-3′.

27. The pharmaceutical composition of claim 25, wherein the RNA motif comprises, consists essentially of, or consists of 5′-*mU*mU*mU-3′, where “mU*” indicates a phosphorothioated 2′-O-methyl uracil base, and “mU” indicates a 2′-O-methyl uracil base.

28. The pharmaceutical composition of claim 1, wherein the weight ratio of the total weight of the first and second guide oligonucleotides to the weight of the mRNA is 1:1+/−25%.

29. The pharmaceutical composition of claim 1, wherein the weight ratio of the first guide oligonucleotide to the second guide oligonucleotide is 1:1+/−25%.

30. The pharmaceutical composition of claim 2, wherein the first location and the second location are spaced apart by less than 200 nucleotides.

31. The pharmaceutical composition of claim 30, wherein the first location and the second location are spaced apart by a distance of 20 to 50 nucleotides.

32. The pharmaceutical composition according to claim 1, wherein administration of the composition to hepatocytes results in 40% or greater editing efficiency.

33. The pharmaceutical composition according to claim 32, wherein the hepatocytes are primary hepatocytes.

34. The pharmaceutical composition according to claim 32, wherein the hepatocytes are human hepatocytes.

35. A method for inactivating the LPA gene in vivo in a mammalian subject to treat and prevent cardiovascular disease comprising the step of: administering to the subject a pharmaceutical composition of claim 1.

36. A method for reducing blood Lp(a) concentration in a mammalian subject to treat and prevent cardiovascular disease comprising the step of: administering to the subject a pharmaceutical composition of claim 1.

37. A method for treating and/or preventing cardiovascular disease associated with the LPA gene in a mammalian subject comprising the step of:
administering to the subject a pharmaceutical composition of claim 1.

38. A method for in vivo editing of an LPA gene in a mammalian subject comprising the step of: administering to the subject a pharmaceutical composition of claim 1.

39. A method for in vivo editing of an LPA gene in a mammalian subject comprising:
administering to the subject a pharmaceutical composition comprising:
(i) one or more polynucleotides (mRNAs) encoding one or more CRISPR Cas nickases,
(ii) a first guide oligonucleotide (gRNA) comprising a first spacer sequence and a scaffold region; and
(iii) a second guide oligonucleotide (gRNA) comprising a second spacer sequence and a scaffold region, and
(iv) a delivery system that is engineered to deliver the one or more mRNAs, the first gRNA, and/or the second gRNA individually or collectively to the liver,
wherein the first gRNA and at least one of the one or more Cas nickases are engineered to cause the at least one of the one or more Cas nickases to nick one of the first or second strands of the LPA gene at a first location of chromosome 6 from position 160,664,275 to 160,531,482, and
wherein the second gRNA and at least one of the one or more Cas nickases are engineered to cause the at least one of the one or more Cas nickases to nick the other of the first or second strands of the LPA gene at a second location of chromosome 6 from position 160,664,275 to160,531,482.

40. A gene editing system for editing the LPA gene produced by expressing in a cell one or more exogenous polynucleotides (mRNA) encoding one or more CRISPR Cas nickases and introducing first and second gRNAs into the cell,
wherein the first guide oligonucleotide (gRNA) comprises (i) a first spacer sequence that is complementary to a first strand of the LPA gene at a first target sequence and (ii) a first scaffold region that serves as a binding scaffold for at least one of the one or more Cas nickases,
wherein the second guide oligonucleotide (gRNA) comprises (i) a second spacer sequence that is complementary to a second strand of the LPA gene at a second target sequence and (ii) a second scaffold region that serves as a binding scaffold for at least one of the one or more Cas nickases,
wherein the first gRNA and the at least one of the one or more Cas nickases are engineered to cause the at least one of the one or more Cas nickases to nick one of the first or second strands of the LPA gene at a first location of chromosome 6 from position 160,664,275 to 160,531,482, and
wherein the second gRNA and the at least one of the one or more Cas nickase are engineered to cause the at least one of the one or more Cas nickases to nick the other of the first or second strands of the LPA gene at a second location of chromosome 6 from position 160,664,275 to 160,531,482.

41. A gene editing system comprising:
a means for expressing one or more CRISPR Cas nickases in a cell; and
a means for directing the one or more Cas nickases to first and second locations in the LPA gene and to cause the one or more Cas nickases to introduce a nick in a first strand of the LPA gene and to introduce a nick in a second strand of the LPA gene.

42. A pharmaceutical composition comprising the gene editing system of claim 41 and a delivery system.

43. The pharmaceutical composition of claim 42, wherein the delivery system comprises means for delivering to the cell the means for expressing one or more CRISPR Cas nickases and the means for directing the one or more Cas nickases to first and second locations in the LPA gene.

44. A gene editing system for editing the LPA gene comprising:
a Cas nickase or a nucleic acid encoding the Cas nickase;
a first guide oligonucleotide comprising (i) a first spacer sequence that is complementary to a first strand of the LPA gene at a first target sequence and (ii) a first scaffold region that serves as a binding scaffold for the Cas nickase;
a second guide oligonucleotide comprising (i) a second spacer sequence that is complementary to a second strand of the LPA gene at a second target sequence and (ii) a second scaffold region that serves as a binding scaffold for the Cas nickase,
wherein the first guide oligonucleotide and the Cas nickase are engineered to cause the Cas nickase to nick one of the first or second strands of the LPA gene at a first location,
wherein the second guide oligonucleotide and the Cas nickase are engineered to cause the Cas nickase to nick the other of the first or second strands of the LPA gene at a second location, and
wherein the first location and the second location are spaced apart by a distance of 1 to 200 nucleotides.

45. The gene editing system of claim 44, wherein the first location and the second location are spaced apart by a distance of 20 to 50 nucleotides.
